(19) United States
(12) Patent Application Publication
(10) Pub. No.: US 2024/0251810 A1
(43) Pub. Date: Aug. 1, 2024

(54) INCREASED YIELD OF MILK PROTEIN PER ACRE

(71) Applicant: Nobell Foods, Inc., South San Francisco, CA (US)

(72) Inventors: Magi EL-RICHANI, San Francisco, CA (US); Viviane LANQUAR, San Carlos, CA (US); Joseph KLEMASZEWSKI, Eden Prairie, MN (US); Yaxin WANG, South San Francisco, CA (US); Sunchung PARK, South San Francisco, CA (US); Alexander CAIN, South San Francisco, CA (US); Leyla HATHWAIK, South San Francisco, CA (US); Jacob William HOYLE, South San Francisco, CA (US)

(21) Appl. No.: 18/598,187

(22) Filed: Mar. 7, 2024

Related U.S. Application Data
(63) Continuation of application No. PCT/US2022/077440, filed on Sep. 30, 2022.
(60) Provisional application No. 63/325,564, filed on Mar. 30, 2022.
(60) Provisional application No. 63/250,600, filed on Sep. 30, 2021.

(51) Int. Cl. A23C 11/06; A23C 20/00; C07K 14/415; C07K 14/47

(52) U.S. Cl. CPC A23C 11/06; A23C 20/005; C07K 14/415; C07K 14/4732; A23C 2240/15

(57) ABSTRACT

The present disclosure provides a solution to meet the world's growing milk and milk protein needs, while also balancing resource utilization, providing minimal impacts upon environmental systems, and moving away from an inhumane disregard for animal welfare.

### CROSS REFERENCE TO RELATED APPLICATIONS

The present application is a continuation of International Application No. PCT/US2022/077440, filed Sep. 30, 2022, which claims priority to U.S. Provisional Patent Application No. 63/325,564, filed Mar. 30, 2022, and U.S. Provisional Patent Application No. 63/250,600, filed Sep. 30, 2021, each of which is incorporated by reference herein in their entirety for all purposes.

### REFERENCE TO AN ELECTRONIC SEQUENCE LISTING

The contents of the electronic sequence listing (ALRO_011_04US_SeqList_ST26.xml; Size: 1,602,807 bytes; and Date of Creation: Mar. 6, 2024) is herein incorporated by reference in its entirety.

### FIELD OF THE DISCLOSURE

The present disclosure generally relates to genetically modified plants with increased expression of mammalian milk proteins and agronomic practices for producing same. The disclosure further relates to edible compositions comprising novel ratios of mammalian proteins to plant-based proteins. Methods of modulating fatty acid profiles, and modified products produced therefrom are also disclosed.

### BACKGROUND OF THE DISCLOSURE

Globally, more than 7.5 billion people around the world consume milk and other dairy products. It is estimated that cow milk accounts for 83% of global milk production and the demand for milk and dairy products is expected to keep increasing, in a commensurate manner with the growth in human population, which is expected to exceed 9 billion people by 2050.

Relying on the inhumane and environmentally detrimental practice of animal agriculture to meet the growing demand for milk and dairy products is not sustainable. According to the Food & Agriculture Organization of the United Nations, animal agriculture is responsible for 18% of all greenhouse gases, which is more than the entire transportation sector combined. Dairy cows alone account for 3% of this total.

Accordingly, in order to meet the world's growing milk and milk protein needs—while also balancing resource utilization, providing minimal impacts upon environmental systems, and moving away from an inhumane disregard for animal welfare—a better approach to producing milk proteins is urgently needed.

### BRIEF SUMMARY OF THE DISCLOSURE

The disclosure provides a solution that allows for safe, sustainable, and humane production of milk proteins. The disclosure provided herein teaches plants, compositions, and methods, which enable increased production of recombinant proteins (e.g., milk proteins).

In aspects, the disclosure provides a method for increasing yield of recombinant milk protein production per acre in a plant, comprising: providing to a locus a plurality of transgenic plant seed, wherein the transgenic plant seed comprise a recombinant DNA construct encoding a fusion protein comprising at least one milk protein, and wherein the plurality of transgenic plant seed produce in the aggregate at least 2 pounds of recombinant milk protein per acre. In aspects, the plurality of transgenic plant seed produce in the aggregate at least 5, 10, 20, 30, 40, 50, 60, 70, 80, 90, 100, 150, 200, 250, 300, 350, or 400 pounds of recombinant milk protein per acre

In aspects, the disclosure provides a method for increasing yield of recombinant milk protein production per acre in a plant, comprising: providing to a locus a plurality of genetically modified plant seed, wherein the plurality of genetically modified plant seed produce in the aggregate at least 2 pounds of recombinant milk protein per acre. In aspects, the plurality of genetically modified plant seed produce in the aggregate at least 5, 10, 20, 30, 40, 50, 60, 70, 80, 90, 100, 150, 200, 250, 300, 350, or 400 pounds of recombinant milk protein per acre

In aspects, the plant seed comprises at least one of the following genetic modifications: a recombinant DNA construct encoding a fusion protein comprising at least one milk protein; a recombinant DNA construct encoding a protein capable of forming a protein body; a recombinant DNA construct encoding a prolamin; a first recombinant DNA construct encoding a milk protein and a second recombinant DNA construct encoding a prolamin; a recombinant DNA construct encoding a milk protein that has been modified to have an amino acid sequence different from the native animal expressed milk protein; a recombinant DNA construct encoding a milk protein that has been modified to promote addition of a post-translational modification; a recombinant DNA construct encoding a milk protein that has been modified to prevent addition of a post-translational modification; a recombinant DNA construct encoding an enzyme that alters post-translational modification of protein; a recombinant DNA construct encoding an enzyme capable of modifying a protein; a recombinant DNA construct encoding a kinase; and/or a genetic modification that modulates the expression of a plant protease. The plant may have any one, or all, of the aforementioned modifications. The plant may be a monocot or dicot. The plant may be a soybean plant.

Provided herein are recombinant fusion proteins comprising (i) a first milk protein, and (ii) a second milk protein. At least one of the first milk protein and the second milk protein may be, for example, α-S1 casein, α-S2 casein, β-casein, κ-casein, para-κ-casein, β-lactoglobulin, α-lactalbumin, lysozyme, lactoferrin, lactoperoxidase, serum albumin, or an immunoglobulin. In some embodiments, at least one of the first milk protein and the second milk protein is β-lactoglobulin. In some embodiments, at least one of the first milk protein and the second milk protein is α-S1 casein, α-S2 casein, β-casein, κ-casein, or para-κ-casein. In some embodiments, i) the first milk protein is α-S1 casein, α-S2 casein, β-casein, κ-casein, or para-κ-casein; and ii) the second milk protein is α-S1 casein, α-S2 casein, β-casein, κ-casein, or para-κ-casein. In some embodiments, at least one of the first milk protein and the second milk protein is κ-casein and comprises the sequence of SEQ ID NO: 4, or a sequence at least 90% identical thereto. In some embodiments, at least one of the first milk protein and the second milk protein is para-κ-casein and comprises the sequence of SEQ ID NO: 2, or a sequence at least 90% identical thereto. In some embodiments, at least one of the first milk protein and the second milk protein is β-casein and comprises the sequence of SEQ ID NO: 6, or a sequence at least 90% identical thereto. In some embodiments, at least one of the first milk protein and the second milk protein is α-S1 casein and comprises the sequence SEQ ID NO: 8, or a sequence at least 90% identical thereto. In some embodiments, at least one of the first milk protein and the second milk protein is α-S2 casein and comprises the sequence SEQ ID NO: 84, or a sequence at least 90% identical thereto. In some embodiments, the first milk protein and the second milk protein are different proteins. In some embodiments, the first milk protein and the second milk protein are the same proteins. In some embodiments, the fusion protein is plant-expressed. In some embodiments, the fusion protein is expressed in soybean plant. In some embodiments, the fusion protein comprises a protease cleavage site. In some embodiments, the protease cleavage site is a chymosin cleavage site.

Also provided herein are nucleic acids encoding one or more of the recombinant fusion proteins of the disclosure, and expression vectors comprising the same. In some embodiments, the nucleic acids are codon-optimized for expression in a plant, such as a soybean.

Additionally, provided herein are host cells comprising a nucleic acid or an expression vector of the disclosure, i.e., a nucleic acid or expression vector encoding a fusion protein. The host cells may be, for example, plant cells, bacterial cells, fungal cells, or mammalian cells. In some embodiments, the host cells are soybean cells.

Also provided herein are plants stably transformed with a nucleic acid or an expression vector of the disclosure. In some embodiments, the fusion protein is expressed in the plant in an amount of 1% or higher per total protein weight of soluble protein extractable from the plant.

Also provided herein are methods for making a fusion protein, the methods comprising: (a) transforming a host cell with a nucleic acid or an expression vector described herein; and (b) growing the transformed host cell under conditions wherein the fusion protein is expressed. In some embodiments, the method comprises co-expressing in the host cell a protein capable of forming a protein body, such as a prolamin selected from a gliadin, a hordein, a secalin, a zein, a kafirin, or an avenin. In some embodiments, the method comprises expressing a kinase in the host cell. In some embodiments, expression of one or more proteases is knocked down or knocked out in the cell.

Also provided herein are transgenic plants comprising a recombinant fusion protein, or a nucleic acid or expression vector comprising the same. In some embodiments, the transgenic plant is a soybean plant. In some embodiments, the fusion protein is expressed in the plant in an amount of 1% or higher per total protein weight of soluble protein extractable from the plant.

Also provided herein are methods for stably expressing a recombinant fusion protein in a plant, the methods comprising: (i) transforming a plant with a plant transformation vector comprising an expression cassette comprising a nucleic acid molecule encoding the fusion protein; and (ii) growing the transformed plant under conditions wherein the recombinant fusion protein is expressed. In some embodiments, the fusion protein is expressed in an amount of 1% or higher per total protein weight of soluble protein extractable from the plant.

Also provided herein are seed processing compositions comprising a fusion protein of the disclosure.

Also provided herein are food compositions comprising a fusion protein of the disclosure. In some embodiments, the food composition is selected from the group consisting of cheese and processed cheese products, yogurt and fermented dairy products, directly acidified counterparts of fermented dairy products, cottage cheese dressing, frozen dairy products, frozen desserts, desserts, baked goods, toppings, icings, fillings, low-fat spreads, dairy-based dry mixes, soups, sauces, salad dressing, geriatric nutrition, creams and creamers, analog dairy products, follow-up formula, baby formula, infant formula, milk, dairy beverages, acid dairy drinks, smoothies, milk tea, butter, margarine, butter alternatives, growing up milks, low-lactose products and beverages, medical and clinical nutrition products, protein/nutrition bar applications, sports beverages, confections, meat products, analog meat products, meal replacement beverages, weight management food and beverages, cultured buttermilk, sour cream, yogurt, skyr, leben, lassi, kefir, powder containing a milk protein, and low-lactose products. In some embodiments, the food composition comprises a total amount of casein protein; wherein about 32% to 100% by weight of the total amount of casein protein in the food composition is beta-casein. In some embodiments, the food composition is a cheese composition. In some embodiments, the cheese composition has the ability to stretch to at least 3 cm in length without breaking, as determined by heating a 100-gram mass of the composition at a temperature of 225° C. for 4 minutes and cooling to about 90° C. and pulling with a fork placed beneath the mass.

Also provided herein is method of making a food composition, comprising combining a fusion protein disclosed herein into a food composition.

Also provided herein is an alternative dairy food composition comprising i) a recombinant fusion protein described herein; and ii) at least one lipid. In some embodiments, the recombinant fusion protein confers on the alternative dairy food composition one or more characteristics of a dairy food product selected from the group consisting of: taste, aroma, appearance, handling, mouthfeel, density, structure, texture, elasticity, springiness, coagulation, binding, leavening, aeration, foaming, creaminess, and emulsification. In some embodiments, the alternative dairy food composition does not comprise any other milk proteins. In some embodiments, the alternative dairy food composition comprises calcium at a concentration of about 0.01 to about 2% by weight. In some embodiments, the alternative dairy food composition comprises a total amount of casein protein; wherein about 32% to 100% by weight of the total amount of casein protein in the food composition is beta-casein. In some embodiments, the alternative diary food composition has a pH of about 5.2 to about 5.9. In some embodiments, the alternative dairy food composition is selected from the group consisting of cheese and processed cheese products, yogurt and fermented dairy products, directly acidified counterparts of fermented dairy products, cottage cheese dressing, frozen dairy products, frozen desserts, desserts, baked goods, toppings, icings, fillings, low-fat spreads, dairy-based dry mixes, soups, sauces, salad dressing, geriatric nutrition, creams and creamers, analog dairy products, follow-up formula, baby formula, infant formula, milk, dairy beverages, acid dairy drinks, smoothies, milk tea, butter, margarine, butter alternatives, growing up milks, low-lactose products and beverages, medical and clinical nutrition products, protein/nutrition bar applications, sports beverages, confections, meat products, analog meat products, meal replacement beverages, weight management food and beverages, cultured buttermilk, sour cream, yogurt, skyr, leben, lassi, kefir, powder containing a milk protein, and low-lactose products. In some embodiments, the alternative diary food composition is a cheese composition.

Also provided herein are solid phase, protein-stabilized emulsions comprising a fusion protein described herein, wherein the emulsions have the ability to stretch to at least 3 cm in length without breaking, as determined by heating a 100-gram mass of the emulsion to a temperature of about 225° C. for 4 minutes and cooling to about 90° C. and pulling with a fork placed beneath the mass.

Also provided herein are colloidal suspensions comprising a fusion protein described herein, wherein the colloidal suspension has at least one, at least two, or at least three characteristics that are substantially similar to bovine milk selected from taste, appearance, mouthfeel, structure, texture, density, elasticity, springiness, coagulation, binding, leavening, aeration, foaming, creaminess, and emulsification.

These and other embodiments are described in detail below.

### BRIEF DESCRIPTION OF THE DRAWINGS

The accompanying figures, which are incorporated herein and form a part of the specification, illustrate some, but not the only or exclusive, example embodiments and/or features. It is intended that the embodiments and figures disclosed herein are to be considered illustrative rather than limiting.

FIG. 1A-FIG. 1P show expression cassettes having different combinations of fusions between sequences encoding structured and intrinsically unstructured proteins (not to scale). Coding regions and regulatory sequences are indicated as blocks (not to scale). As used in the figures, “L” refers to linker; “Sig” refers to a signal sequence that directs foreign proteins to protein storage vacuoles, “5′ UTR” refers to the 5′ untranslated region, and “KDEL” refers to an endoplasmic reticulum retention signal.

FIG. 2A-FIG. 2P show expression cassettes having different combinations of fusions between sequences encoding a first protein and a second protein (not to scale), wherein the first and/or second protein is a milk protein (not shown). Coding regions and regulatory sequences are indicated as blocks (not to scale). As used in the figures, “L” refers to linker; “Sig” refers to a signal sequence that directs foreign proteins to protein storage vacuoles, “5′ UTR” refers to the 5′ untranslated region, and “KDEL” refers to an endoplasmic reticulum retention signal.

FIG. 3 shows the modified pAR15-00 binary vector containing a selectable marker cassette conferring herbicide resistance. Coding regions and regulatory sequences are indicated as blocks (not to scale).

FIG. 4 shows an example expression cassette comprising a OKC1-T:OLG1 fusion (Optimized Kappa Casein version 1:beta-lactoglobulin version 1, SEQ ID NOs: 71-72), expression of which is driven by PvPhas promoter fused with arc5′UTR:sig10, followed by the ER retention signal (KDEL) and the 3′UTR of the arc5-1 gene, “arc-terminator”. “arc5′UTR” refers to the 5′ untranslated region of the arc5-1 gene. “Sig10” refers to the lectin 1 gene signal peptide. “RB” refers to ribosomal binding site. Coding regions and regulatory sequences are indicated as blocks (not to scale).

FIG. 5 shows an example expression cassette comprising an OBC-T2:FM: OLG1 fusion (Optimized Beta Casein Truncated version 2: Chymosin cleavage site: beta-lactoglobulin version 1, SEQ ID NOs: 73-74), expression of which driven by PvPhas promoter fused with arc5′UTR: sig10, followed by the 3′UTR of the arc5-1 gene, “arc-terminator”. “arc5′UTR” refers to the 5′ untranslated region of the arc5-1 gene. “Sig10” refers to the lectin 1 gene signal peptide. “RB” refers to ribosomal binding site. Coding regions and regulatory sequences are indicated as blocks (not to scale). The Beta Casein is “truncated” in that the bovine secretion signal is removed and replaced with a plant targeting signal.

FIG. 6 shows an example expression cassette comprising a OaS1-T:FM: OLG1 fusion (Optimized Alpha S1 Casein Truncated version 1: Chymosin cleavage site: beta-lactoglobulin version 1, SEQ ID NOs: 75-76), expression of which is driven by PvPhas promoter fused with arc5′UTR: sig10, followed by the 3′UTR of the arc5-1 gene, “arc-terminator”. “arc5′UTR” refers to the 5′ untranslated region of the arc5-1 gene. “Sig10” refers to the lectin 1 gene signal peptide. “RB” refers to ribosomal binding site. Coding regions and regulatory sequences are indicated as blocks (not to scale). The Alpha S1 Casein is “truncated” in that the bovine secretion signal is removed and replaced with a plant targeting signal.

FIG. 7 shows an example expression cassette comprising a para-OKC1-T:FM: OLG1: KDEL fusion (Optimized paraKappa Casein version 1: Chymosin cleavage site: beta-lactoglobulin version 1, SEQ ID NOs: 77-78), expression of which is driven by PvPhas promoter fused with arc5′UTR: sig 10, followed by the ER retention signal (KDEL) and the 3′UTR of the arc5-1 gene, “arc-terminator”. “arc5′UTR” refers to the 5′ untranslated region of the arc5-1 gene. “Sig10” refers to the lectin 1 gene signal peptide. “RB” refers to ribosomal binding site. Coding regions and regulatory sequences are indicated as blocks (not to scale).

FIG. 8 shows an example expression cassette comprising a para-OKC1-T:FM: OLG1 fusion (Optimized paraKappa Casein version 1: Chymosin cleavage site: beta-lactoglobulin version 1, SEQ ID NOs: 79-80), expression of which is driven by PvPhas promoter fused with arc5′UTR: sig 10, followed by the 3′UTR of the arc5-1 gene, “arc-terminator.” “arc5′UTR” refers to the 5′ untranslated region of the arc5-1 gene. “Sig10” refers to the lectin 1 gene signal peptide. “RB” refers to ribosomal binding site. Coding regions and regulatory sequences are indicated as blocks (not to scale).

FIG. 9 shows an example expression cassette comprising a OKC1-T: OLG1 fusion (Optimized Kappa Casein version 1: beta-lactoglobulin version 1, SEQ ID NOs: 81-82), expression of which is driven by the promoter and signal peptide of glycinin 1 (GmSeed2: sig2) followed by the ER retention signal (KDEL) and the nopaline synthase gene termination sequence (nos term). Coding regions and regulatory sequences are indicated as blocks (not to scale).

FIG. 10A-FIG. 10D show protein detection by western blotting. FIG. 10A shows detection of the fusion protein using a primary antibody raised against κ-casein (kCN). The kCN commercial protein is detected at an apparent MW of ˜26 kDa (theoretical: 19 kDa—arrow). The fusion protein is detected at an apparent MW of ˜40 kDa (theoretical: 38 kDa—arrowhead). FIG. 10B shows detection of the fusion protein using a primary antibody raised against β-lactoglobulin (LG). The LG commercial protein is detected at an apparent MW of −18 kDa (theoretical: 18 kDa—arrow). The fusion protein is detected at an apparent MW of ˜40 kDa (theoretical: 38 kDa—arrowhead). FIG. 10C and FIG. 10D show protein gels as control for equal lane loading (image is taken at the end of the SDS run).

FIG. 11A-FIG. 11E provide a series of illustrations showing potential mechanisms by which casein proteins may be degraded in plant cells, and how fusion of a casein protein with a second protein (i.e., a fusion partner) may lead to accumulation thereof. KCN stands for kappa-casein, BC stands for beta casein, aS1 stands for alphα-S1 casein, aS2 stands for alpha-S2 casein, PTM stands for post-translational modification.

FIG. 12A and FIG. 12B show two illustrative fusion proteins. In FIG. 12A, a κ-casein protein is fused to a β-lactoglobulin protein. The κ-casein comprises a natural chymosin cleavage site (arrow 1). Cleavage of the fusion protein with rennet (or chymosin) yields two fragments: a para-kappa casein fragment, and a fragment comprising a κ-casein macropeptide fused to β-lactoglobulin. In some embodiments, a second protease cleavage site may be added at the C-terminus of the k-casein protein (i.e., at arrow 2), in order to further allow separation of the κ-casein macropeptide and the β-lactoglobulin. The second protease cleavage site may be a rennet cleavage site (e.g., a chymosin cleavage site), or it may be a cleavage site for a different protease. In FIG. 12B, a para-κ-casein protein is fused directly to β-lactoglobulin. A protease cleavage site (e.g., a chymosin cleavage site) is added between the para-κ-casein and the β-lactoglobulin to allow for separation thereof. By fusing the para-κ-casein directly to the β-lactoglobulin, no κ-casein macropeptide is produced upon cleavage of the fusion by chymosin (or other protease).

FIG. 13 is a flow-chart showing an illustrative process for producing a food composition comprising an unstructured milk protein, as described herein. Initially, an expression construct for expression of a fusion protein in a plant cell is designed. The construct is transformed into a plant, and the plant is regenerated. Seeds are collected from the plant, and processed (e.g., by seed hulling and grinding) to produce a seed processing composition. Protein is extracted, and optionally enriched and/or concentrated (i.e., to produce a protein concentrate composition). The extracted fusion protein may optionally be cleaved or used directly to produce a food composition.

FIG. 14A and FIG. 14B are images of a western blot used to detect kappa-casein protein (kCN) in samples comprising soybean total protein extracts (WT) and soybean total protein extracts spiked with 100 ng of KCN in the presence (WT+kCN+Halt) or absence (WT+kCN) of protease inhibitors. 5 μg of total protein was loaded in each lane. FIG. 14A shows protein detected using a primary antibody raised against KCN. FIG. 14B shows total protein, as a loading control (Stain-Free detection by Bio-Rad®).

FIG. 15A and FIG. 15B are images that show protein detection by western blotting. FIG. 15A shows detection of a fusion protein comprising β-casein and β-lactoglobulin using a primary antibody raised against β-casein (B-CN). Commercial protein was detected at an apparent MW of ˜30 kDa (arrowhead; theoretical: 23.5 kDa). The fusion protein was detected at an apparent MW of ˜40 kDa (arrow; theoretical: 42 kDa). FIG. 15B shows a protein gel as a control for equal lane loading, visualized using stain-free detection by Bio Rad® (image is taken at the end of the SDS run). 5 μg of total protein extracts were loaded per lane.

FIG. 16A-FIG. 16C shows expression of proteins according to the present disclosure. FIG. 16A shows molecular weight of various proteins, and levels of kappa-casein expression observed in transformed soybeans when those proteins are fused to the kappa-casein. FIG. 16B shows hydrophobicity of various proteins, and levels of kappa-casein expression observed in transformed soybeans when those proteins are fused to the kappa-casein. FIG. 16C shows flexibility of various proteins (i.e., number of disulfide bonds), and levels of kappa-casein expression observed in transformed soybeans when those proteins are fused to the kappa-casein. Expression levels shown in FIG. 16A-FIG. 16C are relative to kappa casein expressed alone (i.e., not as a fusion, KCN only). The values for % KCN only are presented as a log₁₀ scale. Values above 100% indicate that kappa-casein was stabilized by the fusion.

FIG. 17 is a schematic showing an illustrative process for producing a food composition. The food composition produced according to this method may comprise one or more of: (i) one or more constituent proteins derived from a fusion protein, (ii) the fusion protein itself, or (ii) other protein extracted from the seed that was used to produce the fusion protein.

FIG. 18 is a schematic that shows how knocking-down or knocking-out the expression and/or activity of one or more proteases in a plant seed may prevent degradation of a casein protein expressed therein. As shown in the schematic, the casein accumulates in the seed at a higher level than in a seed with wildtype levels of protease expression and/or activity.

FIG. 19 is a schematic demonstrating how the properties of a seed processing composition, or a food composition comprising the same, may be improved if the composition comprises one or more casein proteins. These properties may be improved if the composition comprises a casein protein monomer (i.e., a casein protein that is not part of a fusion protein), or a fusion protein comprising one or more caseins.

FIG. 20 is a schematic demonstrating an illustrative mechanism that may be used to protect one or more proteins (e.g., casein proteins) from degradation in a host cell, leading to accumulation thereof. The protein (e.g., a casein protein) is fused to one or more proteins that is capable of forming a protein body (e.g., a prolamin). After the fusion protein is synthesized and retained in the endoplasmic reticulum (ER), a protein body is formed (PB). The fusion protein (including, for example, the casein protein) is contained within the PB. Proteases that would degrade the caseins, do not have access to the fusion protein inside the PB. In this figure, the term “PSV” refers to protein storage vacuole.

FIG. 21 shows protein detection by western blotting. The top panel shows detection of a fusion protein comprising β-casein and casein using a primary antibody raised against β-casein (B-CN). Commercial protein was detected at an apparent MW of ˜30 kDa (arrowhead; theoretical: 23.5 kDa). The fusion protein was detected at an apparent MW of ˜50 kDa (arrow; theoretical: 44.3 kDa). The first lane shows molecular weight markers. The second lane shows protein from T1 seed from recombinant plant line KV7. Lane 3-7 shows soybean wildtype seed extracts spiked with 0%, 1%, 2%, 4%, or 6% TSP commercially available β-casein. The bottom panel shows a protein gel as a control for equal lane loading, visualized using stain-free detection by Bio Rad® (image is taken at the end of the SDS run). 2.5 μg of total protein extracts were loaded per lane.

FIG. 22 shows protein detection by western blotting. The top panel shows detection of a fusion protein comprising β-casein and a partial zein (amino acids 17-112) using a primary antibody raised against β-casein (B-CN). Commercial protein was detected at an apparent MW of ˜30 kDa (arrowhead; theoretical: 23.5 kDa). The fusion protein was detected at an apparent MW of ˜30 kDa (arrow; theoretical: 23.5 kDa). The first four lanes show protein from T1 seed from a recombinant plant. The fifth lane shows molecular weight markers. Lanes 6-9 shows soybean wildtype seed extracts spiked with 0%, 1.5%, 2.5%, or 5% TSP commercially available β-casein. The bottom panel shows a protein gel as a control for equal lane loading, visualized using stain-free detection by Bio Rad® (image is taken at the end of the SDS run). 2.5 μg of total protein extracts were loaded per lane.

FIG. 23 shows a binary Agrobacterium vector used to co-express a Gene of Interest (GOI, e.g., a casein protein) and a kinase (e.g., a Fam20C kinase) in a plant cell.

FIG. 24A-FIG. 24E shows expression constructs used to co-express a Gene of Interest (GOI, e.g., a casein protein) and a kinase (e.g., a Fam20C kinase) in a plant cell.

FIG. 25A-FIG. 25F show expression constructs used to express a Gene of Interest (GOI, e.g., a casein protein) in a plant cell, wherein the GOI is fused to a glycoprotein tag, such as a (SP)11 tag.

FIG. 26A-FIG. 26G shows expression constructs used to co-express a Gene of Interest (GOI, e.g., a casein protein) and a protein capable of inducing a protein body (e.g., a prolamin, zein, canein, hydrophobin, or elastin-like protein) in a plant cell.

FIG. 27 shows a binary Agrobacterium vector used to co-express a Gene of Interest (GOI, e.g., a casein protein) and a protein capable of inducing a protein body in a plant cell.

FIG. 28 is a photograph which depicts the melting properties of various cheese compositions made with isolated kappa and beta-caseins. Top left: composition A (75% kappa-casein, 25% beta-casein); top right: composition B (100% kappa-casein); bottom left: composition C (50% kappa-casein, 50% beta-casein), bottom right: composition A (100% beta-casein).

FIG. 29 is a line graph showing cheese stretch with increasing contribution of protein from beta-casein (see also Tables 23-28).

FIG. 30 is a line graph showing melt scores of cheese compositions comprising one or more of beta-casein, kappa-casein and alpha casein (see also Tables 23-28).

FIG. 31 is a line graph showing stretch of cheese compositions comprising one or more of beta-casein, kappa-casein and alpha casein (see also Tables 23-28).

FIG. 32 is a graph showing estimated apparent viscosity (in centipoise (cP)) at shear rates in the range of 0.01 to 1000 sec⁻¹ for a milk composition comprising beta-casein as the only casein (BC milk), a yogurt composition comprising beta-casein as the only casein (BC yogurt), and an ice cream mix composition comprising beta-casein as the only casein (BC IC mix).

FIG. 33 is a western blot showing expression of a beta-casein tetramer (BC4) in E. Coli. Commercial beta-casein, in monomeric form, was detected at an apparent molecular weight of ˜30 kDA (theoretical: 23.5 kDa—arrowhead). The BC4 fusion protein was detected at an apparent MW of ˜100 kDa (theoretical: 94 kDa—arrow).

FIG. 34 is a western blot showing expression of a fusion protein comprising beta-casein and beta-lactoglobulin in tobacco leaves. Commercial beta-casein, in monomeric form, was detected at an apparent molecular weight of ˜30 kDa (theoretical: 23.5 kDa—arrowhead). The fusion protein was detected at an apparent MW of ˜48 kDa (theoretical: 42 kDa—arrow).

FIG. 35 is a graphic depicting the anthocyanin metabolic pathway in soybean.

FIG. 36A and FIG. 36B show exemplary strategies whereby a pigmentation-expressing cassette and dairy-expressing cassette are in the same T-DNA so the two traits will co-segregate.

FIG. 37A-FIG. 37C show pigmented soybeans according to the present disclosure. FIG. 37A shows an exemplary strategy employing a stable pigmented line that can be crossed with a stable high dairy-expressing line to induce pigmentation (e.g., Overexpression of MYB TFs in seed coat). Representative phenotypes of developing seeds are shown. Black arrows point to the dissected seed coat. Pigment accumulation is specific to the seed coat. FIG. 37B shows results of the same strategy except depicted are representative phenotypes of harvested seeds for the Myb factors. Pigment accumulation is specific to the seed coat. FIG. 37C shows representative phenotypes of harvested seeds generated using strategy 2 for the chalcone synthase.

FIG. 38 is a graphic of a light microscopy picture of soybean (G. max) seeds analyzed by micro-PIXE.

FIG. 39A depicts a AR03-04 expression vector with an expression cassette comprising i) AR-Pro3 promoter, ii) AR-Pro3 signal sequence, iii) DNA insert of interest (e.g., OKC1-T: Optimized Kappa Casein Truncated version 1), iv) NOS terminator. For the targeted gene editing, the expression cassette of the AR03-12 vector further comprises i) RNA-guided nuclease gene driven by GmEf1A promoter, ii) a first guide RNA expression cassette containing a first Glycinin target sequence and iii) a second guide RNA expression cassette including a second Glycinin target sequence. This expression vector is used for dual function of i) expressing protein of interest and ii) suppressing, decreasing, and/or nullifying expression of target proteins (e.g., Glycinin 4 and Glycinin 5) in plant seeds. FIG. 39B illustrates an exemplary diagram of an expression cassette designed for stacking three transgenes, each of which encodes κ-casein protein. Three differentially codon optimized transgenes encoding κ-casein protein are driven under the control of three different seed promoters; 1) BnNap (Brassica Napin) promoter (AR-Pro 11) BconB (Beta-conglycinin B subunit) promoter (AR-Pro 13), and GmSeed2 (AR-Pro14) promoter. FIG. 39C illustrates a diagram of a total Amino Acid Rebalancing-based Method for Target Identification. The amino acid rebalancing profile is obtained by calculation of the % difference (A %) of amino acid compositions between protein of interest and plant seed of interest (total). FIG. 39D illustrates a diagram of an individual Amino Acid Rebalancing-based Method for Target Identification. The amino acid rebalancing profile is obtained by calculation of the % difference (A %) of amino acid compositions between protein of interest and highly expressed seed storage proteins in plant seed of interest (individual).

FIG. 40A-FIG. 40D show vector maps encoding exemplary fusion proteins. FIG. 40A shows a vector map comprising a nucleic acid encoding for a Beta-casein-FM-AlphaS1-casein-FM-AlphaS1-casein-FM-Beta-casein fusion protein. FIG. 40B shows a vector map comprising a nucleic acid encoding for a Beta-casein-Beta-casein-Kappa-casein-Beta-lactoglobulin fusion protein. FIG. 40C shows a vector map comprising a nucleic acid encoding for a Beta-casein-Beta-casein-Beta-casein-Beta-casein fusion protein. FIG. 40D shows a vector map comprising a nucleic acid encoding for a Gamma-Zein-Beta-casein fusion protein.

FIG. 41 shows blots comparing beta-casein fusion protein expression in E-coli vs transgenic soy seeds. Left panel: Lane 1-2: Two lanes each containing BCN, BCNx4, BCN-BCN-KCN-LG, or BCN-aS1-aS1-BCN protein expressed in E. coli after induction with IPTG. Lane 9-11: Standard of BCN commercial protein spiked in at 150, 75, and 38 ng per lane. Right Panel, lane 12-19: Two lanes each containing BCN, BCNx4, BCN-BCN-KCN-LG, or BCN-aS1-aS1-BCN protein expressed in soy seeds. Lane 20: Molecular weight markers. Detection of the fusion protein using a primary antibody raised against BCN. The BCN protein is detected at an apparent MW of −25 kDa (theoretical: 23.5 kDa—gray arrow). The BCNx4 fusion protein is detected at an apparent MW of −100 kDa (theoretical: 94 kDa—orange arrow). The BCN-BCN-KCN-LG fusion protein is detected at an apparent MW of −90 kDa (theoretical: 84.5 kDa—blue arrow). The BCN-aS1-aS1-BCN fusion protein is detected at an apparent MW of −100 kDa (theoretical: 93 kDa—yellow arrow). The BCN commercial protein is detected at an apparent MW of −30 kDa (theoretical: 23.5 kDa—black arrow). Left panel shows control for total protein loading—Stain free detection by Bio-Rad. The amount of total protein extract (μg) loaded is indicated below each lane.

FIG. 42 depicts a graph illustrating the fatty acid modulation techniques of the present disclosure. Plant-based food compositions can differ in their fatty acid profiles compared to animal-based counterparts. Blending of small chain fatty acids can modulate the fatty acid profile so that it better mimics that of the corresponding animal product. For example, soy-based buttery spread containing mammalian milk proteins and soybean oil (circle markers) can be supplemented with palm oil and coconut oil to better mimic the properties of butter (box markers). The resulting product (triangle markers) is expected to have improved organoleptic properties.

FIG. 43 shows sequences and predicted phosphorylated sites in beta-casein, kappa-casein, OaS1, and OsS2.

FIG. 44A and FIG. 44B show food compositions according to the present disclosure. FIG. 44A shows a graphic of a pizza with a mozzarella-style cheese generated according to methods of the disclosure. FIG. 44B shows a graphic of a soft cheese generated using methods of the disclosure.

FIG. 45A-FIG. 45D depict exemplary universal vector constructs according to the present disclosure.

FIG. 46A-FIG. 46G depict exemplary universal vector constructs according to the present disclosure.

FIGS. 47A and 47B show expression of universal vectors according to the present disclosure. FIG. 47A shows ELISA data of protein expression of plants transformed with exemplary constructs. FIG. 47B shows western blot data of protein expression of plants transformed with exemplary constructs.

FIG. 48A-FIG. 48J shows functional properties of various food compositions. FIG. 48A shows a combined score (sum of melt and stretch) of exemplary cheeses generated using different ratios of soy: casein. FIG. 48B shows an image of a cheese generated using alpha casein and beta casein at a soy to casein ratio of 1:1. FIG. 48C shows an image of a cheese generated using alpha casein and beta casein at a soy to casein ratio of 1:1. FIG. 48D shows an image of a cheese generated using no dairy protein at a soy to casein ratio of 0:1. FIG. 48E shows an image of a cheese generated using rennet casein at a soy to casein ratio of 1:1. FIG. 48F shows an image of a cheese generated using rennet casein at a soy to casein ratio of 3:1. FIG. 48G shows an image of a cheese generated using alpha casein and beta casein at a soy to casein ratio of 1:1. FIG. 48H shows an image of a cheese generated using alpha casein and beta casein at a soy to casein ratio of 3:1. FIG. 48I shows an image of a cheese generated using rennet casein at a soy to casein ratio of 1:1. FIG. 48J shows an image of a cheese generated using rennet casein at a soy to casein ratio of 3:1.

### DETAILED DESCRIPTION OF THE DISCLOSURE

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed

Provided herein are compositions and methods for producing milk proteins, which allow for safe, sustainable and humane production of milk proteins for commercial use, such as use in food compositions. The disclosure provides recombinant fusion proteins comprising at least first protein and a second protein, wherein at least one of the first protein and the second protein is a milk protein, or fragment thereof. The disclosure also provides methods for producing the recombinant fusions proteins, and food compositions comprising the same.

Also provided herein are alternative dairy compositions, solid phase protein-stabilized emulsions, cheese compositions, and colloidal suspensions, comprising one or more casein proteins, wherein the casein proteins are isolated or recombinant, and are selected from the group consisting of kappa-casein, para-kappa-casein, beta-casein, alpha-S1-casein, and alpha-S2-casein. The compositions, emulsions, or suspensions may be used to produce food compositions that have organoleptic properties similar to traditional dairy compositions.

The following description includes information that may be useful in understanding the present disclosure. It is not an admission that any of the information provided herein is prior art or relevant to the presently claimed disclosures, or that any publication specifically or implicitly referenced is prior art.

### Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art.

Any ranges listed herein are intended to be inclusive of endpoints. For example, a range of 2-4 includes 2 and 4.

As used herein, the singular forms “a,” “an,” and “the: include plural referents unless the content clearly dictates otherwise.

The term “about” or “approximately” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

As used herein, “mammalian milk” can refer to milk derived from any mammal, such as bovine, human, goat, sheep, camel, buffalo, water buffalo, dromedary, llama and any combination thereof. In some embodiments, a mammalian milk is a bovine milk.

As used herein, “structured” refers to those proteins having a well-defined secondary and tertiary structure, and “unstructured” refers to proteins that do not have well defined secondary and/or tertiary structures. An unstructured protein may also be described as lacking a fixed or ordered three-dimensional structure. “Disordered” and “intrinsically disordered” are synonymous with unstructured.

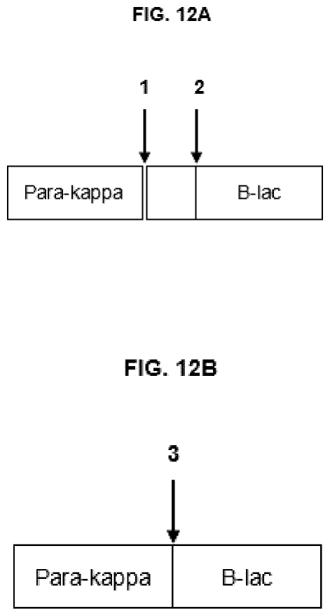

As used herein, “rennet” refers to a set of enzymes typically produced in the stomachs of ruminant mammals. Chymosin, its key component, is a protease enzyme that cleaves κ-casein (to produce para-κ-casein and a macropeptide (see e.g., FIG. 12)). In addition to chymosin, rennet contains other enzymes, such as pepsin and lipase. Rennet is used to separate milk into solid curds (for cheesemaking) and liquid whey. Rennet or rennet substitutes are used in the production of many cheeses.

As used herein “whey” refers to the liquid remaining after milk has been curdled and strained, for example during cheesemaking. Whey comprises a collection of globular proteins, typically a mixture of β-lactoglobulin, α-lactalbumin, bovine serum albumin, and immunoglobulins.

The term “plant” includes reference to whole plants, plant organs, plant tissues, and plant cells and progeny of same, but is not limited to angiosperms and gymnosperms such as Arabidopsis, potato, tomato, tobacco, alfalfa, lettuce, carrot, strawberry, sugar beet, cassava, sweet potato, soybean, lima bean, pea, chickpea, maize (corn), turf grass, wheat, rice, barley, sorghum, oat, oak, eucalyptus, walnut, palm, and duckweed as well as fern and moss. Thus, a plant may be a monocot, a dicot, a vascular plant reproduced from spores such as fern or a nonvascular plant such as moss, liverwort, hornwort, and algae. The word “plant,” as used herein, also encompasses plant cells, seeds, plant progeny, propagule whether generated sexually or asexually, and descendants of any of these, such as cuttings or seed. Plant cells include suspension cultures, callus, embryos, meristematic regions, callus tissue, leaves, roots, shoots, gametophytes, sporophytes, pollen, seeds, and microspores. Plants may be at various stages of maturity and may be grown in liquid or solid culture, or in soil or suitable media in pots, greenhouses, or fields. Expression of an introduced leader, trailer or gene sequences in plants may be transient or permanent.

The term “vascular plant” refers to a large group of plants that are defined as those land plants that have lignified tissues (the xylem) for conducting water and minerals throughout the plant and a specialized non-lignified tissue (the phloem) to conduct products of photosynthesis. Vascular plants include the clubmosses, horsetails, ferns, gymnosperms (including conifers) and angiosperms (flowering plants). Scientific names for the group include Tracheophyta and Tracheobionta. Vascular plants are distinguished by two primary characteristics. First, vascular plants have vascular tissues which distribute resources through the plant. This feature allows vascular plants to evolve to a larger size than non-vascular plants, which lack these specialized conducting tissues and are therefore restricted to relatively small sizes. Second, in vascular plants, the principal generation phase is the sporophyte, which is usually diploid with two sets of chromosomes per cell. Only the germ cells and gametophytes are haploid. By contrast, the principal generation phase in non-vascular plants is the gametophyte, which is haploid with one set of chromosomes per cell. In these plants, only the spore stalk and capsule are diploid.

The term “non-vascular plant” refers to a plant without a vascular system consisting of xylem and phloem. Many non-vascular plants have simpler tissues that are specialized for internal transport of water. For example, mosses and leafy liverworts have structures that look like leaves, but are not true leaves because they are single sheets of cells with no stomata, no internal air spaces and have no xylem or phloem. Non-vascular plants include two distantly related groups. The first group are the bryophytes, which is further categorized as three separate land plant Divisions, namely Bryophyta (mosses), Marchantiophyta (liverworts), and Anthocerotophyta (hornworts). In all bryophytes, the primary plants are the haploid gametophytes, with the only diploid portion being the attached sporophyte, consisting of a stalk and sporangium. Because these plants lack lignified water-conducting tissues, they can't become as tall as most vascular plants. The second group is the algae, especially the green algae, which consists of several unrelated groups. Only those groups of algae included in the Viridiplantae are still considered relatives of land plants.

The term “plant part” refers to any part of a plant including but not limited to the embryo, shoot, root, stem, seed, stipule, leaf, petal, flower bud, flower, ovule, bract, trichome, branch, petiole, intemode, bark, pubescence, tiller, rhizome, frond, blade, ovule, pollen, stamen, and the like. The two main parts of plants grown in some sort of media, such as soil or vermiculite, are often referred to as the “above-ground” part, also often referred to as the “shoots”, and the “below-ground” part, also often referred to as the “roots”.

The term “plant tissue” refers to any part of a plant, such as a plant organ. Examples of plant organs include, but are not limited to the leaf, stem, root, tuber, seed, branch, pubescence, nodule, leaf axil, flower, pollen, stamen, pistil, petal, peduncle, stalk, stigma, style, bract, fruit, trunk, carpel, sepal, anther, ovule, pedicel, needle, cone, rhizome, stolon, shoot, pericarp, endosperm, placenta, berry, stamen, and leaf sheath.

The term “seed” is meant to encompass the whole seed and/or all seed components, including, for example, the coleoptile and leaves, radicle and coleorhiza, scutellum, starchy endosperm, aleurone layer, pericarp and/or testa, either during seed maturation and seed germination.

“Microorganism” and “microbe” mean any microscopic unicellular organism and can include bacteria, algae, yeast, or fungi.

The term “transgenic” means an organism that has been transformed with one or more exogenous nucleic acids from another species. “Transformation” refers to a process by which a nucleic acid is introduced into a cell, either transiently or stably. Transformation may rely on any known method for the insertion of nucleic acid sequences into a prokaryotic or eukaryotic host cell, including Agrobacterium-mediated transformation protocols, viral infection, whiskers, electroporation, heat shock, lipofection, polyethylene glycol treatment, micro-injection, and particle bombardment.

“Stably integrated” refers to the permanent, or non-transient retention and/or expression of a polynucleotide in and by a cell genome. Thus, a stably integrated polynucleotide is one that is a fixture within a transformed cell genome and can be replicated and propagated through successive progeny of the cell or resultant transformed plant. Transformation may occur under natural or artificial conditions using various methods well known in the art. Transformation may rely on any known method for the insertion of nucleic acid sequences into a prokaryotic or eukaryotic host cell, including Agrobacterium-mediated transformation protocols, viral infection, whiskers, electroporation, heat shock, lipofection, polyethylene glycol treatment, micro-injection, and particle bombardment.

As used herein, the terms “stably expressed” or “stable expression” refer to expression and accumulation of a protein in a plant cell. In some embodiments, a protein may accumulate because it is not degraded by endogenous plant proteases. In some embodiments, a protein is considered to be stably expressed in a plant if it is present in the plant in an amount of 1% or higher per total protein weight of soluble protein extractable from the plant.

As used herein, the term “fusion protein” refers to a protein comprising at least two constituent proteins (or fragments or variants thereof, as defined below) that are encoded by separate genes, and that have been joined so that they are transcribed and translated as a single polypeptide. In some embodiments, a fusion protein may be separated into its constituent proteins, for example by cleavage with a protease.

The term “recombinant” refers to nucleic acids or proteins formed by laboratory methods of genetic recombination (e.g., molecular cloning) to bring together genetic material from multiple sources, creating sequences that would not otherwise be found in the genome. A recombinant fusion protein is a protein created by combining sequences encoding two or more constituent proteins, such that they are expressed as a single polypeptide. Recombinant fusion proteins may be expressed in vivo in various types of host cells, including plant cells, bacterial cells, fungal cells, mammalian cells, etc. Recombinant fusion proteins may also be generated in vitro.

The term “promoter” or a “transcription regulatory region” refers to nucleic acid sequences that influence and/or promote initiation of transcription. Promoters are typically considered to include regulatory regions, such as enhancer or inducer elements. The promoter will generally be appropriate to the host cell in which the target gene is being expressed. The promoter, together with other transcriptional and translational regulatory nucleic acid sequences (also termed “control sequences”), is necessary to express any given gene. In general, the transcriptional and translational regulatory sequences include, but are not limited to, promoter sequences, ribosomal binding sites, transcriptional start and stop sequences, translational start and stop sequences, and enhancer or activator sequences.

The term signal peptide—also known as “signal sequence”, “targeting signal”, “localization signal”, “localization sequence”, “transit peptide”, “leader sequence”, or “leader peptide”, is used herein to refer to an N-terminal peptide which directs a newly synthesized protein to a specific cellular location or pathway. Signal peptides are often cleaved from a protein during translation or transport and are therefore not typically present in a mature protein.

The term “proteolysis” or “proteolytic” or “proteolyze” means the breakdown of proteins into smaller polypeptides or amino acids. Uncatalyzed hydrolysis of peptide bonds is extremely slow. Proteolysis is typically catalyzed by cellular enzymes called proteases, but may also occur by intra-molecular digestion. Low pH or high temperatures can also cause proteolysis non-enzymatically. Limited proteolysis of a polypeptide during or after translation in protein synthesis often occurs for many proteins. This may involve removal of the N-terminal methionine, signal peptide, and/or the conversion of an inactive or non-functional protein to an active one.

The term “2A peptide”, used herein, refers to nucleic acid sequence encoding a 2A peptide or the 2A peptide itself. The average length of 2A peptides is 18-22 amino acids. The designation “2A” refers to a specific region of picornavirus polyproteins and arose from a systematic nomenclature adopted by researchers. In foot-and-mouth disease virus (FMDV), a member of Picornaviridae family, a 2A sequence appears to have the unique capability to mediate cleavage at its own C-terminus by an apparently enzyme-independent, novel type of reaction. This sequence can also mediate cleavage in a heterologous protein context in a range of eukaryotic expression systems. The 2A sequence is inserted between two genes of interest, maintaining a single open reading frame. Efficient cleavage of the polyprotein can lead to co-ordinate expression of active two proteins of interest. Self-processing polyproteins using the FMDV 2A sequence could therefore provide a system for ensuring coordinated, stable expression of multiple introduced proteins in cells including plant cells.

The term “purifying” is used interchangeably with the term “isolating” and generally refers to the separation of a particular component from other components of the environment in which it was found or produced. For example, purifying a recombinant protein from plant cells in which it was produced typically means subjecting transgenic protein containing plant material to biochemical purification and/or column chromatography.

When referring to expression of a protein in a specific amount per the total protein weight of the soluble protein extractable from the plant (“TSP”), it is meant an amount of a protein of interest relative to the total amount of protein that may reasonably be extracted from a plant using standard methods. The weight assigned to a fusion protein within total soluble protein fraction is only the weight corresponding to the referenced protein. A protein fraction comprising 2 grams of a casein-casein fusion within 100 grams TSP, the casein will be considered to represent 2% of TSP. Weights for fusions comprising different proteins will be measured by multiplying the weight of the total fusion protein by the percent of amino acids corresponding to that protein within the fusion. Thus, a protein fraction comprising 10 grams of a fusion protein (comprised of a 60 AA first protein fused to a 40 AA second protein) within a 100-gram TSP, the wt % content of the first protein would be 6%, and the wt % content of the second protein would be 4%. Amino acids corresponding to portions of the fusion that cannot be assigned any of the fusion monomers (e.g., linker sequence), are not counted in wt % calculations. Methods for extracting total protein from a plant are known in the art. For example, total protein may be extracted from seeds by bead beating seeds at about 15000 rpm for about 1 min. The resulting powder may then be resuspended in an appropriate buffer (e.g., 50 mM Carbonate-Bicarbonate pH 10.8, 1 mM DTT, 1× Protease Inhibitor Cocktail). After the resuspended powder is incubated at about 4° C. for about 15 minutes, the supernatant may be collected after centrifuging (e.g., at 4000 g, 20 min, 4° C.). Total protein may be measured using standard assays, such as a Bradford assay. The amount of protein of interest may be measured using methods known in the art, such as an ELISA or a Western Blot.

Sections of this disclosure refer to ratios between a first and second protein (e.g., a ratio by weight). When calculating ratios involving fusion proteins, the weight assigned to an individual protein within the fusion corresponds to percent of amino acids corresponding to that individual protein within the fusion. For example, the ratio between 10 grams of first protein and 10 grams of second protein, wherein the first protein is a casein-casein fusion, would be 1:1, because 100% of the fusion weight would be assigned to casein. In contrast, if the fusion protein is between an A-B fusion in which protein A was 50 AA and protein B was 50AA, then the ratio of protein A to the second protein would be 1:2, and the ratio of protein B to the second protein would be 1:2. Amino acids corresponding to portions of the fusion that cannot be assigned any of the fusion monomers (e.g., linker sequence), are not counted in these ratios.

When referring to a nucleic acid sequence or protein sequence, the term “identity” is used to denote similarity between two sequences. Unless otherwise indicated, percent identities described herein are determined using the BLAST algorithm available at the world wide web address: blast.ncbi.nlm.nih.gov/Blast.cgi using default parameters.

As used herein, the terms “dicot” or “dicotyledon” or “dicotyledonous” refer to a flowering plant whose embryos have two seed leaves or cotyledons. Examples of dicots include, but are not limited to, Arabidopsis, tobacco, tomato, potato, sweet potato, cassava, alfalfa, lima bean, pea, chickpea, soybean, carrot, strawberry, lettuce, oak, maple, walnut, rose, mint, squash, daisy, Quinoa, buckwheat, mung bean, cow pea, lentil, lupin, peanut, fava bean, French beans (i.e., common beans), mustard, or cactus.

The terms “monocot” or “monocotyledon” or “monocotyledonous” refer to a flowering plant whose embryos have one cotyledon or seed leaf. Examples of monocots include, but are not limited to turf grass, maize (corn), rice, oat, wheat, barley, sorghum, orchid, iris, lily, onion, palm, and duckweed.

As used herein, a “low lactose product” is any food composition considered by the FDA to be “lactose reduced”, “low lactose”, or “lactose free”.

As used herein, a “milk protein” is any protein, or fragment or variant thereof, that is typically found in one or more mammalian milks. In some embodiments, the milk proteins described herein are casein proteins, such as kappa-casein, para-kappa-casein, beta-casein, alpha-S1-casein, and alpha-S2-casein.

As used herein, a “non-milk” protein is any protein that is not typically found in any mammalian milk composition. One non-limiting example of a non-milk protein is green fluorescent protein (GFP).

As used herein, a “caseinate” is a compound derived from casein. Caseinates may be produced by adding acid to skim milk to reduce the pH to about 4.6, which causes the casein proteins to be precipitated. The resulting curd is rinsed and dried to produce acid casein. Acid casein is typically insoluble without further treatment, such as pH adjustment. Acid casein either before or after drying can be mixed with a base such as sodium hydroxide to produce sodium caseinate, or calcium hydroxide to produce calcium caseinate.

As used herein, an “alternative dairy composition” is a composition that comprises an isolated, or recombinant, casein protein, and may also comprise variations of the composition, such as a low-fat alternative dairy composition.

As used herein, the phrase “solid phase, protein-stabilized emulsion” refers to a homogenous and stable emulsion that is a solid at room temperature. The solid-phase, protein stabilized emulsions described herein is formed by the protein reducing the interfacial tension between the continuous aqueous phase and discontinuous lipid phase by aligning and/or unfolding at the interface. The amphiphilic nature of proteins allows them to interact with both phases and association between proteins in the aqueous phase results in decreased mobility of water in the form of increased viscosity and/or solid like behavior at different temperatures. The presence of “emulsifying salts” can enhance the emulsifying properties of the proteins.

As used herein, “cheese” refers to a food that is produced by curdling animal-derived milk. The milk may be curdled using, for example, enzymes (e.g., rennet), or using acid.

As used herein, “cheese composition” refers to a food that is produced by combining one or more milk proteins, optionally with other ingredients, as described herein. For example, cheese compositions may be produced using one or more recombinant milk proteins, or one or more milk proteins isolated from bovine milk. The cheese compositions may, in some embodiments, include only one milk protein. In some embodiments, the cheese compositions may comprise 2, 3, or 4 milk proteins. In some embodiments, the cheese compositions may comprise one or more milk proteins in a ratio that does not occur in the milk produced by any mammal (i.e., a non-naturally occurring ratio).

As used herein, the term “melt”, “melting”, or “meltability” refers to the liquefaction of cheese or a cheese composition by heat.

As used herein, the term “viscosity” or “flow” refers to the tendency of cheese (or a cheese composition) to spread and flow when completely melted.

As used herein, the term “stretch”, “stretching”, or “stretchability” refers to the formation of fibrous strands of cheese (or a cheese composition) that elongate without breaking.

As used herein, the term “oiling-off” refers to the tendency of free oil separation from melted cheese or a cheese composition (also known as fat leakage).

As used herein, the term “browning” or “blistering” refers to the trapped pockets of heated air and steam that may be scorched during baking with cheese (or a cheese composition).

As used herein, the term “whitening” or “decolorization” refers to the bleaching of cheese (or a cheese composition).

As used herein, the term “spread”, “spreading” or “spreadability” refers to the ability of cheese or a cheese composition to spread over a surface on application of slight force to form a layer, thin enough to form a coating.

The term “ash” is used herein as it is well known in the art, and means one or more ions, elements, minerals and/or compounds that may be found in mammalian produced milk. Ash may comprise one or more of sodium, potassium, calcium, magnesium, phosphorus, iron, copper, zinc, chloride, manganese, selenium, iodine, phosphate, citrate, sulfate, and carbonate. In some embodiments, ash may comprise calcium carbonate and/or sodium citrate.

### Milk Proteins

The fusion proteins described herein may comprise one or more milk proteins. In some embodiments, the fusion proteins described herein may comprise a first protein and a second protein, wherein the first protein and/or second protein is a milk protein. In some embodiments, the first protein and the second protein are both milk proteins. As used herein the term “milk protein” refers to any protein, that is typically found in one or more mammalian milks. In some embodiments, “milk protein” encompasses fragments of milk proteins lacking the signal peptide, as defined in this disclosure. Thus, in some embodiments, the term “milk protein” refers to the “mature” protein that is present in milk. Examples of mammalian milk include, but are not limited to, milk produced by a cow, human, goat, sheep, camel, horse, donkey, dog, cat, elephant, monkey, mouse, rat, hamster, guinea pig, whale, dolphin, seal, sheep, buffalo, water buffalo, dromedary, llama, yak, zebu, reindeer, mole, otter, weasel, wolf, raccoon, walrus, polar bear, rabbit, or giraffe. Some representative examples of milk protein species of the disclosure can be found in Table 34.

The composition of milk varies depending on the mammal. For example, as shown below in Table 1, cow milk comprises β-lactoglobulin, α-S1-casein, and α-S2-casein, whereas human milk does not. However, for the purposes of this disclosure, β-lactoglobulin, α-S1-casein, and α-S2-casein are considered milk proteins.

TABLE 1
Protein composition of human and cow milk

| Protein | Human milk (mg/mL) | Bovine (cow) milk (mg/mL) |
|---|---|---|
| α-lactalbumin | 2.2 | 1.2 |
| α-s1-casein | 0 | 11.6 |
| α-s2-casein | 0 | 3.0 |
| β-casein | 2.2 | 9.6 |
| κ-casein | 0.4 | 3.6 |
| γ-casein | 0 | 1.6 |
| Immunoglobulins | 0.8 | 0.6 |
| Lactoferrin | 1.4 | 0.3 |
| β-lactoglobulin | 0 | 3.0 |
| Lysozyme | 0.5 | Traces |
| Serum albumin | 0.4 | 0.4 |
| Other | 0.8 | 0.6 |

Illustrative milk proteins that may be used in the fusion proteins of the disclosure include, but are not limited to, α-S1 casein, α-S2 casein, β-casein, κ-casein, para-κ-casein, β-lactoglobulin, α-lactalbumin, lysozyme, lactoferrin, lactoperoxidase, serum albumin, and immunoglobulins (e.g., IgA, IgG, IgM, IgE).

Milk proteins may be further classified as structured or unstructured proteins. An “unstructured milk protein” is a milk protein that lacks a defined secondary structure, a defined tertiary structure, or a defined secondary and tertiary structure. Whether a milk protein is unstructured may be determined using a variety of biophysical and biochemical methods known in the art, such as small angle X-ray scattering, Raman optical activity, circular dichroism, nuclear magnetic resonance (NMR) and protease sensitivity. In some embodiments, a milk protein is considered to be unstructured if it is unable to be crystallized using standard techniques.

Illustrative unstructured milk proteins that may be used in the fusion proteins of the disclosure includes members of the casein family of proteins, such as α-S1 casein, α-S2 casein, β-casein, and κ-casein. The caseins are phosphoproteins and make up approximately 80% of the protein content in bovine milk and about 20-45% of the protein in human milk. Caseins form a multi-molecular, granular structure called a casein micelle in which some enzymes, water, and salts, such as calcium and phosphorous, are present. The micellar structure of casein in milk is significant in terms of a mode of digestion of milk in the stomach and intestine and a basis for separating some proteins and other components from cow milk. In practice, casein proteins in bovine milk can be separated from whey proteins by acid precipitation of caseins, by breaking the micellar structure by partial hydrolysis of the protein molecules with proteolytic enzymes, or microfiltration to separate the smaller soluble whey proteins from the larger casein micelle. Caseins are relatively hydrophobic, making them poorly soluble in water.

In some embodiments, the casein proteins described herein (e.g., α-S1 casein, α-S2 casein, β-casein, and/or κ-casein) are isolated or derived from cow (Bos taurus), goat (Capra hircus), sheep (Ovis aries), water buffalo (Bubalus bubalis), dromedary camel (Camelus dromedaries), bactrian camel (Camelus bactrianus), wild yak (Bos mutus), horse (Equus caballus), donkey (Equus asinus), reindeer (Rangifer tarandus), eurasian elk (Alces alces), alpaca (Vicugna pacos), zebu (Bos indicus), llama (Lama glama), or human (Homo sapiens). In some embodiments, a casein protein (e.g., α-S1 casein, α-S2 casein, β-casein, or κ-casein) has at least 80%, at least 85%, at least 90%, at least 95%, at least 96%, at least 97%, at least 98%, or at least 99% identity with a casein protein from one or more of cow (Bos taurus), goat (Capra hircus), sheep (Ovis aries), water buffalo (Bubalus bubalis), dromedary camel (Camelus dromedaries), bactrian camel (Camelus bactrianus), wild yak (Bos mutus), horse (Equus caballus), donkey (Equus asinus), reindeer (Rangifer tarandus), eurasian elk (Alces alces), alpaca (Vicugna pacos), zebu (Bos indicus), llama (Lama glama), or human (Homo sapiens).

As used herein, the term “α-S1 casein” refers to not only the α-S1 casein protein, but also fragments lacking the signal peptide, as defined above. The term fragment also includes proteins lacking up to three amino acids on the N- and/or C-terminus compared to a mature protein present in mammalian milk. α-S1 casein is found in the milk of numerous different mammalian species, including cow, goat, and sheep. The sequence, structure, and physical/chemical properties of α-S1 casein derived from various species is highly variable. An illustrative sequence for bovine α-S1 casein can be found at Uniprot Accession No. P02662, and an illustrative sequence for goat α-S1 casein can be found at GenBank Accession No. X59836.1. The terms “α-S1 casein” and “alpha-S1-casein” (and similar terms) are used interchangeably herein.

As used herein, the term “α-S2 casein” refers to not only the α-S2 casein protein, but also fragments lacking the signal peptide, as defined above. The term fragment also includes proteins lacking up to three amino acids on the N- and/or C-terminus compared to a mature protein present in mammalian milk. α-S2 is known as epsilon-casein in mouse, Gamma-casein in rat, and casein-A in guinea pig. The sequence, structure, and physical/chemical properties of α-S2 casein derived from various species is highly variable. An illustrative sequence for bovine α-S2 casein can be found at Uniprot Accession No. P02663, and an illustrative sequence for goat α-S2 casein can be found at Uniprot Accession No. P33049. The terms “α-S2 casein” and “alpha-S2-casein” (and similar terms) are used interchangeably herein.

As used herein, the term “β-casein” refers to not only the β-casein protein, but also fragments lacking the signal peptide, as defined above. The term fragment also includes proteins lacking up to three amino acids on the N- and/or C-terminus compared to a mature protein present in mammalian milk. For example, A1 and A2 β-casein are genetic variants of the β-casein milk protein that differ by one amino acid (at amino acid 67, A2 β-casein has a proline, whereas A1 has a histidine). Other genetic variants of β-casein include the A3, B, C, D, E, F, H1, H2, I and G genetic variants. The sequence, structure and physical/chemical properties of β-casein derived from various species is highly variable. Exemplary sequences for bovine β-casein can be found at Uniprot Accession No. P02666 and GenBank Accession No. M15132.1. The terms “β-casein”, “beta-casein” and “B-casein” (and similar terms) are used interchangeably herein.

As used herein, the term “κ-casein” refers to not only the κ-casein protein, but also fragments lacking the signal peptide, as defined above. The term fragment also includes proteins lacking up to three amino acids on the N- and/or C-terminus compared to a mature protein present in mammalian milk. κ-casein is cleaved by rennet, which releases a macropeptide from the C-terminal region. The remaining product with the N-terminus and approximately two-thirds of the original peptide chain is referred to as para-κ-casein. The sequence, structure and physical/chemical properties of κ-casein derived from various species is highly variable. Illustrative sequences for bovine κ-casein can be found at Uniprot Accession No. P02668 and GenBank Accession No. CAA25231. The terms “κ-casein”, “k-casein” and “kappa-casein” (and similar terms) are used interchangeably herein.

In some embodiments, the milk protein is a casein protein, for example, α-S1 casein, α-S2 casein, β-casein, and or κ-casein. In some embodiments, the milk protein is κ-casein and comprises the sequence of SEQ ID NO: 4, or a sequence at least 90%, at least 95%, at least 96%, at least 97%, at least 98%, or at least 99% identical thereto. In some embodiments, the milk protein is para-κ-casein and comprises the sequence of SEQ ID NO: 2, or a sequence at least 90%, at least 95%, at least 96%, at least 97%, at least 98%, or at least 99% identical thereto. In some embodiments, the milk protein is β-casein and comprises the sequence of SEQ ID NO: 6, or a sequence at least 90%, at least 95%, at least 96%, at least 97%, at least 98%, or at least 99% identical thereto. In some embodiments, the milk protein is α-S1 casein and comprises the sequence SEQ ID NO: 8, or a sequence at least 90%, at least 95%, at least 96%, at least 97%, at least 98%, or at least 99% identical thereto. In some embodiments, milk protein is α-S2 casein and comprises the sequence SEQ ID NO: 84, or a sequence at least 90%, at least 95%, at least 96%, at least 97%, at least 98%, or at least 99% identical thereto.

In some embodiments, the milk protein comprises a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 4. In some embodiments, the milk protein comprises a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 2. In some embodiments, the milk protein comprises a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 6. In some embodiments, the milk protein comprises a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 8. In some embodiments, the milk protein comprises a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 84.

In some embodiments, α-S1 casein is encoded by the sequence of SEQ ID NO: 7, or a sequence at least 90%, at least 95%, at least 96%, at least 97%, at least 98%, or at least 99% identical thereto. In some embodiments, α-S2 casein is encoded by the sequence of SEQ ID NO: 83, or a sequence at least 90%, at least 95%, at least 96%, at least 97%, at least 98%, or at least 99% identical thereto. In some embodiments, β-casein is encoded by the sequence of SEQ ID NO: 5, or a sequence at least 90%, at least 95%, at least 96%, at least 97%, at least 98%, or at least 99% identical thereto. In some embodiments, κ-casein is encoded by the sequence of SEQ ID NO: 3, or a sequence at least 90%, at least 95%, at least 96%, at least 97%, at least 98%, or at least 99% identical thereto. In some embodiments, para-κ-casein is encoded by the sequence of SEQ ID NO: 1, or a sequence at least 90%, at least 95%, at least 96%, at least 97%, at least 98%, or at least 99% identical thereto.

In some embodiments, the milk protein is encoded by a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 7. In some embodiments, the milk protein is encoded by a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 83. In some embodiments, the milk protein is encoded by a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 3. In some embodiments, the milk protein is encoded by a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 1. In some embodiments, the milk protein is encoded by a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 5.

In some embodiments, the milk protein is a casein protein, and comprises a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to any one of SEQ ID NO: 85-133, or 148-563. In some embodiments, the milk protein is a casein protein and comprises the sequence of any one of SEQ ID NO: 85-133 or 148-563.

In some embodiments, the milk protein comprises a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to any one of SEQ ID NO: 85-98 or 148-340. In some embodiments, the milk protein comprises the sequence of any one of SEQ ID NO: 85-98 or 148-340.

In some embodiments, the milk protein comprises a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to any one of SEQ ID NO: 99-109 or 341-440. In some embodiments, the milk protein comprises the sequence of any one of SEQ ID NO: 99-109 or 341-440.

In some embodiments, the milk protein comprises a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to any one of SEQ ID NO: 110-120 or 441-494. In some embodiments, the milk protein comprises the sequence of any one of SEQ ID NO: 110-120 or 441-494.

In some embodiments, the milk protein comprises a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to any one of SEQ ID NO: 121-133 or 495-563. In some embodiments, the milk protein comprises the sequence of any one of SEQ ID NO: 121-133 or 495-563 or 495-563.

In some embodiments, the milk protein is a structured protein. Examples of structured milk proteins include, for example, β-lactoglobulin, α-lactalbumin, lysozyme, lactoferrin, lactoperoxidase, serum albumin, or an immunoglobulin.

In some embodiments, the milk protein is β-lactoglobulin and comprises the sequence of SEQ ID NO: 10, or a sequence at least 90%, at least 95%, at least 96%, at least 97%, at least 98%, or at least 99% identical thereto. In some embodiments, the milk protein is β-lactoglobulin and is encoded by the sequence of any one of SEQ ID NO: 9, 11, 12, or 13, or a sequence at least 90%, at least 95%, at least 96%, at least 97%, at least 98%, or at least 99% identical to any one of SEQ ID NO: 9, 11, 12, or 13. In some embodiments, the milk protein comprises a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to any one of SEQ ID NO: 9-13 or 564-614. In some embodiments, the milk protein comprises the sequence of any one of SEQ ID NO: 10 or 564-614.

### Fusion Partners

The fusion proteins described herein comprise a first protein (also referred to as first fusion protein or first fusion partner) and a second protein (also referred to second fusion protein or second fusion partner), wherein at least one of the first protein and the second protein is a milk protein. Accordingly, in addition to the milk protein, the fusion proteins described herein comprise a “fusion partner” (i.e., the second protein)—a protein that is fused the milk protein in a fusion protein.

In some embodiments, fusion partner is a protein with a molecular weight of about 5 to about 100 kDa. For example, the fusion partner may have a molecular weight of at least 5 kDa, at least 10 kDa, at least 15 kDa, about 20 kDa, about 25 kDa, about 30 kDa, about 35 kDa, about 40 kDa, about 45 kDa, about 50 kDa, about 55 kDa, about 60 kDa, about 65 kDa, about 70 kDa, about 75 kDa, about 80 kDa, about 85 kDa, about 90 kDa, about 95 kDa, or about 100 kDa. In some embodiments, the fusion partner is a protein with a molecular weight of about 15 kDa, or more.

In some embodiments, fusion partner is a protein with about 10% to about 90% hydrophobic amino acids, e.g., about 10% to about 20%, about 20% to about 30%, about 30% to about 40%, about 40% to about 50%, about 50% to about 60%, about 60% to about 70%, about 70% to about 80%, or about 80% to about 90%. In some embodiments, the fusion partner may comprise at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% hydrophobic amino acids. In some embodiments, the fusion partner is a protein with about 25% or more hydrophobic amino acids. In some embodiments, the fusion partner is a protein with about 30% or more hydrophobic amino acids. In some embodiments, the fusion partner is a protein with about 35% or more hydrophobic amino acids. In some embodiments, the fusion partner is a protein with about 40% or more hydrophobic amino acids. A hydrophobic amino acid is an amino acid with a hydrophobic side chain, such as alanine (A), valine (V), isoleucine (I), leucine (L), methionine (M), phenylalanine (F), tryptophan (W), tyrosine (Y), or proline.

In some embodiments, the fusion partner is a flexible protein. In general, proteins with fewer disulfide bonds are more flexible. In some embodiments, the fusion partner comprises less than about 5 disulfide bonds per 10 kDa molecular weight. In some embodiments, the fusion partner comprises less than about 4.5 disulfide bonds per 10 kDa molecular weight. In some embodiments, the fusion partner comprises less than about 4.0 disulfide bonds per 10 kDa molecular weight. In some embodiments, the fusion partner comprises less than about 3.5 disulfide bonds per 10 kDa molecular weight. In some embodiments, the fusion partner comprises less than about 3.0 disulfide bonds per 10 kDa molecular weight. In some embodiments, the fusion partner comprises less than about 2.5 disulfide bonds per 10 kDa molecular weight. In some embodiments, the fusion partner comprises less than about 2.0 disulfide bonds per 10 kDa molecular weight. In some embodiments, the fusion partner comprises less than about 1.5 disulfide bonds per 10 kDa molecular weight. In some embodiments, the fusion partner comprises less than about 1 disulfide bond per 10 kDa molecular weight. Number of disulfide bonds may be predicted using one or more computer algorithms known to those of skill in the art. For example, the software SnapGene® or the Prot Pi tool (available on the Internet by placing https://in front of www.protpi.ch/Calculator) may be useful for making such predictions. Notably, as understood by those of skill in the art, the number of cysteines in a protein, on its own, is not necessarily predictive of the number of disulfide bonds in that protein. The secondary and tertiary structure of the protein must also be considered, to determine whether a given cysteine is in appropriate proximity to another cysteine in order to form a bond.

In some embodiments, the fusion partner comprises at least one of the following characteristics: (i) a molecular weight of 15 kDa or higher, (ii) at least 30% hydrophobic amino acids, (iii) less than about 2.5 disulfide bonds per 10 kDa molecular weight. In some embodiments, the fusion partner comprises at least two of the following characteristics: (i) a molecular weight of 15 kDa or higher, (ii) at least 30% hydrophobic amino acids, (iii) less than about 2.5 disulfide bonds per 10 kDa molecular weight. In some embodiments, the fusion partner comprises all three of the following characteristics: (i) a molecular weight of 15 kDa or higher, (ii) at least 30% hydrophobic amino acids, and (iii) less than about 2.5 disulfide bonds per 10 kDa molecular weight.

In some embodiments, the fusion partner comprises at least one of the following characteristics: (i) a molecular weight of 10 kDa or higher, (ii) at least 30% hydrophobic amino acids, (iii) less than about 2.5 disulfide bonds per 10 kDa molecular weight. In some embodiments, the fusion partner comprises at least one of the following characteristics: (i) a molecular weight of 11 kDa or higher, (ii) at least 30% hydrophobic amino acids, (iii) less than about 2.5 disulfide bonds per 10 kDa molecular weight. In some embodiments, the fusion partner comprises at least one of the following characteristics: (i) a molecular weight of 12 kDa or higher, (ii) at least 30% hydrophobic amino acids, (iii) less than about 2.5 disulfide bonds per 10 kDa molecular weight. In some embodiments, the fusion partner comprises at least one of the following characteristics: (i) a molecular weight of 13 kDa or higher, (ii) at least 30% hydrophobic amino acids, (iii) less than about 2.5 disulfide bonds per 10 kDa molecular weight. In some embodiments, the fusion partner comprises at least one of the following characteristics: (i) a molecular weight of 14 kDa or higher, (ii) at least 30% hydrophobic amino acids, (iii) less than about 2.5 disulfide bonds per 10 kDa molecular weight. In some embodiments, the fusion partner comprises at least one of the following characteristics: (i) a molecular weight of 15 kDa or higher, (ii) at least 30% hydrophobic amino acids, (iii) less than about 2.5 disulfide bonds per 10 kDa molecular weight. In some embodiments, the fusion partner comprises at least one of the following characteristics: (i) a molecular weight of 16 kDa or higher, (ii) at least 30% hydrophobic amino acids, (iii) less than about 2.5 disulfide bonds per 10 kDa molecular weight. In some embodiments, the fusion partner comprises at least one of the following characteristics: (i) a molecular weight of 17 kDa or higher, (ii) at least 30% hydrophobic amino acids, (iii) less than about 2.5 disulfide bonds per 10 kDa molecular weight. In some embodiments, the fusion partner comprises at least one of the following characteristics: (i) a molecular weight of 18 kDa or higher, (ii) at least 30% hydrophobic amino acids, (iii) less than about 2.5 disulfide bonds per 10 kDa molecular weight. In some embodiments, the fusion partner comprises at least one of the following characteristics: (i) a molecular weight of 19 kDa or higher, (ii) at least 30% hydrophobic amino acids, (iii) less than about 2.5 disulfide bonds per 10 kDa molecular weight. In some embodiments, the fusion partner comprises at least one of the following characteristics: (i) a molecular weight of 20 kDa or higher, (ii) at least 30% hydrophobic amino acids, (iii) less than about 2.5 disulfide bonds per 10 kDa molecular weight. In some embodiments, the fusion partner comprises at least one of the following characteristics: (i) a molecular weight of 21 kDa or higher, (ii) at least 30% hydrophobic amino acids, (iii) less than about 2.5 disulfide bonds per 10 kDa molecular weight. In some embodiments, the fusion partner comprises at least one of the following characteristics: (i) a molecular weight of 22 kDa or higher, (ii) at least 30% hydrophobic amino acids, (iii) less than about 2.5 disulfide bonds per 10 kDa molecular weight. In some embodiments, the fusion partner comprises at least one of the following characteristics: (i) a molecular weight of 23 kDa or higher, (ii) at least 30% hydrophobic amino acids, (iii) less than about 2.5 disulfide bonds per 10 kDa molecular weight. In some embodiments, the fusion partner comprises at least one of the following characteristics: (i) a molecular weight of 24 kDa or higher, (ii) at least 30% hydrophobic amino acids, (iii) less than about 2.5 disulfide bonds per 10 kDa molecular weight. In some embodiments, the fusion partner comprises at least one of the following characteristics: (i) a molecular weight of 25 kDa or higher, (ii) at least 30% hydrophobic amino acids, (iii) less than about 2.5 disulfide bonds per 10 kDa molecular weight.

In some embodiments, the fusion partner comprises a molecular weight of 15 kDa or higher and at least 30% hydrophobic amino acids. In some embodiments, the fusion partner comprises a molecular weight of 15 kDa or higher and less than about 2.5 disulfide bonds per 10 kDa molecular weight. In some embodiments, the fusion partner comprises at least 30% hydrophobic amino acids and less than about 2.5 disulfide bonds per 10 kDa molecular weight.

In some embodiments, the fusion partner is kappa-casein. In some embodiments, the fusion partner is beta-casein. In some embodiments, the fusion partner is alpha-casein. In some embodiments, the fusion partner is beta-lactoglobulin. In some embodiments, the fusion partner is green fluorescent protein. In some embodiments the fusion partner is lysozyme. In some embodiments, fusion partner is 2S globulin. In some embodiments, the fusion partner is oleosin A. In some embodiments, the fusion partner is oleosin B. In some embodiments, the fusion partner is the Kunitz-Trypsin inhibitor. In some embodiments the fusion partner is the Bowman-Birk inhibitor. In some embodiments, the fusion partner is Hydrophobin II.

### Non-Milk Proteins

In some embodiments, the fusion partner is a non-milk protein. Accordingly, in some embodiments, the fusion proteins described herein may comprise one or more non-milk proteins, including any fragment or variant thereof. As used herein, the term “non-milk protein” refers to any protein that is not typically present in any mammalian milk composition. In some embodiments, the fusion proteins described herein may comprise a first protein and a second protein, wherein the first protein is a milk protein and the second protein (i.e., the fusion partner) is a non-milk protein. The non-milk protein may be, for example, an animal protein or a plant protein. In some embodiments, the animal protein is a mammalian protein. In some embodiments, the animal protein is an avian protein. The non-milk proteins described herein may be classified as structured or unstructured. In some embodiments, the non-milk protein is a structured protein. In some embodiment, the non-milk protein is an unstructured protein.

Whether a protein is structured may be determined using a variety of biophysical and biochemical methods known in the art, such as small angle X-ray scattering, Raman optical activity, circular dichroism, and protease sensitivity. In some embodiments, a protein is considered to be structured if it has been crystallized or if it may be crystallized using standard techniques.

In some embodiments, the non-milk protein is a protein that is typically used as a marker. As used herein, the term “marker” refers to a protein that produces a visual or other signal and is used to detect successful delivery of a vector (e.g., a DNA sequence) into a cell. Proteins typically used as a marker may include, for example, fluorescent proteins (e.g., green fluorescent protein (GFP)). Other examples include yellow fluorescent protein (YFP), orange fluorescent protein, blue fluorescent protein (BFP), cyan fluorescent protein (CFP), or red fluorescent protein (RFP). Non-limiting examples of proteins within these color classes are shown below in Table 2 (See also, Schaner, N. et al., A guide to choosing fluorescent proteins, 2005, Nature, 2:12, 905-909).

TABLE 2
Examples of fluorescent proteins

| Color class | Protein |
|---|---|
| Far-red | mPlum |
| Red | mCherry |
| | tdTomato |
| | mStrawberry |
| | J-Red |
| | DsRed-monomer |
| Orange | mOrange |
| | mKO |
| Yellow-green | mCitrine |
| | Venus |
| | YPet |
| | EYFP |
| Green | Emerald |
| | EGFP |
| | GFP |
| Cyan | CyPet |
| | mCFPm |
| | Cerulean |
| UV-excitable green | T-Sapphire |

Other examples of marker proteins include, but are not limited to, bacterial or other enzymes (e.g., β-glucuronidase (GUS), β-galactosidase, luciferase, chloramphenicol acetyltransferase).

Additional non-limiting examples of non-milk proteins that may be used in the fusion proteins described herein are provided in Table 3. In some embodiments, a fragment or variant of any one of the proteins listed in Table 3 may be used.

TABLE 3
Non-milk proteins for use as fusion partners

| | Protein or | | Exemplary |
|---|---|---|---|
| | Protein | | Uniprot |
| Categories | family | Native Species | Accession No. |
| Mammalian | Collagen | Human (Homo sapiens) | Q02388, |
| | family | | P02452, |
| | | | P08123, |
| | | | P02458 |
| | Hemoglobin | Bovine (Bos taurus) | P02070 |
| Avian | Ovalbumin | Chicken (Gallus gallus) | P01012 |
| proteins | Ovotransferrin | Chicken (Gallus gallus) | P02789 |
| | Ovoglobulin | Chicken (Gallus gallus) | I0J170 |
| | Lysozyme | Chicken (Gallus gallus) | P00698 |
| Plant | Oleosins | Soybean (Glycine max) | P29530, |
| Proteins | | | P29531 |
| | Leghemoglobin | Soybean (Glycine max) | Q41219 |
| | Extensin-like | Soybean (Glycine soja) | A0A445JU93 |
| | protein family | | |
| | Prolamin | Rice (Oryza sativa) | Q0DJ45 |
| | Glutenin | Wheat (Sorghum bicolor) | P10388 |
| | Gamma-kafirin | Wheat (Sorghum bicolor) | Q41506 |
| | preprotein | | |
| | Alpha globulin | Rice (Oryza sativa) | P29835 |
| | Basic 7S | Soybean (Glycine max) | P13917 |
| | globulin | | |
| | precursor | | |
| | 2S albumin | Soybean (Glycine max) | P19594 |
| | Beta- | Soybean (Glycine max) | P0DO16, |
| | conglycinins | | P0DO15, |
| | | | P0DO15 |
| | Glycinins | Soybean (Glycine max) | P04347, |
| | | | P04776, |
| | | | P04405 |
| | Canein | Sugar cane (Saccharum | ABP64791.1 |
| | | officinarum) | |
| | Zein | Corn (Zea Mays) | ABP64791.1 |
| | Patatin | Tomato (Solanum | P07745 |
| | | lycopersicum) | |
| | Kunitz-Trypsin | Soybean (Glycine max) | Q39898 |
| | inhibitor | | |
| | Bowman-Birk | Soybean (Glycine max) | I1MQD2 |
| | inhibitor | | |
| | Cystatine | Tomato (Solanum | Q9SE07 |
| | | lycopersicum) | |
| Fungal | Hydrophobin I | Fungus (Trichoderma | P52754 |
| proteins | | reesei) | |
| | Hydrophobin II | Fungus (Trichoderma | P79073 |
| | | reesei) | |

In some embodiments, the non-milk protein may be an animal protein. For example, in some embodiments, the non-milk protein may be a mammalian protein. The mammalian protein may be, for example, hemoglobin or collagen. In some embodiments, the non-milk protein is an avian protein, such as ovalbumin, ovotransferrin, lysozyme or ovoglobulin.

In some embodiments, the non-milk protein is a plant protein. In some embodiments, the non-milk protein is a protein that is typically expressed in a seed. In some embodiments, the plant protein is a protein that is not typically expressed in a seed. In some embodiments, the plant protein is a storage protein, e.g., a protein that acts as a storage reserve for nitrogen, carbon, and/or sulfur. In some embodiments, the plant protein may inhibit one or more proteases. In some embodiments, the non-milk protein is a plant protein selected from: oleosins, leghemoglobin, extension-like protein family, prolamin, glutenin, gamma-kafirin preprotein, α-globulin, basic 7S globulin precursor, 2S albumin, β-conglycinins, glycinins, canein, zein, patatin, kunitz-trypsin inhibitor, bowman-birk inhibitor, and cystatine. Illustrative plant proteins that may be used to inhibit one or more proteases are shown below in Table 4. In some embodiments, the non-milk protein comprises the sequence of any one of SEQ ID NO: 840, 842, 844, 846, 848 or 850. In some embodiments, the non-milk protein comprises a sequence having at least 85%, at least 90%, at least 91%, at least 92%, at least 93%, at least 94%, at least 95%, at least 96%, at least 97%, at least 98%, or at least 99% identity to any one of SEQ ID NO: 840, 842, 844, 846, 848 or 850. In some embodiments, the non-milk protein comprises a sequence having the sequence ofany one of SEQ ID NO: 840, 842, 844, 846, 848 or 850 plus at least 1, at least 2, at least 3, at least 4, at least 5, at least 6, at least 7, at least 8, at least 9, at least 10, at least 11, at least 12, at least 13, at least 14, at least 15, or more amino acid substitutions.

TABLE 4
Proteins capable of inhibiting plant proteases

| | | | DNA | Protein |
|---|---|---|---|---|
| | Short | Accession No. | Sequence | Sequence |
| Protein Name | Name | (Uniprot) | (SEQ ID) | (SEQ ID) |
| Bowman-Birk | GmBBID- | Glyma16g33400 | 839 | 840 |
| serine protease | II | | | |
| inhibitor D-II | | | | |
| Bowman-Birk | GMBBI- | Glyma14g26410 | 841 | 842 |
| serine protease | A1 | | | |
| inhibitor A1 | | | | |
| Kunitz-type | GmKTi1 | Glyma01g10900 | 843 | 844 |
| trypsin inhibitor | | | | |
| gene 1 | | | | |
| Kunitz-type | GmKTi2 | AAB23483 | 845 | 846 |
| trypsin inhibitor | | | | |
| gene 2 | | | | |
| Kunitz-type | GmKTi3 | Glyma08g45531 | 847 | 848 |
| trypsin inhibitor | | | | |
| gene 3 | | | | |
| Cystatine | SICYS8 | | 849 | 850 |
| proteinase | | | | |
| inhibitor | | | | |
| (Cystatin) | | | | |

In some embodiments, the structured protein is a fungal protein. For example, the fungal protein may be selected from hydrophobin I and hydrophobin II.

### Fusion Proteins

Described herein are fusion proteins comprising at least first protein and a second protein. In some embodiments, at least one of the first protein and the second protein is a milk protein. In some embodiments, a fusion protein comprises at least two proteins, such as three, four, five, six, seven, eight, nine, or ten proteins, or more. In some embodiments, the proteins in the fusion proteins are linked via a linker. In some embodiments, the fusion proteins comprise one or more protease cleavage sites, such as one or more chymosin cleavage sites. Various illustrative embodiments of the fusion proteins of the disclosure are described in further detail below.

### Fusion Protein Comprising a Milk Protein and a Non-Milk Protein

In some embodiments, a fusion protein comprises at least first protein and a second protein, wherein at least one of the first protein and the second protein is a milk protein, and at least one of the first protein and the second protein is a non-milk protein. In some embodiments, a fusion protein comprises at least two proteins, such as three, four, five, six, seven, eight, nine, or ten proteins, or more.

In some embodiments, the first protein is a milk protein, and the second protein is a non-milk protein. In some embodiments, the non-milk protein is an avian protein. For example, the non-milk protein may be an avian protein selected from: ovalbumin, ovotransferrin, and ovoglobulin. In some embodiments, the non-milk protein is a protein capable of inhibiting one or more proteases, such as the proteins shown above in Table 4, or variants thereof.

In some embodiments, the fusion protein comprises α-S1 casein, or fragment thereof, and ovalbumin. In some embodiments, the fusion protein comprises α-S2 casein, or fragment thereof, and ovalbumin. In some embodiments, the fusion protein comprises β-casein, or fragment thereof, and ovalbumin. In some embodiments, the fusion protein comprises κ-casein, or fragment thereof, and ovalbumin. In some embodiments, the recombinant fusion protein comprises para-κ-casein, or fragment thereof, and ovalbumin.

In some embodiments, the fusion protein comprises α-S1 casein, or fragment thereof; and ovotransferrin. In some embodiments, the fusion protein comprises α-S2 casein, or fragment thereof; and ovotransferrin. In some embodiments, the fusion protein comprises (3-casein, or fragment thereof; and ovotransferrin. In some embodiments, the fusion protein comprises κ-casein, or fragment thereof; and ovotransferrin. In some embodiments, the fusion protein comprises para-κ-casein, or fragment thereof; and ovotransferrin.

In some embodiments, the fusion protein comprises α-S1 casein, or fragment thereof; and ovoglobulin. In some embodiments, the fusion protein comprises α-S2 casein, or fragment thereof; and ovoglobulin. In some embodiments, the fusion protein comprises β-casein, or fragment thereof; and ovoglobulin. In some embodiments, the fusion protein comprises κ-casein, or fragment thereof; and ovoglobulin. In some embodiments, the fusion protein comprises para-κ-casein, or fragment thereof; and ovoglobulin.

In some embodiments, the fusion protein comprises a non-milk protein that functions as a marker, such as green fluorescent protein (GFP). In some embodiments, the fusion protein comprises α-S1-casein, or fragment thereof; and GFP. In some embodiments, the fusion protein comprises α-S2-casein, or fragment thereof, and GFP. In some embodiments, the fusion protein comprises β-casein, or fragment thereof; and GFP. In some embodiments, the fusion protein comprises κ-casein, or fragment thereof, and GFP. In some embodiments, the fusion protein comprises para-κ-casein, or fragment thereof; and GFP.

In some embodiments, the fusion protein comprises a non-milk protein that is a plant protein. In some embodiments, the fusion protein comprises α-S1 casein, or fragment thereof; and a plant protein selected from the group consisting of hydrophobin I, hydrophobin II, oleosins, leghemoglobin, extension-like protein family, prolamin, glutenin, gamma-kafirin preprotein, α-globulin, basic 7S globulin precursor, 2S albumin, β-conglycinins, glycinins, canein, zein, patatin, kunitz-trypsin inhibitor, bowman-birk inhibitor, and cystatine.

In some embodiments, the fusion protein comprises α-S2-casein, or fragment thereof; and a plant protein selected from the group consisting of hydrophobin I, hydrophobin II, oleosins, leghemoglobin, extension-like protein family, prolamin, glutenin, gamma-kafirin preprotein, α-globulin, basic 7S globulin precursor, 2S albumin, β-conglycinins, glycinins, canein, zein, patatin, kunitz-trypsin inhibitor, bowman-birk inhibitor, and cystatine.

In some embodiments, the fusion protein comprises β-casein, or fragment thereof; and a plant protein selected from the group consisting of hydrophobin I, hydrophobin II, oleosins, leghemoglobin, extension-like protein family, prolamin, glutenin, gamma-kafirin preprotein, α-globulin, basic 7S globulin precursor, 2S albumin, β-conglycinins, glycinins, canein, zein, patatin, kunitz-trypsin inhibitor, bowman-birk inhibitor, and cystatine.

In some embodiments, the fusion protein comprises κ-casein, or fragment thereof; and a plant protein selected from the group consisting of hydrophobin I, hydrophobin II, oleosins, leghemoglobin, extension-like protein family, prolamin, glutenin, gamma-kafirin preprotein, α-globulin, basic 7S globulin precursor, 2S albumin, β-conglycinins, glycinins, canein, zein, patatin, kunitz-trypsin inhibitor, bowman-birk inhibitor, and cystatine.

In some embodiments, the fusion protein comprises para-κ-casein, or fragment thereof; and a plant protein selected from the group consisting of hydrophobin I, hydrophobin II, oleosins, leghemoglobin, extension-like protein family, prolamin, glutenin, gamma-kafirin preprotein, α-globulin, basic 7S globulin precursor, 2S albumin, β-conglycinins, glycinins, canein, zein, patatin, kunitz-trypsin inhibitor, bowman-birk inhibitor, and cystatine.

In some embodiments, the fusion protein comprises γ-zein and β-casein, in that respective configuration.

### Fusion Proteins Comprising a Milk Protein and an Animal (e.g., Mammalian) Protein

In some embodiments, the fusion proteins described herein comprise (i) a milk protein (which may be unstructured or structured), and (ii) an animal protein. In some embodiments, the fusion proteins described herein comprise (i) an unstructured milk protein, and (ii) a mammalian protein. In some embodiments, the fusion proteins described herein comprise (i) an unstructured milk protein, and (ii) an avian protein. In some embodiments, the fusion proteins described herein comprise (i) an unstructured milk protein, and (ii) a fungal protein.

In some embodiments, the fusion proteins comprise a milk protein, such as a casein protein. In some embodiments, the fusion protein comprises a milk protein selected from α-S1 casein, α-S2 casein, β-casein, and κ-casein. In some embodiments, the fusion protein comprises a milk protein isolated or derived from cow (Bos taurus), goat (Capra hircus), sheep (Ovis aries), water buffalo (Bubalus bubalis), dromedary camel (Camelus dromedaries), bactrian camel (Camelus bactrianus), wild yak (Bos mutus), horse (Equus caballus), donkey (Equus asinus), reindeer (Rangifer tarandus), eurasian elk (Alces alces), alpaca (Vicugna pacos), zebu (Bos indicus), llama (Lama glama), or human (Homo sapiens). In some embodiments, the fusion protein comprises a casein protein (e.g., α-S1 casein, α-S2 casein, β-casein, para-κ-casein or κ-casein) from cow (Bos taurus), goat (Capra hircus), sheep (Ovis aries), water buffalo (Bubalus bubalis), dromedary camel (Camelus dromedaries), bactrian camel (Camelus bactrianus), wild yak (Bos mutus), horse (Equus caballus), donkey (Equus asinus), reindeer (Rangifer tarandus), eurasian elk (Alces alces), alpaca (Vicugna pacos), zebu (Bos indicus), llama (Lama glama), or human (Homo sapiens).

In some embodiments, the fusion protein comprises a milk protein found in Table 34. In some embodiments, the fusion protein comprises a milk protein that is a variant of a protein found in Table 34. In some embodiments, the fusion protein comprises a casein protein as found in Table 34 and/or a variant thereof. In some embodiments, the fusion protein comprises a beta-lactoglobulin as found in Table 34 and/or a variant thereof. One of skill in the art would be able to utilize the numerous milk proteins taught in Table 34, along with their associated SEQ ID NO and/or accession number and find such other milk proteins as encompassed by the disclosure.

In some embodiments, the fusion protein comprises a milk protein that shares at least 50%, at least 55%, at least 60%, at least 65%, at least 70%, at least 75%, at least 80%, at least 85%, at least 90%, at least 95%, or at least 100% sequence identity to a protein in Table 34 and/or a variant thereof. In some embodiments, the fusion protein comprises a milk protein that shares at least from about 70% to about 100% sequence identity to a protein in Table 34 and/or a variant thereof. In some embodiments, the fusion protein comprises a milk protein that shares at least from about 80% to about 100% sequence identity to a protein in Table 34 and/or a variant thereof. In some embodiments, the fusion protein comprises a milk protein that shares at least from about 90% to about 100% sequence identity to a protein in Table 34 and/or a variant thereof. In some embodiments, the fusion protein comprises a milk protein that shares at least 50%, 55%, 60%, 65%, 70%, 75%, 80%, 85%, 90%, 95%, or 100% sequence identity with any one of SEQ ID NO: 148-614. In some embodiments, the fusion protein comprises a milk protein that comprises a sequence of any one of SEQ ID NO: 148-614.

In some embodiments, the fusion protein is α-S1 casein. In some embodiments, the α-S1 casein comprises the sequence SEQ ID NO: 8, or a sequence at least 70%, 80%, 90%, at least 95%, at least 96%, at least 97%, at least 98%, or at least 99% identical thereto. In some embodiments, the α-S1 casein comprises the sequence of any one of SEQ ID NO: 99-109, or a sequence at least 90%, at least 95%, at least 96%, at least 97%, at least 98%, or at least 99% identical thereto.

In some embodiments, the fusion protein comprises α-S2 casein. In some embodiments, the α-S2 casein comprises the sequence SEQ ID NO: 84, or a sequence at least 70%, 80%, 90%, at least 95%, at least 96%, at least 97%, at least 98%, or at least 99% identical thereto. In some embodiments, the α-S2 casein comprises the sequence of any one of SEQ ID NO: 110-120, or a sequence at least 90%, at least 95%, at least 96%, at least 97%, at least 98%, or at least 99% identical thereto.

In some embodiments, the fusion protein comprises β-casein. In some embodiments, the β-casein comprises the sequence of SEQ ID NO: 6, or a sequence at least 70%, 80%, 90%, at least 95%, at least 96%, at least 97%, at least 98%, or at least 99% identical thereto. In some embodiments, the β-casein comprises the sequence of any one of SEQ ID NO: 121-133, or a sequence at least 90%, at least 95%, at least 96%, at least 97%, at least 98%, or at least 99% identical thereto.

In some embodiments, the fusion protein comprises κ-casein. In some embodiments, the κ-casein comprises the sequence of SEQ ID NO: 4, or a sequence at least 70%, 80%, 90%, at least 95%, at least 96%, at least 97%, at least 98%, or at least 99% identical thereto. In some embodiments, the κ-casein comprises the sequence of any one of SEQ ID NO: 85-98, or a sequence at least 90%, at least 95%, at least 96%, at least 97%, at least 98%, or at least 99% identical thereto.

In some embodiments, the fusion protein comprises para-κ-casein. In some embodiments, the para-κ-casein comprises the sequence of SEQ ID NO: 2, or a sequence at least 70%, 80%, 90%, at least 95%, at least 96%, at least 97%, at least 98%, or at least 99% identical thereto.

In some embodiments, the fusion protein comprises β-lactoglobulin, α-lactalbumin, albumin, lysozyme, lactoferrin, lactoperoxidase, or an immunoglobulin (e.g., IgA, IgG, IgM, or IgE).

In some embodiments, the fusion protein comprises β-lactoglobulin. In some embodiments, the β-lactoglobulin comprises the sequence of SEQ ID NO: 10, or a sequence at least 70%, 80%, 90%, at least 95%, at least 96%, at least 97%, at least 98%, or at least 99% identical thereto.

In some embodiments, the fusion protein comprises a mammalian protein selected from hemoglobin and collagen. In some embodiments, the fusion protein comprises an avian protein selected from ovalbumin, ovotransferrin, lysozyme and ovoglobulin.

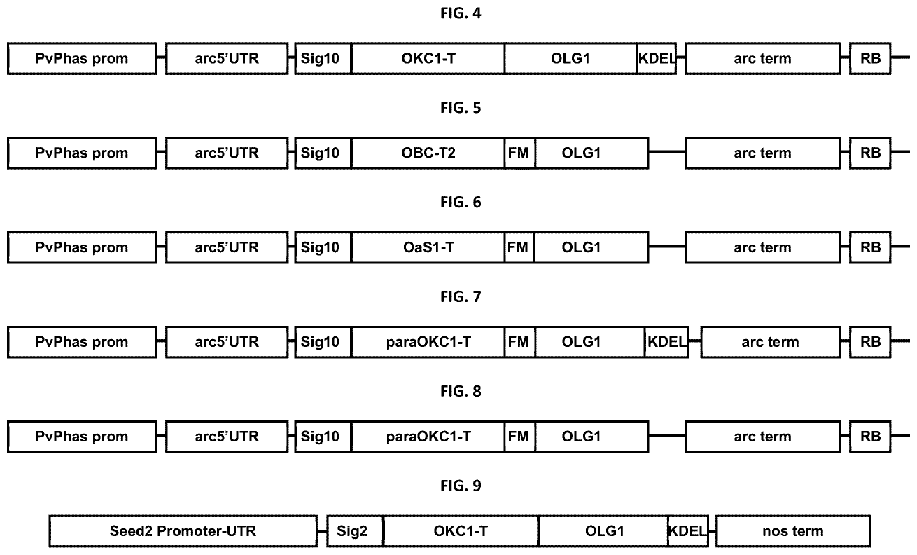

In some embodiments, a fusion protein comprises a casein protein (e.g., κ-casein, para-κ-casein, β-casein, or α-S1 casein) and β-lactoglobulin. In some embodiments, a fusion protein comprises κ-casein and β-lactoglobulin (see, e.g., FIG. 4, FIG. 9, FIG. 12A-12B). In some embodiments, a fusion protein comprises para-κ-casein and β-lactoglobulin (see, e.g., FIG. 7, FIG. 8, FIG. 12A-12B). In some embodiments, a fusion protein comprises β-casein and β-lactoglobulin. In some embodiments, a fusion protein comprises α-S1 casein and β-lactoglobulin.

In some embodiments, a plant-expressed recombinant fusion protein comprises κ-casein, or fragment thereof; and β-lactoglobulin, or fragment thereof. In some embodiments, the fusion protein comprises, in order from N-terminus to C-terminus, the κ-casein and the β-lactoglobulin.

In some embodiments, a plant-expressed recombinant fusion protein comprises β-casein, or fragment thereof; and β-lactoglobulin, or fragment thereof. In some embodiments, the fusion protein comprises, in order from N-terminus to C-terminus, the β-casein and the β-lactoglobulin.

In some embodiments, a fusion protein comprises β-casein, α-S1 casein, α-S1 casein, and β-casein, in that respective configuration. In some embodiments, a fusion protein comprises β-casein, β-casein, κ-casein, and β-lactoglobulin, in that respective configuration. In some embodiments, a fusion protein comprises β-casein, β-casein, β-casein, β-casein, in that respective configuration.

### Fusion Proteins Comprising a Milk Protein and a Plant Protein

In some embodiments, the fusion proteins described herein comprise (i) a milk protein (which may be unstructured or structured), and (ii) a plant protein. In some embodiments, the milk protein is a casein protein, such as α-S1 casein, α-S2 casein, β-casein, or κ-casein. In some embodiments, the milk protein is β-lactoglobulin, α-lactalbumin, albumin, lysozyme, lactoferrin, lactoperoxidase, or an immunoglobulin (e.g., IgA, IgG, IgM, or IgE). In some embodiments, the plant protein is selected from the group consisting of: hydrophobin I, hydrophobin II, oleosins, leghemoglobin, extension-like protein family, prolamin, glutenin, gamma-kafirin preprotein, α-globulin, basic 7S globulin precursor, 2S albumin, 0-conglycinins, glycinins, canein, zein, patatin, kunitz-trypsin inhibitor, bowman-birk inhibitor, and cystatine. In some embodiments, the plant protein is a protein that is capable of forming a protein body (PB), such as a prolamin. In some embodiments, the protein that is capable of forming a protein body comprises one or more repeat sequences, such as a repeat sequence selected from PPPPVHL (SEQ ID NO: 828); PPPPVXS, wherein X=S, Y, Q, or F (SEQ ID NO: 829); PPPV (SEQ ID NO: 830); PPVHX, wherein X=S or F (SEQ ID NO: 831); PPPVHS (SEQ ID NO: 832); PPPVXS, wherein X=Y, H, or F (SEQ ID NO: 833); PPPVXL, wherein X=H, or D (SEQ ID NO: 834); PPPVHL (SEQ ID NO: 835); PPPPPVYS (SEQ ID NO: 836); PPPPVHS (SEQ ID NO: 837); and PPPVHL (SEQ ID NO: 838). In some embodiments, the repeat sequence repeats at least 2, at least 3, at least 4, at least 5, at least 6, at least 7, at least 8, at least 9 or at least 10 times.

### Fusion Protein Comprising a Milk Protein and Prolamin

In some embodiments, the fusion protein comprises a prolamin protein, or a fragment or derivative thereof. Prolamins are a group of plant storage proteins having a high proline and glutamine amino acid content and have poor solubility in water. They are found in plants, mainly in the seeds of cereal grants such as wheat (e.g., the gliadin class of proteins), barley (e.g., the hordein class of proteins), rye (e.g., the secalin class of proteins), corn (e.g., the zein class of proteins), sorghum (e.g., the kafirin class of proteins), and oats (e.g., the avenin class of proteins).

In some embodiments, a fusion protein comprises a canein, such as a gamma canein. For example, the canein may be a 27 kD gamma canein (gCan27), or a fragment or derivative thereof gCan27 is a zein-like protein, known to be resident in the endoplasmic reticulum. An illustrative sequence for gCAN27 from sugar cane (Saccharum officinarum) can be found at Uniprot Ref. No. ABP64791.1 (SEQ ID NO: 800).

In some embodiments, the fusion protein comprises a canein, wherein the canein has the sequence of SEQ ID NO: 800, or a sequence at least 80%, at least 85%, at least 90%, at least 91%, at least 92%, at least 93%, at least 94%, at least 95%, at least 96%, at least 97%, at least 98%, or at least 99% identical thereto. In some embodiments, the fusion protein comprises a canein, wherein the canein has the sequence of SEQ ID NO: 800 with 1-5, 5-10, 10-20, 20-30, or 30-50 amino acid substitutions relative thereto. In some embodiments, the fusion protein comprises a canein, wherein the canein has a sequence corresponding to amino acids 42-237 of SEQ ID NO: 800, or a sequence at least 80%, at least 85%, at least 90%, at least 91%, at least 92%, at least 93%, at least 94%, at least 95%, at least 96%, at least 97%, at least 98%, or at least 99% identical thereto. In some embodiments, the fusion protein comprises a canein, wherein the canein has a sequence corresponding to amino acids 42-237 of SEQ ID NO: 800 with 1-5, 5-10, 10-20, 20-30, or 30-50 amino acid substitutions relative thereto. In some embodiments, the fusion protein comprises a canein, wherein the canein has the sequence of SEQ ID NO: 805, or a sequence at least 80%, at least 85%, at least 90%, at least 91%, at least 92%, at least 93%, at least 94%, at least 95%, at least 96%, at least 97%, at least 98%, or at least 99% identical thereto. In some embodiments, the fusion protein comprises a canein, wherein the canein has the sequence of SEQ ID NO: 805 with 1-5, 5-10, 10-20, 20-30, or 30-50 amino acid substitutions relative thereto. In some embodiments, the canein is encoded by the DNA sequence of SEQ ID NO: 804.

In some embodiments, the fusion protein comprises a milk protein and canein, or a fragment thereof. In some embodiments, the fusion protein comprises a casein protein and canein, or a fragment thereof. In some embodiments, the fusion protein comprises α-S1 casein and canein. In some embodiments, the fusion protein comprises α-S2-casein and canein. In some embodiments, the fusion protein comprises β-casein and canein. In some embodiments, the fusion protein comprises κ-casein and canein. In some embodiments, the fusion protein comprises para-κ-casein and canein. In some embodiments, the fusion protein comprises β-lactoglobulin and canein. In some embodiments, the fusion protein comprises the sequence of SEQ ID NO: 803, or a sequence at least 80%, at least 85%, at least 90%, at least 91%, at least 92%, at least 93%, at least 94%, at least 95%, at least 96%, at least 97%, at least 98%, or at least 99% identical thereto. In some embodiments, the fusion protein comprises the sequence of SEQ ID NO: 803, or a sequence with 1-5, 5-10, 10-20, 20-30, or 30-50 amino acid substitutions relative thereto. In some embodiments, the fusion protein is encoded by the DNA sequence of SEQ ID NO: 802.

In some embodiments, the fusion protein comprises a zein, such as gamma zein (γZein or glutenin 2). Zein is a storage protein of the prolamin class. It is found in the seeds of cereal plants and is able to accumulate within the endoplasmic reticulum (ER). In maize, for example, there are our classes of zeins (α, β, δ, γ). During endosperm development, γ- and (3-zeins are synthesized first, forming a polymer termed protein bodies (PBs) where α- and 6-zein will later accumulate (Mainieri et al, 2018). Proteins in the ER lumen usually have a tetrapeptide at the C terminus (KDEL or variations), which is necessary and sufficient for ER localization; however, zeins do not have this signal. The interactions that retain zeins in the ER are not well understood, but γ-zein is able to form ER-located PBs when expressed in storage (Coleman et al., 1996) or vegetative (Geli et al., 1994, Torrent et al., 2009, Marques et al 2020) tissues of transgenic plants in the absence of its partner zein subunits, indicating that no tissue-specific helper factors are required.

The γ-zein sequence (including the 27 kDa form of the protein) contains a signal peptide for translocation to the ER (co-translationally removed) followed by a region containing eight repeats of the hexapeptide PPPVHL (SEQ ID NO: 812), the prox domain and seven Cys residues involved in inter-chain bonds that make the protein insoluble in non-reducing conditions, and finally a second region (C-term) homologous to 2S albumins, which are vacuolar storage proteins present in various amounts in all land plants.

An illustrative sequence for γ-zein from corn (Zea mays) can be found at Uniprot Ref. No. P04706 (SEQ ID NO: 801). In some embodiments, the fusion protein comprises γ-zein, wherein the γ-zein has the sequence of SEQ ID NO: 801, or a sequence at least 80%, at least 85%, at least 90%, at least 91%, at least 92%, at least 93%, at least 94%, at least 95%, at least 96%, at least 97%, at least 98%, or at least 99% identical thereto. In some embodiments, the fusion protein comprises a γ-zein, wherein for γ-zein has the sequence of SEQ ID NO: 801 with 1-5, 5-10, 10-20, 20-30, or 30-50 amino acid substitutions relative thereto. In some embodiments, the fusion protein comprises γ-zein, wherein the γ-zein has a sequence corresponding to amino acids 17-112 of SEQ ID NO: 801, or a sequence at least 80%, at least 85%, at least 90%, at least 91%, at least 92%, at least 93%, at least 94%, at least 95%, at least 96%, at least 97%, at least 98%, or at least 99% identical thereto. In some embodiments, the fusion protein comprises γ-zein, wherein the γ-zein has a sequence corresponding to amino acids 17-112 of SEQ ID NO: 801 with 1-5, 5-10, 10-20, 20-30, or 30-50 amino acid substitutions relative thereto. In some embodiments, the fusion protein comprises a γ-zein, wherein the γ-zein has a sequence corresponding to amino acids 20-223 of SEQ ID NO: 801, or a sequence at least 80%, at least 85%, at least 90%, at least 91%, at least 92%, at least 93%, at least 94%, at least 95%, at least 96%, at least 97%, at least 98%, or at least 99% identical thereto. In some embodiments, the fusion protein comprises a γ-zein, wherein the γ-zein has a sequence corresponding to amino acids 20-223 of SEQ ID NO: 801 with 1-5, 5-10, 10-20, 20-30, or 30-50 amino acid substitutions relative thereto. In some embodiments, the fusion protein comprises a γ-zein, wherein the γ-zein has the sequence of SEQ ID NO: 809, or a sequence at least 80%, at least 85%, at least 90%, at least 91%, at least 92%, at least 93%, at least 94%, at least 95%, at least 96%, at least 97%, at least 98%, or at least 99% identical thereto. In some embodiments, the fusion protein comprises a γ-zein, wherein the γ-zein has the sequence of SEQ ID NO: 809 with 1-5, 5-10, 10-20, 20-30, or 30-50 amino acid substitutions relative thereto. In some embodiments, the γ-zein is encoded by the DNA sequence of SEQ ID NO: 808. In some embodiments, the fusion protein comprises a γ-zein, wherein the γ-zein has the sequence of SEQ ID NO: 811, or a sequence at least 80%, at least 85%, at least 90%, at least 91%, at least 92%, at least 93%, at least 94%, at least 95%, at least 96%, at least 97%, at least 98%, or at least 99% identical thereto. In some embodiments, the fusion protein comprises a γ-zein, wherein the γ-zein has the sequence of SEQ ID NO: 811 with 1-5, 5-10, 10-20, 20-30, or 30-50 amino acid substitutions relative thereto. In some embodiments, the γ-zein is encoded by the DNA sequence of SEQ ID NO: 810.

In some embodiments, the fusion protein comprises a milk protein and γ-zein, or a fragment thereof. In some embodiments, the fusion protein comprises a casein protein and γ-zein, or a fragment thereof. In some embodiments, the fusion protein comprises α-S1 casein and γ-zein. In some embodiments, the fusion protein comprises α-S2-casein and γ-zein. In some embodiments, the fusion protein comprises β-casein and γ-zein. In some embodiments, the fusion protein comprises κ-casein and γ-zein. In some embodiments, the fusion protein comprises para-κ-casein and γ-zein. In some embodiments, the fusion protein comprises β-lactoglobulin and γ-zein. In some embodiments, the fusion protein comprises the sequence of SEQ ID NO: 807, or a sequence at least 80%, at least 85%, at least 90%, at least 91%, at least 92%, at least 93%, at least 94%, at least 95%, at least 96%, at least 97%, at least 98%, or at least 99% identical thereto. In some embodiments, the fusion protein comprises the sequence of SEQ ID NO: 807, or a sequence with 1-5, 5-10, 10-20, 20-30, or 30-50 amino acid substitutions relative thereto. In some embodiments, the fusion protein is encoded by the DNA sequence of SEQ ID NO: 806.

### Fusion Protein Comprising Two or More Milk Proteins

In some embodiments, the fusion proteins described herein comprise at least first protein and a second protein, wherein the first protein and/or second protein is a milk protein. In some embodiments, the first protein and the second protein are milk proteins. In some embodiments, each of the first protein and the second protein are independently selected from α-S1 casein, α-S2 casein, β-casein, κ-casein, para-κ-casein, β-lactoglobulin, α-lactalbumin, lysozyme, lactoferrin, lactoperoxidase, serum albumin, and immunoglobulins.

In some embodiments, the recombinant fusion protein comprises α-S1 casein, or fragment thereof; and β-lactoglobulin. In some embodiments, the recombinant fusion protein comprises α-S2 casein, or fragment thereof; and β-lactoglobulin. In some embodiments, the recombinant fusion protein comprises β-casein, or fragment thereof; and β-lactoglobulin. In some embodiments, the recombinant fusion protein comprises κ-casein, or fragment thereof; and β-lactoglobulin. In some embodiments, the recombinant fusion protein comprises para-κ-casein, or fragment thereof; and β-lactoglobulin.

In some embodiments, the recombinant fusion protein comprises α-S1 casein, or fragment thereof; and α-lactalbumin. In some embodiments, the recombinant fusion protein comprises α-S2 casein, or fragment thereof; and α-lactalbumin. In some embodiments, the recombinant fusion protein comprises β-casein, or fragment thereof; and α-lactalbumin. In some embodiments, the recombinant fusion protein comprises κ-casein, or fragment thereof; and α-lactalbumin. In some embodiments, the recombinant fusion protein comprises para-κ-casein, or fragment thereof; and β-lactalbumin.

In some embodiments, the recombinant fusion protein comprises α-S1 casein, or fragment thereof, and lysozyme. In some embodiments, the recombinant fusion protein comprises α-S2 casein, or fragment thereof, and lysozyme. In some embodiments, the recombinant fusion protein comprises β-casein, or fragment thereof, and lysozyme. In some embodiments, the recombinant fusion protein comprises κ-casein, or fragment thereof, and lysozyme. In some embodiments, the recombinant fusion protein comprises para-κ-casein, or fragment thereof, and lysozyme.

In some embodiments, the recombinant fusion protein comprises α-S1 casein, or fragment thereof; and lactoferrin. In some embodiments, the recombinant fusion protein comprises α-S2 casein, or fragment thereof; and lactoferrin. In some embodiments, the recombinant fusion protein comprises β-casein, or fragment thereof; and lactoferrin. In some embodiments, the recombinant fusion protein comprises κ-casein, or fragment thereof; and lactoferrin. In some embodiments, the recombinant fusion protein comprises para-κ-casein, or fragment thereof; and lactoferrin.

In some embodiments, the recombinant fusion protein comprises α-S1 casein, or fragment thereof; and lactoperoxidase. In some embodiments, the recombinant fusion protein comprises α-S2 casein, or fragment thereof; and lactoperoxidase. In some embodiments, the recombinant fusion protein comprises β-casein, or fragment thereof; and lactoperoxidase. In some embodiments, the recombinant fusion protein comprises κ-casein, or fragment thereof; and lactoperoxidase. In some embodiments, the recombinant fusion protein comprises para-κ-casein, or fragment thereof; and lactoperoxidase.

In some embodiments, the recombinant fusion protein comprises α-S1 casein, or fragment thereof, and an immunoglobulin. In some embodiments, the recombinant fusion protein comprises α-S2 casein, or fragment thereof, and an immunoglobulin. In some embodiments, the recombinant fusion protein comprises β-casein, or fragment thereof, and an immunoglobulin. In some embodiments, the recombinant fusion protein comprises κ-casein, or fragment thereof, and an immunoglobulin. In some embodiments, the recombinant fusion protein comprises para-κ-casein, or fragment thereof, and an immunoglobulin.

In some embodiments, the first protein and the second protein are casein proteins. In some embodiments, the fusion protein comprises κ-casein and para-κ-casein. In some embodiments, the fusion protein comprises κ-casein and β-casein. In some embodiments, the fusion protein comprises κ-casein and α-S1-casein. In some embodiments, the fusion protein comprises κ-casein and α-S2-casein. In some embodiments, the fusion protein comprises para-κ-casein and β-casein. In some embodiments, the fusion protein comprises para-κ-casein and α-S1-casein. In some embodiments, the fusion protein comprises para-κ-casein and α-S2-casein. In some embodiments, the fusion protein comprises β-casein and α-S1-casein. In some embodiments, the fusion protein comprises β-casein and α-S2-casein. In some embodiments, the fusion protein comprises α-S1-casein and α-S2-casein.

In some embodiments, the fusion protein comprises two of the same casein proteins. In some embodiments, the fusion protein comprises a first protein and a second protein, wherein each of the first and second proteins are κ-casein. In some embodiments, the fusion protein comprises a first protein and a second protein, wherein each of the first and second proteins are β-casein. In some embodiments, the fusion protein comprises a first protein and a second protein, wherein each of the first and second proteins are para-κ-casein. In some embodiments, the fusion protein comprises a first protein and a second protein, wherein each of the first and second proteins are α-S1-casein. In some embodiments, the fusion protein comprises a first protein and a second protein wherein each of the first and second proteins are α-S2-casein.

In some embodiments, the fusion protein comprises, form N-terminus to C-terminus, a para-kappa-casein and a beta-lactoglobulin. In some embodiments, the fusion protein comprises, from N-terminus to C-terminus, a beta-lactoglobulin and a para-kappa-casein. In some embodiments, the fusion protein comprises, from N-terminus to C-terminus, an alpha-S1-casein and a beta-lactoglobulin. In some embodiments, the fusion protein comprises, from N-terminus to C-terminus, a beta-lactoglobulin and an alpha-S1-casein. In some embodiments, the fusion protein comprises, from N-terminus to C-terminus, a beta-casein and a beta-lactoglobulin. In some embodiments, the fusion protein comprises from N-terminus to C-terminus, a beta-lactoglobulin and a beta-casein.

### Fusion Proteins Comprising a Milk Protein and a Fusion Partner

In some embodiments, a fusion protein comprises a milk protein and a fusion partner having one or more desirable characteristics. For example, in some embodiments, a fusion protein comprises a first protein and a second protein, wherein the first protein is a milk protein, and the second protein comprises at least one of the following characteristics: (i) a molecular weight of 15 kDa or higher; (ii) at least 30% hydrophobic amino acids; and/or (iii) less than about 2.5 disulfide bonds per 10 kDa molecular weight. In some embodiments, the second protein comprises at least two of the characteristics (i), (ii) and (iii). In some embodiments, the second protein comprises all three of the characteristics (i), (ii) and (iii).

In some embodiments, a fusion protein comprises a milk protein and a fusion partner, wherein the fusion partner has a molecular weight of 15 kDa or higher. In some embodiments, a fusion protein comprises a milk protein and a fusion partner, wherein the fusion partner has at least 30% hydrophobic amino acids. In some embodiments, a fusion protein comprises a milk protein and a fusion partner, wherein the fusion partner has less than about 2.5 disulfide bonds per 10 kDa molecular weight. In some embodiments, a fusion protein comprises a milk protein and a fusion partner, wherein the fusion partner has a molecular weight of 15 kDa or higher, and at least 30% hydrophobic amino acids. In some embodiments, a fusion protein comprises a milk protein and a fusion partner, wherein the fusion partner has at least 30% hydrophobic amino acids, and less than about 2.5 disulfide bonds per 10 kDa molecular weight. In some embodiments, a fusion protein comprises a milk protein and a fusion partner, wherein the fusion partner has a molecular weight of 15 kDa or higher, and less than about 2.5 disulfide bonds per 10 kDa molecular weight. In some embodiments, a fusion protein comprises a milk protein and a fusion partner, wherein the fusion partner has a molecular weight of 15 kDa or higher, at least 30% hydrophobic amino acids, and less than about 2.5 disulfide bonds per 10 kDa molecular weight.

In some embodiments, the fusion protein comprises a protease cleavage site located between the first protein and the second protein. In some embodiments, the protease cleavage site is a chymosin cleavage site. In some embodiments, cleavage of the fusion protein with a protease separates the first protein from the second protein. In some embodiments, after being separated from one another, the first protein and/or the second protein optionally comprise at their N-terminus or C-terminus one or more amino acids that do not occur in the native form of the first protein or the second protein and that are derived from the protease cleavage site.

Fusion Proteins Comprising More than Two Proteins

Fusion proteins may also be created that comprise more than two proteins, such as at least 3, at least 4, at least 5, at least 6, at least 7, at least 8, at least 9, or at least 10, or more proteins. In some embodiments, a fusion protein comprising more than two proteins may comprise at least one milk protein. In some embodiments, a fusion protein comprising more than two proteins may comprise at least one casein protein. In some embodiments, each of the proteins in a fusion protein comprising more than two proteins may be a milk protein. In some embodiments, each of the proteins in a fusion protein comprising more than two proteins may be a casein protein.

In some embodiments, a fusion protein comprising more than two proteins may comprise at least one structured protein and at least one structured protein. In some embodiments, a fusion protein comprising more than two proteins may comprise at least one milk protein (e.g., a casein) and at least one non-milk protein. In some embodiments, a fusion protein comprising more than two proteins may comprise at least one milk protein (e.g., a casein) and at least one plant protein. In some embodiments, a fusion protein comprising more than two proteins may comprise at least one milk protein (e.g., a casein) and at least one animal (e.g., mammalian) protein.

In some embodiments, a fusion protein comprises three proteins, wherein each protein is individually selected from α-S1 casein, α-S2 casein, β-casein, κ-casein, para-κ-casein, β-lactoglobulin, α-lactalbumin, lysozyme, lactoferrin, lactoperoxidase, serum albumin, and an immunoglobulin. In some embodiments, a fusion protein comprises four proteins, wherein each protein is individually selected from α-S1 casein, α-S2 casein, β-casein, κ-casein, para-κ-casein, β-lactoglobulin, α-lactalbumin, lysozyme, lactoferrin, lactoperoxidase, serum albumin, and an immunoglobulin. In some embodiments, a fusion protein comprises five proteins, wherein each protein is individually selected from α-S1 casein, (t-S2 casein, β-casein, κ-casein, para-κ-casein, β-lactoglobulin, α-lactalbumin, lysozyme, lactoferrin, lactoperoxidase, serum albumin, and an immunoglobulin. In some embodiments, a fusion protein comprises six proteins, wherein each protein is individually selected from α-S1 casein, α-S2 casein, β-casein, κ-casein, para-κ-casein, β-lactoglobulin, α-lactalbumin, lysozyme, lactoferrin, lactoperoxidase, serum albumin, and an immunoglobulin. In some embodiments, a fusion protein comprises seven proteins, wherein each protein is individually selected from α-S1 casein, α-S2 casein, 3-casein, κ-casein, para-κ-casein, β-lactoglobulin, α-lactalbumin, lysozyme, lactoferrin, lactoperoxidase, serum albumin, and an immunoglobulin. In some embodiments, a fusion protein comprises eight proteins, wherein each protein is individually selected from α-S1 casein, α-S2 casein, β-casein, κ-casein, para-κ-casein, β-lactoglobulin, α-lactalbumin, lysozyme, lactoferrin, lactoperoxidase, serum albumin, and an immunoglobulin. In some embodiments, a fusion protein comprises nine proteins, wherein each protein is individually selected from α-S1 casein, α-S2 casein, β-casein, κ-casein, para-κ-casein, β-lactoglobulin, α-lactalbumin, lysozyme, lactoferrin, lactoperoxidase, serum albumin, and an immunoglobulin. In some embodiments, a fusion protein comprises ten proteins, wherein each protein is individually selected from α-S1 casein, α-S2 casein, β-casein, κ-casein, para-κ-casein, 3-lactoglobulin, α-lactalbumin, lysozyme, lactoferrin, lactoperoxidase, serum albumin, and an immunoglobulin.

In some embodiments, a fusion protein comprises three proteins, wherein each protein is individually selected from α-S1 casein, α-S2 casein, β-casein, κ-casein, and para-κ-casein. In some embodiments, a fusion protein comprises four proteins, wherein each protein is individually selected from α-S1 casein, α-S2 casein, β-casein, κ-casein, and para-κ-casein. In some embodiments, a fusion protein comprises five proteins, wherein each protein is individually selected from α-S1 casein, α-S2 casein, β-casein, κ-casein, and para-κ-casein. In some embodiments, a fusion protein comprises six proteins, wherein each protein is individually selected from α-S1 casein, α-S2 casein, β-casein, κ-casein, and para-κ-casein. In some embodiments, a fusion protein comprises seven proteins, wherein each protein is individually selected from α-S1 casein, α-S2 casein, β-casein, κ-casein, and para-κ-casein. In some embodiments, a fusion protein comprises eight proteins, wherein each protein is individually selected from α-S1 casein, α-S2 casein, β-casein, κ-casein, and para-κ-casein. In some embodiments, a fusion protein comprises nine proteins, wherein each protein is individually selected from α-S1 casein, α-S2 casein, β-casein, κ-casein, and para-κ-casein. In some embodiments, a fusion protein comprises ten proteins, wherein each protein is individually selected from α-S1 casein, α-S2 casein, β-casein, κ-casein, and para-κ-casein.

In some embodiments, a fusion protein comprises between 3 and 10 proteins, wherein each protein is different. In some embodiments, a fusion protein comprises between 3 and 10 proteins, wherein each protein is the same. In some embodiments, a fusion protein comprises between 3 and 10 proteins, wherein each protein is a milk protein. In some embodiments, a fusion protein comprises between 3 and 10 proteins, wherein each protein is a casein.

In some embodiments, a fusion protein comprises a first, a second, and a third protein, wherein the first protein is beta casein, the second protein is kappa casein, and the third protein is beta-lactoglobulin. See, e.g., SEQ ID NO: 652.

In some embodiments, a fusion protein comprises a first, second, a third, and a fourth protein, wherein the first protein is kappa casein, the second protein is beta casein, the third protein is alpha-S1-casein, and the fourth protein is beta-lactoglobulin. In some embodiments, a fusion protein comprises a first, second, and third protein, wherein the first protein is kappa casein, the second protein is beta casein, the third protein is beta-lactoglobulin. In some embodiments, a fusion protein comprises a first, second, and third protein, wherein the first protein is kappa casein, the second protein is alpha-S1-casein, the third protein is beta-lactoglobulin. In some embodiments, a fusion protein comprises a first, second, and third protein, wherein the first protein is beta-casein, the second protein is alpha-S1-casein, the third protein is beta-lactoglobulin. In some embodiments, a fusion protein comprises a first, second, and third protein, wherein the first protein is kappa-casein, the second protein is beta-casein, the third protein is alpha-S1-casein.

In some embodiments, a fusion protein comprising a first, second, third, and fourth protein, wherein the third protein is kappa-casein. In some embodiments, a fusion protein comprising a first, second, third, and fourth protein, wherein the third protein is kappa-casein, and the fourth protein is beta-lactoglobulin. In some embodiments, the kappa-casein comprises a chymosin cleavage site. In some embodiments, cleavage of the fusion protein with chymosin produces the following polypeptides: (a) a first polypeptide comprising the first protein, the second protein, and para-kappa-casein; (b) a second polypeptide comprising a kappa-casein macropeptide and the fourth protein.

In some embodiments, a fusion protein comprises a first, second, third, and fourth protein, wherein the first protein is beta-casein, the second protein is beta-casein, the third protein is kappa-casein, and the fourth protein is beta-lactoglobulin. See, e.g., SEQ ID NO: 652.

In some embodiments, a fusion protein comprises a first, second, third, fourth, and fifth protein wherein the first protein is beta-casein, the second protein is beta-casein, the third protein is beta-casein, the fourth protein is kappa-casein, and the fifth protein is beta-lactoglobulin. See, e.g., SEQ ID NO: 654.

In some embodiments, a fusion protein comprises a first, second, third, fourth, fifth, and sixth protein wherein the first protein is beta-casein, the second protein is beta-casein, the third protein is beta-casein, the fourth protein is beta-casein, the fifth protein is kappa-casein, and the sixth protein is beta-lactoglobulin. See, e.g., SEQ ID NO: 656.

In some embodiments, a fusion protein comprises a first, second, third, fourth, and fifth protein wherein the first protein is beta-casein, the second protein is beta-casein, the third protein is beta-casein, the fourth protein is beta-casein, and the fifth protein is beta-lactoglobulin. See, e.g., SEQ ID NO: 658 and 662.

In some embodiments, a fusion protein comprises a first, second, third, and fourth protein, wherein the first protein is beta-casein, the second protein is beta-casein, the third protein is beta-casein, and the fourth protein is beta-lactoglobulin. See, e.g., SEQ ID NO: 660.

In some embodiments, a fusion protein comprises a first, second, third, and fourth protein, wherein the first protein is beta-casein, the second protein is beta-casein, the third protein is beta-casein, and the fourth protein is beta-casein. See, e.g., SEQ ID NO: 664.

In some embodiments, a fusion protein comprises β-casein, α-S1 casein, α-S1 casein, and β-casein, in that respective configuration. In some embodiments, a fusion protein is encoded by a nucleic acid sequences that comprises at least about 80%, 85%, 90%, 95%, 97%, 98%, 99%, or up to about 100% identity to SEQ ID NO: 906.

In some embodiments, a fusion protein comprises β-casein, β-casein, κ-casein, and β-lactoglobulin, in that respective configuration. In some embodiments, a fusion protein is encoded by a nucleic acid sequence that comprises at least about 80%, 85%, 90%, 95%, 97%, 98%, 99%, or up to about 100% identity to SEQ ID NO: 908.

In some embodiments, a fusion protein comprises β-casein, β-casein, β-casein, j-casein, in that respective configuration. In some embodiments, a fusion protein is encoded by a nucleic acid sequence that comprises at least about 80%, 85%, 90%, 95%, 97%, 98%, 99%, or up to about 100% identity to SEQ ID NO: 910.

In some embodiments, a fusion protein comprises γ-zein and β-casein, in that respective configuration. In some embodiments, a fusion protein is encoded by a nucleic acid sequence that comprises at least about 80%, 85%, 90%, 95%, 97%, 98%, 99%, or up to about 10000 identity to SEQ ID NO: 912.

Table 5 lists illustrative fusion proteins contemplated by the instant disclosure. The fusion proteins comprise the listed constituent proteins in order from N-terminus to C-terminus. As will be understood by those of skill in the art, in some embodiments, a fusion protein may comprise the constituent proteins in order from C-terminus to N-terminus. In some embodiments, one or more of the fusion proteins may comprise a protease cleavage site, such as a protease cleavage site located between two of the constituent proteins.

TABLE 5
Illustrative Fusion Proteins

| Fusion | | | | | | Sixth |
|---|---|---|---|---|---|---|
| Protein | First | Second | Third | Fourth | Fifth | Pro- |
| No. | Protein | Protein | Protein | Protein | Protein | tein |
| 1 | BC | LG | | | | |
| 2 | BC | BC | LG | | | |
| 3 | BC | BC | KCN | LG | | |
| 4 | BC | BC | BC | KCN | LG | |
| 5 | BC | BC | BC | BC | BC | |
| 6 | BC | aS1 | aS1 | BC | | |
| 7 | BC | aS1 | aS1 | BC | LG | |
| 8 | BC | aS1 | BC | | | |
| 9 | ZN | BC | | | | |
| 10 | ZN27 | BC | | | | |
| 11 | BC | BC | | | | |
| 12 | BC | BC | BC | | | |
| 13 | BC | BC | BC | LG | | |
| 14 | BC | BC | BC | BC | LG | |
| 15 | KCN | BC | BC | BC | | |
| 16 | KCN | BC | BC | | | |
| 17 | KCN | BC | aS1 | LG | | |
| 18 | BC | BC | aS1 | aS1 | BC | BC |
| 19 | paraKCN | paraKCN | paraKCN | BC | BC | |
| 20 | BC | aS1 | KCN | | | |
| 21 | aS1 | LG | | | | |
| 22 | KCN | LG | | | | |
| 23 | paraKCN | LG | | | | |
| 24 | aS1 | aS1 | aS1 | aS1 | | |
| 25 | KCN | KCN | KCN | KCN | | |
| 26 | aS1 | aS1 | aS1 | aS1 | LG | |
| 27 | KCN | KCN | KCN | KCN | LG | |
| 28 | paraKCN | paraKCN | paraKCN | paraKCN | LG | |
| 29 | paraKCN | paraKCN | paraKCN | paraKCN | | |
| 30 | KCN | BC | aS1 | LG | | |
| 31* | KCN | BC | aS1 | | | |
| 32* | KCN | BC | | | | |
| 33* | BC | BC | BC | BC | | |

BC = beta-casein, LG = beta-lactoglobulin, KCN = kappa-casein; paraKCN = para-kappa-casein, aSI = alpha-S1-casein, ZN = truncated zein, ZN27-full-length zein
*indicates that the vector used to express the listed fusion protein also comprises a sequence encoding a Fam kinase, wherein the Fam kinase is expressed under the control of a different promoter.

### Fusion Protein Structure

The fusion proteins described herein may have various structures, to increase expression and/or accumulation in a plant or other host organism or cell. The designation of “first protein”, “second protein”, “third protein”, and/or “fourth protein” is not intended to imply any order.

In some embodiments, the fusion protein may comprise, from N-terminus to C-terminus, the first protein and the second protein. In some embodiments, the fusion protein may comprise, from N-terminus to C-terminus, the second protein and the first protein. In some embodiments, a fusion protein comprises, in order from N-terminus to C-terminus, a first protein and a second protein, wherein the first protein and/or the second protein is a milk protein. In some embodiments, a fusion protein comprises, in order from N-terminus to C-terminus, a second protein and a first protein, wherein the first protein and/or the second protein is a milk protein. For example, in some embodiments, a fusion protein comprises, in order from N-terminus to C-terminus, κ-casein and β-lactoglobulin. In some embodiments, a fusion protein comprises, in order from N-terminus to C-terminus, β-lactoglobulin and κ-casein. In some embodiments, a fusion protein comprises, in order from N-terminus to C-terminus, para-κ-casein and β-lactoglobulin. In some embodiments, a fusion protein comprises, in order from N-terminus to C-terminus, β-lactoglobulin and para-κ-casein. In some embodiments, a fusion protein comprises, in order from N-terminus to C-terminus, β-casein and β-lactoglobulin. In some embodiments, a fusion protein comprises, in order from N-terminus to C-terminus, 0-lactoglobulin and β-casein. In some embodiments, a fusion protein comprises, in order from N-terminus to C-terminus, α-S1 casein and β-lactoglobulin. In some embodiments, a fusion protein comprises, in order from N-terminus to C-terminus, β-lactoglobulin and α-S1 casein.

In some embodiments, a fusion protein comprises, in order from N-terminus to C-terminus, a milk protein and a plant protein. In some embodiments, a fusion protein comprises, in order from N-terminus to C-terminus, a plant protein and a milk protein. In some embodiments, a fusion protein comprises, in order from N-terminus to C-terminus, a casein protein and a plant protein. In some embodiments, a fusion protein comprises, in order from N-terminus to C-terminus, a plant protein and a casein protein.

### Cleavable Fusion Proteins

In some embodiments, it may be desirable to cleave the fusion protein to separate its constituent proteins. For example, it may be desirable to cleave the fusion protein to separate its constituent proteins so that the proteins may individually be used in one or more food compositions.

In some embodiments, a fusion protein comprises a protease cleavage site. For example, in some embodiments, the fusion protein comprises an endoprotease, endopeptidase, and/or endoproteinase cleavage site. In some embodiments, the fusion protein comprises a rennet cleavage site. In some embodiments, the fusion protein comprises a chymosin cleavage site. In some embodiments, the fusion protein comprises a trypsin cleavage site.

The protease cleavage site may be located between the first protein and the second protein. In some embodiments, the protease cleavage site may be located between a milk protein and the non-milk protein. For example, the protease cleavage site may be located between the milk protein and the animal (e.g., mammalian or avian) protein, or between the milk protein and the plant protein, such that cleavage of the protein at the protease cleavage site will separate the two proteins. In some embodiments, the protease cleavage site may be located between a first milk protein and a second milk protein. In some embodiments, the protease cleavage site may be located between a first casein protein and a second casein protein.

In some embodiments, the protease cleavage site may be contained within the sequence of the first protein or the second protein. In some embodiments, the protease cleavage site may be located in either the milk protein or the non-milk protein, for example, the animal (e.g., mammalian or animal) or plant protein. In some embodiments, the protease cleavage site may be added separately, for example, between the two proteins.

In some embodiments, a fusion protein comprises a chymosin cleavage site. In some embodiments, a fusion protein comprises a chymosin cleavage site selected from any one of the sequences shown in Table 6, below. In some embodiments, a fusion protein comprises a chymosin cleavage site that is not shown in Table 6, below. In some embodiments, a fusion protein comprises a chymosin cleavage site having at least 1, at least 2, at least 3 or at least 4 amino acid substitutions relative to any one of the sequences shown in Table 6. In some embodiments, a fusion protein comprises a chymosin cleavage site with a sequence of any one of SEQ ID NO: 665-668, or a sequence having 1, 2, 3, 4, or more amino acid substitutions relative thereto. In the sequences of Table 6, cleavage typically occurs after the underlined residue.

TABLE 6
Chymosin cleavage sites

| Chymosin Cleavage Site | SEQ ID NO: |
|---|---|
| RHPHPHLSFMAIPPKK | 665 |
| HPHPHLSFMAIPPK | 666 |
| RHPHPHLSFM | 667 |
| EDFLQKQQYGISSKFR | 668 |
| RHPHPHLSFMAIPPKK | 669 |
| HHPHPHLSFMAIPPKK | 670 |
| RHPHPRLSFMAIPPKK | 671 |
| RRPRPHLSFMAIPPKK | 672 |
| HQTFQHASFIATPPQK | 673 |
| RRPNLHPSFIAIPPKK | 674 |
| PYAIPNPSFLAMPTNE | 675 |
| PHPIPNPSFLAIPTNE | 676 |
| RHPCPHPSFIAIPPKK | 677 |
| ARRPPHASFIAIPPKK | 678 |
| VGRHSHPFFMAILPNK | 679 |
| RRPRPRPSFIAIPPKK | 680 |
| RHPRPHPSFIAIPPKX | 681 |
| RHPYRRPSFIAIPPKK | 682 |
| RHPHLPASFIVIPPKK | 683 |
| CRRRPHPSFLAIPPXK | 684 |
| HRPNLHPSFIAIPPKK | 685 |
| HRPQLHPSFIAIPPKK | 686 |
| HRPHIHPSFIAIPPKK | 687 |
| HRPHLHPSFIAIPPKK | 688 |
| HRPHLHPSFIAIPAKK | 689 |
| HHPHPCPSFLAIPPKK | 690 |
| HRPHLHPSFTAIPAKK | 691 |
| HHPHPRPSFTAIPPKK | 692 |
| HHPHPRPSFLAIPPKK | 693 |
| HRPHLHPSFIAIPTKK | 694 |
| HHKYLKPSFIVIPPTK | 695 |
| RHPRPHPSFIAIPPKK | 696 |
| YHQAKHPSFMAILSKK | 697 |
| PHTYLKPPFIVIPPKK | 698 |
| HRPKLHPSFIAVPPKK | 699 |
| RRPHPRLSFMAIPPKK | 700 |
| KPAEFFRL | 701 |
| KPAEFKRL | 702 |
| KPAEFERL | 703 |
| KPAEFTRL | 704 |
| KPAEFGRL | 705 |
| KPAEFARL | 706 |
| KPAEFVRL | 707 |
| KPAEFLRL | 708 |
| KPAEFIRL | 709 |
| HPHLSFMAI | 710 |
| HPHLSFEAI | 711 |
| YGIFLRF | 712 |
| YGIFKRF | 713 |
| YGAFLRF | 714 |
| KYSSWYVAL | 715 |
| KYSSWKVAL | 716 |
| KYSSWEVAL | 717 |
| KYSSWLVAL | 718 |
| RPKPQQFFGLM | 719 |
| RPKPQQFKGLM | 720 |
| AFPLEFKREL | 721 |
| AFPLEFKREL | 722 |
| AFPLEFEREL | 723 |
| AFPLEFEREL | 724 |
| AFPLEFIREL | 725 |
| AFPLEFFREL | 726 |
| KIPYILKRQL | 727 |
| KIPYILRRQL | 728 |
| KIPYILERQL | 729 |
| KIPYILSRQL | 730 |
| KIPYILARQL | 731 |
| KIPYILIRQL | 732 |
| KIPYILFRQL | 733 |
| KIPYILFRQL | 734 |
| KIPYILWRQL | 735 |
| EDFLQKQQYGISSKYSGFG | 736 |
| EDFLQKQQYGISSKFM | 737 |
| EDFLQKQQYGISSKFA | 738 |
| EDFLQKQQYGISSKFC | 739 |
| EDFLQKQQYGISSKFF | 740 |
| EDFLQKQQYGISSKFH | 741 |
| EDFLQKQQYGISSKFI | 742 |
| EDFLQKQQYGISSKFK | 743 |
| EDFLQKQQYGISSKFL | 744 |
| EDFLQKQQYGISSKFN | 745 |
| EDFLQKQQYGISSKFR | 746 |
| EDFLQKQQYGISSKFT | 747 |
| EDFLQKQQYGISSKFV | 748 |
| EDFLQKQQYGISSKFW | 749 |
| EDFLQKQQYGISSKYSGFV | 750 |
| EDFLQKQQYGISSKYSGFV | 751 |
| EDFLQKQQYGISSKYSGFM | 752 |
| EDFLQKQQYGISSKYSGFM | 753 |
| EDFLQKQQYGISSKYSGFS | 754 |
| EDFLQKQQYGISSKSSGFV | 755 |
| EDFLQKQQYGISSKSSGFV | 756 |
| EDFLQKQQYGISSKSSGFV | 757 |
| EDFLQKQQYGISSKYV | 758 |
| EDFLQKQQYGISSKFS | 759 |

In some embodiments, a fusion protein comprises a cleavage site recognized by an endoprotease. For example, in some embodiments, a fusion protein comprises a cleavage site selected from any one of the sequences shown in Table 7, below. In some embodiments, a fusion protein comprises a cleavage site having at least 1, at least 2, at least 3 or at least 4 amino acid substitutions relative to any one of the sequences shown in Table 7. In the sequences of Table 7, cleavage typically occurs after the underlined residue.

TABLE 7
Endoprotease Cleavage Sites

| Cleavage Site | SEQ ID NO: | Endoprotease |
|---|---|---|
| DDDDK | 760 | Enterokinase |
| HPHLSFMAI | 761 | Pepsin A |
| HPHLSFEAI | 762 | Pepsin A |
| LVPRG | 763 | Thrombin |
| ELSLSRLRDSA | 764 | Thrombin |
| ELSLSRLR | 765 | Thrombin |
| DNYTRLRK | 766 | Thrombin |
| YTRLRKQM | 767 | Thrombin |
| APSGRVSM | 768 | Thrombin |
| VSMIKNLQ | 769 | Thrombin |
| RIRPKLKW | 770 | Thrombin |
| AMAPRERK | 771 | Thrombin |
| NFFWKTFT | 772 | Thrombin |
| KMYPRGNH | 773 | Thrombin |
| QTYPRTNT | 774 | Thrombin |
| IQGR | 775 | Factor Xa |
| IEGR | 776 | Factor Xa |
| ENLYFQ(G/S) (G/S = G | 777 | TEV protease |
| or S) | | |
| EXXYXQ(G/S) (x = any | 778 | TEV protease |
| amino acid, G/S = G | | |
| or S) | | |
| VDVADX (x = any amino | 779 | Caspase 2 |
| acid) | | |
| RXXR (x = any amino | 780 | Furin |
| acid) | | |
| XX(T/A/S/V)XX (x = any | 781 | Alpha-lytic |
| amino acid) | | protease |

In some embodiments, a fusion protein comprises a cleavage site that is sensitive to cleavage by one or more chemical agents, such as nickel, formic acid, or hydroxylamine. For example, in some embodiments, a fusion protein comprises a chemical cleavage site selected from any one of the sequences shown in Table 8, below. In the sequences of Table 8, cleavage typically occurs after the underlined residue.

TABLE 8
Chemical Cleavage Sites

| Chemical Cleavage Site | SEQ ID NO: | Chemical agent |
|---|---|---|
| GSHHW | 782 | Nickel |
| DP | — | Formic Acid |
| NG | — | Hydroxylamine |

In some embodiments, the fusion protein comprises a protease cleavage site that comprises the amino acids residues F and M (phenylalanine and methionine). Without being bound by any theory, it is believed that one or more enzymes (e.g., chymosin) and cleave between the F and the M. When a protease, such as chymosin, is used to cleave a fusion protein comprising an FM cleavage site, the first protein comprises the F at its C terminus and the second protein comprises a M at its N terminus when liberated from the fusion protein. For example, a protein separated from a fusion protein by cleavage of an FM site may comprise the sequence of any one of SEQ ID NO: 782-791. Thus, in some embodiments, a protein derived from (i.e., separated from) a fusion protein may comprise at least one non-native amino acid. In some embodiments, the non-native amino acid is derived from a protease cleavage site.

In some embodiments, a fusion protein comprises a linker between the first protein and the second protein. In some embodiments, the linker is between the milk protein and the animal (e.g., mammalian or avian) protein, or between the milk protein and the plant protein. In some embodiments, the linker is between a first milk protein and a second milk protein. In some embodiments, the linker is between a first casein protein and a second casein protein. In some embodiments, the linker may comprise a peptide sequence recognizable by an endoprotease. In some embodiments, the linker may comprise a protease cleavage site. In some embodiments, the linker may comprise a self-cleaving peptide, such as a 2A peptide.

In some embodiments, a fusion protein may comprise a signal peptide. The signal peptide may be cleaved from the fusion protein, for example, during processing or transport of the protein within the cell. In some embodiments, the signal peptide is located at the N-terminus of the fusion protein. In some embodiments, the signal peptide is located at the C-terminus of the fusion protein.

In some embodiments, the signal peptide is selected from the group consisting of GmSCB1, StPat21, 2Sss, Sig2, Sig12, Sig8, Sig10, Sig11, and Coixss. In some embodiments, the signal peptide is Sig10 and comprises SEQ ID NO: 15, or a sequence at least 90%, at least 95%, at least 96%, at least 97%, at least 98%, or at least 99% identical thereto. In some embodiments, the signal peptide is Sig2 and comprises SEQ ID NO: 17, or a sequence at least 90%, at least 95%, at least 96%, at least 97%, at least 98%, or at least 99% identical thereto.

In some embodiments, the fusion protein comprises the sequence of SEQ ID NO: 71. In some embodiments, the fusion protein comprises the sequence of SEQ ID NO: 73. In some embodiments, the fusion protein comprises the sequence of SEQ ID NO: 75. In some embodiments, the fusion protein comprises the sequence of SEQ ID NO: 77. In some embodiments, the fusion protein comprises the sequence of SEQ ID NO: 79. In some embodiments, the fusion protein comprises the sequence of SEQ ID NO: 81. In some embodiments, the fusion protein comprises the sequence of SEQ ID NO: 135. In some embodiments, the fusion protein comprises the sequence of SEQ ID NO: 137. In some embodiments, the fusion protein comprises the sequence of SEQ ID NO: 616. In some embodiments, the fusion protein comprises the sequence of SEQ ID NO: 618. In some embodiments, the fusion protein comprises the sequence of SEQ ID NO: 620. In some embodiments, the fusion protein comprises the sequence of SEQ ID NO: 622. In some embodiments, the fusion protein comprises the sequence of SEQ ID NO: 624. In some embodiments, the fusion protein comprises the sequence of SEQ ID NO: 626. In some embodiments, the fusion protein comprises the sequence of SEQ ID NO: 628. In some embodiments, the fusion protein comprises the sequence of SEQ ID NO: 630. In some embodiments, the fusion protein comprises the sequence of SEQ ID NO: 632. In some embodiments, the fusion protein comprises the sequence of SEQ ID NO: 634. In some embodiments, the fusion protein comprises the sequence of SEQ ID NO: 636. In some embodiments, the fusion protein comprises the sequence of SEQ ID NO: 638. In some embodiments, the fusion protein comprises the sequence of SEQ ID NO: 640. In some embodiments, the fusion protein comprises the sequence of SEQ ID NO: 642. In some embodiments, the fusion protein comprises the sequence of SEQ ID NO: 644. In some embodiments, the fusion protein comprises the sequence of SEQ ID NO: 646. In some embodiments, the fusion protein comprises the sequence of SEQ ID NO: 648. In some embodiments, the fusion protein comprises the sequence of SEQ ID NO: 650. In some embodiments, the fusion protein comprises the sequence of SEQ ID NO: 652. In some embodiments, the fusion protein comprises the sequence of SEQ ID NO: 654. In some embodiments, the fusion protein comprises the sequence of SEQ ID NO: 656. In some embodiments, the fusion protein comprises the sequence of SEQ ID NO: 658. In some embodiments, the fusion protein comprises the sequence of SEQ ID NO: 660. In some embodiments, the fusion protein comprises the sequence of SEQ ID NO: 662. In some embodiments, the fusion protein comprises the sequence of SEQ ID NO: 664. In some embodiments, the fusion protein comprises the sequence of SEQ ID NO: 793. In some embodiments, the fusion protein comprises the sequence of SEQ ID NO: 795. In some embodiments, the fusion protein comprises the sequence of SEQ ID NO: 797. In some embodiments, the fusion protein comprises the sequence of SEQ ID NO: 799.

In some embodiments, the fusion protein comprises the sequence of SEQ ID NO: 71, with 1, 2, 3, 4, 5, 6, 7, 8, 9, 10, or more amino acid substitutions. In some embodiments, the fusion protein comprises the sequence of SEQ ID NO: 73, with 1, 2, 3, 4, 5, 6, 7, 8, 9, 10, or more amino acid substitutions. In some embodiments, the fusion protein comprises the sequence of SEQ ID NO: 75, with 1, 2, 3, 4, 5, 6, 7, 8, 9, 10, or more amino acid substitutions. In some embodiments, the fusion protein comprises the sequence of SEQ ID NO: 77, with 1, 2, 3, 4, 5, 6, 7, 8, 9, 10, or more amino acid substitutions. In some embodiments, the fusion protein comprises the sequence of SEQ ID NO: 79, with 1, 2, 3, 4, 5, 6, 7, 8, 9, 10, or more amino acid substitutions. In some embodiments, the fusion protein comprises the sequence of SEQ ID NO: 81, with 1, 2, 3, 4, 5, 6, 7, 8, 9, 10, or more amino acid substitutions. In some embodiments, the fusion protein comprises the sequence of SEQ ID NO: 135, with 1, 2, 3, 4, 5, 6, 7, 8, 9, 10, or more amino acid substitutions. In some embodiments, the fusion protein comprises the sequence of SEQ ID NO: 137, with 1, 2, 3, 4, 5, 6, 7, 8, 9, 10, or more amino acid substitutions. In some embodiments, the fusion protein comprises the sequence of SEQ ID NO: 616, with 1, 2, 3, 4, 5, 6, 7, 8, 9, 10, or more amino acid substitutions. In some embodiments, the fusion protein comprises the sequence of SEQ ID NO: 618, with 1, 2, 3, 4, 5, 6, 7, 8, 9, 10, or more amino acid substitutions. In some embodiments, the fusion protein comprises the sequence of SEQ ID NO: 620, with 1, 2, 3, 4, 5, 6, 7, 8, 9, 10, or more amino acid substitutions. In some embodiments, the fusion protein comprises the sequence of SEQ ID NO: 622, with 1, 2, 3, 4, 5, 6, 7, 8, 9, 10, or more amino acid substitutions. In some embodiments, the fusion protein comprises the sequence of SEQ ID NO: 624, with 1, 2, 3, 4, 5, 6, 7, 8, 9, 10, or more amino acid substitutions. In some embodiments, the fusion protein comprises the sequence of SEQ ID NO: 626, with 1, 2, 3, 4, 5, 6, 7, 8, 9, 10, or more amino acid substitutions. In some embodiments, the fusion protein comprises the sequence of SEQ ID NO: 628, with 1, 2, 3, 4, 5, 6, 7, 8, 9, 10, or more amino acid substitutions. In some embodiments, the fusion protein comprises the sequence of SEQ ID NO: 630, with 1, 2, 3, 4, 5, 6, 7, 8, 9, 10, or more amino acid substitutions. In some embodiments, the fusion protein comprises the sequence of SEQ ID NO: 632, with 1, 2, 3, 4, 5, 6, 7, 8, 9, 10, or more amino acid substitutions. In some embodiments, the fusion protein comprises the sequence of SEQ ID NO: 634, with 1, 2, 3, 4, 5, 6, 7, 8, 9, 10, or more amino acid substitutions. In some embodiments, the fusion protein comprises the sequence of SEQ ID NO: 636, with 1, 2, 3, 4, 5, 6, 7, 8, 9, 10, or more amino acid substitutions. In some embodiments, the fusion protein comprises the sequence of SEQ ID NO: 638, with 1, 2, 3, 4, 5, 6, 7, 8, 9, 10, or more amino acid substitutions. In some embodiments, the fusion protein comprises the sequence of SEQ ID NO: 640, with 1, 2, 3, 4, 5, 6, 7, 8, 9, 10, or more amino acid substitutions. In some embodiments, the fusion protein comprises the sequence of SEQ ID NO: 642, with 1, 2, 3, 4, 5, 6, 7, 8, 9, 10, or more amino acid substitutions. In some embodiments, the fusion protein comprises the sequence of SEQ ID NO: 644, with 1, 2, 3, 4, 5, 6, 7, 8, 9, 10, or more amino acid substitutions. In some embodiments, the fusion protein comprises the sequence of SEQ ID NO: 646, with 1, 2, 3, 4, 5, 6, 7, 8, 9, 10, or more amino acid substitutions. In some embodiments, the fusion protein comprises the sequence of SEQ ID NO: 648, with 1, 2, 3, 4, 5, 6, 7, 8, 9, 10, or more amino acid substitutions. In some embodiments, the fusion protein comprises the sequence of SEQ ID NO: 650, with 1, 2, 3, 4, 5, 6, 7, 8, 9, 10, or more amino acid substitutions. In some embodiments, the fusion protein comprises the sequence of SEQ ID NO: 652, with 1, 2, 3, 4, 5, 6, 7, 8, 9, 10, or more amino acid substitutions. In some embodiments, the fusion protein comprises the sequence of SEQ ID NO: 654, with 1, 2, 3, 4, 5, 6, 7, 8, 9, 10, or more amino acid substitutions. In some embodiments, the fusion protein comprises the sequence of SEQ ID NO: 656, with 1, 2, 3, 4, 5, 6, 7, 8, 9, 10, or more amino acid substitutions. In some embodiments, the fusion protein comprises the sequence of SEQ ID NO: 658, with 1, 2, 3, 4, 5, 6, 7, 8, 9, 10, or more amino acid substitutions. In some embodiments, the fusion protein comprises the sequence of SEQ ID NO: 660, with 1, 2, 3, 4, 5, 6, 7, 8, 9, 10, or more amino acid substitutions. In some embodiments, the fusion protein comprises the sequence of SEQ ID NO: 662, with 1, 2, 3, 4, 5, 6, 7, 8, 9, 10, or more amino acid substitutions. In some embodiments, the fusion protein comprises the sequence of SEQ ID NO: 664, with 1, 2, 3, 4, 5, 6, 7, 8, 9, 10, or more amino acid substitutions. In some embodiments, the fusion protein comprises the sequence of SEQ ID NO: 793, with 1, 2, 3, 4, 5, 6, 7, 8, 9, 10, or more amino acid substitutions. In some embodiments, the fusion protein comprises the sequence of SEQ ID NO: 795, with 1, 2, 3, 4, 5, 6, 7, 8, 9, 10, or more amino acid substitutions. In some embodiments, the fusion protein comprises the sequence of SEQ ID NO: 797, with 1, 2, 3, 4, 5, 6, 7, 8, 9, 10, or more amino acid substitutions. In some embodiments, the fusion protein comprises the sequence of SEQ ID NO: 799, with 1, 2, 3, 4, 5, 6, 7, 8, 9, 10, or more amino acid substitutions.

In some embodiments, the fusion protein comprises the sequence of SEQ ID NO: 71, or a sequence at least 90%, at least 95%, at least 96%, at least 97%, at least 98%, or at least 99% identical thereto. In some embodiments, the fusion protein comprises the sequence of SEQ ID NO: 73, or a sequence at least 90%, at least 95%, at least 96%, at least 97%, at least 98%, or at least 99% identical thereto. In some embodiments, the fusion protein comprises the sequence of SEQ ID NO: 75, or a sequence at least 90%, at least 95%, at least 96%, at least 97%, at least 98%, or at least 99% identical thereto. In some embodiments, the fusion protein comprises the sequence of SEQ ID NO: 77, or a sequence at least 90%, at least 95%, at least 96%, at least 97%, at least 98%, or at least 99% identical thereto. In some embodiments, the fusion protein comprises the sequence of SEQ ID NO: 79, or a sequence at least 90%, at least 95%, at least 96%, at least 97%, at least 98%, or at least 99% identical thereto. In some embodiments, the fusion protein comprises the sequence of SEQ ID NO: 81, or a sequence at least 90%, at least 95%, at least 96%, at least 97%, at least 98%, or at least 99% identical thereto. In some embodiments, the fusion protein comprises the sequence of SEQ ID NO: 135, or a sequence at least 90%, at least 95%, at least 96%, at least 97%, at least 98%, or at least 99% identical thereto. In some embodiments, the fusion protein comprises the sequence of SEQ ID NO: 137, or a sequence at least 90%, at least 95%, at least 96%, at least 97%, at least 98%, or at least 99% identical thereto. In some embodiments, the fusion protein comprises the sequence of SEQ ID NO: 616, or a sequence at least 90%, at least 95%, at least 96%, at least 97%, at least 98%, or at least 99% identical thereto. In some embodiments, the fusion protein comprises the sequence of SEQ ID NO: 618, or a sequence at least 90%, at least 95%, at least 96%, at least 97%, at least 98%, or at least 99% identical thereto. In some embodiments, the fusion protein comprises the sequence of SEQ ID NO: 620, or a sequence at least 90%, at least 95%, at least 96%, at least 97%, at least 98%, or at least 99% identical thereto. In some embodiments, the fusion protein comprises the sequence of SEQ ID NO: 622, or a sequence at least 90%, at least 95%, at least 96%, at least 97%, at least 98%, or at least 99% identical thereto. In some embodiments, the fusion protein comprises the sequence of SEQ ID NO: 624, or a sequence at least 90%, at least 95%, at least 96%, at least 97%, at least 98%, or at least 99% identical thereto. In some embodiments, the fusion protein comprises the sequence of SEQ ID NO: 626, or a sequence at least 90%, at least 95%, at least 96%, at least 97%, at least 98%, or at least 99% identical thereto. In some embodiments, the fusion protein comprises the sequence of SEQ ID NO: 628, or a sequence at least 90%, at least 95%, at least 96%, at least 97%, at least 98%, or at least 99% identical thereto. In some embodiments, the fusion protein comprises the sequence of SEQ ID NO: 630, or a sequence at least 90%, at least 95%, at least 96%, at least 97%, at least 98%, or at least 99% identical thereto. In some embodiments, the fusion protein comprises the sequence of SEQ ID NO: 632, or a sequence at least 90%, at least 95%, at least 96%, at least 97%, at least 98%, or at least 99% identical thereto. In some embodiments, the fusion protein comprises the sequence of SEQ ID NO: 634, or a sequence at least 90%, at least 95%, at least 96%, at least 97%, at least 98%, or at least 99% identical thereto. In some embodiments, the fusion protein comprises the sequence of SEQ ID NO: 636, or a sequence at least 90%, at least 95%, at least 96%, at least 97%, at least 98%, or at least 99% identical thereto. In some embodiments, the fusion protein comprises the sequence of SEQ ID NO: 638, or a sequence at least 90%, at least 95%, at least 96%, at least 97%, at least 98%, or at least 99% identical thereto. In some embodiments, the fusion protein comprises the sequence of SEQ ID NO: 640, or a sequence at least 90%, at least 95%, at least 96%, at least 97%, at least 98%, or at least 99% identical thereto. In some embodiments, the fusion protein comprises the sequence of SEQ ID NO: 642, or a sequence at least 90%, at least 95%, at least 96%, at least 97%, at least 98%, or at least 99% identical thereto. In some embodiments, the fusion protein comprises the sequence of SEQ ID NO: 644, or a sequence at least 90%, at least 95%, at least 96%, at least 97%, at least 98%, or at least 99% identical thereto. In some embodiments, the fusion protein comprises the sequence of SEQ ID NO: 646, or a sequence at least 90%, at least 95%, at least 96%, at least 97%, at least 98%, or at least 99% identical thereto. In some embodiments, the fusion protein comprises the sequence of SEQ ID NO: 648, or a sequence at least 90%, at least 95%, at least 96%, at least 97%, at least 98%, or at least 99% identical thereto. In some embodiments, the fusion protein comprises the sequence of SEQ ID NO: 650, or a sequence at least 90%, at least 95%, at least 96%, at least 97%, at least 98%, or at least 99% identical thereto. In some embodiments, the fusion protein comprises the sequence of SEQ ID NO: 652, or a sequence at least 90%, at least 95%, at least 96%, at least 97%, at least 98%, or at least 99% identical thereto. In some embodiments, the fusion protein comprises the sequence of SEQ ID NO: 654, or a sequence at least 90%, at least 95%, at least 96%, at least 97%, at least 98%, or at least 99% identical thereto. In some embodiments, the fusion protein comprises the sequence of SEQ ID NO: 656, or a sequence at least 90%, at least 95%, at least 96%, at least 97%, at least 98%, or at least 99% identical thereto. In some embodiments, the fusion protein comprises the sequence of SEQ ID NO: 658, or a sequence at least 90%, at least 95%, at least 96%, at least 97%, at least 98%, or at least 99% identical thereto. In some embodiments, the fusion protein comprises the sequence of SEQ ID NO: 660, or a sequence at least 90%, at least 95%, at least 96%, at least 97%, at least 98%, or at least 99% identical thereto. In some embodiments, the fusion protein comprises the sequence of SEQ ID NO: 662, or a sequence at least 90%, at least 95%, at least 96%, at least 97%, at least 98%, or at least 99% identical thereto. In some embodiments, the fusion protein comprises the sequence of SEQ ID NO: 664, or a sequence at least 90%, at least 95%, at least 96%, at least 97%, at least 98%, or at least 99% identical thereto. In some embodiments, the fusion protein comprises the sequence of SEQ ID NO: 793, or a sequence at least 90%, at least 95%, at least 96%, at least 97%, at least 98%, or at least 99% identical thereto. In some embodiments, the fusion protein comprises the sequence of SEQ ID NO: 795, or a sequence at least 90%, at least 95%, at least 96%, at least 97%, at least 98%, or at least 99% identical thereto. In some embodiments, the fusion protein comprises the sequence of SEQ ID NO: 797, or a sequence at least 90%, at least 95%, at least 96%, at least 97%, at least 98%, or at least 99% identical thereto. In some embodiments, the fusion protein comprises the sequence of SEQ ID NO: 799, or a sequence at least 90%, at least 95%, at least 96%, at least 97%, at least 98%, or at least 99% identical thereto.

In some embodiments, the fusion proteins have a molecular weight in the range of about 1 kDa to about 500 kDa, about 1 kDa to about 250 kDa, about 1 to about 100 kDa, about 10 to about 50 kDa, about 1 to about 10 kDa, about 10 to about 200 kDa, about 30 to about 150 kDa, about 30 kDa to about 50 kDa, or about 20 to about 80 kDa.

### Nucleic Acids Encoding Fusion Proteins and Vectors Comprising the Same

Also provided herein are nucleic acids encoding the fusion proteins of the disclosure. In some embodiments, the nucleic acids are DNAs. In some embodiments, the nucleic acids are RNAs.

Also provided herein are examples of expression cassettes for the expression of casein proteins in non-mammalian systems, such as plants and microorganisms, to produce recombinant casein proteins. The expression cassette may comprise, for example, a promoter, a 5′ untranslated region (UTR), a sequence encoding one or more casein proteins, and a terminator. The expression cassette may further comprise a selectable marker and retention signal.

In some embodiments, a nucleic acid comprises a sequence encoding a fusion protein. In some embodiments, a nucleic acid comprises a sequence encoding a fusion protein, which is operably linked to a promoter. In some embodiments, a nucleic acid comprises, in order from 5′ to 3′, a promoter, a 5′ untranslated region (UTR), a sequence encoding a fusion protein, and a terminator.

The promoter may be a plant promoter. A “plant promoter” is a promoter capable of initiating transcription in plant cells. Examples of promoters under developmental control include promoters that preferentially initiate transcription in certain organs, such as leaves, roots, flowers, seeds and tissues such as fibers, xylem vessels, tracheids, or sclerenchyma. Such promoters are referred to as “tissue-preferred.” Promoters which initiate transcription only in certain tissue are referred to as “tissue-specific.” A “cell-type” specific promoter primarily drives expression in certain cell types in one or more organs, for example, vascular cells in leaves, roots, flowers, or seeds. An “inducible” promoter is a promoter which is under environmental control. Examples of environmental conditions that may affect transcription by inducible promoters include anaerobic conditions or the presence of light. Tissue-specific, tissue-preferred, cell-type specific, and inducible promoters constitute the class of “non-constitutive” promoters. A “constitutive” promoter is a promoter which is active under most environmental conditions.

In some embodiments, the promoter is a plant promoter derived from, for example soybean, lima bean, Arabidopsis, tobacco, rice, maize, barley, sorghum, wheat, pea, and/or oat. In some embodiments, the promoter is a constitutive or an inducible promoter. Exemplary constitutive promoters include, but are not limited to, the promoters from plant viruses such as the 35S promoter from CaMV and the promoters from such genes as rice actin; ubiquitin; pEMU; MAS and maize H3 histone. In some embodiments, the constitutive promoter is the ALS promoter, Xbal/Ncol fragment 5′ to the Brassica napus ALS3 structural gene (or a nucleotide sequence similarity to said Xbal/Ncol fragment).

In some embodiments, the promoter is a plant tissue-specific or tissue-preferential promoter. In some embodiments, the promoter is isolated or derived from a soybean gene. Illustrative soybean tissue-specific promoters include AR-Pro1, AR-Pro2, AR-Pro3, AR-Pro4, AR-Pro5, AR-Pro6, AR-Pro7, AR-Pro8, and AR-Pro9.

In some embodiments, the plant is a seed-specific promoter. In some embodiments, the seed-specific promoter is selected from the group consisting of PvPhas, BnNap, AtOlel, GmSeed2, GmSeed3, GmSeed5, GmSeed6, GmSeed7, GmSeed8, GmSeed10, GmSeed11, GmSeed12, pBCON, GmCEP1-L, GmTHIC, GmBg7S1, GmGRD, GmOLEA, GmOLER, Gm2S-1, and GmBBld-II. In some embodiments, the seed-specific promoter is PvPhas and comprises the sequence of SEQ ID NO: 18, or a sequence at least 90%, at least 95%, at least 96%, at least 97%, at least 98%, or at least 99% identical thereto. In some embodiments, the seed-specific promoter is GmSeed2 and comprises the sequence of SEQ ID NO: 19, or a sequence at least 90%, at least 95%, at least 96%, at least 97%, at least 98%, or at least 99% identical thereto. In some embodiments, the promoter is a Cauliflower Mosaic Virus (CaMV) 35S promoter.

In some embodiments, the promoter is a soybean polyubiquitin (Gmubi) promoter, a soybean heat shock protein 90-like (GmHSP90L) promoter, a soybean Ethylene Response Factor (GmERF) promoter. In some embodiments, the promoter is a constitutive soybean promoter derived from GmScreamMl, GmScreamM4, GmScreamM8 genes or GmubiXL genes.

In some embodiments, the 5′ UTR is selected from the group consisting of Arc5′UTR and glnB1UTR. In some embodiments, the 5′ untranslated region is Arc5′UTR and comprises the sequence of SEQ ID NO: 20, or a sequence at least 90%, at least 95%, at least 96%, at least 97%, at least 98%, or at least 99% identical thereto.

In some embodiments, the terminator sequence is isolated or derived from a gene encoding Nopaline synthase, Arc5-1, an Extensin, Rb7 matrix attachment region, a Heat shock protein, Ubiquitin 10, Ubiquitin 3, and M6 matrix attachment region. In some embodiments, the terminator sequence is isolated or derived from a Nopaline synthase gene and comprises the sequence of SEQ ID NO: 22, or a sequence at least 90%, at least 95%, at least 96%, at least 97%, at least 98%, or at least 99% identical thereto.

In some embodiments, the nucleic acid comprises a first terminator sequence and a second terminator sequence (i.e., a dual terminator). In some embodiments, the dual terminator is EU:Rb7. In some embodiments, the dual terminator is AtHSP:AtUbi10. In some embodiments, the dual terminator is EU:StUbi3. In some embodiments, the dual terminator is EU:TM6.

In some embodiments, the dual terminator is EU:Rb7 and comprises the sequence of SEQ ID NO: 138, or a sequence at least 90% at least 95%, at least 96%, at least 97%, at least 98%, or at least 99% identical thereto.

In some embodiments, the dual terminator is AtHSP:AtUbi10 and comprises the sequence of SEQ ID NO: 141, or a sequence at least 90% at least 95%, at least 96%, at least 97%, at least 98%, or at least 99% identical thereto.

In some embodiments, the dual terminator is EU:StUbi3 and comprises the sequence of SEQ ID NO: 144, or a sequence at least 90% at least 95%, at least 96%, at least 97%, at least 98%, or at least 99% identical thereto.

In some embodiments, the dual terminator is EU:TM6 and comprises the sequence of SEQ ID NO: 146, or a sequence at least 90% at least 95%, at least 96%, at least 97%, at least 98%, or at least 99% identical thereto.

In some embodiments, the nucleic acid comprises a 3′ UTR. For example, the 3′ untranslated region may beArc5-1 and comprise SEQ ID NO: 21, or a sequence at least 90%, at least 95%, at least 96%, at least 97%, at least 98%, or at least 99% identical thereto.

In some embodiments the nucleic acid comprises a gene encoding a selectable marker. One illustrative selectable marker gene for plant transformation is the neomycin phosphotransferase II (nptll) gene, isolated from transposon Tn5, which, when placed under the control of plant regulatory signals, confers resistance to kanamycin. Another exemplary marker gene is the hygromycin phosphotransferase gene which confers resistance to the antibiotic hygromycin. In some embodiments, the selectable marker is of bacterial origin and confers resistance to antibiotics such as gentamycin acetyl tranSferase, streptomycin phosphotransferase, and aminoglycoside-3′-adenyl transferase, the bleomycin resistance determinant. In some embodiments, the selectable marker genes confer resistance to herbicides such as glyphosate, glufosinate or bromoxynil. In some embodiments, the selectable marker is mouse dihydrofolate reductase, plant 5-enolpyruvylshikimate-3-phosphate synthase and plant acetolactate synthase. In some embodiments, the selectable marker is acetolactate synthase (e.g., AtCsr1.2).

In some embodiments, a nucleic acid comprises an endoplasmic reticulum retention signal. For example, in some embodiments, a nucleic acid comprises a KDEL sequence (SEQ ID NO: 23). In some embodiments, the nucleic acid may comprise an endoplasmic reticulum retention signal selected from any one of CSEQ ID NO: 23-70.

Shown in Table 9 are exemplary promoters, 5′ UTRs, signal peptides, and terminators that may be used in the nucleic acids of the disclosure.

TABLE 9
Promoters, 5′ UTRs, signal peptides and terminators

| | | | | Illustrative |
|---|---|---|---|---|
| | | | | Accession No. |
| | | | | (Glyma, |
| Type | Name | Description | Native Species | GenBank) |
| Promoter | PvPhas | Phaseolin-1 (aka β- | Common bean | J01263.1 |
| | | phaseolin) | (Phaseolus | |
| | | | vulgaris) | |
| | BnNap | Napin-1 | Rapeseed | J02798.1 |
| | | | (Brassica napus) | |
| | AtOle1 | Oleosin-1 (Ole1) | Arabidopsis | X62353.1, |
| | | | (Arabidopsis | AT4G25140 |
| | | | thaliana) | |
| | GmSeed2 | Gy1 (Glycinin 1) | Soybean | Glyma.03G163500 |
| | | | (Glycine max) | |
| | GmSeed3 | cysteine protease | Soybean | Glyma.08G116300 |
| | | | (Glycine max) | |
| | GmSeed5 | Gy5 (Glycinin 5) | Soybean | Glyma.13G123500 |
| | | | (Glycine max) | |
| | GmSeed6 | Gy4 (Glycinin 4) | Soybean | Glyma.10G037100 |
| | | | (Glycine max) | |
| | GmSeed7 | Kunitz trypsin protease | Soybean | Glyma.01G095000 |
| | | inhibitor | (Glycine max) | |
| | GmSeed8 | Kunitz trypsin protease | Soybean | Glyma.08G341500 |
| | | inhibitor | (Glycine max) | |
| | GmSeed10 | Legume Lectin | Soybean | Glyma.02G012600 |
| | | Domain | (Glycine max) | |
| | GmSeed11 | β-conglycinin a | Soybean | Glyma.20G148400 |
| | | subunit | (Glycine max) | |
| | GmSeed12 | β-conglycinin a′ | Soybean | Glyma.10G246300 |
| | | subunit | (Glycine max) | |
| | pBCON | β-conglycinin β | Soybean | Glyma.20G148200 |
| | | subunit | (Glycine max) | |
| | GmCEP1-L | KDEL-tailed cysteine | Soybean | Glyma06g42780 |
| | | endopeptidase CEP1- | (Glycine max) | |
| | | like | | |
| | GmTHIC | phosphomethylpyrimidine | Soybean | Glyma11g26470 |
| | | synthase | (Glycine max) | |
| | GmBg7S1 | Basic 7S globulin | Soybean | Glyma03g39940 |
| | | precursor | (Glycine max) | |
| | GmGRD | glucose and ribitol | Soybean | Glyma07g38790 |
| | | dehydrogenase-like | (Glycine max) | |
| | GmOLEA | Oleosin isoform A | Soybean | Glyma.19g063400 |
| | | | (Glycine max) | |
| | GmOLEB | Oleosin isoform B | Soybean | Glyma.16g071800 |
| | | | (Glycine max) | |
| | Gm2S-1 | 2S albumin | Soybean | Glyma13g36400 |
| | | | (Glycine max) | |
| | GmBBId-II | Bowman-Birk protease | Soybean | Glyma16g33400 |
| | | inhibitor | (Glycine max) | |
| 5′UTR | Arc5′UTR | arc5-1 gene | Phaseolus | J01263.1 |
| | | | vulgaris | |
| | glnB1UTR | 65 bp of native | Soybean | AF301590.1 |
| | | glutamine synthase | (Glycine max) | |
| Signal peptide | GmSCB1 | Seed coat BURP | Soybean | Glyma07g28940.1 |
| | | domain protein | (Glycine max) | |
| | StPat21 | Patatin | Tomato | CAA27588 |
| | | | (Solanum | |
| | | | lycopersicum) | |
| | 2Sss | 2S albumin | Soybean | Glyma13g36400 |
| | | | (Glycine max) | |
| | Sig2 | Glycinin G1 N- | Soybean | Glyma.03G163500 |
| | | terminal peptide | (Glycine max) | |
| | Sig12 | Beta-conglycinin alpha | Soybean | Glyma.10G246300 |
| | | prime subunit N- | (Glycine max) | |
| | | terminal peptide | | |
| | Sig8 | Kunitz trypsin | Soybean | Glyma.08G341500 |
| | | inhibitor N-terminal | (Glycine max) | |
| | | peptide | | |
| | Sig10 | Lectin N-terminal | Soybean | Glyma.02G012600 |
| | | peptide from Glycine | (Glycine max) | |
| | | max | | |
| | Sig11 | Beta-conglycinin alpha | Soybean | Glyma.20G148400 |
| | | subunit N-terminal | (Glycine max) | |
| | | peptide | | |
| | Coixss | Alpha-coixin N- | Coix lacryma- | |
| | | terminal peptide from | job | |
| | | Coix lacryma-job | | |
| | KDEL | C-terminal amino acids | Phaseolus | |
| | | of sulfhydryl | vulgaris | |
| | | endopeptidase | | |
| Terminator | NOS | Nopaline synthase | Agrobacterium | |
| | | gene termination | tumefaciens | |
| | | sequence | | |
| | ARC | arc5-1 gene | Phaseolus | J01263.1 |
| | | termination sequence | vulgaris | |
| | EU | Extensin termination | Nicotiana | |
| | | sequence | tabacum | |
| | Rb7 | Rb7 matrix attachment | Nicotiana | |
| | | region termination | tabacum | |
| | | sequence | | |
| | HSP or AtHSP | Heat shock termination | Arabidopsis | |
| | | sequence | thaliana | |
| | AtUbi10 | Ubiquitin 10 | Arabidopsis | |
| | | termination sequence | thaliana | |
| | Stubi3 | Ubiquitin 3 | Solanum | |
| | | termination | tuberosum | |
| | TM6 | M6 matrix attachment | Nicotiana | |
| | | region termination | tabacum | |
| | | sequence | | |
| Dual | EU:Rb7 | Extensin termination | Nicotiana | |
| terminators | | sequence:Rb7 matrix | tabacum | |
| | | attachment region | | |
| | | termination sequence | | |
| | AtHSP:AtUbi10 | Heat shock termination | Arabidopsis | |
| | | sequence:Ubiquitin 10 | thaliana | |
| | | termination sequence | | |
| | EU:StUbi3 | Rb7 matrix attachment | Nicotiana | |
| | | region termination | tabacum, | |
| | | sequence:Ubiquitin 3 | Solanum | |
| | | termination | tuberosum | |
| | EU:TM6 | Rb7 matrix attachment | Nicotiana | |
| | | region termination | tabacum | |
| | | sequence:M6 matrix | | |
| | | attachment region | | |
| | | termination sequence | | |

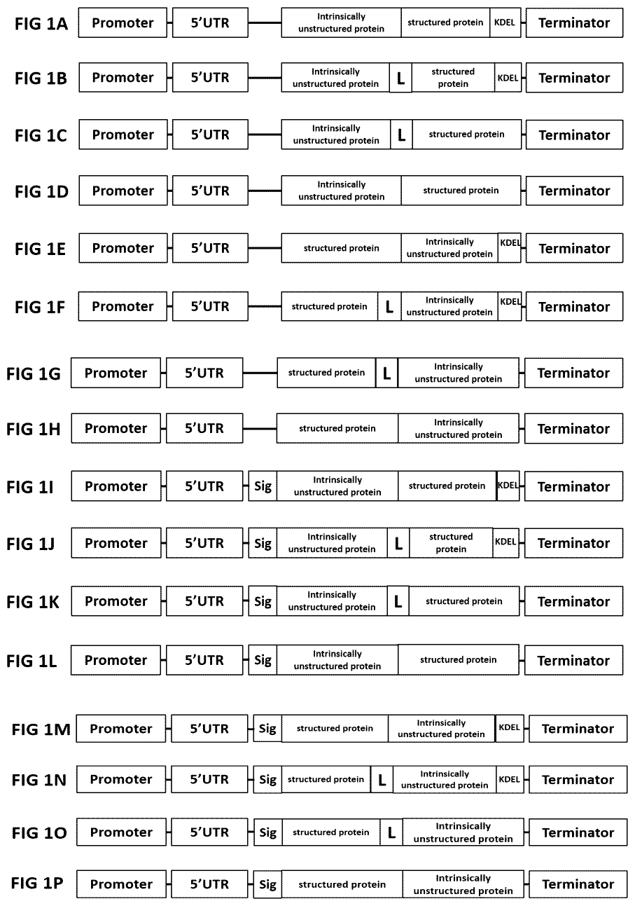

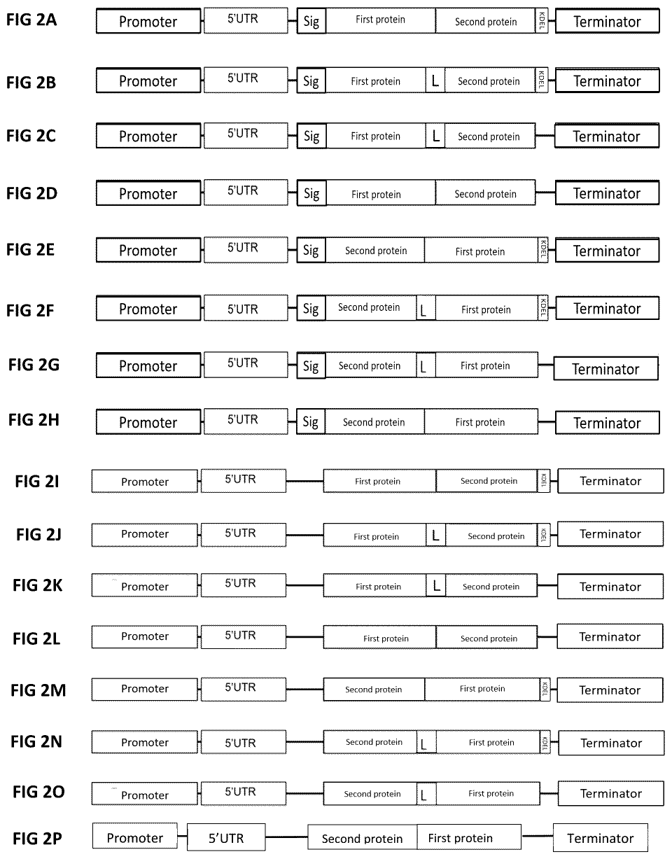

Illustrative nucleic acids of the disclosure are provided in FIG. 1A-FIG. 1P and FIG. 2A-FIG. 2P. In some embodiments a nucleic acid comprises, from 5′ to 3′, a promoter, a 5′UTR, a sequence encoding an unstructured milk protein, a sequence encoding a structured mammalian protein, an endoplasmic reticulum retention signal, and a terminator (See, e.g., FIG. 1A). In some embodiments a nucleic acid comprises, from 5′ to 3′, a promoter, a 5′UTR, a sequence encoding an unstructured milk protein, a sequence encoding a linker, a sequence encoding a structured mammalian protein, an endoplasmic reticulum retention signal, and a terminator (See, e.g., FIG. 1B). In some embodiments a nucleic acid comprises, from 5′ to 3′, a promoter, a 5′UTR, a sequence encoding an unstructured milk protein, a sequence encoding a linker, a sequence encoding a structured mammalian protein, and a terminator (See, e.g., FIG. 1C). In some embodiments a nucleic acid comprises, from 5′ to 3′, a promoter, a 5′UTR, a sequence encoding an unstructured milk protein, a sequence encoding a structured mammalian protein, and a terminator (See, e.g., FIG. 1D). In some embodiments a nucleic acid comprises, from 5′ to 3′, a promoter, a 5′UTR, a sequence encoding a structured mammalian protein, a sequence encoding an unstructured milk protein, an endoplasmic reticulum retention signal, and a terminator (See, e.g., FIG. 1E). In some embodiments a nucleic acid comprises, from 5′ to 3′, a promoter, a 5′UTR, a sequence encoding a structured mammalian protein, a sequence encoding a linker, a sequence encoding an unstructured milk protein, an endoplasmic reticulum retention signal, and a terminator (See, e.g., FIG. 1F). In some embodiments a nucleic acid comprises, from 5′ to 3′, a promoter, a 5′UTR, a sequence encoding a structured mammalian protein, a sequence encoding a linker, a sequence encoding an unstructured milk protein, and a terminator (See, e.g., FIG. 1G). In some embodiments a nucleic acid comprises, from 5′ to 3′, a promoter, a 5′UTR, a sequence encoding a structured mammalian protein, a sequence encoding an unstructured milk protein, and a terminator (See, e.g., FIG. 1H). In some embodiments a nucleic acid comprises, from 5′ to 3′, a promoter, a 5′UTR, a sequence encoding a signal peptide, a sequence encoding an unstructured milk protein, a sequence encoding a structured mammalian protein, an endoplasmic reticulum retention signal, and a terminator (See, e.g., FIG. 1I). In some embodiments a nucleic acid comprises, from 5′ to 3′, a promoter, a 5′UTR, a sequence encoding a signal peptide, a sequence encoding an unstructured milk protein, a sequence encoding a linker, a sequence encoding a structured mammalian protein, an endoplasmic reticulum retention signal, and a terminator (See, e.g., FIG. 1J). In some embodiments a nucleic acid comprises, from 5′ to 3′, a promoter, a 5′UTR, a sequence encoding a signal peptide, a sequence encoding an unstructured milk protein, a sequence encoding a linker, a sequence encoding a structured mammalian protein, and a terminator (See, e.g., FIG. 1K). In some embodiments a nucleic acid comprises, from 5′ to 3′, a promoter, a 5′UTR, a sequence encoding a signal peptide, a sequence encoding an unstructured milk protein, a sequence encoding a structured mammalian protein, and a terminator (See, e.g., FIG. 1L). In some embodiments a nucleic acid comprises, from 5′ to 3′, a promoter, a 5′UTR, a sequence encoding a signal peptide, a sequence encoding a structured mammalian protein, a sequence encoding an unstructured milk protein, an endoplasmic reticulum retention signal, and a terminator (See, e.g., FIG. 1M). In some embodiments a nucleic acid comprises, from 5′ to 3′, a promoter, a 5′UTR, a sequence encoding a signal peptide, a sequence encoding a structured mammalian protein, a sequence encoding a linker, a sequence encoding an unstructured milk protein, an endoplasmic reticulum retention signal, and a terminator (See, e.g., FIG. 1N). In some embodiments a nucleic acid comprises, from 5′ to 3′, a promoter, a 5′UTR, a sequence encoding a signal peptide, a sequence encoding a structured mammalian protein, a sequence encoding a linker, a sequence encoding an unstructured milk protein, and a terminator (See, e.g., FIG. 1O). In some embodiments a nucleic acid comprises, from 5′ to 3′, a promoter, a 5′UTR, a sequence encoding a signal peptide, a sequence encoding a structured mammalian protein, a sequence encoding an unstructured milk protein, and a terminator (See, e.g., FIG. 1P).

In some embodiments a nucleic acid comprises, from 5′ to 3′, a promoter, a 5′UTR, a sequence encoding a signal peptide, a sequence encoding first protein, a sequence encoding a second protein, an endoplasmic reticulum retention signal, and a terminator (See, e.g., FIG. 2A). In some embodiments a nucleic acid comprises, from 5′ to 3′, a promoter, a 5′UTR, a sequence encoding a signal peptide, a sequence encoding first protein, a sequence encoding a linker, a sequence encoding a second protein, an endoplasmic reticulum retention signal, and a terminator (See, e.g., FIG. 2B). In some embodiments a nucleic acid comprises, from 5′ to 3′, a promoter, a 5′UTR, a sequence encoding a signal peptide, a sequence encoding a first protein, a sequence encoding a linker, a sequence encoding a second protein, and a terminator (See, e.g., FIG. 2C). In some embodiments a nucleic acid comprises, from 5′ to 3′, a promoter, a 5′UTR, a sequence encoding a signal peptide, a sequence encoding a first protein, a sequence encoding a second protein, and a terminator (See, e.g., FIG. 2D). In some embodiments a nucleic acid comprises, from 5′ to 3′, a promoter, a 5′UTR, a sequence encoding a signal peptide, a sequence encoding a second protein, a sequence encoding a first protein, an endoplasmic reticulum retention signal, and a terminator (See, e.g., FIG. 2E). In some embodiments a nucleic acid comprises, from 5′ to 3′, a promoter, a 5′UTR, a sequence encoding a signal peptide, a sequence encoding a second protein, a sequence encoding a linker, a sequence encoding a first protein, an endoplasmic reticulum retention signal, and a terminator (See, e.g., FIG. 2F). In some embodiments a nucleic acid comprises, from 5′ to 3′, a promoter, a 5′UTR, a sequence encoding a signal peptide, a sequence encoding a second protein, a sequence encoding a linker, a sequence encoding a first protein, and a terminator (See, e.g., FIG. 2G). In some embodiments a nucleic acid comprises, from 5′ to 3′, a promoter, a 5′UTR, a sequence encoding a signal peptide, a sequence encoding a second protein, a sequence encoding first protein, and a terminator (See, e.g., FIG. 2H).

In some embodiments a nucleic acid comprises, from 5′ to 3′, a promoter, a 5′UTR, a sequence encoding a first protein, a sequence encoding a second protein, an endoplasmic reticulum retention signal, and a terminator (See, e.g., FIG. 2I). In some embodiments a nucleic acid comprises, from 5′ to 3′, a promoter, a 5′UTR, a sequence encoding a first protein, a sequence encoding a linker, a sequence encoding a second protein, an endoplasmic reticulum retention signal, and a terminator (See, e.g., FIG. 2J). In some embodiments a nucleic acid comprises, from 5′ to 3′, a promoter, a 5′UTR, a sequence encoding a first protein, a sequence encoding a linker, a sequence encoding a second protein, and a terminator (See, e.g., FIG. 2K). In some embodiments a nucleic acid comprises, from 5′ to 3′, a promoter, a 5′UTR, a sequence encoding a first protein, a sequence encoding a second protein, and a terminator (See, e.g., FIG. 2L). In some embodiments a nucleic acid comprises, from 5′ to 3′, a promoter, a 5′UTR, a sequence encoding a second protein, a sequence encoding a first protein, an endoplasmic reticulum retention signal, and a terminator (See, e.g., FIG. 2M). In some embodiments a nucleic acid comprises, from 5′ to 3′, a promoter, a 5′UTR, a sequence encoding a second protein, a sequence encoding a linker, a sequence encoding a first protein, an endoplasmic reticulum retention signal, and a terminator (See, e.g., FIG. 2N). In some embodiments a nucleic acid comprises, from 5′ to 3′, a promoter, a 5′UTR, a sequence encoding a second protein, a sequence encoding a linker, a sequence encoding a first protein, and a terminator (See, e.g., FIG. 2O). In some embodiments a nucleic acid comprises, from 5′ to 3′, a promoter, a 5′UTR, a sequence encoding a second protein, a sequence encoding a first protein, and a terminator (See, e.g., FIG. 2P).

In some embodiments, the nucleic acid comprises an expression cassette comprising a OKC1-T:OLG1 (Optimized Kappa Casein version 1:beta-lactoglobulin version 1) fusion driven by PvPhas promoter fused with arc5′UTR:sig10, followed by the ER retention signal (KDEL) and the 3′UTR of the arc5-1 gene, “arc-terminator” (See, e.g., FIG. 4). In some embodiments, the nucleic acid comprises SEQ ID NO: 72.

In some embodiments, the nucleic acid comprises an expression cassette comprising a OBC-T2:FM:OLG1 (Optimized Beta Casein Truncated version 2:Chymosin cleavage site:beta-lactoglobulin version 1) fusion driven by PvPhas promoter fused with arc5′UTR:sig10, followed by the 3′UTR of the arc5-1 gene, “arc-terminator” (See, e.g., FIG. 5). In some embodiments, the nucleic acid comprises SEQ ID NO: 74. The Beta Casein is “truncated” in that the bovine secretion signal is removed and replaced with a plant targeting signal.

In some embodiments, the nucleic acid comprises an expression cassette comprising a OaS1-T:FM:OLG1 (Optimized Alpha S1 Casein Truncated version 1:Chymosin cleavage site:beta-lactoglobulin version 1) fusion driven by PvPhas promoter fused with arc5′UTR:sig10, followed by the 3′UTR of the arc5-1 gene, “arc-terminator” (See, e.g., FIG. 6). In some embodiments, the nucleic acid comprises SEQ ID NO: 76. The Alpha S1 is “truncated” in that the bovine secretion signal is removed and replaced with a plant targeting signal.

In some embodiments, the nucleic acid comprises an expression cassette comprising a para-OKC1-T:FM:OLG1:KDEL (Optimized paraKappa Casein version 1:Chymosin cleavage site:beta-lactoglobulin version 1) fusion driven by PvPhas promoter fused with arc5′UTR:sig 10, followed by the ER retention signal (KDEL) and the 3′UTR of the arc5-1 gene, “arc-terminator” (See, e.g., FIG. 7). In some embodiments, the nucleic acid comprises SEQ ID NO: 78.

In some embodiments, the nucleic acid comprises an expression cassette comprising a para-OKC1-T:FM:OLG1 (Optimized paraKappa Casein version 1:Chymosin cleavage site:beta-lactoglobulin version 1) fusion driven by PvPhas promoter fused with arc5′UTR:sig 10, followed by the 3′UTR of the arc5-1 gene, “arc-terminator” (See, e.g., FIG. 8). In some embodiments, the nucleic acid comprises SEQ ID NO: 80.

In some embodiments, the nucleic acid comprises an expression cassette comprising a OKC1-T-OLG1 (Optimized Kappa Casein version 1:beta-lactoglobulin version 1) fusion that is driven by the promoter and signal peptide of glycinin 1 (GmSeed2:sig2) followed by the ER retention signal (KDEL) and the nopaline synthase gene termination sequence (nos term) (See, e.g., FIG. 9). In some embodiments, the nucleic acid comprises SEQ ID NO: 82.

In some embodiments, a nucleic acid encoding a fusion protein comprises the sequence of any one of SEQ ID NO: 72, 74, 76, 78, 80, 82, 134, or 136. In some embodiments, a nucleic acid encoding a fusion protein comprises the sequence of any one of SEQ ID NO: 615, 617, 619, 621, 623, 625, 627, 629, 631, 633, 635, 637, 639, 641, 643, 645, 647, 649, 651, 653, 655, 657, 659, 661, 663, 792, 794, 796, or 798.

In some embodiments, the nucleic acids are codon optimized for expression in a host cell. Codon optimization is a process used to improve gene expression and increase the translational efficiency of a gene of interest by accommodating codon bias of the host organism (i.e., the organism in which the gene is expressed). Codon-optimized mRNA sequences that are produced using different programs or approaches can vary because different codon optimization strategies differ in how they quantify codon usage and implement codon changes. Some approaches use the most optimal (frequently used) codon for all instances of an amino acid, or a variation of this approach. Other approaches adjust codon usage so that it is proportional to the natural distribution of the host organism. These approaches include codon harmonization, which endeavors to identify and maintain regions of slow translation thought to be important for protein folding. Alternative approaches involve using codons thought to correspond to abundant tRNAs, using codons according to their cognate tRNA concentrations, selectively replacing rare codons, or avoiding occurrences of codon-pairs that are known to translate slowly. In addition to approaches that vary in the extent to which codon usage is considered as a parameter, there are hypothesis-free approaches that do not consider this parameter. Algorithms for performing codon optimization are known to those of skill in the art and are widely available on the Internet.

In some embodiments the nucleic acids are codon optimized for expression in a plant species. The plant species may be, for example, a monocot or a dicot. In some embodiments, the plant species is a dicot species selected from soybean, lima bean, Arabidopsis, tobacco, rice, maize, barley, sorghum, wheat and/or oat. In some embodiments, the plant species is soybean.

In some embodiments, the nucleic acids are codon optimized for expression in a eukaryotic microorganism. The species may be, for example, Saccharomyces spp., Kluyveromyces spp., Pichia spp., Aspergillus spp., Tetrahymena spp., Yarrowla spp., Hansenula spp., Blastobotrys spp., Candida spp., Zygosaccharomyces spp., Debrayomyces spp., Fusarium spp., and Trichoderma spp.

In some embodiments, the nucleic acids are codon optimized for expression in a bacterial cell. The bacterial species may be, for example, Escherichia coli, Caulobacter crescentus, Rodhobacter sphaeroides, Pseudoalteromonas haloplanktis, Shewanella sp., Pseudomonas putida, P. aeruginosa, P. fluorescens, Halomonas elongate, Chromohalobacter salexigens, Streptomyces lividans, S. griseus, Nocardia lactamdurans, Mycobacterium smegmatis, Corynebacterium glutamicum, C. ammoniagenes, Brevibacterium lactofermentum, Bacillus subtilis, B. brevis, B. megaterium, B. licheniformis, B. amyloliquefaciens, Lactococcus lactis, L. plantarum, L. casei, L. reuteri, L. gasseri.

In some embodiments, a nucleic acid may encode more than one fusion protein. For example, in some embodiments, a nucleic acid may encode two, three, four, five, six, seven, eight, nine, or ten fusion proteins. Expression of each fusion protein in the nucleic acid may be driven by a separate promoter. For example, in some embodiments, a nucleic acid comprises a first promoter configured to drive expression of a sequence encoding a first fusion protein, and a second promoter configured to drive expression of a sequence encoding a second fusion protein. In some embodiments, a nucleic acid comprises a first promoter operably linked to a sequence encoding a first fusion protein, and a second promoter operably linked to a sequence encoding a second fusion protein.

The nucleic acids of the disclosure may be contained within a vector. The vector may be, for example, a viral vector or a non-viral vector. In some embodiments, the non-viral vector is a plasmid, such as an Agrobacterium Ti plasmid. In some embodiments, the non-viral vector is a lipid nanoparticle.

In some embodiments, the vector comprises a nucleic acid encoding multiple fusion proteins. For example, in some embodiments, a vector comprises a nucleic acid comprising a sequence encoding a first fusion protein and a sequence encoding a second fusion protein. A first promoter may drive expression of the first fusion protein, and a second promoter may drive expression of the second fusion protein. In some embodiments, the first promoter and the second promoter are the same. In some embodiments, the first promoter and the second promoter are different. In some embodiments, a vector comprises a nucleic acid comprising a sequence encoding a first fusion protein, a sequence encoding a second fusion protein, and a sequence encoding a third fusion protein. A first promoter may drive expression of the first fusion protein, a second promoter may drive expression of the second fusion protein, and a third promoter may drive expression of the third fusion protein. In some embodiments, each of the first, second, and third promoter are different. In some embodiments, at least two of the first, second, and third promoter are different. In some embodiments, the first, second, and third promoter are the same.

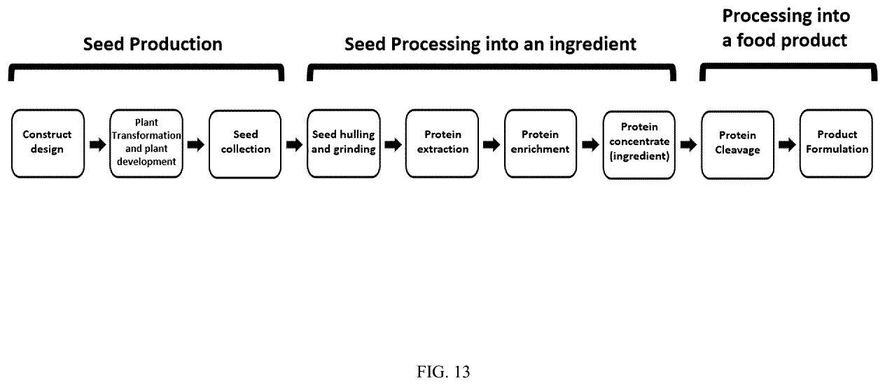

In some embodiments, a vector comprises a nucleic acid encoding a recombinant fusion protein, wherein the recombinant fusion protein comprises: (i) an unstructured milk protein, and (ii) a structured animal (e.g., mammalian or avian) protein. In some embodiments, the vector is an Agrobacterium Ti plasmid. In some embodiments, a vector comprises a nucleic acid encoding a recombinant fusion protein, wherein the recombinant fusion protein comprises: (1) a milk protein, and (2) a second protein. In some embodiments, the second protein is also a milk protein. In some embodiments, the second protein is beta-lactoglobulin. In some embodiments, the second protein is a mammalian or avian protein. In some embodiments, the vector is an Agrobacterium Ti plasmid. In some embodiments, the vector is a vector for use with an Agrobacterium binary vector transformation system. In some embodiments, the fusion protein is cleaved to liberate the milk protein and the second protein before either one is used to prepare a composition as described herein (See, e.g., FIG. 13). The fusion protein may be cleaved, for example, with one or more proteases.

In some embodiments, a method for expressing a casein protein (including fusion proteins comprising a casein protein) in a plant comprises contacting the plant with a vector of the disclosure. In some embodiments, a method for expression of a casein protein in a plant comprises contacting the plant with an Agrobacterium cell comprising a vector of the disclosure. In some embodiments, the method comprises maintaining the plant or part thereof under conditions in which the fusion protein is expressed.

In some embodiments, a method for expressing a fusion protein in a plant comprises contacting the plant with a vector of the disclosure. In some embodiments, the method comprises maintaining the plant or part thereof under conditions in which the fusion protein is expressed.

### Plants Expressing Fusion Proteins

Also provided herein are transgenic plants expressing one or more fusion proteins of the disclosure. In some embodiments, the transgenic plants stably express the fusion protein. In some embodiments, the transgenic plants transiently express the fusion protein. In some embodiments, the transgenic plants stably express the fusion protein in the plant in an amount of at least 1% per the total protein weight of the soluble protein extractable from the plant. For example, the transgenic plants may stably express the fusion protein in an amount of at least 1%, at least 1.5%, at least 2%, at least 2.5%, at least 3%, at least 3.5%, at least 4%, at least 4.5%, at least 5%, at least 5.5%, at least 6%, at least 6.5%, at least 7%, at least 7.5%, at least 8%, at least 8.5%, at least 9%, at least 9.5%, at least 10%, at least 10.5%, at least 11%, at least 11.5%, at least 12%, at least 12.5%, at least 13%, at least 13.5%, at least 14%, at least 14.5%, at least 15%, at least 15.5%, at least 16%, at least 16.5%, at least 17%, at least 17.5%, at least 18%, at least 18.5%, at least 19%, at least 19.5%, at least 20%, or more of total protein weight of soluble protein extractable from the plant (or specific plant part, such as a bean). In some embodiments, the transgenic plants stably express a milk protein (e.g., within a fusion protein), wherein the milk protein expresses in an amount of at least 1%, at least 1.5%, at least 2%, at least 2.5%, at least 3%, at least 3.5%, at least 4%, at least 4.5%, at least 5%, at least 5.5%, at least 6%, at least 6.5%, at least 7%, at least 7.5%, at least 8%, at least 8.5%, at least 9%, at least 9.5%, at least 10%, at least 10.5%, at least 11%, at least 11.5%, at least 12%, at least 12.5%, at least 13%, at least 13.5%, at least 14%, at least 14.5%, at least 15%, at least 15.5%, at least 16%, at least 16.5%, at least 17%, at least 17.5%, at least 18%, at least 18.5%, at least 19%, at least 19.5%, at least 20%, or more of total protein weight of soluble protein extractable from the plant (or specific plant part, such as a bean).

In some embodiments, the transgenic plants stably express the fusion protein in an amount of less than about 1% of the total protein weight of soluble protein extractable from the plant. In some embodiments, the transgenic plants stably express the fusion protein in the range of about 1% to about 2%, about 3% to about 4%, about 4% to about 5%, about 5% to about 6%, about 6% to about 7%, about 7% to about 8%, about 8% to about 9%, about 9% to about 10%, about 10% to about 11%, about 11% to about 12%, about 12% to about 13%, about 13% to about 14%, about 14% to about 15%, about 15% to about 16%, about 16% to about 17%, about 17%, to about 18%, about 18% to about 19%, about 19% to about 20%, or more than about 20% of the total protein weight of soluble protein extractable from the plant (or specific plant part, such as a bean).

In some embodiments, the transgenic plants stably express a milk protein (e.g., within a fusion protein) in the range of about 1% to about 2%, about 3% to about 4%, about 4% to about 5%, about 5% to about 6%, about 6% to about 7%, about 7% to about 8%, about 8% to about 9%, about 9% to about 10%, about 10% to about 11%, about 11% to about 12%, about 12% to about 13%, about 13% to about 14%, about 14% to about 15%, about 15% to about 16%, about 16% to about 17%, about 17%, to about 18%, about 18% to about 19%, about 19% to about 20%, or more than about 20% of the total protein weight of soluble protein extractable from the plant (or specific plant part, such as a bean).

In some embodiments, the transgenic plant stably expresses the fusion protein (or a milk protein within the fusion) in an amount in the range of about 0.5% to about 3%, about 1% to about 4%, about 1% to about 5%, about 2% to about 5%, about 1% to about 10%, about 2% to about 10%, about 3% to about 10%, about 5 to about 12%, about 4% to about 10%, or about 5% to about 10%, about 4% to about 8%, about 5% to about 15%, about 5% to about 18%, about 10% to about 20%, or about 1% to about 20% of the total protein weight of soluble protein extractable from the plant (or specific plant part, such as a bean).

In some embodiments, the fusion protein (or a milk protein within the fusion) is expressed at a level at least 2-fold higher than a milk protein expressed individually (i.e., expressed alone, not as part of a fusion protein) in a plant. For example, in some embodiments, the fusion protein is expressed at a level at least 2-fold, at least 2.5-fold, at least 3-fold, at least 3.5-fold, at least 4-fold, at least 4.5-fold, at least 5-fold, at least 5.5-fold, at least 6-fold, at least 7-fold, at least 7.5-fold, at least 8-fold, at least 8.5-fold, at least 9-fold, at least 9.5-fold, at least 10-fold, at least 25-fold, at least 50-fold, or at least 100-fold higher than a milk protein expressed individually in a plant.

In some embodiments, the fusion protein allows for accumulation of a casein protein in the plant at least 2-fold higher than a casein protein expressed individually (i.e., expressed alone, not as a part of a fusion protein) in a plant. For example, in some embodiments, the casein protein expressed in a fusion protein accumulates in the plant at least 2-fold, at least 2.5-fold, at least 3-fold, at least 3.5-fold, at least 4-fold, at least 4.5-fold, at least 5-fold, at least 5.5-fold, at least 6-fold, at least 7-fold, at least 7.5-fold, at least 8-fold, at least 8.5-fold, at least 9-fold, at least 9.5-fold, at least 10-fold, at least 25-fold, at least 50-fold, or at least 100-fold higher than a casein protein expressed individually.

In some embodiments, the fusion protein is stably expressed in the plant in an amount of 1% or higher per the total protein weight of the soluble protein extractable from the plant (or specific plant part, such as a bean). In some embodiments, the fusion protein is stably expressed in the plant in an amount of 2% or higher per the total protein weight of the soluble protein extractable from the plant (or specific plant part, such as a bean). In some embodiments, the fusion protein is stably expressed in the plant in an amount of 3% or higher per the total protein weight of the soluble protein extractable from the plant (or specific plant part, such as a bean). In some embodiments, the fusion protein is stably expressed in the plant in an amount of 4% or higher per the total protein weight of the soluble protein extractable from the plant (or specific plant part, such as a bean). In some embodiments, the fusion protein is stably expressed in the plant in an amount of 5% or higher per the total protein weight of the soluble protein extractable from the plant (or specific plant part, such as a bean). In some embodiments, the fusion protein is stably expressed in the plant in an amount of 6% or higher per the total protein weight of the soluble protein extractable from the plant (or specific plant part, such as a bean). In some embodiments, the fusion protein is stably expressed in the plant in an amount of 7% or higher per the total protein weight of the soluble protein extractable from the plant (or specific plant part, such as a bean). In some embodiments, the fusion protein is stably expressed in the plant in an amount of 8% or higher per the total protein weight of the soluble protein extractable from the plant (or specific plant part, such as a bean). In some embodiments, the fusion protein is stably expressed in the plant in an amount of 9% or higher per the total protein weight of the soluble protein extractable from the plant (or specific plant part, such as a bean). In some embodiments, the fusion protein is stably expressed in the plant in an amount of 10% or higher per the total protein weight of the soluble protein extractable from the plant (or specific plant part, such as a bean). In some embodiments, the fusion protein is stably expressed in the plant in an amount of 110% or higher per the total protein weight of the soluble protein extractable from the plant (or specific plant part, such as a bean). In some embodiments, the fusion protein is stably expressed in the plant in an amount of 12% or higher per the total protein weight of the soluble protein extractable from the plant (or specific plant part, such as a bean). In some embodiments, the fusion protein is stably expressed in the plant in an amount of 13% or higher per the total protein weight of the soluble protein extractable from the plant (or specific plant part, such as a bean). In some embodiments, the fusion protein is stably expressed in the plant in an amount of 14% or higher per the total protein weight of the soluble protein extractable from the plant (or specific plant part, such as a bean). In some embodiments, the fusion protein is stably expressed in the plant in an amount of 15% or higher per the total protein weight of the soluble protein extractable from the plant (or specific plant part, such as a bean). In some embodiments, the fusion protein is stably expressed in the plant in an amount of 16% or higher per the total protein weight of the soluble protein extractable from the plant (or specific plant part, such as a bean). In some embodiments, the fusion protein is stably expressed in the plant in an amount of 17% or higher per the total protein weight of the soluble protein extractable from the plant (or specific plant part, such as a bean). In some embodiments, the fusion protein is stably expressed in the plant in an amount of 18% or higher per the total protein weight of the soluble protein extractable from the plant (or specific plant part, such as a bean). In some embodiments, the fusion protein is stably expressed in the plant in an amount of 19% or higher per the total protein weight of the soluble protein extractable from the plant. In some embodiments, the fusion protein is stably expressed in the plant in an amount of 20% or higher per the total protein weight of the soluble protein extractable from the plant (or specific plant part, such as a bean).

In some embodiments, a transformed plant comprises in its genome: a recombinant DNA construct encoding a first protein and a second protein, wherein the first protein and/or the second protein is a milk protein. In some embodiments, a transformed plant comprises in its genome: a recombinant DNA construct encoding a first protein and a second protein, wherein the first protein is a milk protein and the second protein is a non-milk protein. In some embodiments, a transformed plant comprises in its genome a recombinant DNA construct encoding a fusion protein, wherein the fusion protein comprises a first protein and a second protein, wherein the first protein and the second protein are milk proteins. In some embodiments, a transformed plant comprises in its genome a recombinant DNA construct encoding a fusion protein, wherein the fusion protein comprises from N-terminus to C-terminus, the first protein and the second protein. In some embodiments, the fusion protein comprises, from N-terminus to C-terminus, the second protein and the first protein.

In some embodiments, a transformed plant comprises in its genome: a recombinant DNA construct encoding a fusion protein, wherein the fusion protein comprises (i) a milk protein, and (ii) an animal (e.g., mammalian or avian) protein. In some embodiments, a transformed plant comprises in its genome a recombinant DNA construct encoding a fusion protein, wherein the fusion protein comprises from N-terminus to C-terminus, the milk protein and the animal (e.g., mammalian or avian) protein. In some embodiments, the fusion protein comprises, from N-terminus to C-terminus, the animal (e.g., mammalian or avian) protein and the milk protein.

In some embodiments, a transformed plant comprises in its genome: a recombinant DNA construct encoding a fusion protein, wherein the fusion protein comprises a milk protein such as a casein protein. In some embodiments, a transformed plant comprises in its genome: a recombinant DNA construct encoding a fusion protein, wherein the fusion protein comprises a milk protein selected from α-S1 casein, α-S2 casein, β-casein, and κ-casein. In some embodiments, the milk protein is α-S1 casein. In some embodiments, the milk protein is α-S1 casein and comprises the sequence SEQ ID NO: 8, or a sequence at least 90% identical thereto. In some embodiments, the milk protein is α-S2 casein. In some embodiments, the milk protein is α-S2 casein and comprises the sequence SEQ ID NO: 84, or a sequence at least 90% identical thereto. In some embodiments, the milk protein is β-casein. In some embodiments, the milk protein is β-casein and comprises the sequence of SEQ ID NO: 6, or a sequence at least 90% identical thereto. In some embodiments, the milk protein is κ-casein. In some embodiments, the milk protein is κ-casein and comprises the sequence of SEQ ID NO: 4, or a sequence at least 90% identical thereto. In some embodiments, the milk protein is para-κ-casein. In some embodiments, the milk protein is para-κ-casein and comprises the sequence of SEQ ID NO: 2, or a sequence at least 90% identical thereto. In some embodiments, the milk protein is β-lactoglobulin. In some embodiments, the milk protein is β-lactoglobulin and comprises the sequence of SEQ ID NO: 10, or a sequence at least 90% identical thereto. In some embodiments, the milk protein is α-lactalbumin, lysozyme, lactoferrin, lactoperoxidase, or an immunoglobulin (e.g., IgA, IgG, IgM, or IgE).

In some embodiments, a transformed plant comprises in its genome: a recombinant DNA construct encoding a fusion protein, wherein the fusion protein comprises a mammalian protein selected from hemoglobin, or collagen, IgM, or IgE. In some embodiments, a transformed plant comprises in its genome: a recombinant DNA construct encoding a fusion protein, wherein the fusion protein comprises an avian protein selected from lysozyme, ovalbumin, ovotransferrin, and ovoglobulin.

In some embodiments, a transformed plant comprises in its genome: a recombinant DNA construct encoding a fusion protein, wherein the fusion protein comprises a casein protein and β-lactoglobulin. In some embodiments, a transformed plant comprises in its genome: a recombinant DNA construct encoding a fusion protein, wherein the fusion protein comprises κ-casein and β-lactoglobulin. In some embodiments, the fusion protein comprises para-κ-casein and β-lactoglobulin. In some embodiments, the fusion protein comprises β-casein and 0-lactoglobulin. In some embodiments, the fusion protein comprises α-S1 casein and 0-lactoglobulin. In some embodiments, the fusion protein comprises two, three, four, five, or six β-caseins.

In some embodiments, a transformed plant comprises in its genome: a recombinant DNA construct encoding a fusion protein; wherein the fusion protein comprises (1) κ-casein, and (ii) β-lactoglobulin. In some embodiments the fusion protein is expressed in the plant in an amount of 1% or higher per the total protein weight of the soluble protein extractable from the plant.

In some embodiments, a transformed plant comprises in its genome: a recombinant DNA construct encoding a fusion protein, wherein the fusion protein comprises a first protein and a second protein, wherein the first protein and the second protein are each casein proteins. In some embodiments, the recombinant fusion protein comprises κ-casein and para-κ-casein. In some embodiments, the recombinant fusion protein comprises κ-casein and β-casein. In some embodiments, the recombinant fusion protein comprises κ-casein and α-S1-casein. In some embodiments, the recombinant fusion protein comprises κ-casein and α-S2-casein. In some embodiments, the recombinant fusion protein comprises para-κ-casein and β-casein. In some embodiments, the recombinant fusion protein comprises para-κ-casein and α-S1-casein. In some embodiments, the recombinant fusion protein comprises para-κ-casein and α-S2-casein. In some embodiments, the recombinant fusion protein comprises β-casein and α-S1-casein. In some embodiments, the recombinant fusion protein comprises β-casein and α-S2-casein. In some embodiments, the recombinant fusion protein comprises α-S1-casein and α-S2-casein.

In some embodiments, the recombinant fusion protein comprises two or more of the same casein proteins. In some embodiments, the recombinant fusion protein comprises κ-casein and κ-casein. In some embodiments, the recombinant fusion protein comprises β-casein and β-casein. In some embodiments, the recombinant fusion protein comprises para-κ-casein and para-κ-casein. In some embodiments, the recombinant fusion protein comprises α-S1-casein and α-S1-casein. In some embodiments, the recombinant fusion protein comprises α-S2-casein and α-S2-casein.

In some embodiments, the transformed plant is a monocot. For example, in some embodiments, the plant may be a monocot selected from turf grass, maize (corn), rice, oat, wheat, barley, sorghum, orchid, iris, lily, onion, palm, and duckweed.

In some embodiments, the transformed plant is a dicot. For example, in some embodiments, the plant may be a dicot selected from Arabidopsis, tobacco, tomato, potato, sweet potato, cassava, alfalfa, lima bean, pea, chickpea, soybean, carrot, strawberry, lettuce, oak, maple, walnut, rose, mint, squash, daisy, Quinoa, buckwheat, mung bean, cow pea, lentil, lupin, peanut, fava bean, French beans (i.e., common beans), mustard, or cactus. In some embodiments, the plant is a soybean (Glycine max).

In some embodiments, the plant is a non-vascular plant selected from moss, liverwort, hornwort or algae. In some embodiments, the plant is a vascular plant reproducing from spores (e.g., a fern).

In some embodiments, the recombinant DNA construct is codon-optimized for expression in the plant. For example, in some embodiments, the recombinant DNA construct is codon-optimized for expression in a soybean plant.

The transgenic plants described herein may be generated by various methods known in the art. For example, a nucleic acid encoding a fusion protein may be contacted with a plant, or a part thereof, and the plant may then be maintained under conditions wherein the fusion protein is expressed. In some embodiments, the nucleic acid is introduced into the plant, or part thereof, using one or more methods for plant transformation known in the art, such as Agrobacterium-mediated transformation, particle bombardment-medicated transformation, electroporation, and microinjection.

In some embodiments, a method for stably expressing a recombinant fusion protein in a plant comprises (i) transforming a plant with a plant transformation vector comprising an expression cassette comprising: a sequence encoding a fusion protein, wherein the fusion protein comprises a milk protein, and an animal (e.g., mammalian or avian) protein; and (ii) growing the transformed plant under conditions wherein the recombinant fusion protein is expressed. In some embodiments, the milk protein is κ-casein. In some embodiments, the animal protein is β-lactoglobulin. In some embodiments, the milk protein is κ-casein and the animal protein is β-lactoglobulin. In some embodiments, the recombinant fusion protein is expressed in an amount of 1% or higher per the total protein weight of the soluble protein extractable from the plant.

### Casein Accumulation in Plants

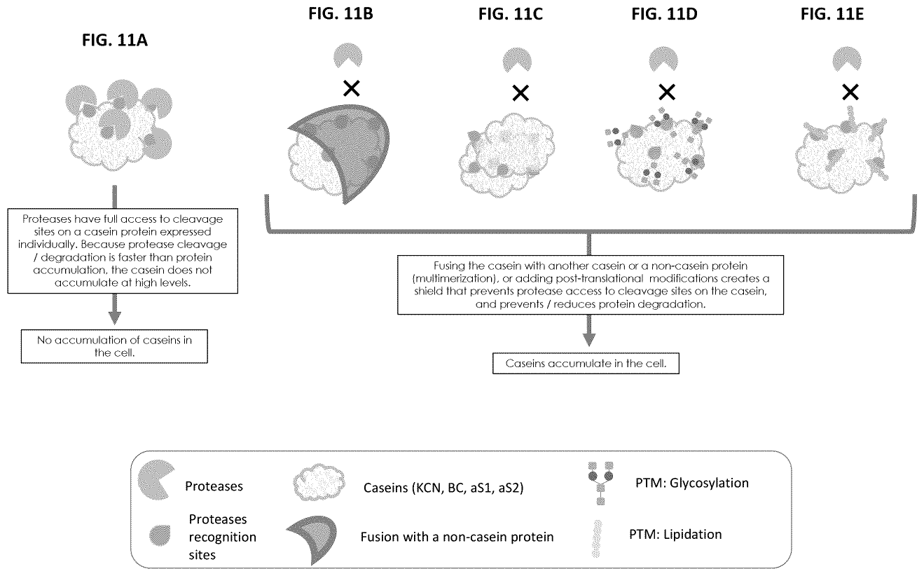

As described herein, fusion proteins comprising one or more milk proteins (e.g., casein proteins) accumulate to a greater extent in plant cells than the milk proteins expressed individually (not as fusion proteins). Caseins aggregate and bind to calcium-phosphate to form micelles. Without being bound by any theory, it is believed that native plant proteases are capable of degrading caseins by cleavage at various protease recognition sites (FIG. 11A). Thus, when caseins are expressed alone (i.e., not as a fusion protein), they are degraded quickly and do not accumulate in the cells. When caseins are fused to a second protein (FIG. 11B, FIG. 11C), the second protein may partially or fully limit protease access to the cleavage site on the caseins and may reduce degradation thereof. The extent of protection may vary depending on the properties of the second protein. For example, fusion proteins comprising two caseins (e.g., homodimers or heterodimers, FIG. 11C) may be able adopt a conformation that partially or fully prevents access to one or more protease cleavage sites. Some non-casein proteins, such as beta-lactoglobulin, GFP, or lysozyme, may also partially or fully block protease access, allowing casein accumulation at high levels in the cell (FIG. 11B). Without being bound by any theory, it is believed that fusion of a casein to a second protein comprising one, two or all three of the following characteristics is able to prevent access to one or more protease cleavage sites on the casein: (i) a molecular weight of 15 kDa or higher; (ii) at least 30% hydrophobic amino acids; and/or (iii) less than about 2.5 disulfide bonds per 10 kDa molecular weight.

Protease access to cleavage sites on a casein protein may also be blocked, for example, by the addition of one or more post-translational modifications to the casein, such as phosphorylation, glycosylation (FIG. 11D) or lipidation (FIG. 11E). Thus, in some embodiments, a recombinant casein protein described herein comprises one or more post-translational modifications. The post-translational modifications may, in some embodiments, prevent proteolysis by endogenous plant proteases. For example, the presence of one or more post-translational modifications on a recombinant casein may reduce proteolysis of the casein in a plant cell by at least 10%, at least 20%, at least 30%, at least 40%, at least 50%, at least 60%, at least 70%, at least 80%, at least 90%, at least 100%, at least 200% or more, relative to the proteolysis of a casein that does not have the one or more post-translational modifications. In some embodiments, the presence of one or more post-translational modifications on a recombinant casein may lead to an increase in expression of at least 2-fold, at least 3-fold, at least 4-fold, at least 5-fold, at least 10-fold, at least 20-fold, at least 30-fold, at least 40-fold, at least 50-fold or more, relative to the expression of a casein that does not have the one or more post-translational modifications. The recombinant casein proteins comprising post-translational modifications described herein may be expressed alone or may be expressed in a fusion protein (e.g., a casein protein homo- or hetero-multimer).

In some embodiments, the post-translational modifications may be non-mammalian post-translational modifications. For example, the post-translational modifications may be plant post-translational modifications. In some embodiments, the post-translational modifications may not typically occur in a casein protein when expressed in a plant or an animal cell. A non-limiting list of post-translational modifications that may be used to prevent proteolysis by endogenous plant proteases includes glycosylation (e.g., 0-glycans, N-glycans, or glycosaminoglycans such as heparin, heparan sulfate, chondroitin sulfate, keratan sulfate or dermatan sulfate), phosphorylation, lipidation, ubiquitylation, nitrosylation, methylation, acetylation, amidation, prenylation, alkylation, gamma-carboxylation, biotinylation, oxidation, or sulfation. In some embodiments, the post-translational modification is phosphorylation.

In some embodiments, a recombinant milk protein (e.g., a casein protein) comprises a site for post-translational modification that is not present in the native form of the protein. In some embodiments, a recombinant milk protein (e.g., a casein protein) comprises at least one, at least two, at least three, at least four, at least five, at least six, at least seven, at least eight, at least nine, or more sites for post-translational modifications that are not present in the native form of the protein. In some embodiments, a recombinant milk protein (e.g., a casein protein) comprises at least one, at least two, at least three, at least four, at least five, at least six, at least seven, at least eight, at least nine, or more post-translational modifications at sites that are not present in the native form of the protein.

In some embodiments, a recombinant milk protein (e.g., a casein protein) comprises an amino acid sequence that is modified to promote addition of one or more post-translational modifications in a plant cell. In some embodiments, the one or more post-translational modifications are selected from glycosylation, phosphorylation, lipidation, ubiquitylation, nitrosylation, methylation, acetylation, amidation, prenylation, alkylation, gamma-carboxylation, biotinylation, oxidation, and sulfation. In some embodiments, the amino acid sequence of a recombinant casein protein may be modified to introduce one or more glycosylation or phosphorylation sites.

In some embodiments, a milk protein is expressed in a plant, wherein the milk protein comprises an amino acid sequence that is modified to promote addition of one or more post-translational modifications, and wherein the milk protein comprises one or more post-translational modifications that are not present in a non-modified milk protein expressed in the same type of plant. In some embodiments, the milk protein is expressed in a plant in an amount of 1% or higher per total protein weight of soluble protein extractable from the plant. In some embodiments, the milk protein is a casein protein selected from α-S1 casein, α-S2 casein, (3-casein, κ-casein, and para-κ-casein.

In some embodiments, a fusion protein comprises (i) a recombinant milk protein that comprises an amino acid sequence that is modified to promote addition of one or more post-translational modifications in a plant cell, and (ii) at least one additional protein. In some embodiments, the at least one additional protein is a milk protein. In some embodiments, the at least one additional protein is a casein protein selected from α-S1 casein, α-S2 casein, (3-casein, κ-casein, and para-κ-casein. In some embodiments, the at least one additional protein is β-lactoglobulin. In some embodiments, the recombinant milk protein is κ-casein or para-κ-casein and the at least one additional protein is β-lactoglobulin. In some embodiments, the recombinant milk protein is β-casein and the at least one additional protein is β-lactoglobulin. In some embodiments, the recombinant milk protein is α-S1 casein or α-S2 casein and the at least one additional protein is β-lactoglobulin. In some embodiments, the fusion protein is expressed in a plant in an amount of 1% or higher per total protein weight of soluble protein extractable from the plant. In some embodiments, the plant is soybean.

In some embodiments, a transgenic plant expresses a milk protein comprising an amino acid sequence that is modified to promote addition of one or more post-translational modifications, or a fusion protein comprising the same.

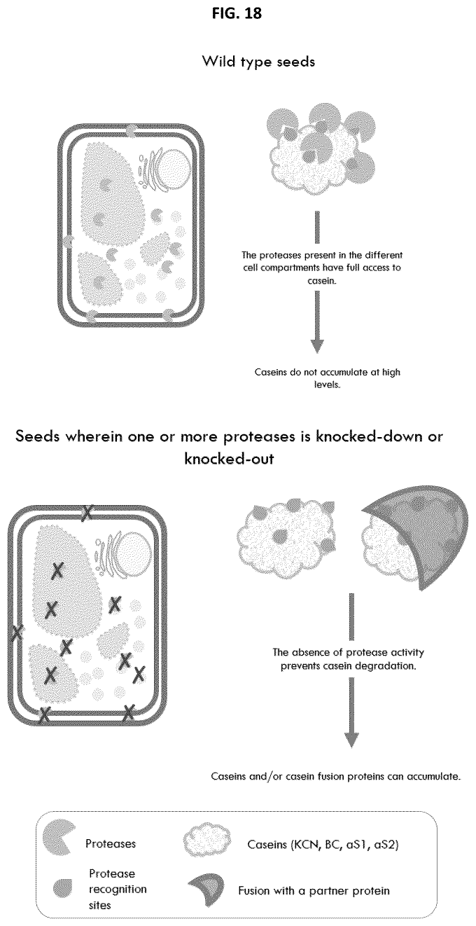

Proteolysis of recombinant caseins in plant cells may also be prevented by modifying the plant cell itself. Without being bound by any theory, it is believed that in wildtype seeds, proteases present in one or more cellular compartments may bind to and cleave casein expressed therein. Thus, casein does not accumulate at high levels in the seeds (See FIG. 18, top panel). In contrast, when expression of one or more proteases is knocked-down or knocked-out in the seed (indicated by “X” in the bottom panel of FIG. 18), degradation of the casein is substantially prevented. Accordingly, the casein can accumulate in the seed. This strategy may be used to increase expression in the seed of casein monomers (i.e., caseins expressed alone, not as a fusion protein), or fusion proteins comprising one or more caseins.

In some embodiments, expression of one or more endogenous plant proteases may be knocked down or knocked out in a plant cell (e.g., a seed). The one or more proteases may be, for example, one or more proteases endogenously expressed in a plant (e.g., a soybean), such as cysteine proteases, serine proteases, threonine proteases, or aspartic proteases, glutamic protases, metalloproteases, or asparagine peptide lyases. A non-limiting list of genes encoding proteases that may be knocked down or knocked out in a plant cell is provided below in Table 10. Additional proteases that may be knocked down or knocked out in a soybean cell are described in Shamimuzzaman M., Vodkin L (2018) Ribosome profiling reveals changes in translational status of soybean transcripts during immature cotyledon development. PLoS ONE 13(3): e0194596.

In some embodiments, expression of at least one, at least two, at least three, at least four, at least five, at least six, at least seven, at least eight, at least nine, at least ten or more proteases may be knocked down or knocked out in a plant cell.

TABLE 10
Genes encoding proteases that are transcriptionally active in soybeans
Soybean Gene ID

Glyma.02g213000
Glyma.03g125400
Glyma.03g239700
Glyma.04g022500
Glyma.04g027600
Glyma.04g091800
Glyma.06g022600
Glyma.06g027700
Glyma.06G272700
Glyma.06g275300
Glyma.08g116300
Glyma.08G116400
Glyma.09g187200
Glyma.09g226700
Glyma.09g249500
Glyma.10g207100
Glyma.12G010100
Glyma.13g027600
Glyma.13g196200
Glyma.13g208200
Glyma.13g255900
Glyma.13g321700
Glyma.14g048000
Glyma.14g064600
Glyma.14g085800
Glyma.14g216300
Glyma.15g177800
Glyma.15g234300
Glyma.16G018900
Glyma.17g164100
Glyma.17g239000
Glyma.17g254900
Glyma.18G242900
Glyma.18g250100
Glyma.19G236600

TABLE 11
Proteases that may be knocked down or knocked out in a plant cell

| | Accession No. | DNA | Protein |
|---|---|---|---|
| Protein Name | (Uniprot) | Sequence | Sequence |
| Peptidase A1 domain- | Glyma.04g091800 | 851 | 852 |
| containing protein | | | |
| Cysteine proteinase | Glyma.10g207100 | 853 | 854 |
| 34 kDa maturing seed | Glyma.08g116300 | 855 | 856 |
| protein | | | |
| Uncharacterized protein | Glyma.06g275300 | 857 | 858 |
| (cysteine protease family | | | |
| C1-related) | | | |
| Uncharacterized protein | Glyma.17g164100 | 859 | 560 |
| (Subsilin-like serine | | | |
| peptidase) | | | |

In some embodiments, a plant cell for expressing recombinant milk proteins is provided, wherein expression of one or more proteases is reduced (e.g., knocked down or knocked out) in the cell. The expression of the one or more proteases may be reduced (e.g., knocked down or knocked out), for example, using a gene editing technology (e.g., CRISPR, TALENs, Zn Finger Nuclease, etc.) or base editing technology (e.g., using a cytidine deaminase or an adenosine deaminase). In some embodiments, expression of the one or more proteases may be reduced using RNA interference (e.g., microRNAs or siRNAs). In some embodiments the one or more proteases that is knocked down or knocked out is a cysteine protease, a serine protease, or an aspartyl protease. In some embodiments, the one or more proteases that is knocked down or knocked out is any one of the proteases listed in Table 10 or Table 11. In some embodiments, the one or more proteases that is knocked down or knocked out comprises the sequence of any one of SEQ ID NO: 852, 584, 856, 858, or 860. In some embodiments, the one or more proteases that is knocked down or knocked out comprises a sequence with at least 85%, at least 90%, at least 91%, at least 92%, at least 93%, at least 94%, at least 95%, at least 96%, at least 97%, at least 98%, or at least 99% sequence identity with any one of SEQ ID NO: 852, 584, 856, 858, or 860. In some embodiments, the one or more proteases that is knocked down or knocked out comprises a sequence of any one of SEQ ID NO: 852, 584, 856, 858, or 860 plus at least 1, at least 2, at least 3, at least 4, at least 5, at least 6, at least 7, at least 8, at least 9, at least 10, at least 11, at least 12, at least 13, at least 14, at least 15, or more amino acid substitutions. The expression or activity of endogenous plant proteases may also be reduced using small molecule inhibitors thereof (i.e., protease inhibitors).

Also provided is a transgenic plant comprising a plant cell for expressing recombinant milk proteins, wherein expression of one or more proteases is reduced (e.g., knocked down or knocked out) in the plant.

In some embodiments, a method for stably expressing a recombinant milk protein in a plant comprises: (i) reducing expression of one or more proteases in the plant, (ii) transforming the plant with a plant transformation vector comprising an expression cassette encoding a recombinant milk protein or a fusion protein comprising the same, (iii) growing the transformed plant under conditions wherein the recombinant milk protein is expressed in an amount of 1% or higher per total weight of soluble protein extractable from the plant.

In some embodiments, a recombinant casein protein that comprises one or more post-translational modifications is produced in a plant cell by expressing or over-expressing one or more enzymes in the plant cell, such as an enzyme known to perform post-translational modifications (e.g., a kinase, a phosphatase, or glycosyltransferase). In some embodiments, a recombinant casein protein that comprises one or more post-translational modifications is produced in a plant cell by knocking out or knocking down one or more enzymes the plant cell known to remove or prevent addition of post-translational modifications (e.g., a phosphatase or an endoglycosidase). In some embodiments, a recombinant casein protein that comprises one or more post-translational modifications is produced in a plant cell by contacting the cell with one or more precursors of the post-translational modification (e.g., a nucleotide sugar precursor).

In some embodiments, a recombinant casein protein comprises one or more glycoprotein tags. For example, in some embodiments, a recombinant casein protein may comprise a glycoprotein tag derived from a hydroxyproline (Hyp)-rich glycoprotein (GRGP). In some embodiments, the glycoprotein tag comprises SP repeats. For example, the glycoprotein tag may be derived from a glycoprotein comprising 11 tandem SP repeats (See Glyma.02 g204500, annotated as early nodulin-like protein 10 in soy). In some embodiments, the fusion protein comprises the M domain of CD45 (receptor-type tyrosine-protein phosphatase C), or a fragment or derivative thereof. For example, in some embodiments, the fusion protein comprises amino acids Ala231 to Asp 290 of Uniprot Accession No. P08575. In some embodiments, the glycoprotein tag comprises the sequence of SEQ ID NO: 824. In some embodiments, the glycoprotein tag is encoded by the sequence SEQ ID NO: 825. In some embodiments, the glycoprotein tag comprises the sequence of SEQ ID NO: 827. In some embodiments, the glycoprotein tag is encoded by the sequence of SEQ ID NO: 826. The glycoprotein tag may be fused, in some embodiments, to the N-terminus or the C-terminus of the casein protein.

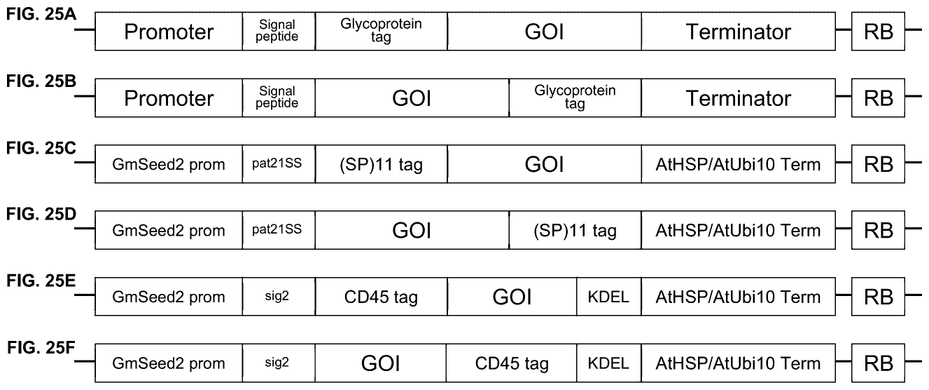

Illustrative expression cassettes for expressing a gene of interest (GOI; e.g., a casein) fused to a glycoprotein tag are provided in FIG. 25A-25F. In some embodiments, an expression cassette comprises a promoter, a signal peptide, a glycoprotein tag, a GOI (e.g., a casein) and a terminator (See FIG. 25A). In some embodiments, an expression cassette comprises a promoter, a signal peptide, a GOI, a glycoprotein tag, and a terminator. (See FIG. 25B). In some embodiments, an expression cassette comprises the GmSeed 2 promoter (SEQ ID NO: 813), the pat21ss signal peptide (SEQ ID NO: 823), a (SP)11 glycoprotein tag (SEQ ID NO: 825), a GOI (e.g., a casein) and the AtHSP/AtUBi10 Terminator (SEQ ID NO: 815, 816) (See FIG. 25C). In some embodiments, an expression cassette comprises the GmSeed 2 promoter (SEQ ID NO: 813), the pat21ss signal peptide (SEQ ID NO: 823), a GOI (e.g., a casein), a (SP)11 glycoprotein tag (SEQ ID NO: 825), and the AtHSP/AtUBi10 Terminator (SEQ ID NO: 815,816) (See FIG. 25D). In some embodiments, an expression cassette comprises the GmSeed 2 promoter (SEQ ID NO: 813), the sig2 signal peptide (SEQ ID NO: 814), a CD45 tag (SEQ ID NO: 827), a GOI (e.g., a casein), a KDEL sequence, and the AtHSP/AtUBi10 Terminator (SEQ ID NO: 815, 816) (See FIG. 25E). In some embodiments, an expression cassette comprises the GmSeed 2 promoter (SEQ ID NO: 813), the sig2 signal peptide (SEQ ID NO: 814), a GOI (e.g., a casein), a CD45 tag (SEQ IDNO: 827), a KDEL sequence, and the AtHSP/AtUBi10 Terminator (SEQ ID NO: 815, 816) (See FIG. 25F).

Following protein synthesis, many eukaryotic proteins undergo post-translational modification (PTM). These modifications may be for example, the covalent addition of a function group, and contributes to protein diversity and function. Examples of PTMs include, but are not limited to, phosphorylation, glycosylation, ubiquitination, nitrosylation, methylation, acetylation, and lipidation. The proteins within milk also undergo PTM (Greenberg et al., “Human beta-casein. Amino acid sequence and identification of phosphorylation sites,” J. Biol. Chem., 1984, 259(8):5132-5138, Imafidon et al., “Isolation, purification, and alteration of some functional groups of major milk proteins: a review,” Crit. Rev. Food. Sci. Nutr. 37(7):663-689, 1997). For example, alpha and beta caseins are phosphorylated, and kappa casein is glycosylated. It has been reported that caseins assemble in a colloidal complex with calcium phosphate and other minerals.

In some embodiments, a casein protein expressed in a plant cell comprises different post-translational modifications relative to the same casein protein expressed by a mammalian cell. In some embodiments, a casein protein expressed in a plant cell does not comprise any post-translational modifications. In some embodiments, a casein protein expressed in a plant cell has reduced phosphorylation compared to the same casein protein expressed in a mammalian cell. In some embodiments, a casein protein expressed in a plant cell has increased phosphorylation compared to the same casein protein expressed in a mammalian cell.

In some embodiments, the compositions and methods described herein can be used to produce a casein protein that does not comprise any post-translational modifications. In some embodiments, the compositions and methods described herein can be used to produce a casein protein that is substantially free of phosphorylation. In some embodiments, the compositions and methods described herein can be used to produce a casein protein in a plant cell that comprises substantially the same level of post-translational modifications relative to the same casein protein expressed in a mammalian cell. In some embodiments, the compositions and methods described herein can be used to produce a casein protein that comprises substantially the same level of phosphorylation relative to the same casein protein expressed in a mammalian cell. For example, in some embodiments, a casein protein expressed in a plant cell may comprise at least 50%, at least 60%, at least 70%, at least 80%, at least 90%, or at least 95% of the number of phosphates relative to the same casein protein expressed in a mammalian cell.

### Methods for Producing Recombinant Milk Proteins, Including Casein Proteins

The recombinant milk proteins (e.g., casein proteins) described herein may be produced in a number of non-mammalian species, including for example, plants and microorganisms such as yeast and bacteria.

The recombinant casein proteins may be expressed in one or more non-mammalian cells using genetic sequences (e.g., DNA or RNA sequences) isolated or derived from cow (Bos taurus), goat (Capra hircus), sheep (Ovis aries), water buffalo (Bubalus bubalis), dromedary camel (Camelus dromedaries), bactrian camel (Camelus bactrianus), wild yak (Bos mutus), horse (Equus caballus), donkey (Equus asinus), reindeer (Rangifer tarandus), Eurasian elk (Alces alces), alpaca (Vicugna pacos), zebu (Bos indicus), llama (Lama glama), or human (Homo sapiens). In some embodiments, a genetic sequence used to encode the recombinant casein has at least 80%, at least 85%, at least 90%, at least 95%, at least 96%, at least 97%, at least 98%, or at least 99% identity with the genetic sequence sued to encode a casein protein in one or more of cow (Bos taurus), goat (Capra hircus), sheep (Ovis aries), water buffalo (Bubalus bubalis), dromedary camel (Camelus dromedaries), bactrian camel (Camelus bactrianus), wild yak (Bos mutus), horse (Equus caballus), donkey (Equus asinus), reindeer (Rangifer tarandus), eurasian elk (Alces alces), alpaca (Vicugna pacos), zebu (Bos indicus), llama (Lama glama), or human (Homo sapiens). In some embodiments, the recombinant casein protein expressed in a non-mammalian cell has at least 80%, at least 85%, at least 90%, at least 95%, at least 96%, at least 97%, at least 98%, or at least 99% identity with a casein protein from one or more of cow (Bos taurus), goat (Capra hircus), sheep (Ovis aries), water buffalo (Bubalus bubalis), dromedary camel (Camelus dromedaries), bactrian camel (Camelus bactrianus), wild yak (Bos mutus), horse (Equus caballus), donkey (Equus asinus), reindeer (Rangifer tarandus), eurasian elk (Alces alces), alpaca (Vicugna pacos), zebu (Bos indicus), llama (Lama glama), or human (Homo sapiens).

When expressed in a plant, the recombinant casein proteins may be extracted using standard methods known in the art. For example, the casein proteins may be extracted using solvent or aqueous extraction or using phenol extraction. Once extracted, the casein proteins may be maintained in a buffered environment (e.g., Tris, MOPS, HEPES), in order to avoid sudden changes in the pH. The casein proteins may also be maintained at a particular temperature, such as 4° C. One or more additives may be used to aid the extraction process (e.g., salts, protease/peptidase inhibitors, osmolytes, reducing agents, etc.).

### Protein Co-Expression in Plants

Another way to increase accumulation of one or more recombinant proteins, such as milk proteins, in a plant cell is to co-express the protein with a second protein, such as a protein capable of forming a protein body (e.g., a prolamin). Without being bound by any theory, it is believed that co-expressing a milk protein and a prolamin protein in a plant cell will cause protein body formation in the plant cell, wherein the milk protein gets sequestered into and/or associated with the protein body. This protects the milk protein from degradation by one or more proteases and increases accumulation thereof in the plant cell.

In some embodiments, two or more recombinant proteins may be co-expressed in a plant cell. In some embodiments, one of the two or more recombinant proteins is a milk protein (e.g., casein protein). In some embodiments, the milk protein is selected from the group consisting of: α-S1 casein, α-S2 casein, β-casein, κ-casein, para-κ-casein, β-lactoglobulin, α-lactalbumin, lysozyme, lactoferrin, lactoperoxidase, and an immunoglobulin. In some embodiments, the milk protein is β-casein or β-lactoglobulin.

In some embodiments, one of the two or more proteins is a protein capable of forming a protein body. For example, in some embodiments, one of the two or more proteins is a prolamin (e.g., zein and/or canein). In some embodiments, the prolamin is selected from the group consisting of: gliadin, a hordein, a secalin, a zein, a kafirin, and an avenin. In some embodiments, the protein capable of forming a protein body is a hydrophobin or an elastin-like protein. In some embodiments, at least two proteins are co-expressed in a plant cell (e.g., a casein protein and a prolamin). In some embodiments, the at least two proteins are casein and zein (e.g., gamma-zein). In some embodiments, the at least two proteins are casein and canein.

In some embodiments, a method for expressing a first recombinant protein in a cell comprises: (i) contacting the cell with a vector encoding a first recombinant protein, and (ii) contacting the cell with a vector encoding a second recombinant protein, wherein the second recombinant protein is capable of forming a protein body (e.g., a prolamin.) In some embodiments, the first recombinant protein is a casein protein, such as a milk protein.

A milk protein (e.g., a casein protein) may, in some embodiments, be co-expressed with a protein capable of forming a protein body (e.g., a prolamin) in a transgenic plant. In some embodiments, co-expressing a milk protein (e.g., a casein protein) with a protein capable of forming a protein body (e.g., a prolamin) in a transgenic plant leads to accumulation of the milk protein in an amount of at least 1%, at least 1.5%, at least 2%, at least 2.5%, at least 3%, at least 3.5%, at least 4%, at least 4.5%, at least 5%, at least 5.5%, at least 6%, at least 6.5%, at least 7%, at least 7.5%, at least 8%, at least 8.5%, at least 9%, at least 9.5%, at least 10%, at least 10.5%, at least 11%, at least 11.5%, at least 12%, at least 12.5%, at least 13%, at least 13.5%, at least 14%, at least 14.5%, at least 15%, at least 15.5%, at least 16%, at least 16.5%, at least 17%, at least 17.5%, at least 18%, at least 18.5%, at least 19%, at least 19.5%, at least 20%, or more of total protein weight of soluble protein extractable from the plant.

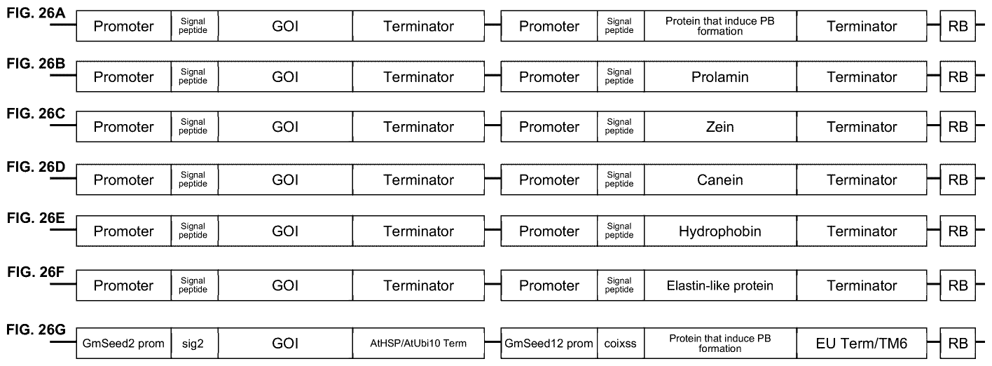

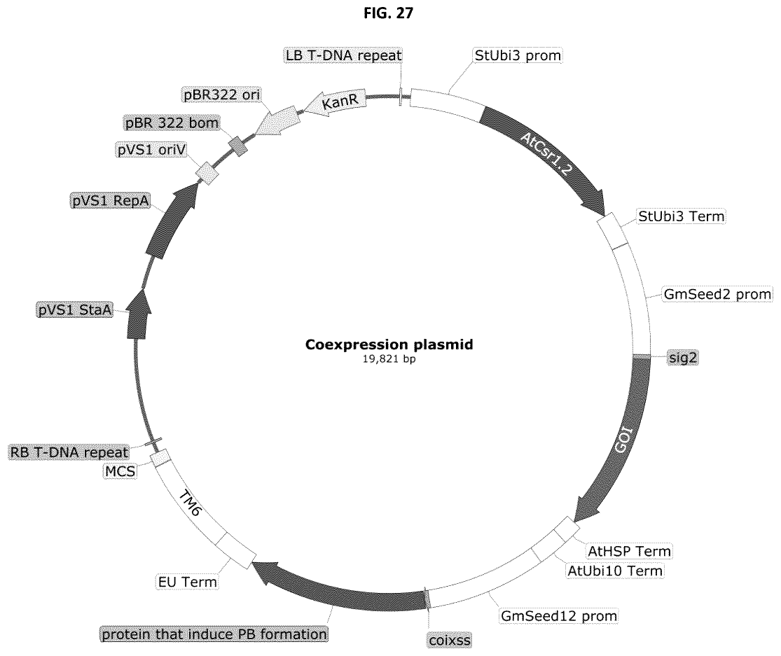

Illustrative constructs for co-expressing a milk protein (e.g., a casein protein) and a protein capable of inducing formation of a protein body in a plant cell are provided in FIG. 26A-26G. In some embodiments, a construct comprises (i) a first expression cassette comprising a promoter, a signal peptide, a Gene of Interest (e.g., a casein protein) and a terminator, and (ii) a second expression cassette comprising a promoter, a signal peptide, a protein that induces protein body formation, and a terminator (See FIG. 26A). In some embodiments, a construct comprises (i) a first expression cassette comprising a promoter, a signal peptide, a Gene of Interest (e.g., a casein protein) and a terminator, and (ii) a second expression cassette comprising a promoter, a signal peptide, a prolamin, and a terminator (See FIG. 26B). In some embodiments, a construct comprises (i) a first expression cassette comprising a promoter, a signal peptide, a Gene of Interest (e.g., a casein protein) and a terminator, and (ii) a second expression cassette comprising a promoter, a signal peptide, a zein, and a terminator (See FIG. 26C). In some embodiments, a construct comprises (i) a first expression cassette comprising a promoter, a signal peptide, a Gene of Interest (e.g., a casein protein) and a terminator, and (ii) a second expression cassette comprising a promoter, a signal peptide, a canein, and a terminator (See FIG. 26D). In some embodiments, a construct comprises (i) a first expression cassette comprising a promoter, a signal peptide, a Gene of Interest (e.g., a casein protein) and a terminator, and (ii) a second expression cassette comprising a promoter, a signal peptide, a hydrophobin, and a terminator (See FIG. 26E). In some embodiments, a construct comprises (i) a first expression cassette comprising a promoter, a signal peptide, a Gene of Interest (e.g., a casein protein) and a terminator, and (ii) a second expression cassette comprising a promoter, a signal peptide, an elastin-like protein, and a terminator (See FIG. 26F). In some embodiments, a construct comprises (i) a first expression cassette comprising a GmSeed2 promoter, a Sig2 signal peptide, a Gene of Interest (e.g., a casein protein) and a AtHSP/AtUbi10 terminator, and (ii) a second expression cassette comprising a GmSeed 12 promoter, a Coixss signal peptide, a protein that induces protein body formation, and a EU Term/Tm6 terminator (See FIG. 26G). An illustrative binary vector for use in co-expressing a casein and a protein that can induce protein body formation is provided in FIG. 27.

In some embodiments, a milk protein (e.g., a casein protein) can be co-expressed with one or more proteins capable of adding or removing a post-translational modification to/from a milk protein. For example, in some embodiments, the milk protein may be co-expressed with one or more of a kinase, a phosphatase, or a glycosyltransferase. In some embodiments, the milk protein is co-expressed with a kinase. The kinase may be for example, a kinase that phosphorylates Ser-X-Glu/pSer motifs. In some embodiments, the kinase may be a kinase in the family 20C, such as the Fam20C kinase. In some embodiments, the kinase may be a fragment or derivative of the Fam20C kinase, such as a truncated Fam20C comprising amino acids 94-586 of the native protein. In some embodiments, the kinase comprises amino acids 94-586 of SEQ ID NO: 821, or a sequence at least 90%, at least 95%, at least 96%, at least 97%, at least 98%, or at least 99% identical thereto. In some embodiments, the kinase is encoded by the sequence of SEQ ID NO: 820, or a sequence at least 90%, at least 95%, at least 96%, at least 97%, at least 98%, or at least 99% identical thereto.

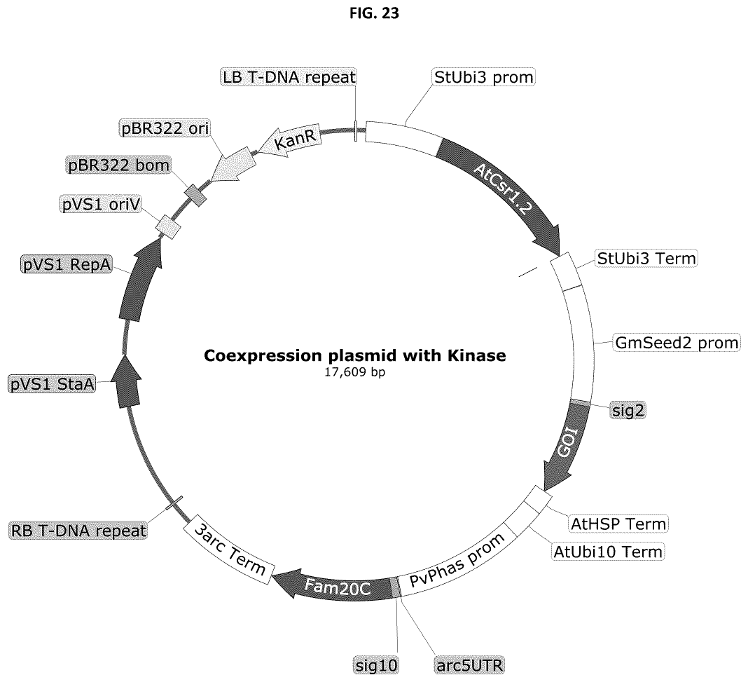

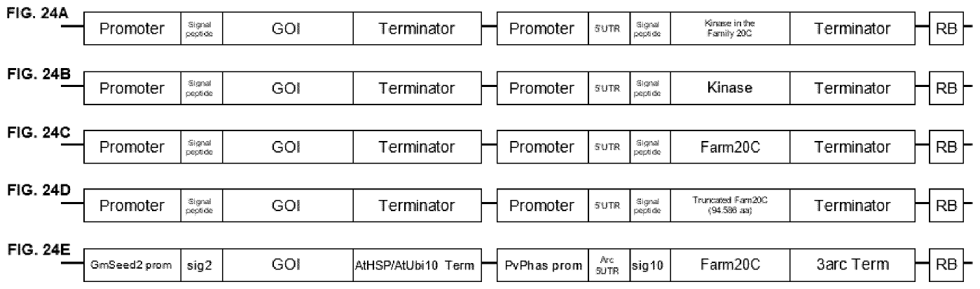

Illustrative expression cassettes that may be used to co-express a milk protein (e.g., a casein protein) with a kinase (or other enzyme capable of adding/removing a PTM) are shown in FIG. 24A-24E. In some embodiments, a construct for co-expression of a milk protein in a cell comprises: (i) a first expression cassette comprising a promoter, a signal peptide, a Gene of Interest (GOI, e.g., a casein protein) and a terminator, and (ii) a second expression cassette comprising a promoter, a 5′UTR, a signal peptide, a Gene of Interest (GOI, e.g., a kinase), and a terminator (See FIG. 24B). In some embodiments, a construct for co-expression of a milk protein in a cell comprises: (i) a first expression cassette comprising a promoter, a signal peptide, a Gene of Interest (GOI, e.g., a casein protein) and a terminator, and (ii) a second expression cassette comprising a promoter, a 5′UTR, a signal peptide, a Gene of Interest (GOI, e.g., a kinase in the 20C family), and a terminator (See FIG. 24A). In some embodiments, a construct for co-expression of a milk protein in a cell comprises: (i) a first expression cassette comprising a promoter, a signal peptide, a Gene of Interest (GOI, e.g., a casein protein) and a terminator, and (ii) a second expression cassette comprising a promoter, a 5′UTR, a signal peptide, a Gene of Interest (GOI, e.g., a Fam20C kinase), and a terminator (See FIG. 24C). In some embodiments, a construct for co-expression of a milk protein in a cell comprises: (i) a first expression cassette comprising a promoter, a signal peptide, a Gene of Interest (GOI, e.g., a casein protein) and a terminator, and (ii) a second expression cassette comprising a promoter, a 5′UTR, a signal peptide, a Gene of Interest (GOI, e.g., a truncated Fam20C kinase), and a terminator (See FIG. 24D). In some embodiments, the promoter may be the GmSeed2 promoter (SEQ ID NO: 813) or the PvPhas promoter (SEQ ID NO: 817). In some embodiments, the promoter may be the Sig2 signal peptide (SEQ ID NO: 814) or the sig10 signal peptide (SEQ ID NO: 819). In some embodiments, the terminator may be the AtHSP/AtUbi10 Terminator (SEQ ID NO: 815, 816) or the 3arc Terminator (SEQ ID NO: 822). In some embodiments, the 5′UTR may be the Arc 5′UTR (SEQ ID NO: 818). In some embodiments, the construct for co-expression of a milk protein in a cell comprises the construct of FIG. 24E. An illustrative binary vector is provided in FIG. 23.

In some embodiments, a milk protein (e.g., a casein protein) can be co-expressed with one or more proteins capable of inhibiting a protease. Illustrative plant proteins that may be used to inhibit one or more proteases are shown above in Table 4. In some embodiments, a milk protein may be co-expressed with any one of the proteins shown in Table 4. In some embodiments, a milk protein is co-expressed with a protein that comprises the sequence of any one of SEQ ID NO: 840, 842, 844, 846, 848 or 850. In some embodiments, a milk protein may be co-expressed with a protein having a sequence with at least 85%, at least 90%, at least 91%, at least 92%, at least 93%, at least 94%, at least 95%, at least 96%, at least 97%, at least 98%, or at least 99% identity to any one of SEQ ID NO: 840, 842, 844, 846, 848 or 850. In some embodiments, the milk protein may be co-expressed with a protein having the sequence of any one of SEQ ID NO: 840, 842, 844, 846, 848 or 850 plus at least 1, at least 2, at least 3, at least 4, at least 5, at least 6, at least 7, at least 8, at least 9, at least 10, at least 11, at least 12, at least 13, at least 14, at least 15, or more amino acid substitutions.

In some embodiments, protein co-expression can be utilized to reduce or prevent degradation of the one or more proteins in the plant cell, such as protease-mediated degradation in the plant cell. In some embodiments, the protein-co-expression is useful to reduce or prevent degradation of one or more milk proteins by proteases in a plant cell. In some embodiments co-expressing one or more milk proteins (e.g., casein protein) and a prolamin (e.g., a canein or a zein) may lead to the formation of a protein body in a seed of a plant. In some embodiments, the one or more milk proteins can be sequestered in and/or associated with the protein body, which in turn partially or fully shields the one or more milk proteins from degradation by plant cell proteases thereby allowing for accumulation of the one or more milk proteins. In some embodiments, the one or more milk proteins can be sequestered in the protein body, which in turn may protect a plant cell from potential toxic effects of recombinant proteins, such as any toxic effects of the one or more proteins.

In some embodiments, protein co-expression is effective in increasing at least one of concentration, stability, or expression of one or more proteins in a plant cell. In some embodiments, protein co-expression is effective in increasing concentration of one or more proteins in a plant cell as determined by detecting the amount of the one or more protein in the plant cell. In some embodiments, protein co-expression is effective in increasing stability of one or more proteins in a plant cell. Increased stability can be determined by detecting persistence of the one or more proteins in the plant cell over time or detecting a level of degradation. In some embodiments, protein co-expression is effective in increasing expression of one or more proteins in a plant cell. Increased expression can be determined by measuring protein level and/or accumulation in the plant cell. In some embodiments, protein co-expression is effective in increasing at least one of: concentration, stability, or expression of one or more proteins by at least about 1-fold, 10-fold, 19-fold, 28-fold, 37-fold, 46-fold, 55-fold, 64-fold, 73-fold, 82-fold, 91-fold, 100-fold, 109-fold, 118-fold, 127-fold, 136-fold, 145-fold, 154-fold, 163-fold, 172-fold, 181-fold, 190-fold, 199-fold, 208-fold, 217-fold, 226-fold, 235-fold, 244-fold, 253-fold, 262-fold, 271-fold, 280-fold, 289-fold, 298-fold, or up to about 300-fold as compared to an otherwise comparable method lacking the protein co-expression. In some embodiments, protein co-expression is effective in increasing at least one of concentration, stability, or expression of one or more proteins in a plant cell by at least about 1-fold to 10-fold, 5-fold to 30-fold, 20-fold to 50-fold, 40-fold to 100-fold, or 100-fold to 200-fold as compared to an otherwise comparable method lacking the protein co-expression.

In some embodiments, protein co-expression is effective in reducing toxicity of recombinant expression of the one or more proteins in a plant cell. In some embodiments, protein co-expression is effective in reducing toxicity of recombinant expression of one or more proteins in a plant cell by at least about 1-fold, 10-fold, 19-fold, 28-fold, 37-fold, 46-fold, 55-fold, 64-fold, 73-fold, 82-fold, 91-fold, 100-fold, 109-fold, 118-fold, 127-fold, 136-fold, 145-fold, 154-fold, 163-fold, 172-fold, 181-fold, 190-fold, 199-fold, 208-fold, 217-fold, 226-fold, 235-fold, 244-fold, 253-fold, 262-fold, 271-fold, 280-fold, 289-fold, 298-fold, or up to about 300-fold as compared to an otherwise comparable method lacking the protein co-expression. In some embodiments, protein co-expression is effective in reducing toxicity associated with recombinant expression of one or more proteins in a plant cell by at least about 1-fold to 10-fold, 5-fold to 30-fold, 20-fold to 50-fold, 40-fold to 100-fold, or 100-fold to 200-fold as compared to an otherwise comparable method lacking the protein co-expression.

In some embodiments, protein co-expression may be achieved via transformation of a composition comprising one or more vectors encoding the one or more proteins into a plant cell. In some embodiments, one or more vectors are binary agrobacterium vectors. In some embodiments, one or more vectors encodes for one or more protein sequences. In some embodiments, a single vector encodes for two or more protein sequences. In some embodiments, two or more vectors are used to introduced two or more sequences into a plant cell. In some embodiments, a vector encodes for a milk protein (e.g., casein protein) and a prolamin (e.g., a canein or a zein). In some embodiments, a vector encodes for a milk protein and a protein capable of forming a protein body. In some embodiments a first vector encodes for a milk protein and a second vector encodes for a prolamin. In some embodiments, a first vector encodes for a milk protein and a second vector encodes for a prolamin. Also provided are compositions that comprise one or more vectors described herein.

### Food Compositions Comprising a Fusion Protein or a Protein Derived Therefrom

The fusion proteins, recombinant proteins, and transgenic plants described herein may be used to prepare food compositions. The fusion protein may be used directly to prepare the food composition (i.e., used in the form of a fusion protein), or the fusion protein may first be separated into its constituent proteins. For example, in some embodiments, a food composition may comprise (i) a fusion protein, (ii) a milk protein (structured or unstructured) or (iii) a non-milk protein, such as a structured mammalian, avian, or plant protein.

More specifically, the present disclosure provides alternative dairy compositions, solid phase protein-stabilized emulsions (including cheese compositions), and colloidal suspensions, each comprising one or more casein proteins. The casein proteins may be isolated or recombinant and may be selected from the group consisting of kappa-casein, para-kappa-casein, beta-casein, alpha-S1-casein and alpha-S2-casein. The compositions, emulsions, or suspensions described herein may be used to produce food compositions (e.g., cheese, yogurt, ice cream, etc.) that have organoleptic properties similar to traditional animal-derived dairy compositions. For example, the food compositions described herein may have one or more characteristics of a traditional animal-derived dairy composition, such as taste, aroma, appearance, handling, mouthfeel, density, structure, texture, elasticity, springiness, coagulation, binding, leavening, aeration, foaming, creaminess and emulsification. The food compositions described herein offer a sustainable, environmentally-friendly, cruelty-free alternative to traditional animal-derived dairy compositions.

In some embodiments, the alternative dairy compositions, solid phase, protein-stabilized emulsions, and colloidal suspensions comprising recombinant casein proteins have non-mammalian PTMs. In some embodiments, the recombinant casein proteins are not phosphorylated or glycosylated. In some embodiments, the recombinant casein proteins have an alternative PTM pattern, as compared to naturally occurring casein proteins.

PTMs have been reported to be important for the casein micelle structure, which determines the physical properties of milk. Unexpectedly, the recombinant proteins described herein are still able to confer to the compositions described herein one or more organoleptic properties similar to animal-derived dairy compositions, such as taste, appearance, mouthfeel, structure, texture, density, elasticity, springiness, coagulation, binding, leavening, aeration, foaming, creaminess, and emulsification.

Food compositions, including alternative dairy compositions, solid phase protein-stabilized emulsions, and colloidal suspensions, are described in more detail below.

### Solid Phase Protein-Stabilized Emulsions

Provided herein are solid phase, protein-stabilized emulsions comprising at least one milk protein. For example, in some embodiments, a solid phase, protein-stabilized emulsion comprises at least one casein protein. In some embodiments, a protein-stabilized emulsion comprises at least one recombinant casein protein. In some embodiments, a protein-stabilized emulsion comprises at least one plant-expressed casein protein. In some embodiments, a protein-stabilized emulsion comprises at least one casein protein isolated from milk (e.g., bovine milk). In some embodiments, the protein-stabilized emulsion is a cheese composition.

In some embodiments, a solid-phase protein stabilized protein emulsion comprises only one casein protein. In some embodiments, the one casein protein is recombinant beta-casein protein.

In some embodiments, a solid-phase protein stabilized protein emulsion comprises only two casein proteins. In some embodiments, the two casein proteins are recombinant beta-casein protein and kappa-casein protein. In some embodiments, the two casein proteins are recombinant beta-casein protein and para-kappa-casein protein. In some embodiments, the two casein proteins are recombinant beta-casein protein and alpha-S1-casein protein. In some embodiments, the two casein proteins are recombinant beta-casein protein and alpha-S2-casein protein.

In some embodiments, a solid-phase, protein stabilized emulsion comprises only three casein proteins. In some embodiments, the three casein proteins are recombinant beta-casein, kappa-casein, and para-kappa-casein. In some embodiments, the three casein proteins are recombinant beta-casein, kappa-casein, and alpha-S1-casein. In some embodiments, the three casein proteins are recombinant beta-casein, kappa-casein, and alpha-S2-casein. In some embodiments, the three casein proteins are recombinant beta-casein, para-kappa-casein, and alpha-S1-casein. In some embodiments, the three casein proteins are recombinant beta-casein, para-kappa-casein, and alpha-S2-casein.

In some embodiments, a solid-phase, protein stabilized emulsion comprises only four casein proteins. In some embodiments, one of the four casein proteins is recombinant beta-casein.

The casein proteins used in the solid-phase, protein-stabilized emulsions described herein may be selected from kappa-casein, para-kappa-casein, beta-casein, alpha-S1-casein and alpha-S2-casein. In some embodiments, the solid-phase protein stabilized emulsions may comprise, in addition to the casein protein(s), one or more additional milk proteins. In some embodiments, the solid-phase protein stabilized emulsions may comprise, in addition to the casein protein(s), one or more plant proteins.

In some embodiments, the emulsion has a firmness of at least 150 grams. In some embodiments, the emulsion has a melting point of about 35° C. to about 100° C. In some embodiments, the emulsion has an ability to stretch to at least 3 cm in length without breaking. In some embodiments, the emulsion has a firmness of at least 150 grams and a melting point of about 35° C. to about 100° C. In some embodiments, the emulsion has a firmness of at least 150 grams and an ability to stretch to at least 3 cm in length without breaking. In some embodiments, the emulsion has a melting point of about 35° C. to about 100° C. and an ability to stretch to at least 3 cm in length without breaking. In some embodiments, the emulsion has a firmness of at least 150 grams, a melting point of about 35° C. to about 100° C., and an ability to stretch to at least 3 cm in length without breaking.

Firmness, also referred to herein as hardness, may be measured by a number of methods known in the art, such as by compression, or using an instrument such as the Instron Testing Machine (A. H. Chen et al., Textural analysis of cheese, 1979, J. Dariy Sci. 62:901-907). For example, a cylindrical-shaped sample of a solid-phase, protein stabilized emulsion may be compressed from 50% to 100% relative to its original height and/or width. The cylindrical shaped-sample may have a height in the range of about 1 to about 10 cm, or more, and a diameter in the range of about 1 to about 10 cm, or more. The compression may occur at a predetermined temperature, such as a temperature in the range of about 0° C. to about 5° C., about 5° C. to about 10° C., about 10° C. to about 20° C., about 15° C. to about 25° C., about 20° C. to about 25° C., about 25° C. to about 25° C. In some embodiments, firmness may be determined by compressing a cylindrical-shaped sample having a height of about 3 cm, and a diameter of about 3 cm may be compressed to a height of 1.5 cm at 5° C. The compositions described herein may have a firmness in the range of about 50 to 100 grams, about 100 to about 150 grams, about 150 grams to about 200 grams, about 200 to about 300 grams, about 300 grams to about 400 grams, about 400 grams to about 500 grams, about 500 grams to about 600 grams, about 600 grams to about 700 grams, about 700 grams to about 800 grams, about 800 grams to about 900 grams, about 900 grams to 1 kilogram, or more.

Stretch ability may be analyzed by standard assays known in the art. For example, stretch ability may be determined by heating a 100 gram mass of an emulsion at a temperature of 225° C. for 4 minutes, cooling to about 90° C., and then pulling with a fork placed beneath the mass. Other methods to test stretch ability are well known in the art. See for example, Fife R. L et al, Test for measuring the stretch ability of melted cheese, 2002, J. Dairy Sci. 85(12):3539-3545.

In some embodiments, the recombinant casein protein may be expressed by a plant (i.e., it is a “plant-expressed” protein). In some embodiments, the recombinant protein may be expressed in a monocot, such as turf grass, maize (corn), rice, oat, wheat, barley, sorghum, orchid, iris, lily, onion, palm, or duckweed. In some embodiments, the recombinant casein protein may be expressed in a dicot, such as Arabidopsis, tobacco, tomato, potato, sweet potato, cassava, alfalfa, lima bean, pea, chickpea, soybean, carrot, strawberry, lettuce, oak, maple, walnut, rose, mint, squash, daisy, Quinoa, buckwheat, mung bean, cow pea, lentil, lupin, peanut, fava bean, French beans (i.e., common beans), mustard, or cactus. In some embodiments, the recombinant casein protein may be expressed in a non-vascular plant selected from moss, liverwort, homwort or algae. In some embodiments, the recombinant casein protein may be expressed in a vascular plant reproducing from spores (e.g., a fern). In some embodiments, the recombinant casein protein is expressed in a soybean plant.

In some embodiments, the recombinant casein protein is expressed in a microorganism. Microorganisms used for recombinant protein production are well known in the art (see for example, Ferrer-Miralles et al., Bacterial cell factories for recombinant protein production; expanding the catalogue, 2013, Microb Cell Fact. 2013; 12:113). In some embodiments, the recombinant casein protein is expressed in a yeast or a bacterium (i.e., it is “yeast-expressed” or “bacterial-expressed”). For example, the recombinant casein protein may be expressed in bacteria such as Escherichia coli, Caulobacter crescentus, Rodhobacter sphaeroides, Pseudoalteromonas haloplanktis, Shewanella sp., Pseudomonas putida, P. aeruginosa, P. fluorescens, Halomonas elongate, Chromohalobacter salexigens, Streptomyces lividans, S. griseus, Nocardia lactamdurans, Mycobacterium smegmatis, Corynebacterium glutamicum, C. ammoniagenes, Brevibacterium lactofermentum, Bacillus subtilis, B. brevis, B. megaterium, B. licheniformis, B. amyloliquefaciens, Lactococcus lactis, L. plantarum, L. casei, L. reuteri, or L. gasseri.

In some embodiments, the recombinant casein protein is expressed in a eukaryotic microorganism, such as Saccharomyces spp., Kluyveromyces spp., Pichia spp., Aspergillus spp., Tetrahymena spp., Yarrowla spp., Hansenula spp., Blastobotrys spp., Candida spp., Zygosaccharomyces spp., Debrayomyces spp., Fusarium spp., and Trichoderma spp.

In some embodiments, the solid-phase, protein stabilized emulsions comprise ash. In some embodiments, the solid-phase, protein stabilized emulsions comprise at least one lipid and at least one salt. “Lipid” means any of a class of molecules that are soluble in nonpolar solvents (such as ether and hexane) and relatively or completely insoluble in water. Lipid molecules are typically composed of long hydrocarbon tails that are hydrophobic in nature. Examples of lipids include fatty acids (saturated and unsaturated); glycerides or glycerolipids (such as monoglycerides, diglycerides, triglycerides or neutral fats, and phosphoglycerides or glycerophospholipids); and nonglycerides (sphingolipids, tocopherols, tocotrienols, sterol lipids including cholesterol and steroid hormones, prenol lipids including terpenoids, fatty alcohols, waxes, and polyketides).

Examples of lipids that may be included in the solid-phase, protein stabilized emulsion include, for example, dairy fats or vegetable oils such as palm oil or palm kernel oil, butter oil, anhydrous milkfat, soybean oil, corn oil, rapeseed oil, canola oil, sunflower oil, safflower oil, coconut oil, rice bran oil, olive oil, sesame oil, flaxseed oil, hemp oil, cottonseed oil, peanut oil, almond oil, beech nut oil, brazil nut oil, cashew oil, hazelnut oil, macadamia oil, mongongo nut oil, pecan oil, pine nut oil, pistachio oil, walnut oil, pumpkin seed oil, grapefruit seed oil, lemon oil, apricot oil, apple seed oil, argan oil, avocado oil, or orange oil. In some embodiments, the solid-phase, protein stabilized emulsion comprises butter or margarine.

Examples of salts that may be included in the emulsion include, but are not limited to, magnesium chloride, sodium chloride, calcium chloride, sodium phosphates and trisodium citrate.

In some embodiments, the emulsion comprises at least two plant-expressed casein proteins each selected from kappa-casein, para-kappa-casein, beta-casein, alpha-S1-casein and alpha-S2-casein. In some embodiments, the emulsion comprises at least three plant-expressed casein proteins each selected from kappa-casein, para-kappa-casein, beta-casein, alpha-S1-casein and alpha-S2-casein. In some embodiments, the emulsion comprises at least four plant-expressed casein proteins each selected from kappa-casein, para-kappa-casein, beta-casein, alpha-S1-casein and alpha-S2-casein. In some embodiments, the emulsion comprises at least one additional mammalian or plant protein that is not a casein protein.

Examples of combinations of casein, mammalian, and/or plant proteins that may be used in the solid phase, protein stabilized emulsions are shown below in Table 12. The casein or casein protein combination shown in Column 1 may be combined with one or more of the mammalian proteins listed in Column 2, and/or one or more of the plant proteins listed in Column three. In some embodiments, the solid-phase protein stabilized emulsions described herein comprise proteins from Column 1, and do not include any proteins from Column 2 or Column 3.

TABLE 12
Example combinations of casein, mammalian, and/or plant proteins

| | Mammalian proteins | Plant proteins |
|---|---|---|
| Casein proteins (Column 1) | (Column 2) | (Column 3) |
| κ-casein | Alpha-lactalbumin | Oleosins |
| Para-κ-casein | Beta-lactoglobulin | Leghemoglobin |
| β-casein | Albumin | Extensin-like protein |
| α-S1-casein | Lysozyme | family |
| α-S2-casein | Collagen family | Prolamine |
| κ-casein & para-κ-casein | Hemoglobin | Glutenin |
| κ-casein & β-casein | | Gamma-kafirin |
| κ-casein & α-S1-casein | | preprotein |
| κ-casein & α-S2-casein | | Alpha globulin |
| Para-κ-casein & β-casein | | Basic 7S globulin |
| Para-κ-casein & α-S1-casein | | precursor |
| Para-κ-casein & α-S2-casein | | 2S albumin |
| β-casein & α-S1-casein | | Beta-conglycinins |
| β-casein & α-S2-casein | | Glycinins |
| α-S1-casein & α-S2-casein | | Canein |
| κ-casein, para-κ-casein, & β-casein | | Zein |
| κ-casein, para-κ-casein, & α-S1-casein | | Patatin |
| κ-casein, para-κ-casein, & α-S2-casein | | Kunitz-Trypsin |
| Para-κ-casein, β-casein, & α-S1-casein | | inhibitor |
| Para-κ-casein, β-casein, & α-S2-casein | | Bowman-Birk |
| β-casein, α-S1-casein, & α-S2-casein | | inhibitor |
| κ-casein, β-casein, & α-S1-casein | | Cystatine |
| κ-casein, β-casein, & α-S2-casein | | |
| κ-casein, α-S1-casein & α-S2-casein | | |
| para-κ-casein, α-S1-casein & α-S2-casein | | |
| κ-casein, para-κ-casein, β-casein, α-S1- | | |
| casein | | |
| κ-casein, para-κ-casein, β-casein, & α-S2- | | |
| casein | | |
| Para-κ-casein, β-casein, α-S1-casein, & α- | | |
| S2-casein | | |
| κ-casein, β-casein, α-S1-casein, & α-S2- | | |
| casein | | |
| κ-casein, para-κ-casein, α-S1-casein & α- | | |
| S2-casein | | |

In some embodiments, the emulsion further comprises plant protein. For example, in some embodiments, the emulsion comprises protein from a legume, such as, for example, soybeans, chickpeas, kidney beans, black beans, pinto beans, green peas, and lentils. In some embodiments, the emulsion comprises protein from a grain, such as, for example, wheat, millet, barley, oats, rice, spelt, teff, amaranth, and quinoa. In some embodiments, the emulsion comprises protein from nuts, hempseed, chia seed, nutritional yeast, or spirulina. In some embodiment, the emulsion further comprises protein from potato. In some embodiments, the emulsion further comprises protein from a plant of the family Fabaceae.

In some embodiments, the emulsion has a pH of about 5.0 to about 6.7. In some embodiments, the emulsion has a pH of about 5.2 to about 5.9. In some embodiments, the emulsion has a pH of about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, or about 6.9.

In some embodiments, the emulsion may further comprise one or more additional agents, such as an edible gum, starch, and/or gelling agent. Examples of edible gums include, but are not limited to, curdian, locust bean gum, carrageenan, gellan gum, xanthan gum, guar gum, agar agar, gelatin, sodium alginate, or combinations thereof. Examples of starch include, but are not limited to, potato starch, corn starch, rice flour, pea flour, modified starch, and combinations thereof. Examples of gelling agents include, but are not limited to, pectin, alginate, vegetable gums, gelatin, agar, methyl cellulose, enzymes (transglutaminase) and hydoroxypropylmethyl cellulose. In some embodiments, the emulsion may further comprise an acid or abase, such as lemon juice, lactic acid, acetic acid, citric acid, sodium citrate, sodium orthophosphates, sodium pyrophosphates, sodium polyphosphates, potassium citrate, potassium orthophosphates, potassium pyrophosphates, sorbic acid, potassium sorbate, tartaric acid, and sodium aluminum phosphate.

In some embodiments, the emulsion does not contain an organoleptically functional amount of beta-lactoglobulin. In some embodiments, the emulsion may comprise beta-lactoglobulin in the amount of about 0.01% (w/v) to about 0.1% (w/v), about 0.1% (w/v) to about 0.5% (w/v), about 0.5% (w/v) to about 1.0% (w/v), about 1.0% (w/v) to about 2% (w/v), about 2% (w/v) to about 3% (w/v), about 3% (w/v) to about 5% (w/v), about 5% (w/v) to about 10% (w/v), about 10% (w/v) to about 20% (w/v), about 20% (w/v) to about 40% (w/v), or more, of the emulsion.

As used herein, an “organoleptically functional amount of beta-lactoglobulin” refers to an amount of beta-lactoglobulin that significantly impacts one or more organoleptic properties of the composition. An organoleptic property is “significantly impacted” if it represents a change that can be detected by a human, using one or more of the senses taste, sight, smell, and/or touch. In some embodiments, a solid-phase, protein stabilized emulsion that does not comprise an organoleptically functional amount of beta-lactoglobulin may comprise only trace amounts of beta-lactoglobulin. In some embodiments, the emulsion may comprise beta-lactoglobulin in the range of about 0.01% (w/v) to about 0.1% (w/v), about 0.1% (w/v) to about 0.5% (w/v), about 0.5% (w/v) to about 1.0% (w/v), about 1.0% (w/v) to about 2% (w/v), about 2% (w/v) to about 3% (w/v), about 3% (w/v) to about 5% (w/v), about 5% (w/v) to about 10% (w/v), about 10% (w/v) to about 20% (w/v), about 20% (w/v) to about 40% (w/v), or more, of the emulsion.

In some embodiments, a solid phase, protein-stabilized emulsion comprises one plant-expressed casein protein selected from kappa-casein, para-kappa-casein, beta-casein, alpha-S1-casein, and alpha-S2-casein; wherein the emulsion does not contain any additional casein proteins; and wherein the emulsion has at least one of the following characteristics: i) a firmness of at least 150 grams; ii) a melting point of about 35° C. to about 100° C.; or iii) ability to stretch to at least 3 cm in length without breaking. In some embodiments, the emulsion further comprises at least one lipid and at least one salt. In some embodiments, the plant-expressed casein protein is expressed in a soybean plant. In some embodiments, the emulsion has a pH of about 5.2 to about 5.9. In some embodiments, the emulsion does not contain an organoleptically functional amount of beta-lactoglobulin. In some embodiments, the emulsion may comprise beta-lactoglobulin in the amount of about 0.01% (w/v) to about 0.1% (w/v), about 0.1% (w/v) to about 0.5% (w/v), about 0.5% (w/v) to about 1.0% (w/v), about 1.0% (w/v) to about 2% (w/v), about 2% (w/v) to about 3% (w/v), about 3% (w/v) to about 5% (w/v), about 5% (w/v) to about 10% (w/v), about 10% (w/v) to about 20% (w/v), about 20% (w/v) to about 40% (w/v), or more.

In some embodiments, a solid phase, protein-stabilized emulsion comprises: a plant-expressed casein protein selected from kappa-casein, para-kappa-casein, beta-casein, alpha-S1-casein, and alpha-S2-casein; and further comprises plant-expressed beta-lactoglobulin; wherein the ratio of the casein protein to the beta-lactoglobulin is about 8:1 to about 1:2. In some embodiments, the emulsion has at least one of the following characteristics: i) a firmness of at least 150 grams; ii) a melting point of about 35° C. to about 100° C.; or iii) ability to stretch to at least 3 cm in length without breaking. In some embodiments, the emulsion comprises at least at least one additional mammalian or plant protein that is not a casein protein. In some embodiments, the ratio of the casein protein to the beta-lactoglobulin is 1:2. In some embodiments, the ratio of the casein protein to the beta-lactoglobulin is about 2:1. In some embodiments, the emulsion has a pH of about 5.2 to about 5.9.

In some embodiments, a solid-phase protein-stabilized emulsion comprises about 8% (w/v) to about 25% (w/v) total protein, such as about 8% to about 10%, about 10% to about 15%, about 15% to about 20%, or about 20 to about 25% total protein. In some embodiments, a solid-phase protein stabilized emulsion comprises about 1% to about 10% (w/v) total protein. In some embodiments, a solid-phase protein stabilized emulsion comprises about 25% to about 35%, about 35% to about 45%, about 45% to about 55%, about 55% to about 65%, about 65% to about 75% (w/v), or more total protein.

In some embodiments, about 1% to about 5% of the total protein in the solid-phase protein stabilized emulsion is casein protein. In some embodiments, about 5% to about 10% of the total protein in the solid-phase protein stabilized emulsion is casein protein. In some embodiments, about 10% to about 20% of the total protein in the solid-phase protein stabilized emulsion is casein protein. In some embodiments, about 20% to about 30% of the total protein in the solid-phase protein stabilized emulsion is casein protein. In some embodiments, about 30% to about 40% of the total protein in the solid-phase protein stabilized emulsion is casein protein. In some embodiments, about 40% to about 50% of the total protein in the solid-phase protein stabilized emulsion is casein protein. In some embodiments, about 50% to about 60% of the total protein in the solid-phase protein stabilized emulsion is casein protein. In some embodiments, about 60% to about 70% of the total protein in the solid-phase protein stabilized emulsion is casein protein. In some embodiments, about 70% to about 80% of the total protein in the solid-phase protein stabilized emulsion is casein protein. In some embodiments, about 80% to about 90% of the total protein in the solid-phase protein stabilized emulsion is casein protein. In some embodiments, about 90% to about 100% of the total protein in the solid-phase protein stabilized emulsion is casein protein.

In some embodiments, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, or more of the total protein in the solid-phase protein stabilized emulsion is casein protein.

In some embodiments, about 20% to about 100% of the casein protein in the solid-phase protein-stabilized emulsion is kappa casein. For example, the emulsion may comprise about 20% to about 30%, about 30% to about 40%, about 40% to about 50%, about 50% to about 60%, about 60% to about 70%, about 70% to about 80%, about 80% to about 90%, or about 90% to about 100% kappa casein. In some embodiments, about 20% to about 30%, about 30% to about 40%, about 40% to about 50%, about 50% to about 60%, about 60% to about 70%, about 70% to about 80%, about 80% to about 90%, or about 90% to about 100% of the casein protein in the solid-phase protein-stabilized emulsion is kappa casein.

In some embodiments, about 20% to about 100% of the casein protein in the solid-phase protein-stabilized emulsion is para-kappa casein. For example, the emulsion may comprise about 20% to about 30%, about 30% to about 40%, about 40% to about 50%, about 50% to about 60%, about 60% to about 70%, about 70% to about 80%, about 80% to about 90%, or about 90% to about 100% para-kappa casein. In some embodiments, about 20% to about 30%, about 30% to about 40%, about 40% to about 50%, about 50% to about 60%, about 60% to about 70%, about 70% to about 80%, about 80% to about 90%, or about 90% to about 100% of the casein protein in the solid-phase protein-stabilized emulsion is para-kappa casein.

In some embodiments, about 20% to about 100% of the casein protein in the solid-phase protein-stabilized emulsion is beta casein. In some embodiments, about 50% to about 100% of the casein protein in the solid-phase protein-stabilized emulsion is beta casein. For example, the emulsion may comprise about 20% to about 30%, about 30% to about 40%, about 40% to about 50%, about 50% to about 60%, about 60% to about 70%, about 70% to about 80%, about 80% to about 90%, or about 90% to about 100% beta casein. In some embodiments, about 20% to about 30%, about 30% to about 40%, about 40% to about 50%, about 50% to about 60%, about 60% to about 70%, about 70% to about 80%, about 80% to about 90%, or about 90% to about 100% of the casein protein in the solid-phase protein-stabilized emulsion is beta casein.

In some embodiments, about 20% to about 100% of the casein protein in the solid-phase protein-stabilized emulsion is alpha-S1-casein. In some embodiments, about 50% to about 100% of the casein protein in the solid-phase protein-stabilized emulsion is alpha-S1-casein. For example, the emulsion may comprise about 20% to about 30%, about 30% to about 40%, about 40% to about 50%, about 50% to about 60%, about 60% to about 70%, about 70% to about 80%, about 80% to about 90%, or about 90% to about 100% alpha-S1-casein. In some embodiments, about 20% to about 30%, about 30% to about 40%, about 40% to about 50%, about 50% to about 60%, about 60% to about 70%, about 70% to about 80%, about 80% to about 90%, or about 90% to about 100% of the casein protein in the solid-phase protein-stabilized emulsion is alpha-S1-casein.

In some embodiments, about 20% to about 100% of the casein protein in the solid-phase protein-stabilized emulsion is alpha-S2-casein. In some embodiments, about 50% to about 100% of the casein protein in the solid-phase protein-stabilized emulsion is alpha-S2-casein. For example, the emulsion may comprise about 20% to about 30%, about 30% to about 40%, about 40% to about 50%, about 50% to about 60%, about 60% to about 70%, about 70% to about 80%, about 80% to about 90%, or about 90% to about 100% alpha-S2-casein. In some embodiments, about 20% to about 30%, about 30% to about 40%, about 40% to about 50%, about 50% to about 60%, about 60% to about 70%, about 70% to about 80%, about 80% to about 90%, or about 90% to about 100% of the casein protein in the solid-phase protein-stabilized emulsion is alpha-S2-casein.

In some embodiments, a solid-phase protein-stabilized emulsion comprises about 8% (w/v) to about 25% (w/v) total protein, one or more lipids, and one or more salts; wherein at least 4% of the total protein comprises casein proteins selected from kappa-casein, para-kappa-casein, beta-casein, alpha-S1-casein, and alpha-S2-casein; wherein the emulsion has at least one of the following characteristics: i) a firmness of at least 150 grams; ii) a melting point of about 35° C. to about 100° C.; or iii) ability to stretch to at least 3 cm in length without breaking. In some embodiments, at least 20% to 100% of the casein protein is kappa casein. In some embodiments, at least 20% to 100% of the casein protein is para-kappa casein. In some embodiments, at least 50% to 100% of the casein protein is beta-casein. In some embodiments, at least 50% to 100% of the casein protein is alpha-S1-casein. In some embodiments, at least 20% to 100% of the casein protein is alpha-S2-casein. In some embodiments, casein protein is expressed in a plant. In some embodiments, the emulsion has a pH of about 5.2 to about 5.9. In some embodiments, the composition comprises only one, only two, only three, or only four casein proteins selected from kappa-casein, para-kappa-casein, beta-casein, alpha-S1-casein, and alpha-S2-casein. In some embodiments, the emulsion does not contain an organoleptically functional amount of beta-lactoglobulin. In some embodiments, the emulsion may comprise beta-lactoglobulin in the amount of about 0.01% (w/v) to about 0.1% (w/v), about 0.1% (w/v) to about 0.5% (w/v), about 0.5% (w/v) to about 1.0% (w/v), about 1.0% (w/v) to about 2% (w/v), about 2% (w/v) to about 3% (w/v), about 3% (w/v) to about 5% (w/v), about 5% (w/v) to about 10% (w/v), about 10% (w/v) to about 20% (w/v), about 20% (w/v) to about 40% (w/v), or more.

### Alternative Dairy Compositions Comprising One or More Isolated or Recombinant Casein Proteins

The milk or casein proteins described herein may also be used to prepare alternative dairy compositions. For example, in some embodiments, an alternative dairy composition comprises one or more casein proteins, such as recombinant casein proteins. In some embodiments, the casein proteins are selected from kappa-casein, para-kappa-casein, beta-casein, alpha-S1-casein and alpha-S2-casein. In some embodiments, the alternative dairy composition comprises only one casein protein. In some embodiments, the alterative diary composition comprises two, three, or four casein proteins.

In some embodiments, the disclosure relates to an alternative dairy composition comprising a casein protein selected from kappa-casein, para-kappa-casein, beta-casein, alpha-S1-casein, and alpha-S2-casein; and a beta-lactoglobulin. In some embodiments the casein protein is recombinant. In some embodiments, the beta-lactoglobulin is recombinant. In some embodiments, both the casein protein and the beta-lactoglobulin are recombinant. In some embodiments, the ratio of the casein protein to the beta-lactoglobulin is about 8:1 to about 1:2. In some embodiments, the ratio of the casein protein to the beta-lactoglobulin is about 8:1 to about 2:1.

In some embodiments, an alternative dairy composition comprises about 8% (w/v) to about 25% (w/v) total protein, such as about 8% to about 10%, about 10% to about 15%, about 15% to about 20%, or about 20 to about 25% total protein. In some embodiments, an alternative dairy composition comprises about 1% to about 10% (w/v) total protein. In some embodiments, an alternative dairy composition comprises about 25% to about 35%, about 35% to about 45%, about 45% to about 55%, about 55% to about 65%, about 65% to about 75% (w/v), or more total protein.

In some embodiments, about 1% to about 5% of the total protein in the alternative dairy composition is casein protein. In some embodiments, about 5% to about 10% of the total protein in the alternative dairy composition is casein protein. In some embodiments, about 10% to about 20% of the total protein in the alternative dairy composition is casein protein. In some embodiments, about 20% to about 30% of the total protein in the alternative dairy composition is casein protein. In some embodiments, about 30% to about 40% of the total protein in the alternative dairy composition is casein protein. In some embodiments, about 40% to about 50% of the total protein in the alternative dairy composition is casein protein. In some embodiments, about 50% to about 60% of the total protein in the alternative dairy composition is casein protein. In some embodiments, about 60% to about 70% of the total protein in the alternative dairy composition is casein protein. In some embodiments, about 70% to about 80% of the total protein in the alternative dairy composition is casein protein. In some embodiments, about 80% to about 90% of the total protein in the alternative dairy composition is casein protein. In some embodiments, about 90% to about 100% of the total protein in the alternative dairy composition is casein protein.

In some embodiments, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, or more of the total protein in the alternative dairy composition is casein protein.

In some embodiments, about 20% to about 100% of the casein protein in the alternative dairy composition is kappa casein. For example, the alternative dairy composition may comprise about 20% to about 30%, about 30% to about 40%, about 40% to about 50%, about 50% to about 60%, about 60% to about 70%, about 70% to about 80%, about 80% to about 90%, or about 90% to about 100% kappa casein. In some embodiments, about 20% to about 30%, about 30% to about 40%, about 40% to about 50%, about 50% to about 60%, about 60% to about 70%, about 70% to about 80%, about 80% to about 90%, or about 90% to about 100% of the casein protein in the alternative dairy composition is kappa casein.

In some embodiments, about 20% to about 100% of the casein protein in the alternative dairy composition is para-kappa casein. For example, the alternative dairy composition may comprise about 20% to about 30%, about 30% to about 40%, about 40% to about 50%, about 50% to about 60%, about 60% to about 70%, about 70% to about 80%, about 80% to about 90%, or about 90% to about 100% para-kappa casein. In some embodiments, about 20% to about 30%, about 30% to about 40%, about 40% to about 50%, about 50% to about 60%, about 60% to about 70%, about 70% to about 80%, about 80% to about 90%, or about 90% to about 100% of the casein protein in the alternative dairy composition is para-kappa casein.

In some embodiments, about 20% to about 100% of the casein protein in the alternative dairy composition is beta casein. In some embodiments, about 50% to about 100% of the casein protein in the alternative dairy composition is beta casein. For example, the alternative dairy composition may comprise about 20% to about 30%, about 30% to about 40%, about 40% to about 50%, about 50% to about 60%, about 60% to about 70%, about 70% to about 80%, about 80% to about 90%, or about 90% to about 100% beta casein. In some embodiments, about 20% to about 30%, about 30% to about 40%, about 40% to about 50%, about 50% to about 60%, about 60% to about 70%, about 70% to about 80%, about 80% to about 90%, or about 90% to about 100% of the casein protein in the alternative dairy composition is beta casein.

In some embodiments, about 20% to about 100% of the casein protein in the alternative dairy composition is alpha-S1-casein. In some embodiments, about 50% to about 100% of the casein protein in the alternative dairy composition is alpha-S1-casein. For example, the alternative dairy composition may comprise about 20% to about 30%, about 30% to about 40%, about 40% to about 50%, about 50% to about 60%, about 60% to about 70%, about 70% to about 80%, about 80% to about 90%, or about 90% to about 100% alpha-S1-casein. In some embodiments, about 20% to about 30%, about 30% to about 40%, about 40% to about 50%, about 50% to about 60%, about 60% to about 70%, about 70% to about 80%, about 80% to about 90%, or about 90% to about 100% of the casein protein in the alternative dairy composition is alpha-S1-casein.

In some embodiments, about 20% to about 100% of the casein protein in the alternative dairy composition is alpha-S2-casein. In some embodiments, about 50% to about 100% of the casein protein in the alternative dairy composition is alpha-S2-casein. For example, the alternative dairy composition may comprise about 20% to about 30%, about 30% to about 40%, about 40% to about 50%, about 50% to about 60%, about 60% to about 70%, about 70% to about 80%, about 80% to about 90%, or about 90% to about 100% alpha-S2-casein. In some embodiments, about 20% to about 30%, about 30% to about 40%, about 40% to about 50%, about 50% to about 60%, about 60% to about 70%, about 70% to about 80%, about 80% to about 90%, or about 90% to about 100% of the casein protein in the alternative dairy composition is alpha-S2-casein.

In some embodiments, an alternative dairy composition comprises kappa casein and essentially no para-kappa casein. For example, in some embodiments, the alternative dairy composition comprises less than about 1%, less than about 0.9%, less than about 0.8%, less than about 0.7%, less than about 0.6%, less than about 0.5%, less than about 0.4%, less than about 0.3%, less than about 0.2%, or less than about 0.1%, para-kappa casein. In some embodiments, the alternative dairy composition comprises about 0.01% to about 1%, about 0.010% to about 0.9%, about 0.010% to about 0.8%, about 0.010% to about 0.7%, about 0.010% to about 0.6%, about 0.1% to about 0.5%, about 0.1% to about 0.4%, about 0.1% to about 0.3%, about 0.1% to about 0.2%, or about 0.01% to about 0.1% para-kappa casein. In some embodiments, the kappa casein is recombinant. In some embodiments, the kappa casein is expressed in a plant. In some embodiments, the kappa casein is expressed in a soybean plant.

In some embodiments, an alternative dairy composition comprises one to four recombinant milk proteins, each selected from kappa-casein, para-kappa-casein, beta-casein, alpha-S1-casein, and alpha-S2-casein. In some embodiments, an alternative dairy composition comprises 1, 2, 3, or 4 casein proteins. In some embodiments, an alternative dairy composition comprises only one casein protein.

In some embodiments, an alternative dairy composition comprises recombinant beta-casein and at least one lipid and does not comprise an organoleptically functional amount of beta-lactoglobulin. In some embodiments, the composition does not comprise any additional casein proteins. In some embodiments, the composition comprises at least one additional casein protein. In some embodiments, the at least one additional casein protein is selected from kappa-casein, para-kappa-casein, alpha-S1-casein and alpha-S2-casein. In some embodiments, the at least one additional casein is kappa-casein or para-kappa-casein. In some embodiments, at least 50%, at least 75%, or at least 90% by weight of the total casein protein in an alternative dairy composition is beta-casein. In some embodiments, the beta-casein is expressed in a plant. In some embodiments, the beta-casein is expressed in a soybean plant. In some embodiments, all caseins in the composition are plant expressed. In some embodiments, the composition comprises a fusion protein comprising recombinant beta-casein.

In some embodiments, the alternative dairy composition comprises two of the milk proteins selected from kappa-casein, para-kappa-casein, beta-casein, alpha-S1-casein, and alpha-S2-casein. In some embodiments, the alternative dairy composition comprises three of the milk proteins selected from kappa-casein, para-kappa-casein, beta-casein, alpha-S1-casein, and alpha-S2-casein. In some embodiments, the alternative dairy composition comprises four of the milk proteins selected from kappa-casein, para-kappa-casein, beta-casein, alpha-S1-casein, and alpha-S2-casein. In some embodiments, the one or more milk protein(s) is(are) plant-expressed. In some embodiments, the milk protein(s) is(are) expressed in a soybean plant. In some embodiments, the milk protein(s) is(are) yeast- or bacterial-expressed. Exemplary combinations of 1, 2, 3, or 4 casein proteins that may be used in the alternative dairy compositions described herein are shown above in Table 12.

In some embodiments, the disclosure relates to an alternative dairy composition comprising one to four plant-expressed recombinant milk proteins (i.e., 2, 3, or 4 plant-expressed recombinant milk proteins), wherein the recombinant milk proteins confer one, two, three or more organoleptic properties similar to a dairy composition (i.e., a dairy composition comprising mammalian milk such as bovine milk) selected from the group consisting of taste, appearance, mouthfeel, structure, texture, density, elasticity, springiness, coagulation, binding, leavening, aeration, foaming, creaminess, and emulsification. In some embodiments, the plant-expressed milk proteins are selected from beta lactoglobulin, kappa-casein, para-kappa-casein, beta-casein, alpha-S1-casein, and alpha-S2-casein. In some embodiments, the recombinant beta-casein protein confers on the alternative dairy composition one, two, or more characteristics of a dairy food product selected from the group consisting of: taste, aroma, appearance, handling, mouthfeel, density, structure, texture, elasticity, springiness, coagulation, binding, leavening, aeration, foaming, creaminess and emulsification.

In some embodiments, the alternative dairy compositions described above comprise at least one additional mammalian or plant protein that is not a casein protein. Examples of combinations of casein, mammalian, and/or plant proteins are shown above in Table 12.

In some embodiments, the alternative dairy compositions described herein may comprise plant protein. For example, in some embodiments, the alternative dairy compositions comprise protein from a legume, such as, for example, soybeans, chickpeas, kidney beans, black beans, pinot beans, green peas, and lentils. In some embodiments, the alternative dairy compositions comprise protein from a grain, such as, for example, wheat, millet, barley, oats, rice, spelt, teff, amaranth, and quinoa. In some embodiments, the alternative dairy compositions comprise protein from nuts, hempseed, chia seed, nutritional yeast, or spirulina. In some embodiments, the alternative diary composition comprises protein from potato. In some embodiments, the alternative diary composition comprises protein from a plant of the family Fabaceae.

In some embodiments, the alternative dairy compositions described above have at least one of the following characteristics: i) a firmness of at least 150 grams; ii) a melting point of about 35° C. to about 100° C.; or iii) ability to stretch to at least 3 cm in length without breaking. In some embodiments, the alternative diary compositions described above have the ability to stretch to at least 4 cm, at least 5 cm, at least 6 cm, at least 7 cm, at least 8 cm, at least 9 cm, at least 10 cm, at least 11 cm, at least 12 cm, at least 13 cm, at least 14 cm, at least 15 cm, at least 16 cm, at least 17 cm, at least 18 cm, at least 19 cm, or at least 10 cm in length without breaking. In some embodiments, the alternative dairy compositions described above have the ability to stretch to at least 5 cm in length without breaking. Testing methods and ranges firmness, melting point, and stretch are disclosed above.

In some embodiments, the alternative diary compositions comprise ash. In some embodiments, the alternative dairy compositions comprise at least one lipid and/or at least one salt. Examples of lipids include fatty acids (saturated and unsaturated); glycerides or glycerolipids (such as monoglycerides, diglycerides, triglycerides or neutral fats, and phosphoglycerides or glycerophospholipids); and nonglycerides (sphingolipids, tocopherols, tocotrienols, sterol lipids including cholesterol and steroid hormones, prenol lipids including terpenoids, fatty alcohols, waxes, and polyketides).

Examples of lipids that may be included in the alternative dairy compositions include, for example, dairy fats or vegetable oils such as palm oil or palm kernel oil, soybean oil, corn oil, rapeseed oil, canola oil, sunflower oil, safflower oil, coconut oil, rice bran oil, olive oil, sesame oil, flaxseed oil, hemp oil, cottonseed oil, peanut oil, almond oil, beech nut oil, brazil nut oil, cashew oil, hazelnut oil, macadamia oil, mongongo nut oil, pecan oil, pine nut oil, pistachio oil, walnut oil, pumpkin seed oil, grapefruit seed oil, lemon oil, apricot oil, apple seed oil, argan oil, avocado oil, or orange oil. In some embodiments, the solid-phase, protein stabilized emulsion comprises butter or margarine.

Examples of salts that may be included in the alternative dairy composition include, but are not limited to, magnesium chloride, sodium chloride, calcium chloride, sodium phosphate and trisodium citrate.

In some embodiments, the alternative dairy compositions do not contain an organoleptically functional amount of beta-lactoglobulin. In some embodiments, the alternative dairy composition may comprise beta-lactoglobulin in the amount of about 0.01% (w/v) to about 0.1% (w/v), about 0.1% (w/v) to about 0.5% (w/v), about 0.5% (w/v) to about 1.0% (w/v), about 1.0% (w/v) to about 2% (w/v), about 2% (w/v) to about 3% (w/v), about 3% (w/v) to about 5% (w/v), about 5% (w/v) to about 10% (w/v), about 10% (w/v) to about 20% (w/v), about 30% (w/v) to about 40% (w/v), or more, of the composition.

In some embodiments, the alternative dairy compositions comprise one or more recombinant casein proteins that are expressed in a microorganism. In some embodiments, the recombinant casein protein is yeast-expressed or bacterial-expressed. In some embodiments, the recombinant casein protein is expressed in a bacterium. Microorganisms used for recombinant protein production are well known in the art (see for example, Ferrer-Miralles et al., Bacterial cell factories for recombinant protein production; expanding the catalogue, 2013, Microb Cell Fact. 2013; 12:113). For example, the recombinant casein protein may be expressed in a bacteria such as Escherichia coli, Caulobacter crescentus, Rodhobacter sphaeroides, Pseudoalteromonas haloplanktis, Shewanella sp., Pseudomonas putida, P. aeruginosa, P. fluorescens, Halomonas elongate, Chromohalobacter salexigens, Streptomyces lividans, S. griseus, Nocardia lactamdurans, Mycobacterium smegmatis, Corynebacterium glutamicum, C. ammoniagenes, Brevibacterium lactofermentum, Bacillus subtilis, B. brevis, B. megaterium, B. licheniformis, B. amyloliquefaciens, Lactococcus lactis, L. plantarum, L. casei, L. reuteri, or L. gasseri.

In some embodiments, the recombinant casein proteins are expressed in a microorganism that is a eukaryotic cell, such as Saccharomyces spp., Kluyveromyces spp., Pichia spp., Aspergillus spp., Tetrahymena spp., Yarrowla spp., Hansenula spp., Blastobotrys spp., Candida spp., Zygosaccharomyces spp., Debrayomyces spp., Fusarium spp., and Trichoderma spp.

In some embodiments, the one or more recombinant casein proteins are expressed in a plant. In some embodiments, the plant may be a monocot selected from turf grass, maize (corn), rice, oat, wheat, barley, sorghum, orchid, iris, lily, onion, palm, and duckweed. In some embodiments, the plant is a dicot selected from Arabidopsis, tobacco, tomato, potato, sweet potato, cassava, alfalfa, lima bean, pea, chickpea, soybean, carrot, strawberry, lettuce, oak, maple, walnut, rose, mint, squash, daisy, Quinoa, buckwheat, mung bean, cow pea, lentil, lupin, peanut, fava bean, French beans (i.e., common beans), mustard, or cactus. In some embodiments, the plant is a non-vascular plant selected from moss, liverwort, hornwort or algae. In some embodiments, the plant is a vascular plant reproducing from spores (e.g., a fern). In some embodiments, the recombinant casein protein is expressed in a soybean plant.

In some embodiments, the alternative dairy compositions described above have a pH of about 2 to about 8. In some embodiments, the alternative dairy compositions described above have a pH of about 4 to about 8. Table 13 below shows exemplary ranges of pH for common mammalian derived dairy products.

TABLE 13
pH ranges of common dairy products

| Dairy product | pH range |
|---|---|
| Milk | 6.7-6.9 |
| Butter | 6.1-6.4 |
| Yogurt | 2.0-4.5 |
| Brie | 6.0-6.5 |
| Cheddar | 5.1-5.3 |
| Cream cheese | 4.6-5.1 |
| Feta | 4.1-4.5 |
| Parmesan | 5.2-5.3 |
| Ricotta | 6.0 |

Examples of alternative dairy compositions that may be produced as described herein include, but are not limited to, alternative versions of milk, cream, butter, and cheese. Other example alternative dairy compositions include ice cream, frozen desserts, frozen yogurt or custard, yogurt, cottage cheese, cream cheese, curds, crème fraiche, toppings, icings, fillings, low-fat spreads, dairy-based dry mixes, geriatric nutrition compositions, coffee creamers, analog dairy products, follow-up formula, baby formula, infant formula, milk, dairy beverages, acid dairy drinks, smoothies, milk tea, margarine, butter alternatives, growing up milks, low-lactose products, buttermilk, sour cream, skyr, leben, lassi, kefir, and beverages. In some embodiments, the alternative diary compositions may be cultured milks, such as drinkable yogurts. The alternative dairy compositions may also be powders containing a milk protein, or a low-lactose product. An illustrative method for preparing an alternative dairy composition is provided in FIG. 13.

An alternative milk composition may be produced, for example, by mixing a liquid comprising at least one isolated or recombinant milk or casein protein, with ash, lipids, and/or a sweetener, and optionally one or more flavor compounds and/or color agents. In some embodiments, one or more vitamins are added to the alternative milk composition, such as retinal, carotene, vitamins, vitamin D, vitamin E, vitamin B12, thiamin, or riboflavin. This milk alternative may then be used to produce, for example, butter, ice cream, frozen desserts, frozen yogurt or custard, yogurt, cottage cheese, cream cheese, curds, tofu, and crème fraiche.

In some embodiments, the alternative dairy composition comprises one or more sweeteners. Examples of sweeteners include, but are not limited to, saccharides, such as glucose, mamiose, maltose, fructose, galactose, lactose, sucrose, monatin, and tagatose. In some embodiments the sweetener is selected from stevia, aspartame, cyclamate, saccharin, sucralose, mogrosides, brazzein, curculin, erythritol, glycyrrhizin, inulin, isomalt, lacititol, mabinlin, malititol, mamiitol, miraculin, monatin, monelin, osladin, pentadin, sorbitol, thaumatin, xylitol, acesulfame, potassium, advantame, alitame, aspartame-acesulfame, sodium cyclamate, dulcin, glucin, neohesperidin, dihyrdochalcone, neotame, and P-4000.

In some embodiments, an alternative dairy food composition comprises calcium. In some embodiments, the composition comprises calcium at a concentration of about 0% to about 2% by weight. In some embodiments, the composition comprises calcium at a concentration of about 0.001% to about 2% by weight. In some embodiments, the composition comprises calcium at a concentration of about 0.01% to about 2% by weight. In some embodiments, the composition comprises calcium at a concentration of about 0.1% to about 2% by weight. In some embodiments, the composition comprises calcium at a concentration of about 1% to about 2% by weight. In some embodiments, the composition comprises calcium at a concentration of about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, or about 2.0% by weight.

Thus, in some embodiments, the alternative dairy composition is a milk composition. In some embodiments, the alternative dairy composition is a cheese composition. In some embodiments, the alternative dairy composition is cream composition. In some embodiments, the alternative dairy composition is a yogurt composition (e.g., a frozen yogurt composition, a sugar-free yogurt composition, a low-fat yogurt composition, a Greek yogurt composition, a drinkable yogurt composition, etc.). In some embodiments, the alternative dairy composition is ice cream. In some embodiments, alternative dairy composition is a frozen custard composition. In some embodiments, the alternative dairy composition is a frozen dessert. In some embodiments, the alternative dairy composition is a crème fraiche composition. In some embodiments, the alternative dairy composition is curd composition. In some embodiments, the alternative dairy composition is a cottage cheese composition. In some embodiments, the alternative dairy composition is cream composition. In some embodiments, the alternative dairy composition is a sour cream composition.

### Cheese Compositions

Traditionally, cheese is made with milk, which comprises a number of proteins including various casein proteins (see Table 14 below for exemplary compositions of human and cow milk). Coagulation of the milk proteins occurs by way of an acid and/or rennet addition, which causes the milk to curdle. Rennet is a bacterial enzyme that cleaves kappa-casein, generating para-kappa-casein, which then links up with the calcium and phosphate present in milk to join casein micelles together. These solids curds are collected and/or separated from the liquid (whey) and various procedures of pressing, forming, and aging yield different cheese products.

TABLE 14
Illustrative Milk Protein Compositions

| | Human milk | Bovine (cow) milk |
|---|---|---|
| Protein | (mg/mL) | (mg/mL) |
| α-lactalbumin | 2.2 | 1.2 |
| α-s1-casein | 0 | 11.6 |
| α-s2-casein | 0 | 3.0 |
| β-casein | 2.2 | 9.6 |
| κ-casein | 0.4 | 3.6 |
| γ-casein | 0 | 1.6 |
| Immunoglobulins | 0.8 | 0.6 |
| Lactoferrin | 1.4 | 0.3 |
| β-lactoglobulin | 0 | 3.0 |
| Lysozyme | 0.5 | Traces |
| Serum albumin | 0.4 | 0.4 |
| Other | 0.8 | 0.6 |

Described herein are cheese compositions comprising a different protein composition compared to that of any mammalian milk (i.e., a non-naturally occurring protein composition). For example, in some embodiments, a cheese composition can be prepared using only one milk protein. In some embodiments, a cheese composition can be prepared using only two milk proteins. In some embodiments, a cheese composition may be prepared using only three milk proteins. In some embodiments, a cheese composition may be prepared using only four milk proteins. In some embodiments, a cheese composition comprises one or more milk proteins at a ratio that is not found in any mammalian milk (e.g., a non-naturally occurring ratio).

In some embodiments, a cheese composition comprises one milk protein, which may be derived from animal-produced milk, or recombinantly expressed. In some embodiments, a cheese composition comprises two, three, our four milk proteins, wherein each milk protein is derived from animal-produced milk or is recombinantly expressed. In some embodiments, the milk protein is a casein protein.

In some embodiments, a cheese composition may comprise beta-casein as the only casein protein (i.e., 100% beta-casein). In some embodiments, a cheese composition comprises beta-casein and at least one additional casein protein. In some embodiments, the at least one additional casein protein is selected from kappa-casein, para-kappa-casein, beta-casein, alpha-S1-casein and alpha-S2-casein. In some embodiments, the at least one additional casein protein is kappa-casein. In some embodiments, the at least one additional casein protein is para-kappa-casein.

In some embodiments, a cheese composition comprises two or more casein proteins, wherein about 95% by weight of the casein protein in the composition is beta-casein. In some embodiments, a cheese composition comprises two or more casein proteins, wherein about 90% by weight of the casein protein in the composition is beta-casein. In some embodiments, a cheese composition comprises two or more casein proteins, wherein about 85% by weight of the casein protein in the composition is beta-casein. In some embodiments, a cheese composition comprises two or more casein proteins, wherein about 80% by weight of the casein protein in the composition is beta-casein. In some embodiments, a cheese composition comprises two or more casein proteins, wherein about 75% by weight of the casein protein in the composition is beta-casein. In some embodiments, a cheese composition comprises two or more casein proteins, wherein about 70% by weight of the casein protein in the composition is beta-casein. In some embodiments, a cheese composition comprises two or more casein proteins, wherein about 65% by weight of the casein protein in the composition is beta-casein. In some embodiments, a cheese composition comprises two or more casein proteins, wherein about 60% by weight of the casein protein in the composition is beta-casein. In some embodiments, a cheese composition comprises two or more casein proteins, wherein about 55% by weight of the casein protein in the composition is beta-casein. In some embodiments, a cheese composition comprises two or more casein proteins, wherein about 50% by weight of the casein protein in the composition is beta-casein.

In some embodiments, a cheese composition may comprise 95%, beta-casein and 5% of one or more additional casein proteins. In some embodiments, it may comprise 90%, beta-casein and 10% of one or more additional casein proteins. In some embodiments, it may comprise 85%, beta-casein and 15% of one or more additional casein proteins. In some embodiments, it may comprise 80%, beta-casein and 20% of one or more additional casein proteins. In some embodiments, it may comprise 75%, beta-casein and 25% of one or more additional casein proteins. In some embodiments, it may comprise 70%, beta-casein and 30% of one or more additional casein proteins. The other casein proteins may be kappa-casein, para-kappa-casein, alpha-S1-casein, and/or alpha-S2-casein.

In some embodiments, the cheese composition comprises 75% beta-casein and 25% alpha caseins (i.e., a mixture of alpha-S1-casein and alpha-S2-casein). In some embodiments, the cheese composition comprises 75% beta-casein and 25% kappa-casein. In some embodiments, the cheese composition comprises 50% beta-casein and 50% kappa-casein. In some embodiments, the cheese composition comprises 50% beta-casein and 50% alpha caseins.

In some embodiments the beta-casein is recombinant beta-casein. In some embodiments, the recombinant beta-casein protein is plant-expressed. In some embodiments, the recombinant beta-casein is expressed in a soybean. In some embodiments, all the caseins in the cheese composition are plant-expressed. In some embodiments, the recombinant casein protein is derived from a fusion protein. In some embodiments, the cheese composition does not contain an organoleptically functional amount of beta-lactoglobulin.

In some embodiments, a cheese composition comprises para-kappa-casein produced without the use of any enzyme that cleaves kappa-casein to para-kappa-casein. In some embodiments, a cheese composition comprises para-kappa-casein produced without the use of any acid that cleaves kappa-casein to para-kappa-casein. In some embodiments, a cheese composition comprises para-kappa-casein produced without the use of any enzyme or acid that cleaves kappa-casein to para-kappa-casein. In some embodiments, a cheese composition comprises a recombinantly expressed para-kappa-casein. In some embodiments, a cheese composition comprises substantially no casein, such as 0.01% (w/v) to 0.1% (w/v) or 0.1% (w/v) to 0.1% (w/v) casein.

In some embodiments, a cheese composition comprises about 8% (w/v) to about 25% (w/v) total protein, such as about 8% to about 10%, about 10% to about 15%, about 15% to about 20%, or about 20 to about 25% total protein. In some embodiments, a cheese composition comprises about 1% to about 10% (w/v) total protein. In some embodiments, a cheese composition comprises about 25% to about 35%, about 35% to about 45%, about 45% to about 55%, about 55% to about 65%, about 65% to about 75% (w/v), or more total protein.

In some embodiments, about 1% to about 5% of the total protein in the cheese composition is casein protein. In some embodiments, about 5% to about 10% of the total protein in the cheese composition is casein protein. In some embodiments, about 10% to about 20% of the total protein in the cheese composition is casein protein. In some embodiments, about 20% to about 30% of the total protein in the cheese composition is casein protein. In some embodiments, about 30% to about 40% of the total protein in the cheese composition is casein protein. In some embodiments, about 40% to about 50% of the total protein in the cheese composition is casein protein. In some embodiments, about 50% to about 60% of the total protein in the cheese composition is casein protein. In some embodiments, about 60% to about 70% of the total protein in the cheese composition is casein protein. In some embodiments, about 70% to about 80% of the total protein in the cheese composition is casein protein. In some embodiments, about 80% to about 90% of the total protein in the cheese composition is casein protein. In some embodiments, about 90% to about 100% of the total protein in the cheese composition is casein protein.

In some embodiments, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, or more of the total protein in the cheese composition is casein protein.

In some embodiments, about 20% to about 100% of the casein protein in the cheese composition is kappa casein. For example, the cheese composition may comprise about 20% to about 30%, about 30% to about 40%, about 40% to about 50%, about 50% to about 60%, about 60% to about 70%, about 70% to about 80%, about 80% to about 90%, or about 90% to about 100% kappa casein. In some embodiments, about 20% to about 30%, about 30% to about 40%, about 40% to about 50%, about 50% to about 60%, about 60% to about 70%, about 70% to about 80%, about 80% to about 90%, or about 90% to about 100% of the casein protein in the cheese composition is kappa casein.

In some embodiments, about 20% to about 100% of the casein protein in the cheese composition is para-kappa casein. For example, the cheese composition may comprise about 20% to about 30%, about 30% to about 40%, about 40% to about 50%, about 50% to about 60%, about 60% to about 70%, about 70% to about 80%, about 80% to about 90%, or about 90% to about 100% para-kappa casein. In some embodiments, about 20% to about 30%, about 30% to about 40%, about 40% to about 50%, about 50% to about 60%, about 60% to about 70%, about 70% to about 80%, about 80% to about 90%, or about 90% to about 100% of the casein protein in the cheese composition is para-kappa casein.

In some embodiments, about 20% to about 100% of the casein protein in the cheese composition is beta casein. In some embodiments, about 50% to about 100% of the casein protein in the cheese composition is beta casein. For example, the cheese composition may comprise about 20% to about 30%, about 30% to about 40%, about 40% to about 50%, about 50% to about 60%, about 60% to about 70%, about 70% to about 80%, about 80% to about 90%, or about 90% to about 100% beta casein. In some embodiments, about 20% to about 30%, about 30% to about 40%, about 40% to about 50%, about 50% to about 60%, about 60% to about 70%, about 70% to about 80%, about 80% to about 90%, or about 90% to about 100% of the casein protein in the cheese composition is beta casein.

In some embodiments, about 20% to about 100% of the casein protein in the cheese composition is alpha-S1-casein. In some embodiments, about 50% to about 100% of the casein protein in the cheese composition is alpha-S1-casein. For example, the cheese composition may comprise about 20% to about 30%, about 30% to about 40%, about 40% to about 50%, about 50% to about 60%, about 60% to about 70%, about 70% to about 80%, about 80% to about 90%, or about 90% to about 100% alpha-S1-casein. In some embodiments, about 20% to about 30%, about 30% to about 40%, about 40% to about 50%, about 50% to about 60%, about 60% to about 70%, about 70% to about 80%, about 80% to about 90%, or about 90% to about 100% of the casein protein in the cheese composition is alpha-S1-casein.

In some embodiments, about 20% to about 100% of the casein protein in the cheese composition is alpha-S2-casein. In some embodiments, about 50% to about 100% of the casein protein in the cheese composition is alpha-S2-casein. For example, the cheese composition may comprise about 20% to about 30%, about 30% to about 40%, about 40% to about 50%, about 50% to about 60%, about 60% to about 70%, about 70% to about 80%, about 80% to about 90%, or about 90% to about 100% alpha-S2-casein. In some embodiments, about 20% to about 30%, about 30% to about 40%, about 40% to about 50%, about 50% to about 60%, about 60% to about 70%, about 70% to about 80%, about 80% to about 90%, or about 90% to about 100% of the casein protein in the cheese composition is alpha-S2-casein.

In some embodiments, a cheese composition comprises a stable, protein-stabilized emulsion described herein. In some embodiments, a cheese composition comprises more than one of the stable, protein-stabilized emulsions described herein. In some embodiments, a cheese composition is made using at least one stable, protein-stabilized emulsion described herein.

In some embodiments, a cheese composition comprises a colloidal suspension described herein. In some embodiments, a cheese composition comprises more than one colloidal suspension described herein. In some embodiments, a cheese composition is made using at least one of the colloidal suspensions described herein.

In some embodiments, a cheese composition described herein may comprise plant protein. For example, in some embodiments, the cheese composition comprises protein from a legume, such as, for example, soybeans, chickpeas, kidney beans, black beans, pinto beans, green peas, and lentils. In some embodiments, the cheese composition comprises protein from a grain, such as, for example, wheat, millet, barley, oats, rice, spelt, teff, amaranth, and quinoa. In some embodiments, the cheese composition comprises protein from nuts, hempseed, chia seed, nutritional yeast, or spirulina. In some embodiments, the cheese composition comprises protein from potato. In some embodiments, the cheese composition comprises protein from a plant of the family Fabaceae.

In some embodiments, the cheese compositions described herein may be substantially transparent. As used herein, “substantially transparent” means having an opacity of about 50%, about 40%, about 30%, about 20%, about 10% or less. In some embodiments, the cheese composition has about 0% opacity. In some embodiments, the cheese compositions described herein are substantially transparent when in solid form. In some embodiments, the cheese compositions described herein are substantially transparent when melted.

In some embodiments, the cheese compositions described herein may have at least one, at least two, or at least three desirable organoleptic properties. In some embodiments, the cheese compositions described herein may have at least one, at least two, or at least three organoleptic properties that is similar to that of cheese (i.e., cheese produced using mammalian milk, such as bovine milk or goat milk). For example, in some embodiments, the cheese compositions may have at least one, at least two, or at least three organoleptic properties found in the cheeses of Table 15 or Table 16.

In some embodiments, the cheese compositions described herein may be used in a similar manner (e.g., for cooking, etc.) as one or more of the cheeses listed in Table 15 or Table 16. In some embodiments, the cheese compositions described herein may be used as a substitute for one or more of the cheeses listed in Table 15 or Table 16.

TABLE 15
Illustrative types of cheese

| Category | Examples |
|---|---|
| Soft Fresh Cheeses | Cottage Cheese |
| | Cream Cheese |
| | Feta |
| | Mascarpone |
| | Neufchâtel |
| | Queso Blanco |
| | Ricotta |
| Soft-Ripened Cheeses | Brie (single, double and triple cream and |
| | flavored) |
| | Camembert |
| Semi-Soft Chesses | Brick, dry- and washed-rind |
| | Fontina |
| | Havarti |
| | Limburger |
| | Monterey Jack |
| | Muenster |
| | Pepper Jack |
| Blue-Veined Cheeses | Blue Cheese |
| | Gorgonzola, creamy style |
| | Gorgonzola, crumbly style |
| Gouda & Edam | Gouda |
| | Smoked Gouda |
| | Edam |
| Pasta Filata and Related | Fresh Mozzarella |
| Cheeses | Low-Moisture, Part-Skim Mozzarella |
| | Low-Moisture, Whole Milk Mozzarella |
| | Part-Skim Mozzarella |
| | Whole Milk Mozzarella |
| | Provolone, mild, aged and smoked |
| | String Cheese |
| | Pizza Cheese |
| | Individually Quick Frozen mozzarella (IQF) |
| Cheddar & Colby | Cheddar |
| | Smoked Cheddar |
| | Colby |
| Swiss Cheeses | Baby Swiss |
| | Swiss |
| | Gruyère |
| Hard Cheeses | Asiago |
| | Parmesan |
| | Romano |
| | Pepato |
| Process Cheeses | Pasteurized Process Cheese |
| | Pasteurized Process Cheese Food |
| | Pasteurized Process Cheese Spread |
| | Pasteurized Process Cheese Product |
| | Cold-Pack |
| | High-Melt Cheeses |
| Powder & Enzyme- | Cheese Powders |
| modified Cheeses | Enzyme Modified Cheeses (EMCs) |
| Custom & Convenience | Pre-blends |
| Cheese Products | Pre-cut Cheese |
| | Shredded Cheese |
| | Grated Cheese |
| | Cheese Sauce |
| | Portion Packaged Cheese |
| Cheeses for Special Needs | Low-fat Cheeses |
| | No-fat Cheeses |
| | Low-sodium Cheeses |
| | Kosher Cheeses |
| | Halal Cheeses |
| | Organic Cheeses |

Cheese may also be categorized based on moisture content. Shown below in Table 16 are example categories of cheeses and their respective moisture content (from Jana A H et al., J. Food Sci Technol (2017) 54(12):3776-3778).

TABLE 16
Moisture content of cheeses

| | Moisture | |
|---|---|---|
| Cheese type | content (%) | Examples |
| Soft cheese | 50-80 | Cottage, Quark, Baker's, Mozzarella, |
| | | Camembert, Feta |
| Semi-soft cheese | 39-50 | Blue, Limburger, Provolone, Tilsiter |
| Hard cheese | Max. 39 | Cheddar, Colby, Edam, Swiss, Gouda |
| Very hard cheese | Max. 34 | Parmesan, Romano, Sardo, Grana |

In some embodiments, a cheese composition described herein has a moisture content of between about 30% and about 80%. In some embodiments, a cheese composition described herein has between about 45% to 60% moisture content.

Cheese and cheese compositions have functional properties such as moisture content, firmness, stretchability, melting, viscosity/flow, oiling off, browning/blistering, whitening/decolorization, spreadability, grating, slicing, dicing, shredding/mincing, mouthfeel, flavor, aroma, freezing ability, and overall appearance. These properties can be determined by any number of means well known in the art.

Firmness and stretch may be analyzed as described above. Moisture content may be measured for example, as described in Bradley, R. L., Jr., and M. A. Vanderwarn. 2001, Determination of moisture in cheese and cheese products, J. AOAC 84:570-592. Texture may be analyzed as described in Kapoor et al., 2005, Small-scale manufacture of process cheese using a rapid visco analyzer, J. Dairy Sci. 88:3382-3391, using a TA.XT2 Texture Analyzer (see also Drake et al., 1999 Relationship between instrumental and sensory measurements of cheese texture, J. Texture Stud. 30:451-476) or for example by Breene 1975, Application of texture profile analysis to instrumental food texture evaluation, J. Texture Stud. 6:53-82.

In some embodiments, a cheese composition has the ability to stretch to at least 3 cm in length without breaking, as determined by heating a 100 gram mass of the composition at a temperature of 225° C. for 4 minutes and cooling to about 90° C. and pulling with a fork placed beneath the mass. In some embodiments, a cheese composition has the ability to stretch to at least 4 cm in length without breaking, as determined by heating a 100 gram mass of the composition at a temperature of 225° C. for 4 minutes and cooling to about 90° C. and pulling with a fork placed beneath the mass. In some embodiments, a cheese composition has the ability to stretch to at least 5 cm in length without breaking, as determined by heating a 100 gram mass of the composition at a temperature of 225° C. for 4 minutes and cooling to about 90° C. and pulling with a fork placed beneath the mass. In some embodiments, a cheese composition has the ability to stretch to at least 6 cm in length without breaking, as determined by heating a 100 gram mass of the composition at a temperature of 225° C. for 4 minutes and cooling to about 90° C. and pulling with a fork placed beneath the mass. In some embodiments, a cheese composition has the ability to stretch to at least 9 cm in length without breaking, as determined by heating a 100 gram mass of the composition at a temperature of 225° C. for 4 minutes and cooling to about 90° C. and pulling with a fork placed beneath the mass. In some embodiments, a cheese composition has the ability to stretch to at least 12 cm in length without breaking, as determined by heating a 100 gram mass of the composition at a temperature of 225° C. for 4 minutes and cooling to about 90° C. and pulling with a fork placed beneath the mass. In some embodiments, a cheese composition has the ability to stretch to at least 15 cm in length without breaking, as determined by heating a 100 gram mass of the composition at a temperature of 225° C. for 4 minutes and cooling to about 90° C. and pulling with a fork placed beneath the mass. In some embodiments, a cheese composition has the ability to stretch to at least 18 cm in length without breaking, as determined by heating a 100 gram mass of the composition at a temperature of 225° C. for 4 minutes and cooling to about 90° C. and pulling with a fork placed beneath the mass.

In some embodiments, a cheese composition described herein has a firmness of at least 150 grams, as determined by compressing a cylindrical-shaped sample of the cheese composition having a height of 3 cm and a diameter of 3 cm to a height of 1.5 cm at 5° C. In some embodiments, a cheese composition described herein has a firmness of at least 300 grams, as determined by compressing a cylindrical-shaped sample of the cheese composition having a height of 3 cm and a diameter of 3 cm to a height of 1.5 cm at 5° C. In some embodiments, a cheese composition described herein has a firmness of at least 600 grams, as determined by compressing a cylindrical-shaped sample of the cheese composition having a height of 3 cm and a diameter of 3 cm to a height of 1.5 cm at 5° C. In some embodiments, a cheese composition described herein has a firmness of at least 1000 grams, as determined by compressing a cylindrical-shaped sample of the cheese composition having a height of 3 cm and a diameter of 3 cm to a height of 1.5 cm at 5° C. In some embodiments, a cheese composition described herein has a firmness of at least 2000 grams, as determined by compressing a cylindrical-shaped sample of the cheese composition having a height of 3 cm and a diameter of 3 cm to a height of 1.5 cm at 5° C. In some embodiments, a cheese composition described herein has a firmness in the range of about 600 to about 3000 grams, for example about 650 to about 1000 grams, about 1000 grams to about 1500 grams, about 1500 grams to about 2000 grams, about 2500 grams to about 3000 grams, as determined by compressing a cylindrical-shaped sample of the cheese composition having a height of 3 cm and a diameter of 3 cm to a height of 1.5 cm at 5° C.

As will be understood by those of skill in the art, melting properties can be influenced by a number of factors, including water content, fat content, protein content, and other the presence of other ingredients such as salt, acid, and stabilizers. Meltability may be measured with a rapid visco analyzer (RVA) (Metzger et al., 2002, RVA: Process cheese manufacture, Aust. J Dairy Technol. 57:136; Kapoor et al., 2004, Comparison of pilot scale and rapid visco analyzer process cheese manufacture, J. Dairy Sci. 87:2813-2821; Prow et al., 2005, Melt analysis of process cheese spread or product using a rapid visco analyzer, J. Dairy Sci. 88:1277-1287). Meltability may also be measured by the Schreiber melt test (1977), wherein a 0.5 cm high plug of cheese is placed in a glass petri dish and heated in an oven at 450° F. for 5 minutes. Other melting tests include the Arnott test (1957), the tube test (1958), the melt analysis/UW meltmeter (1997), and the Dynamic Stress Rheometry (DSR) (1998). Shown in Table 17 are some examples of cheeses and their melting temperatures. In some embodiments, the cheese compositions described herein have a melting temperature similar to one or more of the cheeses in Table 17. In some embodiments, the cheese compositions described herein have a melting temperature in the range of 100° F. to 200° F., such as about 120° F., 130° F., 150° F., or 180° F.

TABLE 17
Melting ranges for cheese

| | Melt | |
|---|---|---|
| Cheese type | temperature | Examples |
| Process cheese | 120° F./49° C. | Pasteurized Process Cheese |
| Soft or semi-soft | 130° F./54° C. | Mozzarella |
| cheese | | |
| Hard cheese | 150° F./66° C. | Cheddar, Colby, Edam, Swiss, Gouda |
| Very hard cheese | 180° F./82° C. | Parmesan, Romano, Sardo, Grana |

In some embodiments, the cheese composition has a melting point of about 35° C. to about 100° C. In some embodiments, the cheese composition has a melting point of about 40° C. to about 50° C. In some embodiments, the cheese composition has a melting point of about 50° C. to about 60° C. In some embodiments, the cheese composition has a melting point of about 60° C. to about 70° C. In some embodiments, the cheese composition has a melting point of about 70° C. to about 90° C.

As mentioned above, the properties of cheese can be influenced by a number of factors, such as lipids, salts, and/or calcium. Lipids that may be added to the cheese compositions disclosed herein include, for example, dairy fats or vegetable oils such as palm oil or palm kernel oil, butter oil, anhydrous milkfat, soybean oil, corn oil, rapeseed oil, canola oil, sunflower oil, safflower oil, coconut oil, rice bran oil, olive oil, sesame oil, flaxseed oil, hemp oil, cottonseed oil, peanut oil, almond oil, beech nut oil, Brazil nut oil, cashew oil, hazelnut oil, macadamia oil, mongongo nut oil, pecan oil, pine nut oil, pistachio oil, walnut oil, pumpkin seed oil, grapefruit seed oil, lemon oil, apricot oil, apple seed oil, argan oil, avocado oil, or orange oil.

Examples of salts that may be included in a cheese composition include, but are not limited to, magnesium chloride, sodium chloride, calcium chloride, sodium phosphates and trisodium citrate. In some embodiments, a cheese composition comprises at least one lipid and at least one salt. In some embodiments, a cheese composition comprises calcium. In some embodiments, a cheese composition comprises calcium at a concentration of about 0 to about 2% by weight. In some embodiments, a cheese composition comprises calcium at a concentration of about 0.001 to about 2% by weight. In some embodiments, a cheese composition comprises calcium at a concentration of about 0.01 to about 2% by weight. In some embodiments, a cheese composition comprises calcium at a concentration of about 0.1 to about 2% by weight. In some embodiments, a cheese composition comprises calcium at a concentration of about 1 to about 2% by weight. In some embodiments, a cheese composition has a pH of about 5.2 to about 5.9. In some embodiments, a cheese composition comprises at least one organoleptic property similar to cheese (i.e., cheese produced using mammalian milk, such as bovine milk or goat milk) selected from the group consisting of taste, appearance, mouthfeel, structure, texture, density, elasticity, springiness, coagulation, binding, leavening, aeration, foaming, creaminess, and emulsification. In some embodiments, the cheese composition comprises at least two organoleptic properties similar to cheese (i.e., cheese produced using mammalian milk, such as bovine milk or goat milk) selected from the group consisting of taste, appearance, mouthfeel, structure, texture, density, elasticity, springiness, coagulation, binding, leavening, aeration, foaming, creaminess, and emulsification. In some embodiments, the cheese composition comprises at least three organoleptic properties similar to cheese (i.e., cheese produced using mammalian milk, such as bovine milk or goat milk) selected from the group consisting of taste, appearance, mouthfeel, structure, texture, density, elasticity, springiness, coagulation, binding, leavening, aeration, foaming, creaminess, and emulsification.

In some embodiments, a cheese composition comprises one or more vitamins, such as retinal, carotene, vitamins, vitamin D, vitamin E, vitamin B12, thiamin, or riboflavin.

### Colloidal Suspensions Comprising One or More Isolated or Recombinant Casein Proteins

A colloidal suspension is a mixture having particles suspended in a continuous phase with another component. The particles may be, for example, proteins. The other component may be, for example water. Many different kinds of foods may be colloidal suspensions, including beverages and other foods such as jam, ice cream, tofu, mayonnaise, etc. One example of a colloidal suspension is milk.

The colloidal suspensions described herein may be a Newtonian fluid or a non-Newtonian fluid. Newtonian fluids are characterized by a viscosity that is independent of shear rate; they follow Newton's law of viscosity. Apparent viscosity is the shear stress applied to a fluid divided by the shear rate (expressed in Pascal-second or centipoise units). For a Newtonian fluid, the apparent viscosity is constant. Water is an example of a Newtonian fluid. Non-Newtonian fluids do not follow Newton's law of viscosity; their viscosity can change (for example, become more liquid or more solid) when under force. Ketchup is an example of a non-Newtonian fluid.

In some embodiments, a colloidal suspension comprises: 1-4 milk proteins (i.e., 1, 2, 3, or 4 recombinant milk proteins). The milk proteins may be recombinant or may be isolated from a mammalian milk. In some embodiments, the milk proteins may be plant-expressed.

In some embodiments, a colloidal suspension comprises recombinant beta-casein and at least one lipid and does not contain an organoleptically functional amount of beta-lactoglobulin. In some embodiments, the colloidal suspension does not comprise any additional casein proteins. In some embodiments, the colloidal suspension comprises at least one additional casein protein. In some embodiments, the at least one additional casein protein is selected from kappa-casein, para-kappa-casein, beta-casein, alpha-S1-casein and alpha-S2-casein. In some embodiments, the at least one additional casein protein is kappa-casein or para-kappa-casein. In some embodiments, the colloidal suspension is a non-Newtonian fluid.

In some embodiments, at least 80%, at least 90%, or at least 95% by weight of the total casein protein in a colloidal suspension is beta-casein. In some embodiments, the beta-casein is expressed in a plant. In some embodiments, the beta-casein is expressed in a soybean plant. In some embodiments, all caseins in the composition are plant expressed. In some embodiments, the composition comprises a fusion protein comprising recombinant beta-casein.

In some embodiments, a colloidal suspension is a non-Newtonian fluid. In some embodiments, a colloidal suspension is characterized as a shear thinning fluid with an apparent viscosity greater than 10 centipoise, at a shear rate of 1 sec⁻¹. In some embodiments, the suspension is an aqueous suspension.

In some embodiments, the milk proteins comprise between 0.5% (w/v) to 15% (w/v) of the composition, such as about 0.5% (w/v); about 1.0% (w/v), about 1.5% (w/v), about 2.0% (w/v), about 2.5% (w/v), about 3.0% (w/v), about 3.5% (w/v), about 4.0% (w/v), about 4.5% (w/v), about 5.0% (w/v), about 5.5% (w/v), about 6.0% (w/v), about 6.5% (w/v), about 7.0% (w/v), about 7.5% (w/v), about 8.0% (w/v), about 8.5% (w/v), about 9.0% (w/v), about 9.5% (w/v), about 10.1% (w/v), about 10.5% (w/v), about 11.0% (w/v), about 11.5% (w/v), about 12.0% (w/v), about 12.5% (w/v), about 13.0% (w/v), about 13.5% (w/v), about 13.0% (w/v), about 14.5% (w/v), or about 15.0% (w/v). In some embodiments, the colloidal suspension may comprise one or more additional components, such as ash. In some embodiments, the colloidal suspension may comprise one or more vitamins such as retinal, carotene, vitamins, vitamin D, vitamin E, vitamin B12, thiamin, or riboflavin.

In some embodiments, a colloidal suspension comprises about 8% (w/v) to about 25% (w/v) total protein, such as about 8% to about 10%, about 10% to about 15%, about 15% to about 20%, or about 20 to about 25% total protein. In some embodiments, a colloidal suspension comprises about 1% to about 10% (w/v) total protein. In some embodiments, a colloidal suspension comprises about 25% to about 35%, about 35% to about 45%, about 45% to about 55%, about 55% to about 65%, about 65% to about 75% (w/v), or more total protein.

In some embodiments, about 1% to about 5% of the total protein in the colloidal suspension is casein protein. In some embodiments, about 5% to about 10% of the total protein in the colloidal suspension is casein protein. In some embodiments, about 10% to about 20% of the total protein in the colloidal suspension is casein protein. In some embodiments, about 20% to about 30% of the total protein in the colloidal suspension is casein protein. In some embodiments, about 30% to about 40% of the total protein in the colloidal suspension is casein protein. In some embodiments, about 40% to about 50% of the total protein in the colloidal suspension is casein protein. In some embodiments, about 50% to about 60% of the total protein in the colloidal suspension is casein protein. In some embodiments, about 60% to about 70% of the total protein in the colloidal suspension is casein protein. In some embodiments, about 70% to about 80% of the total protein in the colloidal suspension is casein protein. In some embodiments, about 80% to about 90% of the total protein in the colloidal suspension is casein protein. In some embodiments, about 90% to about 100% of the total protein in the colloidal suspension is casein protein.

In some embodiments, at least 1%, at least 2%, at least 3%, at least 4%, at least 5%, at least 6%, at least 7%, at least 8%, at least 9%, at least 10%, at least 110, at least 12%, at least 13%, at least 14%, at least 15%, at least 16%, at least 17%, at least 18%, at least 19%, at least 20%, or more of the total protein in the colloidal suspension is casein protein.

In some embodiments, about 20% to about 100% of the casein protein in the colloidal suspension is kappa casein. For example, the colloidal suspension may comprise about 20% to about 30%, about 30% to about 40%, about 40% to about 50%, about 50% to about 60%, about 60% to about 70%, about 70% to about 80%, about 80% to about 90%, or about 90% to about 100% kappa casein. In some embodiments, about 20% to about 30%, about 30% to about 40%, about 40% to about 50%, about 50% to about 60%, about 60% to about 70%, about 70% to about 80%, about 80% to about 90%, or about 90% to about 100% of the casein protein in the colloidal suspension is kappa casein.

In some embodiments, about 20% to about 100% of the casein protein in the colloidal suspension is para-kappa casein. For example, the colloidal suspension may comprise about 20% to about 30%, about 30% to about 40%, about 40% to about 50%, about 50% to about 60%, about 60% to about 70%, about 70% to about 80%, about 80% to about 90%, or about 90% to about 100% para-kappa casein. In some embodiments, about 20% to about 30%, about 30% to about 40%, about 40% to about 50%, about 50% to about 60%, about 60% to about 70%, about 70% to about 80%, about 80% to about 90%, or about 90% to about 100% of the casein protein in the colloidal suspension is para-kappa casein.

In some embodiments, about 20% to about 100% of the casein protein in the colloidal suspension is beta casein. In some embodiments, about 50% to about 100% of the casein protein in the colloidal suspension is beta casein. For example, the colloidal suspension may comprise about 20% to about 30%, about 30% to about 40%, about 40% to about 50%, about 50% to about 60%, about 60% to about 70%, about 70% to about 80%, about 80% to about 90%, or about 90% to about 100% beta casein. In some embodiments, about 20% to about 30%, about 30% to about 40%, about 40% to about 50%, about 50% to about 60%, about 60% to about 70%, about 70% to about 80%, about 80% to about 90%, or about 90% to about 100% of the casein protein in the colloidal suspension is beta casein.

In some embodiments, about 20% to about 100% of the casein protein in the colloidal suspension is alpha-S1-casein. In some embodiments, about 50% to about 100% of the casein protein in the colloidal suspension is alpha-S1-casein. For example, the colloidal suspension may comprise about 20% to about 30%, about 30% to about 40%, about 40% to about 50%, about 50% to about 60%, about 60% to about 70%, about 70% to about 80%, about 80% to about 90%, or about 90% to about 100% alpha-S1-casein. In some embodiments, about 20% to about 30%, about 30% to about 40%, about 40% to about 50%, about 50% to about 60%, about 60% to about 70%, about 70% to about 80%, about 80% to about 90%, or about 90% to about 100% of the casein protein in the colloidal suspension is alpha-S1-casein.

In some embodiments, about 20% to about 100% of the casein protein in the colloidal suspension is alpha-S2-casein. In some embodiments, about 50% to about 100% of the casein protein in the colloidal suspension is alpha-S2-casein. For example, the colloidal suspension may comprise about 20% to about 30%, about 30% to about 40%, about 40% to about 50%, about 50% to about 60%, about 60% to about 70%, about 70% to about 80%, about 80% to about 90%, or about 90% to about 100% alpha-S2-casein. In some embodiments, about 20% to about 30%, about 30% to about 40%, about 40% to about 50%, about 50% to about 60%, about 60% to about 70%, about 70% to about 80%, about 80% to about 90%, or about 90% to about 100% of the casein protein in the colloidal suspension is alpha-S2-casein.

In some embodiments, colloidal suspension has at least one organoleptic property that is substantially similar to bovine milk. In some embodiments, the organoleptic property is selected from the group consisting of taste, appearance, mouthfeel, structure, texture, density, elasticity, springiness, coagulation, binding, leavening, aeration, foaming, creaminess, and emulsification. In some embodiments, colloidal suspension has at least two, at least three, at least four, at least five, or more organoleptic properties that are substantially similar to bovine milk. In some embodiments, the plant-expressed milk proteins are recombinant, and are selected from beta lactoglobulin, kappa-casein, para-kappa-casein, beta-casein, alpha-S1-casein, and alpha-S2-casein.

In some embodiments, the colloidal suspensions described herein may be used to produce one or more food compositions such as butter, ice cream, frozen yogurt or custard, yogurt, frozen desserts, cottage cheese, cream cheese, curds, and crème fraiche.

### Methods for Making the Food Compositions Described Herein

Also provided herein are methods for making solid phase, protein-stabilized emulsions, colloidal suspensions, dairy alternatives and food compositions described herein (collectively referred to in this section as “compositions”). In some embodiments, a method for making a composition comprises isolating one or more casein proteins from a mammalian milk. In some embodiments, a method for making a composition comprises expressing a casein protein in a cell (e.g., in a plant, or microorganism), extracting the recombinant protein, and preparing a composition comprising recombinant casein protein (See, e.g., FIG. 13).

Initially, all ingredients for the composition are provided. For example, in some embodiments, the one or more milk proteins are provided. The milk proteins may be isolated from a mammalian milk, or may be produced recombinantly (e.g., by expression in a plant). An illustrative process for preparing a recombinant protein for use in making a composition as described herein is illustrated in FIG. 13 and is also described below. In some embodiments, one or more lipids, salts, acids, etc. are also provided. In some embodiments, ash is provided. In some embodiments, one or more vitamins is provided, such as retinal, carotene, vitamins, vitamin D, vitamin E, vitamin B12, thiamin, or riboflavin.

The ingredients are then combined and mixed. In some embodiments, the mixing is performed at a pre-determined temperature, for example a temperature in the range of about 0° C. to about 10° C., about 10° C. to about 20° C., about 20° C. to about 40° C., about 40° C. to about 50° C., about 50° C. to about 60° C., about 60° C. to about 70° C., about 70° C. to about 80° C., about 80° C. to about 90° C., about 90° C. to about 100° C. or higher. In some embodiments, the mixing is performed at a temperature of about 40° C. In some embodiments, the mixing is performed at a temperature of about 85° C. In some embodiments, the mixing is performed at a temperature of about 90° C. In some embodiments, the mixing is performed at a temperature of about 95° C. In some embodiments, the mixing is performed at a speed that will not negatively affect the properties of the composition, such as a speed of about 100 RPM, 200 RPM, 300 RPM, 400 RPM, 500 RPM, 600 RPM, 700 RPM, 800 RPM, 900 RPM, 1000 RPM, or more. In some embodiments the mixing lasts for about 1 minute, about 2 minutes, about 3 minutes, about 4 minutes, about 5 minutes, about 6 minutes, about 7 minutes, about 8 minutes, about 9 minutes, about 10 minutes, or more.

In some embodiments, the composition is mixed only once. In some embodiments, the composition is mixed more than once, such as twice, three times, four times, five times, or more. In some embodiments, the temperature is changed between each mix. For example, in some embodiments, the composition is mixed a first time at a first temperature, and a second time at a second temperature. In some embodiments, the composition is mixed a first time at a first temperature, a second time at a second temperature, and a third time at a third temperature. In some embodiments, the composition is mixed a first time at a first temperature, a second time at a second temperature, a third time at a third temperature, and a fourth time at a fourth temperature. In some embodiments, the composition is mixed a first time at 40° C., a second time at 95° C., a third time at 90° C., and a fourth time at 85° C. After mixing and/or between different mixings the composition my be allowed to rest.

The compositions are then poured into molds. The molds may be of any shape, such as cube-shaped, cylindrical-shaped, triangular prism-shaped, spherical-shaped, cone-shaped, or rectangular prism-shaped. The compositions may then be covered, cooled and stored. In some embodiments, the compositions may be stored for at least 1 day, at least 3 days, at least 5 days, at least 7 days, at least 30 days, at least 180 days, or at least 360 days.

The pH of the composition may be monitored during production thereof. In some embodiments, the pH may be adjusted to a target pH, such as a pH in the range of about 5.5 to about 5.7. As will be understood by those of ordinary skill in the art, the pH may be adjusted up or down using acids or bases. Exemplary acids that may be used to adjust the pH include lactic acid, citric acid, or sodium citrate.

An illustrative method for preparing a food composition of the disclosure is provided in FIG. 13. The first step in this method is production of a seed expressing a fusion protein. In this process, an expression construct is designed. The construct is then transformed into a plant. The plant is grown under conditions that allow for expression of the fusion protein. Subsequently, seeds may be collected from the plant for further processing.

The next step in the method for preparing a food composition illustrated in FIG. 13 is seed processing, to prepare one or more ingredients for use in a food composition. First, the seeds are hulled and ground. Protein (including the fusion protein and other seed proteins) is extracted from the seed. The protein fraction may then be enriched. Specifically, the protein fraction may be enriched for fusion protein. Optionally, the fusion protein may then be concentrated.

The plant protein, including fusion proteins, may be extracted from a plant using standard methods known in the art. For example, the proteins may be extracted using solvent or aqueous extraction. In some embodiments, the oil may be separated from the proteins using hexane or ethanol extraction to produce a white flake. The proteins may be extracted from the white flake using controlled temperature in an aqueous buffered environment (e.g., carbonate, citrate), in order to control the pH. The fusion proteins can be separated from the plant proteins using selective precipitation of one or more of the proteins with centrifugation or filtration methods. In some embodiments, one or more additives may be used to aid the extraction processes (e.g., salts, protease/peptidase inhibitors, osmolytes, solvents, reducing agents, etc.) The following step is processing the fusion protein into a food product. In some embodiments, constituent proteins of the fusion protein may be separated from one another before they are used to formulate a product. In some embodiments, only one of the constituent proteins of the fusion protein is used in the product. In some embodiments, more than one of the constituent proteins of the fusion protein is used in the product. In some embodiments, all of the constituent proteins of the fusion protein may be used in the product. In some embodiments, the fusion protein may be used itself in the food product. The product is then formulated as desired.

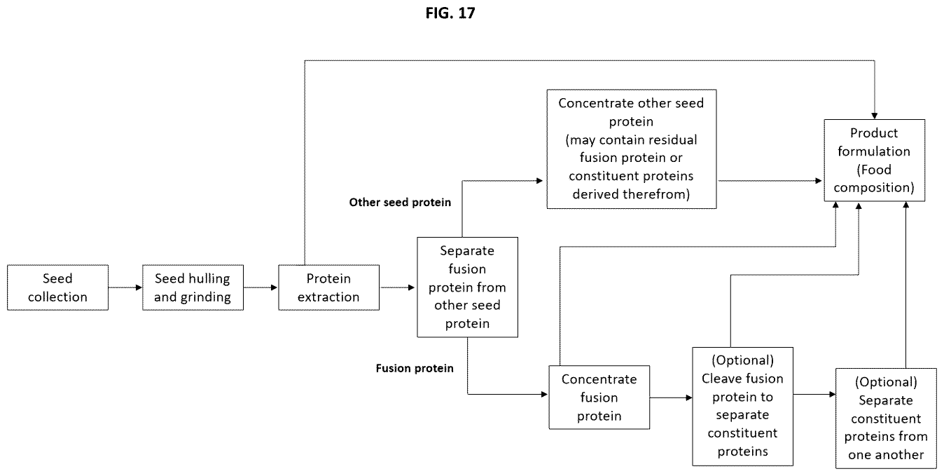

FIG. 17 also illustrates a method for preparing a food composition. In this method, after seeds are collected, hulled and ground, and protein has been extracted, the fusion protein is separated from other seed protein. In some embodiments, this separation is not 100% efficient, meaning that the “other seed protein” fraction may still contain some residual fusion protein. For example, in some embodiments, the other seed protein fraction may comprise about 0.1%, about 0.3%, about 0.5%, about 0.7%, about 1%, about 2%, about 3%, about 4%, about 5%, about 6%, about 7%, about 8%, about 9%, about 10%, about 20%, about 30%, or about 50% fusion protein by weight. The other seed protein fraction may then be used directly in a food composition. Alternatively, the other seed protein fraction may be combined with concentrated fusion protein. In some embodiments, the other seed protein fraction is combined with one or more of the constituent proteins from the fusion protein. In some embodiments, the other seed protein fraction is combined with all of the constituent proteins from the fusion protein.

It may be advantageous to use a seed processing composition comprising plant protein and a fusion protein (e.g., about 0.1%, about 0.3%, about 0.5%, about 0.7%, about 1%, about 2%, about 3%, about 4%, about 5%, about 6%, about 7%, about 8%, about 9%, about 10%, about 20%, about 30%, or about 50% fusion protein by weight) as an ingredient in a food composition. Using both (i) a fusion protein produced by a seed and (ii) other protein extracted from the seed allows for efficient use of resources and reduces waste. Such processes may simplify food manufacturing processes, and reduce the unit cost to manufacture each product. Thus, provided herein is a method of making a food composition, the method comprising: (i) expressing a fusion protein in a transformed plant; and (ii) preparing a food composition comprising the fusion protein and plant protein from the same transformed plant in which the fusion protein was produced. In some embodiments, the transformed plant is a soybean. In some embodiments, the transformed plant is pea.

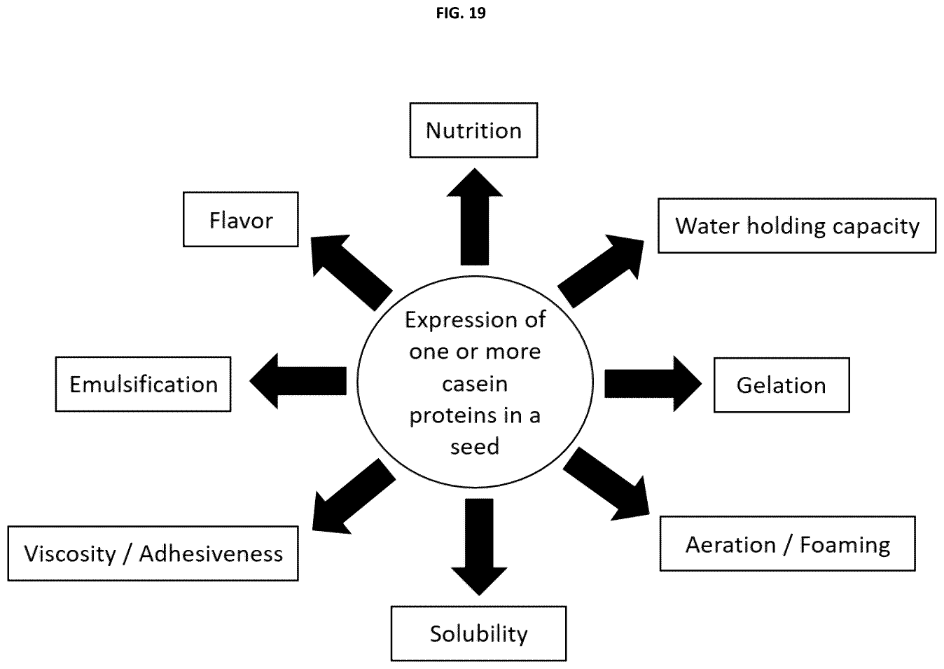

Without being bound by any theory, it is believed that having a casein protein (i.e., as a monomer or as part of a fusion protein) in a plant protein composition may improve the properties of the plant protein composition. FIG. 19 illustrates various properties that may be improved due to the presence of one or more caseins in a plant protein composition, including. In some embodiments, a plant protein composition comprising one or more casein proteins has improved nutritional properties compared to a plant protein composition that does not contain a casein protein. In some embodiments, a plant protein composition comprising one or more casein proteins has improved organoleptic properties, such as taste, compared to a plant protein composition that does not contain a casein protein. In some embodiments, a plant protein composition comprising one or more casein proteins has improved water holding capacity compared to a plant protein composition that does not contain a casein protein. In some embodiments, a plant protein composition comprising one or more casein proteins has improved emulsification compared to a plant protein composition that does not contain a casein protein. In some embodiments, a plant protein composition comprising one or more casein proteins has improved gelation compared to a plant protein composition that does not contain a casein protein. In some embodiments, a plant protein composition comprising one or more casein proteins has improved viscosity and/or adhesiveness compared to a plant protein composition that does not contain a casein protein. In some embodiments, a plant protein composition comprising one or more casein proteins has improved aeration and/or foaming compared to a plant protein composition that does not contain a casein protein. In some embodiments, a plant protein composition comprising one or more casein proteins has improved solubility compared to a plant protein composition that does not contain a casein protein. Illustrative improvements in each one of these properties are described in further detail below.

Nutrition: The presence of a casein protein (alone or expressed as a fusion protein) in the plant protein composition may enhance the nutritional properties of the plant protein composition and/or any food compositions comprising the plant protein composition. For example, the presence of the casein protein may, in some embodiments, improve the balance of essential amino acids. Pea protein has a PDCAAS (protein digestibility corrected amino acid score) of about 0.82. Nutritionally complete proteins have a score of about 1.0. By expressing a casein protein (fused to, for example, ovalbumin and/or beta-lactoglobulin) at sufficient levels in a pea plant, the PDCAAS of the protein extracted from the pea plant may reach 1.0, provided that the limiting amino acids (e.g., methionine) are raised. In some embodiments, a plant protein composition comprising a casein protein comprises a PDCAAS of about 0.90, about 0.95, about 1.0, or about 1.05.

Gelation: In some embodiments, the casein protein present in the plant protein composition may enhance gelation of the plant protein composition and/or any food compositions comprising the plant protein composition. Many of the proteins used as fusion partners in the fusion proteins described herein, including whey proteins (e.g., beta-lactoglobulin) and egg proteins are often added to a number of food products such as meats and bakery products, because the proteins gel after heating and cooling. Seed proteins are generally insoluble under the processing conditions used to prepare many foods, such as meats and bakery products. Methylcellulose is often added to plant-based meats to impart gelling, and egg white has historically been used in some vegetarian products. However, eggs are not considered vegan and do not meet the standard of “plant-based” for many individuals. Thus, by using a plant composition comprising one or more casein proteins (fused to, for example, an egg protein and/or a whey protein), enhanced gelation may be achieved without using animal products.

Solubility: In some embodiments, the casein protein present in the plant protein composition may enhance solubility of the plant protein composition and/or any food compositions comprising the plant protein composition. Seed proteins typically have poor solubility at acidic and neutral pH. Beverage formulations are suspensions utilizing hydrocolloids such as gellan gum to keep the proteins from settling out. Conversely, casein proteins are soluble at neutral pH, and whey proteins are soluble at acidic pH. Both caseins and whey are soluble at neutral pH. In some embodiments, beverages made with seed protein enhanced by the expression of casein proteins (expressed alone or fused to, for example, a whey protein) exhibit a smoother and/or less chalky mouthfeel.

Emulsification: In some embodiments, the casein protein present in the plant protein composition may enhance emulsification of the plant protein composition and/or any food compositions comprising the plant protein composition. Caseinates are effective at emulsifying lipids with a low viscosity, and this property is used in spray drying to produce powdered coffee creamers and powdered sauces with lipids used in convenience foods. Seed proteins do not have these attributes, and additives such as starches chemically modified with octenyl succinic anhydride are often used as additives in plant protein compositions. Food compositions made with plant protein compositions comprising casein proteins will have improved emulsification properties for a number of different applications.

Water holding capacity: In some embodiments, the casein protein may enhance the water holding capacity of the plant protein. During the processing of the plant protein, pH and heat conditions can be modified to denature the casein protein to enhance this property.

Aeration/Foaming: Aeration and foaming properties of the plant protein can be improved by the addition of the casein proteins. Caseins have excellent foaming properties, as evidenced by their incorporation in frozen whipped toppings. Egg proteins and beta-lactoglobulin also demonstrate good foaming properties. The surface-active properties of these proteins are beneficial in food compositions.

Viscosity/Adhesiveness: Unstructured casein proteins can unfold to interact with other components of a food composition to impart viscosity and adhesiveness. Granola bars can utilize casein proteins at specific concentrations to form a viscous solution that holds the particulates together.

Flavor: The casein proteins can also improve the flavor of plant proteins. In addition to acting as binders for off flavors, casein proteins can impart desirable flavors to food compositions. Hydrolyzed protein caseins impart a savory umami flavor similar to those from autolyzed yeast extract. Some of the expressed caseins are hydrolyzed by plant enzymes in the seed, and the resultant peptides can provide savory flavors.

In some embodiments, a plant protein composition comprising a fusion protein is used to produce a food composition. The food composition may be, for example, a meat analog, a nutritional bar, a bakery product, a beverage, mashed potatoes, or candy. In some embodiments, the food composition is for a human. For example, the food composition may be infant formula. In some embodiments, the food composition is for a companion animal (e.g., a dog, cat, rabbit, hamster, guinea pig, horse, etc.) For example, the food composition may be pet food.

Also provided herein are various compositions prepared during a method of making a food composition. For example, in some embodiments, a seed processing composition is provided. In some embodiments, a seed processing composition comprises (a) a fusion protein comprising i) a full-length κ-casein or para-κ-casein component; and ii) a β-lactoglobulin component; and (b) plant seed tissue. In some embodiments, a seed processing composition comprises (a) a fusion protein comprising i) a beta-casein component; and ii) a β-lactoglobulin component; and (b) plant seed tissue. In some embodiments, a seed processing composition comprises (a) a fusion protein comprising i) a milk protein (e.g., a casein protein); and ii) a second protein (i.e., a fusion partner); and (b) plant seed tissue. In some embodiments, the plant seed tissue is ground. In some embodiments, the plant seed tissue is from soybean. In some embodiments, the seed processing composition comprises at least one member selected from the group consisting of: enzyme (e.g., chymosin), protease, extractant, solvent (e.g., ethanol, or hexane), buffer, additive, salt, protease inhibitor, peptidase inhibitor, osmolyte, and reducing agent.

In some embodiments, a protein concentrate composition is provided. In some embodiments, the protein concentrate composition comprises: a fusion protein, comprising i) a full-length κ-casein or para-κ-casein component; and ii) a β-lactoglobulin component. In some embodiments, the protein concentrate composition comprises: a fusion protein, comprising i) a beta-casein component; and ii) a β-lactoglobulin component. In some embodiments, the protein concentrate composition comprises: a fusion protein, comprising i) a milk protein (e.g., a casein protein); and ii) a second protein (i.e., a fusion partner). In some embodiments, the fusion protein is present in an enriched amount, relative to other components present in the composition. In some embodiments, there is substantially no plant seed tissue present in the protein concentrate composition. In some embodiments, the protein concentrate composition further comprises at least one member selected from the group consisting of: enzyme (e.g., chymosin), protease, extractant, solvent (e.g., ethanol, or hexane), buffer, additive, salt, protease inhibitor, peptidase inhibitor, osmolyte, and reducing agent.

In some embodiments, a food composition comprises a fusion protein comprising a first protein and a second protein. In some embodiments, a food composition comprises a first protein, wherein the first protein is derived from (i.e., separated from) a fusion protein comprising at least the first protein and a second protein. In some embodiments, a food composition comprises (i) a fusion protein comprising a first protein and a second protein and (ii) at least one of the first protein and the second protein, wherein the first protein and/or the second protein has been separated from the fusion protein. The first protein and/or second protein which have been separated from the fusion protein may comprise, in some embodiments, at least at least one non-native amino acid from an introduced protease cleavage site (e.g., a chymosin cleavage site).

In some embodiments, the food composition is a solid. In some embodiments, the food composition is a liquid. In some embodiments, the food composition is a powder.

In some embodiments, the food composition is a solid phase, protein-stabilized emulsion. In some embodiments, the food composition is a colloidal suspension.

In some embodiments, the fusion proteins and transgenic plants described herein may be used to prepare a food composition such as cheese or processed cheese products. In some embodiments, the food composition is an alternative dairy composition selected such as milk, cream, or butter. The alternative milk composition may be used to prepare alternative dairy compositions such as yogurt and fermented dairy products, directly acidified counterparts of fermented dairy products, cottage cheese, dressing, curds, crème fraiche, toppings, tofu, icings, fillings, low-fat spreads, dairy-based dry mixes, frozen dairy products, frozen desserts, desserts, baked goods, soups, sauces, salad dressing, geriatric nutrition, creams and creamers, analog dairy products, follow-up formula, baby formula, infant formula, milk, dairy beverages, acid dairy drinks, smoothies, milk tea, butter, margarine, butter alternatives, growing up milks, low-lactose products and beverages, medical and clinical nutrition products, protein/nutrition bar applications, sports beverages, confections, meat products, analog meat products, meal replacement beverages, and weight management food and beverages.

In some embodiments the fusion proteins and transgenic plants described herein may be used to prepare a dairy product. In some embodiments, the dairy product is a fermented dairy product. An illustrative list of fermented dairy products includes cultured buttermilk, sour cream, yogurt, skyr, leben, lassi, or kefir. In some embodiments the fusion proteins and transgenic plants described herein may be used to prepare cheese products.

In some embodiments the fusion proteins and transgenic plants described herein may be used to prepare a powder containing a milk protein. In some embodiments, the fusion proteins and transgenic plants described herein may be used to prepare a low-lactose product.

In some embodiments, a method for making a food composition comprises, expressing a recombinant fusion protein of the disclosure in a plant, extracting the recombinant fusion protein from the plant, optionally separating the milk protein from the mammalian or plant protein, and creating a food composition using the fusion protein and/or the milk protein.

In some embodiments, a method of expressing, extracting, and making a food composition from a fusion protein, comprises: expressing a fusion protein in a host cell, the fusion protein comprising a first protein and a second protein; extracting the fusion protein from the host cell; and processing the fusion protein into a food composition. The food composition may be, for example, cheese, processed cheese product, yogurt, fermented dairy product, directly acidified counterpart of fermented dairy product, cottage cheese dressing, frozen dairy product, frozen dessert, dessert, baked good, topping, icing, filling, low-fat spread, dairy-based dry mix, soup, sauce, salad dressing, geriatric nutrition, cream, creamer, analog dairy product, follow-up formula, baby formula, infant formula, milk, tofu, dairy beverage, acid dairy drink, smoothie, milk tea, butter, margarine, butter alternative, growing up milk, low-lactose product, low-lactose beverage, medical and clinical nutrition product, protein bar, nutrition bar, sport beverage, confection, meat product, analog meat product, meal replacement beverage, weight management food and beverage, dairy product, cultured buttermilk, sour cream, yogurt, skyr, leben, lassi, kefir, powder containing a milk protein, and low-lactose product. In some embodiments, the food composition is a dairy product. In some embodiments, the food composition is a cheese.

In some embodiments, a method for making a food composition comprises, expressing a recombinant fusion protein of the disclosure in a plant, extracting one or both of the proteins, and creating a food composition using the milk protein. In some embodiments, the first protein and the second protein are separated from one another in the plant cell, prior to extraction. In some embodiments, the first protein is separated from the second protein after extraction, for example by contacting the fusion protein with an enzyme that cleaves the fusion protein. The enzyme may be, for example, chymosin. In some embodiments, the fusion protein is cleaved using rennet.

Composition of food compositions described herein

Provided is a composition that comprises at least a recombinant mammalian milk protein and a second protein selected from the group consisting of 7S globulin glycinin, 11S globulin, Lipoxygenase, and a Kunitz Trypsin Inhibitor. In some embodiments, the second protein is the 7S globulin glycinin. In some embodiments, the second protein is the 11S globulin. In some embodiments, the second protein is the Kunitz Trypsin Inhibitor. In some embodiments, the composition further comprises chlorophyll.

Any of the described milk proteins can be utilized in the aforementioned composition. In some embodiments, the milk protein is selected from the group consisting of α-S1 casein, α-S2 casein, β-casein, κ-casein, para-κ-casein, and β-lactoglobulin. In some embodiments, the milk protein is selected from the group consisting of: α-S1 casein, α-S2 casein, β-casein, κ-casein, para-κ-casein, β-lactoglobulin, α-lactalbumin, lysozyme, lactoferrin, lactoperoxidase, serum albumin, and an immunoglobulin. In some embodiments, the composition has reduced or no lactose. In some embodiments, the composition lacks lactose. In some embodiments, the composition does not have one or more proteins present in cow milk or another mammalian milk (e.g., goat, sheep, human, and combinations thereof). In some embodiments, the one or more proteins present in cow milk are selected from the group consisting of α-S1 casein, α-S2 casein, β-casein, κ-casein, para-κ-casein, β-lactoglobulin, α-lactalbumin, lysozyme, lactoferrin, lactoperoxidase, serum albumin, and an immunoglobulin. In some embodiments, the one or more proteins present in cow milk are selected from the group consisting of α-lactalbumin, lysozyme, lactoferrin, lactoperoxidase, serum albumin, and an immunoglobulin.

In some embodiments, a recombinant milk protein is expressed in a fusion protein with a fusion partner. Potential fusion partners are described herein. In some embodiments, the fusion partner comprises a milk protein. In some embodiments, a fusion partner is selected from the group consisting of α-S1 casein, α-S2 casein, β-casein, κ-casein, para-κ-casein, and β-lactoglobulin. In some embodiments, a fusion partner is selected from the group consisting of: α-S1 casein, α-S2 casein, β-casein, κ-casein, para-κ-casein, β-lactoglobulin, α-lactalbumin, lysozyme, lactoferrin, lactoperoxidase, serum albumin, and an immunoglobulin

In some embodiments, a fusion protein comprises a configuration comprising: Beta-casein-AlphaS1-casein-AlphaS1-casein-Beta-casein; Beta-casein-Beta-casein-Kappa-casein-Beta-lactoglobulin; Beta-casein-Beta-casein-Beta-casein-Beta-casein; and Gamma-Zein-Beta-casein.

In some embodiments, a composition comprises a nucleic acid encoding glycinin. In some embodiments, a composition comprises a nucleic acid encoding lipoxygenase. In some embodiments, a composition comprises a nucleic acid encoding kunitz trypsin inhibitor. In some embodiments, a recombinant mammalian milk protein comprises reduced phosphorylation as compared to a corresponding mammalian milk protein in a bovine as determined by mass spectrometry after trypsin or chymotrypsin digestion.

In some embodiments, the milk protein and second protein are present at a w/w ratio of at least about 1:100, 1:90, 1:80, 1:70, 1:60, 1:50, 1:40, 1:30, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, or 1:1. In some embodiments, the milk protein and second protein are present at a w/w ratio of at least about: 1: 50-1:100, 1:20-1:50, 1:1-1:10, 1:1-1:20, 1:10-1:30, 1:20-1:50, 1:15-1:30, 1:20-1:30, 1:30-1:40, or 1:16-1:20.

In some embodiments, traditional plant products with no milk proteins, or with low ratios of milk protein to secondary protein, were not suitable for their intended purpose. For example, lack of milk proteins resulted in food compositions that lacked stability in aqueous solutions, separated out, and also lacked nutritional qualities associated with milk products.

In contrast, the compositions of the present disclosure, which selected rations of first protein to second protein exhibit improved qualities. Example 28 tests a few of these improved compositions and demonstrates their improved physical and organoleptic properties.

In some embodiments, the composition is a plant-based milk, ice cream, tofu, cheese, spread, and the like. In some embodiments, the composition requires little to no processing. For example, in some embodiments the compositions exhibiting the indicated ratios are edible seeds, legumes, or other plant parts (e.g., crushed up seed paste). In some embodiments, the compositions exhibiting the indicated ratios are soluble portions of disrupted tissue, such as soy milk.

### Plant Seed Proteins

Many plant storage tissues, such as seeds, leaves, roots, and tubers, accumulate reserves of proteins during development. Certain types of seeds, such as soybean, are capable of accumulating a relatively high level of endogenous protein, about 35% to 55% protein of the dry weight, depending on varieties. The abundance of proteins in soybean seeds has made them the primary dietary protein source and has stimulated an interest in developing approaches to genetically engineer seeds to improve their nutritional quality or produce heterologous proteins of interest, such as animal proteins, for the manufacture of transgenic products.

One example of the plant seeds to which a platform of the present disclosure is applied is a soybean plant. Among the proteins accumulated in soybean seeds, the majority is storage protein. There are two soybean seed storage proteins: glycinin (11S globulin) and beta-conglycinin (7S globulin). Together, they comprise about 70% to 80% of the total seed protein, and 25% to 35% of the dry weight of the seed. Both of these proteins are made up of multiple isoforms derived from gene families.

Glycinin (11S globulin) and β-conglycinin (7S globulin) are the most important soybean proteins. The 2S fraction is found in a lesser amount and consists of Bowman-Birk and Kunitz trypsin inhibitors, cytochrome c, and α-conglycinin.

Glycinin is a major soybean seed storage protein with a molecular weight of about 360 kDa. It is a hexamer composed of the various combinations of five major subunits identified as G1, G2, G3, G4 and G5. As other legumin-like globulins, glycinin consists of one basic and one acidic polypeptide, which are linked by a single disulfide bond, except for the acidic polypeptide A4. The molecular masses of the basic polypeptides are around 20 kDa, while those of the acidic polypeptides are around 38 kDa. At ambient temperatures and pH 7.6 (I=0.5 M), glycinin forms hexamers (11S) with molecular masses around 300-360 kDa.

Beta-conglycinin is a heterogeneous glycoprotein with a molecular weight ranging from 150 and 240 kDa. It is composed of varying combinations of three highly negatively charged subunits identified as α, α′ and β. In wild-type seeds, β-conglycinin comprises about 15-20% of the total soybean protein. β-Conglycinin is a trimeric glycoprotein (7S) consisting of three types of subunits, α′ (57-72 kDa), α (57-68 kDa) and β (45-52 kDa) in different combinations. As for other 7S globulins, the subunits are associated via hydrophobic and hydrogen bonded interactions without the contribution of disulfide bonds. At pH 5 and higher, β-conglycinin is found as a trimer (I=0.5 M), whereas it predominantly exists as a hexamer at ionic strength lower than 0.1 M. At pH values 2-5, β-conglycinin reversibly dissociates into a 2-3S and 5-6S fraction at ionic strength lower than 0.1 M.

The seeds of many plant species contain storage proteins. These proteins have been classified on the basis of their size and solubility (Higgins, T. J. (1984) Ann. Rev. Plant Physiol. 35:191-221). While not every class is found in every species, the seeds of most plant species contain proteins from more than one class. Proteins within a particular solubility or size class are generally more structurally related to members of the same class in other species than to members of a different class within the same species. In many species, the seed proteins of a given class are often encoded by multigene families, sometimes of such complexity that the families can be divided into subclasses based on sequence homology.

Plant storage proteins, especially those processed through the secretory pathway, generally undergo multiple post-translational processing steps including folding, assembly, intracellular sorting, and proteolytic processing, prior to final deposition (Muntz et al. (1993) Proc. Phytochem. Soc. Eur. 35: 128-146; Muntz (1998) Plant Mol. Biol. 38: 77-99; Herman and Larkins (1999) Plant Cell 11: 601-613). Accumulation and deposition of the proteins is accomplished by compartmentalization in specialized vacuoles termed protein storage vacuoles and or protein bodies (Hara-Nishimura et al. (1995) J. Plant Physiol. 145: 632-640; Mintz (1998) Plant Molec. Biol. 38: 77-99; Herman and Larkins (1999) Plant Cell 11: 601-613).

The proteolytic processing steps of protein deposition in vacuoles include specific polypeptide cleavage steps accomplished by proteases localized to the storage vacuole (Bassham et al. (2000) Curr. Opin. Cell Biol. 12: 491-495). Storage proteins that accumulate in vacuoles have therefore co-evolved with the environment of the storage vacuole, such that only a select few protease sites exist or are accessible to these proteases (Hara-Nishimura et al. (1987) Plant Physiol. 85: 440-445; D'Hondt et al., (1993) J. Biol. Chem. 268: 10884-10891; Hara-Nishimura et al. (1993) Plant Cell 5: 1651-1659; Hara-Nishimura et al. (1995) J. Plant Physiol. 145: 632-640).

In some embodiments, seed proteome rebalancing is employed to increase expression of a desired protein product by genomically disrupting an endogenous gene that encodes a storage protein. In some embodiments, storage proteins in target plants can be identified by functional, structural, and/or sequence homologies and can be utilized for seed proteome rebalancing.

In some embodiments, the plant seed is a soybean seed. Non-limiting examples of plants for use of their seeds in the present disclosure include Vicia genus, Phaseolus genus, Vigna genus, Cicer genus, Pisum genus, Lathyrus genus, Lens genus, lablab genus, Glycine genus, Psophocarpus genus, Cajanus genus, Mucuna genus, Cyamopsis genus, Canavalia genus Macrotyloma genus Lupinus genus and Archis genus. Vicia genus includes Vicia faba (broad beans, known in the US as fava beans). Phaseolus genus includes Phaseolus acutifolius (tepary bean), Phaseolus coccineus (runner bean), Phaseolus lunatus (lima bean), Phaseolus vulgaris (common bean; includes the pinto bean, kidney bean, black bean, Appaloosa bean as well as green beans, and many others), and Phaseolus polyanthus (a.k.a. P. dumosus). Vigna genus includes Vigna aconitifolia (moth bean), Vigna angularis (adzuki bean), Vigna mungo (urad bean), Vigna radiata (mung bean), Vigna subterranea (Bambara bean or ground-bean), Vigna umbellata (ricebean), and Vigna unguiculata (cowpea; also includes the black-eyed pea, yardlong bean and others). Cicer genus includes Cicer arietinum (chickpea or garbanzo bean). Pisum genus includes Pisum sativum (pea). Lathyrus genus includes Lathyrus sativus (Indian pea), and Lathyrus tuberosus (tuberous pea). Lens genus includes Lens culinaris (lentil). Lablab genus includes Lablab purpureus (hyacinth bean). Glycine genus include Glycine max (soybean). Psophocarpus genus includes Psophocarpus tetragonolobus (winged bean) and Psophocarpus tetragonolobus (winged bean). Cajanus genus includes Cajanus cajan (pigeon pea). Mucuna genus includes Mucuna pruriens (velvet bean). Cyamopsis genus includes Cyamopsis tetragonoloba (guar). Canavalia genus includes Canavalia ensiformis (jack bean) and Canavalia gladiata (sword bean). Macrotyloma genus includes Macrotyloma uniflorum (horse gram). Lupinus (lupin) genus includes Lupinus mutabilis (tarwi) and Lupinus albus (lupini bean). Arachis genus includes Arachis hypogaea (peanut).

In some embodiments, non-limiting examples of plants for use of their seeds in the present disclosure include Avena genus, Zea genus, and Oryza genus. Avena genus includes cultivated oats such as Avena sativa, Avena abyssinica, Avena byzantina, Avena nuda, and Avena strigosa. Zea genus includes Zea mays. Oryza genus includes a food crop rice such as Oryza sativa and Oryza glaberrima.

### Amino Acid Rebalancing (Seed Proteome Rebalancing)

Seeds have evolved to store triglycerides, non-structural reserve carbohydrates and protein at maximum density within storage cells, leaving little cellular space to add additional products resulting from transgene expression. Within its developmental program, the seed exhibits a limited degree of storage substance plasticity, and rebalances storage protein content via nutrient availability, such as sulphur (Beach et al., 1985 Nucleic Acids Res. 13, 999-1013; Hirai et al., 1995 Plant Cell Physiol. 36, 1331-1339; Hagan et al., 2003 Plant J. 34, 1-11; Tabe et al., 2002 Curr. Opin. Plant Biol. 5, 212-217).

In some embodiments, one strategy to enhance foreign protein production involves exchanging the capacity to produce intrinsic proteins for the capacity to produce a high level of foreign proteins. In some embodiments, the collateral proteome rebalancing that occurs with the suppression of endogenous proteins in soybean can be exploited to produce an enhanced level of foreign proteins.

The present disclosure teaches use of the protein synthesis capacity of a high-protein seed by redirecting a significant part of the protein synthesis capacity from the production of intrinsic seed proteins to the synthesis of foreign protein(s). Large-scale alteration of the proteome can be produced by the introduction of gene-editing systems that suppress and/or knock out one or more of the seed proteins.

For beans such as soybeans, like many other seeds (Her-man and Larkins, 1999 Plant Cell, 11, 601-661), the two major storage proteins, glycinin (11S legumin type) and conglycinin (7S vicilin type), dominate the proteome. The soybean seed proteome also includes many moderately abundant proteins that are bioactive and allergenic, such as the Kunitz and Bowman-Birk trypsin inhibitors, lectin, P34 allergen, sucrose binding protein, urease, oleosins (Herman and Burks, 2011 Curr. Opin. Biotechnol. 22, 224-230) and several thousand low abundance proteins, including enzymes that mediate metabolism, synthesize storage substances, and create the structural framework of the cell. The specific mix of proteins and each protein's abundance within the proteome determines the total amino acid composition trait.

In some embodiments, suppression of one seed protein can lead to a compensation by an increase in the production of other seed proteins, termed “compensation” or “rebalancing.” In some embodiments, this seed proteome rebalancing is presented in the present disclosure with plant lines having a deficiency in seed proteins including, but not limited to β-conglycinin α, β-conglycinin α′, β-conglycinin β, Cysteine Protease, Glycinin 1, Glycinin 2, Glycinin 3, Glycinin 4, Glycinin 5, Glycinin 7, Kunitz-type Trypsin inhibitor, and lectin by gene-editing techniques described herein. In some embodiments, seed proteome rebalancing is presented in the present disclosure with plant lines having a deficiency in proteins for example, listed in Tables 3-1, 3-2, and 4, in plants of interest by gene-editing techniques described herein. In some embodiments, a plant (or specific plant part, such as a bean) has reduced expression of a protein selected from the group consisting of: β-conglycinin α, β-conglycinin α′, β-conglycinin β, Cysteine Protease, Glycinin 1, Glycinin 2, Glycinin 3, Glycinin 4, Glycinin 5, Glycinin 7, Kunitz-type Trypsin inhibitor, and lectin. In some embodiments, a CRISPR system is employed to effectuate a genomic disruption of at least a portion of a gene thereby reducing or eliminating expression of a protein encoded by the portion of the gene.

For the seed proteome rebalancing, a target protein for genetic deficiency is determined by an amino acid rebalancing method described herein.

Provided is an amino acid rebalancing platform (also interchangeably used as an amino acid bottleneck identification). An amino acid rebalancing platform can be used to identify which of most abundant proteins in seeds have an amino acid profile that best matches the profile of a target protein or recombinant protein of interest in a view of compensating each other when the amino acid rebalancing is demanded. In some embodiments, one or more endogenous proteins in plant seeds are selected to be reduced or knocked out (or optionally knocked down) in order to trigger the amino acid rebalancing in a plant seed and make enough metabolites available for the synthesis of the proteins of interest described herein. In some embodiments, seeds are malleable structures which are able to redirect metabolic resources from certain protein types to others and present the ability to maintain an overall final amino acid content.

In some embodiments, targets for gene editing are selected from their predicted demand on the seed proteome, in relationship to the demands on the seed imposed by recombinant proteins. For example, the ratio of Proline to other amino acids can be determined to have higher in kappa-casein than in soybean seeds reported in Kovalenko et al 2006, when compared amino acid compositions as described herein. From the most prominent seed storage proteins based on RNAseq (Gunadi et al. 2016), the highest percentage AA utilization of Proline is in the proteins coded by Glyma.13G123500, and Glyma.10G037100, at 7.16% Proline and 6.75% Proline, respectively as shown in Table 69. Without these two genes, demands on the seed's free amino acid pool more Proline are available for the recombinant protein and accumulation of the recombinant protein increases.

In some embodiments, a method comprises: (a) determining amino acid demands for a recombinant protein; (b) calculating the difference (A) between the most required amino acids and the provided balance in soybean; and (c) selecting a soy protein that that will release most of the amino acids identified in steps (a) and (b).

In some embodiments, the genetic deficiency results in an amount of a specific seed-associated protein (e.g. seed storage protein) that is less than about 1%, about 2%, about 5%, about 10%, about 25%, about 50%, about 75%, or about 85% of the amount of the endogenous seed storage protein that is normally present in a WT soybean seed. In other embodiments, one or more the seed-associated proteins listed in Tables 69-71 are suppressed, repressed, reduced, and/or eliminated by a CRISPR-Cas system.

In other embodiments, the target sequence for genetic deficiency is a nucleic acid sequence encoding a seed protein. In further embodiments, the seed protein is chosen based on its predicted free amino acid demand on the seed proteome when compared to that of the recombinant protein of interest as described in Example 1.

In some embodiments, provided herein is an amino acid rebalancing based method for enhanced expression of a protein of interest in a plant seed, comprising: a) calculating a % amino acid composition of the protein of interest; b) calculating the endogenous amino acid balance provided by the plant seed; c) identifying an amino acid rebalancing target profile, by calculating the % difference (A %) between the amino acids required to produce the protein of interest and the endogenous amino acid balance provided by the plant seed; d) ranking each amino acid from the amino acid rebalancing target profile by A %; e) identifying an endogenous seed storage protein comprising an amino acid that ranks in the top 5 Δ % from the amino acid rebalancing target profile ranked in (d); f) disrupting expression of an endogenous target gene encoding the seed storage protein identified in (e); and g) expressing the protein of interest in the plant cell.

In other embodiments, provided herein is an amino acid rebalancing based method for enhanced expression of a protein of interest in a plant seed, comprising: a) calculating a % amino acid composition of the protein of interest; b) calculating the endogenous amino acid balance of each highly expressed protein based on % expression level of seed proteins in the seed; c) identifying an amino acid rebalancing target profile, by calculating the % difference (Δ %) between the amino acid composition of step (a) and the endogenous amino acid balance of step (b); d) identifying endogenous seed storage proteins with a positive value on the amino acid rebalancing target profile of step (c); e) disrupting expression of at least one endogenous target gene encoding the seed storage protein identified in step (d); and f) expressing the protein of interest in the plant cell, when the protein is considered to be expressed at least 0.5%, at least 1%, at least 2%, at least 3%, at least 4%, at least 5%, at least 6%, at least 7%, at least 8%, at least 9%, or at least 10% in the seed.

### Enhancing Production of Proteins of Interest in Seeds Using Amino Acid Rebalancing

The present disclosure teaches that suppression of the seed protein listed in Tables 3-1, 3-2 and 4 by sequence mediated gene silencing is compensated for by an increased abundance of heterologous proteins of interest encoded by nucleic acid sequences listed in Table 9.

In some embodiments, seed storage protein suppression is achieved using CRISPPR/Cas technology as described above and in Examples 1 and 2. This method results in the complete silencing of target seed storage protein. A fraction of the increased production of heterologous proteins of interest can be retained in the form of its precursor and be sequestered in Protein Bodies (PBs) directly assembled within the endoplasmic reticulum (ER) or Protein Storage vacuoles (VSRs). Accumulation of proteins in a protein body, instead of the PSV, demonstrates two important points: 1) that ER-derived PBs can be induced and accumulate proteins in soybean seeds and, 2) that suppression of an endogenous storage protein results in the increased accumulation of another storage protein to compensate for mass loss. This phenomenon maintains the overall protein content of the soybean seed to 40%, and is termed ‘rebalancing’.

In some embodiments, plant lines having a deficiency of both Glycinin 4 and Glycinin 5 are prepared, as described in Example 1. In these seeds that are genetically deficient in both Glycinin 4 and Glycinin 5, the protein loss is compensated by the production of other foreign proteins of interest. The changes in protein production can be seen by protein detection methods described in Example 1 when compared seed protein extracts of WT to that of the transgenic seeds. The removal of the seed proteins Glycinin 4 and Glycinin 5 in the transgenic line can result in compensation by other foreign kappa-casein proteins by amino acid rebalancing due to the highest demand for Proline, which was calculated and designed for enhanced expression of the protein of interest that can compensate for the lack of protein the most.

In other embodiments, the present disclosure teaches that a deficiency and/or suppression of one or more seed storage proteins disclosed in Table 3 such as β-conglycinin α, β-conglycinin α′, β-conglycinin J, Cysteine Protease, Glycinin 1, Glycinin 2, Glycinin 3, Glycinin 4, Glycinin 5, Glycinin 7, Kunitz-type Trypsin inhibitor, and Lectin can be introduced using an amino acid rebalancing platform, as described in Example 1. In these seeds that are genetically deficient in one or more seed storage proteins disclosed in Table 3, the protein loss triggers collateral changes of the seed proteome that provides opportunity of increased production of other foreign proteins of interest, which compensate for the loss of amino acids. The changes in protein production can be seen by protein detection methods described in Example 24.

The removal of the seed proteins such as β-conglycinin α, β-conglycinin α′, β-conglycinin β, Glycinin 1, Glycinin 4, and Kunitz-Trypsin inhibitor as described in Example 1 can be compensated by expression of other foreign proteins such as κ-Casein, Ovalbumin, Vitellogenin-2 and a chicken fibroblast growth factor.

Provided herein are seeds that possess an intrinsic biology that may be exploited as the foundation of a protein production platform by having a foreign protein share in the rebalancing process and by accumulating the foreign protein in a stable population of PBs. Together this is the basis of developing dicot seeds as a protein production platform. In some embodiments, one (or more) seed storage proteins is reduced as discussed above, and a desired heterologous protein is produced in the seed. In some embodiments, any suitable promoter is operably linked to the sequence encoding the heterologous protein. In another embodiment, the sequence encoding the heterologous protein is operatively linked to a seed-specific promoter taught in the present disclosure. The promoter can be, for example, a tissue specific promoter described herein.

In some embodiments, increased expression of the heterologous protein can occur when its gene sequence is operably linked to a promoter of the gene that encodes a protein that is upregulated in response to the above-described genetic deficiency in a soybean seed. For each specific protein that is removed from a seed, another protein (or proteins) may be produced in its place. By using the gene regulatory region (such as the promoter, terminator, and optionally other regions) of this “compensating” protein to drive the expression of the heterologous gene of interest, one can obtain an even higher level of protein production in the seed. Accordingly, in some embodiments, the seed protein that is suppressed is one or more proteins listed in Tables 3-1, 3-2 and 4, while the expression of the heterologous protein is controlled by at least a portion of the regulatory region of genes listed in Table 9. In some embodiments, this regulatory region is located upstream of the heterologous sequence encoding protein of interest. In some embodiments, the regulatory region is downstream or 3′ end of the heterologous sequence. In some embodiments, the regulatory region comprises the promoters listed in Table 9. In some embodiments, the regulatory region is the upstream regulatory region, which can include, for example, the promoter and/or the 5′ UTR. In other embodiments, the regulatory region also includes the 3′ regulatory region. In some embodiments, the upstream regulatory region includes a signal peptide sequence of genes listed in Table 9.

In some embodiments, the seed protein that is suppressed is Glycinin 4 and Glycinin 5, while the heterologous protein such as protein of interest is controlled by at least a portion of the regulatory regions (5′, 3′, and/or signal peptide sequence) from genes listed in Table 1.

In some embodiments, the seed protein that is suppressed is β-conglycinin α, β-conglycinin α′, β-conglycinin β, Glycinin 1, Glycinin 4, and Kunitz-Trypsin inhibitor, while the heterologous protein such as protein of interest is controlled by at least a portion of the regulatory regions (5′, 3′, and/or signal peptide sequence) from genes listed in Table 9.

In one example, by placing the heterologous expression cassette under a seed-specific promoter such as regulatory region of one of Glycinin genes, the expression of the heterologous transcript will mimic that of glycinin gene expression and regulation and thereby likely participate in nutrient allocation that involves upregulation of glycinin genes. Also, expression of the heterologous protein of interest will be further enhanced by placing another gene-editing cassette aimed for knocking out the endogenous genes coding for seed storage proteins, for example, β-conglycinin α, β-conglycinin α′, β-conglycinin β, Glycinin 1, Glycinin 4, Glycinin 5, and Kunitz-Trypsin inhibitor. Soybeans expressing one or more transgenes and suppressing endogenous seed protein show increased accumulation of the heterologous foreign protein in the seed. Thus, the enhancement of foreign protein accumulation in the seeds demonstrates that mimicking the allele of the gene participating in protein rebalancing can result in a large increase in accumulation of the heterologous protein of interest such as x-Casein, Ovalbumin, Vitellogenin-2 and a chicken fibroblast growth factor as examples.

In another example, by placing the heterologous protein expression cassette under a seed-specific promoter such as regulatory region of one of β-conglycinin genes, the expression of the heterologous transcript will mimic that of β-conglycinin gene expression and regulation and thereby likely participate in nutrient allocation that involves upregulation of β-conglycinin genes. Also, expression of the heterologous protein of interest will be further enhanced by placing another gene-editing cassette aimed for knocking out the endogenous genes coding for seed storage proteins, for example, β-conglycinin α, β-conglycinin α′, β-conglycinin J, Glycinin 1, Glycinin 4, and Kunitz-Trypsin inhibitor. Soybeans expressing one or more transgenes and suppressing endogenous seed protein show increased accumulation of the heterologous foreign protein in the seed. Thus, the enhancement of foreign protein accumulation in the seeds demonstrates that mimicking the allele of the gene participating in protein rebalancing can result in a large increase in accumulation of the heterologous protein of interest such as κ-Casein, Ovalbumin, Vitellogenin-2, and a chicken fibroblast growth factor as examples.

In some embodiments, the platform described herein enhances total protein accumulation by altering certain metabolic pathways such as nitrogen fixation or proteolysis activities. In certain embodiments, the platform focus on enhancing recombinant protein production. Approaches adopted to increase recombinant protein expression include but not limited to: i) using protein storage unit signal peptide or ER retention signal to facilitate protein accumulation; and ii) placing multiple expression cassettes to insert different optimized sequences encoding the same protein.

In some embodiments, the proteome rebalancing in crop seeds is achieved through amino acid rebalancing. In other embodiments, precise and efficient amino acid rebalancing can be achieved by gene-editing techniques taught in the present disclosure. In specific embodiments, both gene-editing cassette and multiple expression cassettes are introduced in the same binary vector, in order to maximize the recombinant protein production.

In some embodiments, an increased amount of a protein of interest can be produced in the seed. For example, the heterologous protein can be expressed from about 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, or more of the total soluble protein in the seed.

In some embodiments, the dry weight of the heterologous protein can be expressed from about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50% or more of the total dry weight of the seed.

In some embodiments, the heterologous protein can be produced, for example, at an amount of about 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 100 mg, 150 mg, 200 mg, 250 mg, 300 mg, 350 mg or more protein per seed.

In some embodiments, the amount of heterologous protein produced can be measured on a per plant basis. The heterologous protein can be produced, for example, at an amount of about 0.1 g, 0.2 g, 0.3 g, 0.4 g, 0.5 g, 0.6 g, 0.7 g, 0.8 g, 0.9 g, 1 g, 2 g, 3 g, 4 g, 5 g, 6 g, 7 g, 8 g, 9 g, 10 g, 11 g, 12 g, 13 g, 14 g, 15 g, 16 g, 17 g, 18 g, 19 g, 20 g, 25 g, 30 g, 35 g, 40 g, 45 g, 50 g, 75 g, 100 g or more of heterologous protein per plant.

In some embodiments, the heterologous protein can be produced, for example, at an amount of about 0.5 pounds, 1 pound, 2 pounds, 3 pounds, 4 pounds, 5 pounds, 6 pounds, 7 pounds, 8 pounds, 9 pounds, 10 pounds, 15 pounds, 20 pounds, 25 pounds, 30 pounds, 40 pounds, 50 pounds, 75 pounds, 100 pounds, 200 pounds, 300 pounds, 400 pounds, 500 pounds, 600 pounds, 700 pounds, 800 pounds, 900 pounds, 1000 pounds, 1500 pounds, 2000 pounds, 3000 pounds, 4000 pounds, 5000 pounds, or more pounds per acre per season. The actual yield can depend on many parameters such as plants per acre, plant variety, soil quality, cultivating practices, plant stress, and also the level of purity of the heterologous protein to be produced.

### Suppression of Seed Storage Protein

Expression of pivotal storage proteins can be suppressed to a substantial portion of the plant's endogenous seed storage protein content or even be completely abolished by genetic engineering technologies taught in the present disclosure, such as gene-editing CRISPR-Cas system.

In some embodiments, a plant can be deficient in one or two more seed storage proteins that comprising globulin storage proteins (including 11S globulins and 7S globulins), 2S albumin storage proteins, prolamin storage proteins, or other seed storage proteins. In some embodiments, a plant of the present disclosure can be deficient in one or two more globulin storage proteins comprising glycinin (11S globulin) and conglycinin (7S globulins). Structures and biosynthesis of Seed Storage Proteins are known in the art, for example, shewry et al. (1995) Plant Cell 7:945-956, which is expressly incorporated herein by reference in its entirety.

Non-limiting example of the seed storage protein is Glycinin (11S), beta-conglycinin (7S) proteins, Cupin protein, lectin protein, trypsin and protease inhibitor proteins. In some embodiments, the present disclosure teaches suppression and repression of the seed storage protein including Glycinin (11S), beta-conglycinin (7S) proteins, Cupin protein, lectin protein, trypsin and protease inhibitor proteins using genetic engineering techniques described herein.

In an embodiment, genetic manipulation to create a deficiency of a seed storage protein can be obtained by methods such as gene editing, co-suppression, antisense, RNAi, or other methods. U.S. Pat. No. 5,190,931 describes exemplary methods of the use of an antisense construct to downregulate a gene. U.S. Pat. No. 5,231,020 describes exemplary methods of the use of a sense nucleic acid construct to downregulate a gene. Genetic inhibition the expression of a gene product by use of double-stranded mRNA is disclosed in U.S. Pat. No. 6,506,559.

“Gene suppression” refers to any of the well-known methods for reducing the levels of gene transcription to mRNA and/or subsequent translation of the mRNA. Gene suppression is also intended to mean the reduction of protein expression from a gene or a coding sequence including posttranscriptional gene suppression and transcriptional suppression. Posttranscriptional gene suppression is mediated by the homology between of all or a part of a mRNA transcribed from a gene or coding sequence targeted for suppression and the corresponding double stranded RNA used for suppression, and refers to the substantial and measurable reduction of the amount of available mRNA available in the cell for binding by ribosomes. The transcribed RNA can be in the sense orientation to effect what is called co-suppression, in the anti-sense orientation to effect what is called anti-sense suppression, or in both orientations producing a dsRNA to effect what is called RNA interference (RNAi).

Transcriptional suppression is mediated by the presence in the cell of a dsRNA gene suppression agent exhibiting substantial sequence identity to a promoter DNA sequence or the complement thereof to effect what is referred to as promoter trans suppression. Gene suppression may be effective against a native plant gene associated with a trait, e.g., to provide plants with reduced levels of a protein encoded by the native gene or with enhanced or reduced levels of an affected metabolite. Gene suppression can also be effective against target genes in plant pests that may ingest or contact plant material containing gene suppression agents, specifically designed to inhibit or suppress the expression of one or more homologous or complementary sequences in the cells of the pest. Post-transcriptional gene suppression by anti-sense or sense-oriented RNA to regulate gene expression in plant cells is disclosed in U.S. Pat. Nos. 5,107,065, 5,759,829, 5,283,184, and 5,231,020. The use of dsRNA to suppress genes in plants is disclosed in WO 99/53050, WO 99/49029, U.S. Patent Application Publication No. 2003/0175965, and 2003/0061626, U.S. patent application Ser. No. 10/465,800 (abandoned), and U.S. Pat. Nos. 6,506,559, and 6,326,193.

A method of post transcriptional gene suppression in plants employs both sense-oriented and anti-sense-oriented, transcribed RNA which is stabilized, e.g., as a hairpin and stem and loop structure. A preferred DNA construct for effecting post transcriptional gene suppression is one in which a first segment encodes an RNA exhibiting an anti-sense orientation exhibiting substantial identity to a segment of a gene targeted for suppression, which is linked to a second segment in sense orientation encoding an RNA exhibiting substantial complementarity to the first segment. Such a construct forms a stem and loop structure by hybridization of the first segment with the second segment and a loop structure from the nucleotide sequences linking the two segments (see WO94/01550, WO98/05770, US 2002/0048814, and US 2003/0018993).

In some embodiments, methods for achieving a deficiency of a seed storage protein are not transcriptional gene suppression or post transcriptional gene suppression via RNAi.

In some embodiments, genetic manipulation to create a deficiency of a seed storage protein can be obtained by methods for engineering plant genomes using CRISPR/CAS systems, which are well known in the art, such as U.S. Patent Application Publication Nos. 2018/0195089, US2016/0102322, US2015/0167000, US2014/0273235, US2019/0093107, US2017/0114351, and U.S. Pat. Nos. 9,790,490 and 9,879,283, each of which is expressly incorporated herein by reference in their entirety.

In other embodiments, a deficiency of a particular seed storage protein, or seed storage proteins in the aggregate, can also be attained by conventional breeding methods followed by screening for a low level of one or more seed proteins. A deficiency of a seed storage protein can also be attained by natural mutations or induced mutations, followed by screening methods to identify those plants having a low level of one or more seed storage proteins. In another embodiment, a plant having a deficiency of a seed storage protein can be obtained, for example, from a publicly available seed bank or seed repository.

While RNAi downregulates the gene expression, CRISPR/Cas gene-editing system knocks out the target gene. The present disclosure teaches transgenic seeds, in which one or more gene encoding seed storage proteins are knocked out by CRISPR/Cas systems, would free up more amino acids for production of heterologous proteins of interest.

As described above, RNAi knockdown is at transcriptional and/or post transcriptional level, which are reversible, while CRISPR/Cas9 mediated knockout can permanently remove a target gene, thereby suppressing and/or triggering a deficiency of protein encoded by said target gene, such as seed storage protein. In some embodiments, CRISPR/Cas systems can create mutant plants without foreign gene insertion in the progeny.

The present disclosure teaches that RNAi can only regulate gene expression, while CRISPR/Cas system can induce a variety of mutations such as frameshift mutation, truncated or loss-of-function protein, or even gene insertion as well.

The present disclosure teaches that RNAi silencing method suffers from high off-target effects, which contains two types: sequence-independent and sequence-dependent. For instance, siRNAs trigger an interferon activated pathway in certain cell types in a sequence-independent manner, resulting in increased expression of interferon-regulated genes (Ui-Tei, 2013, Front. Genet. 4:107 and Sledz et al, 2003, Nature Cell Bilogy 5(9):834) The CRISPR system has advanced rapidly with the help of efficient design tools that enable minimal off-target effects. A recent study comparing these two technologies revealed that CRISPR method is far less susceptible to systematic off-target effects (Smith et al, 2017, PLoS Biology 15(11), e2003213).

### Gene Editing

As used herein, the term “gene editing system” refers to a system comprising one or more DNA-binding domains or components and one or more DNA-modifying domains or components, or isolated nucleic acids, e.g., one or more vectors, encoding said DNA-binding and DNA-modifying domains or components. Gene editing systems are used for modifying the nucleic acid of a target gene and/or for modulating the expression of a target gene. In known gene editing systems, for example, the one or more DNA-binding domains or components are associated with the one or more DNA-modifying domains or components, such that the one or more DNA-binding domains target the one or more DNA-modifying domains or components to a specific nucleic acid site. Methods and compositions for enhancing gene editing is well known in the art. See example, U.S. Patent Application Publication No. 2018/0245065, which is incorporated by reference in its entirety.

Certain gene editing systems are known in the art, and include but are not limited to, zinc finger nucleases, transcription activator-like effector nucleases (TALENs); clustered regularly interspaced short palindromic repeats (CRISPR)/Cas systems, meganuclease systems, and viral vector-mediated gene editing.

In some embodiments, the present disclosure teaches methods for gene editing/cloning utilizing DNA nucleases. CRISPR complexes, transcription activator-like effector nucleases (TALENs), zinc finger nucleases (ZFNs), and FokI restriction enzymes, which are some of the sequence-specific nucleases that have been used as gene editing tools. These enzymes are able to target their nuclease activities to desired target loci through interactions with guide regions engineered to recognize sequences of interest. In some embodiments, the present disclosure teaches CRISPR-based gene editing methods.

### (i) CRISPR Systems

CRISPR (Clustered Regularly Interspaced Short Palindromic Repeats) and CRISPR-associated (cas) endonucleases were originally discovered as adaptive immunity systems evolved by bacteria and archaea to protect against viral and plasmid invasion. Naturally occurring CRISPR/Cas systems in bacteria are composed of one or more Cas genes and one or more CRISPR arrays consisting of short palindromic repeats of base sequences separated by genome-targeting sequences acquired from previously encountered viruses and plasmids (called spacers). (Wiedenheft, B., et. al. Nature. 2012; 482:331; Bhaya, D., et. al., Annu. Rev. Genet. 2011; 45:231; and Terms, M. P. et. al., Curr. Opin. Microbiol. 2011; 14:321). Bacteria and archaea possessing one or more CRISPR loci respond to viral or plasmid challenge by integrating short fragments of foreign sequence (protospacers) into the host chromosome at the proximal end of the CRISPR array. Transcription of CRISPR loci generates a library of CRISPR-derived RNAs (crRNAs) containing sequences complementary to previously encountered invading nucleic acids (Haurwitz, R. E., et. al., Science. 2012:329; 1355; Gesner, E. M., et. al., Nat. Struct. Mol. Biol. 2001:18; 688; Jinek, M., et. al., Science. 2012:337; 816-21). Target recognition by crRNAs occurs through complementary base pairing with target DNA, which directs cleavage of foreign sequences by means of Cas proteins. (Jinek et. al. 2012 “A Programmable dual-RNA-guided DNA endonuclease in adaptive bacterial immunity.” Science. 2012:337; 816-821).

There are at least five main CRISPR system types (Type I, II, III, IV and V) and at least 16 distinct subtypes (Makarova, K. S., et al., Nat Rev Microbiol. 2015. Nat. Rev. Microbiol. 13, 722-736). CRISPR systems are also classified based on their effector proteins. Class 1 systems possess multi-subunit crRNA-effector complexes, whereas in Class 2 systems all functions of the effector complex are carried out by a single protein (e.g., Cas9 or Cpf1). In some embodiments, the present disclosure provides using type II and/or type V single-subunit effector systems.

As these naturally occur in many different types of bacteria, the exact arrangements of the CRISPR and structure, function and number of Cas genes and their product differ somewhat from species to species. Haft et al. (2005) PLoS Comput. Biol. 1: e60; Kunin et al. (2007) Genome Biol. 8: R61; Mojica et al. (2005) J. Mol. Evol. 60: 174-182; Bolotin et al. (2005) Microbiol. 151: 2551-2561; Pourcel et al. (2005) Microbiol. 151: 653-663; and Stem et al. (2010) Trends. Genet. 28: 335-340. For example, the Cse (Cas subtype, E. coli) proteins (e.g., CasA) form a functional complex, Cascade, that processes CRISPR RNA transcripts into spacer-repeat units that Cascade retains. Brouns et al. (2008) Science 321: 960-964. In other prokaryotes, Cas6 processes the CRISPR transcript. The CRISPR-based phage inactivation in E. coli requires Cascade and Cas3, but not Cas1 or Cas2. The Cmr (Cas RAMP module) proteins in Pyrococcus furiosus and other prokaryotes form a functional complex with small CRISPR RNAs that recognizes and cleaves complementary target RNAs. A simpler CRISPR system relies on the protein Cas9, which is a nuclease with two active cutting sites, one for each strand of the double helix. Combining Cas9 and modified CRISPR locus RNA can be used in a system for gene editing. Pennisi (2013) Science 341: 833-836.

### (ii) CRISPR/Cas

In some embodiments, the present disclosure provides methods of gene editing using a Type II CRISPR system. Type II systems rely on a i) single endonuclease protein, ii) a transactiving crRNA (tracrRNA), and iii) a crRNA where a ˜20-nucleotide (nt) portion of the 5′ end of crRNA is complementary to a target nucleic acid. The region of a CRISPR crRNA strand that is complementary to its target DNA protospacer is hereby referred to as “guide sequence.”

In some embodiments, the tracrRNA and crRNA components of a Type II system can be replaced by a single guide RNA (sgRNA), also known as a guide RNA (gRNA). The sgRNA can include, for example, a nucleotide sequence that comprises an at least 12-20 nucleotide sequence complementary to the target DNA sequence (guide sequence) and can include a common scaffold RNA sequence at its 3′ end. As used herein, “a common scaffold RNA” refers to any RNA sequence that mimics the tracrRNA sequence or any RNA sequences that function as a tracrRNA.

Cas9 endonucleases produce blunt end DNA breaks, and are recruited to target DNA by a combination of a crRNA and a tracrRNA oligos, which tether the endonuclease via complementary hybridization of the RNA CRISPR complex.

In some embodiments, DNA recognition by the crRNA/endonuclease complex requires additional complementary base-pairing with a protospacer adjacent motif (PAM) (e.g., 5′-NGG-3′) located in a 3′ portion of the target DNA, downstream from the target protospacer. (Jinek, M., e. al., Science. 2012:337; 816-821). In some embodiments, the PAM motif recognized by a Cas varies for different Cas proteins. In some embodiments, a Cas is a Cas9.

In some embodiments the Cas9 disclosed herein can be any variant derived or isolated from any source. In other embodiments, the Cas9 peptide of the present disclosure can include one or more of the mutations described in the literature, including but not limited to the functional mutations described in: Fonfara et al. Nucleic Acids Res. 2014 February; 42(4):2577-90; Nishimasu H. et al. Cell. 2014 Feb. 27; 156(5):935-49; Jinek M. et al. Science. 2012 337:816-21; and Jinek M. et al. Science. 2014 Mar. 14; 343(6176); see also U.S. patent application Ser. No. 13/842,859, filed Mar. 15, 2013, which is hereby incorporated by reference; further, see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641, which are all hereby incorporated by reference. Thus, in some embodiments, the systems and methods disclosed herein can be used with the wild type Cas9 protein having double-stranded nuclease activity, Cas9 mutants that act as single stranded nickases, or other mutants with modified nuclease activity.

According to the present disclosure, Cas9 molecules of, derived from, or based on the Cas9 proteins of a variety of species can be used in the methods and compositions described herein. For example, Cas9 molecules of, derived from, or based on, e.g., S. pyogenes, S. thermophilus, Staphylococcus aureus and/or Neisseria meningitidis Cas9 molecules, can be used in the systems, methods and compositions described herein. Additional Cas9 species include: Acidovorax avenae, Actinobacillus pleuropneumoniae, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhiz obium sp., Brevibacillus latemsporus, Campylobacter coli, Campylobacter jejuni, Campylobacter lad, Candidatus Puniceispirillum, Clostridiu cellulolyticum, Clostridium perfringens, Corynebacterium accolens, Corynebacterium diphtheria, Corynebacterium matruchotii, Dinoroseobacter sliibae, Eubacterium dolichum, gamma proteobacterium, Gluconacetobacler diazotrophicus, Haemophilus parainfluenzae, Haemophilus sputorum, Helicobacter canadensis, Helicobacter cinaedi, Helicobacter mustelae, Ilyobacler polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica. Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tislrella mobilis, Treponema sp., or Verminephrobacter eiseniae.

In some embodiments, the present disclosure teaches the use of tools for genome editing techniques in plants such as crops and methods of gene editing using CRISPR-associated (cas) endonucleases including SpyCas9, SaCas9, St1Cas9. These powerful tools for genome editing, which can be applied to plant genome editing are well known in the art. See example, Song et al. (2016), CRISPR/Cas9: A powerful tool for crop genome editing, The Crop Journal 4:75-82, Mali et al. (2013) RNA-guided human genome engineering via cas9, Science 339: 823-826; Ran et al. (2015) In vivo genome editing using Staphylococcus aureus cas9, Nature 520: 186-191; Esvelt et al. (2013) Orthogonal cas9 proteins for ma-guided gene regulation and editing, Nature methods 10(11): 1116-1121, each of which is hereby incorporated by reference in its entirety for all purposes.

(iii) CRISPR/Cpf1

In some embodiments, the present disclosure provides methods of gene editing using a Type V CRISPR system. In some embodiments, the present disclosure provides methods of gene editing using CRISPR from Prevotella, Francisella, Acidaminococcus, Lachnospiraceae, and Moraxella (Cpf1).

The Cpf1 CRISPR systems of the present disclosure comprise i) a single endonuclease protein, and ii) a crRNA, wherein a portion of the 3′ end of crRNA contains the guide sequence complementary to a target nucleic acid. In this system, the Cpf1 nuclease is directly recruited to the target DNA by the crRNA. In some embodiments, guide sequences for Cpf1 must be at least 12 nt, 13 nt, 14 nt, 15 nt, or 16 nt in order to achieve detectable DNA cleavage, and a minimum of 14 nt, 15 nt, 16 nt, 17 nt, or 18 nt to achieve efficient DNA cleavage.

The Cpf1 systems of the present disclosure differ from Cas9 in a variety of ways. First, unlike Cas9, Cpf1 does not require a separate tracrRNA for cleavage. In some embodiments, Cpf1 crRNAs can be as short as about 42-44 bases long—of which 23-25 nt is guide sequence and 19 nt is the constitutive direct repeat sequence. In contrast, the combined Cas9 tracrRNA and crRNA synthetic sequences can be about 100 bases long.

Second, certain Cpf1 systems prefer a “TTN” PAM motif that is located 5′ upstream of its target. This is in contrast to the “NGG” PAM motifs located on the 3′ of the target DNA for common Cas9 systems such as Streptococcus pyogenes Cas9. In some embodiments, the uracil base immediately preceding the guide sequence cannot be substituted (Zetsche, B. et al. 2015. “Cpf1 Is a Single RNA-Guided Endonuclease of a Class 2 CRISPR-Cas System” Cell 163, 759-771, which is hereby incorporated by reference in its entirety for all purposes).

Third, the cut sites for Cpf1 are staggered by about 3-5 bases, which create “sticky ends” (Kim et al., 2016. “Genome-wide analysis reveals specificities of Cpf1 endonucleases in human cells” published online Jun. 6, 2016). These sticky ends with 3-5 nt overhangs are thought to facilitate NHEJ-mediated-ligation, and improve gene editing of DNA fragments with matching ends. The cut sites are in the 3′ end of the target DNA, distal to the 5′ end where the PAM is. The cut positions usually follow the 18th base on the non-hybridized strand and the corresponding 23rd base on the complementary strand hybridized to the crRNA.

Fourth, in Cpf1 complexes, the “seed” region is located within the first 5 nt of the guide sequence. Cpf1 crRNA seed regions are highly sensitive to mutations, and even single base substitutions in this region can drastically reduce cleavage activity (see Zetsche B. et al. 2015 “Cpf1 Is a Single RNA-Guided Endonuclease of a Class 2 CRISPR-Cas System” Cell 163, 759-771). Critically, unlike the Cas9 CRISPR target, the cleavage sites and the seed region of Cpf1 systems do not overlap. Additional guidance on designing Cpf1 crRNA targeting oligos is available on Zetsche B. et al. 2015. (“Cpf1 Is a Single RNA-Guided Endonuclease of a Class 2 CRISPR-Cas System” Cell 163, 759-771).

(iv) Guide RNA (gRNA)

In some embodiments, the guide RNA of the present disclosure comprises two coding regions, encoding for crRNA and tracrRNA, respectively. In other embodiments, the guide RNA is a single guide RNA (sgRNA) synthetic crRNA/tracrRNA hybrid. In other embodiments, the guide RNA is a crRNA for a Cpf1 endonuclease.

Persons having skill in the art will appreciate that, unless otherwise noted, all references to a single guide RNA (sgRNA) in the present disclosure can be read as referring to a guide RNA (gRNA). Therefore, embodiments described in the present disclosure which refer to a single guide RNA (sgRNA) will also be understood to refer to a guide RNA (gRNA).

The guide RNA is designed so as to recruit the CRISPR endonuclease to a target DNA region. In some embodiments, the present disclosure teaches methods of identifying viable target CRISPR landing sites, and designing guide RNAs for targeting the sites. For example, in some embodiments, the present disclosure teaches algorithms designed to facilitate the identification of CRISPR landing sites within target DNA regions.

In some embodiments, the present disclosure teaches use of software programs designed to identify candidate CRISPR target sequences on both strands of an input DNA sequence based on desired guide sequence length and a CRISPR motif sequence (PAM, protospacer adjacent motif) for a specified CRISPR enzyme. For example, target sites for Cpf1 from Francisella novicida U112, with PAM sequences TTN, may be identified by searching for 5′-TTN-3′ both on the input sequence and on the reverse-complement of the input. The target sites for Cpf1 from Lachnospiraceae bacterium and Acidaminococcus sp., with PAM sequences TTTN, may be identified by searching for 5′-TTTN-3′ both on the input sequence and on the reverse complement of the input. Likewise, target sites for Cas9 of S. thermophilus CRISPR, with PAM sequence NNAGAAW, may be identified by searching for 5′-Nx-NNAGAAW-3′ both on the input sequence and on the reverse-complement of the input. The PAM sequence for Cas9 of S. pyogenes is 5′-NGG-3′.

Since multiple occurrences in the genome of the DNA target site may lead to nonspecific genome editing, after identifying all potential sites, sequences may be filtered out based on the number of times they appear in the relevant reference genome or modular CRISPR construct. For those CRISPR enzymes for which sequence specificity is determined by a ‘seed’ sequence (such as the first 5 bp of the guide sequence for Cpf1-mediated cleavage) the filtering step may also account for any seed sequence limitations.

In some embodiments, algorithmic tools can also identify potential off target sites for a particular guide sequence. For example, in some embodiments Cas-Offinder can be used to identify potential off target sites for Cpf1 (see Kim et al., 2016. “Genome-wide analysis reveals specificities of Cpf1 endonucleases in human cells” Nature Biotechnology 34, 863-868). Any other publicly available CRISPR design/identification tool may also be used, including for example the Zhang lab crispr.mit.edu tool (see Hsu, et al. 2013 “DNA targeting specificity of RNA guided Cas9 nucleases” Nature Biotech 31, 827-832).

In some embodiments, the user may be allowed to choose the length of the seed sequence. The user may also be allowed to specify the number of occurrences of the seed: PAM sequence in a genome for purposes of passing the filter. The default is to screen for unique sequences. Filtration level is altered by changing both the length of the seed sequence and the number of occurrences of the sequence in the genome. The program may in addition or alternatively provide the sequence of a guide sequence complementary to the reported target sequence(s) by providing the reverse complement of the identified target sequence(s).

In the guide RNA, the “spacer/guide sequence” sequence is complementary to the protospacer sequence in the DNA target. The gRNA “scaffold” for a single stranded gRNA structure is recognized by the Cas protein.

In some embodiments, the transgenic plant, plant part, plant cell, or plant tissue culture taught in the present disclosure comprise a recombinant construct, which comprises at least one nucleic acid sequence encoding a guide RNA. In some embodiments, the nucleic acid is operably linked to a promoter. In some embodiments, the promoter is MtU6 promoter. In other embodiments, a recombinant construct further comprises a nucleic acid sequence encoding a Clustered regularly interspaced short palindromic repeats (CRISPR) endonuclease. In other embodiments, the guide RNA is capable of forming a complex with said CRISPR endonuclease, and said complex is capable of binding to and creating a double strand break in a genomic target sequence of said plant genome. In other embodiments, the CRISPR endonuclease is Cas9.

In some embodiments, the target sequence is a nucleic acid sequence encoding a seed storage protein. Most seed proteins are members of the cupin superfamily (e.g. legumins and vicilins), but in some dicotyledonous seeds, lectins are abundant, whereas in cereal grains, the prolamins and to a lesser extent the legumins are abundant. Non-limiting example of the seed storage protein is Glycinin (11S), beta-conglycinin (7S) proteins, Cupin protein, lectin protein, trypsin and protease inhibitor proteins. In some embodiments, the present disclosure teaches suppression and repression of the seed storage protein including Glycinin (11S), beta-conglycinin (7S) proteins, Cupin protein, lectin protein, trypsin and protease inhibitor proteins using genetic engineering techniques described herein.

Seed crops such as soybean plants are propagated for the stored protein, oil, and carbohydrates that specifically accumulate in seeds. Seeds accumulate protein as a source of carbon, nitrogen, and sulfur as well as triglycerides and carbohydrate reserves, which are used as a source of carbon and ultimately energy.

The present disclosure teaches the targeted gene-editing techniques for silencing, suppressing, or repressing expression of seed storage proteins including Glycinin (11S), beta-conglycinin (7S) proteins, Cupin protein, lectin protein, trypsin and protease inhibitor proteins. In some embodiments, the present disclosure teaches the targeted gene-editing techniques for silencing, suppressing, or repressing expression of soybean seed storage proteins, including, but not limited to β-conglycinin α, β-conglycinin α′, β-conglycinin β, Cysteine Protease, Glycinin 1, Glycinin 2, Glycinin 3, Glycinin 4, Glycinin 5, Glycinin 7, Kunitz-type Trypsin inhibitor, and Lectin. In other embodiments, the present disclosure teaches the targeted gene-editing techniques for silencing, suppressing, or repressing expression of β-conglycinin α, β-conglycinin α′, β-conglycinin J, Glycinin 1, Glycinin 4, Glycinin 5 and Kunitz-Trypsin inhibitor. In further embodiments, the present disclosure teaches the targeted gene-editing techniques for silencing, suppressing, or repressing expression of β-conglycinin α′, Glycinin 4 and Glycinin 5.

In some embodiments, seed storage proteins to be targeted for knock-out are not selected based on the size (e.g. molecular weight) of target protein but selected based on amino acid rebalancing profile taught in the present disclosure.

In some embodiments, the transgenic plant disclosed herein produces a seed with a rebalanced storage protein level when a gene encoding the storage protein is mutated by a gene-editing technique. In other embodiments, the level of proteins of interest in transgenic plants increases in seeds of the transgenic plants with the rebalanced storage protein level, when compared to seeds produced from a non-rebalanced transgenic plant. In further embodiments, the non-rebalanced transgenic plant has the recombinant DNA construct without mutation in the gene encoding the storage protein.

In some embodiments of the amino acid rebalancing based method, the disrupting of expression of the endogenous target gene is carried out by a gene-editing technology. In some embodiments, the gene-editing technology is a CRISPR/Cas system. In some embodiments, said CRISPR system comprises a nucleic acid molecule and an enzymatic protein, wherein the nucleic acid molecule is a guide RNA (gRNA) molecule, and the enzymatic protein is a Cas protein or Cas ortholog. In some embodiments, the disrupting of expression of the endogenous target gene does not utilize an RNAi-based technology. In some embodiments, the disruption is not due to the presence of an RNAi, an double-stranded RNA, an antisense or a sense fragment of the endogenous target gene. In some embodiments, at least two expression cassettes are stacked in tandem in the expression vector.

### Identification of Recombinant Plants

In some embodiments, recombinant plants of the disclosure are modified to express a trait to aid in their identification during processing, shipment, storage, and any upstream use ahead of processing for a food product. In some embodiments, the trait is phenotypic. In some embodiments, the trait is genetic. In some embodiments, the trait is not present in an end-product. That is, in some embodiments, the marker is designed so as to not be present in downstream processing of a grain, bean, fruit, or other plant part. In some embodiments, the trait is reduced or eliminated in an end-product. Various end-products are described herein and comprise at least a consumable or edible product.

When the trait is phenotypic, any portion of the plant can be modified to express the trait. In some embodiments, a plant is a soybean plant, and a phenotypic trait is expressed in a portion of the soybean selected from the group consisting of: testa, root, hilum, raphe, cotyledon, plumule, meristem, epicotyl, hypocotyl, microphyle, and combinations thereof. In some embodiments, the testa (e.g., seed coat) of the plant expresses a phenotypic trait. In some embodiments, the testa is modified to express a pigment. In some embodiments, the palisade layer of the testa is modified to express a pigment. In some embodiments, the parenchyma layer of a soybean expresses a pigment. In some embodiments, the parenchyma layer of a soybean does not expresses a pigment.

Phenotypic traits that can be expressed by recombinant plants of the disclosure can comprise a pigment or metabolite. In some embodiments, a pigment is selected from the group consisting of flavonoids (phlobaphenes, isoflavonoids, flavanones, flavones, flavonols, proanthocyanidins, anthocyanins), stilbenes, phenolic acids, monolignols, and combinations thereof. In some emboidments, a pigment comprises an anthocyanin. Anthocyanins are colored water-soluble pigments belonging to the phenolic group. The pigments are in glycosylated forms. Anthocyanins are responsible for the colors, red, purple, and blue. Therefore, in some embodiments, a portion of a plant (e.g., a soybean) comprises a red, purple, and/or blue pigment. In some embodiments, a portion of a soybean selected from the group consisting of testa, root, hilum, raphe, cotyledon, plumule, meristem, epicotyl, hypocotyl, microphyle, and combinations thereof expresses a red, purple, and/or blue pigment.

Any methods may be utilized to modify a soybean to express a pigment. Exemplary strategies are described herein at Example 23.

### Quantification of Recombinant Protein

Plants provide a platform for the production of recombinant proteins in terms of scalability, safety, and/or cost-effectiveness and thus serve as an alternative to conventional fermentation systems that use bacteria, yeast or mammalian cells. In some embodiments, conventional systems comprising bacteria, yeast, and/or mammalian cells may not be viable options to express a recombinant protein. In some embodiments, a system comprising bacteria may not be utilized to express a recombinant protein, such as any of the fusion proteins provided herein.

Accordingly, provided are methods comprising expressing a recombining protein in a plant. Also provided are methods of increasing yield of recombinant milk protein per acre in a soybean plant. Expressing recombinant milk proteins in plants has advantages such as ease of scaling up, low growth cost, optimized growth procedures, ease of producing complex proteins (e.g., fusion proteins), and/or low risk of contamination with human pathogens. As described herein, any plant can be utilized in the described methods. In some embodiments, the plant is a soybean plant.

In some embodiments, the yield of recombinant protein is determined at one or both of the transcriptional and post-transcriptional level.

In some embodiments, a method provided herein comprises determining weight of a recombinant protein expressed in a plant. The plant can be of any age, in some embodiments the plant is at least about 1, 2, 3, 4, 5, 6, 7, 8, 9, or up to about 10 months old. In some embodiments, the plant is at least about 1-2, 2-4, 3-5, 1-4, 2-6, or about 3-4 months old. Methods of determining weight of a recombinant protein can comprise grounding at least a portion of a plant. The portion of a plant can be at least a portion of a leaf, stem, seed, root, or combinations thereof. In some embodiments, a recombinant protein is selected from any of the proteins described herein. In some embodiments, the recombinant protein comprises a fusion protein. In some embodiments, the recombinant protein comprises a milk protein.

In some embodiments, a method provided herein comprises determining weight of recombinant milk protein. Any methods of determining weight of recombinant protein can be utilized. Suitable methods of determining weight of a recombinant protein can comprise: western blot, southern blot, northern blot, ELISA, and combinations thereof. In some embodiments, an assay utilizes a Malvern Mastersizer light scatter device.

In some embodiments, a method comprises determining expression of a recombinant protein. In an exemplary method, total RNA can be isolated by grinding young leaves or seeds collected from 3-4 month-old plants followed by northern blot analysis and RNA probe labelling and hybridization. In some embodiments, a method comprises grounding at least a portion of a plant suspected of expressing the recombinant protein (e.g., seed), contacting the ground portion with a solution comprising a lysis buffer, thereby forming a lysate. The lysate can then be centrifuged and/or run on a gel followed by contact with an antibody targeting a protein of interest. In some embodiments, the protein of interest comprises a milk protein.

In some embodiments, hulled beans are macerated/crushed/blended in an aqueous solution, and then filtered. Protein within the resulting filtrate is then separated via alcohol or acid precipitation followed by a second optional filtration. Recovered protein is then dried and weighed.

In some embodiments, at least about 2 lbs., 2.3 lbs., 2.6 lbs., 2.9 lbs., 3.2 lbs., 3.5 lbs., 3.8 lbs., 4.1 lbs., 4.4 lbs., 4.7 lbs., 5 lbs., 5.3 lbs., 5.6 lbs., 5.9 lbs., 6.2 lbs., 6.5 lbs., 6.8 lbs., 7.1 lbs., 7.4 lbs., 7.7 lbs., 8 lbs., or up to about 8.5 lbs. per acre of recombinant protein are generated. In some embodiments, at least about 2-3 lbs., 2-5 lbs., 4-6 lbs, 5-7 lbs., 5.5-6.5 lbs, or 5-6.5 lbs per acre of recombinant protein are generated. In some embodiments at least about 6.0 lbs. per acre of recombinant protein are generated. In some embodiments, up to about 1000 lbs./acre of recombinant protein are generated. In some embodiments, up to about 500 lbs./acre, 525 lbs./acre, 550 lbs./acre, 575 lbs./acre, 600 lbs./acre, 625 lbs./acre, 650 lbs./acre, 675 lbs./acre, 700 lbs./acre, 725 lbs./acre, 750 lbs./acre, 775 lbs./acre, 800 lbs./acre, 825 lbs./acre, 850 lbs./acre, 875 lbs./acre, 900 lbs./acre, 925 lbs./acre, 950 lbs./acre, 975 lbs./acre, or up to about 1000 lbs./acre of recombinant protein are generated.

In some embodiments, at least about 0.1 lbs./bushel, 0.2 lbs./bushel, 0.5 lbs./bushel 1 lbs./bushel 1.5 lbs./bushel 2 lbs./bushel 2.5 lbs./bushel 3 lbs./bushel 3.5 lbs./bushel 4 lbs./bushel 4.5 lbs./bushel 5 lbs./bushel 5.5 lbs./bushel 6 lbs./bushel 6.5 lbs./bushel 7 lbs./bushel 7.5 lbs./bushel 8 lbs./bushel 8.5 lbs./bushel 9 lbs./bushel 9.5 lbs./bushel 10 lbs./bushel 10.5 lbs./bushel 11 lbs./bushel 11.5 lbs./bushel 12 lbs./bushel 12.5 lbs./bushel 13 lbs./bushel 13.5 lbs./bushel 14 lbs./bushel 14.5 lbs./bushel 15 lbs./bushel of a recombinant protein are generated. In some embodiments, a plurality of transgenic soybean seed produce in the aggregate at least 0.21 pounds of recombinant milk protein per bushel.

In some embodiments, at least about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 30.5%, 31%, 31.5%, 32%, 32.5%, 33%, 33.5%, 34%, 34.5%, 35%, 35.5%, 36%, 36.5%, 37%, 37.5%, 38%, 38.5%, 39%, 39.5%, 40%, 40.5%, 41%, 41.5%, 42%, 42.5%, 43%, 43.5%, 44%, 44.5%, 45%, 45.5%, 46%, 46.5%, 47%, 47.5%, 48%, 48.5%, 49%, 49.5%, 50%, 50.5%, 51%, 51.5%, 52%, 52.5%, 53%, 53.5%, 54%, 54.5%, 55%, 55.5%, 56%, 56.5%, 57%, 57.5%, 58%, 58.5%, 59%, 59.5%, 60%, 60.5%, 61%, 61.5%, 62%, 62.5%, 63%, 63.5%, 64%, 64.5%, 65%, 65.5%, 66%, 66.5%, 67%, 67.5%, 68%, 68.5%, 69%, 69.5%, 70%, 70.5%, 71%, 71.5%, 72%, 72.5%, 73%, 73.5%, 74%, 74.5%, or up to about 75% of total protein weight of a plant seed mass of recombinant protein is generated.

In some embodiments, a transgenic seed produces at least about: 0.5 mg/seed, 1 mg/seed, 1.5 mg/seed, 2 mg/seed, 2.5 mg/seed, 3 mg/seed, 3.5 mg/seed, 4 mg/seed, 4.5 mg/seed, 5 mg/seed, 5.5 mg/seed, 6 mg/seed, 6.5 mg/seed, 7 mg/seed, 7.5 mg/seed, 8 mg/seed, 8.5 mg/seed, 9 mg/seed, 9.5 mg/seed, 10 mg/seed, 10.5 mg/seed, 11 mg/seed, 11.5 mg/seed, 12 mg/seed, 12.5 mg/seed, 13 mg/seed, 13.5 mg/seed, 14 mg/seed, 14.5 mg/seed, 15 mg/seed, 15.5 mg/seed, 16 mg/seed, 16.5 mg/seed, 17 mg/seed, 17.5 mg/seed, 18 mg/seed, 18.5 mg/seed, 19 mg/seed, 19.5 mg/seed, 20 mg/seed, 20.5 mg/seed, 21 mg/seed, 21.5 mg/seed, 22 mg/seed, 22.5 mg/seed, 23 mg/seed, 23.5 mg/seed, 24 mg/seed, 24.5 mg/seed, 25 mg/seed, 25.5 mg/seed, 26 mg/seed, 26.5 mg/seed, 27 mg/seed, 27.5 mg/seed, 28 mg/seed, 28.5 mg/seed, 29 mg/seed, 29.5 mg/seed, 30 mg/seed of recombinant protein. In some embodiments, the seed is of any plant described herein. In some embodiments, the seed is a soybean seed.

In some embodiments, a transgenic soybean seed in a mature fruit pod produces at least about 0.5 mg/seed, 1 mg/seed, 1.5 mg/seed, 2 mg/seed, 2.5 mg/seed, 3 mg/seed, 3.5 mg/seed, 4 mg/seed, 4.5 mg/seed, 5 mg/seed, 5.5 mg/seed, 6 mg/seed, 6.5 mg/seed, 7 mg/seed, 7.5 mg/seed, 8 mg/seed, 8.5 mg/seed, 9 mg/seed, 9.5 mg/seed, 10 mg/seed, 10.5 mg/seed, 11 mg/seed, 11.5 mg/seed, 12 mg/seed, 12.5 mg/seed, 13 mg/seed, 13.5 mg/seed, 14 mg/seed, 14.5 mg/seed, 15 mg/seed, 15.5 mg/seed, 16 mg/seed, 16.5 mg/seed, 17 mg/seed, 17.5 mg/seed, 18 mg/seed, 18.5 mg/seed, 19 mg/seed, 19.5 mg/seed, 20 mg/seed, 20.5 mg/seed, 21 mg/seed, 21.5 mg/seed, 22 mg/seed, 22.5 mg/seed, 23 mg/seed, 23.5 mg/seed, 24 mg/seed, 24.5 mg/seed, 25 mg/seed, 25.5 mg/seed, 26 mg/seed, 26.5 mg/seed, 27 mg/seed, 27.5 mg/seed, 28 mg/seed, 28.5 mg/seed, 29 mg/seed, 29.5 mg/seed, 30 mg/seed of recombinant protein. In some embodiments, the seed is of any plant described herein. In some embodiments, the seed is a soybean seed.

In some embodiments, a plurality of genetically modified plant (e.g., soybean) seeds produce in the aggregate at least about 2 lbs./acre, 27 lbs./acre, 52 lbs./acre, 77 lbs./acre, 102 lbs./acre, 127 lbs./acre, 152 lbs./acre, 177 lbs./acre, 202 lbs./acre, 227 lbs./acre, 252 lbs./acre, 277 lbs./acre, 302 lbs./acre, 327 lbs./acre, 352 lbs./acre, 377 lbs./acre, 402 lbs./acre, 427 lbs./acre, 452 lbs./acre, 477 lbs./acre, 502 lbs./acre, 527 lbs./acre, 552 lbs./acre, 577 lbs./acre, 602 lbs./acre, 627 lbs./acre, 652 lbs./acre, 677 lbs./acre, 702 lbs./acre, 727 lbs./acre, 752 lbs./acre, 777 lbs./acre, 802 lbs./acre, 827 lbs./acre, 852 lbs./acre, 877 lbs./acre, 902 lbs./acre, 927 lbs./acre, 952 lbs./acre, 977 lbs./acre, or up to about 1000 lbs./acre of recombinant protein.

### Agricultural Compositions

The recombinant plants described herein, and/or having characteristics as described herein, can be exposed to any of a number of agricultural formulations. These agricultural formulations can be added to the plant in the form of a liquid, a foam, or a dry product (e.g. to seeds of the plants, in furrow, foliar, etc.).

The agricultural compositions may be added to the recombinant plants to improve plant traits. In some examples, one or more compositions may be coated onto a seed. In some examples, one or more compositions may be coated onto a seedling. In some examples, one or more compositions may be coated onto a surface of a seed. In some examples, one or more compositions may be coated as a layer above a surface of a seed. In some examples, a composition that is coated onto a seed may be in liquid form, in dry product form, in foam form, in a form of a slurry of powder and water, or in a flowable seed treatment. In some examples, one or more compositions may be applied to a seed and/or seedling by spraying, immersing, coating, encapsulating, and/or dusting the seed and/or seedling with the one or more compositions.

Examples of agricultural compositions that may be added to the recombinant plants taught herein can include seed coatings for commercially important agricultural crops, for example, seed coatings for corn, soybean, canola, sorghum, potato, rice, vegetables, cereals, and oilseeds. In some examples, compositions may be sprayed on the plant aerial parts, or applied to the roots by inserting into furrows in which the plant seeds are planted, watering to the soil, or dipping the roots in a suspension of the composition. In some examples, compositions may be supplemented with trace metal ions, such as molybdenum ions, iron ions, manganese ions, or combinations of these ions. The agricultural compositions described herein can improve plant traits of the recombinant plants, such as promoting plant growth, maintaining high chlorophyll content in leaves, increasing fruit or seed numbers, and increasing fruit or seed unit weight (e.g. larger soybean yield).

Alternatively, the compositions may be inserted directly into the furrows into which the seed is planted or sprayed onto the plant leaves or applied by dipping the roots into a suspension of the composition. An effective amount of the composition can be used. In general, an effective amount is an amount sufficient to result in plants with improved traits (e.g. a desired soybean yield).

The formulation useful for these agricultural composition embodiments may include at least one member selected from the group consisting of: a tackifier, a microbial stabilizer, a fungicide, an antibacterial agent, a preservative, a stabilizer, a surfactant, an anti-complex agent, an herbicide, a nematicide, an insecticide, a plant growth regulator, a fertilizer, a rodenticide, a dessicant, a bactericide, a nutrient, and any combination thereof.

Any of the compositions described herein can include an agriculturally acceptable carrier. The carrier can be a solid carrier or liquid carrier, and in various forms including microspheres, powders, emulsions and the like. The carrier may be any one or more of a number of carriers that confer a variety of properties, such as increased stability, wettability, or dispersability. Wetting agents such as natural or synthetic surfactants, which can be nonionic or ionic surfactants, or a combination thereof can be included in the composition. Suitable formulations that may be prepared include wettable powders, granules, gels, agar strips or pellets, thickeners, and the like, microencapsulated particles, and the like, liquids such as aqueous flowables, aqueous suspensions, water-in-oil emulsions, etc.

In some embodiments, the agricultural carrier may be soil or a plant growth medium. Other agricultural carriers that may be used include water, fertilizers, plant-based oils, humectants, or combinations thereof. Alternatively, the agricultural carrier may be a solid, such as diatomaceous earth, loam, silica, alginate, clay, bentonite, zeolite, vermiculite, seed cases, other plant and animal products, or combinations, including granules, pellets, or suspensions. Mixtures of any of the aforementioned ingredients are also contemplated as carriers, such as but not limited to, pesta (flour and kaolin clay), agar or flour-based pellets in loam, sand, or clay, etc.

### Fertilizers

A fertilizer can be used to help promote the growth or provide nutrients to a seed, seedling, or plant. Non-limiting examples of fertilizers include nitrogen, phosphorous, potassium, calcium, sulfur, magnesium, boron, chloride, manganese, iron, zinc, copper, molybdenum, and selenium (or a salt thereof). Additional examples of fertilizers include one or more amino acids, salts, carbohydrates, vitamins, glucose, NaCl, yeast extract, NH₄H₂PO₄, (NH₄)₂SO₄, glycerol, valine, L-leucine, lactic acid, propionic acid, succinic acid, malic acid, citric acid, KH tartrate, xylose, lyxose, and lecithin. In one embodiment, the formulation can include a tackifier or adherent (referred to as an adhesive agent) to help bind other active agents to a substance (e.g., a surface of a seed). In one embodiment, adhesives are selected from the group consisting of: alginate, gums, starches, lecithins, formononetin, polyvinyl alcohol, alkali formononetinate, hesperetin, polyvinyl acetate, cephalins, Gum Arabic, Xanthan Gum, Mineral Oil, Polyethylene Glycol (PEG), Polyvinyl pyrrolidone (PVP), Arabino-galactan, Methyl Cellulose, PEG 400, Chitosan, Polyacrylamide, Polyacrylate, Polyacrylonitrile, Glycerol, Triethylene glycol, Vinyl Acetate, Gellan Gum, Polystyrene, Polyvinyl, Carboxymethyl cellulose, Gum Ghatti, and polyoxyethylene-polyoxybutylene block copolymers.

### Adhesives

In some embodiments, the agricultural compositions comprise an adhesive, and adhesives can be, e.g., a wax such as carnauba wax, beeswax, Chinese wax, shellac wax, spermaceti wax, candelilla wax, castor wax, ouricury wax, and rice bran wax, a polysaccharide (e.g., starch, dextrins, maltodextrins, alginate, and chitosans), a fat, oil, a protein (e.g., gelatin and zeins), gum arables, and shellacs. Adhesive agents can be non-naturally occurring compounds, e.g., polymers, copolymers, and waxes. For example, non-limiting examples of polymers that can be used as an adhesive agent include: polyvinyl acetates, polyvinyl acetate copolymers, ethylene vinyl acetate (EVA) copolymers, polyvinyl alcohols, polyvinyl alcohol copolymers, celluloses (e.g., ethylcelluloses, methylcelluloses, hydroxymethylcelluloses, hydroxypropylcelluloses, and carboxymethylcelluloses), polyvinylpyrolidones, vinyl chloride, vinylidene chloride copolymers, calcium lignosulfonates, acrylic copolymers, polyvinylacrylates, polyethylene oxide, acylamide polymers and copolymers, polyhydroxyethyl acrylate, methylacrylamide monomers, and polychloroprene.

### Surfactants

Non-limiting examples of surfactants include nitrogen-surfactant blends such as Prefer 28 (Cenex), Surf-N(US), Inhance (Brandt), P-28 (Wilfarm) and Patrol (Helena); esterified seed oils include Sun-It II (AmCy), MSO (UAP), Scoil (Agsco), Hasten (Wilfarm) and Mes-100 (Drexel); and organo-silicone surfactants include Silwet L77 (UAP), Silikin (Terra), Dyne-Amic (Helena), Kinetic (Helena), Sylgard 309 (Wilbur-Ellis) and Century (Precision). In one embodiment, the surfactant is present at a concentration of between 0.01% v/v to 10% v/v. In another embodiment, the surfactant is present at a concentration of between 0.1% v/v to 1% v/v.

### Fungicides

In some examples, a fungicide may include a compound or agent, whether chemical or biological, that can inhibit the growth of a fungus or kill a fungus. In some examples, a fungicide may include compounds that may be fungistatic or fungicidal. In some examples, fungicide can be a protectant, or agents that are effective predominantly on the seed surface, providing protection against seed surface-borne pathogens and providing some level of control of soil-borne pathogens. Non-limiting examples of protectant fungicides include captan, maneb, thiram, or fludioxonil.

In some examples, fungicide can be a systemic fungicide, which can be absorbed into the emerging seedling and inhibit or kill the fungus inside host plant tissues. Systemic fungicides used for seed treatment include, but are not limited to the following: azoxystrobin, carboxin, mefenoxam, metalaxyl, thiabendazole, trifloxystrobin, and various triazole fungicides, including difenoconazole, ipconazole, tebuconazole, and triticonazole. Mefenoxam and metalaxyl are primarily used to target the water mold fungi Pythium and Phytophthora. Some fungicides are preferred over others, depending on the plant species, either because of subtle differences in sensitivity of the pathogenic fungal species, or because of the differences in the fungicide distribution or sensitivity of the plants. In some examples, fungicide can be a biological control agent, such as a bacterium or fungus. Such organisms may be parasitic to the pathogenic fungi, or secrete toxins or other substances which can kill or otherwise prevent the growth of fungi. Any type of fungicide, particularly ones that are commonly used on plants, can be used.

### Control Agents

In some examples, the agricultural composition comprises a control agent, which has antibacterial properties. In one embodiment, the control agent with antibacterial properties is selected from the compounds described herein elsewhere. In another embodiment, the compound is Streptomycin, oxytetracycline, oxolinic acid, or gentamicin. Other examples of antibacterial compounds which can be used as part of a seed coating composition include those based on dichlorophene and benzylalcohol hemi formal (Proxel® from ICI or Acticide® RS from Thor Chemie and Kathon® MK 25 from Rohm & Haas) and isothiazolinone derivatives such as alkylisothiazolinones and benzisothiazolinones (Acticide® MBS from Thor Chemie).

### Growth Regulators

In some examples, a growth regulator is selected from the group consisting of: Abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N-6-benzyladenine, paclobutrazol, prohexadione phosphorotrithioate, 2,3,5-tri-iodobenzoic acid, trinexapac-ethyl and uniconazole. Additional non-limiting examples of growth regulators include brassinosteroids, cytokinines (e.g., kinetin and zeatin), auxins (e.g., indolylacetic acid and indolylacetyl aspartate), flavonoids and isoflavanoids (e.g., formononetin and diosmetin), phytoaixins (e.g., glyceolline), and phytoalexin-inducing oligosaccharides (e.g., pectin, chitin, chitosan, polygalacuronic acid, and oligogalacturonic acid), and gibellerins. Such agents are ideally compatible with the agricultural seed or seedling onto which the formulation is applied (e.g., it should not be deleterious to the growth or health of the plant). Furthermore, the agent is ideally one which does not cause safety concerns for human, animal or industrial use (e.g., no safety issues, or the compound is sufficiently labile that the commodity plant product derived from the plant contains negligible amounts of the compound).

### Nutrients

Some examples of nutrients can be selected from the group consisting of a nitrogen fertilizer including, but not limited to Urea, Ammonium nitrate, Ammonium sulfate, Non-pressure nitrogen solutions, Aqua ammonia, Anhydrous ammonia, Ammonium thiosulfate, Sulfur-coated urea, Urea-formaldehydes, IBDU, Polymer-coated urea, Calcium nitrate, Ureaform, and Methylene urea, phosphorous fertilizers such as Diammonium phosphate, Monoammonium phosphate, Ammonium polyphosphate, Concentrated superphosphate and Triple superphosphate, and potassium fertilizers such as Potassium chloride, Potassium sulfate, Potassium-magnesium sulfate, Potassium nitrate.

### Pests

Agricultural compositions of the disclosure, which may be added to the recombinant plants as taught herein, are sometimes combined with one or more pesticides. These additions provide the recombinant plants taught herein with a pest free or pest reduced growing environment.

The pesticides that are combined with the recombinant plants of the disclosure may target any of the pests mentioned below.

“Pest” includes but is not limited to, insects, fungi, bacteria, nematodes, mites, ticks and the like. Insect pests include insects selected from the orders Coleoptera, Diptera, Hymenoptera, Lepidoptera, Mallophaga, Homoptera, Hemiptera Orthroptera, Thysanoptera, Dermaptera, Isoptera, Anoplura, Siphonaptera, Trichoptera, etc., particularly Lepidoptera and Coleoptera.

Exemplary chemical compositions, which may be combined with the recombinant plants of the disclosure, include: Soybean Herbicides: Alachlor, Bentazone, Trifluralin, Chlorimuron-Ethyl, Cloransulam-Methyl, Fenoxaprop, Fomesafen, Flu-azifop, Glyphosate, Imazamox, Imazaquin, Imazethapyr, (S-)Metolachlor, Metribuzin, Pendimethalin, Tepraloxydim, Glufosinate; Soybean Insecticides: Lambda-cyhalothrin, Methomyl, Parathion, Thiocarb, Imidacloprid, Clothianidin, Thiamethoxam, Thiacloprid, Acetamiprid, Dinetofuran, Flubendiamide, Rynaxypyr, Cyazypyr, Spinosad, Spinotoram, Emamectin-Benzoate, Fipronil, Ethiprole, Deltamethrin, O-Cyfluthrin, gamma and lambda Cyhalothrin, 4-[[(6-Chlorpyridin-3-yl)methyl] (2,2-difluorethyl)amino]furan-2(5H)-on, Spirotetramat, Spinodiclofen, Triflumuron, Flonicamid, Thiodicarb, beta-Cyfluthrin; Soybean Fungicides: Azoxystrobin, Cyproconazole, Epoxiconazole, Flutriafol, Pyraclostrobin, Tebuconazole, Trifloxystrobin, Prothioconazole, Tetraconazole;

### Insecticidal Compositions

As aforementioned, agricultural compositions of the disclosure, which may be added to a recombinant plant as taught herein, are sometimes combined with (or contain) one or more insecticides.

In some embodiments, insecticidal compositions may be included in the compositions set forth herein, and can be applied to a plant or a part thereof simultaneously or in succession, with other compounds. Exemplary insecticides are provided in Table 18.

TABLE 18
Exemplary insecticides associated with various modes of action, which
can be combined with the recombinant plants of the disclosure

| | | | Physiological |
|---|---|---|---|
| Mode of Action | Compound class | Exemplary insecticides | function(s) affected |
| acetylcholinesterase | carbamates | Alanycarb, Aldicarb, | Nerve and |
| (AChE) inhibitors | | Bendiocarb, Benfuracarb, | muscle |
| | | Butocarboxim, | |
| | | Butoxycarboxim, Carbaryl, | |
| | | Carbofuran, Carbosulfan, | |
| | | Ethiofencarb, Fenobucarb, | |
| | | Formetanate, Furathiocarb, | |
| | | Isoprocarb, Methiocarb, | |
| | | Methomyl, Metolcarb, | |
| | | Oxamyl, Pirimicarb, Propoxur, | |
| | | Thiodicarb, Thiofanox, | |
| | | Triazamate, Trimethacarb, | |
| | | XMC, Xylylcarb | |
| acetylcholinesterase | organophosphates | Acephate, Azamethiphos, | Nerve and |
| (AChE) inhibitors | | Azinphos-ethyl, Azinphos- | muscle |
| | | methyl, Cadusafos, | |
| | | Chlorethoxyfos, | |
| | | Chlorfenvinphos, | |
| | | Chlormephos, Chlorpyrifos, | |
| | | Chlorpyrifos-methyl, | |
| | | Coumaphos, Cyanophos, | |
| | | Demeton-S-methyl, Diazinon, | |
| | | Dichlorvos/DDVP, | |
| | | Dicrotophos, Dimethoate, | |
| | | Dimethylvinphos, Disulfoton, | |
| | | EPN, Ethion, Ethoprophos, | |
| | | Famphur, Fenamiphos, | |
| | | Fenitrothion, Fenthion, | |
| | | Fosthiazate, Heptenophos, | |
| | | Imicyafos, Isofenphos, | |
| | | Isopropyl O- | |
| | | (methoxyaminothio- | |
| | | phosphoryl) salicylate, | |
| | | Isoxathion, Malathion, | |
| | | Mecarbam, Methamidophos, | |
| | | Methidathion, Mevinphos, | |
| | | Monocrotophos, Naled, | |
| | | Omethoate, Oxydemeton- | |
| | | methyl, Parathion, Parathion- | |
| | | methyl, Phenthoate, Phorate, | |
| | | Phosalone, Phosmet, | |
| | | Phosphamidon, Phoxim, | |
| | | Pirimiphos-methyl, | |
| | | Profenofos, Propetamphos, | |
| | | Prothiofos, Pyraclofos, | |
| | | Pyridaphenthion, Quinalphos, | |
| | | Sulfotep, Tebupirimfos, | |
| | | Temephos, Terbufos, | |
| | | Tetrachlorvinphos, Thiometon, | |
| | | Triazophos, Trichlorfon, | |
| | | Vamidothion | |
| GABA-gated | cyclodiene | Chlordane, Endosulfan | Nerve and |
| chloride channel | organochlorines | | muscle |
| blockers | | | |
| GABA-gated | phenylpyrazoles | Ethiprole, Fipronil | Nerve and |
| chloride channel | (Fiproles) | | muscle |
| blockers | | | |
| sodium channel | pyrethroids, | Acrinathrin, Allethrin, | Nerve and |
| modulators | pyrethrins | Bifenthrin, Bioallethrin, | muscle |
| | | Bioallethrin S-cyclopentenyl, | |
| | | Bioresmethrin, Cycloprothrin, | |
| | | Cyfluthrin, Cyhalothrin, | |
| | | Cypermethrin, Cyphenothrin | |
| | | [(1R)-trans-isomers], | |
| | | Deltamethrin, Empenthrin | |
| | | [(EZ)-(1R)-isomers], | |
| | | Esfenvalerate, Etofenprox, | |
| | | Fenpropathrin, Fenvalerate, | |
| | | Flucythrinate, Flumethrin, | |
| | | Halfenprox, Kadathrin, | |
| | | Phenothrin [(1R)-trans- | |
| | | isomer], Prallethrin, Pyrethrins | |
| | | (pyrethrum), Resmethrin, | |
| | | Silafluofen, Tefluthrin, | |
| | | Tetramethrin, Tetramethrin | |
| | | [(1R)-isomers], Tralomethrin, | |
| | | Transfluthrin, alpha- | |
| | | Cypermethrin, beta-Cyfluthrin, | |
| | | beta-Cypermethrin, d-cis-trans | |
| | | Allethrin, d-trans Allethrin, | |
| | | gamma-Cyhalothrin, lambda- | |
| | | Cyhalothrin, tau-Fluvalinate, | |
| | | theta-Cypermethrin, zeta- | |
| | | Cypermethrin | |
| sodium channel | DDT, | DDT, | Nerve and |
| modulators | methoxychlor | methoxychlor | muscle |
| nicotinic | neonicotinoids | Acetamiprid, Clothianidin, | Nerve and |
| acetylcholine | | Dinotefuran, Imidacloprid, | muscle |
| receptor (nAChR) | | Nitenpyram, Thiacloprid, | |
| competitive | | Thiamethoxam | |
| modulators | | | |
| nicotinic | nicotine | nicotine | Nerve and |
| acetylcholine | | | muscle |
| receptor (nAChR) | | | |
| competitive | | | |
| modulators | | | |
| nicotinic | sulfoximines | sulfoxaflor | Nerve and |
| acetylcholine | | | muscle |
| receptor (nAChR) | | | |
| competitive | | | |
| modulators | | | |
| nicotinic | butenolides | Flupyradifurone | Nerve and |
| acetylcholine | | | muscle |
| receptor (nAChR) | | | |
| competitive | | | |
| modulators | | | |
| nicotinic | spinosyns | Spinetoram, Spinosad | Nerve and |
| acetylcholine | | | muscle |
| receptor (nAChR) | | | |
| allosteric | | | |
| modulators | | | |
| Glutamate-gated | avermectins, | Abamectin, Emamectin | Nerve and |
| chloride channel | milbemycins | benzoate, Lepimectin, | muscle |
| (GluCl) allosteric | | Milbemectin | |
| modulators | | | |
| juvenile hormone | juvenile hormone | Hydroprene, Kinoprene, | Growth |
| mimics | analogues | Methoprene | |
| juvenile hormone | Fenoxycarb | Fenoxycarb | Growth |
| mimics | | | |
| juvenile hormone | Pyriproxyfen | Pyriproxyfen | Growth |
| mimics | | | |
| miscellaneous non- | alkyl halides | Methyl bromide and other | Unknown or |
| specific (multi-site) | | alkyl halides | non-specific |
| inhibitors | | | |
| miscellaneous non- | Chloropicrin | Chloropicrin | Unknown or |
| specific (multi-site) | | | non-specific |
| inhibitors | | | |
| miscellaneous non- | fluorides | Cryolite, sulfuryl fluoride | Unknown or |
| specific (multi-site) | | | non-specific |
| inhibitors | | | |
| miscellaneous non- | borates | Borax, Boric acid, Disodium | Unknown or |
| specific (multi-site) | | octaborate, Sodium borate, | non-specific |
| inhibitors | | Sodium metaborate | |
| miscellaneous non- | tartar emetic | tartar emetic | Unknown or |
| specific (multi-site) | | | non-specific |
| inhibitors | | | |
| miscellaneous non- | methyl | Dazomet, Metam | Unknown or |
| specific (multi-site) | isothiocyanate | | non-specific |
| inhibitors | generators | | |
| modulators of | Pyridine | Pymetrozine, Pyrifluquinazon | Nerve and |
| chordotonal organs | azomethine | | muscle |
| | derivatives | | |
| mite growth | Clofentezine, | Clofentezine, | Growth |
| inhibitors | Diflovidazin, | Diflovidazin, | |
| | Hexythiazox | Hexythiazox | |
| mite growth | Etoxazole | Etoxazole | Growth |
| inhibitors | | | |
| microbial | Bacillus | Bt var. aizawai, Bt var. | Midgut |
| disruptors of insect | thuringiensis and | israelensis, Bt var. kurstaki, Bt | |
| midgut membranes | the insecticidal | var. tenebrionensis | |
| | proteins they | | |
| | produce | | |
| microbial | Bacillus | Bacillus | Midgut |
| disruptors of insect | sphaericus | sphaericus | |
| midgut membranes | | | |
| inhibitors of | Diafenthiuron | Diafenthiuron | Respiration |
| mitochondrial ATP | | | |
| synthase | | | |
| inhibitors of | organotin | Azocyclotin, Cyhexatin, | Respiration |
| mitochondrial ATP | miticides | Fenbutatin oxide | |
| synthase | | | |
| inhibitors of | Propargite | Propargite | Respiration |
| mitochondrial ATP | | | |
| synthase | | | |
| inhibitors of | Tetradifon | Tetradifon | Respiration |
| mitochondrial ATP | | | |
| synthase | | | |
| uncouplers of | Chlorfenapyr, | Chlorfenapyr, | Respiration |
| oxidative | DNOC, | DNOC, | |
| phosphorylation via | Sulfuramid | Sulfuramid | |
| disruption of the | | | |
| proton gradient | | | |
| Nicotinic | nereistoxin | Bensultap, Cartap | Nerve and |
| acetylcholine | analogues | hydrochloride, Thiocyclam, | muscle |
| receptor (nAChR) | | Thiosultap-sodium | |
| channel blockers | | | |
| inhibitors of chitin | benzoylureas | Bistrifluron, Chlorfluazuron, | Growth |
| biosynthesis, type 0 | | Diflubenzuron, Flucycloxuron, | |
| | | Flufenoxuron, Hexaflumuron, | |
| | | Lufenuron, Novaluron, | |
| | | Noviflumuron, Teflubenzuron, | |
| | | Triflumuron | |
| inhibitors of chitin | Buprofezin | Buprofezin | Growth |
| biosynthesis, type 1 | | | |
| moulting disruptor, | Cyromazine | Cyromazine | Growth |
| Dipteran | | | |
| ecdysone receptor | diacylhydrazines | Chromafenozide, | Growth |
| agonists | | Halofenozide, | |
| | | Methoxyfenozide, | |
| | | Tebufenozide | |
| octopamine | Amitraz | Amitraz | Nerve and |
| receptor agonists | | | muscle |
| mitochondrial | Hydramethylnon | Hydramethylnon | Respiration |
| complex III | | | |
| electron transport | | | |
| inhibitors | | | |
| mitochondrial | Acequinocyl | Acequinocyl | Respiration |
| complex III | | | |
| electron transport | | | |
| inhibitors | | | |
| mitochondrial | Fluacrypyrim | Fluacrypyrim | Respiration |
| complex III | | | |
| electron transport | | | |
| inhibitors | | | |
| mitochondrial | Bifenazate | Bifenazate | Respiration |
| complex III | | | |
| electron transport | | | |
| inhibitors | | | |
| mitochondrial | Meti acaricides | Fenazaquin, Fenpyroximate, | Respiration |
| complex I electron | and insecticides | Pyridaben, Pyrimidifen, | |
| transport inhibitors | | Tebufenpyrad, Tolfenpyrad | |
| mitochondrial | Rotenone | Rotenone | Respiration |
| complex I electron | | | |
| transport inhibitors | | | |
| voltage-dependent | oxadiazines | Indoxacarb | Nerve and |
| sodium channel | | | muscle |
| blockers | | | |
| voltage-dependent | semicarbazones | Metaflumizone | Nerve and |
| sodium channel | | | muscle |
| blockers | | | |
| inhibitors of acetyl | tetronic and | Spirodiclofen, Spiromesifen, | Growth |
| CoA carboxylase | tetramic acid | Spirotetramat | |
| | derivatives | | |
| mitochondrial | phosphides | Aluminium phosphide, | Respiration |
| complex IV | | Calcium phosphide, | |
| electron transport | | Phosphine, Zinc phosphide | |
| inhibitors | | | |
| mitochondrial | cyanides | Calcium cyanide, Potassium | Respiration |
| complex IV | | cyanide, Sodium cyanide | |
| electron transport | | | |
| inhibitors | | | |
| mitochondrial | beta-ketonitrile | Cyenopyrafen, Cyflumetofen | Respiration |
| complex II electron | derivatives | | |
| transport inhibitors | | | |
| mitochondrial | carboxanilides | Pyflubumide | Respiration |
| complex II electron | | | |
| transport inhibitors | | | |
| ryanodine receptor | diamides | Chlorantraniliprole, | Nerve and |
| modulators | | Cyantraniliprole, | muscle |
| | | Flubendiamide | |
| Chordotonal organ | Flonicamid | Flonicamid | Nerve and |
| modulators - | | | muscle |
| undefined target | | | |
| site | | | |
| compounds of | Azadirachtin | Azadirachtin | Unknown |
| unknown or | | | |
| uncertain mode of | | | |
| action | | | |
| compounds of | Benzoximate | Benzoximate | Unknown |
| unknown or | | | |
| uncertain mode of | | | |
| action | | | |
| compounds of | Bromopropylate | Bromopropylate | Unknown |
| unknown or | | | |
| uncertain mode of | | | |
| action | | | |
| compounds of | Chinomethionat | Chinomethionat | Unknown |
| unknown or | | | |
| uncertain mode of | | | |
| action | | | |
| compounds of | Dicofol | Dicofol | Unknown |
| unknown or | | | |
| uncertain mode of | | | |
| action | | | |
| compounds of | lime sulfur | lime sulfur | Unknown |
| unknown or | | | |
| uncertain mode of | | | |
| action | | | |
| compounds of | Pyridalyl | Pyridalyl | Unknown |
| unknown or | | | |
| uncertain mode of | | | |
| action | | | |
| compounds of | sulfur | sulfur | Unknown |
| unknown or | | | |
| uncertain mode of | | | |
| action | | | |

TABLE 19
Exemplary list of pesticides, which can be combined
with the recombinant plants of the disclosure

| Category | Compounds |
|---|---|
| INSECTICIDES | |
| arsenical insecticides | calcium arsenate |
| | copper acetoarsenite |
| | copper arsenate |
| | lead arsenate |
| | potassium arsenite |
| | sodium arsenite |
| botanical insecticides | allicin |
| | anabasine |
| | azadirachtin |
| | carvacrol |
| | d-limonene |
| | matrine |
| | nicotine |
| | nornicotine |
| | oxymatrine |
| | pyrethrins |
| | cinerins |
| | cinerin I |
| | cinerin II |
| | jasmolin I |
| | jasmolin II |
| | pyrethrin I |
| | pyrethrin II |
| | quassia |
| | rhodojaponin-III |
| | rotenone |
| | ryania |
| | sabadilla |
| | sanguinarine |
| | triptolide |
| carbamate insecticides | bendiocarb |
| | carbaryl |
| benzofuranyl methylcarbamate | benfuracarb |
| insecticides | carbofuran |
| | carbosulfan |
| | decarbofuran |
| | furathiocarb |
| dimethylcarbamate insecticides | dimetan |
| | dimetilan |
| | hyquincarb |
| | isolan |
| | pirimicarb |
| | pyramat |
| | pyrolan |
| oxime carbamate insecticides | alanycarb |
| | aldicarb |
| | aldoxycarb |
| | butocarboxim |
| | butoxycarboxim |
| | methomyl |
| | nitrilacarb |
| | oxamyl |
| | tazimcarb |
| | thiocarboxime |
| | thiodicarb |
| | thiofanox |
| phenyl methylcarbamate insecticides | allyxycarb |
| | aminocarb |
| | bufencarb |
| | butacarb |
| | carbanolate |
| | cloethocarb |
| | CPMC |
| | dicresyl |
| | dimethacarb |
| | dioxacarb |
| | EMPC |
| | ethiofencarb |
| | fenethacarb |
| | fenobucarb |
| | isoprocarb |
| | methiocarb |
| | metolcarb |
| | mexacarbate |
| | promacyl |
| | promecarb |
| | propoxur |
| | trimethacarb |
| | XMC |
| | xylylcarb |
| diamide insecticides | broflanilide |
| | chlorantraniliprole |
| | cyantraniliprole |
| | cyclaniliprole |
| | cyhalodiamide |
| | flubendiamide |
| | tetraniliprole |
| dinitrophenol insecticides | dinex |
| | dinoprop |
| | dinosam |
| | DNOC |
| fluorine insecticides | barium hexafluorosilicate |
| | cryolite |
| | flursulamid |
| | sodium fluoride |
| | sodium hexafluorosilicate |
| | sulfluramid |
| formamidine insecticides | amitraz |
| | chlordimeform |
| | formetanate |
| | formparanate |
| | medimeform |
| | semiamitraz |
| fumigant insecticides | acrylonitrile |
| | carbon disulfide |
| | carbon tetrachloride |
| | carbonyl sulfide |
| | chloroform |
| | chloropicrin |
| | cyanogen |
| | para-dichlorobenzene |
| | 1,2-dichloropropane |
| | dithioether |
| | ethyl formate |
| | ethylene dibromide |
| | ethylene dichloride |
| | ethylene oxide |
| | hydrogen cyanide |
| | methyl bromide |
| | methyl iodide |
| | methylchloroform |
| | methylene chloride |
| | naphthalene |
| | phosphine |
| | sodium tetrathiocarbonate |
| | sulfuryl fluoride |
| | tetrachloroethane |
| inorganic insecticides | borax |
| | boric acid |
| | calcium polysulfide |
| | copper oleate |
| | diatomaceous earth |
| | mercurous chloride |
| | potassium thiocyanate |
| | silica gel |
| | sodium thiocyanate |
| insect growth regulators | |
| chitin synthesis inhibitors | buprofezin |
| | cyromazine |
| benzoylphenylurea chitin synthesis | bistrifluron |
| inhibitors | chlorbenzuron |
| | chlorfluazuron |
| | dichlorbenzuron |
| | diflubenzuron |
| | flucycloxuron |
| | flufenoxuron |
| | hexaflumuron |
| | lufenuron |
| | novaluron |
| | noviflumuron |
| | penfluron |
| | teflubenzuron |
| | triflumuron |
| juvenile hormone mimics | dayoutong |
| | epofenonane |
| | fenoxycarb |
| | hydroprene |
| | kinoprene |
| | methoprene |
| | pyriproxyfen |
| | triprene |
| juvenile hormones | juvenile hormone I |
| | juvenile hormone II |
| | juvenile hormone III |
| moulting hormone agonists | chromafenozide |
| | furan tebufenozide |
| | halofenozide |
| | methoxyfenozide |
| | tebufenozide |
| | yishijing |
| moulting hormones | α-ecdysone |
| | ecdysterone |
| moulting inhibitors | diofenolan |
| precocenes | precocene I |
| | precocene II |
| | precocene III |
| unclassified insect growth regulators | dicyclanil |
| macrocyclic lactone insecticides | |
| avermectin insecticides | abamectin |
| | doramectin |
| | emamectin |
| | eprinomectin |
| | ivermectin |
| | selamectin |
| milbemycin insecticides | lepimectin |
| | milbemectin |
| | milbemycin oxime |
| | moxidectin |
| spinosyn insecticides | spinetoram |
| | spinosad |
| neonicotinoid insecticides | |
| nitroguanidine neonicotinoid insecticides | clothianidin |
| | dinotefuran |
| | imidacloprid |
| | imidaclothiz |
| | thiamethoxam |
| nitromethylene neonicotinoid insecticides | nitenpyram |
| | nithiazine |
| pyridylmethylamine neonicotinoid | acetamiprid |
| insecticides | imidacloprid |
| | nitenpyram |
| | paichongding |
| | thiacloprid |
| nereistoxin analogue insecticides | bensultap |
| | cartap |
| | polythialan |
| | thiocyclam |
| | thiosultap |
| organochlorine insecticides | bromo-DDT |
| | camphechlor |
| | DDT |
| | pp′-DDT |
| | ethyl-DDD |
| | HCH |
| | gamma-HCH |
| | lindane |
| | methoxychlor |
| | pentachlorophenol |
| | TDE |
| cyclodiene insecticides | aldrin |
| | bromocyclen |
| | chlorbicyclen |
| | chlordane |
| | chlordecone |
| | dieldrin |
| | dilor |
| | endosulfan |
| | alpha-endosulfan |
| | endrin |
| | HEOD |
| | heptachlor |
| | HHDN |
| | isobenzan |
| | isodrin |
| | kelevan |
| | mirex |
| organophosphorus insecticides | |
| organophosphate insecticides | bromfenvinfos |
| | calvinphos |
| | chlorfenvinphos |
| | crotoxyphos |
| | dichlorvos |
| | dicrotophos |
| | dimethylvinphos |
| | fospirate |
| | heptenophos |
| | methocrotophos |
| | mevinphos |
| | monocrotophos |
| | naled |
| | naftalofos |
| | phosphamidon |
| | propaphos |
| | TEPP |
| | tetrachlorvinphos |
| organothiophosphate insecticides | dioxabenzofos |
| | fosmethilan |
| | phenthoate |
| aliphatic organothiophosphate | acethion |
| insecticides | acetophos |
| | amiton |
| | cadusafos |
| | chlorethoxyfos |
| | chlormephos |
| | demephion |
| | demephion-O |
| | demephion-S |
| | demeton |
| | demeton-O |
| | demeton-S |
| | demeton-methyl |
| | demeton-O-methyl |
| | demeton-S-methyl |
| | demeton-S-methylsulphon |
| | disulfoton |
| | ethion |
| | ethoprophos |
| | IPSP |
| | isothioate |
| | malathion |
| | methacrifos |
| | methylacetophos |
| | oxydemeton-methyl |
| | oxydeprofos |
| | oxydisulfoton |
| | phorate |
| | sulfotep |
| | terbufos |
| | thiometon |
| aliphatic amide organothiophosphate | amidithion |
| insecticides | cyanthoate |
| | dimethoate |
| | ethoate-methyl |
| | formothion |
| | mecarbam |
| | omethoate |
| | prothoate |
| | sophamide |
| | vamidothion |
| oxime organothiophosphate insecticides | chlorphoxim |
| | phoxim |
| | phoxim-methyl |
| heterocyclic organothiophosphate | azamethiphos |
| insecticides | colophonate |
| | coumaphos |
| | coumithoate |
| | dioxathion |
| | endothion |
| | menazon |
| | morphothion |
| | phosalone |
| | pyraclofos |
| | pyrazothion |
| | pyridaphenthion |
| | quinothion |
| benzothiopyran organothiophosphate | dithicrofos |
| insecticides | thicrofos |
| benzotriazine organothiophosphate | azinphos-ethyl |
| insecticides | azinphos-methyl |
| isoindole organothiophosphate | dialifos |
| insecticides | phosmet |
| isoxazole organothiophosphate | isoxathion |
| insecticides | zolaprofos |
| pyrazolopyrimidine organothiophosphate | chlorprazophos |
| insecticides | pyrazophos |
| pyridine organothiophosphate | chlorpyrifos |
| insecticides | chlorpyrifos-methyl |
| pyrimidine organothiophosphate | butathiofos |
| insecticides | diazinon |
| | etrimfos |
| | lirimfos |
| | pirimioxyphos |
| | pirimiphos-ethyl |
| | pirimiphos-methyl |
| | primidophos |
| | pyrimitate |
| | tebupirimfos |
| quinoxaline organothiophosphate | quinalphos |
| insecticides | quinalphos-methyl |
| thiadiazole organothiophosphate | athidathion |
| insecticides | lythidathion |
| | methidathion |
| | prothidathion |
| triazole organothiophosphate insecticides | isazofos |
| | triazophos |
| phenyl organothiophosphate insecticides | azothoate |
| | bromophos |
| | bromophos-ethyl |
| | carbophenothion |
| | chlorthiophos |
| | cyanophos |
| | cythioate |
| | dicapthon |
| | dichlofenthion |
| | etaphos |
| | famphur |
| | fenchlorphos |
| | fenitrothion |
| | fensulfothion |
| | fenthion |
| | fenthion-ethyl |
| | heterophos |
| | jodfenphos |
| | mesulfenfos |
| | parathion |
| | parathion-methyl |
| | phenkapton |
| | phosnichlor |
| | profenofos |
| | prothiofos |
| | sulprofos |
| | temephos |
| | trichlormetaphos-3 |
| | trifenofos |
| | xiaochongliulin |
| phosphonate insecticides | butonate |
| | trichlorfon |
| phosphonothioate insecticides | mecarphon |
| phenyl ethylphosphonothioate | fonofos |
| insecticides | trichloronat |
| phenyl phenylphosphonothioate | cyanofenphos |
| insecticides | EPN |
| | leptophos |
| phosphoramidate insecticides | crufomate |
| | fenamiphos |
| | fosthietan |
| | mephosfolan |
| | phosfolan |
| | phosfolan-methyl |
| | pirimetaphos |
| phosphoramidothioate insecticides | acephate |
| | chloramine phosphorus |
| | isocarbophos |
| | isofenphos |
| | isofenphos-methyl |
| | methamidophos |
| | phosglycin |
| | propetamphos |
| phosphorodiamide insecticides | dimefox |
| | mazidox |
| | mipafox |
| | schradan |
| oxadiazine insecticides | indoxacarb |
| oxadiazolone insecticides | metoxadiazone |
| phthalimide insecticides | dialifos |
| | phosmet |
| | tetramethrin |
| physical insecticides | maltodextrin |
| desiccant insecticides | boric acid |
| | diatomaceous earth |
| | silica gel |
| pyrazole insecticides | chlorantraniliprole |
| | cyantraniliprole |
| | cyclaniliprole |
| | dimetilan |
| | isolan |
| | tebufenpyrad |
| | tetraniliprole |
| | tolfenpyrad |
| phenylpyrazole insecticides | acetoprole |
| | ethiprole |
| | fipronil |
| | flufiprole |
| | pyraclofos |
| | pyrafluprole |
| | pyriprole |
| | pyrolan |
| | vaniliprole |
| pyrethroid insecticides | |
| pyrethroid ester insecticides | acrinathrin |
| | allethrin |
| | bioallethrin |
| | esdépalléthrine |
| | barthrin |
| | bifenthrin |
| | kappa-bifenthrin |
| | bioethanomethrin |
| | brofenvalerate |
| | brofluthrinate |
| | bromethrin |
| | butethrin |
| | chlorempenthrin |
| | cyclethrin |
| | cycloprothrin |
| | cyfluthrin |
| | beta-cyfluthrin |
| | cyhalothrin |
| | gamma-cyhalothrin |
| | lambda-cyhalothrin |
| | cypermethrin |
| | alpha-cypermethrin |
| | beta-cypermethrin |
| | theta-cypermethrin |
| | zeta-cypermethrin |
| | cyphenothrin |
| | deltamethrin |
| | dimefluthrin |
| | dimethrin |
| | empenthrin |
| | d-fanshiluquebingjuzhi |
| | chloroprallethrin |
| | fenfluthrin |
| | fenpirithrin |
| | fenpropathrin |
| | fenvalerate |
| | esfenvalerate |
| | flucythrinate |
| | fluvalinate |
| | tau-fluvalinate |
| | furamethrin |
| | furethrin |
| | heptafluthrin |
| | imiprothrin |
| | japothrins |
| | kadethrin |
| | methothrin |
| | metofluthrin |
| | epsilon-metofluthrin |
| | momfluorothrin |
| | epsilon-momfluorothrin |
| | pentmethrin |
| | permethrin |
| | biopermethrin |
| | transpermethrin |
| | phenothrin |
| | prallethrin |
| | profluthrin |
| | proparthrin |
| | pyresmethrin |
| | renofluthrin |
| | meperfluthrin |
| | resmethrin |
| | bioresmethrin |
| | cismethrin |
| | tefluthrin |
| | kappa-tefluthrin |
| | terallethrin |
| | tetramethrin |
| | tetramethylfluthrin |
| | tralocythrin |
| | tralomethrin |
| | transfluthrin |
| | valerate |
| pyrethroid ether insecticides | etofenprox |
| | flufenprox |
| | halfenprox |
| | protrifenbute |
| | silafluofen |
| pyrethroid oxime insecticides | sulfoxime |
| | thiofluoximate |
| pyrimidinamine insecticides | flufenerim |
| | pyrimidifen |
| pyrrole insecticides | chlorfenapyr |
| quaternary ammonium insecticides | sanguinarine |
| sulfoximine insecticides | sulfoxaflor |
| tetramic acid insecticides | spirotetramat |
| tetronic acid insecticides | spiromesifen |
| thiazole insecticides | clothianidin |
| | imidaclothiz |
| | thiamethoxam |
| | thiapronil |
| thiazolidine insecticides | tazimcarb |
| | thiacloprid |
| thiourea insecticides | diafenthiuron |
| urea insecticides | flucofuron |
| | sulcofuron |
| zwitterionic insecticides | dicloromezotiaz |
| | triflumezopyrim |
| unclassified insecticides | afidopyropen |
| | afoxolaner |
| | allosamidin |
| | closantel |
| | copper naphthenate |
| | crotamiton |
| | EXD |
| | fenazaflor |
| | fenoxacrim |
| | flometoquin |
| | flonicamid |
| | fluhexafon |
| | flupyradifurone |
| | fluralaner |
| | fluxametamide |
| | hydramethylnon |
| | isoprothiolane |
| | jiahuangchongzong |
| | malonoben |
| | metaflumizone |
| | nifluridide |
| | plifenate |
| | pyridaben |
| | pyridalyl |
| | pyrifluquinazon |
| | rafoxanide |
| | thuringiensin |
| | triarathene |
| | triazamate |
| ACARICIDES | |
| botanical acaricides | carvacrol |
| | sanguinarine |
| bridged diphenyl acaricides | azobenzene |
| | benzoximate |
| | benzyl benzoate |
| | bromopropylate |
| | chlorbenside |
| | chlorfenethol |
| | chlorfenson |
| | chlorfensulphide |
| | chlorobenzilate |
| | chloropropylate |
| | cyflumetofen |
| | DDT |
| | dicofol |
| | diphenyl sulfone |
| | dofenapyn |
| | fenson |
| | fentrifanil |
| | fluorbenside |
| | genit |
| | hexachlorophene |
| | phenproxide |
| | proclonol |
| | tetradifon |
| | tetrasul |
| carbamate acaricides | benomyl |
| | carbanolate |
| | carbaryl |
| | carbofuran |
| | methiocarb |
| | metolcarb |
| | promacyl |
| | propoxur |
| oxime carbamate acaricides | aldicarb |
| | butocarboxim |
| | oxamyl |
| | thiocarboxime |
| | thiofanox |
| carbazate acaricides | bifenazate |
| dinitrophenol acaricides | binapacryl |
| | dinex |
| | dinobuton |
| | dinocap |
| | dinocap-4 |
| | dinocap-6 |
| | dinocton |
| | dinopenton |
| | dinosulfon |
| | dinoterbon |
| | DNOC |
| formamidine acaricides | amitraz |
| | chlordimeform |
| | chloromebuform |
| | formetanate |
| | formparanate |
| | medimeform |
| | semiamitraz |
| macrocyclic lactone acaricides | tetranactin |
| avermectin acaricides | abamectin |
| | doramectin |
| | eprinomectin |
| | ivermectin |
| | selamectin |
| milbemycin acaricides | milbemectin |
| | milbemycin oxime |
| | moxidectin |
| mite growth regulators | clofentezine |
| | cyromazine |
| | diflovidazin |
| | dofenapyn |
| | fluazuron |
| | flubenzimine |
| | flucycloxuron |
| | flufenoxuron |
| | hexythiazox |
| organochlorine acaricides | bromocyclen |
| | camphechlor |
| | DDT |
| | dienochlor |
| | endosulfan |
| | lindane |
| organophosphorus acaricides | |
| organophosphate acaricides | chlorfenvinphos |
| | crotoxyphos |
| | dichlorvos |
| | heptenophos |
| | mevinphos |
| | monocrotophos |
| | naled |
| | TEPP |
| | tetrachlorvinphos |
| organothiophosphate acaricides | amidithion |
| | amiton |
| | azinphos-ethyl |
| | azinphos-methyl |
| | azothoate |
| | benoxafos |
| | bromophos |
| | bromophos-ethyl |
| | carbophenothion |
| | chlorpyrifos |
| | chlorthiophos |
| | coumaphos |
| | cyanthoate |
| | demeton |
| | demeton-O |
| | demeton-S |
| | demeton-methyl |
| | demeton-O-methyl |
| | demeton-S-methyl |
| | demeton-S-methylsulphon |
| | dialifos |
| | diazinon |
| | dimethoate |
| | dioxathion |
| | disulfoton |
| | endothion |
| | ethion |
| | ethoate-methyl |
| | formothion |
| | malathion |
| | mecarbam |
| | methacrifos |
| | omethoate |
| | oxydeprofos |
| | oxydisulfoton |
| | parathion |
| | phenkapton |
| | phorate |
| | phosalone |
| | phosmet |
| | phostin |
| | phoxim |
| | pirimiphos-methyl |
| | prothidathion |
| | prothoate |
| | pyrimitate |
| | quinalphos |
| | quintiofos |
| | sophamide |
| | sulfotep |
| | thiometon |
| | triazophos |
| | trifenofos |
| | vamidothion |
| phosphonate acaricides | trichlorfon |
| phosphoramidothioate acaricides | isocarbophos |
| | methamidophos |
| | propetamphos |
| phosphorodiamide acaricides | dimefox |
| | mipafox |
| | schradan |
| organotin acaricides | azocyclotin |
| | cyhexatin |
| | fenbutatin oxide |
| | phostin |
| phenylsulfamide acaricides | dichlofluanid |
| phthalimide acaricides | dialifos |
| | phosmet |
| pyrazole acaricides | cyenopyrafen |
| | fenpyroximate |
| | pyflubumide |
| | tebufenpyrad |
| phenylpyrazole acaricides | acetoprole |
| | fipronil |
| | vaniliprole |
| pyrethroid acaricides | |
| pyrethroid ester acaricides | acrinathrin |
| | bifenthrin |
| | brofluthrinate |
| | cyhalothrin |
| | cypermethrin |
| | alpha-cypermethrin |
| | fenpropathrin |
| | fenvalerate |
| | flucythrinate |
| | flumethrin |
| | fluvalinate |
| | tau-fluvalinate |
| | permethrin |
| pyrethroid ether acaricides | halfenprox |
| pyrimidinamine acaricides | pyrimidifen |
| pyrrole acaricides | chlorfenapyr |
| quaternary ammonium acaricides | sanguinarine |
| quinoxaline acaricides | chinomethionat |
| | thioquinox |
| strobilurin acaricides | |
| methoxyacrylate strobilurin acaricides | bifujunzhi |
| | fluacrypyrim |
| | flufenoxystrobin |
| | pyriminostrobin |
| sulfite ester acaricides | aramite |
| | propargite |
| tetronic acid acaricides | spirodiclofen |
| tetrazine acaricides | clofentezine |
| | diflovidazin |
| thiazolidine acaricides | flubenzimine |
| | hexythiazox |
| thiocarbamate acaricides | fenothiocarb |
| thiourea acaricides | chloromethiuron |
| | diafenthiuron |
| unclassified acaricides | acequinocyl |
| | afoxolaner |
| | amidoflumet |
| | arsenous oxide |
| | clenpirin |
| | closantel |
| | crotamiton |
| | cycloprate |
| | cymiazole |
| | disulfiram |
| | etoxazole |
| | fenazaflor |
| | fenazaquin |
| | fluenetil |
| | fluralaner |
| | mesulfen |
| | MNAF |
| | nifluridide |
| | nikkomycins |
| | pyridaben |
| | sulfiram |
| | sulfluramid |
| | sulfur |
| | thuringiensin |
| | triarathene |
| CHEMOSTERILANTS | |
| | apholate |
| | bisazir |
| | busulfan |
| | diflubenzuron |
| | dimatif |
| | hemel |
| | hempa |
| | metepa |
| | methiotepa |
| | methyl apholate |
| | morzid |
| | penfluron |
| | tepa |
| | thiohempa |
| | thiotepa |
| | tretamine |
| | uredepa |
| INSECT REPELLENTS | |
| | acrep |
| | butopyronoxyl |
| | camphor |
| | d-camphor |
| | carboxide |
| | dibutyl phthalate |
| | diethyltoluamide |
| | dimethyl carbate |
| | dimethyl phthalate |
| | dibutyl succinate |
| | ethohexadiol |
| | hexamide |
| | icaridin |
| | methoquin-butyl |
| | methylneodecanamide |
| | 2-(octylthio)ethanol |
| | oxamate |
| | quwenzhi |
| | quyingding |
| | rebemide |
| | zengxiaoan |
| NEMATICIDES | |
| avermectin nematicides | abamectin |
| botanical nematicides | carvacrol |
| carbamate nematicides | benomyl |
| | carbofuran |
| | carbosulfan |
| | cloethocarb |
| oxime carbamate nematicides | alanycarb |
| | aldicarb |
| | aldoxycarb |
| | oxamyl |
| | tirpate |
| fumigant nematicides | carbon disulfide |
| | cyanogen |
| | 1,2-dichloropropane |
| | 1,3-dichloropropene |
| | dithioether |
| | methyl bromide |
| | methyl iodide |
| | sodium tetrathiocarbonate |
| organophosphorus nematicides | |
| organophosphate nematicides | diamidafos |
| | fenamiphos |
| | fosthietan |
| | phosphamidon |
| organothiophosphate nematicides | cadusafos |
| | chlorpyrifos |
| | dichlofenthion |
| | dimethoate |
| | ethoprophos |
| | fensulfothion |
| | fosthiazate |
| | heterophos |
| | isamidofos |
| | isazofos |
| | phorate |
| | phosphocarb |
| | terbufos |
| | thionazin |
| | triazophos |
| phosphonothioate nematicides | imicyafos |
| | mecarphon |
| unclassified nematicides | acetoprole |
| | benclothiaz |
| | chloropicrin |
| | dazomet |
| | DBCP |
| | DCIP |
| | fluazaindolizine |
| | fluensulfone |
| | furfural |
| | metam |
| | methyl isothiocyanate |
| | tioxazafen |
| | xylenols |

### Biorational Pesticides

Insecticides can be biorational or can also be known as biopesticides or biological pesticides. Biorational refers to any substance of natural origin (or man-made substances resembling those of natural origin) that has a detrimental or lethal effect on specific target pest(s), e.g., insects, weeds, plant diseases (including nematodes), and vertebrate pests, possess a unique mode of action, are non-toxic to man, domestic plants and animals, and have little or no adverse effects on wildlife and the environment.

Biorational insecticides (or biopesticides or biological pesticides) can be grouped as: (1) biochemicals (hormones, enzymes, pheromones and natural agents, such as insect and plant growth regulators), (2) microbial (viruses, bacteria, fungi, protozoa, and nematodes), or (3) Plant-Incorporated protectants (PIPs)—primarily transgenic plants, e.g., Bt soybean, including MON 87701 soybean.

Biopesticides, or biological pesticides, can broadly include agents manufactured from living microorganisms or a natural product and sold for the control of plant pests. Biopesticides can be: microorganisms, biochemicals, and semiochemicals. Biopesticides can also include peptides, proteins and nucleic acids such as double-stranded DNA, single-stranded DNA, double-stranded RNA, single-stranded RNA and hairpin DNA or RNA.

Bacteria, fungi, oomycetes, viruses and protozoa are all used for the biological control of insect pests. The most widely used microbial biopesticide is the insect pathogenic bacteria Bacillus thuringiensis (Bt), which produces a protein crystal (the Bt 6-endotoxin) during bacterial spore formation that is capable of causing lysis of gut cells when consumed by susceptible insects. Microbial Bt biopesticides consist of bacterial spores and 6-endotoxin crystals mass-produced in fermentation tanks and formulated as a sprayable product. Bt does not harm vertebrates and is safe to people, beneficial organisms and the environment. Thus, Bt sprays are a growing tactic for pest management on fruit and vegetable crops where their high level of selectivity and safety are considered desirable, and where resistance to synthetic chemical insecticides is a problem. Bt sprays have also been used on commodity crops such as maize, soybean and cotton, but with the advent of genetic modification of plants, farmers are increasingly growing Bt transgenic crop varieties.

Other microbial insecticides include products based on entomopathogenic baculoviruses. Baculoviruses that are pathogenic to arthropods belong to the virus family and possess large circular, covalently closed, and double-stranded DNA genomes that are packaged into nucleocapsids. More than 700 baculoviruses have been identified from insects of the orders Lepidoptera, Hymenoptera, and Diptera. Baculoviruses are usually highly specific to their host insects and thus, are safe to the environment, humans, other plants, and beneficial organisms. Over 50 baculovirus products have been used to control different insect pests worldwide.

At least 170 different biopesticide products based on entomopathogenic fungi have been developed for use against at least five insect and acarine orders in glasshouse crops, fruit and field vegetables as well as commodity crops. The majority of products are based on the ascomycetes Beauveria bassiana or Metarhizium anisopliae.

Plants produce a wide variety of secondary metabolites that deter herbivores from feeding on them. Some of these can be used as biopesticides. They include, for example, pyrethrins, which are fast-acting insecticidal compounds produced by Chrysanthemum cinerariaefolium. They have low mammalian toxicity but degrade rapidly after application. This short persistence prompted the development of synthetic pyrethrins (pyrethroids). The most widely used botanical compound is neem oil, an insecticidal chemical extracted from seeds of Azadirachta indica. Two highly active pesticides are available based on secondary metabolites synthesized by soil actinomycetes, but they have been evaluated by regulatory authorities as if they were synthetic chemical pesticides. Spinosad is a mixture of two macrolide compounds from Saccharopolyspora spinosa. It has a very low mammalian toxicity and residues degrade rapidly in the field. Farmers and growers used it widely following its introduction in 1997 but resistance has already developed in some important pests such as western flower thrips. Abamectin is a macrocyclic lactone compound produced by Streptomyces avermitilis. It is active against a range of pest species but resistance has developed to it also, for example, in tetranychid mites.

A semiochemical is a chemical signal produced by one organism that causes a behavioral change in an individual of the same or a different species. The most widely used semiochemicals for crop protection are insect sex pheromones, some of which can now be synthesized and are used for monitoring or pest control by mass trapping, lure-and-kill systems and mating disruption.

As used herein, “transgenic insecticidal trait” refers to a trait exhibited by a plant that has been genetically engineered to express a nucleic acid or polypeptide that is detrimental to one or more pests. In one embodiment, the plants of the present disclosure are resistant to attack and/or infestation from any one or more of the pests of the present disclosure. In one embodiment, the trait comprises the expression of vegetative insecticidal proteins (VIPs) from Bacillus thuringiensis, lectins and proteinase inhibitors from plants, terpenoids, cholesterol oxidases from Streptomyces spp., insect chitinases and fungal chitinolytic enzymes, bacterial insecticidal proteins and early recognition resistance genes. In another embodiment, the trait comprises the expression of a Bacillus thuringiensis protein that is toxic to a pest. In one embodiment, the Bt protein is a Cry protein (crystal protein). Bt crops include Bt corn, Bt cotton and Bt soy. Bt toxins can be from the Cry family (see, for example, Crickmore et al., 1998, Microbiol. Mol. Biol. Rev. 62: 807-812), which are particularly effective against Lepidoptera, Coleoptera and Diptera.

Bt Cry and Cyt toxins belong to a class of bacterial toxins known as pore-forming toxins (PFT) that are secreted as water-soluble proteins undergoing conformational changes in order to insert into, or to translocate across, cell membranes of their host. There are two main groups of PFT: (i) the α-helical toxins, in which α-helix regions form the trans-membrane pore, and (ii) the β-barrel toxins, that insert into the membrane by forming a β-barrel composed of Psheet hairpins from each monomer. See, Parker M W, Feil S C, “Pore-forming protein toxins: from structure to function,” Prog. Biophys. Mol. Biol. 2005 May; 88(1):91-142. The first class of PFT includes toxins such as the colicins, exotoxin A, diphtheria toxin and also the Cry three-domain toxins. On the other hand, aerolysin, α-hemolysin, anthrax protective antigen, cholesterol-dependent toxins as the perfringolysin O and the Cyt toxins belong to the β-barrel toxins. Id. In general, PFT producing-bacteria secrete their toxins and these toxins interact with specific receptors located on the host cell surface. In most cases, PFT are activated by host proteases after receptor binding inducing the formation of an oligomeric structure that is insertion competent. Finally, membrane insertion is triggered, in most cases, by a decrease in pH that induces a molten globule state of the protein. Id.

The development of transgenic crops that produce Bt Cry proteins has allowed the substitution of chemical insecticides by environmentally friendly alternatives. In transgenic plants the Cry toxin is produced continuously, protecting the toxin from degradation and making it reachable to chewing and boring insects. Cry protein production in plants has been improved by engineering cry genes with a plant biased codon usage, by removal of putative splicing signal sequences and deletion of the carboxy-terminal region of the protoxin. See, Schuler T H, et al., “Insect-resistant transgenic plants,” Trends Biotechnol. 1998; 16:168-175. The use of insect resistant crops has diminished considerably the use of chemical pesticides in areas where these transgenic crops are planted. See, Qaim M, Zilberman D, “Yield effects of genetically modified crops in developing countries,” Science. 2003 Feb. 7; 299(5608):900-2.

Known Cry proteins include: 6-endotoxins including but not limited to: the Cry1, Cry2, Cry3, Cry4, Cry5, Cry6, Cry7, Cry8, Cry9, Cry10, Cry11, Cry12, Cry13, Cry14, Cry15, Cry16, Cry17, Cry18, Cry19, Cry20, Cry21, Cry22, Cry23, Cry24, Cry25, Cry26, Cry27, Cry 28, Cry 29, Cry 30, Cry31, Cry32, Cry33, Cry34, Cry35, Cry36, Cry37, Cry38, Cry39, Cry40, Cry41, Cry42, Cry43, Cry44, Cry45, Cry 46, Cry47, Cry49, Cry 51, Cry52, Cry 53, Cry 54, Cry55, Cry56, Cry57, Cry58, Cry59. Cry60, Cry61, Cry62, Cry63, Cry64, Cry65, Cry66, Cry67, Cry68, Cry69, Cry70 and Cry71 classes of 6-endotoxin genes and the B. thuringiensis cytolytic cyt1 and cyt2 genes.

The use of Cry proteins as transgenic plant traits is well known to one skilled in the art and Cry-transgenic plants, including but not limited to, plants expressing: Cry1Ac, Cry1Ac+Cry2Ab, Cry1Ab, Cry1A.105, Cry1F, Cry1Fa2, Cry1F+Cry1Ac, Cry2Ab, Cry3A, mCry3A, Cry3Bbl, Cry34Abl, Cry35Abl, Vip3A, mCry3A, Cry9c and CBI-Bt have received regulatory approval. See, Sanahuja et al., “Bacillus thuringiensis: a century of research, development and commercial applications,” (2011) Plant Biotech Journal, April 9(3):283-300.

Pesticidal proteins also include VIP (vegetative insecticidal proteins) toxins. Entomopathogenic bacteria produce insecticidal proteins that accumulate in inclusion bodies or parasporal crystals (such as the aforementioned Cry and Cyt proteins), as well as insecticidal proteins that are secreted into the culture medium. Among the latter are the Vip proteins, which are divided into four families according to their amino acid identity. The Vip1 and Vip2 proteins act as binary toxins and are toxic to some members of the Coleoptera and Hemiptera. The Vip1 component is thought to bind to receptors in the membrane of the insect midgut, and the Vip2 component enters the cell, where it displays its ADP-ribosyltransferase activity against actin, preventing microfilament formation. Vip3 has no sequence similarity to Vip1 or Vip2 and is toxic to a wide variety of members of the Lepidoptera. Its mode of action has been shown to resemble that of the Cry proteins in terms of proteolytic activation, binding to the midgut epithelial membrane, and pore formation, although Vip3A proteins do not share binding sites with Cry proteins. The latter property makes them good candidates to be combined with Cry proteins in transgenic plants (Bacillus thuringiensis-treated crops [Bt crops]) to prevent or delay insect resistance and to broaden the insecticidal spectrum. There are commercially grown varieties of Bt cotton and Bt maize that express the Vip3Aa protein in combination with Cry proteins. For the most recently reported Vip4 family, no target insects have been found yet. See, Chakroun et al., “Bacterial Vegetative Insecticidal Proteins (Vip) from Entomopathogenic Bacteria,” Microbiol Mol Biol Rev. 2016 Mar. 2; 80(2):329-50. VIPs can be found in U.S. Pat. Nos. 5,877,012, 6,107,279 6,137,033, 7,244,820, 7,615,686, and 8,237,020 and the like.

Some currently registered PIPs are listed in Table 20.

TABLE 20
List of exemplary Plant-incorporated Protectants, which can
be combined with the recombinant plants of the disclosure
(e.g. incorporated into the genome of a soybean plant)

| | | Pesticide |
|---|---|---|
| Plant-Incorporated Protectants (PIPs) | Company and | Registration |
| Soybean | Trade Names | Numbers |
| Cry1Ac in Event 87701 Soybean PC Code | Monsanto | 524-594 |
| 006532 OECD Unique Identifier | Intacta | |
| Cry1A.105 and Cry2Ab2 in Event 87751 Soybean | Monsanto | 524-619 |
| PC Codes 006614, 006615 OECD Unique | | |
| Identifier MON-87751-7 | | |
| Cry1Ac × Cry1F in Event DAS 81419 Soybean | Mycogen | 68467-20 |
| PC Codes 006527, 006528 OECD Unique | Seeds/Dow Agro | |
| Identifier | | |
| DAS 81419 (Cry1Ac × Cry1F) | | |

### Herbicides

As aforementioned, agricultural compositions of the disclosure, which may be added to a recombinant plant as taught herein, are sometimes combined with (or contain) one or more herbicides.

In some embodiments, herbicidal compositions are applied to the plants and/or plant parts. In some embodiments, herbicidal compositions may be included in the compositions set forth herein and can be applied to a plant(s) or a part(s) thereof simultaneously or in succession, with other compounds.

Herbicides include 2,4-D, 2,4-DB, acetochlor, acifluorfen, alachlor, ametryn, atrazine, aminopyralid, benefin, bensulfuron, bensulide, bentazon, bicyclopyrone, bromacil, bromoxynil, butylate, carfentrazone, chlorimuron, chlorsulfuron, clethodim, clomazone, clopyralid, cloransulam, cycloate, DCPA, desmedipham, dicamba, dichlobenil, diclofop, diclosulam, diflufenzopyr, dimethenamid, diquat, diuron, DSMA, endothall, EPTC, ethalfluralin, ethofumesate, fenoxaprop, fluazifop-P, flucarbzone, flufenacet, flumetsulam, flumiclorac, flumioxazin, fluometuron, fluroxypyr, fomesafen, foramsulfuron, glufosinate, glyphosate, halosulfuron, hexazinone, imazamethabenz, imazamox, imazapic, imazaquin, imazethapyr, isoxaflutole, lactofen, linuron, MCPA, MCPB, mesotrione, metolachlor-s, metribuzin, indaziflam, metsulfuron, molinate, MSMA, napropamide, naptalam, nicosulfuron, norflurazon, oryzalin, oxadiazon, oxyfluorfen, paraquat, pelargonic acid, pendimethalin, phenmedipham, picloram, primisulfuron, prodiamine, prometryn, pronamide, propanil, prosulfuron, pyrazon, pyrithioac, quinclorac, quizalofop, rimsulfuron, S-metolachlor, sethoxydim, siduron, simazine, sulfentrazone, sulfometuron, sulfosulfuron, tebuthiuron, tembotrione, terbacil, thiazopyr, thifensulfuron, thiobencarb, topramezone, tralkoxydim, triallate, triasulfuron, tribenuron, triclopyr, trifluralin, and triflusulfuron.

In some embodiments, any one or more of the herbicides set forth herein may be utilized with any one or more of the plants or parts thereof set forth herein.

In some embodiments, any one or more of the herbicides set forth in the below Table 21 may be utilized.

TABLE 21
List of exemplary herbicides, which can be combined
with the recombinant plants of the disclosure

| | Herbicide | | |
|---|---|---|---|
| | Group | | |
| Site of Action | Number | Chemical Family | Herbicide |
| ACCase | 1 | Cyclohexanediones | Sethoxydim (Poast, |
| inhibitors | | | Poast Plus) |
| | | | Clethodim (Select, |
| | | | Select Max, Arrow) |
| | | Aryloxyphenoxypropionates | Fluazifop (Fusilade DX, |
| | | | component in Fusion) |
| | | | Fenoxaprop (Puma, |
| | | | component in Fusion) |
| | | | Quizalofop (Assure II, |
| | | | Targa) |
| | | Phenylpyrazolins | Pinoxaden (Axial XL) |
| ALS inhibitors | 2 | Imidazolinones | Imazethapyr (Pursuit) |
| | | | Imazamox (Raptor) |
| | | Sulfonylureas | Chlorimuron (Classic) |
| | | | Halosulfuron (Permit, |
| | | | Sandea) |
| | | | Iodosulfuron |
| | | | (component in Autumn |
| | | | Super) |
| | | | Mesosulfuron (Osprey) |
| | | | Nicosulfuron (Accent Q) |
| | | | Primisulfuron (Beacon) |
| | | | Prosulfuron (Peak) |
| | | | Rimsulfuron (Matrix, |
| | | | Resolve) |
| | | | Thifensulfuron |
| | | | (Harmony) |
| | | | Tribenuron (Express) |
| | | | Triflusulfuron (UpBeet) |
| | | Triazolopyrimidine | Flumetsulam (Python) |
| | | | Cloransulam-methyl |
| | | | (FirstRate) |
| | | | Pyroxsulam (PowerFlex |
| | | | HL) |
| | | | Florasulam (component |
| | | | in Quelex) |
| | | Sulfonylaminocarbonyltriazolinones | Propoxycarbazone |
| | | | (Olympus) |
| | | | Thiencarbazone-methyl |
| | | | (component in |
| | | | Capreno) |
| Microtubule | 3 | Dinitroanilines | Trifluralin (many |
| inhibitors (root | | | names) |
| inhibitors) | | | Ethalfluralin (Sonalan) |
| | | | Pendimethalin |
| | | | (Prowl/Prowl H₂O) |
| | | Benzamide | Pronamide (Kerb) |
| Synthetic auxins | 4 | Arylpicolinate | Halauxifen (Elevore, |
| | | | component in Quelex) |
| | | Phenoxy acetic acids | 2,4-D (Enlist One, |
| | | | others) |
| | | | 2,4-DB (Butyrac 200, |
| | | | Butoxone 200) |
| | | | MCPA |
| | | Benzoic acids | Dicamba (Banvel, |
| | | | Clarity, DiFlexx, |
| | | | Engenia, XtendiMax; |
| | | | component in Status) |
| | | Pyridines | Clopyralid (Stinger) |
| | | | Fluroxypyr (Starane |
| | | | Ultra) |
| Photosystem II | 5 | Triazines | Atrazine |
| inhibitors | | | Simazine (Princep, Sim- |
| | | | Trol) |
| | | Triazinone | Metribuzin (Metribuzin, |
| | | | others) |
| | | | Hexazinone (Velpar) |
| | | Phenyl-carbamates | Desmedipham (Betenex) |
| | | | Phenmedipham |
| | | | (component in Betamix) |
| | 6 | Uracils | Terbacil (Sinbar) |
| | | Benzothiadiazoles | Bentazon (Basagran, |
| | | | others) |
| | | Nitriles | Bromoxynil (Buctril, |
| | | | Moxy, others) |
| | 7 | Phenylureas | Linuron (Lorox, Linex) |
| Lipid synthesis | 8 | Thiocarbamates | EPTC (Eptam) |
| inhibitor | | | |
| EPSPS inhibitor | 9 | Organophosphorus | Glyphosate |
| Glutamine | 10 | Organophosphorus | Glufosinate (Liberty, |
| synthetase | | | Rely) |
| inhibitor | | | |
| Diterpene | 13 | Isoxazolidinone | Clomazone (Command) |
| biosynthesis | | | |
| inhibitor | | | |
| (bleaching) | | | |
| Protoporphyrinogen | 14 | Diphenylether | Acifluorfen (Ultra |
| oxidase | | | Blazer) |
| inhibitors (PPO) | | | Fomesafen (Flexstar, |
| | | | Reflex) |
| | | | Lactofen (Cobra, |
| | | | Phoenix) |
| | | N-phenylphthalimide | Flumiclorac (Resource) |
| | | | Flumioxazin (Valor, |
| | | | Valor EZ, Rowel) |
| | | Aryl triazolinone | Sulfentrazone |
| | | | (Authority, Spartan) |
| | | | Carfentrazone (Aim) |
| | | | Fluthiacet-methyl |
| | | | (Cadet) |
| | | Pyrazoles | Pyraflufen-ethyl (Vida) |
| | | Pyrimidinedione | Saflufenacil (Sharpen) |
| Long-chain fatty | 15 | Acetamides | Acetochlor (Harness, |
| acid inhibitors | | | Surpass NXT, |
| | | | Breakfree NXT, |
| | | | Warrant) |
| | | | Dimethenamid-P |
| | | | (Outlook) |
| | | | Metolachlor (Parallel) |
| | | | Pyroxasulfone (Zidua, |
| | | | Zidua SC) |
| | | | s-metolachlor (Dual |
| | | | Magnum, Dual II |
| | | | Magnum, Cinch) |
| | | | Flufenacet (Define) |
| Specific site | 16 | Benzofuranes | Ethofumesate (Nortron) |
| unknown | | | |
| Auxin transport | 19 | Semicarbazone | diflufenzopyr |
| inhibitor | | | (component in Status) |
| Photosystem I | 22 | Bipyridiliums | Paraquat (Gramoxone, |
| inhibitors | | | Parazone) |
| | | | Diquat (Reglone) |
| 4-HPPD | 27 | Isoxazole | Isoxaflutole (Balance |
| inhibitors | | Pyrazole | Flexx) |
| (bleaching) | | Pyrazolone | Pyrasulfotole |
| | | Triketone | (component in Huskie) |
| | | | Topramezone |
| | | | (Armezon/Impact) |
| | | | Bicyclopyrone |
| | | | (component in Acuron) |
| | | | Mesotrione (Callisto) |
| | | | Tembotrione (Laudis) |

### Fungicides

As aforementioned, agricultural compositions of the disclosure, which may be added to a recombinant plant as taught herein, are sometimes combined with (or contain) one or more fungicides.

In some embodiments, fungicidal compositions may be included in the compositions set forth herein, and can be applied to a plant(s) or a padt(s) thereof simultaneously or in succession, with other compounds. The fungicides include azoxystrobin, captan, carboxin, ethaboxam, fludioxonil, mefenoxam, fludioxonil, thiabendazole, thiabendaz, ipconazole, mancozeb, cyazofamid, zoxamide, metalaxyl, PCNB, metaconazole, pyraclostrobin, Bacillus subtilis strain QST 713, sedaxane, thiamethoxam, fludioxonil, thiram, tolclofos-methyl, trifloxystrobin, Bacillus subtilis strain MBI 600, pyraclostrobin, fluoxastrobin, Bacillus pumilus strain QST 2808, chlorothalonil, copper, flutriafol, fluxapyroxad, mancozeb, gludioxonil, penthiopyrad, triazole, propiconaozole, prothioconazole, tebuconazole, fluoxastrobin, pyraclostrobin, picoxystrobin, qols, tetraconazole, trifloxystrobin, cyproconazole, flutriafol, SDHI, EBDCs, sedaxane, MAXIM QUATTRO (gludioxonil, mefenoxam, azoxystrobin, and thiabendaz), RAXIL (tebuconazole, prothioconazole, metalaxyl, and ethoxylated tallow alkyl amines), and benzovindiflupyr.

In some embodiments, any one or more of the fungicides set forth herein may be utilized with any one or more of the plants or parts thereof set forth herein.

### Nematicides

As aforementioned, agricultural compositions of the disclosure, which may be added to a recombinant plant as taught herein, are sometimes combined with (or contain) one or more nematicides.

In some embodiments, nematicidal compositions may be included in the compositions set forth herein, and can be applied to a plant(s) or a part(s) thereof simultaneously or in succession, with other compounds. The nematicides may be selected from D-D, 1,3-dichloropropene, ethylene dibromide, 1,2-dibromo-3-chloropropane, methyl bromide, chloropicrin, metam sodium, dazomet, methylisothiocyanate, sodium tetrathiocarbonate, aldicarb, aldoxycarb, carbofuran, oxamyl, ethoprop, fenamiphos, cadusafos, fosthiazate, terbufos, fensulfothion, phorate, DiTera, clandosan, sincocin, methyl iodide, propargyl bromide, 2,5-dihydroxymethyl-3,4-dihydroxypyrrolidine (DMDP), any one or more of the avermectins, sodium azide, furfural, Bacillusfirmus, abamectrin, thiamethoxam, fludioxonil, clothiandin, salicylic acid, and benzo-(1,2,3)-thiadiazole-7-carbothioic acid S-methyl ester.

In some embodiments, any one or more of the nematicides set forth herein may be utilized with any one or more of the plants or parts thereof set forth herein.

In some embodiments, any one or more of the nematicides, fungicides, herbicides, insecticides, and/or pesticides set forth herein may be utilized with any one or more of the plants or parts thereof set forth herein.

### Fertilizers, Nitrogen Stabilizers, and Urease Inhibitors

As aforementioned, agricultural compositions of the disclosure, which may be added to a recombinant plant as taught herein, are sometimes combined with (or contain) one or more of a: fertilizer, nitrogen stabilizer, or urease inhibitor.

Fertilizers include anhydrous ammonia, urea, ammonium nitrate, and urea-ammonium nitrate (UAN) compositions, among many others.

Nitrogen stabilizers include nitrapyrin, 2-chloro-6-(trichloromethyl) pyridine, N-SERVE 24, INSTINCT, dicyandiamide (DCD).

Further, stabilized forms of fertilizer can be used. For example, a stabilized form of fertilizer is SUPER U, containing 46% nitrogen in a stabilized, urea-based granule, SUPERU contains urease and nitrification inhibitors to guard from dentrification, leaching, and volatilization. Stabilized and targeted foliar fertilizer such as NITAMIN may also be used herein.

Slow- or controlled-release fertilizer that may be used herein entails: A fertilizer containing a plant nutrient in a form which delays its availability for plant uptake and use after application, or which extends its availability to the plant significantly longer than a reference ‘rapidly available nutrient fertilizer’ such as ammonium nitrate or urea, ammonium phosphate or potassium chloride. Such delay of initial availability or extended time of continued availability may occur by a variety of mechanisms. These include controlled water solubility of the material by semi-permeable coatings, occlusion, protein materials, or other chemical forms, by slow hydrolysis of water-soluble low molecular weight compounds, or by other unknown means.

Stabilized nitrogen fertilizer that may be used herein entails: A fertilizer to which a nitrogen stabilizer has been added. A nitrogen stabilizer is a substance added to a fertilizer which extends the time the nitrogen component of the fertilizer remains in the soil in the urea-N or ammoniacal-N form.

Nitrification inhibitor that may be used herein entails: A substance that inhibits the biological oxidation of ammoniacal-N to nitrate-N. Some examples include: (1) 2-chloro-6-(trichloromethyl-pyridine), common name Nitrapyrin, manufactured by Dow Chemical; (2) 4-amino-1,2,4-6-triazole-HCl, common name ATC, manufactured by Ishihada Industries; (3) 2,4-diamino-6-trichloro-methyltriazine, common name CI-1580, manufactured by American Cyanamid; (4) Dicyandiamide, common name DCD, manufactured by Showa Denko; (5) Thiourea, common name TU, manufactured by Nitto Ryuso; (6) 1-mercapto-1,2,4-triazole, common name MT, manufactured by Nippon; (7) 2-amino-4-chloro-6-methyl-pyramidine, common name AM, manufactured by Mitsui Toatsu; (8) 3,4-dimethylpyrazole phosphate (DMPP), from BASF; (9) 1-amide-2-thiourea (ASU), from Nitto Chemical Ind.; (10) Ammoniumthiosulphate (ATS); (11) 1H-1,2,4-triazole (HPLC); (12) 5-ethylene oxide-3-trichloro-methly 1,2,4-thiodiazole (Terrazole), from Olin Mathieson; (13) 3-methylpyrazole (3-MP); (14) 1-carbamoyle-3-methyl-pyrazole (CMP); (15) Neem; and (16) DMPP.

Urease inhibitor that may be used herein entails: A substance that inhibits hydrolytic action on urea by the enzyme urease. Thousands of chemicals have been evaluated as soil urease inhibitors (Kiss and Simihaian, 2002). However, only a few of the many compounds tested meet the necessary requirements of being non toxic, effective at low concentration, stable, and compatible with urea (solid and solutions), degradable in the soil and inexpensive. They can be classified according to their structures and their assumed interaction with the enzyme urease (Watson, 2000, 2005). Four main classes of urease inhibitors have been proposed: (a) reagents which interact with the sulphydryl groups (sulphydryl reagents), (b) hydroxamates, (c) agricultural crop protection chemicals, and (d) structural analogues of urea and related compounds. N-(n-Butyl) thiophosphoric triamide (NBPT), phenylphosphorodiamidate (PPD/PPDA), and hydroquinone are probably the most thoroughly studied urease inhibitors (Kiss and Simihaian, 2002). Research and practical testing has also been carried out with N-(2-nitrophenyl) phosphoric acid triamide (2-NPT) and ammonium thiosulphate (ATS). The organo-phosphorus compounds are structural analogues of urea and are some of the most effective inhibitors of urease activity, blocking the active site of the enzyme (Watson, 2005).

### Examples of Genetically Modified Soybean Plants

The recombinant plants taught herein, which contain expression constructs for expressing milk proteins, may be added to or “stacked” into the genome of already genetically modified plants, see Table 22. For example, the following genetically modified plant events, which have been approved in one or more countries, can be utilized as a base soybean variety, into which the milk protein expression events can be added/stacked.

TABLE 22
Soybean Traits, which can be stacked with milk protein expression events
Glycine max L. Soybean

| Event | Company | Description |
|---|---|---|
| A2704-12, | Bayer CropScience | Glufosinate ammonium herbicide |
| A2704-21, | (Aventis | tolerant soybean produced by |
| A5547-35 | CropScience | inserting a modified |
| | (AgrEvo)) | phosphinothricin acetyltransferase |
| | | (PAT) encoding gene from the soil |
| | | bacterium Streptomyces |
| | | viridochromogenes. |
| A5547-127 | Bayer CropScience | Glufosinate ammonium herbicide |
| | (Aventis | tolerant soybean produced by |
| | CropScience | inserting a modified |
| | (AgrEvo)) | phosphinothricin acetyltransferase |
| | | (PAT) encoding gene from the soil |
| | | bacterium Streptomyces |
| | | viridochromogenes. |
| BPS-CV127-9 | BASF Inc. | The introduced csr1-2 gene from |
| | | Arabidopsis thaliana encodes an |
| | | acetohydroxyacid synthase protein |
| | | that confers tolerance to |
| | | imidazolinone herbicides due to a |
| | | point mutation that results in a |
| | | single amino acid substitution in |
| | | which the serine residue at position |
| | | 653 is replaced by asparagine |
| | | (S653N). |
| DP-305423 | Pioneer Hi-Bred | High oleic acid soybean produced |
| | International Inc. | by inserting additional copies of a |
| | | portion of the omega 6 desaturase |
| | | encoding gene, gm-fad2-1 |
| | | resulting in silencing of the |
| | | endogenous omega-6 desaturase |
| | | gene (FAD2-1). |
| DP356043 | Pioneer Hi-Bred | Soybean event with two herbicide |
| | International Inc. | tolerance genes: glyphosate N- |
| | | acetlytransferase, which detoxifies |
| | | glyphosate, and a modified |
| | | acetolactate synthase (ALS) gene |
| | | which is tolerant to ALS-inhibiting |
| | | herbicides. |
| G94-1, | DuPont Canada | High oleic acid soybean produced |
| G94-19, | Agricultural | by inserting a second copy of the |
| G168 | Products | fatty acid desaturase (Gm Fad2-1) |
| | | encoding gene from soybean, |
| | | which resulted in “silencing” of |
| | | the endogenous host gene. |
| GTS 40-3-2 | Monsanto Company | Glyphosate tolerant soybean |
| | | variety produced by inserting a |
| | | modified 5-enolpyruvylshikimate- |
| | | 3- phosphate synthase (EPSPS) |
| | | encoding gene from the soil |
| | | bacterium Agrobacterium |
| | | tumefaciens. |
| GU262 | Bayer CropScience | Glufosinate ammonium herbicide |
| | (Aventis | tolerant soybean produced by |
| | CropScience | inserting a modified |
| | (AgrEvo)) | phosphinothricin acetyltransferase |
| | | (PAT) encoding gene from the soil |
| | | bacterium Streptomyces |
| | | viridochromogenes. |
| MON87701 | Monsanto Company | Resistance to Lepidopteran pests |
| | | of soybean including velvetbean |
| | | caterpillar (Anticarsia gemmatalis) |
| | | and soybean looper (Pseudoplusia |
| | | includens). |
| MON87701 × | Monsanto Company | Glyphosate herbicide tolerance |
| MON89788 | | through expression of the EPSPS |
| | | encoding gene from A. tumefaciens |
| | | strain CP4, and resistance to |
| | | Lepidopteran pests of soybean |
| | | including velvetbean caterpillar |
| | | (Anticarsia gemmatalis) and |
| | | soybean looper (Pseudoplusia |
| | | includens) via expression of the |
| | | Cry1Ac encoding gene from B. |
| | | thuringiensis. |
| MON89788 | Monsanto Company | Glyphosate-tolerant soybean |
| | | produced by inserting a modified |
| | | 5-enolpyruvylshikimate-3- |
| | | phosphate synthase (EPSPS) |
| | | encoding aroA (epsps) gene from |
| | | Agrobacterium tumefaciens CP4. |
| OT96-15 | Agriculture & | Low linolenic acid soybean |
| | Agri-Food | produced through traditional cross- |
| | Canada | breeding to incorporate the novel |
| | | trait from a naturally occurring |
| | | fan1 gene mutant that was selected |
| | | for low linolenic acid. |
| W62, W98 | Bayer CropScience | Glufosinate ammonium herbicide |
| | (Aventis | tolerant soybean produced by |
| | CropScience | inserting a modified |
| | (AgrEvo)) | phosphinothricin acetyltransferase |
| | | (PAT) encoding gene from the soil |
| | | bacterium Streptomyces |
| | | hygroscopicus. |

### Soybean Planting Density

Plant spacing in the field is very important and plays a significant role in determining plant growth and development. Plant spacing should be thought of as existing in two directions: 1) within row spacing and 2) between row spacing. At a given plant population, as row spacing decreases the plant spacing within the row increases and results in a more equidistant plant spacing. At a fixed row width, as plant population increases the plant spacing within the row decreases and interplant competition increases. Obviously, both factors can be adjusted to provide optimal plant spacing and typically plant population increases as row spacing decreases. The expectation would be that narrow row spacing and increased plant population would improve light interception and that would then have a cascade effect, increasing crop growth rate and potentially improving seed production. An important consideration would be that as plant population increases the available source (light) per plant decreases. Since there is good evidence that soybean is already source limited, any more reduction on source capacity would come at a cost of yield per plant.

A low plant population can be competitive with higher plant populations as long as the plant population is uniformly distributed in the field. At lower populations interplant competition is reduced and each individual plant has a greater opportunity to produce more seeds. Research has shown that photosynthetic rate is greater and maintained longer in response to larger sink size (seed number) by increased protein levels in leaves and reduced leaf senescence of older leaves.

The risks associated with reduced plant populations is that pods form closer to the ground increasing harvest losses and the plant must maintain a high growth rate and leaf area throughout the season to remain competitive with higher populations. Soybean is able to compensate for poor stands by producing more branches resulting in yields comparable to higher plant populations. However, low plant populations may result in lower pod height, lodging of lateral branches, and higher weed populations, all of which may result in harvest losses.

An important consideration when selecting the correct seeding rate is that seeds planted per acre will not be the same as plants per acre. Not all of the seed that is planted will germinate or grow to reach plant maturity. The proper way to think about seeding rate is the number of plants that need to reach maturity to maximize yield. The term plant population refers to the number of soybean plants per acre. Seeding rate or planting rate refers to the number of soybean seeds planted per acre to attain a certain plant population. Most farmers find it easier and more reliable to count number of seeds per foot of row or seeds per acre instead of pounds or bushels per acre. Seed size varies from one variety to another; therefore one variety may have fewer seeds per pound than another variety. Plant population depends on seedbed conditions and planter settings. Planting into a poor seedbed, use of poor quality seed, inaccurate planter adjustment, planting too fast, soil crusting, soil moisture extremes, and environmentally induced plant injuries such as herbicide drift, pathogens, insects, hail, or frost all reduce plant population requiring a greater seeding rate. For example, if 120,000 plants per acre is the plant population needed at harvest to maximize yield then more than 120,000 seeds will need to be planted to compensate for plant loss throughout the season. Increasing the seeding rate by 15 to 30% over desired final plant stand to compensate for plant loss is a good estimate.

Agronomic studies over the past 20 years have often shown that the chance of getting yield increases as plant population moves above 100,000 plants per acre is small. However, there are cases where 100,000 plants per acre is not enough. Nevertheless, most producers have resisted lowering their planting rates accordingly. In part, this is because current university recommendations often include planting 140,000 to 225,000 seeds per acre, with adjustments depending on row spacing and planting

To answer questions about whether current seeding rates are appropriate, numerous studies were initiated in 2003 to update university and extension service recommendations. The data points to a need for 100,000 healthy uniform distributed plants per acre. To achieve such, a seeding rate of 125,000 to 140,000 viable seeds per acre (need to be increased if the germination is below 90%) can be utilized. However, the rate can be modified as needed. For example, with wet seedbed conditions or with a lot of residues (e.g., reduced tillage practices), then it may be advantageous to increase seeding rate to 140,000 seeds per acre. A decision tool is available from several universities and extension service providers to help target the final stand of 100,000 plants per acre. Accordingly, for a 15 inch row spacing, one could utilize 125,000 to 140,000 seeds per acre. For a 30 inch row spacing, one could utilize 125,000 to 140,000 seeds per acre.

Improved planter technology and increased seed quality have resulted in more accurate plant populations and stand establishment that are only slightly reduced from the seeding rate, and do not require over seeding. (See the Iowa State Extension Service's detailed recommendations and data on soybean planting density, from which the aforementioned section was obtained, on the world wide web at crops.extension.iastate.edu/encyclopedia/soybean-plant-population).

### Soybean Varieties

Soybean maturity groups (MG) are latitudinal zones developed to define where a soybean genetic package best fits based on photoperiod and temperature. MGs ranging from 000 for the very early maturing varieties to 9 for the later. Gradations within MGs are also commonly noted by adding a decimal to the MG number. A variety is classified to a specific MG according to the length of period from planting to maturity. All MGs are applicable to the disclosure herein and can be utilized to produce recombinant milk proteins.

### Common Soybean Varieties

As exemplary embodiments, the following tables illustrate various MG 4 and MG 5 soybean varieties that are applicable for the genomic architecture edits of the present disclosure, which can create robust biofactories to produce recombinant proteins, e.g., recombinant milk proteins.

The soybean varieties in the following tables attempt to group various varieties that are genetically identical, but which have various branding designations. (See University of Arkansas Extension Service research publication: “2021 Soybean Cross Reference Guide.”).

### Fatty Acid Modulation

In some embodiments, the present disclosure teaches methods for modulating fatty acid profiles of plant-based products to better mimic their intended animal-based counterpart. Products with modulated fatty acid profiles are also envisioned. In some embodiments, fatty acid modulation is achieved by blending of one or more plant-based oil/fat to adjust the fatty acid profile of the original product.

In some embodiments, a difference in a fatty acid profile can result in a difference in flavor and texture. For example, milk fat is composed of triacylglycerides from a wide range of fatty acids. The fatty acids can contribute to the melting properties, mouthfeel, and/or flavor of a food product. Short chain fatty acids (SCFA) impart the characteristic flavor to dairy products with bovine lipids being higher in C4:0 (butyric acid) while caprine lipids have relatively higher levels of C6:0 (caproic acid), C8:0 (caprylic acid), and C:10 (capric). Milk fats are the primary source of SCFA in our diets. The melting properties of a triglyceride are correlated with chain length and degree of saturation of each of the three fatty acids bonded to the glycerine backbone. Devi and Khatkar, 2017, incorporated by reference herein, provides a list of the melting properties of individual fatty acids as well as some covalently bonded compounds with those fatty acids. Knothe and Dunn, 2009, incorporated by reference herein, shows some melting properties of butter and selected vegetable lipids.

Triacylglycerides with lower melting points, typical of short chain and unsaturated fatty acids, release their flavors quickly in the mouth. Flavor release of cheeses made with fewer lower melting fatty acids can be improved by eating from a warm temperature (e.g. room or cooked). Lower melting fats have less of a creamy texture than those with a melting temperature than about 30 C. Fats that contain a significant amount triacylglycerides with a melt point greater than about 40 C are described as waxy. Those with a melting point of 10 C or less are often described as oily.

Milkfats have diverse fatty acid profiles and are higher in SCFA than plant fats and the milk triglycerides are melted at body temperature. Palm oil has a similar melt point to fat, but two fatty acids, C16:0 and C18:1, represent 85% of the fatty acid profile and C18:2 contributes another 10%. Palm oil is used to replace milkfat in a number of applications including frozen desserts. While its texture is close to milkfat, it lacks the flavor release of butter. Coconut oil has a lower melt point than milkfat and is also used in some dairy alternatives. The melting profile of coconut oil is much steeper than milkfat and it does not release flavors as quickly as milkfat. The high level of C12:0 can impart a soapy flavor to products. Plant based oils (e.g. soy, sunflower, canola, etc.) have a high level of unsaturated fatty acids that impart a very low melting point and are susceptible to oxidative rancidity. These lipids are preferred for their low cost but are generally considered inferior to harder fats in most food applications. Hydrogenation is used to impart properties that are closer to animal fats.

In some embodiments, blending lipids can extend expensive lipids and/or impart properties different from a single fat source. Butter can be blended with vegetable oils to produce a spreadable product from the refrigerator. Hydrogenation of oils or fractionation to separate high melt fractions for shortening and frying raises the melt point of oils. For example, a soybean oil with high levels of SCFA combined with other lipid sources would give an improved fat for use in dairy alternatives.

In some embodiments, a composition does not have a fatty acid profile comprising: palmitic acid (16:0) at about 5-15%, stearic acid (18:0) at about 1-5%, oleic acid (18:1) at about 15-20%, linoleic acid (18:2) at about 50-60%, and linolenic acid (18:3) at about 10-15% wherein the fatty acid profile represents (wt/wt) percent over total fatty acids. In some embodiments, a composition does not have a fatty acid profile comprising: palmitic acid (16:0) at about 10%, stearic acid (18:0) at about 4%, oleic acid (18:1) at about 18%, linoleic acid (18:2) at about 55%, and linolenic acid (18:3) at about 13% wherein the fatty acid profile represents (wt/wt) percent over total fatty acids.

In some embodiments, a composition comprises a fatty acid profile comprising palmitic acid (16:0) at about 23-32% wherein the fatty acid profile represents (wt/wt) percent over total fatty acids. In some embodiments, a composition comprises a fatty acid profile comprising palmitic acid (16:0) at about 13-42% wherein the fatty acid profile represents (wt/wt) percent over total fatty acids. In some embodiments, a composition comprises a fatty acid profile comprising stearic acid (18:0) at about 21-26% wherein the fatty acid profile represents (wt/wt) percent over total fatty acids. In some embodiments, a composition comprises a fatty acid profile comprising stearic acid (18:0) at about 11-36% wherein the fatty acid profile represents (wt/wt) percent over total fatty acids. In some embodiments, a composition comprises a fatty acid profile comprising oleic acid (18:1) at about 17-27% wherein the fatty acid profile represents (wt/wt) percent over total fatty acids. In some embodiments, a composition comprises a fatty acid profile comprising oleic acid (18:1) at about 7-37% wherein the fatty acid profile represents (wt/wt) percent over total fatty acids. In some embodiments, a composition comprises a fatty acid profile comprising linoleic acid (18:2) at about 0.5-3.1% wherein the fatty acid profile represents (wt/wt) percent over total fatty acids. In some embodiments, a composition comprises a fatty acid profile comprising linoleic acid (18:2) at about 0.1-13.1% wherein the fatty acid profile represents (wt/wt) percent over total fatty acids.

In some embodiments, a composition comprises a fatty acid profile comprising: palmitic acid (16:0) at about 23-32%; stearic acid (18:0) at about 21%-26%; oleic acid (18:1) at about 17-27%; and/or linoleic acid (18:2) at about 0.5-3.1%, wherein the fatty acid profile represents (wt/wt) percent over total fatty acids. In some embodiments, a composition comprises a fatty acid profile comprising one or more of: palmitic acid (16:0) at about 23-32%; stearic acid (18:0) at about 21%-26%; oleic acid (18:1) at about 17-27%; and linoleic acid (18:2) at about 0.5-3.1%, wherein the fatty acid profile represents (wt/wt) percent over total fatty acids. In some embodiments, a composition comprises a fatty acid profile comprising palmitic acid (16:0) at about 13-42%; stearic acid (18:0) at about 11-36%; oleic acid (18:1) at about 7-37%; and linoleic acid (18:2) at about 0.01-13.1%, wherein the fatty acid profile represents (wt/wt) percent over total fatty acids. In some embodiments, a composition comprises a fatty acid profile comprising palmitic acid (16:0) at about 28%; stearic acid (18:0) at about 11%; oleic acid (18:1) at about 22%; and linoleic acid (18:2) at about 2.5%, wherein the fatty acid profile represents (wt/wt) percent over total fatty acids.

In some embodiments, a composition comprises a fatty acid profile comprising any one or more of: C4:0 fatty acids at about 1%-7%; C6:0 fatty acids at about 0.5-6%; C8:0 fatty acid at about 0.5%-4.5%; C10:0 fatty acids at about 0.4%-6%; C12:0 fatty acids at about 0.4%-6.5%; C14:0 fatty acids at about 7%-13%; C16:0 fatty acids at about 26%-32%; C16:1 fatty acid at about 0.2%-4.2%; C18:0 fatty acids at about 14%-21%; C18:1 fatty acids at about 18%-26%; and/or C18:2 fatty acids at about 0.5%-7% wherein the fatty acid profile represents (wt/wt) percent over total fatty acids. In some embodiments, a composition comprises a fatty acid profile comprising any one or more of: C4:0 fatty acids at about 0.01%-17%; C6:0 fatty acids at about 0.01-16%; C8:0 fatty acid at about 0.01-14.5%; C10:0 fatty acids at about 0.01%-16%; C12:0 fatty acids at about 0.01%-16.5%; C14:0 fatty acids at about 0.1-23%; C16:0 fatty acids at about 16%-42%; C16:1 fatty acid at about 0.01-14.2%; C18:0 fatty acids at about 4-31%; C18:1 fatty acids at about 8-36%; and/or C18:2 fatty acids at about 0.01-17% wherein the fatty acid profile represents (wt/wt) percent over total fatty acids.

In some embodiments, a composition comprises a fatty acid profile comprising C4:0 fatty acids at about 1%-7%; C6:0 fatty acids at about 0.5-6%; C8:0 fatty acids at about 0.5%-4.5%; C10:0 fatty acids at about 0.4%-6%; C12:0 fatty acids at about 0.4%-6.5%; C14:0 fatty acids at about 7%-13%; C16:0 fatty acids at about 26%-32%; C16:1 fatty acids at about 0.2%-4.2%; C18:0 fatty acids at about 14%-21%; C18:1 fatty acids at about 18%-26%; and/or C18:2 fatty acids at about 0.5%-7% wherein the fatty acid profile represents (wt/wt) percent over total fatty acids.

In some embodiments, a composition comprises a fatty acid profile comprising: C4:0 fatty acids at about 0.01%-17%; C6:0 fatty acids at about 0.01-16%; C8:0 fatty acids at about 0.01%-14.5%; C10:0 fatty acids at about 0.01%-16%; C12:0 fatty acids at about 0.01%-16.5%; C14:0 fatty acids at about 0.1%-23%; C16:0 fatty acids at about 16%-42%; C16:1 fatty acids at about 0.01%-14.2%; C18:0 fatty acids at about 4%-31%; C18:1 fatty acids at about 8%-36%; and/or C18:2 fatty acids at about 0.01%-17% wherein the fatty acid profile represents (wt/wt) percent over total fatty acids.

In some embodiments, a composition comprises a fatty acid profile comprising: C4:0 fatty acids at about 4.34%; C6:0 fatty acids at about 2.70%; C8:0 fatty acids at about 1.60%; C10:0 fatty acids at about 3.40%; C12:0 fatty acids at about 3.48%; C14:0 fatty acids at about 10.00%; C16:0 fatty acids at about 29.20%; C16:1 fatty acids at about 1.29%; C18:0 fatty acids at about 17.47%; C18:1 fatty acids at about 22.84%; and/or C18:2 fatty acids at about 3.67% wherein the fatty acid profile represents (wt/wt) percent over total fatty acids.

In some embodiments, any of the aforementioned fatty acids can be at a percent of from about 0%, 0.2%, 0.4%, 0.6%, 0.8%, 1%, 1.2%, 1.4%, 1.6%, 1.8%, 2%, 2.2%, 2.4%, 2.6%, 2.8%, 30%, 3.20%, 3.40%, 3.60%, 3.80%, 40%, 4.20%, 4.40%, 4.60%, 4.80%, 50%, 5.2%, 5.4%, 5.6%, 5.80%, 60%, 6.20%, 6.40%, 6.60%, 6.80%, 70%, 7.20%, 7.40%, 7.60%, 7.80%, 8%, 8.2%, 8.4%, 8.6%, 8.8%, 9%, 9.2%, 9.4%, 9.6%, 9.8%, 10%, 10.2%, 10.4%, 10.6%, 10.8%, 11%, 11.2%, 11.4%, 11.6%, 11.8%, 120%, 12.2%, 12.4%, 12.6%, 12.8%, 13%, 13.2%, 13.4%, 13.6%, 13.8%, 14%, 14.2%, 14.4%, 14.6%, 14.8%, 15%, 15.2%, 15.4%, 15.6%, 15.8%, 16%, 16.2%, 16.4%, 16.6%, 16.8%, 17%, 17.2%, 17.4%, 17.6%, 17.8%, 18%, 18.2%, 18.4%, 18.6%, 18.8%, 19%, 19.2%, 19.4%, 19.6%, 19.8%, 20%, 20.2%, 20.4%, 20.6%, 20.8%, 21%, 21.2%, 21.4%, 21.6%, 21.8%, 220%, 22.20%, 22.40%, 22.6%, 22.8%, 23%, 23.2%, 23.4%, 23.6%, 23.8%, 24%, 24.2%, 24.4%, 24.6%, 24.8%, 25%, 25.2%, 25.4%, 25.6%, 25.8%, 26%, 26.2%, 26.4%, 26.6%, 26.8%, 27%, 27.2%, 27.4%, 27.6%, 27.8%, 28%, 28.2%, 28.4%, 28.6%, 28.8%, 29%, 29.2%, 29.4%, 29.6%, 29.8%, 30%, 30.2%, 30.4%, 30.6%, 30.8%, 31%, 31.2%, 31.4%, 31.6%, 31.8%, 32%, 32.2%, 32.4%, 32.6%, 32.8%, 33%, 33.2%, 33.4%, 33.6%, 33.8%, 34%, 34.2%, 34.4%, 34.6%, 34.8%, 35%, 35.2%, 35.4%, 35.6%, 35.8%, 36%, 36.2%, 36.4%, 36.6%, 36.8%, 37%, 37.2%, 37.4%, 37.6%, 37.8%, 38%, 38.2%, 38.4%, 38.6%, 38.8%, 39%, 39.2%, 39.4%, 39.6%, 39.8%, 400%, 40.20%, 40.40%, 40.6%, 40.80%, 41%, 41.2%, 41.4%, 41.6%, 41.8%, 42%, 42.2%, 42.4%, 42.6%, 42.8%, 43%, 43.2%, 43.4%, 43.6%, 43.8%, 44%, 44.2%, 44.4%, 44.6%, 44.8%, 45%, 45.2%, 45.4%, 45.6%, 45.8%, 46%, 46.2%, 46.4%, 46.6%, 46.8%, 47%, 47.2%, 47.4%, 47.6%, 47.8%, 48%, 48.2%, 48.4%, 48.6%, 48.8%, 49%, 49.2%, 49.4%, 49.6%, 49.8%, or up to about 50% as represented by (wt/wt) percent over total fatty acids.

In some embodiments, a composition comprises added short chain fatty acids. Short chain fatty acids can be added in the form of a plant oil. Plant oils are described elsewhere herein but in some embodiments a plant oil is selected from the group consisting of soybean oil, palm oil, coconut oil, almond oil, avocado oil, cocoa butter oil, corn oil, cottonseed oil, flax seed oil, grapeseed oil, hemp oil, olive oil, palm kernel oil, peanut oil, pumpkin seed oil, rice bran oil, safflower seed oil, sesame seed oil, sunflower seed oil, walnut oil, and combinations thereof.

### Expression of Transgenes in Plant Cells

Provided are compositions and methods of expressing a transgene in a plant cell, the method comprising: (i) providing a plant cell lacking at least one endogenous promoter; (ii) transforming the plant cell with a nucleic acid comprising a promoter and a transgene, wherein the promoter has the same sequence as the endogenous promoter; and (ii) maintaining the plant cell under conditions wherein the transgene is expressed. In some embodiments, the plant cell is a dicot cell from soybean, lima bean, Arabidopsis, tobacco, rice, maize, barley, sorghum, wheat or oat. In some embodiments, the plant cell is a monocot cell from turf grass, maize, rice, oat, wheat, barley, sorghum, orchid, iris, lily, onion, palm, or duckweed. In some embodiments, the promoter is the Glycinin 1 or Seed 2 promoter. In some embodiments, the transgene is a mammalian, avian, or plant gene. In some embodiments, the transgene encodes a milk protein selected from α-S1 casein, α-S2 casein, β-casein, κ-casein, para-κ-casein, 0-lactoglobulin, α-lactalbumin, lysozyme, lactoferrin, lactoperoxidase, serum albumin, or an immunoglobulin. In some embodiments, a transgene encodes an avian protein selected from ovalbumin, ovotransferrin, ovoglobulin, and lysozyme. In some embodiments, a transgene encodes a plant protein selected from oleosins, leghemoglobin, extension-like protein family, prolamin, glutenin, gamma-kafirin preprotein, α-globulin, basic 7S globulin precursor, 2S albumin, β-conglycinins, glycinins, canein, zein, patatin, kunitz-trypsin inhibitor, bowman-birk inhibitor, and cystatine.

Provided is a plant cell lacking at least one endogenous promoter; wherein the plant cell comprises a nucleic acid comprising a promoter and a transgene; wherein the promoter has the same sequence as the endogenous promoter.

Provided in Tables 23-31 are exemplary plants and varieties of the same that can be utilized in any of the compositions and methods of the disclosure.

TABLE 23
Exemplary Conventional Maturity Group IV Varieties

| | |
|---|---|
| Variety of Interest | Same Varieties as the Variety of Interest |

| | | |
|---|---|---|
| Armor 49-C3 | Progeny P5191 | GoSoy Leland |
| GoSoy Ireane | USG Ellis | |
| GoSoy Leland | Armor 49-C3 | Progeny P5191 |
| Progeny P5191 | GoSoy Leland | Armor 49-C3 |
| USG Ellis | GoSoy Ireane | |

TABLE 24
Exemplary Maturity Group IV Varieties (Enlist)

| Variety of Interest | Same Varieties as the Variety of Interest | |
|---|---|---|
| Bravo B4609E | Local Seed ZS4694E3S | |
| Dyna-Gro S42EN89 | Progeny P4241 E3 | |
| Dyna-Gro S46EN29 | Terral REV 4990E | Pioneer P49T62E |
| GoSoy 482E18 | USG 7479ET | |
| Local Seed ZS4694E3S | Bravo B4609E | |
| Pioneer P48T22E | Terral REV 5190E | Progeny P5211 E3 |
| Pioneer P49T62E | Dyna-Gro S46EN29 | Terral REV 4990E |
| Progeny P4241 E3 | Dyna-Gro S42EN89 | |
| Progeny P5211 E3 | Pioneer P48T22E | Terral REV 5190E |
| Terral REV 4990E | Pioneer P49T62E | Dyna-Gro S46EN29 |
| Terral REV 5190E | Progeny P5211 E3 | Pioneer P48T22E |
| USG 7479ET | GoSoy 482E18 | |

TABLE 25
Exemplary Maturity Group IV Varieties (LLGT27)

| Variety of Interest | Same Varieties as the Variety of Interest |
|---|---|
| Armor 44-Z28 | Stine 44GA00 |
| Stine 44GA00 | Armor 44-Z28 |

TABLE 26
Exemplary Maturity Group IV Varieties (Liberty Link)

| Variety of Interest | Same Varieties as the Variety of Interest | | | | | | |
|---|---|---|---|---|---|---|---|
| Armor 44-L21 | Cerdenz CZ | Delta Grow | Dyna-Gro | Croplan | NK S44-C5L | | |
| | 4548LL | DG4587LL | S44LS76 | LC4467S | | | |
| Armor 47-L10 | Stine 47LF32 | Pioneer | Croplan | Delta Grow | Pioneer 45L57 | NK S47-F6L | Cerdenz |
| | | P45T39L | LC4814 | DG4781LL | | | CZ4748LL |
| Armor 47-Z01 | Progeny | Cerdenz CZ | | | | | |
| | P4565 LR | 4539GTLL | | | | | |
| Armor 495L | Dyna-Gro | Progeny | | | | | |
| | S48LL23 | P4819 LL | | | | | |
| Cerdenz CZ | Armor 47-Z01 | Progeny | | | | | |
| 4539GTLL | | P4565 LR | | | | | |
| Cerdenz CZ | Delta Grow | Dyna-Gro | Croplan | NK S44-C5L | Armor 44-L21 | | |
| 4548LL | DG4587LL | S44LS76 | LC4467S | | | | |
| Cerdenz | Stine 40LF23 | Stine 40LF23 | | | | | |
| CZ4044LL | | | | | | | |
| Cerdenz | Dyna-Gro | | | | | | |
| CZ4222LL | S45LL97 | | | | | | |
| Cerdenz | Armor 47-L10 | Stine 47LF32 | Pioneer | Croplan | Delta Grow | Pioneer | NK S47-F6L |
| CZ4748LL | | | P45T39L | LC4814 | DG4781LL | 45L57 | |
| Cerdenz | Stine 48LI32 | Croplan | | | | | |
| CZ4820 LL | | LC4850 | | | | | |
| Cerdenz | GoSoy | Stine 51LI32 | | | | | |
| CZ4938 LL | 49L17LL | | | | | | |
| Credenz | Stine 49LH02 | | | | | | |
| CZ4918 LL | | | | | | | |
| Croplan | NK S44-C5L | Armor | Cerdenz CZ | Delta Grow | Dyna-Gro | | |
| LC4467S | | 44-L21 | 4548LL | DG4587LL | S44LS76 | | |
| Croplan | Delta Grow | Pioneer 45L57 | NK S47-F6L | Cerdenz | Armor 47-L10 | Stine | Pioneer |
| LC4814 | DG4781LL | | | CZ4748LL | | 47LF32 | P45T39L |
| Croplan | Cerdenz | Stine 48LI32 | | | | | |
| LC4850 | CZ4820 LL | | | | | | |
| Croplan | Progeny | | | | | | |
| LST4885S | P4814LLS | | | | | | |
| Delta Grow | Dyna-Gro | Croplan | NK S44-C5L | Armor 44-L21 | Cerdenz CZ | | |
| DG4587LL | S44LS76 | LC4467S | | | 4548LL | | |
| Delta Grow | Pioneer 45L57 | NK S47-F6L | Cerdenz | Armor 47-L10 | Stine | Pioneer | Croplan |
| DG4781LL | | | CZ4748LL | | 47LF32 | P45T39L | LC4814 |
| Delta Grow | NK S49-U6LS | Great Heart | Dyna-Gro | Stine 50LF32 | | | |
| DG4977LL/STS | | GT-501CLS | S49LS65 | | | | |
| Dyna-Gro | Croplan | NK S44-C5L | Armor 44-L21 | Cerdenz CZ | Delta Grow | | |
| S44LS76 | LC4467S | | | 4548LL | DG4587LL | | |
| Dyna-Gro | Cerdenz | | | | | | |
| S45LL97 | CZ4222LL | | | | | | |
| Dyna-Gro | Progeny | Armor 495L | | | | | |
| S48LL23 | P4819 LL | | | | | | |
| Dyna-Gro | GoSoy 4913LL | HBK LL4953 | Pioneer | Progeny | Stine | | |
| S49LL34 | | | P53T62L | P4930LL | 50LD02 | | |
| Dyna-Gro | Stine 50LF32 | Delta Grow | NK S49-U6LS | Great Heart | | | |
| S49LS65 | | DG4977LL/STS | | GT-501CLS | | | |
| GoSoy 4913LL | HBK LL4953 | Pioneer | Progeny | Stine 50LD02 | Dyna-Gro | | |
| | | P53T62L | P4930LL | | S49LL34 | | |
| GoSoy 49L17LL | Stine 51LI32 | Cerdenz | | | | | |
| | | CZ4938 LL | | | | | |
| Great Heart | Dyna-Gro | Stine | Delta Grow | NK S49-U6LS | | | |
| GT-501CLS | S49LS65 | 50LF32 | DG4977LL/STS | | | | |
| HBK LL4953 | Pioneer | Progeny | Stine 50LD02 | Dyna-Gro | GoSoy | | |
| | P53T62L | P4930LL | | S49LL34 | 4913LL | | |
| NK S44-C5L | Armor 44-L21 | Cerdenz CZ | Delta Grow | Dyna-Gro | Croplan | | |
| | | 4548LL | DG4587LL | S44LS76 | LC4467S | | |
| NK S47-F6L | Cerdenz | Armor 47-L10 | Stine 47LF32 | Pioneer | Croplan | Delta Grow | Pioneer |
| | CZ4748LL | | | P45T39L | LC4814 | DG4781LL | 45L57 |
| NK S49-U6LS | Great Heart | Dyna-Gro | Stine 50LF32 | Delta Grow | | | |
| | GT-501CLS | S49LS65 | | DG4977LL/STS | | | |
| Pioneer 45L57 | NK S47-F6L | Cerdenz | Armor 47-L10 | Stine 47LF32 | Pioneer | Croplan | Delta Grow |
| | | CZ4748LL | | | P45T39L | LC4814 | DG4781LL |
| Pioneer P45A29L | Terral REV | | | | | | |
| | 46L99 | | | | | | |
| Pioneer P45T39L | Croplan | Delta Grow | Pioneer 45L57 | NK S47-F6L | Cerdenz | Armor 47-L10 | Stine 47LF32 |
| | LC4814 | DG4781LL | | | CZ4748LL | | |
| Pioneer P47A76L | Terral REV | | | | | | |
| | 47L38 | | | | | | |
| Pioneer P49T31L | Terral REV | | | | | | |
| | 48L63 | | | | | | |
| Pioneer P50A78L | Terral REV | | | | | | |
| | 49L88 | | | | | | |
| Pioneer P53T62L | Progeny | Stine | Dyna-Gro | GoSoy | HBK LL4953 | | |
| | P4930LL | 50LD02 | S49LL34 | 4913LL | | | |
| Progeny | Cerdenz CZ | Armor | | | | | |
| P4565 LR | 4539GTLL | 47-Z01 | | | | | |
| Progeny | Croplan | | | | | | |
| P4814LLS | LST4885S | | | | | | |
| Progeny | Armor 495L | Dyna-Gro | | | | | |
| P4819 LL | | S48LL23 | | | | | |
| Progeny | Stine 50LD02 | Dyna-Gro | GoSoy | HBK LL4953 | Pioneer | | |
| P4930LL | | S49LL34 | 4913LL | | P53T62L | | |
| Stine 40LF23 | Cerdenz | | | | | | |
| | CZ4044LL | | | | | | |
| Stine 47LF32 | Pioneer | Croplan | Delta Grow | Pioneer | NK S47-F6L | Cerdenz | Armor 47-L10 |
| | P45T39L | LC4814 | DG4781LL | 45L57 | | CZ4748LL | |
| Stine 48LI32 | Croplan | Cerdenz | | | | | |
| | LC4850 | CZ4820 LL | | | | | |
| Stine 49LH02 | Credenz | | | | | | |
| | CZ4918 LL | | | | | | |
| Stine 50LD02 | Dyna-Gro | GoSoy | HBK LL4953 | Pioneer | Progeny | | |
| | S49LL34 | 4913LL | | P53T62L | P4930LL | | |
| Stine 50LF32 | Delta Grow | NK S49-U6LS | Great Heart | Dyna-Gro | | | |
| | DG4977LL/STS | | GT-501CLS | S49LS65 | | | |
| Stine 51LI32 | Cerdenz | GoSoy | | | | | |
| | CZ4938 LL | 49L17LL | | | | | |
| Terral REV 46L99 | Pioneer P45A29L | | | | | | |
| Terral REV 47L38 | Pioneer P47A76L | | | | | | |
| Terral REV 48L63 | Pioneer P49T31L | | | | | | |
| Terral REV 49L88 | Pioneer P50A78L | | | | | | |

TABLE 27
Exemplary Maturity Group IV Varieties (RR1/RR2Y)

| Variety of Interest | Same Varieties as the Variety of Interest | | |
|---|---|---|---|
| AgVenture 48E3RR | Terral REV 49R94 | | |
| Armor 44-R08 | Progeny P4211RY | Dyna-Gro 39RY43 | Croplan R2C4391 |
| Armor 47-R70 | Progeny P4757RY | Delta Grow | Croplan R2C4775 |
| | | DG4790GENRR2Y | |
| Armor 49-R44 | Croplan R2C4914S | | |
| Croplan R2C4391 | Armor 44-R08 | Progeny P4211RY | Dyna-Gro 39RY43 |
| Croplan R2C4775 | Armor 47-R70 | Progeny P4757RY | Delta Grow |
| | | | DG4790GENRR2Y |
| Croplan R2C4914S | Armor 49-R44 | | |
| Delta Grow | Croplan R2C4775 | Armor 47-R70 | Progeny P4757RY |
| DG4790GENRR2Y | | | |
| Dyna-Gro 39RY43 | Croplan R2C4391 | Armor 44-R08 | Progeny P4211RY |
| Dyna-Gro S43RY95 | Mycogen 5N433R2 | | |
| Dyna-Gro S49RY25 | Great Heart GT516CR2 | | |
| Great Heart GT516CR2 | Dyna-Gro S49RY25 | | |
| Mycogen 5N433R2 | Dyna-Gro S43RY95 | | |
| Progeny P4211RY | Dyna-Gro 39RY43 | Croplan R2C4391 | Armor 44-R08 |
| Progeny P4757RY | Delta Grow | Croplan R2C4775 | Armor 47-R70 |
| | DG4790GENRR2Y | | |
| Terral REV 49R94 | AgVenture 48E3RR | | |
| AgVenture 48E3RR | Terral REV 49R94 | | |
| Armor 44-R08 | Progeny P4211RY | Dyna-Gro 39RY43 | Croplan R2C4391 |
| Armor 47-R70 | Progeny P4757RY | Delta Grow | Croplan R2C4775 |
| | | DG4790GENRR2Y | |
| Armor 49-R44 | Croplan R2C4914S | | |
| Croplan R2C4391 | Armor 44-R08 | Progeny P4211RY | Dyna-Gro 39RY43 |
| Croplan R2C4775 | Armor 47-R70 | Progeny P4757RY | Delta Grow |
| | | | DG4790GENRR2Y |
| Croplan R2C4914S | Armor 49-R44 | | |
| Delta Grow | Croplan R2C4775 | Armor 47-R70 | Progeny P4757RY |
| DG4790GENRR2Y | | | |
| Dyna-Gro 39RY43 | Croplan R2C4391 | Armor 44-R08 | Progeny P4211RY |
| Dyna-Gro S43RY95 | Mycogen 5N433R2 | | |
| Dyna-Gro S49RY25 | Great Heart GT516CR2 | | |
| Great Heart GT516CR2 | Dyna-Gro S49RY25 | | |
| Mycogen 5N433R2 | Dyna-Gro S43RY95 | | |
| Progeny P4211RY | Dyna-Gro 39RY43 | Croplan R2C4391 | Armor 44-R08 |
| Progeny P4757RY | Delta Grow | Croplan R2C4775 | Armor 47-R70 |
| | DG4790GENRR2Y | | |
| Terral REV 49R94 | AgVenture 48E3RR | | |

TABLE 28
Exemplary Maturity Group IV Varieties (Xtend)

| Variety of Interest | Same Varieties as the Variety of Interest | | | | | | |
|---|---|---|---|---|---|---|---|
| AgVenture 47W2X | Terral REV 4857X | | | | | | |
| Armor 42-D27 | Dyna-Gro S41XS98 | | | | | | |
| Armor 44-D40 | Dyna-Gro S44XS57 | Local Seed | | | | | |
| | | LS4458XS | | | | | |
| Armor 46-D08 | Croplan RX4516S | Progeny | Local | Dyna-Gro | MorSoy 4616 | | |
| | | P4620RXS | LS4565XS | S45XS37 | RXT | | |
| Armor 47-D17 | Progeny P4516RXS | Dyna-Gro | Croplan | | | | |
| | | S45XS66 | RX4555S | | | | |
| Armor 47-D93 | Mission Seed | | | | | | |
| | A4657NSXR2 | | | | | | |
| Armor 48-D02 | Mission Seed A4828X | | | | | | |
| Armor 48-D24 | Hefty H48X7 | Local Seed | MorSoy 4846 | Progeny | Dyna-Gro | Delta Grow | Croplan |
| | | LS4966X | RXT | P4816RX | S48XT56 | DG48X45 | RX4825 |
| Armor 48-D87 | Croplan RX4927 | | | | | | |
| Armor 49-D13 | Mission Seed A4950X | | | | | | |
| Armor 49-D14 | Dyna-Gro S48XT90 | | | | | | |
| Cerdenz CZ4869X | Local Seed LS4677X | | | | | | |
| Credenz CZ 4730X | Dyna-Gro S47XT20 | | | | | | |
| Croplan RX4516S | Progeny P4620RXS | Local LS4565XS | Dyna-Gro | MorSoy 4616 | Armor 46-D08 | | |
| | | | S45XS37 | RXT | | | |
| Croplan RX4555S | Armor 47-D17 | Progeny | Dyna-Gro | | | | |
| | | P4516RXS | S45XS66 | | | | |
| Croplan RX4817S | Dyna-Gro S48XS78 | Mission Seed | | | | | |
| | | A4847NSXR2 | | | | | |
| Croplan RX4825 | Armor 48-D24 | Hefty H48X7 | Local Seed | MorSoy 4846 | Progeny | Dyna-Gro | Delta Grow |
| | | | LS4966X | RXT | P4816RX | S48XT56 | DG48X45 |
| Croplan RX4926 | Dyna-Gro S49XT07 | | | | | | |
| Croplan RX4927 | Armor 48-D87 | | | | | | |
| Delta Grow | Croplan RX4825 | Armor 48-D24 | Hefty H48X7 | Local Seed | MorSoy 4846 | Progeny | Dyna-Gro |
| DG48X45 | | | | LS4966X | RXT | P4816RX | S48XT56 |
| Dyna-Gro S41XS98 | Armor 42-D27 | | | | | | |
| Dyna-Gro S43XS27 | Mission Seed | | | | | | |
| | A4637NSXR2 | | | | | | |
| Dyna-Gro S44XS57 | Armor 44-D40 | Local Seed | | | | | |
| | | LS4458XS | | | | | |
| Dyna-Gro S45XS37 | MorSoy 4616 RXT | Armor 46-D08 | Croplan | Progeny | Local | | |
| | | | RX4516S | P4620RXS | LS4565XS | | |
| Dyna-Gro S45XS66 | Croplan RX4555S | Armor 47-D17 | Progeny | | | | |
| | | | P4516RXS | | | | |
| Dyna-Gro S46XS60 | Local Seed LS4795XS | | | | | | |
| Dyna-Gro S47XT20 | Credenz CZ 4730X | | | | | | |
| Dyna-Gro S48XS78 | Mission Seed | Croplan RX4817S | | | | | |
| | A4847NSXR2 | | | | | | |
| Dyna-Gro S48XT56 | Delta Grow DG48X45 | Croplan RX4825 | Armor 48-D24 | Hefty H48X7 | Local Seed | MorSoy 4846 | Progeny |
| | | | | | LS4966X | RXT | P4816RX |
| Dyna-Gro S48XT90 | Armor 49-D14 | | | | | | |
| Dyna-Gro S49XS76 | Hefty H49X7S | Progeny | USG | | | | |
| | | P5016RXS | 7496XTS | | | | |
| Dyna-Gro S49XT07 | Croplan RX4926 | | | | | | |
| Hefty H48X7 | Local Seed LS4966X | MorSoy 4846 | Progeny | Dyna-Gro | Delta Grow | Croplan | Armor 48- |
| | | RXT | P4816RX | S48XT56 | DG48X45 | RX4825 | D24 |
| Hefty H49X7S | Progeny P5016RXS | USG 7496XTS | Dyna-Gro | | | | |
| | | | S49XS76 | | | | |
| Local LS4565XS | Dyna-Gro S45XS37 | MorSoy 4616 | Armor 46-D08 | Croplan | Progeny | | |
| | | RXT | | RX4516S | P4620RXS | | |
| Local Seed | Armor 44-D40 | Dyna-Gro | | | | | |
| LS4458XS | | S44XS57 | | | | | |
| Local Seed | Cerdenz CZ4869X | | | | | | |
| LS4677X | | | | | | | |
| Local Seed | Dyna-Gro S46XS60 | | | | | | |
| LS4795XS | | | | | | | |
| Local Seed | MorSoy 4846 RXT | Progeny P4816RX | Dyna-Gro | Delta Grow | Croplan | Armor 48-D24 | Hefty H48X7 |
| LS4966X | | | S48XT56 | DG48X45 | RX4825 | | |
| Mission Seed | MorSoy 4447 RXT | Progeny | | | | | |
| A4448X | | P4444RXS | | | | | |
| Mission Seed | Progeny P4570RXS | | | | | | |
| A4618X | | | | | | | |
| Mission Seed | Dyna-Gro S43XS27 | | | | | | |
| A4637NSXR2 | | | | | | | |
| Mission Seed | Armor 47-D93 | | | | | | |
| A4657NSXR2 | | | | | | | |
| Mission Seed | Armor 48-D02 | | | | | | |
| A4828X | | | | | | | |
| Mission Seed | Croplan RX4817S | Dyna-Gro | | | | | |
| A4847NSXR2 | | S48XS78 | | | | | |
| Mission Seed | Armor 49-D13 | | | | | | |
| A4950X | | | | | | | |
| MorSoy 4447 RXT | Progeny P4444RXS | Mission Seed | | | | | |
| | | A4448X | | | | | |
| MorSoy 4616 RXT | Armor 46-D08 | Croplan RX4516S | Progeny | Local | Dyna-Gro | | |
| | | | P4620RXS | LS4565XS | S45XS37 | | |
| MorSoy 4846 RXT | Progeny P4816RX | Dyna-Gro | Delta Grow | Croplan | Armor 48-D24 | Hefty H48X7 | Local Seed |
| | | S48XT56 | DG48X45 | RX4825 | | | LS4966X |
| Progeny P4444RXS | Mission Seed A4448X | MorSoy 4447 | | | | | |
| | | RXT | | | | | |
| Progeny P4516RXS | Dyna-Gro S45XS66 | Croplan RX4555S | Armor 47-D17 | | | | |
| Progeny P4570RXS | Mission Seed A4618X | | | | | | |
| Progeny P4620RXS | Local LS4565XS | Dyna-Gro | MorSoy 4616 | Armor 46-D08 | Croplan | | |
| | | S45XS37 | RXT | | RX4516S | | |
| Progeny P4816RX | Dyna-Gro S48XT56 | Delta Grow | Croplan | Armor 48-D24 | Hefty H48X7 | Local Seed | MorSoy 4846 |
| | | DG48X45 | RX4825 | | | LS4966X | RXT |
| Progeny P5016RXS | USG 7496XTS | Dyna-Gro | Hefty H49X7S | | | | |
| | | S49XS76 | | | | | |
| Terral REV 4857X | AgVenture 47W2X | | | | | | |
| USG 7496XTS | Dyna-Gro S49XS76 | Hefty H49X7S | Progeny | | | | |
| | | | P5016RXS | | | | |

TABLE 29
Exemplary Maturity Group V Varieties (Liberty Link)

Variety of

| Interest | Same Varieties as the Variety of Interest | | | |
|---|---|---|---|---|
| Armor 501L | GoSoy 4912 LL | Dyna-Gro | Delta Grow | Pioneer P95L01 |
| | | S50LL25 | DG4967LL | |
| Cerdenz | Croplan LCS215 | Dyna-Gro | Stine 51LE20 | Delta Grow |
| CZ5242LL | | S52LL66 | | DG5067LL |
| Croplan | Dyna-Gro | GoSoy 5515 LL | Progeny | |
| L5T5555S | S55LS75 | | P5414LLS | |
| Croplan LCS215 | Dyna-Gro | Stine 51LE20 | Delta Grow | Cerdenz |
| | S52LL66 | | DG5067LL | CZ5242LL |
| Delta Grow | Pioneer P95L01 | Armor 501L | GoSoy 4912 LL | Dyna-Gro |
| DG4967LL | | | | S50LL25 |
| Delta Grow | Cerdenz | Croplan LCS215 | Dyna-Gro | Stine 51LE20 |
| DG5067LL | CZ5242LL | | S52LL66 | |
| Dyna-Gro | Delta Grow | Pioneer P95L01 | Armor 501L | GoSoy 4912 LL |
| S50LL25 | DG4967LL | | | |
| Dyna-Gro | Stine 51LE20 | Delta Grow | Cerdenz | Croplan LCS215 |
| S52LL66 | | DG5067LL | CZ5242LL | |
| Dyna-Gro | GoSoy 5515 LL | Progeny | Croplan | |
| S55LS75 | | P5414LLS | L5T5555S | |
| GoSoy 4912 LL | Dyna-Gro | Delta Grow | Pioneer P95L01 | Armor 501L |
| | S50LL25 | DG4967LL | | |
| GoSoy 5515 LL | Progeny | Croplan | Dyna-Gro | |
| | P5414LLS | L5T5555S | S55LS75 | |
| Pioneer P52A43L | Terral REV | | | |
| | 54L18 | | | |
| Pioneer P95L01 | Armor 501L | GoSoy 4912 LL | Dyna-Gro | Delta Grow |
| | | | S50LL25 | DG4967LL |
| Progeny | Croplan | Dyna-Gro | GoSoy 5515 LL | |
| P5414LLS | L5T5555S | S55LS75 | | |
| Stine 51LE20 | Delta Grow | Cerdenz | Croplan LCS215 | Dyna-Gro |
| | DG5067LL | CZ5242LL | | S52LL66 |
| Terral REV | Pioneer P52A43L | | | |
| 54L18 | | | | |

TABLE 30
Exemplary Maturity Group V Varieties (RR1/RR2Y)

| Variety of Interest | Same Varieties as the Variety of Interest | | |
|---|---|---|---|
| Ag Venture 48E3RR | Terral REV 49R94 | | |
| Armor 55-R68 | Delta Grow | | |
| | DG5580GENRR2Y | | |
| Armor 55-R68 | Delta Grow DG5580 GEN | | |
| | RR2Y | | |
| Armor 57-R17 | Dyna-Gro S56RY84 | Progeny | Croplan |
| | | P5555RY | R2C5673 |
| Croplan R2C5225S | Progeny P5226RYS | | |
| Croplan R2C5265 | Dyna-Gro S52RY75 | | |
| Croplan R2C5656 | Progeny P5752RY | | |
| Croplan R2C5673 | Armor 57-R17 | Dyna-Gro | Progeny |
| | | S56RY84 | P5555RY |
| Delta Grow DG5580 GEN | Armor 55-R68 | | |
| RR2Y | | | |
| Delta Grow | Armor 55-R68 | | |
| DG5580GENRR2Y | | | |
| Dyna-Gro S52RY75 | Croplan R2C5265 | | |
| Dyna-Gro S56RY84 | Progeny P5555RY | Croplan | Armor 57-R17 |
| | | R2C5673 | |
| Progeny P5226RYS | Croplan R2C5225S | | |
| Progeny P5555RY | Croplan R2C5673 | Armor 57-R17 | Dyna-Gro |
| | | | S56RY84 |
| Progeny P5752RY | Croplan R2C5656 | | |
| Terral REV 49R94 | AgVenture 48E3RR | | |

TABLE 31
Exemplary Maturity Group V Varieties (Xtend)

| Variety of Interest | Same Varieties as the Variety of Interest |
|---|---|
| Croplan RX5667 | Progeny P5688RX |
| Progeny P5688RX | Croplan RX5667 |

As can be seen from the above tables, the soybean varieties that are applicable for the genomic architecture edits of the present disclosure may include both conventional and genetically modified varieties. A multitude of genetic events can be utilized in the host soybean genomic background (e.g. the events contained in the GMO soybean of the aforementioned tables), in combination with the genetic edits taught herein associated with the recombinant production of proteins and/or any of the various constructs taught herein. Further, although MG 4 and MG 5 varieties have been illustrated above, the disclosure is applicable to all soybean maturity groups.

### EXAMPLES

The following examples are given for the purpose of illustrating various embodiments of the disclosure and are not meant to limit the present disclosure in any fashion. Changes therein and other uses which are encompassed within the spirit of the disclosure, as defined by the scope of the claims, will be recognized by those skilled in the art

The following experiments demonstrate different recombinant fusion constructs comprising a milk protein (e.g., a casein) and at least one other protein, as well as methods of producing and testing the fusion proteins. While the examples below describe expression in soybean, it will be understood by those skilled in the art that the constructs and methods disclosed herein may be tailored for expression in any organism.

The following examples also demonstrate the production of various cheese compositions and characterization of their properties. Traditionally cheese is made from milk, which comprises a mixture of casein proteins. To test whether a cheese composition having acceptable organoleptic and physical properties could be made using only one casein protein, or different combinations/ratios of casein proteins as compared to that found in any mammalian milk, various experiments described below were performed. While the examples below utilize isolated caseins isolated from bovine milk, it will be understood by those skilled in the art that the recipes and methods disclosed herein may be tailored for use with other isolated caseins and recombinant caseins, including caseins expressed in a plant.

### Example 1: Construction of Expression Vectors for Plant Transformation for Stable Expression of Recombinant Fusion Proteins

### Binary Vector Design

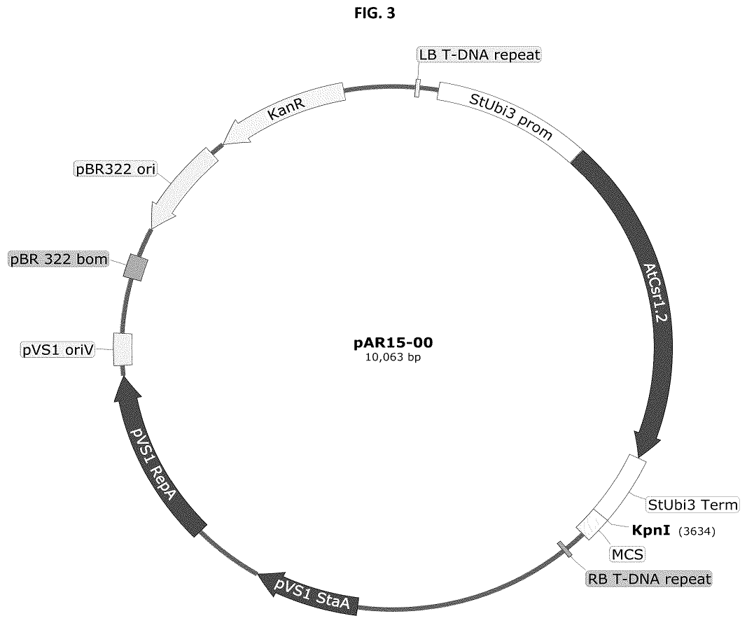

While a number of vectors may be utilized for expression of the fusion proteins disclosed herein, the example constructs described below were built in the binary pCAMBIA3300 (Creative Biogene, VET1372) vector, which was customized for soybean transformation and selection. In order to modify the vector, pCAMBIA3300 was digested with HindIII and AseI allowing the release of the vector backbone (LB T-DNA repeat_KanR_pBR322 oripBR322 bom_pVS1 oriV_pVsl repA_pVS1 StaA_RB T-DNA repeat). The 6598 bp vector backbone was gel extracted and a synthesized multiple cloning site (MCS) was ligated via In-Fusion cloning (In-Fusion® HD Cloning System CE, available on the world wide web at clontech.com) to allow modular vector modifications. A cassette containing the Arabidopsis thaliana Csr1.2 gene for acetolactate synthase was added to the vector backbone to be used as a marker for herbicide selection of transgenic plants. In order to build this cassette, the regulatory sequences from Solanum tuberosum ubiquitin/ribosomal fusion protein promoter (StUbi3 prom; −1 to −922 bp) and terminator (StUbi3 term; 414 bp) (GenBank accession no. L22576.1) were fused to the mutant (S653N) acetolactate synthase gene (Csr1.2; GenBank accession no. X51514.1) (Sathasivan et al, 1990; Ding et al, 2006) to generate imazapyr-resistant traits in soybean plants. The selectable marker cassette was introduced into the digested (EcoRI) modified vector backbone via In-Fusion cloning to form vector pAR15-00 (FIG. 3).

Recombinant DNA constructs were designed to express milk proteins in transgenic plants. The coding regions of the expression cassettes outlined below contain a fusion of codon-optimized nucleic acid sequences encoding bovine milk proteins, or a functional fragment thereof. To enhance protein expression in soybean, the nucleic acid sequences encoding β-lactoglobulin (GenBank accession no. X14712.1), κ-casein (GenBank accession no. CAA25231), β-casein (GenBank accession no. M15132.1), and aS1-casein (GenBank accession no. X59836.1) were codon optimized using Glycine max codon bias and synthesized (available on the world wide web at idtdna.com/CodonOpt). The signal sequences were removed (i.e., making the constructs “truncated”) and the new versions of the genes were renamed as OLG1 (0-lactoglobulin version 1, SEQ ID NO: 9), OLG2 (0-lactoglobulin version 2, SEQ ID NO: 11), OLG3 (0-lactoglobulin version 3, SEQ ID NO: 12), OLG4 (0-lactoglobulin version 4, SEQ ID NO: 13), OKC1-T (Optimized κ-casein Truncated version 1, SEQ ID NO: 3), paraOKC1-T (only the para-κportion of OKC1-T, SEQ ID NO: 1), OBC-T2 (Optimized β-casein Truncated version 2, SEQ ID NO: 5), and OaS1-T (Optimized aS1-casein Truncated version 1, SEQ ID NO: 7). As will be understood by those skilled in the art, codon optimized nucleic acid sequences can present from about 60% to about 100% identity to the native version of the nucleic acid sequence.

All the expression cassettes described below and shown in FIG. 4-FIG. 9 contained codon-optimized nucleic acid sequences encoding bovine milk proteins, or a functional fragment thereof, a seed specific promoter, a 5′UTR, a signal sequence (Sig) that directs foreign proteins to the protein storage vacuoles, and a termination sequence. In some versions of the constructs a linker such as a linker comprising a chymosin cleavage site (FM), was placed between the two proteins and/or a C-terminal KDEL sequence for ER retention was included. Expression cassettes were inserted in the pAR15-00 vector described above utilizing a KpnI restriction site with the MCS (FIG. 3). Coding regions and regulatory sequences are indicated as blocks (not to scale) in FIG. 4-FIG. 9.

κ-casein-β-lactoglobulin fusion with KDEL

Shown in FIG. 4 is an example expression cassette comprising κ-casein (OKC1-T, SEQ ID NO: 3) and β-lactoglobulin (OLG1, SEQ ID NO: 9). The regulatory sequences that were used in order to produce the heterologous milk proteins in soybean seeds include the promoter of the beta-phaseolin storage protein gene (PvPhas prom; −1 to −1543; GenBank accession no. J01263.1, SEQ ID NO: 18); the 5′UTR of the arc5-1 gene (arc5′UTR; −1 to −13; GenBank accession no. Z50202, SEQ ID NO: 20) (De Jaeger et al, 2002); the signal peptide of Lectin 1 gene 1 (sig10; +1 to +93; GenBank accession no. Glyma.02G012600, SEQ ID NO: 14) (Darnowski et al, 20020); and, the 3′UTR of the arc5-1 gene, (arc term 1197 bp; GenBank accession no. Z50202.1, SEQ ID NO: 21)(De Jaeger et al, 2002). A C-terminal KDEL (SEQ ID NO: 23) was also included for ER retention.

β-casein-β-lactoglobulin fusion with linker

Shown in FIG. 5 is an example expression cassette comprising β-casein (OBC-T2, SEQ ID NO: 5) and β-lactoglobulin (OLG1, SEQ ID NO: 9). The regulatory sequences that were used in order to produce the heterologous milk proteins in soybean seeds include the promoter of the beta-phaseolin storage protein gene (PvPhas prom; −1 to −1543; GenBank accession no. J01263.1, SEQ ID NO: 18); the 5′UTR of the arc5-1 gene (arc5′UTR; −1 to −13; GenBank accession no. Z50202, SEQ ID NO: 20) (De Jaeger et al, 2002); the signal peptide of Lectin 1 gene 1 (sig10; +1 to +93; accession no. Glyma.02G012600, SEQ ID NO: 14) (Darnowski et al, 2002); and, the 3′UTR of the arc5-1 gene, (arc term 1197 bp; accession no. Z50202.1, SEQ ID NO: 21) (De Jaeger, et al 2002). A linker comprising a chymosin cleavage site (FM) was inserted between the two proteins.

αS1-casein-β-lactoglobulin fusion with linker

Shown in FIG. 6 is an example expression cassette comprising αS1-casein (OaS1-T, SEQ ID NO: 7) and β-lactoglobulin (OLG1, SEQ ID NO: 9). The regulatory sequences that were used in order to produce the heterologous milk proteins in soybean seeds include the promoter of the beta-phaseolin storage protein gene (PvPhas prom; −1 to −1543; GenBank accession no. J01263.1, SEQ ID NO: 18); the 5′UTR of the arc5-1 gene (arc5′UTR; −1 to −13; GenBank accession no. Z50202, SEQ ID NO: 20) (De Jaeger et al, 2002); the signal peptide of Lectin 1 gene 1 (sig10; +1 to +93; accession no. Glyma.02G012600, SEQ ID NO: 14) (Darnowski et al, 2002); and, the 3′UTR of the arc5-1 gene, (arc term 1197 bp; GenBank accession no. Z50202.1, SEQ ID NO: 21)(De Jaeger et al, 2002). A linker comprising a chymosin cleavage site (FM) was inserted between the two proteins.

Para-κ-casein-β-lactoglobulin fusion with linker and KDEL

Shown in FIG. 7 is an example expression cassette comprising para-κ-casein (paraOKC1-T, SEQ ID NO: 1) and β-lactoglobulin (OLG1, SEQ ID NO: 9). The regulatory sequences that were used in order to produce the heterologous milk proteins in soybean seeds include the promoter of the beta-phaseolin storage protein gene (PvPhas prom; −1 to −1543; GenBank accession no. J01263.1, SEQ ID NO: 18); the 5′UTR of the arc5-1 gene (arc5′UTR; −1 to −13; GenBank accession no. Z50202, SEQ ID NO: 20) (De Jaeger et al, 2002); the signal peptide of Lectin 1 gene 1 (sig10; +1 to +93; GenBank accession no. Glyma.02G012600, SEQ ID NO: 14) (Darnowski et al, 2002); and, the 3′UTR of the arc5-1 gene, (arc term 1197 bp; GenBank accession no. Z50202.1, SEQ ID NO: 21) (De Jaeger et al 2002). A linker comprising a chymosin cleavage site (FM) was inserted between the two proteins and a C-terminal KDEL (SEQ ID NO: 23) was also included for ER retention.

Para-κ-casein-β-lactoglobulin fusion with linker

Shown in FIG. 8 is an example expression cassette comprising para-κ-casein (paraOKC1-T, SEQ ID NO: 1) and β-lactoglobulin (OLG1, SEQ ID NO: 9). The regulatory sequences that were used in order to produce the heterologous milk proteins in soybean seeds include the promoter of the beta-phaseolin storage protein gene (PvPhas prom; −1 to −1543; GenBank accession no. J01263.1, SEQ ID NO: 18); the 5′UTR of the arc5-1 gene (arc5′UTR; −1 to −13; GenBank accession no. Z50202, SEQ ID NO: 20) (De Jaeger et al, 2002); the signal peptide of Lectin 1 gene 1 (sig10; +1 to +93; GenBank accession no. Glyma.02G012600, SEQ ID NO: 14) (Darnowski et al, 2002); and, the 3′UTR of the arc5-1 gene, (arc term 1197 bp; GenBank accession no. Z50202.1, SEQ ID NO: 21) (De Jaeger et al, 2002). A linker comprising a chymosin cleavage site (FM) was inserted between the two proteins.

Fusion protein with Seed2 Promoter, Sig2 and nopaline synthase terminator

Shown in FIG. 9 is an example expression cassette comprising κ-casein (OKC1-T, SEQ ID NO: 3) and β-lactoglobulin (OLG1, SEQ ID NO: 9). The regulatory sequences that were used in order to produce the heterologous milk proteins in soybean seeds include the promoter and signal peptide of glycinin 1 (GmSeed2 (SEQ ID NO: 19):sig2 (SEQ ID NO: 16)) followed by the ER retention signal (KDEL) and the Nopaline synthase termination sequence (nos term, SEQ ID NO: 22).

### Exemplary Protein Co-expression Vector

Binary pCAMBIA3300 vectors individually encoding for: (1) a prolamin (e.g., Canein or Zein); (2) a milk protein (e.g., Casein); or (3) both a prolamin and a milk protein are generated to co-express a milk protein and prolamin in plant cells (See FIG. 26A-26G, FIG. 27). Co-expression of a milk protein and prolamin will result in generation of a protein body in the plant cell capable of shielding the milk protein from degradation or capable of reducing toxicity, if any, associated with recombinant expression of the milk protein in the plant cell.

Exemplary DNA constructs encoding fusion proteins

Exemplary fusion protein constructs are shown in Table 32 and summarized below.

Construct 1: Beta-casein-FM-AlphaS1-casein-FM-AlphaS1-casein-FM-Beta-casein

ARDV-02 contains the binary pCAMBIA3300 base vector with features such as the right border repeat from nopaline C58 T-DNA, (RB-T-DNA repeat) the stability protein from Pseudomonas plasmid pVS1 (pVS1 StaA), the replication protein from Pseudomonas plasmid pVS1 (pVS1 repA), origin of replication for the Pseudomonas plasmid pVS1 (pVS1 oriV), the basis of mobility region from pBR322 (bom), high-copy-number ColEl/pMB1/pBR322/pUC origin of replication (ori), aminoglycoside phosphotransferase which confers resistance to kanamycin, and the left border repeat from nopaline C58 T-DNA (LB-T-DNA repeat). Additionally, ARDV-02 contains the plant selection marker cassette (StUbi3P:Csr1StUbi3T) which confers resistance to imazapyr, and the cassette with the genes of interest (GmSeed2:sig2:OBC-T4:FM:OaS1-T2:FM:OaS1-T:FM:OBC-T2:AtHSP T:AtUbi10T) which are located between the LB and RB T-DNA repeats.
Organism: Glycine Max.
Mode of transformation: Agrobacterium tumefaciens, disarmed.
Phenotype description Herbicide Tolerance—imazapyr resistant; Product Quality −[Storage protein altered].

### AHAS Selection Marker Cassette

Genotype: Selection Marker
Promoter: [StUbi3P]; Donor: [Solanum tuberosum]; Description: [ubiquitin 3 gene to promote expression of imazapyr resistance gene].
Gene: [CSR1]; Donor: [Arabidopsis thaliana]; Description: [confer resistance to imazapyr for selection of transgenic events].
Terminator: Name: [StUbi3T]; Donor: [Solanum tuberosum]; Description: [ubiquitin 3 gene for CSR1 transcription stabilization].

An exemplary sequence for the selection marker comprises SEQ ID NO: 905.

### Gene of Interest

Promoter: Name: [GmSeed2]; Donor: [Glycine max]; Description: [Promoter from the 5′ region of glycinin Gy1 to promote expression of gene of interest in soybean seed].

Signal Peptide: Name: [sig2]; Donor: [Glycine max]; Description: [Glycinin G1N-terminal peptide to localize heterologous protein].

Gene: Name: [OBC-T4:FM:OaS1-T2:FM:OaS1-T:FM:OBC-T2]; Donor:[Bos taurus] Description: [Soybean codon-optimized bovine beta-casein from Bos taurus encoding casein protein tagged with two amino acids (amino acids 126-127) from soybean codon-optimized bovine kappa-casein from Bos taurus encoding a native chymosin cleavage site, tagged with soybean codon-optimized bovine alphaS1-casein from Bos taurus encoding casein protein, tagged with two amino acids (amino acids 126-127) from soybean codon-optimized bovine kappa-casein from Bos taurus encoding a native chymosin cleavage site, tagged with soybean codon-optimized bovine alpha-casein from Bos taurus encoding casein protein, tagged with two amino acids (amino acids 126-127) from soybean codon-optimized bovine kappa-casein from Bos taurus encoding a native chymosin cleavage site, tagged with soybean codon-optimized bovine beta-casein from Bos taurus encoding casein protein].

Terminator: Name: [AtHSP_T:AtUbi10_T]; Donor: [Arabidopsis thaliana]; Description: [Heat shock termination sequence from Arabidopsis thaliana followed by ubiquitin 10 termination sequence from Arabidopsis thaliana to stabilize gene of interest transcript].

### Construct 2: Beta-casein-Beta-casein-Kappa-casein-Beta-lactoglobulin

ARDV-11 contains the binary pCAMBIA3300 base vector with features such as the right border repeat from nopaline C58 T-DNA, (RB-T-DNA repeat) the stability protein from Pseudomonas plasmid pVS1 (pVS1 StaA), the replication protein from Pseudomonas plasmid pVS1 (pVS1 repA), origin of replication for the Pseudomonas plasmid pVS1 (pVS1 oriV), the basis of mobility region from pBR322 (bom), high-copy-number ColEl/pMB1/pBR322/pUC origin of replication (ori), aminoglycoside phosphotransferase which confers resistance to kanamycin, and the left border repeat from nopaline C58 T-DNA (LB-T-DNA repeat). Additionally, ARDV-11 contains the plant selection marker cassette (StUbi3P:Csr1StUbi3T) which confers resistance to imazapyr, and the cassette with the genes of interest (GmSeed2:sig2:OBC-T3:OBC-T2:OKC1-T:OLG1:AtHSP T:AtUbi10T) which are located between the LB and RB T-DNA repeats.
Organism: Glycine Max
Mode of transformation: [Agrobacterium tumefaciens, disarmed].
Phenotype description: Herbicide Tolerance—imazapyr resistant; Product Quality-[Storage protein altered].

### AHAS Selection Marker Cassette

Genotype: Selection Marker Promoter: [StUbi3P]; Donor: [Solanum tuberosum]; Description: [ubiquitin 3 gene to promote expression of imazapyr resistance gene].
Gene: [CSR1]; Donor: [Arabidopsis thaliana]; Description: [confer resistance to imazapyr for selection of transgenic events].
Terminator: Name: [StUbi3T]; Donor: [Solanum tuberosum]; Description: [ubiquitin 3 gene for CSR1 transcription stabilization].

SEQ ID NO: 907 shows a nucleic acid sequence for an exemplary selection marker.

### Gene(s) of Interest

Promoter: Name: [GmSeed2]; Donor: [Glycine max]; Description: [Promoter from the 5′ region of glycinin Gy1 to promote expression of gene of interest in soybean seed].
Signal Peptide: Name: [sig2]; Donor: [Glycine max]; Description: [Glycinin G1 N-terminal peptide to localize heterologous protein].
Gene: Name:[OBC-T3:OBC-T2:OKC1-T:OLG1]; Donor:[Bos taurus]; Description: [Soybean codon-optimized bovine beta-casein from Bos taurus encoding casein protein tagged with soybean codon-optimized bovine beta-casein from Bos taurus encoding casein protein, tagged with soybean codon-optimized bovine kappa-casein from Bos taurus encoding casein protein, and tagged with soybean codon-optimized beta-lactoglobulin from Bos taurus encoding whey protein].
Terminator: Name: [AtHSP_T:AtUbi10_T]; Donor: [Arabidopsis thaliana]; Description: [Heat shock termination sequence from Arabidopsis thaliana followed by ubiquitin 10 termination sequence from Arabidopsis thaliana to stabilize gene of interest transcript].

### Construct 3: Beta-casein-Beta-casein-Beta-casein-Beta-casein

ARDV-12 contains the binary pCAMBIA3300 base vector with features such as the right border repeat from nopaline C58 T-DNA, (RB-T-DNA repeat) the stability protein from Pseudomonas plasmid pVS1 (pVS1 StaA), the replication protein from Pseudomonas plasmid pVS1 (pVS1 repA), origin of replication for the Pseudomonas plasmid pVS1 (pVS1 oriV), the basis of mobility region from pBR322 (bom), high-copy-number ColEl/pMB1/pBR322/pUC origin of replication (ori), aminoglycoside phosphotransferase which confers resistance to kanamycin, and the left border repeat from nopaline C58 T-DNA (LB-T-DNA repeat). Additionally, ARDV-12 contains the plant selection marker cassette (StUbi3P:Csr1StUbi3T) which confers resistance to imazapyr, and the cassette with the genes of interest (GmSeed2:sig2:OBC-T5:OBC-T4:OBC-T3:OBC-T2:AtHSP T:AtUbi10T) which are located between the LB and RB T-DNA repeats.
Organism: Glycine Max
Mode of transformation: [Agrobacterium tumefaciens, disarmed]
Phenotype description: Herbicide Tolerance—imazapyr resistant; Product Quality-[Storage protein altered]

### AHAS Selection Marker Cassette

Genotype: Selection Marker comprises the sequence of SEQ ID NO: 909.
Promoter: [StUbi3P]; Donor: [Solanum tuberosum]; Description: [ubiquitin 3 gene to promote expression of imazapyr resistance gene].
Gene: [CSR1]; Donor: [Arabidopsis thaliana]; Description: [confer resistance to imazapyr for selection of transgenic events].
Terminator: Name: [StUbi3T]; Donor: [Solanum tuberosum]; Description: [ubiquitin 3 gene for CSR1 transcription stabilization].

### Gene(s) of Interest

Promoter: Name: [GmSeed2]; Donor: [Glycine max]; Description: [Promoter from the 5′ region of glycinin Gy1 to promote expression of gene of interest in soybean seed].
Signal Peptide: Name: [sig2]; Donor: [Glycine max]; Description: [Glycinin G1 N-terminal peptide to localize heterologous protein].
Gene: Name: [OBC-T5:OBC-T4:OBC-T3:OBC-T2]; Donor:[Bos taurus]; Description: [Soybean codon-optimized bovine beta-casein from Bos taurus encoding casein protein tagged with soybean codon-optimized bovine beta-casein from Bos taurus encoding casein protein, tagged with soybean codon-optimized bovine beta-casein from Bos taurus encoding casein protein, and tagged with soybean codon-optimized bovine beta-casein from Bos taurus encoding casein protein].
Terminator; Name: [AtHSP_T:AtUbi10_T]; Donor: [Arabidopsis thaliana]; Description: [Heat shock termination sequence from Arabidopsis thaliana followed by ubiquitin 10 termination sequence from Arabidopsis thaliana to stabilize gene of interest transcript].

### Construct 4: Gamma-Zein-Beta-casein

ARDV-16 contains the binary pCAMBIA3300 base vector with features such as the right border repeat from nopaline C58 T-DNA, (RB-T-DNA repeat) the stability protein from Pseudomonas plasmid pVS1 (pVS1 StaA), the replication protein from Pseudomonas plasmid pVS1 (pVS1 repA), origin of replication for the Pseudomonas plasmid pVS1 (pVS1 oriV), the basis of mobility region from pBR322 (bom), high-copy-number ColEl/pMB1/pBR322/pUC origin of replication (ori), aminoglycoside phosphotransferase which confers resistance to kanamycin, and the left border repeat from nopaline C58 T-DNA (LB-T-DNA repeat). Additionally, ARDV-16 contains the plant selection marker cassette (StUbi3P:Csr1StUbi3T) which confers resistance to imazapyr, and the cassette with the genes of interest (GmSeed2:sig2:γZein:OBC-T2:EUT:Rb7T) which are located between the LB and RB T-DNA repeats.
Organism: Glycine Max
Mode of transformation: [Agrobacterium tumefaciens, disarmed].
Phenotype description: Herbicide Tolerance—imazapyr resistant; Product Quality-[Storage protein altered].

### AHAS Selection Marker Cassette

Genotype: Selection Marker comprising the sequence of SEQ ID NO: 911.
Promoter: [StUbi3P]; Donor: [Solanum tuberosum]; Description: [ubiquitin 3 gene to promote expression of imazapyr resistance gene].
Gene: [CSR1]; Donor: [Arabidopsis thaliana]; Description: [confer resistance to imazapyr for selection of transgenic events].
Terminator: Name: [StUbi3T]; Donor: [Solanum tuberosum]; Description: [ubiquitin 3 gene for CSR1 transcription stabilization].

### Gene(s) of Interest

Promoter: Name: [GmSeed2]; Donor: [Glycine max]; Description: [Promoter from the 5′ region of glycinin Gy1 to promote expression of gene of interest in soybean seed].
Signal Peptide: Name: [sig2]; Donor: [Glycine max]; Description: [Glycinin G1 N-terminal peptide to localize heterologous protein].
Gene: Name: [γZein:OBC-T2]; Donor:[Bos taurus, Zea Mays]; Description: [Soybean codon-optimized Gamma zein from Zea Mays encoding a glutelin protein tagged with soybean codon-optimized bovine beta-casein from Bos taurus encoding casein protein].
Terminator: Name: [NtEU_T:NtRb7_T]; Donor: [Nicotiana tabacum]; Description: [Extensin termination sequence from Nicotiana tabacum followed by Rb7 matrix attachment region termination sequence from Nicotiana tabacum to stabilize gene of interest transcript].

TABLE 32
Exemplary fusion protein constructs

| | | | Gene of Interest |
|---|---|---|---|
| Drawing | Name | Construct Details | (GOI) Payload |
| FIG. | ARDV-02 | GmSeed2:sig2:OBC- | Beta-casein--FM-- |
| 40A | | T4:FM:OaS1-T2:FM:OaS1- | AlphaS1-casein--FM-- |
| | | T:FM:OBC-T2:AtHSPT:AtUbi10T | AlphaS1-casein--FM-- |
| | | | Beta-casein |
| | | | (SEQ ID NO: 906) |
| FIG. | ARDV-11 | GmSeed2:sig2:OBC-T3:OBC- | Beta-casein--Beta- |
| 40B | | T2:OKC1-T:OLG1:AtHSPT:AtUbi10T | casein-Kappa-casein-- |
| | | | Beta-lactoglobulin |
| | | | (SEQ ID NO: 908) |
| FIG. | ARDV-12 | GmSeed2:sig2:OBC-T5:OBC- | Beta-casein--Beta- |
| 40C | | T4:OBC-T3:OBC-T2:AtHSPT:AtUbi10T | casein--Beta-casein-- |
| | | | Beta-casein |
| | | | (SEQ ID NO: 910) |
| FIG. | ARDV-16 | GmSeed2:sig2:yZein:OBC- | Gamma-Zein--Beta- |
| 40D | | T2:EUT:Rb7T | casein |
| | | | (SEQ ID NO: 912) |

### Example 2: Identification of Transgenic Events, Recombinant Protein Extraction and Detection

To quantify recombinant protein expression levels, DNA constructs such as those shown in FIG. 4-FIG. 9 were transformed into soybean using transformation protocols well known in the art, for example, by bombardment or agrobacterium. Total soybean genomic DNA was isolated from the first trifoliate leaves of transgenic events using the PureGene tissue DNA isolation kit (product #158667: QIAGEN, Valencia, CA, USA). Trifoliates were frozen in liquid nitrogen and pulverized. Cells were lysed using the PureGene Cell Lysis Buffer, proteins were precipitated using the PureGene Protein Precipitation Buffer, and DNA was precipitated from the resulting supernatant using ethanol. The DNA pellets were washed with 70% ethanol and resuspended in water.

Genomic DNA was quantified by the Quant-iT PicoGreen (product #P7589: ThermoFisher Scientific, Waltham, MA, USA) assay as described by manufacturer, and 150 ng of DNA was digested overnight with EcoRI, HindIII, NcoI, and/or KpnI, 30 ng of which was used for a BioRad ddPCR reaction, including labelled FAM or HEX probes for the transgene and Lectin1 endogenous gene respectively. Transgene copy number (CNV) was calculated by comparing the measured transgene concentration to the reference gene concentration. A CNV of greater than or equal to one was deemed acceptable.

### Preparation of Total Soluble Protein Samples

Total soluble soybean protein fractions were prepared from the seeds of transgenic events by bead beating seeds (seeds collected about 90 days after germination) at 15000 rpm for 1 min. The resulting powder was resuspended in 50 mM Carbonate-Bicarbonate pH 10.8, 1 mM DTT, 1×HALT Protease Inhibitor Cocktail (Product #78438 ThermoFisher Scientific). The resuspended powder was incubated at 4° C. for 15 minutes and then the supernatant collected after centrifuging twice at 4000 g, 20 min, 4° C. Protein concentration was measured using a modified Bradford assay (Thermo Scientific Pierce 660 nm assay; Product #22660 ThermoFisher Scientific) using a bovine serum albumin (BSA) standard curve.

### Recombinant Protein Quantification via Western Blot Densitometry

SDS-PAGE was performed according to manufacturer's instructions (Product#5678105BioRad, Hercules, CA, USA) under denaturing and reducing conditions. 5 μg of total protein extracts were loaded per lane. For immunoblotting proteins separated by SDS-PAGE were transferred to a PVDF membrane using Trans-Blot® Turbo™ Midi PVDF Transfer Packs (Product #1704157 BioRad) according to manufacturer's guidelines. Membranes were blocked with 3% BSA in phosphate buffered saline with 0.5% Tween-20, reacted with antigen specific antibody and subsequently reacted with fluorescent goat anti rabbit IgG (Product #60871 BioRad, CA). Membranes were scanned according to manufacturer's instructions using the ChemiDoc MP Imaging System (BioRad, CA) and analyzed using ImageLab Version 6.0.1 Standard Edition (Bio-Rad Laboratories, Inc.) Recombinant protein from the seeds of transgenic events was quantified by densitometry from commercial reference protein spike-in standards.

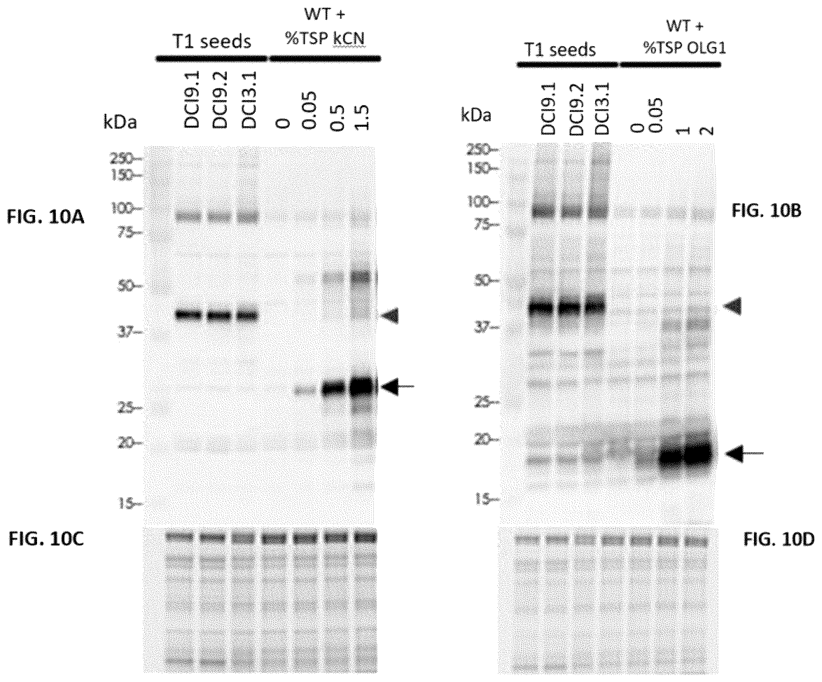

Shown in FIG. 10A, FIG. 10B, FIG. 10C, and FIG. 10D are Western Blots of protein extracted from transgenic soybeans expressing the κ-casein-β-lactoglobulin expression cassette shown in FIG. 4. FIG. 10A shows the fusion protein detected using a primary antibody raised against κ-casein. The first lane is a molecular weight marker. Lanes two (DCI 9.1) and three (DCI 9.2) represent individual seeds from a single transgenic line. Lane four (DCI 3.1) represents a seed from a separate transgenic line. Lane five is protein extracted from wild-type soybean plants, and lanes six—eight are protein extracted from wild-type soybean plants spiked with 0.05% commercial κ-casein (lane 6), 0.5% commercial κ-casein (lane 7), and 1.5% commercial κ-casein (lane 8). The κ-casein commercial protein is detected at an apparent molecular weight (MW) of ˜26 kDa (theoretical: 19 kDa—arrow). The fusion protein is detected at an apparent MW of ˜40 kDa (theoretical: 38 kDa—arrowhead).

FIG. 10B shows the fusion protein detected using a primary antibody raised against β-lactoglobulin. The first lane is a molecular weight marker. Lanes two (DCI 9.1) and three (DCI 9.2) represent individual seeds from a single transgenic line. Lane four (DCI 3.1) represents a seed from a separate transgenic line. Lane five is protein extracted from wild-type soybean plants, and lanes six—eight are protein extracted from wild-type soybean plants spiked with 0.05% commercial β-lactoglobulin (lane 6), 1% commercial β-lactoglobulin (lane 7), and 2% commercial β-lactoglobulin (lane 8). The β-lactoglobulin commercial protein is detected at an apparent MW of ˜18 kDa (theoretical: 18 kDa—arrow). The fusion protein is detected at an apparent MW of ˜40 kDa (theoretical: 38 kDa—arrowhead). FIG. 10C and FIG. 10D show the protein gels as control for equal lane loading (image is taken at the end of the SDS run) for FIG. 10A and FIG. 10B, respectively.

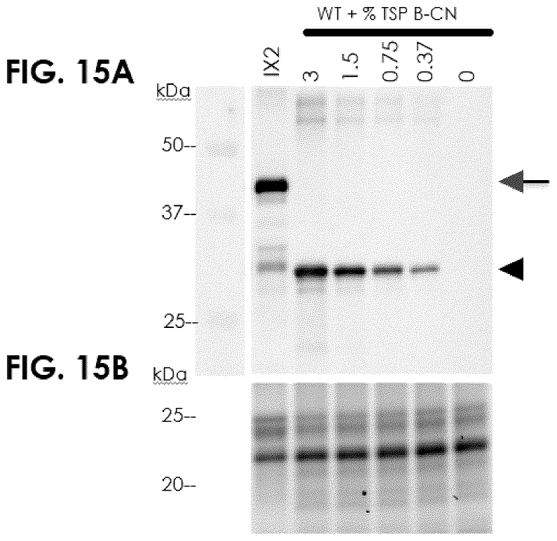

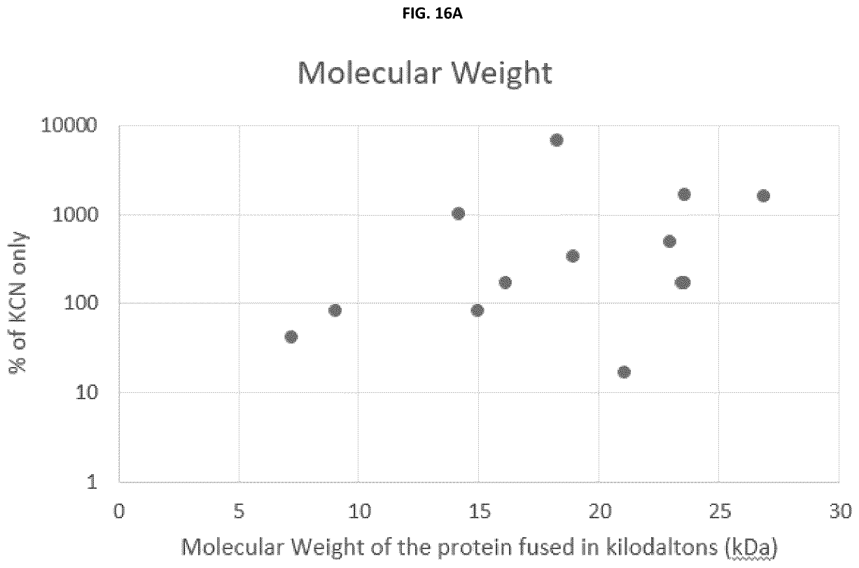

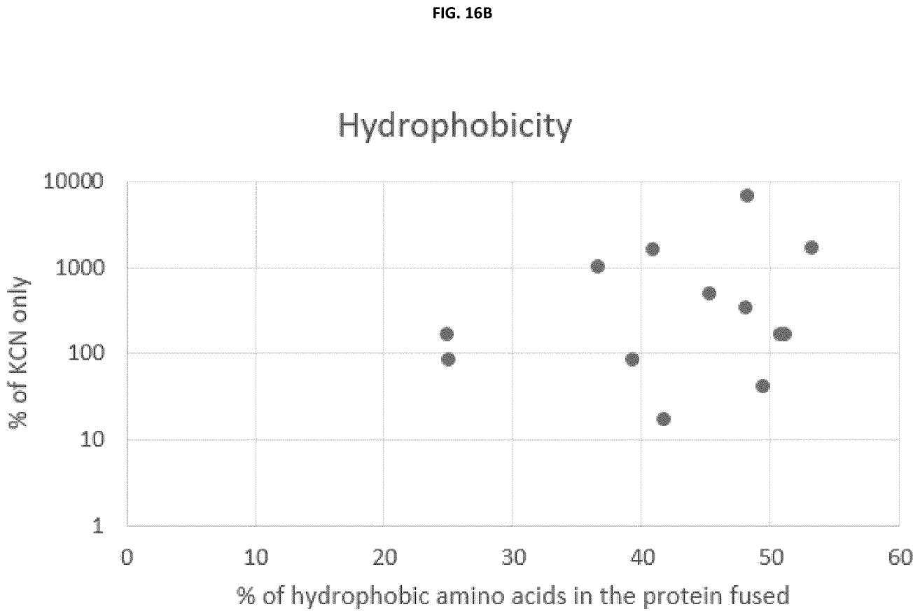

Shown in FIG. 15A and FIG. 15B are Western Blots of protein extracted from transgenic soybeans expressing a β-casein-β-lactoglobulin fusion protein. FIG. 15A shows the fusion protein detected using a primary antibody raised against β-casein. The first lane is a molecular weight marker. Lane two (IX2) represents individual seeds from a single transgenic line. Lanes three through seven are samples comprising protein extracted from wild-type soybean plants spiked with 3% commercial β-casein (lane 3), 1.5% commercial β-casein (lane 4), 0.75% commercial β-casein (lane 5), 0.37% commercial β-casein (lane 6), and 0% commercial β-casein (lane 7). The fusion protein was detected at an apparent MW of ˜40 kDa (arrow; theoretical: 42 kDa).

Other combinations of proteins were tested and evaluated for the percentage of recombinant protein. Cassettes having the same promoter (Seed2-sig), signal peptide (EUT:Rb7T), and in some instances a different terminator, were built with either α-S1-casein, β-casein, κ-casein, or the fusion of β-lactoglobulin (LG) with κ-casein (kCN) (See FIG. 3 and FIG. 8). As shown below in Table 18, none of the cassettes encoding α-S1-casein, β-casein, or κ-casein alone were able to produce expression of the protein at a level that exceeded 1% total soluble protein. However, when κ-casein was fused with β-lactoglobulin, κ-casein was expressed at a level that was greater than 1% total soluble protein. Similarly, when β-casein or alpha-S1-casein were fused with β-lactoglobulin, the β-casein and the alpha-S1-casein were expressed at a level that was greater than 1% total soluble protein.

TABLE 33
Expression levels of milk proteins expressed
alone or in a fusion protein

| | | | Number of events¹ accumulating | |
|---|---|---|---|---|
| | | Total | the recombinant protein at the | |
| | | events¹ | concentration: | |
| | | analyzed | 0-1% TSP | Above 1% TSP |
| Single | κ-Casein | 89 | 89 | 0 |
| Proteins | β-Casein | 12 | 12 | 0 |
| | αS1-Casein | 6 | 6 | 0 |
| Fusion | κ-Casein -LG | 23 | 12 | 11 |
| | β-Casein-LG | 25 | 5 | 20 |
| | αS1-Casein-LG | 10 | 4 | 6 |

¹As used in Table 33, the each “event” refers to an independent transgenic line.

As will be readily understood by those of skill in the art, T-DNA insertion into the plant genome is a random process and each T-DNA lands at an unpredictable genomic position. Thus, for example, each of the 23 events generated in Table 33 for the κ-Casein-LG fusion protein have different genomic insertion loci. The genomic context greatly influences the expression levels of a gene, and each locus will be either favorable or unfavorable for the expression of the recombinant genes. The variability observed at the protein level is a reflection of that random insertion process and explains why 12 out of 23 events present expression levels below 100.

### Example 3: Expression of Casein Multimers

A casein multimer is a fusion protein comprising at least a first casein protein and a second casein protein, wherein the first and second casein proteins are the same (homo-multimer) or different (hetero-multimer). Expression vectors for producing casein multimers were created, using the methods described in Example 1. Specifically, expression vectors were created to express casein multimers comprising: (i) kappa-casein fused to kappa-casein, (ii) kappa-casein fused to beta-casein, and (iii) kappa-casein fused to alpha-S11-casein. Expression vectors were also created to express: (iv) kappa-casein fused to GFP, and (v) kappa-casein fused to beta-lactoglobulin.

Illustrative casein multimers prepared during this study are shown below in Table 34. Colons (:) are used to indicate junctions between various elements of the fusion protein. KDEL indicates the use of a KDEL sequence (i.e., an endoplasmic reticulum retention signal) and FM indicates the use of a linker comprising a chymosin cleavage site.

TABLE 34
Illustrative Milk Protein Multimers

| | | | Amino |
|---|---|---|---|
| | | DNA | Acid |
| | | Sequence | Sequence |
| | Abbreviated | (SEQ | (SEQ |
| Description | Description | ID NO) | ID NO) |
| Optimized para kappa-casein truncated | paraOKC1- | 615 | 616 |
| version 1 (paraOKC1-T):FM:Optimized | T:FM:OLG1 | | |
| beta-lactoglobulin version 1 (OLG1) | | | |
| Optimized para kappa-casein truncated | paraOKC1- | 617 | 618 |
| version 1 (paraOKC1-T):FM:Optimized | T:FM:OLG1:KDEL | | |
| beta-lactoglobulin version 1 | | | |
| (OLG1):KDEL | | | |
| Optimized para kappa-casein truncated | paraOKC1-T:OLG1 | 619 | 620 |
| version 1 (paraOKC1-T):Optimized | | | |
| beta-lactoglobulin version 1 (OLG1) | | | |
| Optimized para kappa-casein truncated | paraOKC1- | 621 | 622 |
| version 1 (paraOKC1-T):Optimized | T:OLG1:KDEL | | |
| beta-lactoglobulin version 1 | | | |
| (OLG1):KDEL | | | |
| Optimized beta-lactoglobulin version 1 | OLG:FM:paraOKC1- | 623 | 624 |
| (OLG1):FM:Optimized para kappa- | T | | |
| casein truncated version 1 (paraOKC1-T) | | | |
| Optimized beta-lactoglobulin version 1 | OLG:FM:paraOKC1- | 625 | 626 |
| (OLG1):FM:Optimized para kappa- | T:KDEL | | |
| casein truncated version 1 (paraOKC1- | | | |
| T):KDEL | | | |
| Optimized beta-lactoglobulin version 1 | OLG:paraOKC1-T | 627 | 628 |
| (OLG1):Optimized para kappa-casein | | | |
| truncated version 1 (paraOKC1-T) | | | |
| Optimized beta-lactoglobulin version 1 | OLG:paraOKC1- | 629 | 630 |
| (OLG1):Optimized para kappa-casein | T:KDEL | | |
| truncated version 1 (paraOKC1- | | | |
| T):KDEL | | | |
| Optimized alpha S1-casein truncated | OaS1-T:FM:OLG1 | 631 | 632 |
| version 1 (OaS1-T):FM:Optimized | | | |
| beta-lactoglobulin version 1 (OLG1) | | | |
| Optimized alpha S1-casein truncated | OaS1- | 633 | 634 |
| version 1 (OaS1-T):FM:Optimized | T:FM:OLG1:KDEL | | |
| beta-lactoglobulin version 1 | | | |
| (OLG1):KDEL | | | |
| Optimized alpha S1-casein truncated | OaS1-T:OLG1 | 635 | 636 |
| version 1 (OaS1-T):Optimized beta- | | | |
| lactoglobulin version 1 (OLG1) | | | |
| Optimized alpha S1-casein truncated | OaS1- | 637 | 638 |
| version 1 (OaS1-T):Optimized beta- | T:OLG1:KDEL | | |
| lactoglobulin version 1 (OLG1):KDEL | | | |
| Optimized beta-lactoglobulin version 1 | OLG1:FM:OaS1-T | 639 | 640 |
| (OLG1):FM:Optimized alpha S1-casein | | | |
| truncated version 1 (OaS1-T) | | | |
| Optimized beta-lactoglobulin version 1 | OLG1:FM:OaS1- | 641 | 642 |
| (OLG1):FM:Optimized alpha S1-casein | T:KDEL | | |
| truncated version 1 (OaS1-T):KDEL | | | |
| Optimized beta-lactoglobulin version 1 | OLG1:OaS1-T | 643 | 644 |
| (OLG1):Optimized alpha S1-casein | | | |
| truncated version 1 (OaS1-T) | | | |
| Optimized beta-lactoglobulin version 1 | OLG1:OaS1- | 645 | 646 |
| (OLG1):Optimized alpha S1-casein | T:KDEL | | |
| truncated version 1 (OaS1-T):KDEL | | | |
| Optimized beta-lactoglobulin version 1 | OLG1:FM:OBC-T2 | 647 | 648 |
| (OLG1):FM:Optimized beta-casein (A2 | | | |
| variant) truncated version 2 | | | |
| Optimized beta-casein (A2 variant) | OBC-T2:OKC1- | 649 | 650 |
| truncated version 2:Optimized kappa- | T:OLG1 | | |
| casein truncated version 1 (OKC1- | | | |
| T):Optimized beta-lactoglobulin version 1 | | | |
| (OLG1) | | | |
| Optimized beta-casein (A2 variant) | OBC-T3:OBC- | 651 | 652 |
| truncated version 3:Optimized beta- | T2:OKC1-T:OLG1 | | |
| casein (A2 variant) truncated version | | | |
| 2:Optimized kappa-casein truncated | | | |
| version 1 (OKC1-T):Optimized beta- | | | |
| lactoglobulin version 1 (OLG1) | | | |
| Optimized beta-casein (A2 variant) | OBC-T4:OBC- | 653 | 654 |
| truncated version 4:Optimized beta- | T3:OBC-T2:OKC1- | | |
| casein (A2 variant) truncated version | T:OLG1 | | |
| 3:Optimized beta-casein (A2 variant) | | | |
| truncated version 2:Optimized kappa- | | | |
| casein truncated version 1 (OKC1- | | | |
| T):Optimized beta-lactoglobulin version 1 | | | |
| (OLG1) | | | |
| Optimized beta-casein (A2 variant) | OBC-T5:OBC- | 655 | 656 |
| truncated version 5:Optimized beta- | T4:OBC-T3:OBC- | | |
| casein (A2 variant) truncated version | T2:OKC1-T:OLG1 | | |
| 5:Optimized beta-casein (A2 variant) | | | |
| truncated version 4:Optimized beta- | | | |
| casein (A2 variant) truncated version | | | |
| 3:Optimized beta-casein (A2 variant) | | | |
| truncated version 2:Optimized para | | | |
| kappa-casein truncated version 1 | | | |
| (paraOKC1-T):Optimized beta- | | | |
| lactoglobulin version 1 (OLG1) | | | |
| Optimized beta-casein (A2 variant) | OBC-T5:OBC- | 657 | 658 |
| truncated version 5:Optimized beta- | T4:OBC-T3:OBC- | | |
| casein (A2 variant) truncated version | T2:OLG1 | | |
| 4:Optimized beta-casein (A2 variant) | | | |
| truncated version 3:Optimized beta- | | | |
| casein (A2 variant) truncated version | | | |
| 2:Optimized beta-lactoglobulin version 1 | | | |
| (OLG1) | | | |
| Optimized beta-casein (A2 variant) | OBC-T5:OBC- | 659 | 660 |
| truncated version 5:Optimized beta- | T4:OBC-T3:OBC-T2 | | |
| casein (A2 variant) truncated version | | | |
| 4:Optimized beta-casein (A2 variant) | | | |
| truncated version 3:Optimized beta- | | | |
| casein (A2 variant) truncated version 2 | | | |
| Optimized beta-casein (A2 variant) | OBC-T5:FM:OBC- | 661 | 662 |
| truncated version 5:FM:Optimized beta- | T4:FM:OBC- | | |
| casein (A2 variant) truncated version | T3:FM:OBC- | | |
| 4:FM:Optimized beta-casein (A2 variant) | T2:FM:OLG1 | | |
| truncated version 3:FM:Optimized beta- | | | |
| casein (A2 variant) truncated version | | | |
| 2:FM:Optimized beta-lactoglobulin version | | | |
| 1 (OLG1) | | | |
| Optimized beta-casein (A2 variant) | OBC-T5:FM:OBC- | 663 | 664 |
| truncated version 5:FM:Optimized beta- | T4:FM:OBC- | | |
| casein (A2 variant) truncated version | T3:FM:OBC-T2 | | |
| 4:FM:Optimized beta-casein (A2 variant) | | | |
| truncated version 3:FM:Optimized beta- | | | |
| casein (A2 variant) truncated version 2 | | | |
| Optimized beta-casein (A2 variant) | OBC-T4:FM:OBC- | 792 | 793 |
| truncated version 4:FM:Optimized beta- | T3:FM:OBC-T2 | | |
| casein (A2 variant) truncated version | | | |
| 3:FM:Optimized beta-casein (A2 variant) | | | |
| truncated version 2 | | | |
| Optimized beta-casein (A2 variant) | OBC-T4:FM:OBC- | 794 | 795 |
| truncated version 4:FM:Optimized beta- | T3:FM:OBC- | | |
| casein (A2 variant) truncated version | T2:FM:OLG1 | | |
| 3:FM:Optimized beta-casein (A2 variant) | | | |
| truncated version 2:FM:Optimized beta- | | | |
| lactoglobulin version 1 (OLG1) | | | |
| Optimized beta-casein (A2 variant) | OBC-T4:OBC- | 796 | 797 |
| truncated version 4:Optimized beta- | T3:OBC-T2 | | |
| casein (A2 variant) truncated version | | | |
| 3:Optimized beta-casein (A2 variant) | | | |
| truncated version 2 | | | |
| Optimized beta-casein (A2 variant) | OBC-T4:OBC- | 798 | 799 |
| truncated version 4:Optimized beta- | T3:OBC-T2:OLG1 | | |
| casein (A2 variant) truncated version | | | |
| 3:Optimized beta-casein (A2 variant) | | | |
| truncated version 2:Optimized beta- | | | |
| lactoglobulin version 1 (OLG1) | | | |

The expression constructs were transformed into soybean, as described in Example 2. Quantification of casein multimer expression was performed using Western Blot Densitometry. Table 35 shows expression levels of the casein proteins when expressed in the indicated multimer constructs, relative to the caseins expressed alone (i.e., not as part of a fusion protein).

TABLE 35
Expression levels of casein multimers relative to caseins expressed alone

| | Fold increase | Fold increase | Fold increase |
|---|---|---|---|
| | in expression | in expression | in expression |
| | relative to κ- | relative to B- | relative to αS1- |
| Casein Multimer Fusion Protein | Casein alone | Casein alone | Casein alone |
| κ-Casein:κ-Casein | 3.4 | — | — |
| κ-Casein:β-Casein | 17 | 2.5 | — |
| κ-Casein:αS1-Casein | 5 | — | 32 |
| κ-Casein:GFP | 16 | — | — |
| αS1-Casein:GFP | — | — | 77 |
| κ-Casein:β-Lactoglobulin | 68 | — | — |
| β-Casein:β-Lactoglobulin | — | 27 | — |
| αS1-Casein-β:Lactoglobulin | — | — | 522 |
| κ-Casein:α-Lactalbumin | 10 | — | — |
| β-casein:α-Lactalbumin | — | 2.8 | — |
| αS1-Casein:α-Lactalbumin | — | — | 150 |
| β-Casein:β-Casein:β-Casein | — | 10.7 | — |
| β-Casein:β-Casein:β-Casein:β-Casein | — | 14.5 | — |

As shown in Table 35, expression of the casein proteins as multimers led to significant increases in expression relative to the caseins expressed alone. Specifically, expression of kappa-casein as a casein homo-multimer led to a 3.4-fold increase in expression relative to expression of casein alone. Expression of kappa-casein as a multimer with beta-casein led to 17-fold and 2.5-fold increases in expression, respectively, relative to either protein expressed alone. Expression of kappa-casein as a multimer with alpha-S1-casein led to 5-fold and 32-fold increases in expression, respectively, relative to either protein expressed alone. Expression of kappa-casein fused to GFP led to a 16-fold increase in expression. Expression of kappa-casein fused to beta-lactoglobulin led to a 68-fold increase in expression, and expression of beta-casein fused to beta-lactoglobulin led to an 11.5-fold increase in expression. Expression of beta-casein or alpha-S-casein was also increased by fusion to alpha-lactalbumin (2.8-fold and 150-fold respectively).

Expression of β-casein as a trimer or tetramer also led to significant increases in expression relative to β-casein expressed alone (18-fold and 18.5-fold, respectively).

Without being bound by any theory, it is believed that fusing a first casein protein to a second protein partially or fully shields each of the proteins from degradation by host cell proteases and allows for accumulation of the casein in the cell.

### Example 4: Kappa-Casein is Sensitive to Soybean Endogenous Proteolysis Activity

To determine whether endogenous host cell proteases are responsible for degradation of casein proteins expressed alone, soybean total protein extracts were spiked with 100 ng of commercial kappa-casein, in the presence or absence of Halt Protease Inhibitor Cocktail (Thermo Fisher Scientific). All samples were incubated at 37° C. for two hours. The samples were then subjected to analysis using a Western blot. The protein was detected using a primary antibody against kappa-casein.

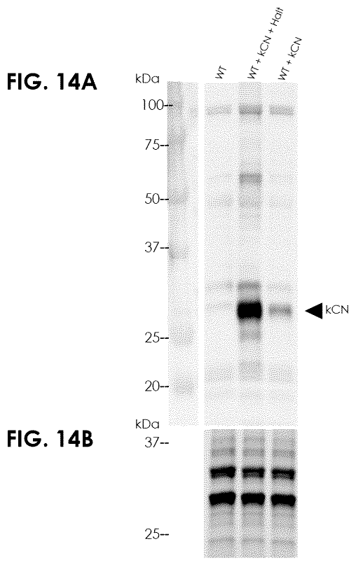

As shown in FIG. 14A and FIG. 14B, most of the kappa-casein added to the cellular extracts was degraded, and this degradation was prevented by the addition of protease inhibitors. This data confirms that kappa-casein is sensitive to soybean endogenous proteolysis activity. Inhibition of endogenous proteolysis activity may lead to increased casein accumulation in transformed cells.

### Example 5: Food Compositions

The transgenic plants expressing the recombinant fusion proteins described herein can produce milk proteins for the purpose of food industrial, non-food industrial, pharmaceutical, and commercial uses described in this disclosure. Illustrative methods for making a food composition are provided in FIG. 13 and FIG. 17.

A fusion protein comprising an unstructured milk protein (e.g., a casein such as para-κ-casein, κ-casein, β-casein, αS1-casein, or αS2-casein), and a structured mammalian protein (e.g. β-lactoglobulin) is expressed in a transgenic plant (e.g. a soybean plant). In some constructs, the fusion protein comprises a chymosin cleavage site between the milk protein (e.g., a casein such as para-κ-casein, κ-casein, β-casein, αS1-casein, or αS2-casein) and the β-lactoglobulin.

The fusion protein is extracted from the plant. The fusion protein is then treated with chymosin, to separate the milk protein (e.g., a casein) from the β-lactoglobulin. The casein is isolated and/or purified and used to make a food composition (e.g., cheese).

### Example 6: Determination of Physicochemical Parameters that Contribute to Casein Accumulation in Plants

The purpose of the experiments described in this example was to determine the physicochemical parameters of proteins (i.e., fusion partners) that, when fused to a casein protein, are capable of enhancing accumulation thereof.

Various proteins having distinct physicochemical properties were fused to kappa-casein. The physicochemical properties thereof are listed in Table 36. The fusion proteins were then expressed in soybean plants as described above. Protein expression levels of the fusion protein and relative increases thereof relative to casein alone (not expressed as a fusion) were measured.

Results are summarized in Table 36. The term “KCN-fusion % TSP” refers to protein expression levels of the fusion protein, as a fraction of total soluble protein. The term “% KCN only” refers to increases in kappa-casein expression relative to kappa casein expressed alone (not as a fusion). The % KCN only value was calculated by division the KCN-fusion % TSP value by 0.059 (i.e., the percent accumulation of kappa-casein by itself).

TABLE 36
Proteins fused to kappa casein and physicochemical parameters thereof

| | | | Percentage | Number of | | |
|---|---|---|---|---|---|---|
| | Uniprot | | hydrophobic | disulfide | KCN- | |
| | Accession | MW in | AA/Total | bonds/per | fusion | % KCN |
| Full name | No. | kDa | AA (%) | 10 kDa | % TSP | only |
| Kappa Casein | P02668 | 18.9 | 48.04 | 0.53 | 0.2 | 339 |
| Beta Casein | P02668 | 23.5 | 53.11 | 0 | 1 | 1695 |
| Alpha Casein | P18626 | 22.9 | 45.23 | 0 | 0.29 | 492 |
| Beta Lactoglobulin | P02754 | 18.2 | 48.15 | 1.1 | 4 | 6780 |
| Alpha Lactalbumin | P00711 | 14.1 | 36.59 | 2.2 | 0.34 | 1017 |
| Green Fluorescent | P42212 | 26.8 | 40.76 | 0 | 0.94 | 1593 |
| Protein | | | | | | |
| Lysozyme | Q6B411 | 14.9 | 39.23 | 2.68 | 0.05 | 85 |
| 2S globulin | P19594 | 16.1 | 24.82 | 2.48 | 0.1 | 169 |
| Oleosin A | P29530 | 23.5 | 51.11 | 0 | 0.1 | 169 |
| Oleosin B | P29531 | 23.4 | 50.67 | 0 | 0.1 | 169 |
| Kunitz-Trypsin | Q39898 | 21 | 41.67 | 0.95 | 0.001 | 16.9 |
| inhibitor | | | | | | |
| Bowman-Birk | I1MQD2 | 9 | 25 | 3.33 | 0.05 | 85 |
| inhibitor | | | | | | |
| Hydrophobin II | P79073 | 7.19 | 49.3 | 5.56 | 0.025 | 42 |

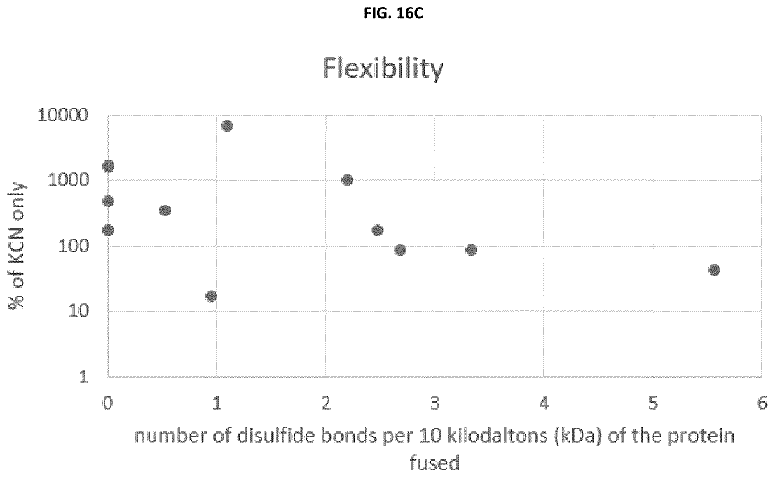

An analysis of the data shown in Table 36 is provided in FIG. 16A, FIG. 16B, and FIG. 16C. This analysis suggests that there are several physicochemical properties of proteins that when fused to kappa-casein, may contribute to accumulation of the kappa-casein. The first is molecular weight. In general, a protein (fusion partner) with molecular weight of 15 kDa or higher tended to increase accumulation (FIG. 16A). The second is hydrophobicity. A protein (fusion partner) having greater than about 30% hydrophobic amino acids also tended to increase accumulation (FIG. 16B). The third is flexibility. A protein (fusion partner) with less than about 2.5 disulfide bonds per 10 kDa molecular weight also tended to increase accumulation (FIG. 16B). The disulfide bonds were predicted using a computer program. Notably, the number of cysteines in the protein, on its own, was not predictive of the protein's ability to contribute to accumulation of the kappa-casein.

Notably, as evidenced by the data in Table 36 and FIG. 16A-16C, the fusion partner did not need to have all three of these characteristics in order to increase accumulation of kappa-casein. For example, increases in accumulation were observed in some cases where the fusion partner had only one, only two or all three of these characteristics.

### Example 7: Fusion Proteins Comprising Milk Proteins and Prolamin Proteins

To determine the impact of including a prolamin in a fusion protein on accumulation thereof in a seed, expression vectors for producing fusion proteins comprising a milk protein and a prolamin protein were created using the methods described in Example 1. Specifically, expression vectors were created to express fusion proteins comprising: (i) canein (gCan27) fused to β-casein, (ii) zein (7-zein) fused to β-casein, and (iii) canein (gCan27) fused to κ-casein.

Illustrative fusion proteins used during this study are shown below in Table 22. Colons (:) are used to indicate junctions between various elements of the fusion protein. FM indicates the use of a linker comprising a chymosin cleavage site.

TABLE 37
Fusion Proteins Comprising a Prolamin

| | | | Amino |
|---|---|---|---|
| | | DNA | Acid |
| | | Sequence | Sequence |
| | Abbreviated | (SEQ | (SEQ |
| Description | Description | ID NO) | ID NO) |
| 27 kD gamma canein | gCan27:FM:OBC- | 802 | 803 |
| (gcan27):FM:Optimized beta casein | T2 | | |
| truncated version 2 (OBC-T2):FM | | | |
| Gamma zein (yZein):Optimized beta-casein | yZein:OBC-T2 | 806 | 807 |
| truncated version 2 (OBC-T2) | | | |

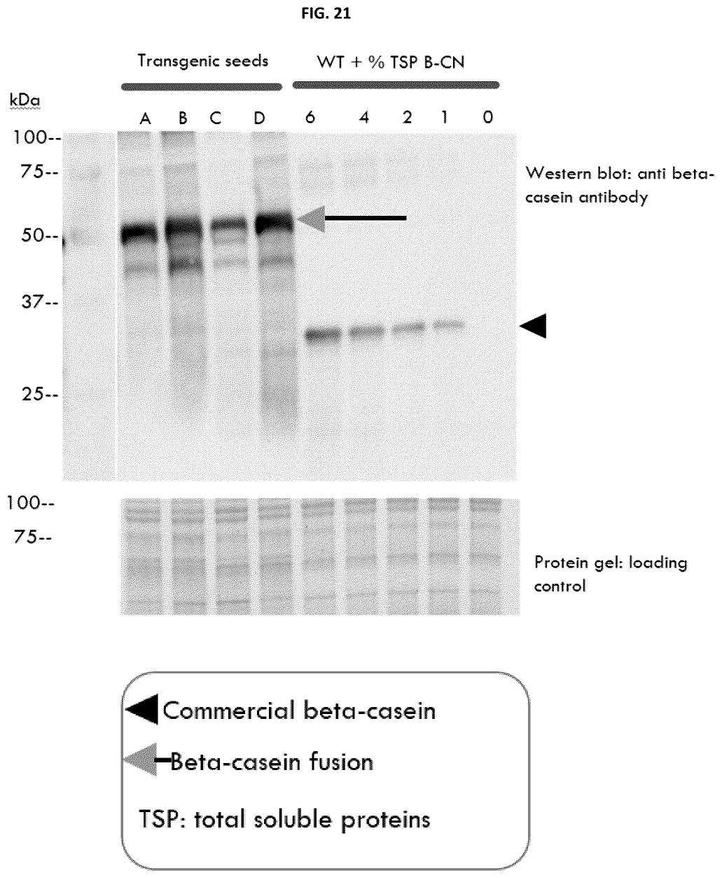

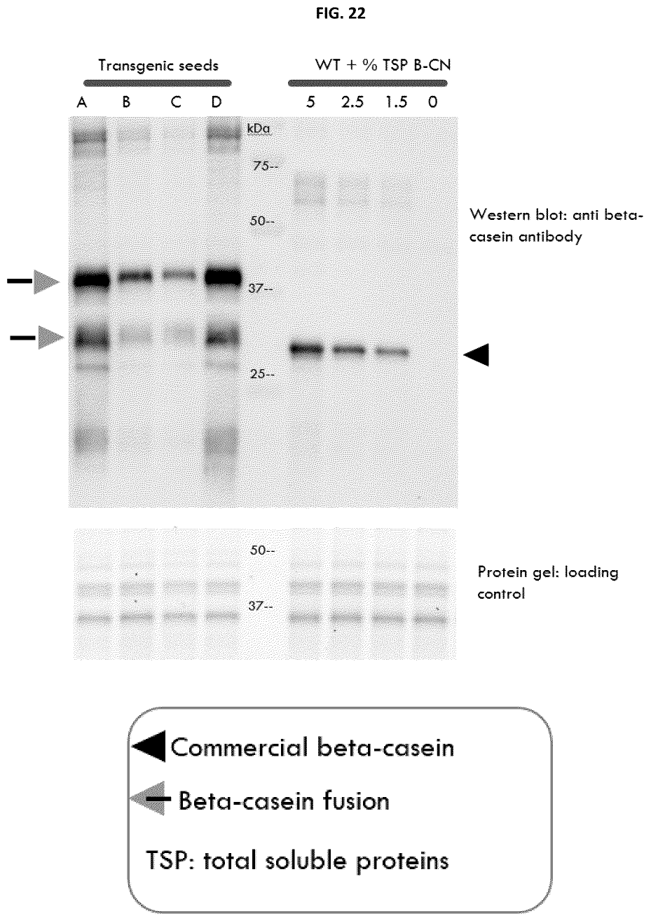

The expression constructs were transformed into soybean, as described in Example 2. Western blots showing detection ofbeta-casein in transgenic seed extracts are provided in FIG. 21 and FIG. 22. Quantification of casein multimer expression was performed using Western Blot Densitometry. Table 38 shows expression levels ofthe beta-casein protein when expressed in the indicated fusion constructs, relative to the beta-casein expressed alone (i.e., not as part ofa fusion protein).

TABLE 38
Expression levels of beta-casein when fused to
a prolamin relative to caseins expressed alone

| | Fold increase in | Fold increase in |
|---|---|---|
| Casein Multimer | expression relative | expression relative |
| Fusion Protein | to κ-Casein alone | to B-Casein alone |
| gCan27:κ-Casein | 16 | — |
| gCan27:β-Casein | — | 40 |
| Zein:β-Casein | — | 55 |

As shown in Table 38, fusion of caseins to either canein or zein led to significant increases in expression relative to the caseins expressed alone. Specifically, expression of kappa-casein fused to gCan27 led to a 16-fold increase in expression relative to expression of kappa-casein alone. Expression of beta-casein fused to gCan27 led to a 40-fold increase in expression, relative to beta-casein expressed alone. Fusion of beta-casein to zein led to a 55-fold increase in expression, relative to beta-casein expressed alone. In each of these experiments, the casein protein accumulated in the seeds at a level well above 1% TSP.

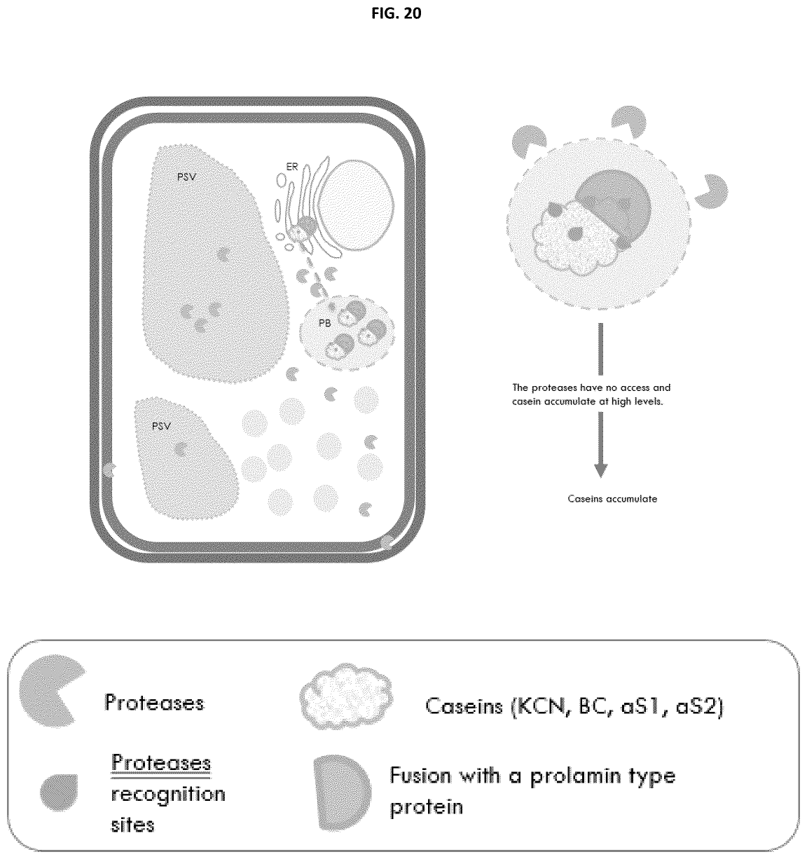

Without being bound by any theory, it is believed that fusing a casein protein to a prolamin (e.g., a canein or a zein) leads to the formation of a protein body in the seed. The casein is then sequestered in the protein body, which partially or fully shields the casein from degradation by host cell proteases and allows for accumulation of the casein in the cell. An illustrative mechanism for protein body formation is found in FIG. 20.

### Example 8: Phosphorylation Prevents Degradation of Caseins in a Plant Cell

It was hypothesized that various post-translational modifications, such as phosphorylation, may have a “shielding” effect which prevents degradation of milk proteins, especially casein proteins, in a plant cell. Specifically, it was hypothesized that by adding one or more phosphates to a casein protein expressed in a plant cell, it may be possible to block and/or reduce the access of plant proteases to various cleavage sites on the protein. By reducing the ability of the plant proteases to degrade the milk protein, higher levels of protein accumulation may be possible.

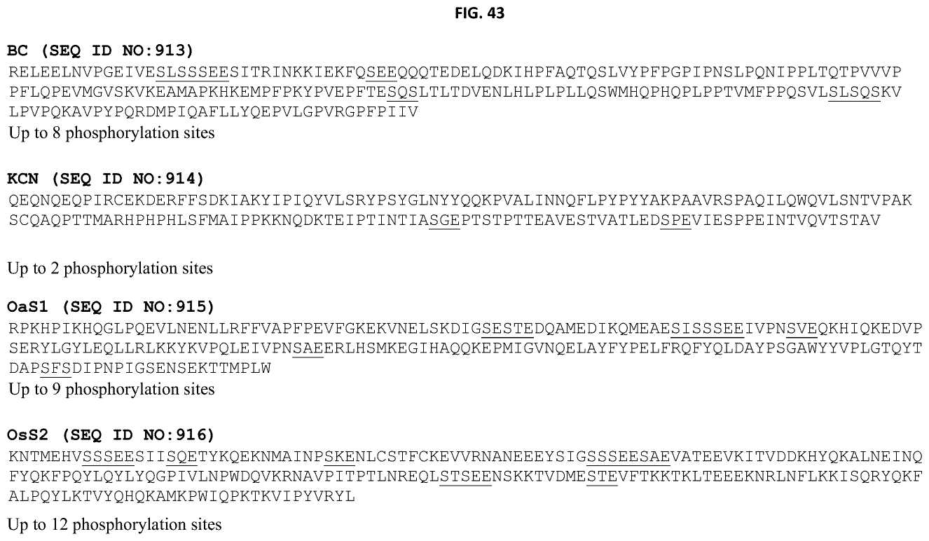

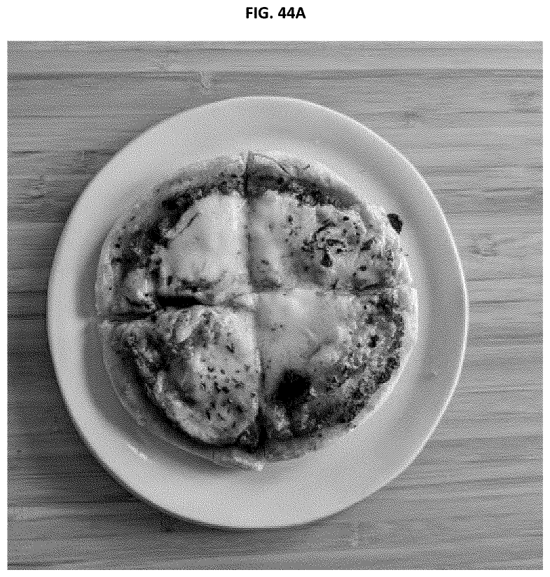

To test this hypothesis, the enzyme Fam20C was co-expressed with one or more caseins in a plant cell. Fam20C is a golgi localized serine kinase and is responsible for the phosphorylation of caseins (Bauman, D. E., et al. “Major advances associated with the biosynthesis of milk.” Journal of dairy science 89, no. 4 (2006): 1235-1243.). These kinases recognize a S—X—S or a S-X-E motif To identify locations of identifiable motifs, ScanProsite was employed to search for the motif sequences of interest in caseins. Using the SxE/pS motify, Fam20C can account for approximately two-thirds of the secreted phosphoproteome. Fam20A is a pseudokinase (not catalytically active) that binds with Fam20C and makes it more active. Based on the analysis, the predicted phosphorylated sites in beta-casein, kappa-casein, OaS1, and OsS2, are shown in FIG. 43. Other kinases that can phosphorylate caseins are provided in Table 39. Based on InterPro, kinases that belong to the complementary families are: IPR024869 (FAM20), IPR009581 (FAM20_C), IPRO11009 (Kinase-like_dom_sf), IPR000719 (Prot_kinase_dom), IPRO17441 (Protein_kinase_ATP_BS), IPR008271 (Ser/Thr_kinase_AS), and IPR022247 (Casein_kinase-1_gamma_C).

TABLE 39
Exemplary kinases that can phosphorylate caseins.

| Kinases family | Motif | Reference |
|---|---|---|
| Fam20C (aka Golgi | S-X-S/pE | Tagliabracci V S, Wiley S E, Guo X, Kinch L N, |
| casein Kinase, (G- | | Durrant E, Wen J, Xiao J, Cui J, Nguyen K B, Engel |
| CK), dentin matrix | | J L, Coon J J, Grishin N, Pinna L A, Pagliarini D J, |
| protein-4 (DMP-4)) | | Dixon J E. A Single Kinase Generates the Majority |
| | | of the Secreted Phosphoproteome. Cell. 2015 Jun. |
| | | 18; 161(7): 1619-32. doi: |
| | | 10.1016/j.cell.2015.05.028. PMID: 26091039; |
| | | PMCID: PMC4963185. |
| Fam20C/Fam20A | S-X-S/pE | Tagliabracci V S, Wiley S E, Guo X, Kinch L N, |
| pseudokinase | | Durrant E, Wen J, Xiao J, Cui J, Nguyen K B, Engel |
| | | J L, Coon J J, Grishin N, Pinna L A, Pagliarini D J, |
| | | Dixon J E. A Single Kinase Generates the Majority |
| | | of the Secreted Phosphoproteome. Cell. 2015 Jun. |
| | | 18; 161(7): 1619-32. doi: |
| | | 10.1016/j.cell.2015.05.028. PMID: 26091039; |
| | | PMCID: PMC4963185. |
| Casein Kinase 1 | pS/pT-X-X-S/T | Venerando A, Ruzzene M, Pinna L A. Casein |
| (CK1) | (E, D)n-X-X- | kinase: the triple meaning of a misnomer. Biochem |
| | S/T | J. 2014 Jun. 1; 460(2): 141-56. doi: |
| | | 10.1042/BJ20140178. PMID: 24825444. |
| Casein Kinase 2 | S/T-X-X- | Venerando A, Ruzzene M, Pinna L A. Casein |
| (CK2) | E/D/pS/pY | kinase: the triple meaning of a misnomer. Biochem |
| | | J. 2014 Jun. 1; 460(2): 141-56. doi: |
| | | 10.1042/BJ20140178. PMID: 24825444. |
| FAMK-1 | S-X-S/pE | pnas.org/doi/10.1073/pnas.1309211110 |
| Casein kinase-like | serine/threonine | tandfonline.com/doi/full/10.1080/17429145.2012.7 |
| proteins (kinases in | protein kinase - | 49433&sa=D&source=docs&ust=16626909853926 |
| plants that can | motif not | 88&usg=AOvVaw34iotcnEHRrMNy2c_ZbqbA |
| phosphorylate | specified | |
| caseins) | | |
| Early Flowering1 | serine/threonine | Dai and Xue, 2010; Chen et al., 2018 |
| (EL1)-like proteins | protein kinase - | |
| (kinases in plants that | motif not | |
| can phosphorylate | specified | |
| caseins) | | |

The phosphorylatable residue(s) are in bold and the specificity determinants are underlined. X denotes any residue, but in the CK2 consensus, these are preferably acidic amino acids.

The expression construct used in this study is shown in FIG. 24E. The construct comprised (i) a first expression cassette comprising the GmSeed2 promoter (SEQ ID NO: 813), a sig2 signal peptide (SEQ ID NO: 814), a sequence encoding a fusion protein (GOI, see table below), and an AtHSP/AtUbi10 Terminator (SEQ ID NO: 815, 816), and (ii) a second expression cassette comprising the pvPhas promoter (SEQ ID NO: 817), an Arc5′UTR (SEQ ID NO: 818), a sig10 signal peptide (SEQ ID NO: 819), a sequence encoding the Fam20c kinase (SEQ ID NO: 821), and a 3 arc terminator (SEQ ID NO: 822). This expression construct was cloned into a binary Agrobacterium vector, as illustrated in FIG. 23. The vector was then transformed into soybean plants, and protein expression was measured in the seeds using a Western Blot. An anti-beta-casein antibody was used to detect fusion protein expression.

### Results

Expression levels of the transformants are shown in Table 40. Table 40 compares expression levels of the casein when expressed alone vs. as a fusion protein, with or without Fam20c co-expression. When expressed without the kinase, the kappa-casein:beta-casein fusion protein produced a 17-fold increase in kappa-casein expression relative to kappa-casein expressed alone, and a 2.5-fold increase in beta-casein expression relative to beta-casein expressed alone. When this fusion protein was co-expressed with Fam20C, the expression of kappa-casein was 254-fold greater than kappa-casein expressed alone, and 38-fold greater than beta-casein expressed alone. Notably, expression of a kappa-casein:beta-casein:alpha-S1-casein fusion with a kinase resulted in a 185-fold increase in kappa-casein relative to kappa-casein alone, 25-fold increase in expression of beta-casein relative to beta-casein alone, and 1000-fold increase in alpha-S1-casein relative to alpha-S1-casenin alone.

TABLE 40
Expression levels of caseins when co-expressed with
a kinase compared to caseins expressed alone

| | | Increased | Increased | Increased |
|---|---|---|---|---|
| | | fold vs KCN | fold vs BCN | fold vs aS1 |
| No | κ-Casein-B-Casein | 17 | 2.5 | — |
| Kinase | | | | |
| Kinase | κ-Casein-B-Casein/ | 254 | 38 | — |
| | Fam20C | | | |
| | κ-Casein-B-Casein- | 185 | 25 | 1000 |
| | aS1-Casein/Fam20C | | | |

Taken together, these data indicate that co-expression of a kinase with a fusion protein comprising one or more casein proteins in a plant cell leads to an increase in accumulation of the casein protein in the cell. Without being bound by any theory, the addition of one or more phosphates to the casein protein can protect it from degradation by one or more plant proteases. Prophetic Study Evaluating Performance of Alternate Kinases

A study to evaluate the expression levels of caseins when co-expressed with alternate kinases as compared to casein-alone is performed utilizing any or all of the exemplary kinases from Table 39 or a kinase from a complementary family. An expression construct encoding for an alternate kinase and one or more caseins is cloned into a binary Agrobacterium vector. The vector is transformed into a soybean plant, and protein expression is measured in soybean seeds.

Expression levels are evaluated to determine differences between plants expressing the alternate kinase and those not expressing the alternate kinase. Additionally a comparative study can be done to rank performance between the different kinases.

### Example 9: Fusion to a Glycosylated Peptide to Increase Accumulation of Caseins in a Plant Cell

Certain genetic elements increase the secretion and stability of proteins in plant cells (Jia Li et al., Secretion of Active Recombinant Phytase from Soybean Cell-Suspension Cultures, 1997; Jianfeng Xu et al., High-Yields and Extended Serum Half-Life of Human Interferon a2b Expressed in Tobacco Cells as Arabinogalactan-Protein Fusions, 2007). Many aspects of plant growth involve hydroxyproline (Hyp)-rich glycoproteins (GRGPs). Accordingly, it was hypothesized that fusion of a casein to a glycoprotein tag could be used to increase accumulation of the casein in a plant cell. Table 41 shows examples of glycotags that can be used in recombinant milk protein production. Additional glycotags can be identified in Johnson, K. L., Cassin, A. M., Lonsdale, A., Bacic, A., Doblin, M. S., & Schultz, C. J. (2017). Pipeline to identify hydroxyproline-rich glycoproteins. Plant Physiology, 174(2), 886-903, see Table 91.

TABLE 41
Exemplary glycotags

| Glyto tag | Description |
|---|---|
| (SP)10 | Synthetic 10 tandem repeats of SP |
| (SP)20 | Synthetic 20 tandem repeats of SP |
| (SP)32 | Synthetic 32 tandem repeats of SP |
| (SP4)18 | Synthetic 18 tandem repeats of SPPPP |
| rabies glycoprotein (rgp) | glycoprotein of rabies virus |
| ANITVNITV | Synthetic glycopeptide |
| ANITVNITV | Synthetic glycopeptide |
| Protein or portion of a protein | |
| considered a hydroxyproline-rich | |
| glycoprotein containing motifs | |
| listed in the “Annotated HRGPs” | |
| tabs | |

SP repeats glycoprotein

In an initial experiment, a glycoprotein comprising 11 tandem SP repeats was identified from a native soybean protein (Glyma.02g204500), annotated as early nodulin-like protein 10. This tag, dubbed the (SP)11 tag, was codon optimized in IDT and fused to the N- or C-terminus of kappa-casein (See FIG. 25A-25C). The (SP)11-kappa-casein was then cloned into a binary Agrobacterium vector, and transformed into soy.

Notably, the expression of (SP)11-kappa-casein in the seeds was increased 13-fold over expression of kappa-casein alone (i.e., not fused with a glycoprotein tag). This data indicates that fusion with a glycoprotein tag can be used to increase accumulation of caseins in a plant cell.

### Prophetic Fusions of Other Disclosed Glycotags

Additional glycotags listed in table 41 will be tested in N- or C-terminal fusions with the milk proteins of the present disclosure. It is expected that fusions of these glycotags will increase expression of the milk protein.

### CD45 M-domain

In a similar experiment, the M domain of CD45 (receptor-type tyrosine-protein phosphatase C) was fused to kappa-casein (alternate caseins can be employed). The M domain is known to function as an ER-retention signal. Briefly, the M-domain from CD45 (Uniprot Accession No. P08575, amino acids Ala231 to Asp 290) was codon optimized using the Glycine max codon usage bias and fused to the N- or C-terminus of kappa-casein. In some constructs, a KDEL sequence was added to the C-terminus of the M-domain or the GOI (see FIG. 25E-25F). The fusion of the M domain to the C-terminus may cause ER retention of the fusion protein, leading to increased accumulation thereof in the cell.

Results of the CD45-Beta casein tetramer construct are shown below in Table 42.

TABLE 42
Results of glycoprotein fusion analysis

| | | Increased | Increased |
|---|---|---|---|
| | | fold vs BCN | fold vs. KCN |
| Glycosylation | CD45: B-Casein- | 12 | — |
| | tetramer | | |
| Glycosylation | SP tag: k-casein | — | 13 |

### Example 10: Cheese Composition Made with Beta-Casein Protein

To test whether a cheese composition having acceptable organoleptic and physical properties could be made using only beta-casein protein (i.e., without any other caseins), isolated beta casein from bovine milk was the sole casein protein used in the recipe below. The beta casein was provided in the form of a powder, comprising 84% protein with >98% purity of beta casein. An exemplary formulation for a 100% beta casein cheese is provided in Table 43.

TABLE 43
100% beta casein cheese composition

| Ingredient | Percent total |
|---|---|
| Water | 42.07% |
| Butter | 31.25% |
| Beta casein powder (84% protein) | 13.10% |
| Modified potato starch | 11.00% |
| Salt | 1.70% |
| Sodium citrate | 0.80% |
| Calcium chloride | 0.08% |

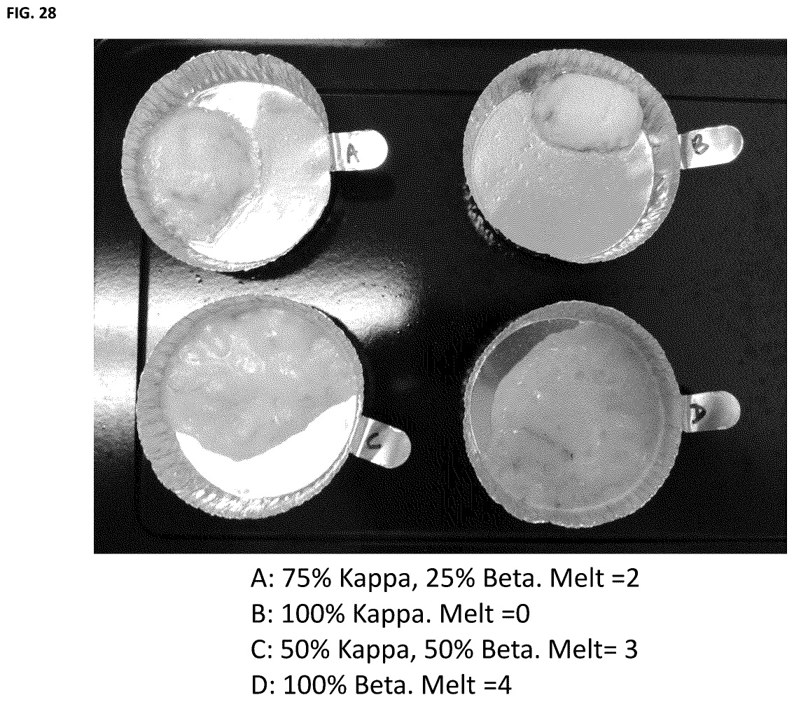

To make the cheese composition, all ingredients were added to a rapid visco analyzer (RVA) tube. The mixture was heated to 40° C. and mixed at 200 RPM for 2 minutes. The speed was increased to 500 RPM and mixed for an additional 3 minutes. The mixture was then allowed to rest for a minimum of 5 minutes before heating to 95° C. and mixing at 960 RPM for 1 minute. Then, the speed and temperature were reduced to 500 RPM and 90° C. for 1 minute. The temperature was reduced further to 85° C. and the composition was mixed for one more minute at 500 RPM. The hot cheese composition was poured into cylindrical molds (¾″ diameter pipe, 1″ in length with cap on bottom), covered with plastic wrap, and refrigerated for a minimum of 5 days. The target pH was 5.5 to 5.7, and was adjusted with lactic acid, citric acid, or sodium citrate. Meltability was analyzed as described below (see also the cheese labeled “D” in FIG. 28)

A cheese composition was also made with 50% beta casein protein and reduced amounts of other casein proteins using the following recipe shown in Table 44.

TABLE 44
50% Beta casein cheese composition

| Ingredient | Percent total |
|---|---|
| Water | 43.7% |
| Butter | 31.3% |
| Acid casein (95% protein dry basis) | 10.4% |
| Modified potato starch | 6.00% |
| Beta casein powder (84% protein) | 2.80% |
| Trisodium citrate | 1.70% |
| Salt | 1.70% |
| Sodium aluminum phosphate, basic | 1.70% |
| Citric acid | 0.70% |

To make the cheese composition, 60% of the water and all other ingredients were added to a RVA tube and heated to 50° C. and mixed at 500 RPM for 5 minutes. The remaining water was added to the RVA tube, and the mixture was heated to 95° C. and mixed at 960 RPM for 1 minute, then reduced to 500 RPM and 90° C. for 1 minute. The temperature was reduced to 85° C., and the composition was mixed at 500 RPM for one more minute. The hot cheese composition was poured into cylindrical molds (¾″ diameter pipe, 1″ in length with cap on bottom), covered with plastic wrap, and refrigerated for 5 days. The target pH was 5.5 to 5.7, and was adjusted with lactic acid, citric acid, or sodium citrate. For cheese analysis, see samples 3 and 4 of Table 49.

### Example 11: Cheese Composition Made with Kappa Casein Protein

To test whether a cheese composition having acceptable organoleptic and physical properties could be made using only kappa-casein protein (i.e., without any other casein proteins), isolated kappa casein from bovine was the sole casein protein used in the recipe below. The kappa-casein was provided in the form of a powder, which comprised 85% protein and greater than 70% purity of kappa casein. An exemplary formulation for a 100% kappa casein cheese is shown in Table 45.

TABLE 45
100% Kappa casein cheese composition

| Ingredient | Percent Total |
|---|---|
| Water | 45.0% |
| Butter | 31.3% |
| Kappa casein powder | 13.8% |
| Modified potato starch | 6.0% |
| Salt | 1.7% |
| Sodium citrate | 0.6% |
| Citric acid | 0.6% |
| Sodium aluminum phosphate (basic) | 0.9% |
| Calcium chloride | 0.1% |

All ingredients were added to a rapid visco analyzer (RVA) tube. The mixture was heated to 40° C. and mixed at 200 RPM for 2 minutes. The speed was increased to 500 RPM, and the composition was mixed for an additional 3 minutes. The mixture was then allowed to rest for a minimum of 5 minutes before heating to 95° C., and mixing at 960 RPM for 1 minute. Then, the speed and temperature were reduced to 500 RPM and 90° C. for 1 minute. The temperature was reduced further to 85° C., and the composition was mixed for one more minute at 500 RPM. The hot cheese composition was poured into cylindrical molds (¾″ diameter pipe, 1″ in length with cap on bottom), covered with plastic wrap, and refrigerated for 5 days. The target pH was 5.5 to 5.7 and was adjusted with lactic acid, citric acid, or sodium citrate. Meltability was analyzed as described below (see also the cheese labeled “B” in FIG. 28).

### Example 12: Cheese Composition Made with Alpha-Casein Protein

To test whether a cheese composition having acceptable organoleptic and physical properties could be made using only alpha-casein proteins, isolated alpha casein (a mixture of alpha-S1 and alpha-S2 caseins) from bovine was the sole casein protein used in the recipe below. The alpha-casein was provided in the form of a powder, which comprised approximately 87% protein and greater than 90% purity of alpha casein. An exemplary recipe for a 100% alpha casein cheese composition is provided below at Table 46.

TABLE 46
100% alpha casein cheese composition

| Ingredient | Percent Total |
|---|---|
| Water | 41.8% |
| Butter | 31.3% |
| Alpha casein powder | 12.6% |
| Modified potato starch | 11.0% |
| Salt | 1.7% |
| Sodium citrate | 0.8% |
| Citric acid | 0.2% |
| Sodium aluminum phosphate (basic) | 0.5% |
| Calcium chloride | 0.08% |

All ingredients were added to a rapid visco analyzer (RVA) tube. The mixture was heated to 40° C. and mixed at 200 RPM for 2 minutes. The speed was increased to 500 RPM, and the composition was mixed for an additional 3 minutes. The mixture was then allowed to rest for a minimum of 5 minutes before heating to 95° C. and mixing at 960 RPM for 1 minute. Then, the speed and temperature were reduced to 500 RPM and 90° C. for 1 minute. The temperature was reduced further to 85° C., and the composition was mixed for one more minute at 500 RPM. The hot cheese composition was poured into cylindrical molds (¾″ diameter pipe, 1″ in length with cap on bottom), covered with plastic wrap, and refrigerated for 5 days. The target pH was 5.5 to 5.7 and was adjusted with lactic acid, citric acid, or sodium citrate. Meltability was analyzed as described below.

### Example 13: Cheese Composition Made with Alpha- and Beta-Casein Proteins

To test whether a cheese composition having acceptable organoleptic and physical properties could be made using alpha- and beta-casein, alpha-casein and beta-casein powder obtained from bovine were used to create cheese compositions comprising 5000 alpha-casein and 50% beta-casein, 25% alpha-casein and 75% beta-casein, and 75% alpha-casein and 25% beta-casein, as shown in the recipes below at Table 47.

TABLE 47
Exemplary receipt for cheese composition
made with alpha- and beta-casein proteins

| | 50% | 25% | 75% |
|---|---|---|---|
| | alpha-casein | alpha-casein | alpha-casein |
| | and 50% | and 75% | and 25% |
| | beta-casein | beta-casein | beta-casein |
| Water | 42.0% | 41.8% | 42.0% |
| Butter | 31.3% | 31.3% | 31.3% |
| Modified potato starch | 10.5% | 10.5% | 10.5% |
| Alpha casein powder | 6.3% | 3.2% | 9.5% |
| Beta casein powder (84% | 6.5% | 9.8% | 3.3% |
| protein) | | | |
| Trisodium citrate | 0.9% | 0.9% | 0.9% |
| Salt | 1.7% | 1.7% | 1.7% |
| Sodium aluminum | 0.5% | 0.5% | 0.5% |
| phosphate, basic | | | |
| Citric acid | 0.2% | 0.2% | 0.2% |
| Calcium chloride | 0.08% | 0.08% | 0.08% |

To make the cheese composition, 60% of the water and all other ingredients were added to a RVA tube and heated to 50° C. The composition was then mixed at 500 RPM for 5 minutes. The remaining water was added, and the mixture was heated to 95° C. and mixed at 960 RPM for 1 minute. Then, the composition was mixed at 500 RPM at 90° C. for 1 minute. The temperature was reduced to 85° C., and the composition was mixed at 500 RPM for one more minute. The hot cheese composition was poured into cylindrical molds (¾″ diameter pipe, 1″ in length with cap on bottom), covered with plastic wrap, and refrigerated for 5 days. The target pH was 5.5 to 5.7, and was adjusted with lactic acid, citric acid, or sodium citrate. Meltability was analyzed as described below.

### Example 14: Cheese Composition Made with Beta- and Kappa-Casein Proteins

To test whether a cheese composition having acceptable organoleptic and physical properties could be made using beta- and kappa-casein, bovine kappa-casein and beta-casein powder were used to create two cheese compositions, one comprising 75% kappa casein and 25% beta casein, and another comprising 50% kappa casein and 50% beta casein, as shown in the recipes below at Table 48.

TABLE 48
Exemplary receipt for cheese composition
made with beta- and kappa-casein proteins

| | 75% kappa | 50% kappa |
|---|---|---|
| | casein and | casein and |
| | 25% beta casein | 50% beta casein |
| Water | 41.9% | 41.7% |
| Butter | 31.3% | 31.3% |
| Modified potato starch | 10.5% | 10.5% |
| Kappa casein powder | 9.7% | 6.5% |
| Beta casein powder (84% protein) | 3.3% | 6.6% |
| Trisodium citrate | 0.8% | 0.8% |
| Salt | 1.7% | 1.7% |
| Sodium aluminum phosphate, basic | 0.5% | 0.5% |
| Citric acid | 0.3% | 0.3% |
| Calcium chloride | 0.04% | 0.08 |

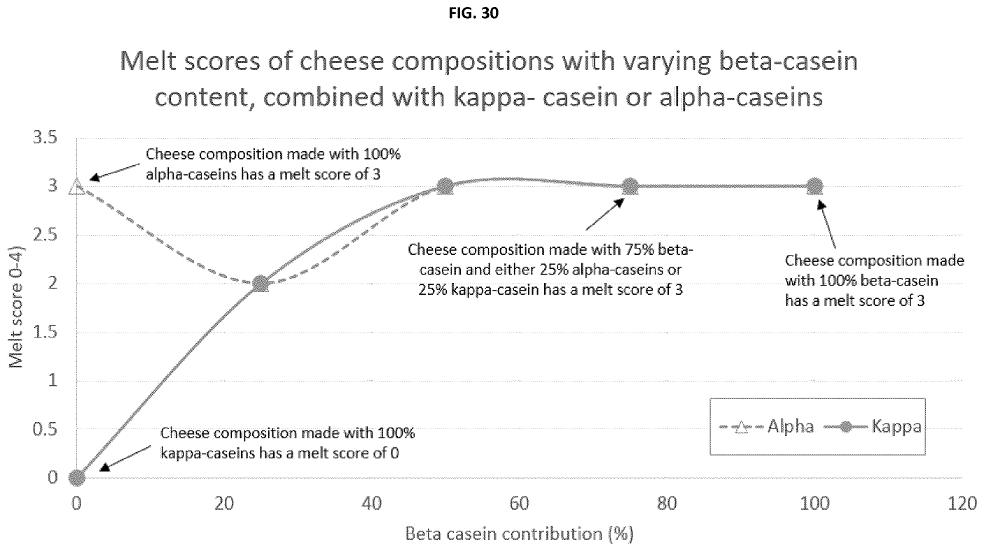

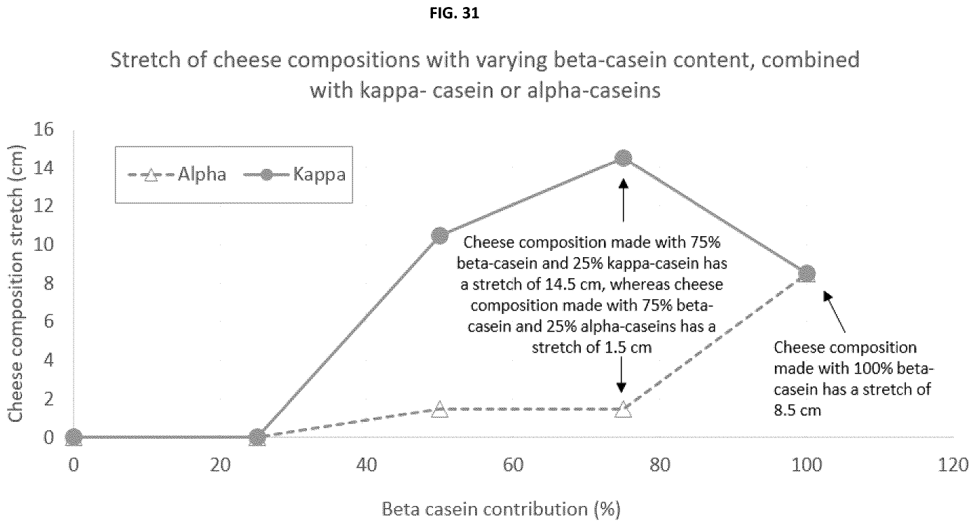

To make the cheese composition, 60% of the water and all other ingredients were added to a RVA tube and heated to 50° C. The composition was then mixed at 500 RPM for 5 minutes. The remaining water was added, and the mixture was heated to 95° C. and mixed at 960 RPM for 1 minute. Then, the composition was mixed at 500 RPM at 90° C. for 1 minute. The temperature was reduced to 85° C., and the composition was mixed at 500 RPM for one more minute. The hot cheese composition was poured into cylindrical molds (¾″ diameter pipe, 1″ in length with cap on bottom), covered with plastic wrap, and refrigerated for 5 days. The target pH was 5.5 to 5.7, and was adjusted with lactic acid, citric acid, or sodium citrate. Meltability was analyzed as described below (see also the cheeses labeled “A” and “C” in FIG. 28). Melt scores of the various cheese compositions are shown in FIG. 30 and stretch of cheese compositions is provided in FIG. 31.

### Example 15: Functional Properties of Cheese Compositions Made with Beta- and Kappa-Caseins

To test the organoleptic and physical properties, the cheese compositions were analyzed for various properties, including melt, stretch, firmness, and transparency. For the melting test, the cheeses were placed in a 450° F. oven for 5 minutes. A score of 0=no change from the initial appearance; 1=up to 25% coverage of pan; 2=25% to 50% pan coverage; 3=50% to 75% pan coverage; and 4=greater than 75% pan coverage. Shown in FIG. 28 are the results of the test with cheese compositions comprising (A) 75% kappa casein, 25% beta casein; (B) 100% kappa casein; (C) 50% kappa casein, 50% beta casein; and (D) 100% beta casein. Composition A had a melt score of 2; composition B was unchanged and therefore had a melt score of 0; composition C had a melt score of 3; and composition D exhibited the greatest meltability with a score of 4.

Additional cheese composition samples comprising different ratios of caseins and total protein were analyzed for stretchability and meltability after aging for a minimum of 5 days (Tables 49-51). Cheese composition stretch was measured by an assay testing the ability to stretch (cm in length) without breaking, after heating a 100 gram mass of the emulsion to a temperature of about 225° C. for 4 minutes and cooling to about 90° C. and pulling with a fork placed beneath the mass. Firmness and transparency were also observed by sensory evaluation (data not shown).

Shown in Table 49, below, are data collected from cheese compositions comprising between 11-11.5% protein, and in Table 50, data collected from cheese comprising between 13-13.500 protein. Compositions 1 and 2 comprise caseins at a ratio that is similar to the approximate percentages of caseins in bovine milk. Compositions 3, 4, 7, and 8 have levels of beta-casein higher than that found in milk, and compositions 5, 6, 8, 9, and 10 have levels of kappa-casein higher than that found in bovine milk.

TABLE 49
Stretch and melt of cheese compositions
comprising between 11-11.5% protein

| | Protein contribution (%) | | | | | |
|---|---|---|---|---|---|---|
| | Alpha-S1 + | | | | Stretch | CaCl₂ |
| | Alpha-S2 | Beta | Kappa | Melt | (cm) | (% w/w) |
| 1 | 50 | 37.5 | 12.5 | 4 | 5 | 0 |
| 2 | 50 | 37.5 | 12.5 | 4 | 8.5 | 0.25 |
| 3 | 40 | 50 | 10 | 3 | 5 | 0 |
| 4 | 40 | 50 | 10 | 3 | 8.5 | 0.25 |
| 5 | 43 | 32 | 25 | 4 | 6.5 | 0 |
| 6 | 43 | 32 | 25 | 3 | 3 | 0.25 |
| 7 | 0 | 100 | 0 | 4 | 20 | 0.4 |
| 8 | 0 | 50 | 50 | 3 | 17.5 | 0.08 |
| 9 | 0 | 25 | 75 | 2 | 0 | 0.04 |
| 10 | 0 | 0 | 100 | 0 | 0 | 0.12 |

TABLE 50
Stretch and melt of cheese compositions
comprising between 13-13.5% protein

| | Protein contribution (%) | | | | | |
|---|---|---|---|---|---|---|
| | Alpha-S1 + | | | | Stretch | CaCl₂ |
| | Alpha-S2 | Beta | Kappa | Melt | (cm) | (% w/w) |
| 1 | 50 | 37.5 | 12.5 | 3 | 5 | 0 |
| 2 | 50 | 37.5 | 12.5 | 2 | 5.5 | 0.25 |
| 3 | 34 | 57 | 9 | 2.5 | 5 | 0 |
| 4 | 34 | 57 | 9 | 3 | 5 | 0.25 |
| 5 | 39 | 32 | 29 | 3 | 4.5 | 0 |
| 6 | 39 | 32 | 29 | 2 | 4 | 0.25 |

As evidenced by the data above, cheese compositions made with beta-casein exhibited good melting and stretch after aging for at least five days. Use of the tested amounts of beta-casein also softened the cheese composition compared to standard casein ratios. The beta-casein cheese composition was affected by calcium level similar to that of the control cheese composition, and it was also found to be highly soluble. The cheese composition was substantially transparent when melted.

Kappa-casein imparted firmness to the cheese composition relative to standard casein ratios, but was more reactive to calcium than beta casein and control cheese compositions 1 and 2. Levels of less than 25% kappa casein did not impact stretch and stretch may improve slightly, whereas increasing levels of kappa casein restricted melt and reduced stretch after refrigeration for five days. Immediately after cooking, the 100% kappa casein cheese compositions can stretch to greater than 25 cm.

The alpha-caseins, alpha-S1-casein and alpha-S2-casein, are assumed to provide cheese firmness, as cheeses with depleted levels of alpha-S1-casein and alpha-S2-casein are softer than the control cheeses.

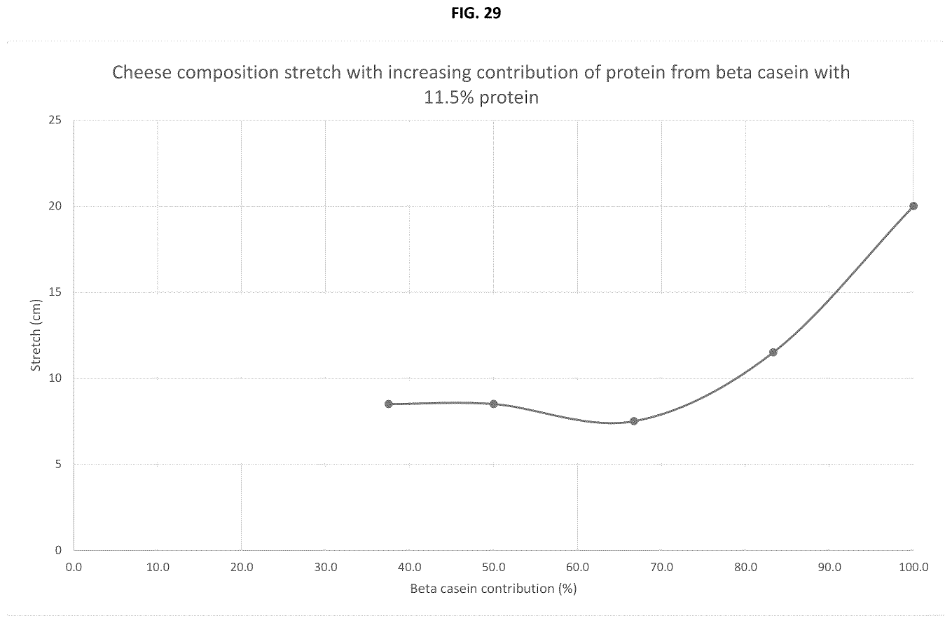

Cheese stretch was impacted by percent contribution of beta-casein. Specifically, increasing the amount of beta-casein correlated with an increase in stretch. As shown below, in Table 51 and FIG. 29, cheese compositions comprising, for example, 50% beta-casein had a stretch of 8.5 cm, whereas cheese comprising 100% beta-casein had a stretch of 20 cm. All cheese compositions comprised 11.5% total protein and CaCl₂).

TABLE 51
Cheese stretch with increasing contribution
of protein from beta-casein

| % beta-casein | Stretch (cm) |
|---|---|
| 37.5 | 8.5 |
| 50.0 | 8.5 |
| 66.7 | 7.5 |
| 83.3 | 11.5 |
| 100.0 | 20 |

### Example 16: Functional Properties of Additional Cheese Compositions

The analysis performed in Example 15 was repeated with additional compositions, as shown in Tables 52-54. The compositions comprised 100% beta-casein, 100% kappa-casein, or 10000 alpha-caseins, or mixtures thereof. The actual percent of protein by weight of the compositions varied between 10-11.75%, and the CaCl₂ concentration ranged from 000 to 0.16% (by weight). Melt, stretch, and firmness were determined as described above.

TABLE 52
Functional properties of additional cheese
compositions comprising only one casein

| | | | | | |
|---|---|---|---|---|---|
| % protein | % CaCl₂ | melt | stretch | firmness | pH |

100% Beta-casein

| | | | | | |
|---|---|---|---|---|---|
| 11.75 | 0 | 2 | 1.5 | very firm | 5.23 |
| 10 | 0 | 3 | 15 | soft | 5.28 |
| 10 | 0 | 3 | 0 | very soft | 5.6 |
| 11 | 0.04 | 4 | 6.5 | firm | 5.63 |
| 11 | 0.08 | 4 | 20 | firm | 5.55 |
| 11 | 0.08 | 3 | 6.5 | soft | 5.59 |

100% Kappa-casein

| | | | | | |
|---|---|---|---|---|---|
| 11.75 | 0.2 | 2 | 0 | firm | 5.65 |
| 11.75 | 0.12 | 3 | 0 | firm | 5.44 |
| 11.75 | 0.06 | 2 | 1.5 | firm | 5.67 |
| 11 | 0.04 | 2 | 0 | firm | 5.67 |
| 11.75 | 0.12 | 1 | 0 | firm | 5.06 |

100% Alpha-casein

| | | | | | |
|---|---|---|---|---|---|
| 11* | 0 | 3 | 0 | brittle | 6.01 |
| 11* | 0.08 | 3 | 0 | slightly soft | 5.8 |
| 11* | 0 | 3 | 0 | firm | 5.91 |
| 11* | 0.08 | 3 | 0 | firm | 5.9 |
| 11 | 0 | 2 | 0 | very firm | 6.26 |

*indicates that the compositions were undercooked

TABLE 53
Functional properties of additional cheese
compositions comprising mixtures of caseins
Beta-casein and Kappa-casein

| Sample | | | | | | | |
|---|---|---|---|---|---|---|---|
| No. | % beta | % kappa | % CaCl₂ | melt | stretch | firmness | pH |
| 1 | 5.5 | 5.5 | 0.04 | 4 | 7 | firm | 5.5 |
| 2 | 2.75 | 8.25 | 0.04 | 2 | 0 | very firm | 5.64 |
| 3 | 5.5 | 5.5 | 0.08 | 4 | 17.5 | firm | 5.36 |

Alpha-caseins and beta-casein

| | % alpha | % beta | % CaCl₂ | melt | stretch | firmness | pH |
|---|---|---|---|---|---|---|---|
| 1 | 5.5 | 5.5 | 0.16 | 4 | 0 | firm | 5.87 |
| 2 | 5.5 | 5.5 | 0.08 | 3 | 1.5 | firm | 6.01 |
| 3 | 5.5 | 5.5 | 0 | 3 | 1.5 | firm | 5.98 |
| 4 | 2.75 | 8.25 | 0.16 | 4 | 5 | firm | 5.94 |
| 5 | 2.75 | 8.25 | 0.08 | 3 | 1.5 | firm | 5.72 |
| 6 | 2.75 | 8.25 | 0 | 4 | 5.5 | firm | 5.68 |
| 7 | 8.25 | 2.75 | 0.16 | 3 | 0 | firm | 5.91 |
| 8 | 8.25 | 2.75 | 0.08 | 2 | 0 | firm | 5.92 |
| 9 | 8.25 | 2.75 | 0 | 2 | 0 | firm | 5.91 |

TABLE 54
Cheese melt and stretch with increasing
contribution of protein from beta-casein

| % Beta- | % Alpha- | % Kappa- | | Stretch |
|---|---|---|---|---|
| casein | caseins | casein | Melt | (cm) |
| 0 | 100 | 0 | 3 | 0 |
| 0 | 0 | 100 | 0 | 0 |
| 25 | 75 | 0 | 2 | 0 |
| 25 | 0 | 75 | 2 | 0 |
| 50 | 50 | 0 | 3 | 1.5 |
| 50 | 0 | 50 | 3 | 10.5 |
| 75 | 25 | 0 | 3 | 1.5 |
| 75 | 0 | 25 | 3 | 14.5 |
| 100 | 0 | 0 | 3 | 8.5 |

As shown by the data in Tables 52-54 above and FIGS. 30-31, beta-casein is the only casein that imparts both melt and stretch in a cheese composition under these conditions. Alpha caseins do not appear to impart stretch and do not significantly restrict melt. Alpha-caseins contribute to cheese firmness (data not shown). Kappa-casein also imparts firmness but negatively impacts melt. Like beta-casein it can contribute to stretch but only when combined with another protein.

### Example 17: Cheese Compositions with Beta-Lactoglobulin

To determine the effect of adding beta-lactoglobulin on the functional and organoleptic properties of the compositions, various cheese compositions were generated with different amounts of beta-lactoglobulin.

TABLE 55
Stretch and melt of cheese composition with the addition of beta-lactoglobulin
Protein contribution (%)

| Alpha- | | | | | | | |
|---|---|---|---|---|---|---|---|
| S1 + | | | | | | | |
| Alpha- | | | Beta | | % | | Stretch |
| S2 | Beta | Kappa | Lactoglobulin | Soy | Protein | Melt | (cm) |
| 50 | 37.5 | 12.5 | 0 | 0 | 11.5 | 4 | 5 |
| 39.8 | 29.8 | 9.9 | 20.5 | 0 | 11.5 | 3 | 0 |
| 39.8 | 29.8 | 9.9 | 0 | 20.5 | 11.5 | 4 | 4.5 |
| 50 | 37.5 | 12.5 | 0 | 0 | 13.2 | 3 | 8.5 |
| 34.6 | 26 | 8.7 | 30.8 | 0 | 13.2 | 2 | 0 |
| 34.6 | 26 | 8.7 | 0 | 30.8 | 13.2 | 3 | 0 |

Gram of protein/100 g of cheese

| | | | | | Total | | |
|---|---|---|---|---|---|---|---|
| AS1 + | | | | | Protein | | Stretch |
| AS2 | Beta | Kappa | BLG | Soy | (g.) | Melt | (cm) |
| 5.75 | 4.31 | 1.44 | 0.00 | 0.00 | 11.5 | 4 | 5 |
| 4.58 | 3.43 | 1.14 | 2.36 | 0.00 | 11.5 | 3 | 0 |
| 4.58 | 3.43 | 1.14 | 0.00 | 2.36 | 11.5 | 4 | 4.5 |
| 6.60 | 4.95 | 1.65 | 0.00 | 0.00 | 13.2 | 3 | 8.5 |
| 4.57 | 3.43 | 1.15 | 4.07 | 0.00 | 13.2 | 2 | 0 |
| 4.57 | 3.43 | 1.15 | 0.00 | 4.07 | 13.2 | 3 | 0 |

As shown in Table 55 above, at 20% and 3000 casein replacement levels, stretch was eliminated. Cheese composition melt, stretch, firmness were closer to the control with soy protein isolate compared to beta-lactoglobulin at both replacement levels tested.

Similar results were achieved with compositions comprising kappa-casein and beta-lactoglobulin (Table 56), beta-casein and beta-lactoglobulin (Table 57). The addition of beta-lactoglobulin to the cheese compositions softened them, restricted melt, and imparted an opacity due to protein aggregation.

TABLE 56
Stretch and melt of kappa-casein cheese composition
with the addition of beta-lactoglobulin
Kappa-casein + Beta-lactoglobulin

| % protein | % CaCl₂ | melt | stretch | firmness | pH | % BLG |
|---|---|---|---|---|---|---|
| 10 | 0.06 | 2 | 0 | firm | 5.95 | 2.5 |

TABLE 57
Stretch and melt of beta-casein cheese composition
with the addition of beta-lactoglobulin
Beta-casein + beta-lactoglobulin

| % protein | % CaCl₂ | melt | stretch | firmness | pH | % BLG |
|---|---|---|---|---|---|---|
| 10 | 0 | 3 | 0 | slightly | 5.43 | 2.5 |
| | | | | soft | | |
| | | | | (between | | |
| | | | | soft and | | |
| | | | | firm) | | |

### Example 18: Estimation of Apparent Viscosity

An exemplary milk composition comprising beta-casein as the only casein (BC milk), a yogurt composition comprising beta-casein as the only casein (BC yogurt), and an ice cream mix composition comprising beta-casein as the only casein (BC IC mix) are described in Table 58, below.

TABLE 58
Food compositions for viscosity analysis

| Ingredient | % |
|---|---|
| Beta casein milk | |
| Beta casein powder | 3.0 |
| Sodium citrate | 0.2 |
| Lactic acid (88%) | 0.1 |
| Sodium chloride | 0.2 |
| Calcium chloride | 0.2 |
| Palm oil | 3.3 |
| Soy lecithin | 0.2 |
| Glucose | 4.0 |
| Water | 88.9 |
| Beta casein yogurt (plain) | |
| Beta casein powder | 4.0 |
| Sodium citrate | 0.2 |
| Sodium chloride | 0.2 |
| Calcium chloride | 0.2 |
| Coconut oil | 4.0 |
| Soy lecithin | 0.3 |
| Modified tapioca starch | 3.5 |
| Glucose | 4.0 |
| Water | 83.6 |
| Beta casein ice cream mix | |
| Beta casein powder | 4.5 |
| Sodium citrate | 0.2 |
| Lactic acid (88%) | 0.1 |
| Sodium chloride | 0.2 |
| Tetrasodium pyrophosphate | 0.2 |
| Cocoa butter | 12.0 |
| Calcium sulfate | 0.2 |
| Mono & diglycerides | 0.5 |
| Cellulose gum | 0.2 |
| Sucrose | 20.0 |
| Vanilla extract | 0.5 |
| Water | 61.5 |

To make a BC milk composition, water is heated to 100-120° F., and lecithin and melted palm oil are added. Subsequently, the remaining ingredients (except lactic acid) are added with agitation and minimal air incorporation. The pH is adjusted to the range of 6.5-7.0 with lactic acid. The composition is then heated to 140° F. and homogenized (two stage, 2000 psi total). Then, the composition is heated to 175° F. and held at that temperature for 20 seconds before cooling to 40° F.

To make a BC yogurt composition, water is heated to 100-120° F., and lecithin and melted coconut oil are added. Subsequently, the remaining ingredients (except lactic acid) are added with agitation and minimal air incorporation. The pH is adjusted to the range of 6.5-7.0 with lactic acid. The composition is then heated to 140° F. and homogenized (two stage, 2000 psi total). Then, the composition is heated to 185° F. and held at that temperature for 5 minutes. The composition is then cooled to 110° F. and yogurt cultures are added. The composition is fermented at 108° F. until the pH is 4.6. The composition is then stirred and cooled to 40° F.

To make a BC IC composition, water is heated to 100-120° F., and lecithin and melted cocoa butter are added. Subsequently, the remaining ingredients (except lactic acid) are added with agitation and minimal air incorporation. The pH is adjusted to the range of 6.5-7.0 with lactic acid. The composition is then heated to 140° F. and homogenized (two stage, 2000 psi total). Then, the composition is heated to 175° F. and held at that temperature for 20 seconds before cooling to 40° F.

Theoretical approximations of the apparent viscosity for these BC milk, BC yogurt, and BC IC mix may be determined. Specifically, these approximations may be based on the rheological analysis of formulations made with bovine milk and adjustments made from observations made during the work with various cheese compositions described herein.

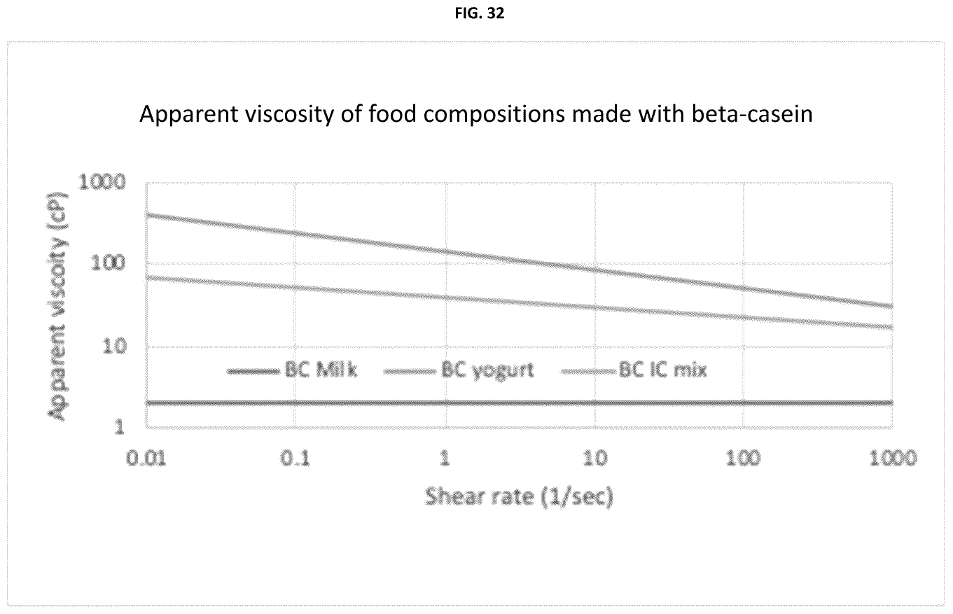

Approximations are shown in FIG. 32. The BC milk compositions are estimated to have an apparent viscosity of about 2 cP over all shear rates analyzed. In contrast, the BC yogurt and the BC IC mix compositions are estimated to have a higher apparent viscosity, which is expected to decrease at higher shear rates, characteristic of non-Newtonian compositions.

Taken together, this data indicates that the BC yogurt and BC IC mix compositions are expected to be non-Newtonian compositions.

### Example 19: Expression of a Fusion Protein Comprising Beta-Casein in E. Coli

To determine whether the fusion proteins of the disclosure may be detectably expressed in a bacterial system, a beta-casein tetramer (i.e., a fusion protein comprising four beta-caseins) was expressed in E. Coli. Specifically, the pET system (Novagen) was used for the cloning and expression of the proteins of interest in E. coli. A DNA sequence encoding the beta-casein tetramer was PCR amplified and cloned into the NcoI and BlpI sites of pET-28a (+) expression vector via In-Fusion (Takara) cloning. The ligated vector was transformed into Stellar™ competent cells. Subsequently, the DNA of positive clones was used to transform BL21-CodonPlus strain (Agilent Technologies) which encodes a T7 RNA polymerase under control of the lacUV5 promoter for easy expression.

To induce protein expression, an overnight culture grown to stationary phase was diluted (1/100) and then grown to mid-log phase (OD600-0.4-0.6). The mid-log phase culture was pelleted, and the supernatant was removed. Protein expression was induced by the addition of IPTG (0.5 mM final concentration) to the pellet, and the cells were incubated for 3 hours at 37° C. with 160 rpm shaking. To extract the proteins of interest, the BugBuster® (Novagen) master mix was utilized following the manufacturer's instructions with the addition HALT protease inhibitor. The proteins were separated using SDS-PAGE and transferred to nitrocellulose membrane. The fusion protein was detected using a primary antibody raised against beta-casein.

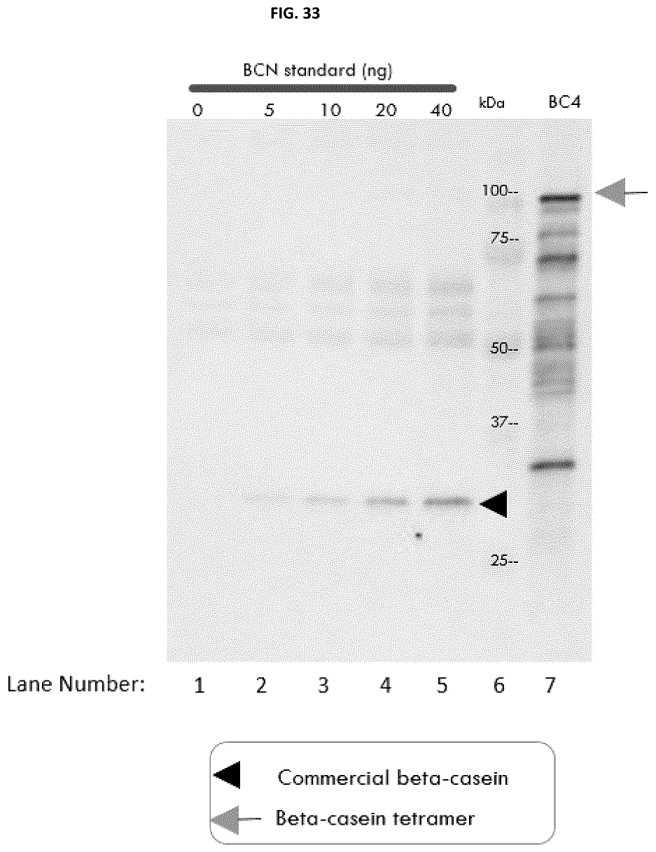

As shown in FIG. 33, the beta-casein tetramer (BC4) accumulated to detectable levels in E. Coli. Lanes 1-5 of FIG. 33 show wildtype E. coli extracts with commercial beta-casein protein spiked in at 0, 5, 10, 20 and 40 ng per lane, as a standard. Lane 6 shows molecular weight markers. Lane 7 shows Beta-casein tetramer expressed in E. Coli after 3 h of induction with IPTG.

### Example 20: Expression of a Fusion Protein Comprising Beta-Casein in Tobacco Leaves

To determine whether the fusion proteins of the disclosure may be detectably expressed in a tobacco system, a fusion protein comprising beta-casein fused to beta-lactoglobulin was expressed in tobacco leaves.

A DNA sequence encoding a fusion protein comprising, from N-terminus to C-terminus, beta-lactoglobulin and beta-casein, was inserted into the AR17 vector backbone in between a double 35S promoter and EUT:Rb7T double terminator. The plasmid was transformed into agrobacterium strain AGL1 and the positive agrobacterium colonies were cultured overnight in selective media. To prepare the infiltration solution, the agrobacterium culture was precipitated by centrifugation at 1,000 g for 10 mins and resuspended in equal volume of the infiltration medium (50 mM MES, 2 mM Na₃PO₄, 5 mg/mL D-glucose and 0.1 mM acetosyringone). This washing step was repeated a second time.

The fusion protein expressing strain was co-infiltrated in tobacco leaves with the post-translational gene silencing inhibitor p19 strain and the protease inhibitor NbPR4 strain to enhance the fusion protein expression. Concentration of the fusion protein expressing-strain, and strains p19 and NbPR4, was adjusted to an optical density (OD) of 1, 0.5 and 0.5 respectively, immediately before co-infiltration into the leaves. Six to eight-week-old Nicothiana benthamiana plants were used for infiltrations. Four different fully expanded leaves were infiltrated as biological replicates.

Protein samples were harvested three days after infiltration. Total soluble proteins were extracted with equal volume of extraction buffer (1×PBS PH7.4, 5 mM DTT, 0.1% Tween20 and 1×HALT protease inhibitors). Total protein concentrations were measured using Pierce 660 reagent. To visualize the target protein expression, 1 ug of total soluble protein were separated on SDS-PAGE, transferred to nitrocellulose membrane, and probed with a beta-casein primary antibody.

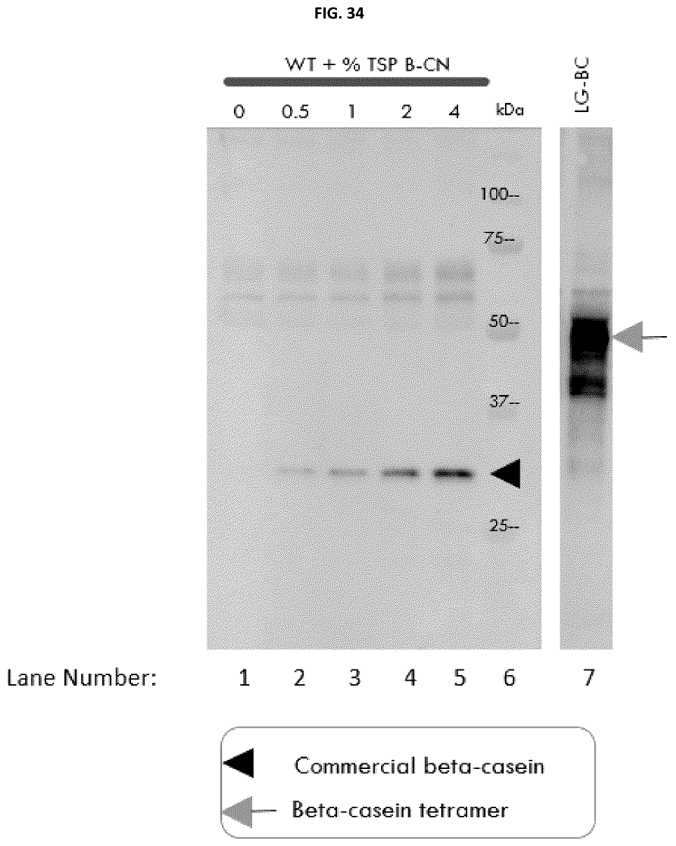

Results are shown in FIG. 34. Lanes 1-5 show wild type tobacco protein extracts spiked with 0, 0.5, 1, 2, or 4 nanograms of commercially available beta-casein. Lane 6 shows molecular weight markers. Lanes 7-8 shows infiltration of tobacco leaves with the fusion protein. This data shows that the fusion protein accumulated in tobacco leaves at a level above 4% total soluble protein.

### Example 21: Protease Knock Out or Expression of Protease Inhibitors in Plants to Reduce or Eliminate Degradation of Transgenically Expressed Proteins

The prediction software PROSPER was used to elucidate families of proteases that might be responsible for the degradation of transgenically expressed proteins and to generate a list of proteases and protease inhibitors for use in the generation of knock out lines or for the co-expression of a protease inhibitor with a protein to be transgenically expressed, for example as in FIG. 11B. The proteases cysteine, aspartic, and serine were identified as being highly expressed in soybean seeds.

### Generation of Protease Knock Out Lines

Transcriptome and proteomic data was utilized to identify highly expressed serine, cysteine and aspartic proteases for use in protease knock-out lines or to generate constructs to inhibit them. The protease inhibitors are shown in Table 59.

TABLE 59
Exemplary protease inhibitors

| | Nucleic Acid | Amino Acid |
|---|---|---|
| Protease | SEQ ID NO | SEQ ID NO |
| SICYS8 | 945 | 946 |
| CID | 947 | 948 |
| BB | 949 | 950 |
| API5 | 951 | 952 |
| KTi3 | 953 | 954 |

Co-delivery of protease inhibitors with a transgenic protein

As protease activity is pH dependent, some families of proteases reside in different cellular compartments. For example, aspartic protease families are abundant in the endoplasmic reticulum (ER), thus, degradation of transgenically expressed proteins can be reduced by co-expression of an aspartic protease inhibitor with the transgenic protein. Transgenically expressed proteins can be targeted to the ER by way of a KDEL addition. Therefore, constructs were generated to co-express a protease inhibitor and a transgenic milk protein. In summary, proteases were selected based on their expression level, expression pattern during seed development, and the family of proteases that they belong to with the focus on aspartic, cysteine and serine proteases. In the case of the protease inhibitors, inhibitors that target the most highly expressed proteases were selected. Table 60 below shows exemplary proteases and example inhibitors.

TABLE 60
Exemplary proteases and inhibitors of the same

| | | | | | DNA | Protein |
|---|---|---|---|---|---|---|
| | | | | | Sequence | Sequence |
| | | | | | SEQ ID | SEQ ID |
| | Identification | Gene ID | Type | Strategy | NO | NO |
| Protease 1 | Peptidase A1 | Glyma.04g091800 | Aspartic-type | Knock-out | 917 | 918 |
| | domain- | | protease | | | |
| | containing | | | | | |
| | protein | | | | | |
| Protease 2 | Peptidase A1 | Glyma.02g213000 | Aspartic-type | Not | 919 | 920 |
| | domain- | | protease | selected | | |
| | containing | | | | | |
| | protein | | | | | |
| Protease 3 | Cysteine | Glyma.10g207100 | Cysteine | Knock-out | 921 | 922 |
| | proteinase | | protease | | | |
| Protease 4 | 34 kDa | Glyma.08g116300 | Cysteine | Knock-out | 923 | 924 |
| | maturing seed | | protease | | | |
| | protein | | | | | |
| Protease 3 | Cysteine | | Cysteine | Knock-out | 925 | 926 |
| and 4 | proteinase/34 | | protease | | | |
| (Simultaneous | kDa maturing | | | | | |
| KO) | seed protein | | | | | |
| Protease 5 | Uncharacterized | Glyma.06g275300 | Cysteine | Not | 927 | 928 |
| | protein | | protease | selected | | |
| Protease 7 | Carboxypeptidase | Glyma.09g226700 | Serine-type | Not | 929 | 930 |
| | | | protease | selected | | |
| Protease 8 | Carboxypeptidase | Glyma.03g125400 | Serine-type | Not | 931 | 932 |
| | | | protease | selected | | |
| Protease 9 | Serine-Type | Glyma.17g164100 | Serine-type | Knock-out | 933 | 934 |
| | protease | | protease | | | |
| SICYS8 | Cystatine | Glyma08g45530 | Cysteine | Co- | 935 | 936 |
| | | | protease | expression | | |
| | | | inhibitor | | | |
| CID | Cathepsin D | X73986 | Aspartic | Co- | 937 | 938 |
| | inhibitor | | protease | expression | | |
| | | | inhibitor | | | |
| BB | Bowman-Birk | Glyma16g33400 | Trypsin- | Co- | 939 | 940 |
| | inhibitor D-II | | chymotrypsin | expression | | |
| | | | inhibitor | | | |
| API5 | Aspartic | MH686153.1 | Aspartic | Co- | 941 | 942 |
| | Protease | | Protease | expression | | |
| | Inhibitor 5 | | Inhibitor | | | |
| KTi3 | Kunitz-Trypsin | Glyma08g45530 | Trypsin | Co- | 943 | 944 |
| | inhibitor | | inhibitor | expression | | |

Constructs for protease knock out or for co-expression with protease inhibitors were designed, see Table 61.

TABLE 61
Exemplary designs for protease knock-out or co-expression

| | | Protein(s) |
|---|---|---|
| Plasmid ID | Cassette detail | abbreviation |
| AR14-07 | Cas9(KO Protease1)(GmSeed2:sig2:OBC-T3:OBC-T2:OKC1-T:OLG1:AtHSP T:AtUbi10T) | BC-BC-KCN-LG |
| AR14-08 | Cas9(KO Protease3)(GmSeed2:sig2:OBC-T3:OBC-T2:OKC1-T:OLG1:AtHSP T:AtUbi10T) | BC-BC-KCN-LG |
| AR14-09 | Cas9(KO Protease4)(GmSeed2:sig2:OBC-T3:OBC-T2:OKC1-T:OLG1:AtHSP T:AtUbi10T) | BC-BC-KCN-LG |
| AR14-10 | Cas9(KO Protease5)(GmSeed2:sig2:OBC-T3:OBC-T2:OKC1-T:OLG1:AtHSP T:AtUbi10T) | BC-BC-KCN-LG |
| AR14-11 | Cas9(KO Protease9)(GmSeed2:sig2:OBC-T3:OBC-T2:OKC1-T:OLG1:AtHSP T:AtUbi10T) | BC-BC-KCN-LG |
| AR14-12 | Cas9(KO Protease3&4)(GmSeed2:sig2:OBC-T3:OBC-T2:OKC1-T:OLG1:AtHSP T:AtUbi10T) | BC-BC-KCN-LG |
| AR14-14 | Cas9(KO Protease1)(GmSeed2:sig2:OBC-T5:OBC-T4:OBC-T3:OBC-T2:AtHSP T:AtUbi10T) | BC-BC-BC-BC |
| AR14-18 | Cas9(KO Protease9)(GmSeed2:sig2:OBC-T5:OBC-T4:OBC-T3:OBC-T2:AtHSP T:AtUbi10T) | BC-BC-BC-BC |
| AR14-19 | Cas9(KO Protease3&4) - (GmSeed2:sig2:OBC-T5:OBC-T4:OBC-T3:OBC-T2:AtHSP T:AtUbi10T) | BC-BC-BC-BC |
| AR14-14 | Cas9(KO Protease1)(GmSeed2:sig2:OBC-T5:OBC-T4:OBC-T3:OBC-T2:AtHSP T:AtUbi10T) | BC-BC-BC-BC |
| AR14-15 | Cas9(KO Protease3)(GmSeed2:sig2:OBC-T5:OBC-T4:OBC-T3:OBC-T2:AtHSP T:AtUbi10T) | BC-BC-BC-BC |
| AR14-16 | Cas9(KO Protease4)(GmSeed2:sig2:OBC-T5:OBC-T4:OBC-T3:OBC-T2:AtHSP T:AtUbi10T) | BC-BC-BC-BC |
| AR14-17 | Cas9(KO Protease5)(GmSeed2:sig2:OBC-T5:OBC-T4:OBC-T3:OBC-T2:AtHSP T:AtUbi10T) | BC-BC-BC-BC |
| AR14-18 | Cas9(KO Protease9)(GmSeed2:sig2:OBC-T5:OBC-T4:OBC-T3:OBC-T2:AtHSP T:AtUbi10T) | BC-BC-BC-BC |
| AR14-19 | Cas9(KO Protease3&4) - (GmSeed2:sig2:OBC-T5:OBC-T4:OBC-T3:OBC-T2:AtHSP T:AtUbi10T) | BC-BC-BC-BC |
| AR15-340 | (GmSeed2:sig2:OBC-T5:OBC-T4:OBC-T3:OBC-T2:AtHSP T:AtUbi10T) | BC-BC-BC-BC/CYS |
| | (PvPhas:arcUTR:sig10:SICYS8:arcT) | |
| AR15-341 | (GmSeed2:sig2:OBC-T5:OBC-T4:OBC-T3:OBC-T2:AtHSP T:AtUbi10T) | BC-BC-BC-BC/CYS |
| | (GmCEP1:sig12:SICYS8:arcT) | |
| AR15-342 | (GmSeed2:sig2:OBC-T5:OBC-T4:OBC-T3:OBC-T2:AtHSP T:AtUbi10T) | BC-BC-BC-BC/CYS |
| | (GmGRD:sig12:SICYS8:arcT) | |
| AR15-343 | (GmSeed2:sig2:OBC-T3:OBC-T2:OKC1-T:OLG1:AtHSP T:AtUbi10T) | BC-BC-KCN-LG/CYS |
| | (PvPhas:arcUTR:sig10:SICYS8:arcT) | |
| AR15-344 | (GmSeed2:sig2:OBC-T3:OBC-T2:OKC1-T:OLG1:AtHSP T:AtUbi10T) | BC-BC-KCN-LG/CYS |
| | (GmCEP1:sig12:SICYS8:arcT) | |
| AR15-345 | (GmSeed2:sig2:OBC-T3:OBC-T2:OKC1-T:OLG1:AtHSP T:AtUbi10T) | BC-BC-KCN-LG/CYS |
| | (GmGRD:sig12:SICYS8:arcT) | |
| AR15-347 | (GmSeed2:sig2:OBC-T4:OBC-T3:OBC-T2:OKC1-T:OLG1:AtHSP T:AtUbi10T) | BC-BC-BC-KCN- |
| | (PvPhas:arcUTR:sig10:SICYS8:arcT) | LG/CYS |
| AR15-348 | (GmSeed2:sig2:OBC-T4:OBC-T3:OBC-T2:OKC1-T:OLG1:AtHSP T:AtUbi10T) | BC-BC-BC-KCN- |
| | (GmCEP1:sig12:SICYS8:arcT) | LG/CYS |
| AR15-349 | (GmSeed2:sig2:OBC-T4:OBC-T3:OBC-T2:OKC1-T:OLG1:AtHSP T:AtUbi10T) | BC-BC-BC-KCN- |
| | (GmGRD:sig12:SICYS8:arcT) | LG/CYS |
| AR15-379 | (GmSeed2:sig2:OBC-T5:OBC-T4:OBC-T3:OBC-T2:AtHSP T:AtUbi10T) | BC-BC-BC-BC/CYS |
| | (GmTHIC:sig12:SICYS8:arcT) | |
| AR15-380 | (GmSeed2:sig2:OBC-T3:OBC-T2:OKC1-T:OLG1:AtHSP T:AtUbi10T) | BC-BC-KCN-LG/CYS |
| | (GmTHIC:sig12:SICYS8:arcT) | |
| AR15-381 | (GmSeed2:sig2:OBC-T4:OBC-T3:OBC-T2:OKC1-T:OLG1:AtHSP T:AtUbi10T) | BC-BC-BC-KCN- |
| | (GmTHIC:sig12:SICYS8:arcT) | LG/CYS |
| AR15-462 | (GmSeed2:sig2:OBC-T5:OBC-T4:OBC-T3:OBC-T2:AtHSP T:AtUbi10T) | BC-BC-BC-BC/CID |
| | (PvPhas:arcUTR:sig10:SICID:arcT) | |
| AR15-463 | (GmSeed2:sig2:OBC-T3:OBC-T2:OKC1-T:OLG1:AtHSP T:AtUbi10T) | BC-BC-KCN-LG/CID |
| | (PvPhas:arcUTR:sig10:SICID:arcT) | |
| AR15-464 | (GmSeed2:sig2:OBC-T5:OBC-T4:OBC-T3:OBC-T2:AtHSP T:AtUbi10T) | BC-BC-BC-BC/BB |
| | (PvPhas:arcUTR:sig10:GmBBID-II.1:arcT) | |
| AR15-465 | (GmSeed2:sig2:OBC-T3:OBC-T2:OKC1-T:OLG1:AtHSP T:AtUbi10T) | BC-BC-KCN-LG/BB |
| | (PvPhas:arcUTR:sig10:GmBBID-II.1:arcT) | |
| AR15-466 | (GmSeed2:sig2:OBC-T5:OBC-T4:OBC-T3:OBC-T2:AtHSP T:AtUbi10T) | BC-BC-BC-BC/API5 |
| | (PvPhas:arcUTR:sig10:StAPI5:arcT) | |
| AR15-467 | (GmSeed2:sig2:OBC-T3:OBC-T2:OKC1-T:OLG1:AtHSP T:AtUbi10T) | BC-BC-KCN- |
| | (PvPhas:arcUTR:sig10:StAPI5:arcT) | LG/API5 |
| AR15-468 | (GmSeed2:sig2:OBC-T5:OBC-T4:OBC-T3:OBC-T2:KDEL:AtHSP T:AtUbi10T) | BC-BC-BC-BC/API5 |
| | (PvPhas:arcUTR:sig10:StAPI5:KDEL:arcT) | |
| AR15-469 | (GmSeed2:sig2:OBC-T3:OBC-T2:OKC1-T:OLG1:KDEL:AtHSP T:AtUbi10T) | BC-BC-KCN- |
| | (PvPhas:arcUTR:sig10:StAPI5:KDEL:arcT) | LG/API5 |
| AR15-470 | (GmSeed2:sig2:OBC-T5:OBC-T4:OBC-T3:OBC-T2:KDEL:AtHSP T:AtUbi10T) | BC-BC-BC-BC/CID |
| | (PvPhas:arcUTR:sig10:SICID:KDEL:arcT) | |
| AR15-471 | (GmSeed2:sig2:OBC-T3:OBC-T2:OKC1-T:OLG1:KDEL:AtHSP T:AtUbi10T) | BC-BC-KCN-LG/CID |
| | (PvPhas:arcUTR:sig10:SICID:KDEL:arcT) | |

Exemplary constructs from Table 61 above encoding for BC-BC-BC-BC; or BC-BC-KCN-LG configured to co-express with a protease inhibitor or CRJSPR system for protease knock out were employed and the percent accumulation of the respective recombinant milk protein was determined. Table 62 and Table 63 show fold expression as compared to control beta casein. The data sets are from constructs which were expressed in the same cultivar (SA14) and selected specifically for TO plants with one or two gene copies of the plasmid.

TABLE 62
Fold expression as compared to beta casein for BC-BC-BC-BC constructs

| | | | | Percent of events that had fold | |
|---|---|---|---|---|---|
| Plasmid | | Protein(s) | | increase of (compared to BC): | |
| ID | Cassette detail | abbreviation | Design | 0-17 fold | >17 fold |
| AR15-231 | GmSeed2:sig2:OBC-T5:OBC-T4:OBC- | BC-BC-BC- | — | 96.2% | 3.8% |
| | T3:OBC-T2:AtHSP T:AtUbi10T | BC | | | |
| AR14-14 | Cas9(KO Protease1)(GmSeed2:sig2:OBC- | BC-BC-BC- | KO Protease1- | 57.1% | 42.9% |
| | T5:OBC-T4:OBC-T3:OBC-T2:AtHSP | BC | Peptidase A1 | | |
| | T:AtUbi10T) | | domain-containing | | |
| AR14-18 | Cas9(KO Protease9)(GmSeed2:sig2:OBC- | BC-BC-BC- | KO Protease9- | 20.0% | 80.0% |
| | T5:OBC-T4:OBC-T3:OBC-T2:AtHSP | BC | serine-type protease | | |
| | T:AtUbi10T) | | | | |
| AR14-19 | Cas9(KO Protease3&4) - | BC-BC-BC- | KO Protease3&4 | 25.0% | 75.0% |
| | (GmSeed2:sig2:OBC-T5:OBC-T4:OBC- | BC | | | |
| | T3:OBC-T2:AtHSP T:AtUbi10T) | | | | |
| AR15-341 | (GmSeed2:sig2:OBC-T5:OBC-T4:OBC- | BC-BC-BC- | Coexpression of the | 0.0% | 100.0% |
| | T3:OBC-T2:AtHSP T:AtUbi10T) | BC/CYS | protease inhibitor | | |
| | (GmCEP1:sig12:SICYS8:arcT) | | CYS (GmCEP) | | |
| AR15-342 | (GmSeed2:sig2:OBC-T5:OBC-T4:OBC- | BC-BC-BC- | Coexpression of the | 20.0% | 80.0% |
| | T3:OBC-T2:AtHSP T:AtUbi10T) | BC/CYS | protease inhibitor | | |
| | (GmGRD:sig12:SICYS8:arcT) | | CYS (GmGRD) | | |

TABLE 63
Fold expression as compared to beta casein for BC-BC-KCN-LG constructs

| | | | | Percent of events that had fold | |
|---|---|---|---|---|---|
| Plasmid | | Protein(s) | | increase of (compared to BC): | |
| ID | Cassette detail | abbreviation | Design | 0-60 fold | >60 fold |
| AR15-233 | GmSeed2:sig2:OBC-T3:OBC-T2:OKC1- | BC-BC-KCN- | — | 68.4% | 31.6% |
| | T:OLG1:AtHSP T:AtUbi10T | LG | | | |
| AR14-07 | Cas9(KO Protease1)(GmSeed2:sig2:OBC- | BC-BC-KCN- | KO Protease1- | 20.0% | 80.0% |
| | T3:OBC-T2:OKC1-T:OLG1:AtHSP | LG | Peptidase A1 | | |
| | T:AtUbi10T) | | domain-containing | | |
| | | | protein | | |
| AR14-08 | Cas9(KO Protease3)(GmSeed2:sig2:OBC- | BC-BC-KCN- | KO Protease3- | 0.0% | 100.0% |
| | T3:OBC-T2:OKC1-T:OLG1:AtHSP | LG | Cysteine proteinase | | |
| | T:AtUbi10T) | | | | |
| AR14-09 | Cas9(KO Protease4)(GmSeed2:sig2:OBC- | BC-BC-KCN- | KO Protease 4- 34 | 25.0% | 75.0% |
| | T3:OBC-T2:OKC1-T:OLG1:AtHSP | LG | kDa maturing seed | | |
| | T:AtUbi10T) | | protein | | |
| AR14-12 | Cas9(KO Protease3&4)(GmSeed2:sig2:OBC- | BC-BC-KCN- | KO Protease3&4 | 33.3% | 66.7% |
| | T3:OBC-T2:OKC1-T:OLG1:AtHSP | LG | | | |
| | T:AtUbi10T) | | | | |
| AR15-343 | (GmSeed2:sig2:OBC-T3:OBC-T2:OKC1- | BC-BC-KCN- | Coexpression of | 37.5% | 62.5% |
| | T:OLG1:AtHSP T:AtUbi10T) | LG/CYS | protease inhibitor | | |
| | (PvPhas:arcUTR:sig10:SICYS8:arcT) | | CYS (PvPhas) | | |
| AR15-344 | (GmSeed2:sig2:OBC-T3:OBC-T2:OKC1- | BC-BC-KCN- | Coexpression of the | 0.0% | 100.0% |
| | T:OLG1:AtHSP T:AtUbi10T) | LG/CYS | protease inhibitor | | |
| | (GmCEP1:sig12:SICYS8:arcT) | | CYS (GmCEP) | | |
| AR15-345 | (GmSeed2:sig2:OBC-T3:OBC-T2:OKC1- | BC-BC-KCN- | Coexpression of the | 0.0% | 100.0% |
| | T:OLG1:AtHSP T:AtUbi10T) | LG/CYS | protease inhibitor | | |
| | (GmGRD:sig12:SICYS8:arcT) | | CYS (GmGRD) | | |

### Example 22: Higher Yield of Milk Protein Produced Per Acre of Engineered Plants

The genetically engineered plants of the disclosure will enable the production of recombinant milk proteins at significant quantities per acre.

For example, the concentration of total protein per soybean seed is generally in the range of 33% to 39%, with an approximate mean of 35% (but studies indicate that the range can be from around 27% to around 45%).

The genetic modifications taught in the present disclosure are able to achieve a range of expression levels of recombinant milk protein in a genetically modified seed, as a percentage of total protein per soybean seed.

Assuming a mean of 35% total protein per soybean seed, then the below Table 64 illustrates pounds of recombinant milk protein per acre, at different expression levels and soybean yield rates, which are achievable utilizing the genomic engineering techniques taught herein.

To construct Table 64, the following formula was utilized: (Y Bushels/acre)×(60 pounds soybeans/bushel)×(35 pounds total protein/100 pounds soybeans)×(E pounds recombinant protein/100 pounds total protein)=pounds of recombinant protein per acre, where E is the expression level of the recombinant protein and Y is the yield of soybeans per acre.

Further, based on the above formula, one can derive the following formula that gives the amount of recombinant protein produced per seed: (1 pound soybeans/2800 soybeans)×(35 pounds total protein/100 pounds soybeans)×(E pounds recombinant protein/100 pounds total protein)×(453600 mg/lb)=mg recombinant protein per seed. For example, if using a 1% expression level, then the equation would be: (1 pound soybeans/2800 soybeans)×(35 pounds total protein/100 pounds soybeans)×(1 pounds recombinant protein/100 pounds total protein)×(453600 mg/lb)=0.56 mg recombinant protein per seed. This is assuming 162 mg average for a soybean seed. If, however, one had a lower average weight of soybean seed, then the recombinant protein produced per seed would be different. For instance, consider an average seed weight of 100 mg, with 1% expression level, with 35% protein content, would give 0.35 mg recombinant protein per seed. One of skill in the art would be able to utilize the formula taught herein to determine the recombinant protein produced per seed.

The USDA's National Agricultural Statistics Service (USDA NASS) estimates that the average soybean yield per acre across the United States was 50.2 bushels per acre in 2020. Specifically, soybean production for 2020 totaled 4.14 billion bushels, up 16% from 2019. With record high yields in Indiana, Kentucky, Mississippi, Missouri, New Jersey, and Tennessee, and the average soybean yield is estimated at 50.2 bushels per acre, 2.8 bushels above 2019.

Thus, as an example, assuming an average 50 bushel per acre crop, and an engineered soybean plant that expresses 5.0% recombinant milk protein (assuming a mean total protein content of 35% per soybean seed), then one would achieve 52.5 pounds of recombinant milk protein per acre, utilizing the genetic modifications taught herein (relevant row shaded grey and relevant cell bolded below in Table 64).

Determination of recombinant protein weight in transgenic seeds

Transgenic seed crops are collected for determination of recombinant protein weight. Seeds are processed as previously described and total protein weight of seed mass is determined. For example seeds will be macerated, and soluble protein will be extracted via alcohol or acid precipitation. Total protein weight will be measured, and relative content of milk proteins and second proteins will be measured to calculate weight yield, and relative content of proteins of interest.

### Example 23: Coloring of the Testa with Anthocyanin

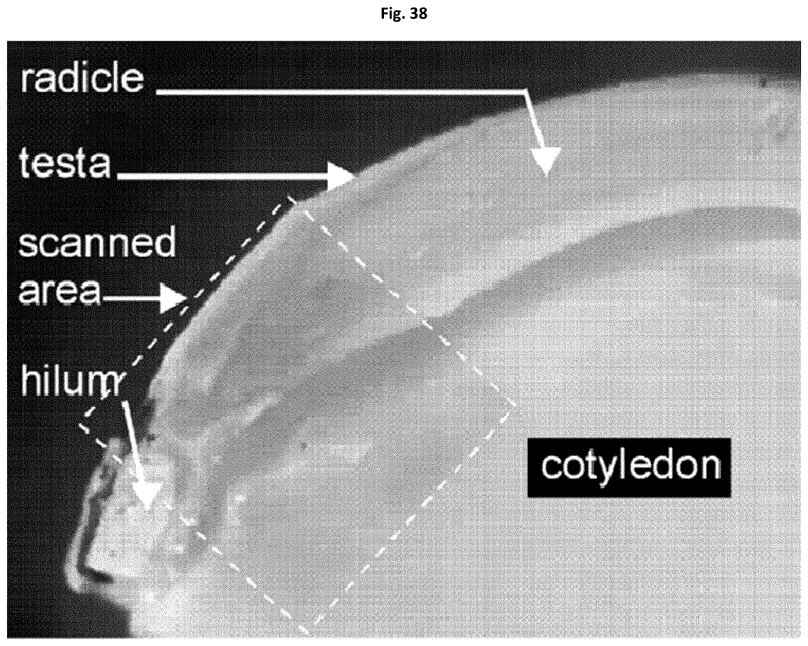

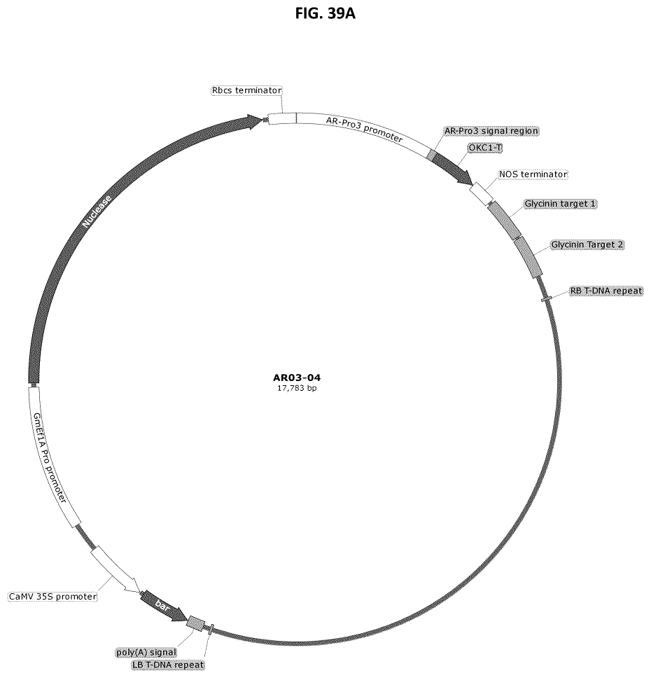

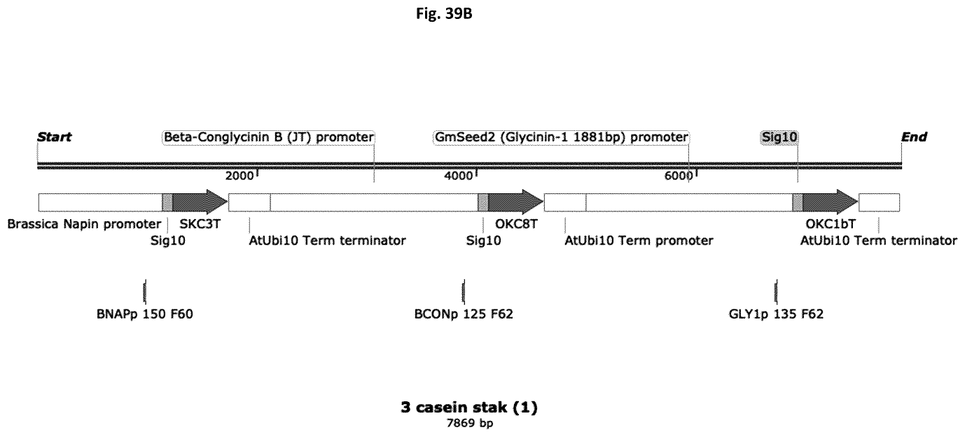

The testa of soybean, FIG. 38, is colored with anthocyanin to allow tracing and/or identification of transgenic plants (e.g., soybean). Similar methods can be applied to additional pigments or metabolites including but not limited to flavonoids, carotenoids, alkaloids.

An exemplary pigment accumulation site is the palisade layer of the testa. Accumulation in the parenchyma layer might also be done. Based on the public transcriptome analysis of seventeen compartments of the early maturation stage of the soybean seeds (GE46096), 12 seed coat specific genes were selected with specific expression in the palisade layer, parenchyma layer or full seed coat layers. qPCR was carried out to validate the expressions of the selected genes and 9 out of the 12 promoters were chosen to drive the expression of components of the pigmentation cassette, Table 65.

TABLE 65
Exemplary seed coat specific promoters

| | | | | | Chosen |
|---|---|---|---|---|---|
| SEQ | | Gene | | Seed Coat | for |
| ID | Gene ID | Name | Description | Layer | soybean |
| 861 | Glyma11g03010 | GmANS3 | Anthocyanin | Palisade | Yes |
| | | | Synthase 3 | Layer | |
| 862 | Glyma01g42350 | GmANS2 | Anthocyanin | Palisade | Yes |
| | | | Synthase 2 | Layer | |
| 863 | Glyma03g40310 | GmPAL1 | Function unknown | Palisade | No |
| | | | protein | Layer | |
| 864 | Glyma01g26230 | GmTAU8 | Glutathione S- | Palisade | Yes |
| | | | transferase 8 | Layer | |
| 865 | Glyma14g07940 | GmDFR1 | Dihydroflavonol-4- | Palisade | Yes |
| | | | reductase 1 | Layer | |
| 866 | Glyma06g00680 | GmPAL2 | Function unknown | Palisade | Yes |
| | | | protein | Layer | |
| 867 | Glyma07g28940 | GmSCB1 | Seed Coat BURP- | Parenchyma | Yes |
| | | | domain protein 1 | Layer | |
| 868 | Glyma08g19580 | GmSAG29 | Senescence | Parenchyma | Yes |
| | | | associated gene 29- | Layer | |
| | | | like | | |
| 869 | Glyma06g02500 | GmSCS1 | Seed Coat | Parenchyma | Yes |
| | | | Subtilisin 1 | Layer | |
| 870 | Glyma14g34480 | GmBX | Beta-glucosidase | Parenchyma | No |
| | | | | Layer | |
| 871 | Glyma09g30910 | GmBG | Xylan 1 4-beta- | Parenchyma | No |
| | | | xylosidase | Layer | |
| 872 | Glyma06g42780 | GmCEP1S | KDEL-tailed | Parenchyma | Yes |
| | | | cysteine | Layer | |
| | | | endopeptidase | | |
| | | | CEP1-like | | |

### Transformation Strategies

To modify a testa to express anthocyanin three or four transformation strategies can be employed: design of an anthocyanin-expressing cassette and dairy-expressing cassette in the same T-DNA so the two traits will co-segregate, as shown in FIG. 36A; generation of a stable pigmented line that will be crossed with a stable high dairy-expressing line, as depicted in FIG. 361B; generation of a stable pigmented line that will be re-transformed with the construct containing the dairy-expressing cassette; or generation of a stable dairy-expressing line that will be re-transformed with the construct containing the pigmentation cassette.

### Molecular Strategies to Induce Pigmentation

Strategy 1: Overexpression of AHB transcription factors (TTs) in the seed coat

Seven MYB transcription factors that induce anthocyanin accumulation in plants were selected to be expressed behind the testa specific promoters of Table 65.

TABLE 66
Selected MYB and bHLH transcription factor genes

| Nucleic Acid | | | | | |
|---|---|---|---|---|---|
| SEQ ID; and | | | | | |
| Amino Acid | | | | | Chosen |
| SEQ ID, | | | | | for |
| respectively | Gene ID | Gene Name | Description | Species | soybean |
| 873; 880 | AT1G56650 | AtMybA | R2R3-MYB TF | Arabidopsis | Yes |
| | | | Arabidopsis | thaliana | |
| 874; 881 | KT992776 | MoroMybA | R2R3-MYB TF | Blood orange | Yes |
| | | | Blood Orange | (Citrus sinensis) | |
| 875; 882 | JX470201 | VvMybA | R2R3-MYB TF | Grape | Yes |
| | | | Grape | (Vitis vinifera) | |
| 876; 883 | KT992773 | PamMybA.1 | R2R3-MYB TF | Plum | Yes |
| | | | Plum | (Prunus americana) | |
| 877; 884 | KT992775 | PamMybA.5 | R2R3-MYB TF | Plum | Yes |
| | | | Plum | (Prunus americana) | |
| 878; 885 | Glyma02g16670 | GmMYBA2 | R2R3-MYB TF | Soybean | Yes |
| | | | Soybean | (Glycine max) | |
| 879; 886 | Glyma09g36983 | GmTT8a | bHLH TF | Soybean | Yes |
| | | | Soybean | (Glycine max) | |

The R2R3 MYB transcription factor can function alone or together with basic helix-loop-helix (bTILH) transcription factor to control anthocyanin accumulation by turning on the transcription of the majority of the genes involved in anthocyanin biosynthesis. Overexpression of MYB TFs can overwrite the pigmentation pathway in yellow-coated soybean cultivars and lead to anthocyanin accumulation.

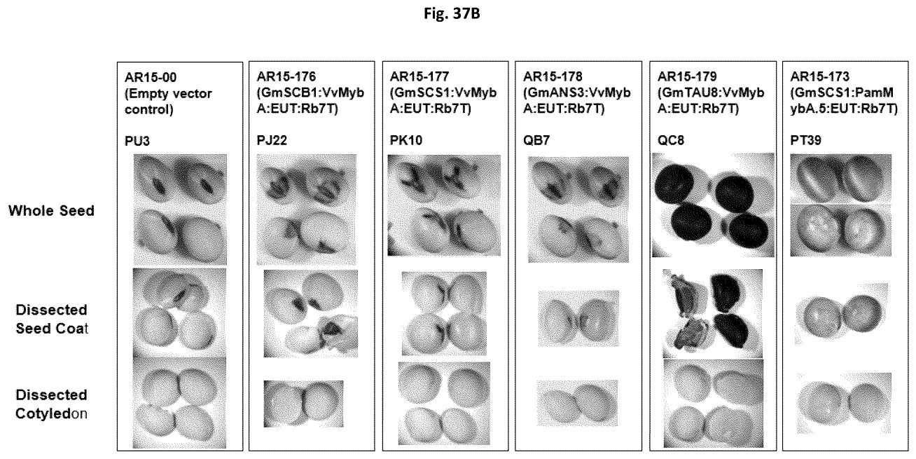

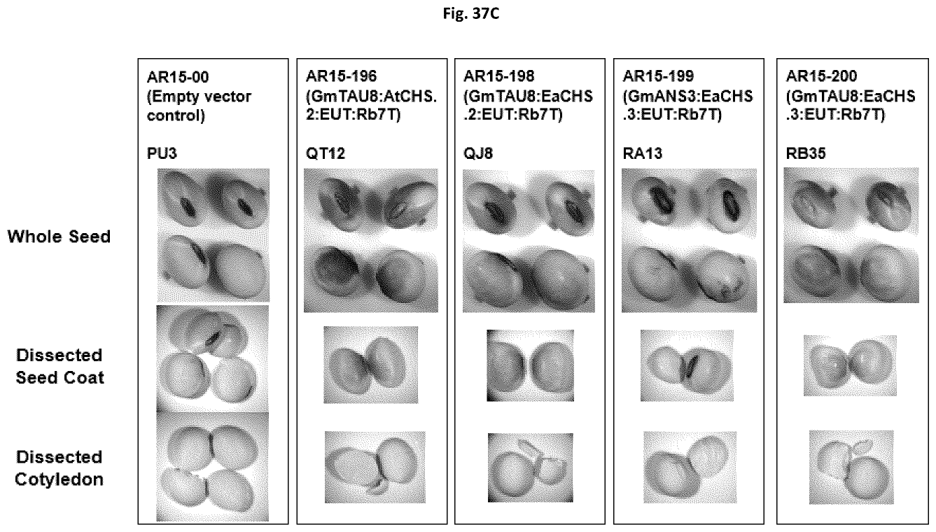

Anthocyanins were successfully accumulated in the seed coat of soybeans, FIG. 37A. Once genetically stabilized, these lines were crossed with dairy expression lines (or re-transformed with dairy-expressing lines), with any of the dairy lines described herein. Results of stable pigmented seeds, are shown in FIG. 37B for the Myb factors.

Strategy 2: Chalcone synthase expression for soy lines that have dominant WI or T allele

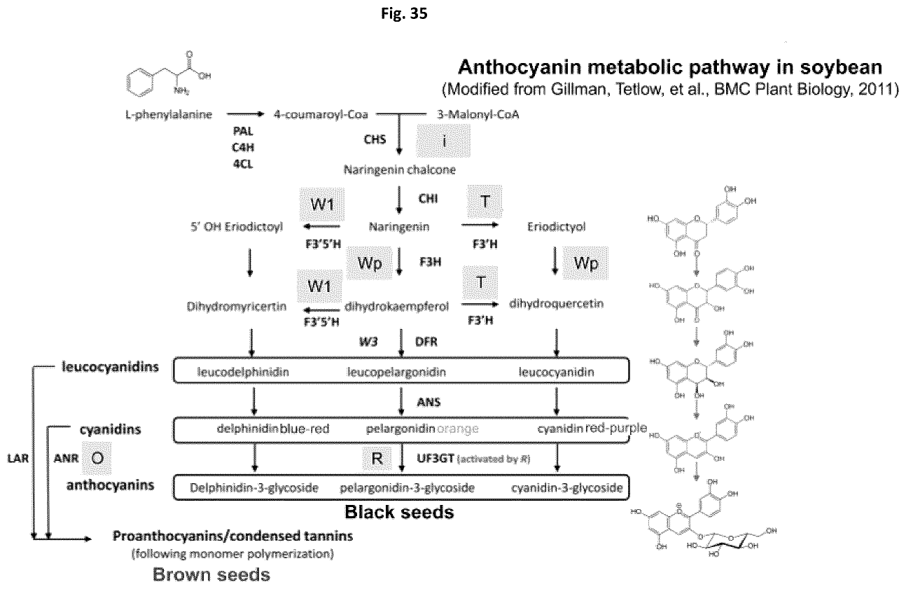

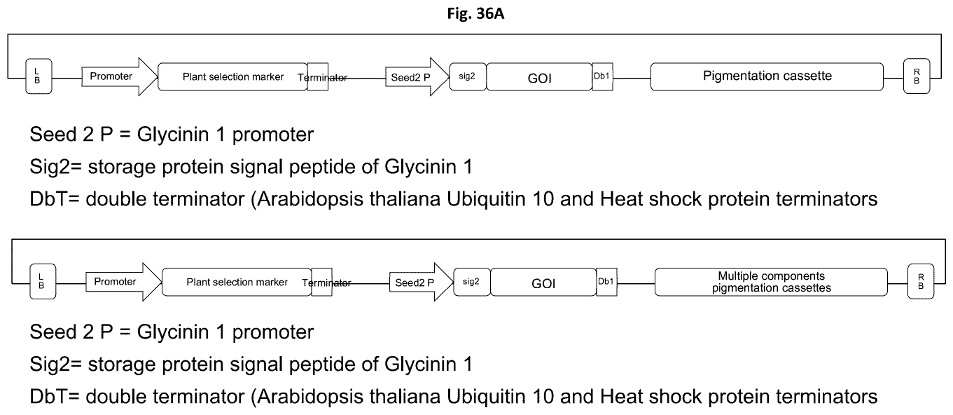

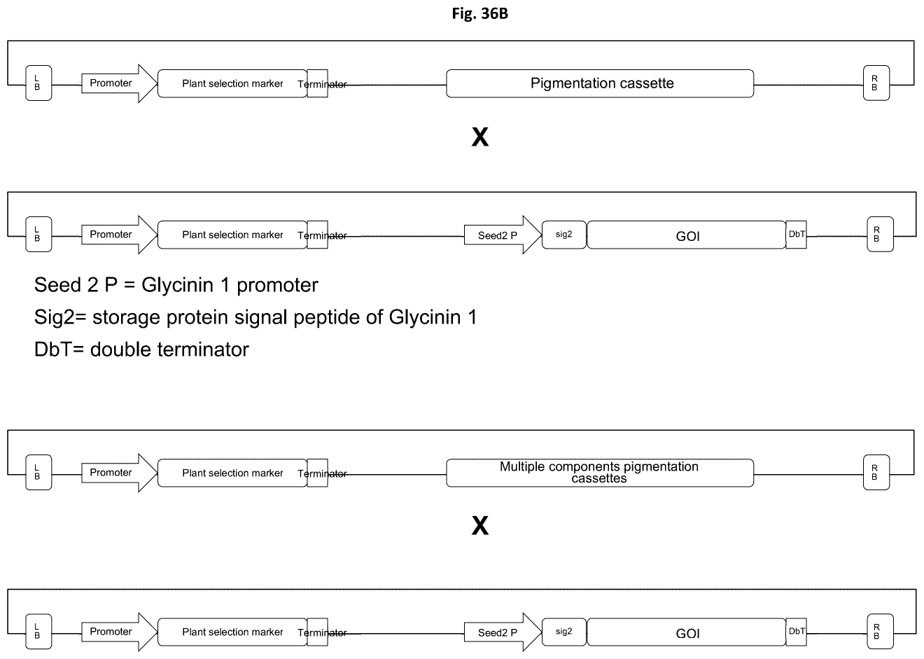

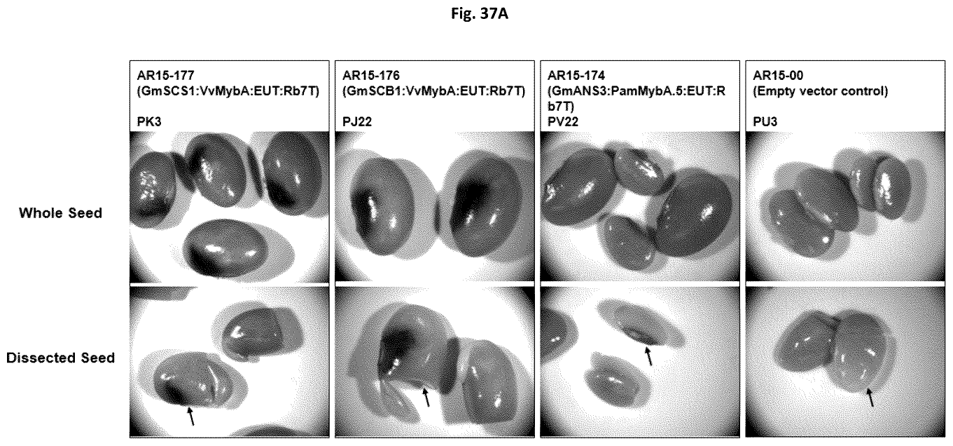

Yellow-coated soybean cultivars contain dominant I or II alleles, which inhibit the production and accumulation of pigments via post transcriptional gene silencing (PTGS) of chalcone synthase (CHS) genes (Senda, M., et al., Suppressive mechanism of seed coat pigmentation in yellow soybean. Breed. Sci. 61, 523-530 (2011)). This silencing effect blocks an essential upstream step in the anthocyanin biosynthesis (FIG. 35). For soybean cultivars containing intact downstream biosynthesis genes (dominant W1 or T allele), the silencing effect by the I allele that prevents pigmentation, can be overcome by expressing a codon-optimized version of the CHS gene. Functionally conserved CHS from other plant species might as well be more efficient to overcome the PTGS.

Three codon optimization versions were created for the most abundantly expressed chalcone synthase gene in soybean, CHS7, see Table 67. In addition, a chalcone synthase from Arabidopsis and Horsetail were also selected to create codon optimized versions and to be expressed behind the testa specific promoters of Table 65.

TABLE 67
Codon optimized chalcone synthase genes

| Nucleic Acid | | | | | |
|---|---|---|---|---|---|
| SEQ ID; and | | | | | |
| Amino Acid | | | | | Chosen |
| SEQ ID, | | | | | for |
| respectively | Gene ID | Gene Name | Description | Species | soybean |
| 887; 896 | Glyma01g43880 | GmCHS7.1 | Codon optimized | Soybean | Yes |
| | | | soybean chalcone | (Glycine max) | |
| | | | synthase 7, version 1 | | |
| 888; 897 | Glyma01g43880 | GmCHS7.2 | Codon optimized | Soybean | No |
| | | | soybean chalcone | (Glycine max) | |
| | | | synthase 7, version 2 | | |
| 889; 898 | Glyma01g43880 | GmCHS7.3 | Codon optimized | Soybean | No |
| | | | soybean chalcone | (Glycine max) | |
| | | | synthase 7, version 3 | | |
| 890; 899 | AT5G13930 | AtCHS.1 | Codon optimized | Arabidopsis | No |
| | | | Arabidopsis chalcone | thaliana | |
| | | | synthase, version 1 | | |
| 891; 900 | AT5G13930 | AtCHS.2 | Codon optimized | Arabidopsis | Yes |
| | | | Arabidopsis chalcone | thaliana | |
| | | | synthase, version 2 | | |
| 892; 901 | AT5G13930 | AtCHS.3 | Codon optimized | Arabidopsis | No |
| | | | Arabidopsis chalcone | thaliana | |
| | | | synthase, version 3 | | |
| 893; 902 | AB030004 | EaCHS.1 | Codon optimized | Horsetail | No |
| | | | horsetail chalcone | (Equisetum | |
| | | | synthase, version 1 | arvense) | |
| 894; 903 | AB030004 | EaCHS.2 | Codon optimized | Horsetail | Yes |
| | | | horsetail chalcone | (Equisetum | |
| | | | synthase, version 2 | arvense) | |
| 895; 904 | AB030004 | EaCHS.3 | Codon optimized | Horsetail | Yes |
| | | | horsetail chalcone | (Equisetum | |
| | | | synthase, version 3 | arvense) | |

Images of the phenotypes of harvested seeds generated using strategy 2 for the chalcone synthase are shown in FIG. 37C. The representative images show chalcone synthase expression for soy lines that have dominant W1 or T allele.

Strategy 3: Combination of strategy 1 and 2

Also possible is a combination of strategy 1 and 2 to enhance the promotion of the whole anthocyanin biosynthesis pathway with MYB TF, cofactor bHLH, and codon optimized CHS. While MYB TF is able to function alone, co-expression with its cofactor bHLH TF can further enhance the MYB TF activity (Gao, R., et al., (2021). MYB transcription factors GmMYBA2 and GmMYBR function in a feedback loop to control pigmentation of seed coat in soybean. Journal of Experimental Botany, 72(12), 4401-4418; Lu, N., et al., (2021). Dissecting the transcriptional regulation of proanthocyanidin and anthocyanin biosynthesis in soybean (Glycine max). Plant Biotechnology Journal, 19(7), 1429-1442). The system will be more efficient if the promoters used can be activated by the MYB TF to create a feed-forward feedback loop.

### Example 24. Use of Amino Acid Rebalancing Platform for Enhanced Tissue Specific Expression of Proteins of Interest in Transgenic Plants

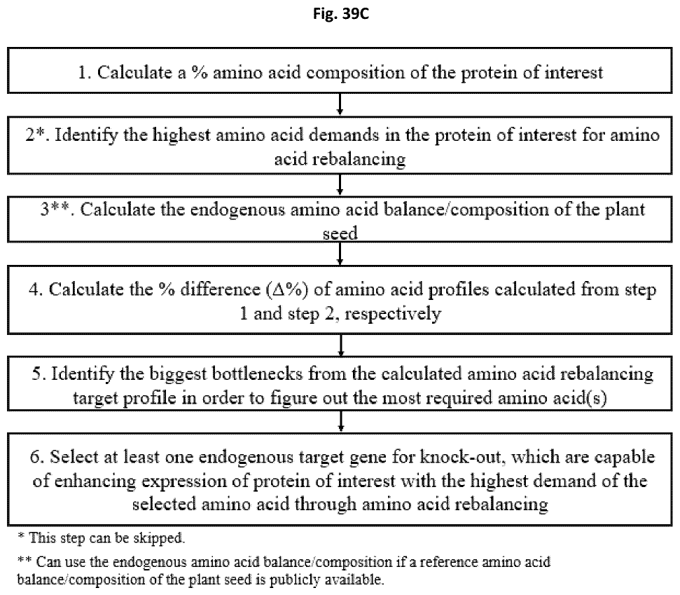

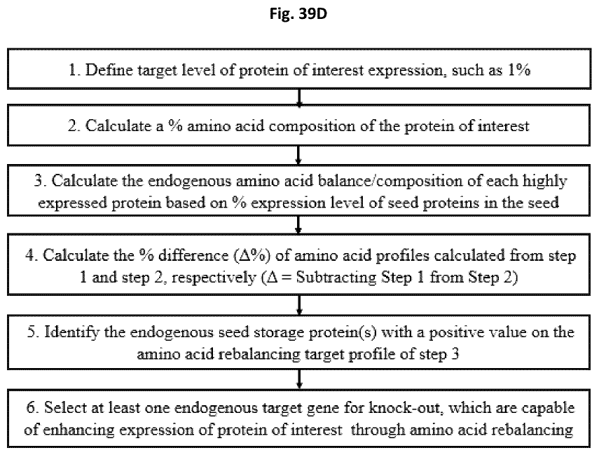

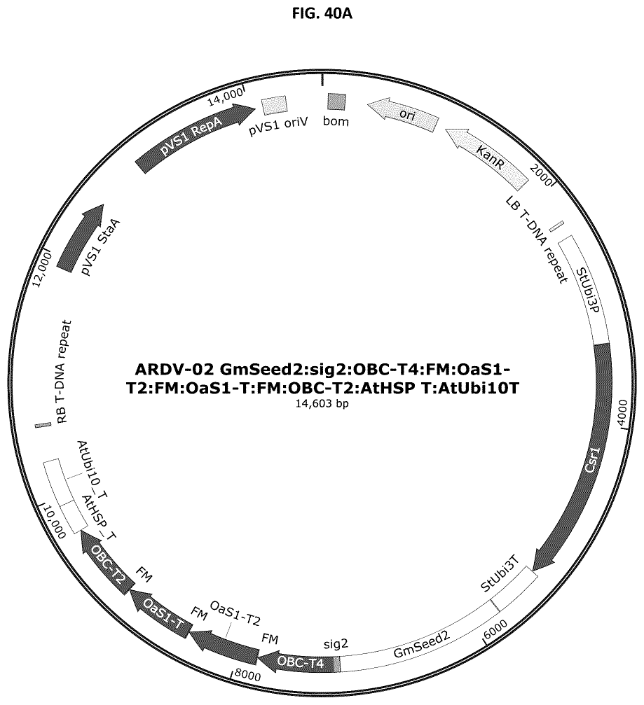

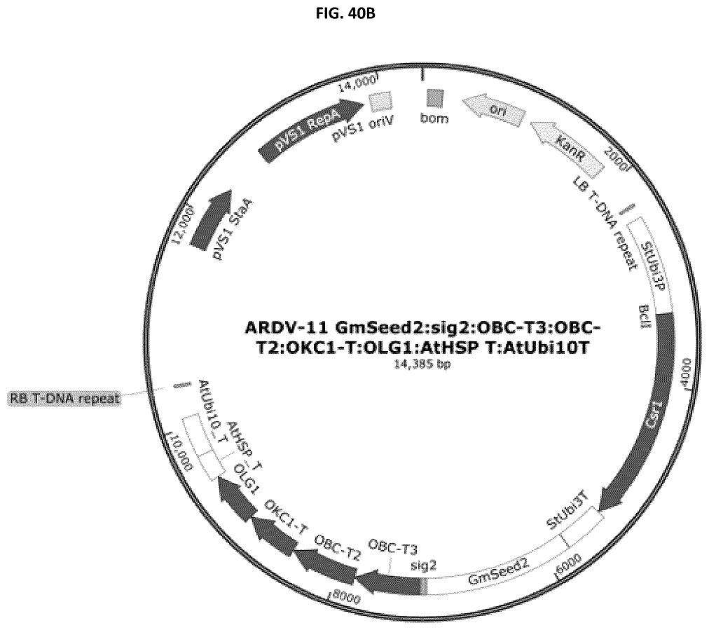

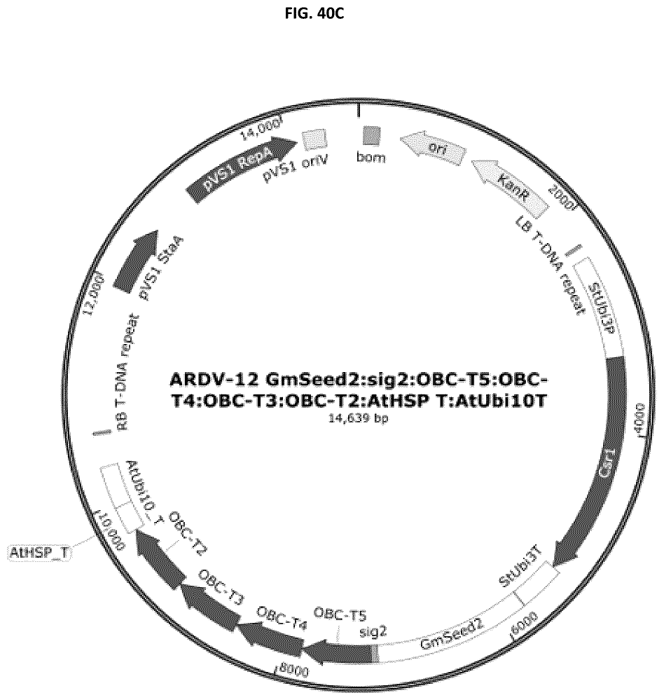

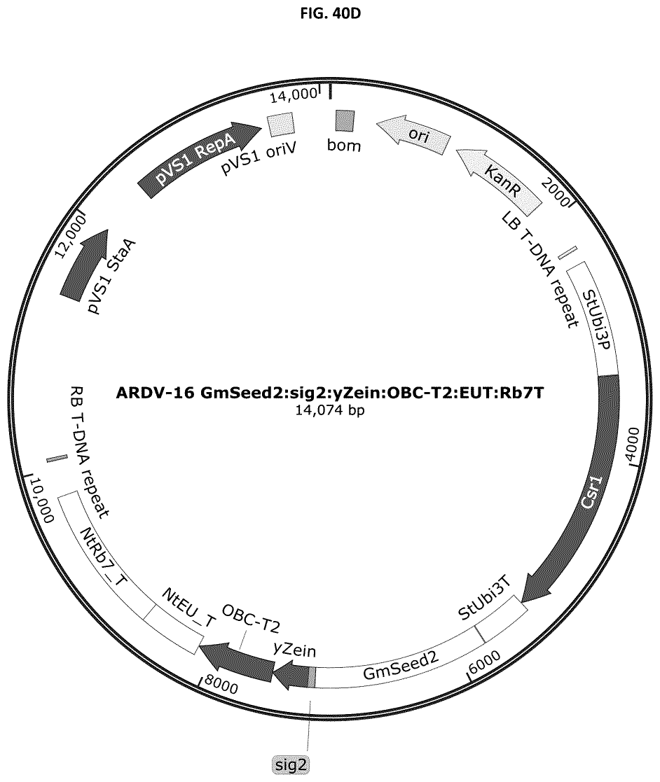

To increase the yield of recombinant protein of interest, such as milk proteins, egg proteins, meat proteins, and mammalian cell growth factors, in seeds of transgenic plants, a replacement strategy to rebalance amino acid composition in the seeds was employed where native seed-storage proteins were targeted for knock-out with gene-editing technology described herein, see FIG. 39A-FIG. 39D.

Target genes to be knocked out by a gene editing technology were selected based on a predicted demand on amino acid composition of the seed proteome. The amino acid compositions fluctuated by the missing of the endogenous target gene(s) can be compensated by expression of heterologous proteins of interest, which can rebalance amino acid compositions of the seed proteome. Using κ-casein protein as an example, the target genes for seed proteome rebalancing was determined by the following steps and also depicted in FIG. 39C-FIG. 39D:

1. Calculate a % amino acid composition of the protein of interest and identify what the highest amino acid demands in the protein of interest for amino acid rebalancing (in this example of κ-casein, Proline (11.8%) is the highest amino acid demand as shown in Table 1).

2. Calculate the endogenous amino acid balance/composition of the plant seed or using the endogenous amino acid balance/composition if a reference amino acid balance/composition of the plant seed is publicly available. For example, amino acid composition of Soybeans (Glycine max) is known in Kovalenko et al., (2006), Journal of Agricultural and Food Chemistry, 54(10), pp. 3485-3491, which is incorporated by reference in its entirety.

3. Check where the biggest bottlenecks are and identify an amino acid rebalancing target profile by calculating the % difference (Δ %) of amino acids between the protein of interest and the endogenous amino acid balance provided by the soybean seed in order to figure out the most required amino acid(s).

4. Select an endogenous target gene for knock-out, which are capable of enhancing expression of protein of interest with the highest demand of the selected amino acid for amino acid rebalancing. Exemplary cassettes targeting various genes are provided in Table 76 below. Proline is the selected amino acid for rebalancing amino acid in this example. Tables 68 and 69 are provided to present percentage of amino acid compositions of gene models encoding seed storage proteins.

Using κ-casein protein for example, it was determined that the amino acid, Proline, showed the highest difference in the amino acid compositions between κ-casein and soybean seeds. The most prominent seed storage proteins based on RNAseq (Gunadi et al. 2016 Plant Cell, Tissue and Organ Culture (PCTOC), 127(1), pp. 145-160) are listed in Tables 68 and 69.

The predicted bottlenecking amino acid is proline as described above. In one example, Glycinin 4 and Glycinin 5 are the two members of the group-2 subunit of Glycinin which is hexamers (11S). As Glycinin 4 and Glycinin 5 are paralogs with 79.65% identity in CDS. These genes encoding both proteins can be chosen for knock-out because (1) their protein sequences are the two most proline rich and (2) their sequence similarity facilitates multiplex gene-editing where individual editing targets could impact both gene models.

Out of the most prominent seed storage proteins, the proteins coded by Glyma.13G123500 (Glycinin 5), and Glyma.10G037100 (Glycinin 4), have the highest percentage in amino acid composition, at 7.16% for Proline and 6.75% for Proline, respectively, as presented in Table 68. Once genetic deficiencies of these two genes are imposed by gene-editing techniques (e.g. CRISPR/Cas systems), the transgenic seeds have more free amino acid pool for Proline, which allows for enhanced expression of foreign protein of interest possessing high Proline composition, and therefore accumulation of the foreign protein increases for amino acid rebalancing.

TABLE 68
Reference Amino Acid (AA) Percentage in
Soybean Seed and Protein of Interest

| | Total Soybean Seed* | | κ-Casein | | |
|---|---|---|---|---|---|
| AA | Mean % | Relative | No. | Relative | |
| Composition | Dry Basis | 20 AA % | of AA | 20 AA % | Δ %** |
| Ala | 1.79% | 3.61% | 14 | 8.30% | −4.69% |
| Arg | 3.17% | 6.40% | 5 | 3.00% | 3.40% |
| Asn | — | 7.20†% | 8 | 4.70% | 2.50% |
| Asp | 4.79% | 9.66% | 4 | 2.40% | 7.26% |
| Cys | 0.7% | 1.41% | 2 | 1.20% | 0.21% |
| Gln | — | 9.35†% | 15 | 8.90% | 0.45% |
| Glu | 7.66% | 15.45% | 12 | 7.10% | 8.35% |
| Gly | 1.77% | 3.57% | 2 | 1.20% | 2.37% |
| His | 1.15% | 2.32% | 3 | 1.80% | 0.52% |
| Ile | 1.94% | 3.91% | 12 | 7.10% | −3.19% |
| Leu | 3.26% | 6.58% | 8 | 4.70% | 1.88% |
| Lys | 2.69% | 5.43% | 9 | 5.30% | 0.13% |
| Met | 0.61% | 1.23% | 2 | 1.20% | 0.03% |
| Phe | 2.16% | 4.36% | 4 | 2.40% | 1.96% |
| Pro | 2.04% | 4.12% | 20 | 11.80% | −7.68% |
| Ser | 1.92% | 3.87% | 13 | 7.70% | −3.83% |
| Thr | 1.62% | 3.27% | 15 | 8.90% | −5.63% |
| Trp | 0.5% | 1.01% | 1 | 0.60% | 0.41% |
| Tyr | 1.53% | 3.09% | 9 | 5.30% | −2.21% |
| Val | 2.06% | 4.16% | 11 | 6.50% | −2.34% |
| Total | 43.16% | 100.00% | 169 | 100.00% | |

*Kovalenko, I. V., Rippke, G. R. and Hurburgh, C. R., 2006. Journal of Agricultural and Food Chemistry, 54(10), pp. 3485-3491.
†Utsumi, S., Matsumura, Y. & Mori, T. (1997) in Food Proteins and Their Applications, eds. Damodaran, S. & Paraf, A. (Dekker, New York), pp. 257-291.
**Percent (%) Difference = Relative Amino Acid (%) of total Soybean Seed − Relative Amino Acid (%) of κ-casein of interest.

TABLE 69
Percent of amino acid compositions of gene models encoding seed storage proteins.

| | Target Protein | | | | | |
|---|---|---|---|---|---|---|
| | β- | β- | β- | | | |
| | conglycinin | conglycinin | conglycinin | Cysteine | | |
| | α | α′ | β | Protease | Glycinin 1 | Glycinin 2 |
| | Gene ID (Glyma2.0) | | | | | |
| | Glyma.20G148400, | | Glyma.20g146200, | | | |
| | Glyma.20g148300 | Glyma.10G246300 | Glyma.20g148200 | Glyma.08G116300 | Glyma.03G163500 | LOC547900 |
| | AA Composition | | | | | |
| | Relative | Relative | Relative | Relative | Relative | Relative |
| | 20 AA % | 20 AA % | 20 AA % | 20 AA % | 20 AA % | 20 AA % |
| Ala | 4.63 | 4.35 | 5.24 | 6.07 | 5.86 | 6.80 |
| Arg | 7.93 | 6.60 | 7.06 | 3.43 | 5.45 | 5.98 |
| Asn | 6.78 | 6.28 | 7.52 | 4.22 | 7.47 | 8.25 |
| Asp | 4.63 | 4.35 | 4.78 | 6.07 | 3.43 | 3.71 |
| Cys | 0.83 | 0.81 | 0.23 | 1.85 | 2.22 | 2.06 |
| Gln | 7.93 | 8.53 | 7.52 | 4.22 | 9.70 | 10.52 |
| Glu | 13.06 | 13.04 | 8.43 | 7.39 | 8.28 | 7.63 |
| Gly | 4.30 | 4.83 | 4.33 | 8.18 | 7.27 | 7.22 |
| His | 1.32 | 3.54 | 1.82 | 3.69 | 1.62 | 0.82 |
| Ile | 5.12 | 4.51 | 5.92 | 5.54 | 5.25 | 4.74 |
| Leu | 8.93 | 7.73 | 10.48 | 8.18 | 7.47 | 7.84 |
| Lys | 5.95 | 6.92 | 4.78 | 6.86 | 5.05 | 3.92 |
| Met | 0.50 | 0.64 | 0.46 | 1.58 | 1.41 | 1.65 |
| Phe | 4.96 | 4.83 | 6.83 | 3.43 | 4.85 | 4.54 |
| Pro | 6.61 | 5.64 | 5.24 | 2.64 | 5.86 | 5.36 |
| Ser | 7.44 | 7.57 | 7.74 | 9.50 | 6.87 | 6.60 |
| Thr | 1.82 | 1.93 | 2.51 | 4.75 | 4.04 | 3.71 |
| Trp | 0.33 | 0.48 | 0.00 | 2.64 | 0.81 | 0.82 |
| Tyr | 2.48 | 2.42 | 2.73 | 4.75 | 2.22 | 2.27 |
| Val | 4.46 | 4.99 | 6.38 | 5.01 | 4.85 | 5.57 |
| Total | 100.00% | 100.00% | 100.00% | 100.00% | 100.00% | 100.00% |

TABLE 70
Percent of amino acid compositions of gene models encoding seed storage proteins.

| | Target Protein | | | |
|---|---|---|---|---|
| | Glycinin | Glycinin | Glycinin | Glycinin |
| | 3 | 4 | 5 | 7 |
| | Gene ID | | | |
| | Glyma.19G164900 | Glyma.10G037100 | Glyma.13G123500 | Glyma.19G164800 |
| | AA Composition | | | |
| | Relative | Relative | Relative | Relative |
| | 20 AA % | 20 AA % | 20 AA % | 20 AA % |
| Ala | 6.24 | 4.26 | 4.09 | 5.60 |
| Arg | 6.03 | 6.39 | 6.13 | 6.34 |
| Asn | 7.48 | 5.68 | 5.93 | 5.22 |
| Asp | 3.33 | 5.68 | 4.70 | 2.61 |
| Cys | 2.29 | 1.42 | 1.64 | 2.43 |
| Gln | 10.19 | 9.06 | 8.79 | 4.48 |
| Glu | 7.90 | 9.77 | 8.79 | 10.82 |
| Gly | 6.65 | 6.75 | 7.57 | 8.40 |
| His | 1.25 | 2.66 | 3.27 | 4.48 |
| Ile | 4.99 | 3.73 | 3.68 | 5.22 |
| Leu | 7.48 | 7.99 | 7.57 | 7.46 |
| Lys | 3.95 | 4.80 | 3.68 | 5.97 |
| Met | 1.25 | 0.53 | 0.82 | 1.31 |
| Phe | 6.03 | 2.84 | 3.89 | 3.54 |
| Pro | 6.03 | 6.75 | 7.16 | 5.22 |
| Ser | 7.07 | 8.17 | 7.98 | 5.22 |
| Thr | 3.74 | 3.55 | 4.09 | 3.92 |
| Trp | 0.62 | 1.07 | 0.82 | 1.12 |
| Tyr | 2.08 | 2.66 | 2.86 | 2.61 |
| Val | 5.41 | 6.22 | 6.54 | 8.02 |
| Total | 100.00 | 100.00 | 100.00 | 100.00 |

| | Target Protein | | |
|---|---|---|---|
| | Kunitz- | Kunitz- | |
| | type | type | |
| | Trypsin | Trypsin | |
| | inhibitor | inhibitor | Lectin |
| | Gene ID | | |
| | Glyma.01G095000 | Glyma.08G341500 | Glyma.02G012600 |
| | AA Composition | | |
| | Relative | Relative | Relative |
| | 20 AA % | 20 AA % | 20 AA % |
| Ala | 6.90 | 5.99 | 8.07 |
| Arg | 3.94 | 4.61 | 2.11 |
| Asn | 2.96 | 4.61 | 6.32 |
| Asp | 6.90 | 7.83 | 5.96 |
| Cys | 2.46 | 2.30 | 0.00 |
| Gln | 4.43 | 2.30 | 2.46 |
| Glu | 5.42 | 6.45 | 3.16 |
| Gly | 7.88 | 7.83 | 4.56 |
| His | 0.49 | 1.38 | 2.11 |
| Ile | 6.90 | 7.37 | 5.26 |
| Leu | 9.36 | 8.76 | 11.23 |
| Lys | 5.42 | 5.99 | 5.26 |
| Met | 1.97 | 1.38 | 0.70 |
| Phe | 6.40 | 6.45 | 4.91 |
| Pro | 4.93 | 5.07 | 5.61 |
| Ser | 5.91 | 7.37 | 12.28 |
| Thr | 5.91 | 4.61 | 8.07 |
| Trp | 0.99 | 0.92 | 2.11 |
| Tyr | 2.96 | 2.30 | 1.75 |
| Val | 7.88 | 6.45 | 8.07 |
| Total | 100.00 | 100.00 | 100.00 |

In another example, this amino acid rebalancing platform strategy enables a user to fine-tune the selection of a gene to be knocked out, thereby reducing or eliminating a protein, and provides a tailored approach for the proteins of interest to be expressed. An amino acid rebalancing strategy can be applied for selection of genes to be genomically disrupted to reduce or knock out an endogenous seed protein.

Table 70 displays the percent value of each amino acid that constitutes the seed storage proteins for most expressed seed storage proteins. The percent expressed protein value was estimated by LC-MS-MS from a mature seed aqueous extract. Protein expression in seeds provided in Table 70 are taken into consideration and the six top expression seed storage proteins are presented in Table 70, which are β-conglycinin α, β-conglycinin α′, β-conglycinin β, Glycinin 1, Glycinin 4, and Kunitz-Trypsin inhibitor.

Tables 71 to Table 74 demonstrates the estimating of the percent amount of amino acid if a protein of interest is expressed at 10% in the seed. As presented in Tables 71-74, the delta difference (A %) of the estimated percentage of amino acid to each of the most abundant seed proteins from Tables 69-70 are calculated. This approach is independent of the recombinant protein that needs to be expressed, but is based on a seed amino acid rebalancing strategy. Examples are shown for the following proteins: κ-Casein (Table 72), Ovalbumin (Table 73), Vitellogenin-2 (Table 74) and a chicken fibroblast growth factor (Table 75).

A positive value (no highlight) means that if that particular seed protein is knocked out, it would release enough amino acid to buffer the production of the protein of interest expressed at 10 percent.

A negative value (highlighted in yellow) means that knocking out that specific protein will not release enough amino acids and more than one protein should be knocked out.

Two Exemplary Calculations of Amino Acid Difference are provided below;

1. Expression of κ-Casein at 10% in the seeds (Table 71).
(i) screening the protein profiles presented in Table 71 and identifying the protein the most likely to release the amino acids to enable the synthesis of κ-Casein, which would be Glycinin 1 with most of amino acids having the positive value other than proline, Threonine, and Tyrosine.
(ii) checking a negative delta amino acid presented in Table 71 for Glycinin, which is −0.16% of proline, −0.18% of Threonine and −0.14% of Tyrosine.
(iii) Other than glycinin 1 selected for the amino acid rebalancing from step (i), the protein with the closest matching profile to release enough of proline, threonine and tyrosine is either Kunitz-Trypsin (Glyma.08G341500), the β-conglycinin α, β-conglycinin α′, β-conglycinin R or Glycinin 4. Thus, knocking out one of them is sufficient along with Glycinin 1 knock-out.
(iv) Based on step (iii), the target protein for knocking out would be β-conglycinin proteins, but avoiding knocking out a homologous protein to glycinin 1.

2. Expression of Vitollegenin2 at 10% in the seeds (Table 73).
(i) screening the protein profiles presented in Table 73 and identifying the protein the most likely to release the amino acids to enable the synthesis of Vitollegenin2 is β-conglycinin α′ with most of amino acids having the positive value except for Ser.
(ii) checking a negative delta amino acid presented in Table 73 for Vitollegenin2, which is serine at 4.26%.
(iii) In this particular case, serine is in significant negative difference and a combination of protein will have to be knocked out in order to release enough serine. In Table 73, excluding β-conglycinin α′, proteins to consider for knocking out are a combination of 3-conglycinin α, β-conglycinin β, Glycinin 1 and Glycinin 4.

Table 71 to Table 74 provide information on amino acid difference between the most abundant seed storage proteins and κ-Casein (Table 67), Ovalbumin (Table 73), Vitellogenin-2 (Table 74) and a chicken fibroblast growth factor (Table 75) when each protein is expressed at 10% in soybean seeds. Using the amino acid difference information in Tables 71-74, one or more seed storage proteins-encoding genes can be selected to be knocked out for the genetic alteration, which can result in changes in the proteomes as the seed rebalances its protein content. As seeds possess intrinsic compositional plasticity resulting from the alteration of the source-sink relationship; this can be replenished by the accumulation of foreign proteins as an alternative sink protein.

The amino acid rebalancing platform can be used for the exchange of intrinsic protein loss for enhanced expression of foreign proteins that can minimize unpredictable collateral changes in the proteome but maximize predictable collateral proteome rebalancing by the method taught in the above examples.

The amino acid rebalancing platform is applicable for any of foreign proteins including, but not limited to as milk proteins, egg proteins, meat proteins, and mammalian cell growth factors as taught in the present disclosure.

TABLE 71
Percent representation of each amino acids in the most expressed storage seed protein

| | β- | β- | β- | | | | |
|---|---|---|---|---|---|---|---|
| | conglycinin | conglycinin | conglycinin | Cystein | | | |
| | α | α′ | β | Protease | Glycinin 1 | Glycinin 2 | Glycinin 3 |
| | Glyma2.0 | | | | | | |
| | Glyma.20G148400, | | Glyma.20g146200, | | | | |
| | Glyma.20g148300 | Glyma.10G246300 | Glyma.20g148200 | Glyma.08G116300 | Glyma.03G163500 | LOC547900 | Glyma.19G164900 |
| | % protein expression | | | | | | |
| | 12.3% | 18.20% | 17.61% | 0.09% | 17.33% | 1.70% | 1.10% |
| Ala | 0.57% | 0.79% | 0.92% | 0.01% | 1.02% | 0.12% | 0.07% |
| Arg | 0.98% | 1.20% | 1.24% | 0.00% | 0.95% | 0.10% | 0.07% |
| Asn | 0.84% | 1.14% | 1.32% | 0.00% | 1.30% | 0.14% | 0.08% |
| Asp | 0.57% | 0.79% | 0.84% | 0.01% | 0.60% | 0.06% | 0.04% |
| Cys | 0.10% | 0.15% | 0.04% | 0.00% | 0.39% | 0.04% | 0.03% |
| Gln | 0.98% | 1.55% | 1.32% | 0.00% | 1.68% | 0.18% | 0.11% |
| Glu | 1.61% | 2.37% | 1.48% | 0.01% | 1.44% | 0.13% | 0.09% |
| Gly | 0.53% | 0.88% | 0.76% | 0.01% | 1.26% | 0.12% | 0.07% |
| His | 0.16% | 0.64% | 0.32% | 0.00% | 0.28% | 0.01% | 0.01% |
| Ile | 0.63% | 0.82% | 1.04% | 0.00% | 0.91% | 0.08% | 0.05% |
| Leu | 1.10% | 1.41% | 1.85% | 0.01% | 1.30% | 0.13% | 0.08% |
| Lys | 0.73% | 1.26% | 0.84% | 0.01% | 0.88% | 0.07% | 0.04% |
| Met | 0.06% | 0.12% | 0.08% | 0.00% | 0.25% | 0.03% | 0.01% |
| Phe | 0.61% | 0.88% | 1.20% | 0.00% | 0.84% | 0.08% | 0.07% |
| Pro | 0.82% | 1.03% | 0.92% | 0.00% | 1.02% | 0.09% | 0.07% |
| Ser | 0.92% | 1.38% | 1.36% | 0.01% | 1.19% | 0.11% | 0.08% |
| Thr | 0.22% | 0.35% | 0.44% | 0.00% | 0.70% | 0.06% | 0.04% |
| Trp | 0.04% | 0.09% | 0.00% | 0.00% | 0.14% | 0.01% | 0.01% |
| Tyr | 0.31% | 0.44% | 0.48% | 0.00% | 0.39% | 0.04% | 0.02% |
| Val | 0.55% | 0.91% | 1.12% | 0.00% | 0.84% | 0.09% | 0.06% |

| | | | | Kunitz- | Kunitz- | |
|---|---|---|---|---|---|---|
| | Glycinin 4 | Glycinin 5 | Glycinin 7 | Trypsin | Trypsin | Lectin |
| | Glyma2.0 | | | | | |
| | Glyma.10G037100 | Glyma.13G123500 | Glyma.19G164800 | Glyma.01G095000 | Glyma.08G341500 | Glyma.02G012600 |
| | % protein expression | | | | | |
| | 12.66% | 2.35% | 0.00% | 1.73% | 14.39% | 0.82% |
| Ala | 0.54% | 0.10% | 0.00% | 0.12% | 0.86% | 0.07% |
| Arg | 0.81% | 0.14% | 0.00% | 0.07% | 0.66% | 0.02% |
| Asn | 0.72% | 0.14% | 0.00% | 0.05% | 0.66% | 0.05% |
| Asp | 0.72% | 0.11% | 0.00% | 0.12% | 1.13% | 0.05% |
| Cys | 0.18% | 0.04% | 0.00% | 0.04% | 0.33% | 0.00% |
| Gln | 1.15% | 0.21% | 0.00% | 0.08% | 0.33% | 0.02% |
| Glu | 1.24% | 0.21% | 0.00% | 0.09% | 0.93% | 0.03% |
| Gly | 0.85% | 0.18% | 0.00% | 0.14% | 1.13% | 0.04% |
| His | 0.34% | 0.08% | 0.00% | 0.01% | 0.20% | 0.02% |
| Ile | 0.47% | 0.09% | 0.00% | 0.12% | 1.06% | 0.04% |
| Leu | 1.01% | 0.18% | 0.00% | 0.16% | 1.26% | 0.09% |
| Lys | 0.61% | 0.09% | 0.00% | 0.09% | 0.86% | 0.04% |
| Met | 0.07% | 0.02% | 0.00% | 0.03% | 0.20% | 0.01% |
| Phe | 0.36% | 0.09% | 0.00% | 0.11% | 0.93% | 0.04% |
| Pro | 0.85% | 0.17% | 0.00% | 0.09% | 0.73% | 0.05% |
| Ser | 1.03% | 0.19% | 0.00% | 0.10% | 1.06% | 0.10% |
| Thr | 0.45% | 0.10% | 0.00% | 0.10% | 0.66% | 0.07% |
| Trp | 0.13% | 0.02% | 0.00% | 0.02% | 0.13% | 0.02% |
| Tyr | 0.34% | 0.07% | 0.00% | 0.05% | 0.33% | 0.01% |
| Val | 0.79% | 0.15% | 0.00% | 0.14% | 0.93% | 0.07% |

TABLE 72
Calculation of the amino acid difference between κ-Casein expressed at
10% and the most abundant seed storage proteins in Soybean (Δ%)

| | | | Δ%(Soy-KCN) | | | | | |
|---|---|---|---|---|---|---|---|---|
| | | | β- | β- | β- | | | |
| OKC1-T | | | conglycinin | conglycinin | conglycinin | Cystein | Glycinin | Glycinin |
| AA | % | 10.00% | α | α′ | β | Protease | 1 | 2 |
| Ala | 8.20% | 0.82% | −0.25% | −0.03% | 0.10% | −0.81% | 0.20% | −0.70% |
| Arg | 2.90% | 0.29% | 0.69% | 0.91% | 0.95% | −0.29% | 0.66% | −0.19% |
| Asn | 4.70% | 0.47% | 0.37% | 0.67% | 0.85% | −0.47% | 0.83% | −0.33% |
| Asp | 2.40% | 0.24% | 0.33% | 0.55% | 0.60% | −0.23% | 0.36% | −0.18% |
| Cys | 1.20% | 0.12% | −0.02% | 0.03% | −0.08% | −0.12% | 0.27% | −0.08% |
| Gln | 8.80% | 0.88% | 0.10% | 0.67% | 0.44% | −0.88% | 0.80% | −0.70% |
| Glu | 7.10% | 0.71% | 0.90% | 1.66% | 0.77% | −0.70% | 0.73% | −0.58% |
| Gly | 1.20% | 0.12% | 0.41% | 0.76% | 0.64% | −0.11% | 1.14% | 0.00% |
| His | 1.80% | 0.18% | −0.02% | 0.46% | 0.14% | −0.18% | 0.10% | −0.17% |
| Ile | 7.10% | 0.71% | −0.08% | 0.11% | 0.33% | −0.71% | 0.20% | −0.63% |
| Leu | 4.70% | 0.47% | 0.63% | 0.94% | 1.38% | −0.46% | 0.83% | −0.34% |
| Lys | 5.30% | 0.53% | 0.20% | 0.73% | 0.31% | −0.52% | 0.35% | −0.46% |
| Met | 1.80% | 0.18% | −0.12% | −0.06% | −0.10% | −0.18% | 0.07% | −0.15% |
| Phe | 2.40% | 0.24% | 0.37% | 0.64% | 0.96% | −0.24% | 0.60% | −0.16% |
| Pro | 11.80% | 1.18% | −0.36% | −0.15% | −0.26% | −1.18% | −0.16% | −1.09% |
| Ser | 7.60% | 0.76% | 0.16% | 0.62% | 0.60% | −0.75% | 0.43% | −0.65% |
| Thr | 8.80% | 0.88% | −0.66% | −0.53% | −0.44% | −0.88% | −0.18% | −0.82% |
| Trp | 0.60% | 0.06% | −0.02% | 0.03% | −0.06% | −0.06% | 0.08% | −0.05% |
| Tyr | 5.30% | 0.53% | −0.22% | −0.09% | −0.05% | −0.53% | −0.14% | −0.49% |
| Val | 6.50% | 0.65% | −0.10% | 0.26% | 0.47% | −0.65% | 0.19% | −0.56% |

| | Δ%(Soy-KCN) | | | | | |
|---|---|---|---|---|---|---|
| OKC1-T | Glycinin | Glycinin | Glycinin | Glycinin | Kunitz- | |
| AA | 3 | 4 | 5 | 7 | Trypsin | Lectin |
| Ala | −0.75% | −0.28% | −0.72% | −0.82% | −0.70% | 0.04% |
| Arg | −0.22% | 0.52% | −0.15% | −0.29% | −0.22% | 0.37% |
| Asn | −0.39% | 0.25% | −0.33% | −0.47% | −0.42% | 0.19% |
| Asp | −0.20% | 0.48% | −0.13% | −0.24% | −0.12% | 0.89% |
| Cys | −0.09% | 0.06% | −0.08% | −0.12% | −0.08% | 0.21% |
| Gln | −0.77% | 0.27% | −0.67% | −0.88% | −0.80% | −0.55% |
| Glu | −0.62% | 0.53% | −0.50% | −0.71% | −0.62% | 0.22% |
| Gly | −0.05% | 0.73% | 0.06% | −0.12% | 0.02% | 1.01% |
| His | −0.17% | 0.16% | −0.10% | −0.18% | −0.17% | 0.02% |
| Ile | −0.66% | −0.24% | −0.62% | −0.71% | −0.59% | 0.35% |
| Leu | −0.39% | 0.54% | −0.29% | −0.47% | −0.31% | 0.79% |
| Lys | −0.49% | 0.08% | −0.44% | −0.53% | −0.44% | 0.33% |
| Met | −0.17% | −0.11% | −0.16% | −0.18% | −0.15% | 0.02% |
| Phe | −0.17% | 0.12% | −0.15% | −0.24% | −0.13% | 0.69% |
| Pro | −1.11% | −0.33% | −1.01% | −1.18% | −1.09% | −0.45% |
| Ser | −0.68% | 0.27% | −0.57% | −0.76% | −0.66% | 0.30% |
| Thr | −0.84% | −0.43% | −0.78% | −0.88% | −0.78% | −0.22% |
| Trp | −0.05% | 0.07% | −0.04% | −0.06% | −0.04% | 0.07% |
| Tyr | −0.51% | −0.19% | −0.46% | −0.53% | −0.48% | −0.20% |
| Val | −0.59% | 0.14% | −0.50% | −0.65% | −0.51% | 0.28% |

TABLE 73
Calculation of the amino acid difference between egg Ovalbumin expressed
at 10% and the most abundant seed storage proteins in Soybean (Δ%)

| | | | Δ%(Soy-Ovalbumin) | | | | | |
|---|---|---|---|---|---|---|---|---|
| | | | β- | β- | | | | |
| Ovalbumin | | | conglycinin | conglycinin | β- | Cysteine | Glycinin | Glycinin |
| AA | % | 10.00% | α | α′ | conglycinin | Protease | 1 | 2 |
| Ala | 9.10% | 0.91% | −0.34% | −0.12% | 0.01% | −0.90% | 0.11% | −0.79% |
| Arg | 3.90% | 0.39% | 0.59% | 0.81% | 0.85% | −0.39% | 0.56% | −0.29% |
| Asn | 4.40% | 0.44% | 0.40% | 0.70% | 0.88% | −0.44% | 0.86% | −0.30% |
| Asp | 3.60% | 0.36% | 0.21% | 0.43% | 0.48% | −0.35% | 0.24% | −0.30% |
| Cys | 1.60% | 0.16% | −0.06% | −0.01% | −0.12% | −0.16% | 0.23% | −0.12% |
| Gln | 3.90% | 0.39% | 0.59% | 1.16% | 0.93% | −0.39% | 1.29% | −0.21% |
| Glu | 8.50% | 0.85% | 0.76% | 1.52% | 0.63% | −0.84% | 0.59% | −0.72% |
| Gly | 4.90% | 0.49% | 0.04% | 0.39% | 0.27% | −0.48% | 0.77% | −0.37% |
| His | 1.80% | 0.18% | −0.02% | 0.46% | 0.14% | −0.18% | 0.10% | −0.17% |
| Ile | 6.50% | 0.65% | −0.02% | 0.17% | 0.39% | −0.65% | 0.26% | −0.57% |
| Leu | 8.30% | 0.83% | 0.27% | 0.58% | 1.02% | −0.82% | 0.47% | −0.70% |
| Lys | 5.20% | 0.52% | 0.21% | 0.74% | 0.32% | −0.51% | 0.36% | −0.45% |
| Met | 4.40% | 0.44% | −0.38% | −0.32% | −0.36% | −0.44% | −0.19% | −0.41% |
| Phe | 5.20% | 0.52% | 0.09% | 0.36% | 0.68% | −0.52% | 0.32% | −0.44% |
| Pro | 3.60% | 0.36% | 0.46% | 0.67% | 0.56% | −0.36% | 0.66% | −0.27% |
| Ser | 9.80% | 0.98% | −0.06% | 0.40% | 0.38% | −0.97% | 0.21% | −0.87% |
| Thr | 3.90% | 0.39% | −0.17% | −0.04% | 0.05% | −0.39% | 0.31% | −0.33% |
| Trp | 0.80% | 0.08% | −0.04% | 0.01% | −0.08% | −0.08% | 0.06% | −0.07% |
| Tyr | 2.60% | 0.26% | 0.05% | 0.18% | 0.22% | −0.26% | 0.13% | −0.22% |
| Val | 8.00% | 0.80% | −0.25% | 0.11% | 0.32% | −0.80% | 0.04% | −0.71% |

| | Δ%(Soy-Ovalbumin) | | | | | |
|---|---|---|---|---|---|---|
| Ovalbumin | Glycinin | Glycinin | Glycinin | Glycinin | Kunitz- | |
| AA | 3 | 4 | 5 | 7 | Trypsin | Lectin |
| Ala | −0.84% | −0.37% | −0.81% | −0.91% | −0.79% | −0.05% |
| Arg | −0.32% | 0.42% | −0.25% | −0.39% | −0.32% | 0.27% |
| Asn | −0.36% | 0.28% | −0.30% | −0.44% | −0.39% | 0.22% |
| Asp | −0.32% | 0.36% | −0.25% | −0.36% | −0.24% | 0.77% |
| Cys | −0.13% | 0.02% | −0.12% | −0.16% | −0.12% | 0.17% |
| Gln | −0.28% | 0.76% | −0.18% | −0.39% | −0.31% | −0.06% |
| Glu | −0.76% | 0.39% | −0.64% | −0.85% | −0.76% | 0.08% |
| Gly | −0.42% | 0.36% | −0.31% | −0.49% | −0.35% | 0.64% |
| His | −0.17% | 0.16% | −0.10% | −0.18% | −0.17% | 0.02% |
| Ile | −0.60% | −0.18% | −0.56% | −0.65% | −0.53% | 0.41% |
| Leu | −0.75% | 0.18% | −0.65% | −0.83% | −0.67% | 0.43% |
| Lys | −0.48% | 0.09% | −0.43% | −0.52% | −0.43% | 0.34% |
| Met | −0.43% | −0.37% | −0.42% | −0.44% | −0.41% | −0.24% |
| Phe | −0.45% | −0.16% | −0.43% | −0.52% | −0.41% | 0.41% |
| Pro | −0.29% | 0.49% | −0.19% | −0.36% | −0.27% | 0.37% |
| Ser | −0.90% | 0.05% | −0.79% | −0.98% | −0.88% | 0.08% |
| Thr | −0.35% | 0.06% | −0.29% | −0.39% | −0.29% | 0.27% |
| Trp | −0.07% | 0.05% | −0.06% | −0.08% | −0.06% | 0.05% |
| Tyr | −0.24% | 0.08% | −0.19% | −0.26% | −0.21% | 0.07% |
| Val | −0.74% | −0.01% | −0.65% | −0.80% | −0.66% | 0.13% |

TABLE 74
Calculation of the amino acid difference between egg vitellogenin-2 expressed
at 10% and the most abundant seed storage proteins in Soybean (Δ%)

| | | | Δ%(Soy-vitellogenin-2) | | | | | |
|---|---|---|---|---|---|---|---|---|
| | | | β- | β- | β- | | | |
| Vitellogenin-2 | | | conglycinin | conglycinin | conglycinin | Cystein | Glycinin | Glycinin |
| AA | % | 10.00% | α | α′ | β | Protease | 1 | 2 |
| Ala | 3.20% | 0.32% | 0.25% | 0.47% | 0.60% | −0.31% | 0.70% | −0.20% |
| Arg | 5.00% | 0.50% | 0.48% | 0.70% | 0.74% | −0.50% | 0.45% | −0.40% |
| Asn | 3.20% | 0.32% | 0.52% | 0.82% | 1.00% | −0.32% | 0.98% | −0.18% |
| Asp | 2.80% | 0.28% | 0.29% | 0.51% | 0.56% | −0.27% | 0.32% | −0.22% |
| Cys | 0.00% | 0.00% | 0.10% | 0.15% | 0.04% | 0.00% | 0.39% | 0.04% |
| Gln | 1.40% | 0.14% | 0.84% | 1.41% | 1.18% | −0.14% | 1.54% | 0.04% |
| Glu | 3.70% | 0.37% | 1.24% | 2.00% | 1.11% | −0.36% | 1.07% | −0.24% |
| Gly | 2.30% | 0.23% | 0.30% | 0.65% | 0.53% | −0.22% | 1.03% | −0.11% |
| His | 6.00% | 0.60% | −0.44% | 0.04% | −0.28% | −0.60% | −0.32% | −0.59% |
| Ile | 0.90% | 0.09% | 0.54% | 0.73% | 0.95% | −0.09% | 0.82% | −0.01% |
| Leu | 1.40% | 0.14% | 0.96% | 1.27% | 1.71% | −0.13% | 1.16% | −0.01% |
| Lys | 6.90% | 0.69% | 0.04% | 0.57% | 0.15% | −0.68% | 0.19% | −0.62% |
| Met | 0.90% | 0.09% | −0.03% | 0.03% | −0.01% | −0.09% | 0.16% | −0.06% |
| Phe | 0.50% | 0.05% | 0.56% | 0.83% | 1.15% | −0.05% | 0.79% | 0.03% |
| Pro | 1.40% | 0.14% | 0.68% | 0.89% | 0.78% | −0.14% | 0.88% | −0.05% |
| Ser | 56.40% | 5.64% | −4.72% | −4.26% | −4.28% | −5.63% | −4.45% | −5.53% |
| Thr | 1.80% | 0.18% | 0.04% | 0.17% | 0.26% | −0.18% | 0.52% | −0.12% |
| Trp | 0.50% | 0.05% | −0.01% | 0.04% | 0.05% | −0.05% | 0.09% | −0.04% |
| Tyr | 0.50% | 0.05% | 0.26% | 0.39% | 0.43% | −0.05% | 0.34% | −0.01% |
| Val | 1.40% | 0.14% | 0.41% | 0.77% | 0.98% | −0.14% | 0.70% | −0.05% |

| | Δ%(Soy-vitellogenin-2) | | | | | |
|---|---|---|---|---|---|---|
| Vitellogenin-2 | Glycinin | Glycinin | Glycinin | Glycinin | Kunitz- | |
| AA | 3 | 4 | 5 | 7 | Trypsin | Lectin |
| Ala | −0.25% | 0.22% | −0.22% | −0.32% | −0.20% | 0.54% |
| Arg | −0.43% | 0.31% | −0.36% | −0.50% | −0.43% | 0.16% |
| Asn | −0.24% | 0.40% | −0.18% | −0.32% | −0.27% | 0.34% |
| Asp | −0.24% | 0.44% | −0.17% | −0.28% | −0.16% | 0.85% |
| Cys | 0.03% | 0.18% | 0.04% | 0.00% | 0.04% | 0.33% |
| Gln | −0.03% | 1.01% | 0.07% | −0.14% | −0.06% | 0.19% |
| Glu | −0.28% | 0.87% | −0.16% | −0.37% | −0.28% | 0.56% |
| Gly | −0.16% | 0.62% | −0.05% | −0.23% | −0.09% | 0.90% |
| His | −0.59% | −0.26% | −0.52% | −0.60% | −0.59% | −0.40% |
| Ile | −0.04% | 0.38% | 0.00% | −0.09% | 0.03% | 0.97% |
| Leu | −0.06% | 0.87% | 0.04% | −0.14% | 0.02% | 1.12% |
| Lys | −0.65% | −0.08% | −0.60% | −0.69% | −0.60% | 0.17% |
| Met | −0.08% | −0.02% | −0.07% | −0.09% | −0.06% | 0.11% |
| Phe | 0.02% | 0.31% | 0.04% | −0.05% | 0.06% | 0.88% |
| Pro | −0.07% | 0.71% | 0.03% | −0.14% | −0.05% | 0.59% |
| Ser | −5.56% | −4.61% | −5.45% | −5.64% | −5.54% | −4.58% |
| Thr | −0.14% | 0.27% | −0.08% | −0.18% | −0.08% | 0.48% |
| Trp | −0.04% | 0.08% | −0.03% | −0.05% | −0.03% | 0.08% |
| Tyr | −0.03% | 0.29% | 0.02% | −0.05% | 0.00% | 0.28% |
| Val | −0.08% | 0.65% | 0.01% | −0.14% | 0.00% | 0.79% |

TABLE 75
Calculation of the amino acid difference between a chicken fibroblast growth factor
in expressed at 10% and the most abundant seed storage proteins in Soybean (Δ%).

| | | | Δ%(Soy-Fibroblast growth factor) | | | | | |
|---|---|---|---|---|---|---|---|---|
| | | | β- | β- | β- | | | |
| Fibroblast growth factor | | | conglycinin | conglycinin | conglycinin | Cysteine | Glycinin | Glycinin |
| AA | % | 10.00% | α | α′ | β | Protease | 1 | 2 |
| Ala | 8.90% | 0.89% | −0.32% | −0.10% | 0.03% | −0.88% | 0.13% | −0.77% |
| Arg | 7.00% | 0.70% | 0.28% | 0.50% | 0.54% | −0.70% | 0.25% | −0.60% |
| Asn | 3.80% | 0.38% | 0.46% | 0.76% | 0.94% | −0.38% | 0.92% | −0.24% |
| Asp | 5.10% | 0.51% | 0.06% | 0.28% | 0.33% | −0.50% | 0.09% | −0.45% |
| Cys | 1.90% | 0.19% | −0.09% | −0.04% | −0.15% | −0.19% | 0.20% | −0.15% |
| Gln | 2.50% | 0.25% | 0.73% | 1.30% | 1.07% | −0.25% | 1.43% | −0.07% |
| Glu | 5.10% | 0.51% | 1.10% | 1.86% | 0.97% | −0.50% | 0.93% | −0.38% |
| Gly | 11.40% | 1.14% | −0.61% | −0.26% | −0.38% | −1.13% | 0.12% | −1.02% |
| His | 1.30% | 0.13% | 0.03% | 0.51% | 0.19% | −0.13% | 0.15% | −0.12% |
| Ile | 3.20% | 0.32% | 0.31% | 0.50% | 0.72% | −0.32% | 0.59% | −0.24% |
| Leu | 8.90% | 0.89% | 0.21% | 0.52% | 0.96% | −0.88% | 0.41% | −0.76% |
| Lys | 8.90% | 0.89% | −0.16% | 0.37% | −0.05% | −0.88% | −0.01% | −0.82% |
| Met | 1.90% | 0.19% | −0.13% | −0.07% | −0.11% | −0.19% | 0.06% | −0.16% |
| Phe | 5.70% | 0.57% | 0.04% | 0.31% | 0.63% | −0.57% | 0.27% | −0.49% |
| Pro | 7.00% | 0.70% | 0.12% | 0.33% | 0.22% | −0.70% | 0.32% | −0.61% |
| Ser | 5.70% | 0.57% | 0.35% | 0.81% | 0.79% | −0.56% | 0.62% | −0.46% |
| Thr | 3.80% | 0.38% | −0.16% | −0.03% | 0.06% | −0.38% | 0.32% | −0.32% |
| Trp | 0.60% | 0.06% | −0.02% | 0.03% | −0.06% | −0.06% | 0.08% | −0.05% |
| Tyr | 3.80% | 0.38% | −0.07% | 0.06% | 0.10% | −0.38% | 0.01% | −0.34% |
| Val | 3.80% | 0.38% | 0.17% | 0.53% | 0.74% | −0.38% | 0.46% | −0.29% |

| | Δ%(Soy-Fibroblast growth factor) | | | | | |
|---|---|---|---|---|---|---|
| Fibroblast growth factor | Glycinin | Glycinin | Glycinin | Glycinin | Kunitz- | |
| AA | 3 | 4 | 5 | 7 | Trypsin | Lectin |
| Ala | −0.82% | −0.35% | −0.79% | −0.89% | −0.77% | −0.03% |
| Arg | −0.63% | 0.11% | −0.56% | −0.70% | −0.63% | −0.04% |
| Asn | −0.30% | 0.34% | −0.24% | −0.38% | −0.33% | 0.28% |
| Asp | −0.47% | 0.21% | −0.40% | −0.51% | −0.39% | 0.62% |
| Cys | −0.16% | −0.01% | −0.15% | −0.19% | −0.15% | 0.14% |
| Gln | −0.14% | 0.90% | −0.04% | −0.25% | −0.17% | 0.08% |
| Glu | −0.42% | 0.73% | −0.30% | −0.51% | −0.42% | 0.42% |
| Gly | −1.07% | −0.29% | −0.96% | −1.14% | −1.00% | −0.01% |
| His | −0.12% | 0.21% | −0.05% | −0.13% | −0.12% | 0.07% |
| Ile | −0.27% | 0.15% | −0.23% | −0.32% | −0.20% | 0.74% |
| Leu | −0.81% | 0.12% | −0.71% | −0.89% | −0.73% | 0.37% |
| Lys | −0.85% | −0.28% | −0.80% | −0.89% | −0.80% | −0.03% |
| Met | −0.18% | −0.12% | −0.17% | −0.19% | −0.16% | 0.01% |
| Phe | −0.50% | −0.21% | −0.48% | −0.57% | −0.46% | 0.36% |
| Pro | −0.63% | 0.15% | −0.53% | −0.70% | −0.61% | 0.03% |
| Ser | −0.49% | 0.46% | −0.38% | −0.57% | −0.47% | 0.49% |
| Thr | −0.34% | 0.07% | −0.28% | −0.38% | −0.28% | 0.28% |
| Trp | −0.05% | 0.07% | −0.04% | −0.06% | −0.04% | 0.07% |
| Tyr | −0.36% | −0.04% | −0.31% | −0.38% | −0.33% | −0.05% |
| Val | −0.32% | 0.41% | −0.23% | −0.38% | −0.24% | 0.55% |

TABLE 76
Exemplary Cassettes Encoding CRISPR Enzyme

| Construct | Cassette Detail | CRISPR Target | Target Protein |
|---|---|---|---|
| AR03-13 | Cas9(KO 6x Glycinin)(GmSeed5:sig5:OKC1- | 6 Glycinin gene | KCN |
| | T:nosT) | | |
| AR09-02 | Cas9(KO 4x BCon) | 4 Beta-Conglycinin | None |
| | | Genes | |
| AR14-38 | Cas9(KO 6x Glycinin)(GmSeed2:sig2:OBC- | 6 Glycinin gene | BC-BC-KCN-LG |
| | T3:OBC-T2:OKC1-T:OLG1:AtHSP | | |
| | T:AtUbi10T) | | |
| AR14-39 | Cas9(KO 6x Glycinin)(GmSeed2:sig2:OBC- | 6 Glycinin gene | BC-FM-aS1-FM- |
| | T4:FM:OaS1-T2:FM:OaS1-T:FM:OBC- | | aS1-FM-BC |
| | T2:AtHSP T:AtUbi10T) | | |
| AR14-40 | Cas9(KO 4x BCon)(GmSeed2:sig2:OBC- | 4 Beta-Conglycinin | BC-BC-KCN-LG |
| | T3:OBC-T2:OKC1-T:OLG1:AtHSP | Genes | |
| | T:AtUbi10T) | | |
| AR14-41 | Cas9(KO 4x BCon)(GmSeed2:sig2:OBC- | 4 Beta-Conglycinin | BC-FM-aS1-FM- |
| | T4:FM:OaS1-T2:FM:OaS1- | Genes | aS1-FM-BC |
| | T:FM:OBC-T2:AtHSP T:AtUbi10T) | | |
| AR14-42 | Cas9(KO KTI3)(GmSeed2:sig2:OBC- | Kunitz Tripsin | BC-BC-KCN-LG |
| | T3:OBC-T2:OKC1-T:OLG1:AtHSP | Inhibitor 3 | |
| | T:AtUbi10T) | | |
| AR14-43 | Cas9(KO KTI3)(GmSeed2:sig2:OBC- | Kunitz Tripsin | BC-FM-aS1-FM- |
| | T4:FM:OaS1-T2:FM:OaS1-T:FM:OBC- | Inhibitor 3 | aS1-FM-BC |
| | T2:AtHSP T:AtUbi10T) | | |
| AR14-44 | Cas9(KO 3x Lox)(GmSeed2:sig2:OBC- | 3 Lipoxygenase | BC-BC-KCN-LG |
| | T3:OBC-T2:OKC1-T:OLG1:AtHSP | genes | |
| | T:AtUbi10T) | | |
| AR14-45 | Cas9(KO 3x Lox)(GmSeed2:sig2:OBC- | 3 Lipoxygenase | BC-FM-aS1-FM- |
| | T4:FM:OaS1-T2:FM:OaS1-T:FM:OBC- | genes | aS1-FM-BC |
| | T2:AtHSP T:AtUbi10T) | | |

### Example 25: Post-Translational Modifications as a Shielding Mechanisms for Degradation

Co-expression with a kinase: phosphorylation

The enzyme Fam20C (serine kinase) is responsible, at least in part, for the phosphorylation of caseins. Both the calcium phosphate and hydrophobic interactions between casein molecules are involved in the stability of the micelle (Bauman et al, 2006). In some embodiments, by co-expression with a kinase targeting casein, the phosphorylation increases the stability of the protein and creates a shield against protein degradation, as shown in FIG. 11A-FIG. 11E.

Cassettes (combinations of promoter, signal peptide and terminator) have been built and different fusions were assembled with various transgenes of interest (e.g., milk proteins). The kinase (Fam20C) was co-expressed in a different cassette and part of the same binary vector (cointegration of cassette kinase and cassette GOI, see percent events accumulating the recombinant protein at Table 77.

TABLE 77
Co-expression with kinase (Fam20C) provides increased protein
accumulation when caseins are in a multimeric form

| | | | Percent of events | |
|---|---|---|---|---|
| | | | accumulating the | |
| | | total | recombinant protein | |
| | | events | at the concentration: | |
| | | analyzed | 0-2% TSP | >2% TSP |
| No | BC | 39 | 100 | 0 |
| Kinase | KCN-BC | 48 | 100 | 0 |
| | BC-KCN | 12 | 100 | 0 |
| | BC-aS1-KCN | 28 | 96.4 | 3.6 |
| Kinase | BC/Fam20C | 9 | 100 | 0 |
| | KCN- | 28 | 7.1 | 92.9 |
| | BC/Fam20C | | | |
| | KCN-BC- | 36 | 38.9 | 61.1 |
| | aS1/Fam20C | | | |

### Example 26: Beta-Casein Fusion Proteins in E. coli and Soy

A comparative study was completed to determine differences in fusion protein expression in e. coli bacteria vs soy. Either total soluble proteins or inclusion body proteins were extracted from E. coli or Soybean seeds. Total proteins were loaded on SDS-PAGE (total concentrations indicated on the figure). SDS-PAGE was performed according to manufacturer's instructions (Product #5678105 BioRad, Hercules, CA, USA) under denaturing and reducing conditions. For immunoblotting proteins separated by SDS-PAGE were transferred to a PVDF membrane using Trans-Blot® Turbo™ Midi PVDF Transfer Packs (Product #1704157 BioRad) according to manufacturer's guidelines. Membranes were blocked with 3% BSA in phosphate buffered saline with 0.5% Tween-20, reacted with antigen specific antibody and subsequently reacted with fluorescent goat anti rabbit IgG (Product #60871 BioRad, CA). Membranes were scanned according to manufacturer's instructions using the ChemiDoc MP Imaging System (BioRad, CA) and analyzed using ImageLab Version 6.0.1 Standard Edition (Bio-Rad Laboratories, Inc.).

### Results

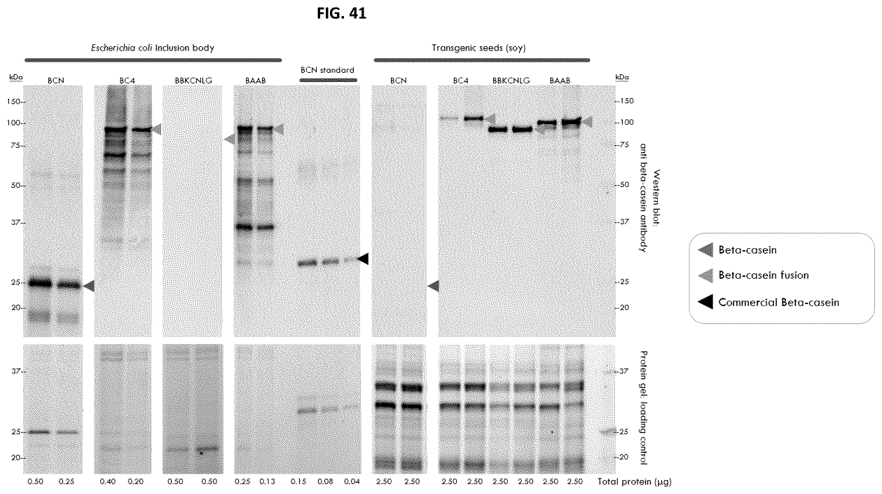

Beta-casein fusion proteins do not accumulate (BBKCNLG) or are highly degraded (BC4, BAAB) in E. coli in comparison to soy seeds, see FIG. 41.

### Example 27: Optimization of the Usage of Endogenous Transcription Factors (TFs)

As recombinant proteins are expressed under the control of seed-specific promoters, corresponding endogenous promoters can be knocked out to free any transcription factors (TFs) which naturally bind. The increased pool of free TFs become available to activate the exogenous promoter that drives the expression of the recombinant protein. In some cases, the endogenous promoter is knocked out in conjunction with part of the coding sequence. In this case, the endogenous protein won't be expressed. This creates an available pool of extra amino acids that can be used by the cell to synthesize the recombinant protein.

In brief, a minimum of about 500 bp of the Glycinin1 (GmSeed2) promoter sequence immediately upstream of the start codon is removed in conjunction with part of the Glycinin1 coding sequence (about 200 pb) via CRISPR-Cas9 technology, to achieve full endogenous promoter knockout and loss of expression of the glycinin 1 protein. Then, the recombinant protein is expressed behind the Glycinin 1 promoter or any promoter that shares the same pool of TFs. To introduce the recombinant protein in the same plant, three options are possible:

Option 1: Include GmSeed2 promoter knockout design and recombinant protein expressing cassette in the same T-DNA

Option 2: Create a GmSeed2 promoter knockout line that will be crossed with a stable line expressing recombinant protein.

Option 3: Create a GmSeed2 promoter knockout line that will be re-transformed with the construct containing the recombinant protein expressing cassette.

TABLE 78
Exemplary Cassettes for Knock Out of Endogenous Promoters

| Construct | Cassette Detail | CRISPR Target | Target Protein |
|---|---|---|---|
| AR14-21 | Cas9(KO_Gy1- | Glycinin1 | BC-BC-KCN-LG |
| | Pro1)(GmSeed12:coixss:OBC-T3:OBC- | promoter | |
| | T2:OKC1-T:OLG1:EUT:TM6) | | |
| AR14-22 | Cas9(KO_Gy1- | Glycinin1 | BC-BC-KCN-LG |
| | Pro2)(GmSeed12:coixss:OBC-T3:OBC- | promoter | |
| | T2:OKC1-T:OLG1:EUT:TM6) | | |
| AR14-23 | Cas9(KO_Gy1_g139a + g- | Glycinin1 | BC-BC-KCN-LG |
| | 849)(GmSeed12:coixss:OBC-T3:OBC- | promoter | |
| | T2:OKC1-T:OLG1:EUT:TM6) | | |
| AR14-24 | Cas9(KO_Gy1_g139a + g- | Glycinin1 | BC-BC-KCN-LG |
| | 697)(GmSeed12:coixss:OBC-T3:OBC- | promoter | |
| | T2:OKC1-T:OLG1:EUT:TM6) | | |
| AR14-25 | Cas9(KO_Gy1_g139a + g- | Glycinin1 | BC-BC-KCN-LG |
| | 679)(GmSeed12:coixss:OBC-T3:OBC- | promoter | |
| | T2:OKC1-T:OLG1:EUT:TM6) | | |
| AR14-26 | Cas9(KO_Gy1_g200 + g- | Glycinin1 | BC-BC-KCN-LG |
| | 849)(GmSeed12:coixss:OBC-T3:OBC- | promoter | |
| | T2:OKC1-T:OLG1:EUT:TM6) | | |
| AR14-27 | Cas9(KO_Gy1_g200 + g- | Glycinin1 | BC-BC-KCN-LG |
| | 697)(GmSeed12:coixss:OBC-T3:OBC- | promoter | |
| | T2:OKC1-T:OLG1:EUT:TM6) | | |
| AR14-28 | Cas9(KO_Gy1_g200 + g- | Glycinin1 | BC-BC-KCN-LG |
| | 679)(GmSeed12:coixss:OBC-T3:OBC- | promoter | |
| | T2:OKC1-T:OLG1:EUT:TM6) | | |
| AR14-46 | Cas9(KO Gy4 Pro)(GmSeed2:sig2:OBC- | Glycinin4 | BC-BC-KCN-LG |
| | T3:OBC-T2:OKC1-T:OLG1:AtHSP | promoter | |
| | T:AtUbi10T) | | |
| AR14-47 | Cas9(KO Gy4 Pro)(GmSeed2:sig2:OBC- | Glycinin4 | BC-FM-aS1-FM- |
| | T4:FM:OaS1-T2:FM:OaS1-T:FM:OBC- | promoter | aS1-FM-BC |
| | T2:AtHSP T:AtUbi10T) | | |
| AR14-48 | Cas9(KO BconA′ | Beta- | BC-BC-KCN-LG |
| | Pro)(GmSeed2:sig2:OBC-T3:OBC- | Conglycinin a′ | |
| | T2:OKC1-T:OLG1:AtHSP T:AtUbi10T) | subunit promoter | |
| AR14-49 | Cas9(KO BconA′ | Beta- | BC-FM-aS1-FM- |
| | Pro)(GmSeed2:sig2:OBC-T4:FM:OaS1- | Conglycinin a′ | aS1-FM-BC |
| | T2:FM:OaS1-T:FM:OBC-T2:AtHSP | subunit promoter | |
| | T:AtUbi10T) | | |
| AR14-50 | Cas9(KO KTI3 Pro)(GmSeed2:sig2:OBC- | Kunitz Tripsin | BC-BC-KCN-LG |
| | T3:OBC-T2:OKC1-T:OLG1:AtHSP | Inhibitor 3 | |
| | T:AtUbi10T) | Promoter | |
| AR14-51 | Cas9(KO KTI3 Pro)(GmSeed2:sig2:OBC- | Kunitz Tripsin | BC-FM-aS1-FM- |
| | T4:FM:OaS1-T2:FM:OaS1-T:FM:OBC- | Inhibitor 3 | aS1-FM-BC |
| | T2:AtHSP T:AtUbi10T) | Promoter | |

TABLE 79
Exemplary Cassettes
Cassette Details

(GmSeed12:coixss:OBC-T3:OBC-T2:OKC1-T:OLG1:EUT:TM6T)(7mu:ShCas9:NLS:nosT)
Cas9(KO_Gy1-Pro1)(GmSeed12:coixss:OBC-T3:OBC-T2:OKC1-T:OLG1:EUT:TM6T)
Cas9(KO_Gy1-Pro2)(GmSeed12:coixss:OBC-T3:OBC-T2:OKC1-T:OLG1:EUT:TM6T)
Cas9(KO_Gyl_g139a+g-849)(GmSeed12:coixss:OBC-T3:OBC-T2:OKC1-T:OLG1:EUT:TM6T)
Cas9(KO_Gyl_g139a+g-697)(GmSeed12:coixss:OBC-T3:OBC-T2:OKC1-T:OLG1:EUT:TM6T)
Cas9(KO_Gy1_g139a+g-679)(GmSeed12:coixss:OBC-T3:OBC-T2:OKC1-T:OLG1:EUT:TM6T)
Cas9(KO_Gy1_g200+g-849)(GmSeed12:coixss:OBC-T3:OBC-T2:OKC1-T:OLG1:EUT:TM6T)
Cas9(KO_Gy1_g200+g-697)(GmSeed12:coixss:OBC-T3:OBC-T2:OKC1-T:OLG1:EUT:TM6T)
Cas9(KO_Gyl_g200+g-679)(GmSeed12:coixss:OBC-T3:OBC-T2:OKC1-T:OLG1:EUT:TM6T)

### Example 28: Additional Food Compositions

The present disclosure also teaches edible compositions (i.e., food compositions) comprising recombinant mammalian milk protein, at specified ratios with a one or more secondary proteins. In some embodiments, the compositions comprise specified amounts of milk proteins in soy protein base. This example describes a non-limiting illustrative list compositions envisioned as part of this disclosure.

### Milk Substitute and or Coffee Creamer

It was hypothesized that recombinant milk proteins of the present disclosure could improve the organoleptic properties of milk substitute products. This hypothesis will be tested by producing one or more milk substitute products as follows.

The transgenic plants expressing the recombinant fusion proteins described herein are used to produce soy milk. Persons having skill in the art will be familiar with techniques for producing milk. For example, deskinned soybeans are placed in water and blended to produce a soybean mush. The blended mixture is strained to remove particulates, thereby producing a modified soy milk. The soy milk may be homogenized to reduce the droplet size of lipids. Additional lipids may be incorporated to a coffee creamer prior to homogenization. A control soy milk will (or can) be produced from wild type soybeans. The control and modified soy milks are optionally heated to kill pathogens/spoiling agents and increase shelf life.

The modified soy milk is expected to differ from the control soy milk in that the modified soy milk will contain higher ratios of milk protein to secondary proteins, including 7S globulin glycinin, 11S globulin, Lipoxygenase, and Kunitz Trypsin Inhibitor. These ratios will be measured using protein quantification techniques known in the art (e.g., Western Blot densitometry as described in Example 2).

The physical and organoleptic properties of the modified and control soy milks will be tested by methods known to those skilled in the art of food science. Viscosity, density, fat droplet size distribution, and emulsion stability will be tested.

In one illustrative test, the modified and control soy milks are added to separate cups of black coffee. The suitability of the soy milks as coffee creamers will be tested. For example, consistency of the soy milks in the coffee will be assessed visually. Particle size will be tested by methods known in the art, such as using a Malvern Mastersizer light scatter device. Taste tests will also be conducted comparing consumer preference of the modified or control soy milk compared to each other, and (optionally) compared to cow milk. It is expected that the modified soy milk will be a superior coffee creamer compared to the control soy milk.

In another illustrative test, the modified and control soy milks will be tested alone. For example, the modified and control soy milks will be tested in a consumer panel to assess its organoleptic properties and consumer preference. It is expected that the modified soy milk will be superior to traditional control soy milk.

### Cheese Substitute Compositions

It was hypothesized that recombinant milk proteins of the present disclosure could improve the organoleptic properties of spreadable cheese. This hypothesis will be tested by producing and testing soft cheeses as follows.

The transgenic plants expressing the recombinant fusion proteins described herein are used to produce soy milk. Persons having skill in the art will be familiar with techniques for producing milk. For example, deskinned soybeans are placed in water and blended to produce a soybean mush. The blended mixture is strained to remove particulates, thereby producing a modified soy milk. The soy milk may be homogenized to reduce the droplet size of lipids. Additional lipids may be incorporated prior to curdling. A control soy milk will produced from wild type soybeans. The next step is to make the spreadable cheese.

The control and modified soy milks are heated to boiling. Lemon juice or another acid is added to curdle the milk. The curdled mixture is sieved to collect particulates. The curdled particulates are then blended with optional seasonings (e.g., salt). The resulting product is a spreadable cheese made from the modified soymilk and a control spreadable cheese.

The modified spreadable cheese is expected to differ from the control cheese in that the modified spreadable cheese will contain higher ratios of milk protein to secondary proteins, including 7S globulin glycinin, 11S globulin, lipoxygenase, and kunitz trypsin inhibitor. These ratios will be measured using protein quantification techniques known in the art (e.g., Western Blot densitometry as described in Example 2).

The physical and organoleptic properties of the modified and control spreadable cheeses will be tested by methods known to those skilled in the art of food science. Density, rheology, thickness, spreadability, and stability will be tested. Particle size will be tested by methods known in the art, such as using a Malvern Mastersizer light scatter device. Protein content and protein quality will also be measured.

Taste tests will also be conducted comparing consumer preference of the modified or control spreadable cheeses., and (optionally) compared to spreadable cheeses made from cow milk. It is expected that the modified spreadable cheese will be superior in one or more properties compared to the control spreadable cheese made from traditional soy milk.

### Ice Cream Substitute Compositions

It was hypothesized that recombinant milk proteins of the present disclosure could improve the organoleptic properties of ice cream. This hypothesis will be tested by producing and testing ice cream as follows.

The transgenic plants expressing the recombinant fusion proteins described herein are used to produce soy milk. Persons having skill in the art will be familiar with techniques for producing milk. For example, deskinned soybeans are placed in water and blended to produce a soybean mush. The blended mixture is strained to remove particulates, thereby producing a modified soy milk. The soy milk is homogenized to reduce the droplet size of lipids. Additional lipids may be incorporated to a coffee creamer prior to homogenization. A control soy milk will be produced from wild type soybeans. The next step is to make the ice cream.

There are many recipes for producing ice cream. One example recipe is to separately mix the control and modified soy milks with egg yolks and (optionally) corn starch, sugar, and/or a flavoring. The mixtures are then heated and cooled in a mixer ice cream maker. The resulting product is an ice cream made from the modified soymilk and a control ice cream made from wild type control soy milk.

The modified ice cream is expected to differ from the control ice cream in that the modified spreadable cheese will contain higher ratios of milk protein to secondary proteins, including 7S globulin glycinin, 11S globulin, lipoxygenase, and kunitz trypsin inhibitor. These ratios will be measured using protein quantification techniques known in the art (e.g., Western Blot densitometry as described in Example 2).

The physical and organoleptic properties of the modified and control spreadable cheeses will be tested by methods known to those skilled in the art of food science. Density, thickness, flow, and stability will be tested. Particle size will be tested by methods known in the art, such as using a Malvern Mastersizer light scatter device. Protein content and protein quality will also be measured.

Taste tests will also be conducted comparing consumer preference of the modified or control ice cream. It is expected that the modified ice cream will be superior in one or more properties compared to the control ice cream made from traditional soy milk. Infant Formula Nutritional formulas

It was hypothesized that recombinant milk proteins of the present disclosure could improve the organoleptic properties of infant formula or other nutritional formula drinks. This hypothesis will be tested by producing and testing formulates and/or nutritional formulas. These products will be produced using standard recipes and the resulting products will be assess as done for other products in this example. Products made from modified soybean milk are expected to be superior to control products made from traditional soy milk. Experimental Products Recreated from Soy Milk and Cow Milk

The modified soy milks used above are expected to have the added benefit that they will lack allergens/irritants associated with cow milk. The modified soy milks do not require harvesting of milk from cows and is therefore expected to have additional food-safety benefits, and would also be considered cruelty-free.

Some of the organoleptic and flavor benefits described above may also be tested by producing soy milk+milk mixtures to replicate the expected benefits of the modified soy milk of the present disclosure.

### Example 29: Fatty Acid Profile Match

This disclosure teaches that altering fatty acid profiles of plant-based products can improve the properties and flavor of those products. This example demonstrates how plant-based products can be made to better mimic their animal-based counterparts through the fortification with short chain fatty acids.

The present disclosure teaches plants and plant-based products that better mimic animal products due to their expression of one or more milk proteins. This example demonstrates how these plant and plant-based products can be further enhanced through fatty acid profile matching.

Milkfats have diverse fatty acid profiles and are higher in short chain fatty acids (SCFA) than plant fats. (Markiewicz-Keszycka, M., Czyzak-Runowska, G., Lipinska, P., & Wójtowski, J. 2013. Fatty acid profile of milk—A review. Bulletin of the Veterinary Institute in Pulawy, 57(2), 135-139) In general, milk triacyglycerides also melt at body temperature. Palm oil has a similar melt point to milk fat, but two fatty acids, C16:0 and C18:1, represent 85% of the fatty acid profile and C18:2 contributes another 10%. Palm oil is used to replace milkfat in a number of applications including frozen desserts. While its texture is close to milkfat, it lacks the flavor release of butter. Coconut oil has a lower melt point than milkfat and is also used in some dairy alternatives. The melting profile of coconut oil is much steeper than milkfat and it does not release flavors as quickly as milkfat. The high level of C12:0 can impart a soapy flavor to products. Plant based oils (e.g. soy, sunflower, canola, etc.) have a high level of unsaturated fatty acids that impart a very low melting point and are susceptible to oxidative rancidity. These lipids are preferred for their low cost but are generally considered inferior to harder fats in most food applications. Hydrogenation is used to impart properties that are closer to animal fats. Modification of the fatty acid profile of soybean oil to impart properties of milkfat can be achieved by increasing the level of SCFA, increasing the level of saturated fats, and/or decreasing the level of unsaturation. An exemplary enhanced soybean oil is shown in Table 80, below.

TABLE 80
Fatty acid compositions of soybean oil, enhanced
soybean oil, palm oil, coconut oil, and butterfat
(fatty acids contributing <1% not shown)

| Fatty | Soybean | SCFA | Coconut | Palm | |
|---|---|---|---|---|---|
| acid | oil | soybean oil | oil | oil | Butterfat |
| C4:0 | — | 8.0% | — | — | 4.3% |
| C6:0 | — | 4.0% | — | — | 2.7% |
| C8:0 | — | 3.0% | 8.0% | — | 1.6% |
| C10:0 | — | — | 6.4% | — | 3.4% |
| C12:0 | — | — | 47.7% | — | 3.5% |
| C14:0 | — | — | 18.0% | 1.1% | 10.0% |
| C16:0 | 11.0% | 11.0% | 8.8% | 46.0% | 29.2% |
| C16:1 | — | — | — | — | 1.3% |
| C18:0 | 4.7% | 4.0% | 3.0% | 4.6% | 13.5% |
| C18:1 | 23.7% | 26.3% | 6.2% | 38.7% | 26.9% |
| C18:2 | 53.5% | 40.2% | 1.9% | 9.6% | 3.7% |
| C18:3 | 7.1% | 3.6% | — | — | — |

This example will blend lipids into various plant-based products to modulate the fatty acid profile to better match the properties of animal products. A soybean oil with high levels of SCFA combined with other lipid sources is expected to give an improved fat for use in dairy alternatives. The control and modified soy milks of Example 21 will be fortified with a blend of various plant-based oils/fats to mimic the fatty acid profile of milk and/or butter.

For example, the control and modified soy milks of Example 21 will be blended with about 50% soybean oil, 25% palm oil and 25% coconut oil. The resulting blend is expected to better mimic the fatty acid profile of animal butter. See Table 81, below.

TABLE 81
Fatty acid composition of butterfat vs. a blend of
50% SCFA soybean oil, 25% palm oil and 25% coconut
oil (fatty acids contributing <1% not shown).

| Fatty | | Soybean | SCFA soy + |
|---|---|---|---|
| acid | Butterfat | Oil | PO + CO |
| C4:0 | 4.30% | 0 | 4.00% |
| C6:0 | 2.70% | 0 | 2.20% |
| C8:0 | 1.60% | 0 | 3.50% |
| C10:0 | 3.40% | 0 | 1.60% |
| C12:0 | 3.50% | 0 | 11.80% |
| C14:0 | 10.00% | 0 | 4.70% |
| C16:0 | 29.20% | 11.00% | 19.20% |
| C16:1 | 1.30% | 0 | 0 |
| C18:0 | 13.50% | 4.70% | 3.90% |
| C18:1 | 26.90% | 23.70% | 24.40% |
| C18:2 | 3.70% | 53.50% | 23.00% |
| C18:3 | 0 | 7.10% | 1.80% |

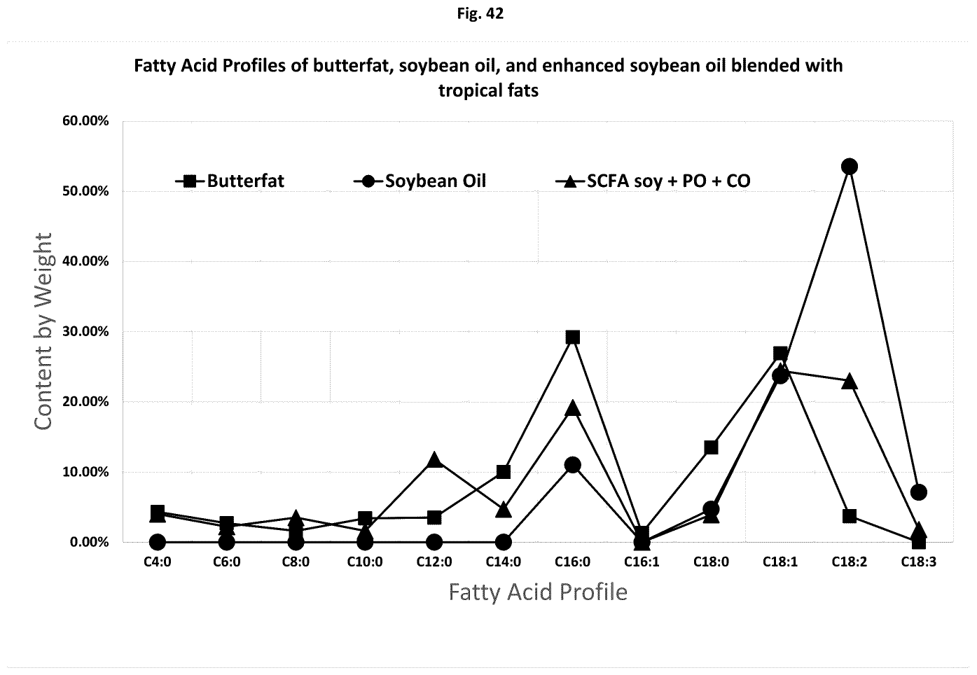

The expected fatty acid profile modulation is also presented visually in FIG. 42. Soybean oil fatty acid profiles differ, particularly with respect to longer chain fatty acids (Lee, J.-D., Bilyeu, K. D., & Shannon, J. G. 2007. Genetics and Breeding for Modified Fatty Acid Profile in Soybean Seed Oil. Journal of Crop Science and Biotechnology, 10(4), 201-210). The blend of 50% SCFA soybean oil, 25% palm oil and 25% coconut oil better matches that of butter.

Additional blends will be created, for example, using palm stearin, a fractionated palm oil higher in saturated fatty acids or by reducing the amount of SCFA soybean oil relative to harder fats. In some embodiments, this plant-based blend can replace butter in food compositions/recipes of the present disclosure.

The resulting products will be evaluated for differences in their physical properties and tastes. Specifically, the compositions of Example 28 will be fortified with various fatty acid blends to better match the profiles of animal products, including milk and butter. It is expected that fatty acid profile matched products will exhibit one or more properties superior to that of non-matched products.

### Example 30: Phosphorylation Patterns of Recombinant Proteins Expressed in Plants Vs Corresponding Proteins in a Bovine

The phosphorylation pattern of a beta-casein produced in plants versus a beta casein produced by bovine was analyzed using mass spectrometry after trypsin or chymotrypsin digestion. Two peptides were detected presenting a phosphorylation pattern in the bovine casein which was differed from the plant version.

For the peptide FQSEEQQQTEDELQDK (SEQ ID NO: 955), in the bovine, the ratio phosphorylated/non phosphorylated was 1 to 1 whereas in the plant recombinant protein the ratio was 1 to 10 suggesting very little phosphorylation in the plant recombinant protein.

For the peptide NVPGEIVESL (SEQ ID NO: 956), in the bovine, the ratio phosphorylated/non phosphorylated was 1 to 1 whereas in the plant recombinant protein the ratio was 1 to 2 suggesting less phosphorylation in the plant recombinant protein.

Prediction for phosphorylation in the bovine version.

>OBC-T2 (Bovine)
(SEQ ID NO: 913)
RELEELNVPGEIVESLSSSEESITRINKKIEKFQSEEQQQTEDELQDKI
HPFAQTQSLVYPFPGPIPNSLPQNIPPLTQTPVVVPPFLQPEVMGVSKV
KEAMAPKHKEMPFPKYPVEPFTESQSLTLTDVENLHLPLPLLQSWMHQP
HQPLPPTVMFPPQSVLSLSQSKVLPVPQKAVPYPQRDMPIQAFLLYQEP
VLGPVRGPFPIIV

S: Serine phosphorylation sites in bovine annotated on UniProt.

NVPGEIVESL (SEQ ID NO: 956) and FQSEEQQQTEDELQDK (SEQ ID NO: 955): peptides found in our study to be phosphorylated in bovine and to a lesser extent in plants.

### Example 31: Universal Vector Designs

Various universal vector designs are generated to increase expression of heterologous (e.g., bovine milk) proteins in plants. A universal vector can comprise a seed-specific promoter; a signal peptide; and a double terminator in an exemplary configuration. Alternate elements can be employed.

TABLE 82
Exemplary Universal Vector Designs
Exemplary Design
Seed-specific promoter:Signal Peptide(SP):GOI:terminator

| Exemplary | | | |
|---|---|---|---|
| promoter | Exemplary SP | GOI | Terminator |
| PvPhas, | GmSCB1, StPat21, | α-S1 casein, | AtHSP:AtUbi10, |
| BnNap, | 2Sss, Sig2, Sig12, | α-S2 casein, | EU:TM6 |
| AtOle1, | Sig8, Sig10, Sig11, | β-casein, | |
| GmSeed2, | and Coixss. | κ-casein, | |
| GmSeed3, | | para-κ-casein, | |
| GmSeed5, | | β-lactoglobulin, | |
| GmSeed6, | | α-lactalbumin, | |
| GmSeed7, | | lysozyme, | |
| GmSeed8, | | lactoferrin, | |
| GmSeed10, | | lactoperoxidase, | |
| GmSeed11, | | serum albumin, | |
| GmSeed12, | | and/or an | |
| pBCON, | | immunoglobulin. | |
| GmCEP1-L, | | | |
| GmTHIC, | | | |
| GmBg7S1, | | | |
| GmGRD, | | | |
| GmOLEA, | | | |
| GmOLER, | | | |
| Gm2S-1, and | | | |
| GmBBld-II | | | |

Exemplary designs are shown in Table 83 below and in FIG. 45A-FIG. 45D and FIG. 46A-FIG. 46G.

TABLE 83
Exemplary universal vectors
Universal design

| Plasmid ID | Cassette detail |
|---|---|
| AR15-143 | Rb7:GmSeed2:sig2:GFP:AtHSP T:AtUbi10T:Rb7 |
| AR15-255 | Rb7:GmSeed2:sig2:OBC-T5:OBC-T4:OBC-T3:OBC-T2:AtHSP T:AtUbi10T:Rb7 |
| AR15-256 | Rb7:GmSeed2:sig2:OBC-T3:OBC-T2:OKC1-T:OLG1:AtHSP T:AtUbi10T:Rb7 |
| AR15-448 | Rb7:GmSeed2:sig2:OBC-T5:OBC-T4:OBC-T3:OBC-T2:PFPS:AtHSP T:AtUbi10T:Rb7 |
| AR15-449 | Rb7:GmSeed2:sig2:OBC-T3:OBC-T2:OKC1-T:OLG1:PFPS:AtHSP T:AtUbi10T:Rb7 |
| AR15-478 | Rb7:GmSeed2:sig2:OBC-T4:OaS1-T2:OaS1-T:OBC-T2:PFPS:AtHSP T:AtUbi10T:Rb7 |
| AR15-479 | Rb7:GmSeed2:sig2:OKC1-T:OBC-T4:OBC-T3:OBC-T2:PFPS:AtHSP T:AtUbi10T:Rb7 |
| AR15-490 | Rb7:GmSeed2:sig2:OOVAL2 (intron 1):PFPS:AtHSP T:AtUbi10T:Rb7 |
| AR15-491 | Rb7:GmSeed2:sig2:OLG2 (intron 2):PFPS:AtHSP T:AtUbi10T:Rb7 |
| AR15-492 | Rb7:GmSeed2:sig2:GFP:PFPS:AtHSP T:AtUbi10T:Rb7 |
| AR15-493 | Rb7:GmSeed2:sig2:GmLBC2:PFPS:AtHSP T:AtUbi10T:Rb7 |
| AR15-494 | Rb7:GmSeed2:GmLBC2:AtHSP T:AtUbi10T:Rb7 |
| AR15-517 | Rb7:GmSeed2:sig2:OBC-T4:OaS1-T2:OaS1-T:OBC-T2:AtHSP T:AtUbi10T:Rb7 |
| AR15-518 | Rb7:GmSeed2:sig2:OKC1-T:OBC-T4:OBC-T3:OBC-T2:AtHSP T:AtUbi10T:Rb7 |
| AR15-519 | Rb7:GmSeed2:sig2:OOVAL2 (intron 1):AtHSP T:AtUbi10T:Rb7 |
| AR15-520 | Rb7:GmSeed2:sig2:OLG2 (intron 2):AtHSP T:AtUbi10T:Rb7 |
| AR15-521 | Rb7:GmSeed2:sig2:GmLBC2:AtHSP T:AtUbi10T:Rb7 |

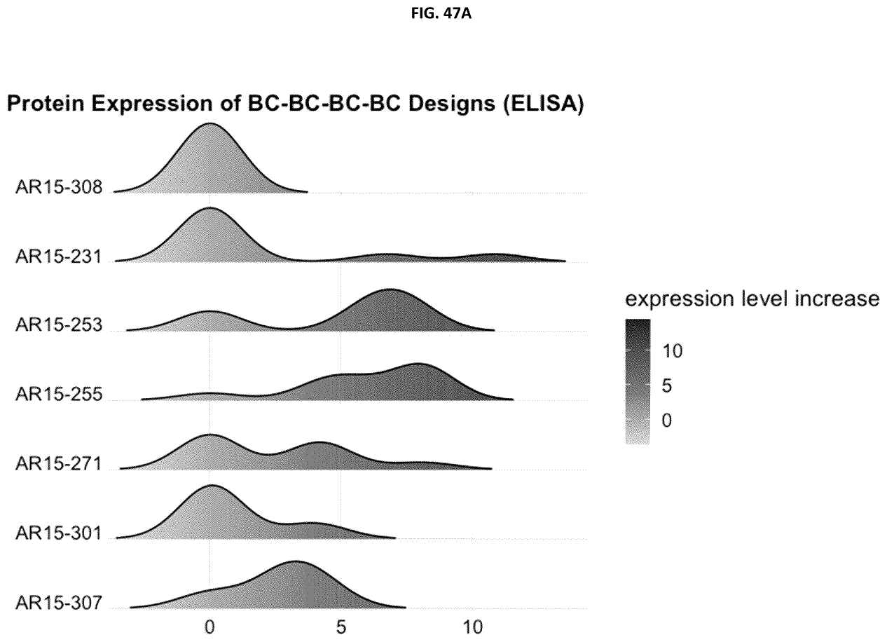

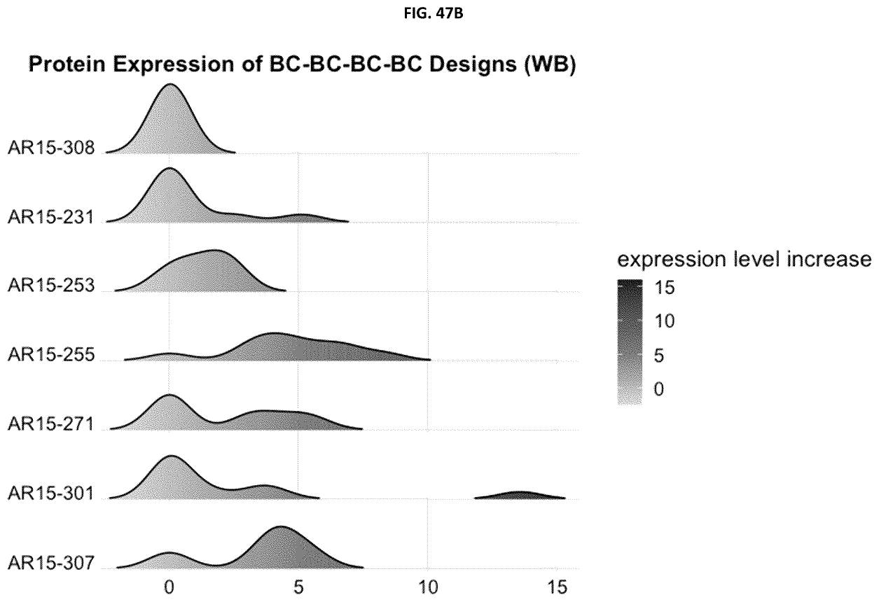

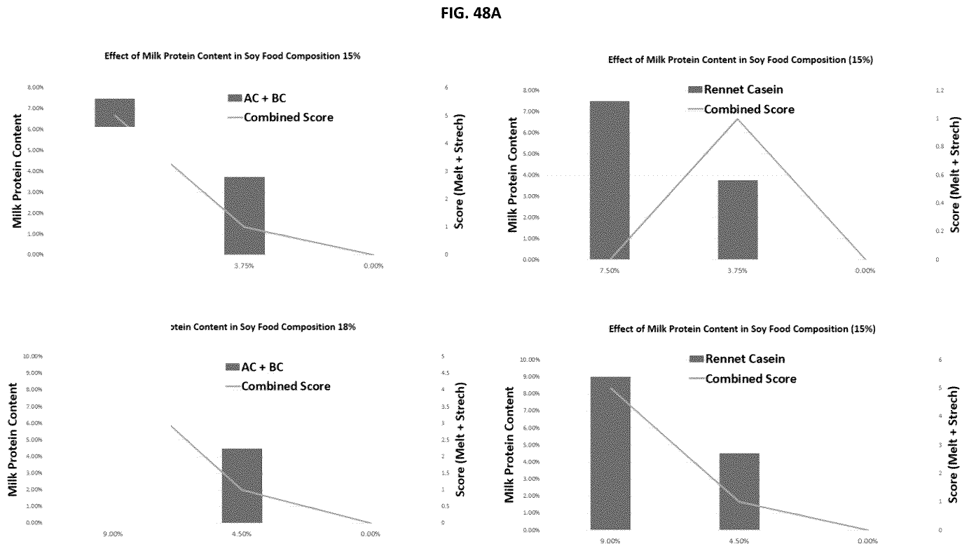

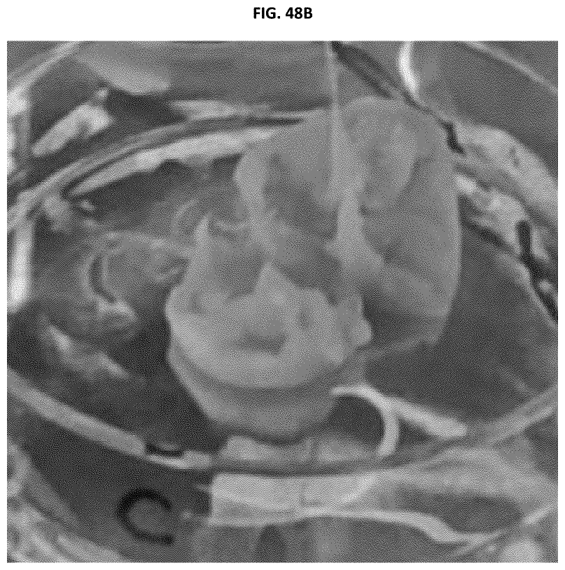

Plants were transformed with select exemplary constructs and protein expression was evaluated by ELISA, see Table 84 below and FIG. 47A (graphical representation of ELISA results for BC-BC-BC-BC constructs) and FIG. 47B (graphical representation of western blot results for BC-BC-BC-BC constructs).

TABLE 84
Protein Quantification by ELISA for BC-BC-BC-BC constructs

| | | | | Number of |
|---|---|---|---|---|
| Construct | Design | | p-value | Plants |
| AR15-308 | no sig2 | Control | | 10 |
| (FIG. 45A) | | | | |
| AR15-231 | sig2 | | 0.145 | 9 |
| (FIG. 45B) | | | | |
| AR15-253 | TM6 + sig2 | | 0.003 | 7 |
| (FIG. 45D) | | | | |
| AR15-255 | Rb7 + sig2 | | 0.0002 | 10 |
| (FIG. 45D) | | | | |
| AR15-271 | sig2 + KDEL | | 0.014 | 10 |
| (FIG. 45C) | | | | |
| AR15-301 | sig2 + ctVSD | | 0.062 | 9 |
| (FIG. 45C) | | | | |
| AR15-307 | sig2 + PFPS | | 0.0014 | 9 |
| (FIG. 45C) | | | | |

TABLE 85
Protein Quantification by Western
Blot for BC-BC-BC-BC constructs

| | | | | Number of |
|---|---|---|---|---|
| Construct | Design | | p-value | Plants |
| AR15-308 | no sig2 | Control | | 10 |
| AR15-231 | sig2 | | 1 | 9 |
| AR15-253 | TM6 + sig2 | | 0.023 | 7 |
| AR15-255 | Rb7 + sig2 | | 0.0007 | 10 |
| AR15-271 | sig2 + KDEL | | 0.146 | 10 |
| AR15-301 | sig2 + ctVSD | | 0.299 | 9 |
| AR15-307 | sig2 + PFPS | | 0.006 | 9 |

In a prophetic experiment, plants will be transformed with any configuration of the exemplary construct of Table 82. Protein expression will be evaluated by ELISA and Western Blot. Constructs yielding high protein expression will be selected.

### Example 32: Bovine Isolated Milk Proteins Improve Soy-Based Cheese Products

Cheese making has relied on dairy milk (e.g., bovine milk) as the major ingredient for over four thousand years. Dairy cheese is usually made from curds formed from dairy milk. Dairy milk can readily be made to form curds suitable for making cheese by contacting the dairy milk with rennet (an aspartic protease which cleaves kappa-casein) at mildly acidic pH. A variety of non-dairy cheese analogs are available that contain soy products (e.g., soymilk powder, soy protein, or soy flour), oils, and various thickeners. However, the taste and texture of such products does not match that of dairy-based cheeses. For example, soy “cheeses” are often curdled soy protein (i.e., tofu) that lacks the defining melt and stretch properties of true cheese.

To evaluate the effect of the use of bovine-isolated milk proteins on melt and stretch of cheese, various cheeses were produced utilizing protocols of the disclosure using different soy-to-casein ratios, ranging from no soy to 100% soy. Cheeses were generated according to the designs of Table 86.

TABLE 86
Experimental Designs

| | | | % Soy | | |
|---|---|---|---|---|---|
| | Source of | % Dairy | Protein | % Total | Ratio of |
| Sample ID | Dairy Protein | Protein | Isolate | Protein | Soy:Casein |
| C | AC + BC | 7.5 | 7.5 | 15 | 1:1 |
| D | AC + BC | 3.75 | 11.25 | 15 | 3:1 |
| E | None | 0 | 15 | 15 | 0:1 |
| H | Rennet Casein | 7.5 | 7.5 | 15 | 1:1 |
| I | Rennet Casein | 3.75 | 11.25 | 15 | 3:1 |
| L | AC + BC | 9 | 9 | 18 | 1:1 |
| M | AC + BC | 4.5 | 13.5 | 18 | 3:1 |
| Q | Rennet Casein | 9 | 9 | 18 | 1:1 |
| R | Rennet Casein | 4.5 | 13.5 | 18 | 3:1 |

The rennett 7.5% entry will repeated.

The cheeses of Table 86 were cooked according to the program schedule below and analyzed according to the parameters of Table 87.
Cheese premix 2 (00:00:00 Temp 50; 00:00:00 Speed 500; 00:05:00 End).
Cheese premix 3 (00:00:00 Temp 40; 00:00:00 Speed 200; 00:02:00 Speed 500; 00:05:00 End).
Cheese make 8-21 (00:00:00 Temp 95; 00:00:00 Speed 960; 00:01:00 Speed 500; 00:01:00 Temp 90; 00:02:00 Temp 85; 00:03:00 End).

TABLE 87
Properties of Cheese Compositions

| | | Premix #3 | | Cheese make 8-21 | | | |
|---|---|---|---|---|---|---|---|
| | pH | Time | Viscosity | Time | Viscosity | Trend | Comments |
| C | 5.66 | 5:00 | 504 | 0:46 | 3380 | decreasing | smooth, still pretty stretchy |
| D | 5.96 | 5:00 | 1696 | 1:25 | 3153 | decreasing | smooth, very little stretch |
| E | 5.64 | 5:00 | 1903 | 1:11 | 680 | decreasing | pasty, not stretchy |
| H | 5.36 | 2:30 | 290 | 1:12 | 391 | decreasing | same as A |
| I | 5.58 | 2:30 | 580 | 1:14 | 2149 | decreasing | smooth, stringy |
| L | 5.72 | 5:00 | 1319 | 0:44 | 5494 | increasing | smooth, v. sl. stretchy |
| M | 6.02 | 5:00 | 3607 | 1:17 | 4348 | increasing | pasty, no stretch |
| Q | 5.37 | 5:00 | 1357 | 0:41 | 5714 | increasing | smooth, somewhat stretchy |
| R | 5.51 | 5:00 | 2696 | 0:52 | 6396 | increasing | pasty, fluffy, not stretchy |

Cheese melt and stretch was determined after a 6 minute incubation at 450° F. according to the scoring mechanism whereby cheese firmness is assigned a score of 0 (Very Soft), 1 (slightly soft), 2 (soft), 3 (semi-firm), 4 (firm), and 5 (very firm). Melt score was determined as provided in Table 88. A composite score was also calculated by taking the sum of the firmness and melt of each representative cheese.

TABLE 88
Melt Quantification Metric

| % Pan Coverage | Melt Score |
|---|---|
| No change | 0 |
| 0-25 | 1 |
| 25-50 | 2 |
| 50-75 | 3 |
| >75 | 4 |

### Results

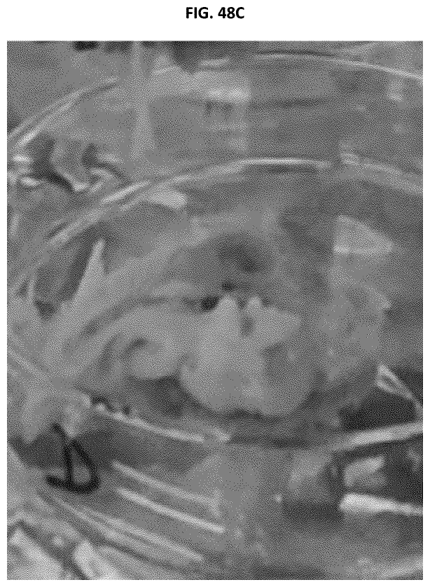

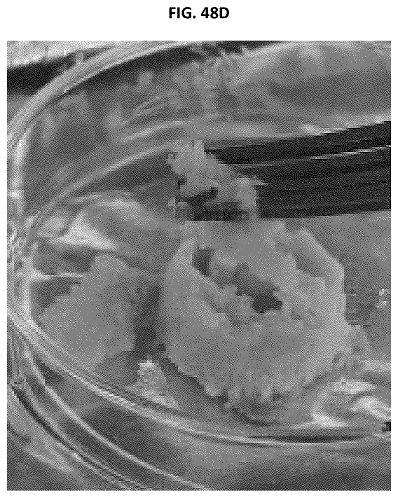

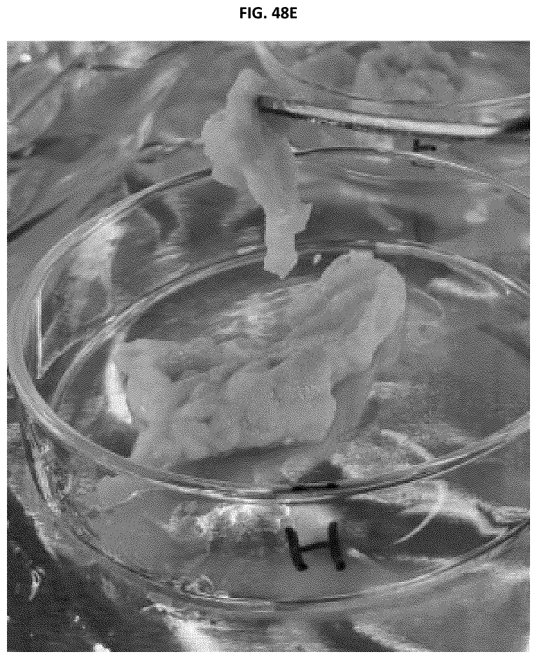

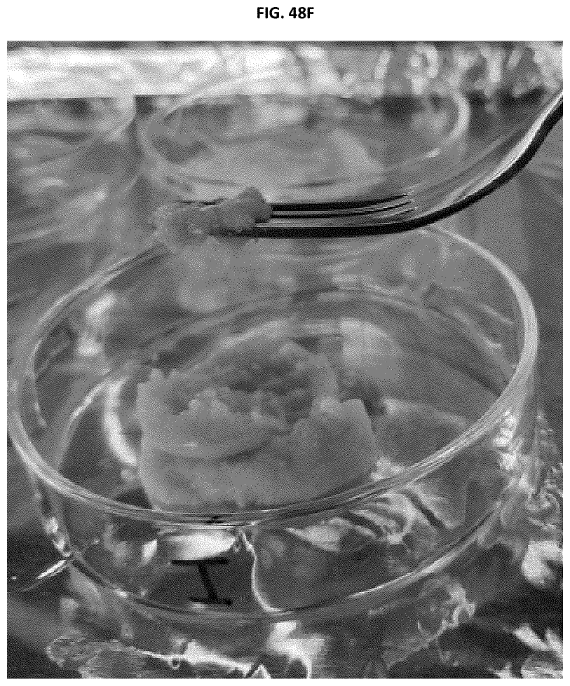

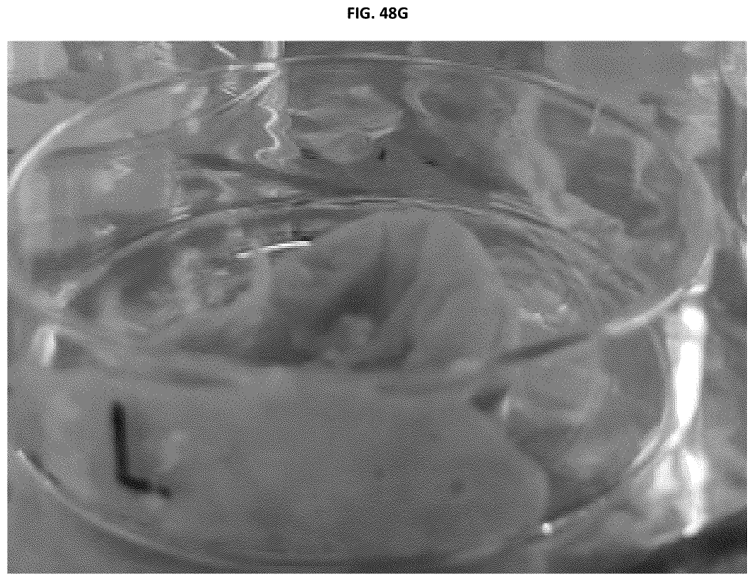

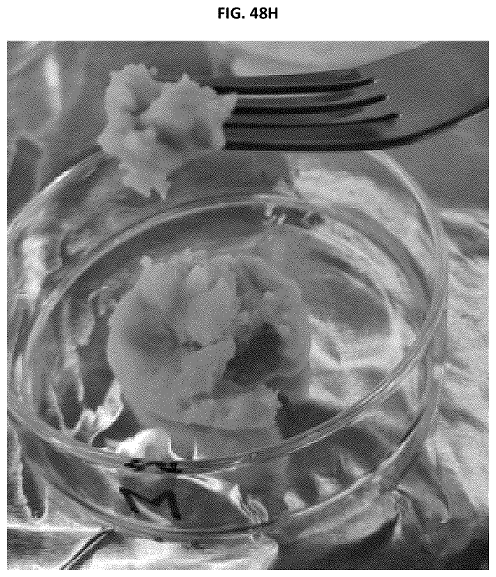

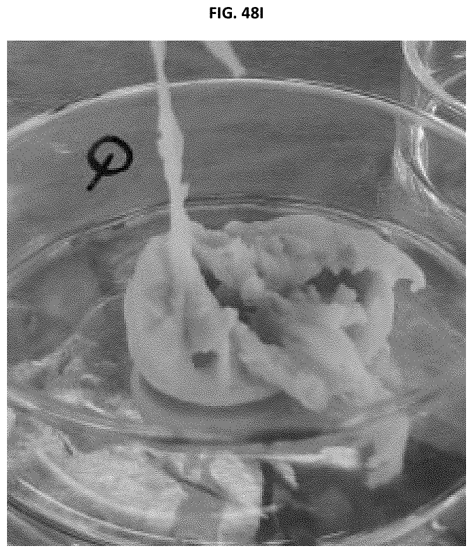

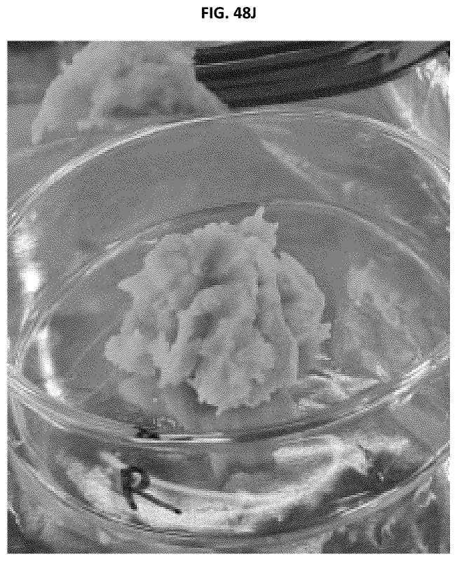

Sample cheeses were analyzed for firmness and melt, results are provided in Table 89 below. Representative images are shown in FIG. 48B-FIG. 48J.

TABLE 89
Cheese Firmness and Melt

| | Melt/stretch comments (450° F., 6 minutes) | | | |
|---|---|---|---|---|
| ID | Melt Score | Stretch | Comments | Firmness Score |
| C | 2 | 3 cm | firm, springy | 4 |
| D | 1 | 0 cm | firm, springy | 4 |
| E | 0 | 0 cm | softer, not springy | 3 |
| H | 0 | 0 cm | slightly less firm, springy | 3 |
| I | 1 | 0 cm | softer, not springy | 2 |
| L | 2.5 | 2 cm | firm, springy | 4 |
| M | 1 | 0 cm | firm, springy | 4 |
| Q | 1 | 4 cm | very firm, not springy | 5 |
| R | 1 | 0 cm | slightly less firm, springy | 3 |

Results show that the addition of even the smallest amount of casein imparts the food composition with melt and stretch properties of cheese. As shown in FIG. 48A, the 100% soy cheese has no melt or stretch. When bovine isolated casein is added, the melt and stretch composite score increases, thereby resulting in a plant-based cheese product that more closely resembles natural dairy cheese.

### Example 33: Cheese Food Compositions

Various cheese food products can be manufactured utilizing the compositions of the disclosure. Using vector constructs encoding only a subset of milk proteins, as described in the aforementioned examples, a subset of milk proteins have been used to generate completely novel cheeses. The novel cheeses are made with only a subset of milk proteins (e.g., with any of the combinations provided herein), those milk proteins having been expressed from dicotyledonous plants (e.g., soybean).

### Mozzarella-Style Cheese

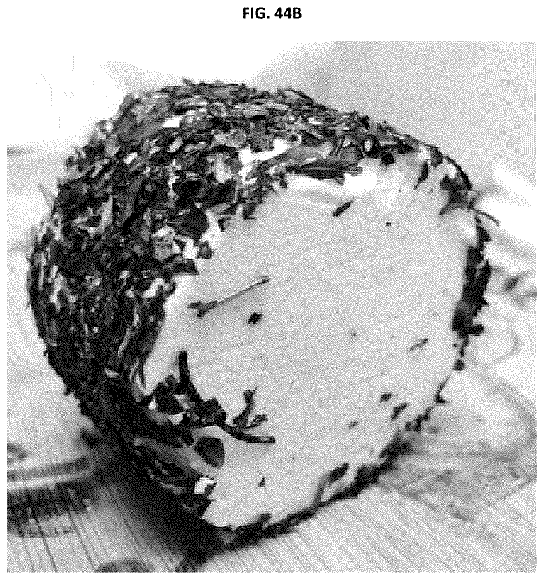

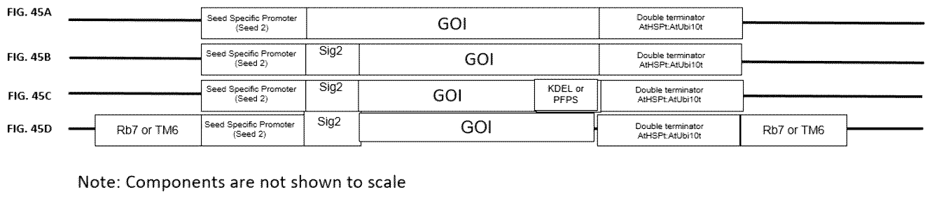

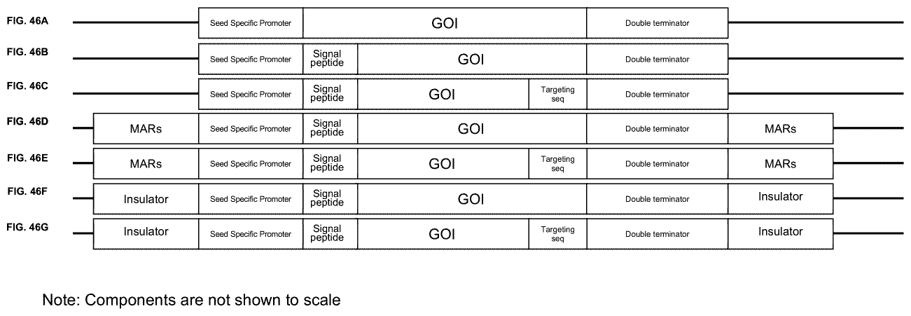

A mozzarella-style cheese was generated using soymilk extracted from harvested soybeans from a κ-casein expressing soybean plan. The soymilk was mixed with plant fats, sugars, chymosin, and cultures to form a cheese curd. The curd was then separated from the liquid phase, and was evaluated for its sensory properties. The resulting mozzarella-type cheese exhibited melt and stretch similar to that of animal cheeses, while only comprising the transgenically expressed κ-casein and no other casein proteins. An image of the mozzarella cheese is provided at FIG. 44A.

### Soft Cheese

A soft cheese was also produced from soymilk of the κ-casein expressing soybean plant. Soymilk containing plant-produced κ-casein was mixed with plant fats, sugars, chymosin, and cultures to form a cheese curd. The resulting soft cheese exhibited similar flavor and texture to that of animal soft cheese, despite only comprising transgenically expressed κ-casein and no other casein proteins. An image of the soft cheese is provided at FIG. 44B.

### Example 34: Soybean Varieties Amenable to Recombinant Milk Protein Production

The inventors will select a soybean variety from Tables 23-31 and perform one or more of the various genomic architecture alterations of the present disclosure to the variety. These genetic edits/genomic modifications will create robust biofactories to produce recombinant proteins.

In one experiment, the inventors will perform genomic modifications to enable the soybean varieties to produce recombinant milk proteins. The genetically modified soybean seed will comprise at least one of the following genetic modifications: a recombinant DNA construct encoding a fusion protein comprising at least one milk protein; a recombinant DNA construct encoding a protein capable of forming a protein body; a recombinant DNA construct encoding a prolamin; a first recombinant DNA construct encoding a milk protein and a second recombinant DNA construct encoding a prolamin; a recombinant DNA construct encoding a milk protein that has been modified to have an amino acid sequence different from the native animal expressed milk protein; a recombinant DNA construct encoding a milk protein that has been modified to promote addition of a post-translational modification; a recombinant DNA construct encoding a milk protein that has been modified to prevent addition of a post-translational modification; a recombinant DNA construct encoding an enzyme that alters post-translational modification of protein; a recombinant DNA construct encoding an enzyme capable of modifying a protein; a recombinant DNA construct encoding a kinase; or a genetic modification that modulates the expression of a plant protease.

The inventors expect the genetically modified soybean biofactories to produce significant recombinant milk protein per acre of planted soybean, as detailed in Example 21.

### Milk Protein Sequences

The following Table 90 describes various representative species of milk proteins exemplified in the disclosure.

TABLE 90
Milk Protein Sequences of the Disclosure

| | | | |
|---|---|---|---|
| SEQ | | | Accession |
| ID NO | Description | Genus/species | Number |

Kappa casein sequences

| | | | |
|---|---|---|---|
| 3 | Optimized kappa-casein | Artificial (codon optimized | |
| | truncated version 1 | Bos taurus) | |
| | (OKC1-T) | | |
| 4 | Optimized kappa-casein | Bos taurus | |
| | truncated version 1 | | |
| | (OKC1-T) | | |
| 85 | Kappa casein | Capra hircus | |
| 86 | Kappa casein | Ovis aries | |
| 87 | Kappa casein | Bubalus bubalis | |
| 88 | Kappa casein | Camelus dromedaries | |
| 89 | Kappa casein | Camelus bactrianus | |
| 90 | Kappa casein | Bos mutus | |
| 91 | Kappa casein | Equus caballus | |
| 92 | Kappa casein | Equus asinus | |
| 93 | Kappa casein | Rangifer tarandus | |
| 94 | Kappa casein | Alces alces | |
| 95 | Kappa casein | Vicugna pacos | |
| 96 | Kappa casein | Bos indicus | |
| 97 | Kappa casein | Lama glama | |
| 98 | Kappa casein | Homo sapiens | |
| 148 | Kappa casein | Bos taurus | NP_776719.1 |
| 149 | | | AAI02121.1 |
| 150 | | | AAA30433.1 |
| 151 | | | AAB26704.1 |
| 152 | | | 1406275A |
| 153 | | | AAF72097.1 |
| 154 | | | AAD32139.1 |
| 155 | | | XP_024848756.1 |
| 156 | | | CAF03625.1 |
| 157 | | | ABN42697.1 |
| 158 | | | AAD32140.1 |
| 159 | | | ALC76014.1 |
| 160 | | | DAA28589.1 |
| 161 | | | ADT82665.1 |
| 162 | | | ADT82666.1 |
| 163 | | | CAH56573.1 |
| 164 | | | ADT82669.1 |
| 165 | Kappa casein | Capra hircus | QIZ03342.1 |
| 166 | | | AYN74373.1 |
| 167 | | | AAM12026.1 |
| 168 | | | AFZ92921.1 |
| 169 | | | NP_001272516.1 |
| 170 | | | AAM12027.1 |
| 171 | | | AAR06605.1 |
| 172 | | | AAL90873.1 |
| 173 | | | AFZ92919.1 |
| 174 | | | QIZ03345.1 |
| 175 | | | AAR91623.1 |
| 176 | | | AAK17010.1 |
| 177 | | | AAL93193.1 |
| 178 | | | AFZ92918.1 |
| 179 | | | AAL90872.1 |
| 180 | | | AFZ92917.1 |
| 181 | | | AA039432.1 |
| 182 | | | AAL90871.1 |
| 183 | | | AAO39431.1 |
| 184 | Kappa casein | Ovis aries | NP_001009378.1 |
| 185 | | | AAP69943.1 |
| 186 | Kappa casein | Bubalus bubalis | NP_001277901.1 |
| 187 | | | AXE74388.1 |
| 188 | | | APQ30586.1 |
| 189 | | | AXE74385.1 |
| 190 | | | XP_006071184.1 |
| 191 | | | AXE74386.1 |
| 192 | Kappa casein | Bos mutus | XP_005897104.1 |
| 193 | | | XP_014334109.1 |
| 194 | | | MXQ92034.1 |
| 195 | Kappa casein | Bos indicus | XP_019818432.1 |
| 196 | | | ACF15188.1 |
| 197 | | | ACF15186.1 |
| 198 | | | ACF15190.1 |
| 199 | | | ABY81250.1 |
| 200 | | | ABY81251.1 |
| 20 | | | ADT82668.1 |
| 202 | | | ADT82663.1 |
| 203 | | | ADT82671.1 |
| 204 | | | ADT82670.1 |
| 205 | | | AAQ73171.1 |
| 206 | Kappa casein | Jeotgalicoccus coquinae | WP_188357548.1 |
| 207 | (Hypothetical Protein) | | WP_188357549.1 |
| 208 | Kappa casein isoform X1 | Bison bison bison | XP_010837415.1 |
| 209 | | | XP_010837416.1 |
| 210 | Kappa casein | Bos grunniens | AFM93768.1 |
| 211 | | | AXE74296.1 |
| 212 | | | AAM25910.1 |
| 213 | | | ABU53615.1 |
| 214 | | | AAM25909.1 |
| 215 | | | AAF63191.1 |
| 216 | Kappa casein | Bos indicus × Bos taurus | AAF72096.1 |
| 217 | | | AAF72098.1 |
| 218 | Kappa casein (precursor) | Oreamnos americanus | P50423.1 |
| 219 | Kappa casein (precursor) | Naemorhedus goral | P50422.1 |
| 220 | Kappa casein | Odocoileus virginianus texanus | XP_020729185.1 |
| 221 | Kappa casein (precursor) | Capricornis sumatraensis | P50420.1 |
| 222 | Kappa casein (precursor) | Capricornis crispus | BAA03287.1 |
| 223 | | | P42156.1 |
| 224 | Kappa casein (precursor) | Capricornis swinhoei | P50421.1 |
| 225 | Kappa casein (precursor) | Saiga tatarica | P50425.1 |
| 226 | Kappa casein (precursor) | Rupicapra rupicapra | P50424.1 |
| 227 | Kappa casein (precursor) | Cervus nippon | P42157.1 |
| 228 | Kappa casein | Bos frontalis | ADF58295.1 |
| 229 | Kappa casein | Muntiacus reevesi | KAB0354473.1 |
| | (hypothetical protein | | |
| | FD755_023011) | | |
| 230 | Kappa casein | Muntiacus muntjak | KAB0341224.1 |
| | (hypothetical protein | | |
| | FD754_018150) | | |
| 231 | Kappa casein | Madoqua saltiana | AFY03578.1 |
| 232 | Kappa casein | Gazella dorcas | AFY03574.1 |
| 233 | Kappa casein | Gazella arabica | AFY03576.1 |
| 234 | Kappa casein | Capra ibex ibex | AAP80529.1 |
| 235 | Kappa casein | Ovis ammon severtzovi | ADB66396.1 |
| 236 | Kappa casein | Ovis orientalis gmelini | ADB66423.1 |
| 237 | | | ADB66420.1 |
| 238 | Kappa casein | Cervus hanglu yarkandensis | KAF4013038.1 |
| | (hypothetical protein | | |
| | G4228_004474) | | |
| 239 | Kappa casein | Procapra gutturosa | AFY03581.1 |
| 240 | | | AFY03580.1 |
| 1 | Optimized para-kappa- | Artificial (codon optimized | |
| | casein truncated version | Bos taurus) | |
| | 1 (paraOKC1-T) | | |
| 2 | Optimized para-kappa- | Bos taurus | |
| | casein truncated version | | |
| | 1 (paraOKC1-T) | | |
| 241 | Kappa casein isoform X1 | Bos taurus | AAA30433.1 |
| 242 | | | 1406275A |
| 243 | | | AAI02121.1 |
| 244 | | | NP_776719.1 |
| 245 | | | DAA28589.1 |
| 246 | | | AAB26704.1 |
| 247 | | | XP_024848756.1 |
| 248 | | | ABN42697.1 |
| 249 | | | AAF72097.1 |
| 250 | | | 721588A |
| 25 | | | AAD32139.1 |
| 252 | | | AAD32140.1 |
| 253 | | | CAF03625.1 |
| 254 | Kappa casein | Jeotgalicoccus coquinae | WP_188357548.1 |
| 255 | (hypothetical protein) | | WP_188357549.1 |
| 256 | Kappa casein isoform X1 | Bos mutus | XP_005897104.1 |
| 257 | | | XP_014334109.1 |
| 258 | | | MXQ92034.1 |
| 259 | Kappa casein | Bos indicus | XP_019818432.1 |
| 260 | | | ACF15188.1 |
| 261 | | | ABY81250.1 |
| 262 | | | ABY81251.1 |
| 263 | | | ACF15186.1 |
| 264 | | | ACF15190.1 |
| 265 | | | ADT82668.1 |
| 266 | Kappa casein | Bos grunniens | AXE74296.1 |
| 267 | | | AFM93768.1 |
| 268 | | | AAM25910.1 |
| 269 | | | AAM25909.1 |
| 270 | | | ABU53615.1 |
| 271 | Kappa casein isoform X1 | Bison bison bison | XP_010837415.1 |
| 272 | | | XP_010837416.1 |
| 273 | Kappa casein (precursor) | Bubalus bubalis | NP_001277901.1 |
| 274 | | | XP_006071184.1 |
| 275 | | | AXE74388.1 |
| 276 | | | AXE74385.1 |
| 277 | | | APQ30586.1 |
| 278 | | | AXE74386.1 |
| 279 | Kappa casein (precursor) | Oreamnos americanus | P50423.1 |
| 280 | Kappa casein (precursor) | Capricornis swinhoei | P50421.1 |
| 281 | Kappa casein (precursor) | Naemorhedus goral | P50422.1 |
| 282 | Kappa casein (precursor) | Capricornis sumatraensis | P50420.1 |
| 283 | Kappa casein (precursor) | Capricornis crispus | BAA03287.1 |
| 284 | | | P42156.1 |
| 285 | Kappa casein (precursor) | Saiga tatarica | P50425.1 |
| 286 | Kappa casein | Bos indicus × Bos taurus | AAF72096.1 |
| 287 | | | AAF72098.1 |
| 288 | Kappa casein (precursor) | Capra hircus | NP_001272516.1 |
| 289 | | | AYN74373.1 |
| 290 | | | QIZ03345.1 |
| 291 | | | QIZ03342.1 |
| 292 | | | AFZ92921.1 |
| 293 | | | AAR06605.1 |
| 294 | | | AAM12026.1 |
| 295 | | | AAL93193.1 |
| 296 | | | AAR91623.1 |
| 297 | | | AFZ92917.1 |
| 298 | | | AAM12027.1 |
| 299 | | | AAL90873.1 |
| 300 | | | AFZ92918.1 |
| 301 | | | AAL90871.1 |
| 302 | | | AAL90872.1 |
| 303 | | | AAL31535.1 |
| 304 | | | AAL31534.1 |
| 305 | | | ABK59545.1 |
| 306 | | | AAO39432.1 |
| 307 | | | AFZ92919.1 |
| 308 | | | AAK17010.1 |
| 309 | | | AAO39431.1 |
| 310 | | | AAP80475.1 |
| 311 | Kappa casein | Odocoileus virginianus texanus | XP_020729185.1 |
| 312 | Kappa casein (precursor) | Rupicapra rupicapra | P50424.1 |
| 313 | Kappa casein (precursor) | Ovis aries | NP_001009378.1 |
| 314 | | | AAP69943.1 |
| 315 | Kappa casein (precursor) | Cervus nippon | P42157.1 |
| 316 | Kappa casein | Gazella arabica | AFY03576.1 |
| 317 | Kappa casein | Muntiacus muntjak | KAB0341224.1 |
| | (hypothetical protein | | |
| | FD754_018150) | | |
| 318 | Kappa casein | Muntiacus reevesi | KAB0354473.1 |
| | (hypothetical protein | | |
| | FD755_023011) | | |
| 319 | Kappa casein | Gazella dorcas | AFY03575.1 |
| 320 | Kappa casein | Procapra gutturosa | AFY03581.1 |
| 321 | | | AFY03580.1 |
| 322 | Kappa casein | Madoqua saltiana | AFY03578.1 |
| 323 | Kappa casein | Ammotragus lervia | QIN85723.1 |
| 324 | | | QIN85720.1 |
| 325 | | | QIN85721.1 |
| 326 | Kappa casein | Capra sibirica | AAP80568.1 |
| 327 | Kappa casein | Ovis canadensis canadensis | ADB66397.1 |
| 328 | | | ADB66402.1 |
| 329 | Kappa casein | Gazella subgutturosa marica | AFY03577.1 |
| 330 | Kappa casein | Antilope cervicapra | AFY03573.1 |
| 331 | Kappa casein | Capra ibex ibex | AAP80529.1 |
| 332 | Kappa casein | Ovis vignei arkal | ADB66436.1 |
| 333 | | | ADB66442.1 |
| 334 | Kappa casein | Ovis ammon collium | ADB66395.1 |
| 335 | Kappa casein | Ovis vignei blanfordi | ADB66445.1 |
| 336 | Kappa casein | Ovis orientalis gmelini | ADB66423.1 |
| 337 | | | ADB66420.1 |
| 338 | Kappa casein | Ovis orientalis × vignei | ADB66465.1 |
| 339 | Kappa casein | Ovis vignei vignei | ADB66456.1 |
| 340 | Kappa casein | Ovis ammon severtzovi | ADB66396.1 |

Alpha S1 casein sequences

| | | | |
|---|---|---|---|
| 7 | Optimized alpha S1- | Artificial (codon optimized | |
| | casein truncated version | Bos taurus) | |
| | 1(OaS1-T) | | |
| 8 | Optimized alpha S1- | Bos taurus | |
| | casein truncated version | | |
| | 1(OaS1-T) | | |
| 99 | Alpha S1 casein | Capra hircus | |
| 100 | Alpha S1 casein | Ovis aries | |
| 101 | Alpha S1 casein | Bubalus bubalis | |
| 102 | Alpha S1 casein | Camelus dromedaries | |
| 103 | Alpha S1 casein | Camelus bactrianus | |
| 104 | Alpha S1 casein | Bos mutus | |
| 105 | Alpha S1 casein | Equus caballus | |
| 106 | Alpha S1 casein | Equus asinus | |
| 107 | Alpha S1 casein | Bos indicus | |
| 108 | Alpha S1 casein | Lama glama | |
| 109 | Alpha S1 casein | Homo sapiens | |
| 341 | Alpha S1 casein | Bos taurus | ABW98943.1 |
| 342 | | | XP_024848771.1 |
| 343 | | | ABW98940.1 |
| 344 | | | ACG63494.1 |
| 345 | | | XP_015327132.1 |
| 346 | | | XP_024848772.1 |
| 347 | | | 1308122A |
| 348 | | | ABW98949.1 |
| 349 | | | AAA30429.1 |
| 350 | | | XP_015327135.1 |
| 351 | | | XP_015327134.1 |
| 352 | | | XP_024848773.1 |
| 353 | | | XP_015327133.1 |
| 354 | | | XP_024848774.1 |
| 355 | | | XP_015327136.1 |
| 356 | | | XP_024848775.1 |
| 357 | | | XP_005208084.1 |
| 358 | | | XP_024848776.1 |
| 359 | | | XP_015327137.1 |
| 360 | | | XP_015327138.1 |
| 361 | | | XP_024848777.1 |
| 362 | | | XP_024848778.1 |
| 363 | | | XP_015327139.1 |
| 364 | | | ABW98944.1 |
| 365 | | | XP_015327140.1 |
| 366 | | | XP_024848779.1 |
| 367 | | | XP_015327141.1 |
| 368 | | | XP_024848780.1 |
| 369 | | | XP_015327142.1 |
| 370 | | | ABW98945.1 |
| 371 | | | XP_024848782.1 |
| 372 | | | ABW98951.1 |
| 373 | | | XP_024848784.1 |
| 374 | | | XP_024848783.1 |
| 375 | | | ABW98950.1 |
| 376 | | | ABW98941.1 |
| 377 | | | XP_005208086.1 |
| 378 | | | ABW98942.1 |
| 379 | | | ABW98937.1 |
| 380 | | | ABW98952.1 |
| 381 | | | ABW98954.1 |
| 382 | | | ABW98953.1 |
| 383 | | | ABW98955.1 |
| 384 | | | ABW98957.1 |
| 385 | Alpha S1 casein | Capra hircus | XP_017904616.1 |
| 386 | | | QIZ03312.1 |
| 387 | | | ALJ30147.1 |
| 388 | | | P18626.2 |
| 389 | | | XP_017904617.1 |
| 390 | | | AFN44013.1 |
| 391 | | | QIZ03319.1 |
| 392 | | | CAA51022.1 |
| 393 | | | NP_001272624.1 |
| 394 | | | ALJ30148.1 |
| 395 | | | QIZ03317.1 |
| 396 | | | QIZ03310.1 |
| 397 | | | QIZ03318.1 |
| 398 | | | XP_017904618.1 |
| 399 | | | XP_017904620.1 |
| 400 | | | XP_017904619.1 |
| 401 | | | XP_017904621.1 |
| 402 | | | XP_017904622.1 |
| 403 | Alpha S1 casein | Ovis aries | XP_012034747.1 |
| 404 | | | P04653.3 |
| 405 | | | AAB34797.1 |
| 406 | | | ACJ46472.1 |
| 407 | | | XP_027826521.1 |
| 408 | | | XP_027826520.1 |
| 409 | | | ACR58469.1 |
| 410 | | | ACJ46473.1 |
| 411 | | | AAB34798.1 |
| 412 | | | NP_001009795.1 |
| 413 | Alpha S1 casein | Bubalus bubalis | AAZ14098.1 |
| 414 | | | APQ30583.1 |
| 415 | | | 062823.2 |
| 416 | | | XP_006071187.1 |
| 417 | | | QCP57314.1 |
| 418 | | | XP_025145744.1 |
| 419 | | | QPO15022.1 |
| 420 | | | XP_025145745.1 |
| 421 | | | ACJ14317.1 |
| 422 | | | XP_006071188.1 |
| 423 | | | XP_025145747.1 |
| 424 | | | XP_025145746.1 |
| 425 | | | XP_025145748.1 |
| 426 | | | XP_025145749.1 |
| 427 | | | XP_025145750.1 |
| 428 | | | XP_025145751.1 |
| 429 | | | XP_025145752.1 |
| 430 | | | XP_025145753.1 |
| 431 | Alpha S1 casein | Bos mutus | XP_005902100.1 |
| 432 | Alpha S1 casein | Bos indicus | XP_019818428.1 |
| 433 | Alpha S1 casein | Jeotgalicoccus coquinae | WP_188357546.1 |
| 434 | (hypothetical protein) | | GGE26809.1 |
| 435 | Alpha S1 casein | Bison bison bison | XP_010850445.1 |
| 436 | Alpha S1 casein | Bos grunniens | AXE74293.1 |
| 437 | Alpha S1 casein | Jeotgalicoccus aerolatus | WP_188349304.1 |
| 438 | (hypothetical protein) | | WP_188352531.1 |
| 439 | Alpha S1 casein | Muntiacus muntjak | KAB0341228.1 |
| | (hypothetical protein | | |
| | FD754_018154) | | |
| 440 | Alpha S1 casein | Muntiacus reevesi | KAB0354470.1 |
| | (hypothetical protein | | |
| | FD755_023008) | | |

Alpha S2 casein sequences

| | | | |
|---|---|---|---|
| 83 | Optimized alpha S2- | Artificial (codon optimized | |
| | casein truncated version | Bos taurus) | |
| | 1(OaS2-T) | | |
| 84 | Optimized alpha S2- | Bos taurus | |
| | casein truncated version | | |
| | 1(OaS2-T) | | |
| 110 | Alpha S2 casein | Capra hircus | |
| 111 | Alpha S2 casein | Ovis aries | |
| 112 | Alpha S2 casein | Bubalus bubalis | |
| 113 | Alpha S2 casein | Camelus dromedaries | |
| 114 | Alpha S2 casein | Camelus bactrianus | |
| 115 | Alpha S2 casein | Bos mutus | |
| 116 | Alpha S2 casein | Equus caballus | |
| 117 | Alpha S2 casein | Equus asinus | |
| 118 | Alpha S2 casein | Vicugna pacos | |
| 119 | Alpha S2 casein | Bos indicus | |
| 120 | Alpha S2 casein | Lama glama | |
| 441 | Alpha S2 casein | Bos taurus | AAI14774.1 |
| 442 | | | XP_024848786.1 |
| 443 | | | XP_015327143.1 |
| 444 | Alpha S2 casein | Capra hircus | QIS93310.1 |
| 445 | | | NP_001272514.1 |
| 446 | | | CAB94236.1 |
| 447 | | | QIS93322.1 |
| 448 | | | AAB32166.1 |
| 449 | | | QIS93306.1 |
| 450 | | | XP_013820127.2 |
| 451 | | | QIS93323.1 |
| 452 | | | QIZ03322.1 |
| 453 | | | QIS93316.1 |
| 454 | | | CAB59920.1 |
| 455 | | | CAC21704.2 |
| 456 | | | QIS93307.1 |
| 457 | | | XP_013820130.2 |
| 458 | | | QIS93319.1 |
| 459 | | | QIS93321.1 |
| 460 | | | XP_013820128.2 |
| 46 | | | QIS93304.1 |
| 462 | | | XP_013820129.2 |
| 463 | | | QIS93305.1 |
| 464 | | | QIS93314.1 |
| 465 | | | QIS93317.1 |
| 466 | | | XP_013820132.2 |
| 467 | | | XP_013820131.2 |
| 468 | Alpha S2 casein | Ovis aries | ADB65931.1 |
| 469 | | | NP_001009363.1 |
| 470 | | | ADB65933.1 |
| 471 | | | ADB65935.1 |
| 472 | | | ADB65934.1 |
| 473 | | | ADB65932.1 |
| 474 | Alpha S2 casein | Bubalus bubalis | NP_001277794.1 |
| 475 | | | AAZ80050.1 |
| 476 | | | CAA06534.2 |
| 477 | | | AFB69498.1 |
| 478 | | | XP_006071185.2 |
| 479 | | | AAZ57423.1 |
| 480 | | | APQ30584.1 |
| 481 | | | XP_025145302.1 |
| 482 | | | XP_025145301.1 |
| 483 | Alpha S2 casein | Bos mutus | XP_014335716.1 |
| 484 | | | ELR51813.1 |
| 485 | Alpha S2 casein | Jeotgalicoccus aerolatus | WP_188352530.1 |
| 486 | (hypothetical protein) | | GGE08804.1 |
| 487 | Alpha S2 casein | Jeotgalicoccus coquinae | WP_188357545.1 |
| | (hypothetical protein) | | |
| 488 | Alpha S2 casein | Bos grunniens | AXE74294.1 |
| 489 | Alpha S2 casein | Bison bison bison | XP_010850447.1 |
| 490 | Alpha S2 casein | Bos indicus × Bos taurus | XP_027401112.1 |
| 491 | Alpha S2 casein | Odocoileus virginianus texanus | XP_020729187.1 |
| 492 | Alpha S2 casein | Muntiacus muntjak | KAB0341229.1 |
| | (hypothetical protein | | |
| | FD754_018155) | | |
| 493 | Alpha S2 casein | Muntiacus reevesi | KAB0354254.1 |
| | (hypothetical protein | | |
| | FD755_022792) | | |
| 494 | Alpha S2 casein | Cervus elaphus | OWK13818.1 |
| | (CSN1S2) | hippelaphus | |

Beta-casein sequences

| | | | |
|---|---|---|---|
| 5 | Optimized beta-casein | Artificial (codon optimized | |
| | truncated version 2 | Bos taurus) | |
| | (OBC-T2) | | |
| 6 | Optimized beta-casein | Bos taurus | |
| | truncated version 2 | | |
| | (OBC-T2) | | |
| 121 | Beta casein | Capra hircus | |
| 122 | Beta casein | Ovis aries | |
| 123 | Beta casein | Bubalus bubalis | |
| 124 | Beta casein | Camelus dromedaries | |
| 125 | Beta casein | Camelus bactrianus | |
| 126 | Beta casein | Bos mutus | |
| 127 | Beta casein | Equus caballus | |
| 128 | Beta casein | Equus asinus | |
| 129 | Beta casein | Alces alces | |
| 130 | Beta casein | Vicugna pacos | |
| 131 | Beta casein | Bos indicus | |
| 132 | Beta casein | Lama glama | |
| 133 | Beta casein | Homo sapiens | |
| 495 | Beta casein | Bos taurus | AAB29137.1 |
| 496 | | | AAA30431.1 |
| 497 | | | 1314242A |
| 498 | | | AGT56763.1 |
| 499 | | | AAI11173.1 |
| 500 | | | XP_010804480.2 |
| 501 | | | AAA30430.1 |
| 502 | | | XP_015327157.2 |
| 503 | | | ABR10906.1 |
| 504 | | | ABL74247.1 |
| 505 | | | QCI03091.1 |
| 506 | | | QCI03090.1 |
| 507 | | | CAC37028.1 |
| 508 | Beta casein | Capra hircus | P33048.1 |
| 509 | | | QIZ03333.1 |
| 510 | | | CAB39200.1 |
| 511 | | | AAK97639.1 |
| 512 | | | XP_005681778.2 |
| 513 | | | QLI42602.1 |
| 514 | | | XP_013820153.1 |
| 515 | | | QLI42606.1 |
| 516 | | | QHN12643.1 |
| 517 | | | ABQ52487.1 |
| 518 | | | QHN12642.1 |
| 519 | | | CAB39313.1 |
| 520 | | | QHN12644.1 |
| 521 | | | AWN06750.1 |
| 522 | Beta casein | Ovis aries | P11839.3 |
| 523 | | | NP_001009373.1 |
| 524 | Beta casein | Bubalus bubalis | QHB80269.1 |
| 525 | | | APQ30585.1 |
| 526 | | | QHB80272.1 |
| 527 | | | QHB80273.1 |
| 528 | | | NP_001277808.1 |
| 529 | | | Q9TSI0.1 |
| 530 | | | XP_006071186.1 |
| 531 | | | CAA06535.1 |
| 532 | | | 1004269A |
| 533 | | | ADD31643.1 |
| 534 | | | ADD31644.1 |
| 535 | | | AAT09469.1 |
| 536 | | | ABL10285.1 |
| 537 | | | ABA41625.1 |
| 538 | | | ABA41623.1 |
| 539 | Beta casein | Bos mutus | MXQ92033.1 |
| 540 | | | XP_014335713.1 |
| 541 | | | XP_005902099.2 |
| 542 | | | XP_014335715.1 |
| 543 | | | XP_014335714.1 |
| 544 | Beta casein | Bos indicus | AQY78354.1 |
| 545 | | | AQY78355.1 |
| 546 | | | ABL75279.1 |
| 547 | | | ABY27644.1 |
| 548 | | | AWN06759.1 |
| 549 | | | AGZ84117.1 |
| 550 | Beta casein | Bison bison bison | XP_010850446.1 |
| 551 | Beta casein (hypothetical | Jeotgalicoccus aerolatus | WP_188352529.1 |
| | protein) | | |
| 552 | Beta casein (hypothetical | Jeotgalicoccus coquinae | WP_188357544.1 |
| | protein) | | |
| 553 | Beta casein (precursor) | Bos indicus × Bos taurus | ARU83745.1 |
| 554 | | | AWN06757.1 |
| 555 | | | AWN06758.1 |
| 556 | Beta casein | Bos grunniens | AXE74295.1 |
| 557 | | | AEY63644.1 |
| 558 | | | AEY63645.1 |
| 559 | | | AEC13563.1 |
| 560 | Beta casein | Neophocaena asiaeorientalis | XP_024597374.1 |
| | | asiaeorientalis | |
| 561 | Beta casein | Odocoileus virginianus texanus | XP_020729180.1 |
| 562 | Beta casein (hypothetical | Muntiacus reevesi | KAB0354325.1 |
| | protein FD755_022863) | | |
| 563 | Beta casein (hypothetical | Muntiacus muntjak | KAB0345505.1 |
| | protein FD754_022431) | | |

Beta-Lactoglobulin sequences

| | | | |
|---|---|---|---|
| 9 | Optimized Beta | Artificial (codon optimized | |
| | Lactoglobulin 1 (OLG1) | Bos taurus) | |
| 10 | Optimized Beta | Bos taurus | |
| | Lactoglobulin 1 (OLG1) | | |
| 11 | Optimized Beta | Artificial (codon optimized | |
| | Lactoglobulin 2 (OLG2) | Bos taurus) | |
| 12 | Optimized Beta | Artificial (codon optimized | |
| | Lactoglobulin 3 (OLG3) | Bos taurus) | |
| 13 | Optimized Beta | Artificial (codon optimized | |
| | Lactoglobulin 4 (OLG4) | Bos taurus) | |
| 564 | Beta Lactoglobulin | Bos taurus | 5K06_A |
| 565 | | | 1B0O_A |
| 566 | | | NP_776354.2 |
| 567 | | | 3PH5_A |
| 568 | | | 1BEB_A |
| 569 | | | 6QPD_A |
| 570 | | | 6Q17_A |
| 571 | | | DAA24277.1 |
| 572 | | | 5HTD_A |
| 573 | | | 6QPE_A |
| 574 | | | 6RWR_A |
| 575 | | | 1BSO_A |
| 576 | | | 6RWQ_A |
| 577 | | | ACG59280.1 |
| 578 | | | 5NUJ_A |
| 579 | | | 5NUM_A |
| 580 | | | 1UZ2_X |
| 581 | | | CAA32835.1 |
| 582 | | | 1CJ5_A |
| 583 | | | 5NUK_A |
| 584 | | | 5NUN_A |
| 585 | | | 732164A |
| 586 | | | XP_024854027.1 |
| 587 | | | AAA30411.1 |
| 588 | Beta Lactoglobulin | Capra hircus | 40MW_A |
| 589 | | | NP_001272468.1 |
| 590 | | | ABQ51182.1 |
| 591 | Beta Lactoglobulin | Ovis aries | 4NLI_A |
| 592 | | | NP_001009366.1 |
| 593 | | | 4CK4_A |
| 594 | | | 4CK4_B |
| 595 | Beta Lactoglobulin | Bubalus bubalis | 0601265A |
| 596 | | | P02755.2 |
| 597 | | | NP_001277893.1 |
| 598 | | | QOQ34530.1 |
| 599 | | | APQ30587.1 |
| 600 | | | ABG78270.1 |
| 601 | Beta Lactoglobulin | Bos mutus | XP_005888577.1 |
| 602 | | | MXQ94840.1 |
| 603 | Beta Lactoglobulin | Bos indicus | XP_019826641.1 |
| 604 | Beta Lactoglobulin | Jeotgalicoccus coquinae | WP_188357550.1 |
| | (lipocalin/fatty-acid | | |
| | binding family protein) | | |
| 605 | Beta Lactoglobulin | Jeotgalicoccus schoeneichii | WP_188349305.1 |
| | (lipocalin/fatty-acid | | |
| | binding family protei | | |
| 606 | Beta Lactoglobulin | Bison bison bison | XP_010855058.1 |
| 607 | Beta Lactoglobulin | Ovis sp. | AAA31510.1 |
| 608 | Beta Lactoglobulin | Ovis aries musimon | P67975.1 |
| 609 | Beta Lactoglobulin | Odocoileus virginianus texanus | XP_020744123.1 |
| 610 | Beta Lactoglobulin, | Rangifer tarandus | 1YUP_A |
| | Chain A | | |
| 611 | Beta Lactoglobulin | Rangifer tarandus tarandus | AAZ57420.1 |
| 612 | Beta Lactoglobulin | Muntiacus muntjak | KAB0364864.1 |
| | (hypothetical protein | | |
| | FD754_009020) | | |
| 613 | Beta Lactoglobulin | Muntiacus reevesi | KAB0379658.1 |
| | (hypothetical protein | | |
| | FD755_007442) | | |
| 614 | Beta Lactoglobulin, | Equus caballus | 3KZA_A |
| | Chain A | | |

TABLE 91
Additional Exemplary Glycotags

| | | | | No. of | | | | | |
|---|---|---|---|---|---|---|---|---|---|
| | | | | Hyp- | | | | | |
| | | | | containing | | | | | |
| | | | | peptide | 1KP groups with | | | | |
| | | | | hits in | Hyp-containing | | | | |
| 1KP group | | Glyco- | Hyp containing | 1KP | peptide hits | Example 1KP sequences with | 1KP | | Reference |
| and species | Protein | sylationa | motifsb,c | MAABd | in 1KP MAAB | peptide matches (MAAB Class)e | group | Species | sf |
| Green algae | AGP | | | | | | | | |
| Volvox | Chimeric | Ara: | IYPSVGSSSIVTPSW | | | | | | Godl et |
| carteri | AGP | Gal 1:1g | TAIGGSTLPLPLONA | | | | | | al. |
| | (Pherophorin) | | OOSOLO (SEQ ID | | | | | | 1997 |
| | | | NO: 957) | | | | | | |
| Moss | AGP | | | | | | | | |
| Physco- | P. patens | AG | XVOOOAGSOFQOVO | | | >YEPO Locus_1020 | Moss | Physco- | Lee et |
| mitrela | AGP2 | (Yariv) | S (SEQ ID | | | (Class 1, GPI-AGP)j | | mitrium | al. |
| patens | | | NO: 958) | | | MASSNTMSVAAALCLVTLFASVIVQVAAQEP | | sp. | 2005 |
| | | | | | | ASAPTA | | | |
| | | | | | | TPPVGAPTVPPPAGSPFQPPTSSPPAASGPA | | | |
| | | | | | | SVPPPPSPS | | | |
| | | | | | | AETPTATDSPPAAEGPGPATTPSSDAFTVAP | | | |
| | | | | | | TVSIVLMST | | | |
| | | | | | | LAYLLY (SEQ ID NO: 959) | | | |
| Physco- | P. patens | AG | SAGOVMAVVOOVTOO | | | | | | |
| mitrela | AGP | (Yariv) | VTOSV (SEQ ID | | | | | | |
| | | | NO: 960) | | | | | | |
| patens | | | SOASSAGOOMGSOOS | | | | | | |
| | | | OAOA (SEQ ID | | | | | | |
| | | | NO: 961) | | | | | | |
| | | | SOLTKAEVFYELKDL | | | | | | |
| | | | KGY (SEQ ID | | | | | | |
| | | | NO: 962) | | | | | | |
| Conifers | AGP | | | | | | | | |
| Pinus taeda | Classical | AG | ASEAOLSSAQAOKSA | 1 | Conifers | >MFTM_Locus_6686 | Conifers | Pinus | Loopstra |
| | AGP, | (Yariv) | LRHRA (SEQ ID | | | (Class 4, non-GPI-AGP) | | jeffreyi | et |
| | PtaAGP3 | | NO: 963) | | | METSTRSAVLLSLIAAAMMIHCCLADHQQYK | | | al. 2000 |
| | | | | | | VDESHE | | | |
| | | | | | | QWGFLTNNKAWSYILASEAPLSSAQAPKSAL | | | |
| | | | | | | RHRAKAS | | | |
| | | | | | | SAPVASSPSVSPSPTAAEAVPSASSTSESPA | | | |
| | | | | | | SSPATPPAQN | | | |
| | | | | | | GSPS (SEQ ID NO: 964) | | | |
| | Extensin | | | | | | | | |
| Pseudotsuga | Classical | Arah | KSOOOOXY (SEQ | 1450 | Monocots, | >AREG_Locus_407 | Conifers | Notho- | Fong et |
| | | | ID NO: 965) | | Cycadales, | (Class 2, CL-EXT) | | tsuga | al. |
| menziesii | extensin, | | LSOOOOYPAOAOYY | | Leptosporangiate | MALLAGMSFIRNLLLVLIVGCIYAANPSAAS | | longi- | 1992 |
| | SP2 | | (SEQ ID NO: 966) | | Monilophytes, | YYKSPPPK | | bracteata | |
| | | | AVOVSOOY (SEQ | 74 | Asterids, | PYPYKSPPPPSHVPSPVHYKPPPAPTPVPKP | | | |
| | | | ID NO: 967) | | Conifers, | PVVEKPPPK | | | |
| | | | ALPXYATVSXPXYPXG | | Lycophytes, | AVPISPPYKYKSPPPPKPIPAPYAKPPP | | | |
| | | | (SEQ ID NO: 968) | | Commelinids, | VHKPLPPPSPPKS | | | |
| | | | ASOOAXT (SEQ | | Rosids, | WPSPLPPHHKPSPVTKPPKATPSPPEKPYGY | | | |
| | | | ID NO: 969) | | Mosses, | KSPPPPAHV | | | |
| | | | KPOOAOTOVV (SEQ | 308 | Hornworts, | VSPAPPKAVTAFPPPHPKPPVYPSPPTHHYF | | | |
| | | | ID NO: 970) | | Core Eudicots, | YKSPPP | | | |
| | | | KSOOOTHT (SEQ | | Basal | PPY (SEQ ID NO: 972) | | | |
| | | | ID NO: 971) | | Eudicots, | | | | |
| | | | | | Magnoliids, | | | | |
| | | | | | Chloranthales, | | | | |
| | | | | | Eusporangiate | | | | |
| | | | | | Monilophytes, | | | | |
| | | | | | Basalmost | | | | |
| | | | | | angiosperms, | | | | |
| | | | | | Liverworts, | | | | |
| | | | | | Chromista | | | | |
| | | | | | Conifers, | | | | |
| | | | | | Liverworts | | | | |
| | | | | | Monocots, Core | | | | |
| | | | | | Eudicots, | | | | |
| | | | | | Asterids, | | | | |
| | | | | | Mosses, | | | | |
| | | | | | Conifers, | | | | |
| | | | | | Leptosporangiate | | | | |
| | | | | | Monilophytes, | | | | |
| | | | | | Basal | | | | |
| | | | | | Eudicots, | | | | |
| | | | | | Basalmost | | | | |
| | | | | | angiosperms, | | | | |
| | | | | | Chromista, | | | | |
| | | | | | Rosids, | | | | |
| | | | | | Commelinids, | | | | |
| | | | | | Lycophytes, | | | | |
| | | | | | Liverworts, | | | | |
| | | | | | Magnoliids, | | | | |
| | | | | | Green Algae, | | | | |
| | | | | | Gnetales, | | | | |
| | | | | | Cycadales | | | | |
| | Other | | | | | | | | |
| Pseudotsuga | Hybrid | Arah | KPOVOVIPPOVVKPO | | | | | | Kiel- |
| menziesii | HRGP, | | OVYKPOVOVIPP | | | | | | iszewski |
| | | | (SEQ ID NO: 973) | | | | | | et al. |
| | PHRGP | | OVVKPOVYKIP[P/O] | | | | | | 1992 |
| | | | VIKP (SEQ ID | | | | | | |
| | | | NO: 974) | | | | | | |
| Monocots/ | | | | | | | | | |
| Commelinids | AGP | | | | | | | | |
| Lollium | AGP | Arah | AEAOAOAOAS | 1 | Conifers | >AWQB Locus 369 | Conifers | Picea | Gleeson |
| multiflorum | | | (SEQ ID NO: 975) | | | (Class 1, GPI-AGP) | | engel- | et al. |
| | | | | | | MGFKCRLYFAILLWLCIQATCCRAHDRVLVR | | mannii | 1989 |
| | | | | | | DRVLPTTKT | | | |
| | | | | | | GIVGVALAAAKNATKSVTPVASPKPSETKPE | | | |
| | | | | | | APTAAPKAP | | | |
| | | | | | | PISKAPPAKAPSVPPPLSSASPPSKAPAKSP | | | |
| | | | | | | PKTPPIPKAP | | | |
| | | | | | | VAAPVSPPPKAGPSKKPPVLAPSKAPSASHG | | | |
| | | | | | | AAPVSSPP | | | |
| | | | | | | APAPPKHKKPTEAPAPAPPKHKKPTEAPAPA | | | |
| | | | | | | PPKHKKPT | | | |
| | | | | | | EAPSPAPSHHKKPAEAPAPAPASSKHKPHVE | | | |
| | | | | | | TPAPAPAK | | | |
| | | | | | | HKRPVEAPTPASEEAPTPAPAPAKHKKKKKK | | | |
| | | | | | | PKHPKKNQ | | | |
| | | | | | | KHHHHHAPAPAPVVSPPAPPLSPEPSTSEDL | | | |
| | | | | | | SVPAPAPD | | | |
| | | | | | | SPNGGMKSFVAGIWRTLVVASILVIFMA | | | |
| | | | | | | (SEQ ID NO: 976) | | | |
| | Other | | | | | | | | |
| Hordeum | AG-Peptide | AGh | QAA[E/V]OSOAAEV | | | | | | Van den |
| vulgare | | | (SEQ ID NO: 977) | | | | | | Bulck et |
| | | | OTGD (SEQ | | | | | | al. 2005 |
| | | | ID NO: 978) | | | | | | |
| Secale | AG-Peptide | AGh | YADVOSOAAEGOAVD | | | | | | Van den |
| cereale | | | (SEQ ID NO: 979) | | | | | | Bulck et |
| | | | | | | | | | al. 2005 |
| Triticum | AG-peptide, | AGh | VOSOAAQAOTA | | | | | | Van den |
| aestivum | GSP-1 | | (SEQ ID NO: 980) | | | | | | Bulck et |
| | | | YAEVOSO[A/H/M]Q | | | | | | al. 2002 |
| | | | AOTAD (SEQ ID | | | | | | |
| | | | NO: 981) | | | | | | |
| Triticum | AG-Peptide | AGh | YAEVOSOA[H/A/M] | | | | | | Van den |
| durum | | | QAOTAD (SEQ ID | | | | | | Bulck et |
| | | | NO: 982) | | | | | | al. 2005 |
| Triticum | AG-Peptide | AGh | YAEVOSOA[A/H/M] | | | | | | Van den |
| spelta | | | QAOTAD (SEQ ID | | | | | | Bulck et |
| | | | NO: 983) | | | | | | al. 2005 |
| Tritico- | AG-Peptide | AGh | YAEVOSOA[A/H/M] | | | | | | Van den |
| secale | | | QAOT[V/A]D | | | | | | Bulck et |
| | | | (SEQ ID NO: 984) | | | | | | al. 2005 |
| | | | VYAEVOSOA[A/H/M] | | | | | | |
| | | | QAOT[V/A]D | | | | | | |
| | | | (SEQ ID NO: 985) | | | | | | |
| Zea mays | Hybrid | Ara, Galh | AOOAHFPSOO (SEQ | 27 | Chromista, | >YQIJ_Locus_4908 | Core | Phacelia | Kieliszews |
| | AGP | | ID NO: 986) | | Conifers, | (Class 2, CL-EXT) | Eudicots | campan- | ki et al. |
| | HHRGP | | NGPKHHOOOSXANXO | | Asterids, | MGSHMASLVIAILVAIVSLSLPSTTSANYKY | Lepto- | ularia | 1992 |
| | | | OOEOOY | | Liverworts, | TSPPPPL | sporan- | Davallia | |
| | | | (SEQ ID NO: 987) | | Core Eudicots | KKYPPPHYIYKSPPPPPPVHKYPPPHHPIHK | giate | fejeensis | |
| | | | SOOAHH (SEQ ID | | Asterids, | SPPAHHPVYKSPPPPPHHKKPYKYKSPPPPP | Monilo- | | |
| | | | NO: 988) | 224 | Mosses, | PPVHKYPPTPHHPI | phytes | | |
| | | | NOOOOAHY (SEQ | | Rosids, Green | YKSPPPPTPHKKPYKYKSPPPPPPVYKSPPP | | | |
| | | | ID NO: 989) | | Algae, | PPPKKPY | | | |
| | | | HAOOOOFASY (SEQ | | Core Eudicots, | KYKSPPPPVYKSPPRPPPVTSHHHLHHHTKS | | | |
| | | | ID NO: 990) | | Liverworts, | PTSTSLL | | | |
| | | | AOOOOAHHHOOO | | Conifers, | HPLLQSTSLLHHHHQRNPTSTNLHHHQCTSL | | | |
| | | | (SEQ ID NO: 991) | | Basal Eudicots, | LHLHLQYTSHHHLHHHTKS | | | |
| | | | NAOOOAAHY (SEQ | | Monocots, | (SEQ ID NO: 1008) | | | |
| | | | ID NO: 992) | | Hornworts, | >OQWW_Locus_38562 | | | |
| | | | AOOOYFPTO (SEQ | | Cycadales, Red | (Class 4, non-GPI-AGP) | | | |
| | | | ID NO: 993) | | Algae, | MAALQTLAGLSLLLLVIASSLLPCSQAYWPP | | | |
| | | | AOOAPAP (SEQ ID | 4 | Lycophytes, | TPPPYSSQPPSPSYHKPPPAPLPSTPPPVYT | | | |
| | | | NO: 994) | | Leptosporangiate | PSPPIYTLPPSPPVQPPPVVWAPPPHFYKPP | | | |
| | | | SOOOHSOSOGHY | | Monilophytes, | APYAKPPVPCPPPS | | | |
| | | | (SEQ ID NO: 995) | | Commelinids, | (SEQ ID NO: 1009) | | | |
| | | | ASOOYFPOHGAY | | Euglenozoa | | | | |
| | | | (SEQ ID NO: 996) | | Leptosporangiate | | | | |
| | | | AOOOHFPSOOAANAN | | Monilophytes, | | | | |
| | | | AAOOOAHY | | Green | | | | |
| | | | (SEQ ID NO: 997) | | Algae, Red Algae | | | | |
| | | | AOOOHFPSOOOHHHO | | | | | | |
| | | | OOYA (SEQ | | | | | | |
| | | | ID NO: 998) | | | | | | |
| | | | AAOOOHFPSOO | | | | | | |
| | | | (SEQ ID NO: 999) | | | | | | |
| | | | NAAOOXAAYY | | | | | | |
| | | | (SEQ ID | | | | | | |
| | | | NO: 1000) | | | | | | |
| | | | HVOSOSENGPK | | | | | | |
| | | | (SEQ ID | | | | | | |
| | | | NO: 1001) | | | | | | |
| | | | HHOOSXAEXOOOEO | | | | | | |
| | | | OY (SEQ ID | | | | | | |
| | | | NO: 1002) | | | | | | |
| | | | SOOGGTXAPNXOI | | | | | | |
| | | | (SEQ ID NO: | | | | | | |
| | | | 1003) | | | | | | |
| | | | AOOOHF (SEQ ID | | | | | | |
| | | | NO: 1004) | | | | | | |
| | | | AOOAHFPSOO[T/A] | | | | | | |
| | | | F (SEQ ID NO: | | | | | | |
| | | | 1005) | | | | | | |
| | | | HHOOOSOAEXOOOEO | | | | | | |
| | | | OEXAOOO | | | | | | |
| | | | EFP (SEQ ID | | | | | | |
| | | | NO: 1006) | | | | | | |
| | | | HAOOOOAAHAOOOXF | | | | | | |
| | | | PSOOOAX | | | | | | |
| | | | XF (SEQ ID | | | | | | |
| | | | NO: 1007) | | | | | | |
| Zea mays | Hybrid | nd | TOTOVSHTOSOOOOY | 2 | Commelinids | >YPIC_Locus_4010 | Monocots/ | Micro- | Kieliszews |
| | | | (SEQ ID | | | (Class 4, non-GPI-AGP) | Comme- | stegium | ki et al. |
| | HRGP, | | NO: 1010) | | | MMMGGKKAAALLLALVALSLAVEIQADDTT | linids | vimineum | 1990 |
| | THRGP | | TOSOYPTOOTY (SEQ | | | GYGYGGGGYTPTPEKPPNKGPKPEKPPKEHG | | | |
| | | | ID NO: 1011) | | | HKHPKEHKPPTYTPTPKPTPPTYTPKPPTYP | | | |
| | | | TOSOKPOTOKPP | | | PPTYTPTPKPPATKPPTYPTPKPTPYTPTPK | | | |
| | | | (SEQ ID NO: | | | PPTYETPKPTPPRTTRSRSRRLPSLRHTRHP | | | |
| | | | 1012) | | | SRP (SEQ ID NO: 1017) | | | |
| | | | TOSOKPOTPKPTOO | | | | | | |
| | | | TY (SEQ ID | | | | | | |
| | | | NO: 1013) | | | | | | |
| | | | TOTOKPOATKPPTY | | | | | | |
| | | | (SEQ ID NO: | | | | | | |
| | | | 1014) | | | | | | |
| | | | TOSOKPOTHPTP | | | | | | |
| | | | (SEQ ID NO: | | | | | | |
| | | | 1015) | | | | | | |
| | | | PTOOTYTOSOKPOTP | | | | | | |
| | | | K (SEQ ID | | | | | | |
| | | | NO: 1016) | | | | | | |
| Eudicots | AGP | | | | | | | | |
| Arabidopsis | Classical | AG | AOAOAOTTVTPOOTA | | | | | | Schultz |
| thaliana | AGP, | (Yariv) | (SEQ ID | | | | | | et al. |
| | AtAGP2 | | NO: 1018) | | | | | | 2000 |
| Arabidopsis | Classical | AG | AOAOTOTATOOOATO | | | | | | Schultz |
| thaliana | AGP. | (Yariv) | OOV (SEQ | | | | | | et al. |
| | AtAGP4 | | ID NO: 1019) | | | | | | 2000 |
| Arabidopsis | Classical | AG | EILTKSSOAOSODLA | | | | | | Tan et al. |
| thaliana | AGP. | (Yariv) | DSPLI (SEQ | | | | | | 2013 |
| | AtAGP57 | | ID NO: 1020) | | | | | | |
| Daucus | DcAGP | | DEAOAOAOSOM | 1008 | Asterids, | >AUIP_Locus_6715 | Core | Phelline | Jermyn and |
| | | | (SEQ ID NO: | | Monocots, | (Class 1, GPI-AGP) | eudicot | lucida | Guthrie |
| carota | | | 1021) | | Rosids, Green | MARHLVVLALIFVALVGFVSAEGPSSSPAAT | | | 1985; |
| | | | OAOAOAO (SEQ ID | | Algae, | PSAS | | | Showalter |
| | | | NO: 1022) | | Leptosporangiate | PATTPSAAPPKDAATPTAATPAASTPSSSAP | | | 2001; |
| | | | | | Monilophytes, | TAATPDASAAPSTTTDSPASSPDTEEPSSPP | | | |
| | | | | | Basalmost | APASESTDSPLASPPAPTASSPTGDEAPAPA | | | |
| | | | | | angiosperms, | PKSSGAATLKVSAVA | | | |
| | | | | | Basal | GAAVVAGVFLF (SEQ ID NO: 1023) | | | |
| | | | | | Eudicots, | | | | |
| | | | | | Commelinids, | | | | |
| | | | | | Core | | | | |
| | | | | | Eudicots, | | | | |
| | | | | | Liverworts, | | | | |
| | | | | | Eusporangiate | | | | |
| | | | | | Monilophytes, | | | | |
| | | | | | Conifers, | | | | |
| | | | | | Mosses, | | | | |
| | | | | | Lycophytes, Red | | | | |
| | | | | | Algae, | | | | |
| | | | | | Magnoliids, | | | | |
| | | | | | Gnetales, | | | | |
| | | | | | Hornworts, | | | | |
| | | | | | Chromista, | | | | |
| | | | | | Cycadales, | | | | |
| | | | | | Euglenozoa, | | | | |
| | | | | | Chloranthales | | | | |
| Lyco- | Classical | AG | OAAAOTKPK (SEQ | 222 | Asterids, | >DLAI_Locus_3123 | Core | Solanum | Gao et al. |
| | | | ID NO: 1024) | | Rosids | (Class 1, GPI-AGP) | eudicot | lasio- | 1999; Zhao |
| persicum | AGP, | (Yariv) | AOASSOOVQSOOAOA | | Asterids | MDRKFVFLVSILCIVVASVTGQTPAAAPVGA | | phyllum | et al. |
| esculentum | LeAGP1 | | OEVATOOAV (SEQ | | | KAGTTPPAAAPTKPKTPAPATAPASAPPTAV | | | 2002 |
| | | | ID NO: 1025) | | | PVAPVTAPVT | | | |
| | | | TGQTOAAAXVGAKAG | | | APTTPVVAAPVSAPASSPPVKAPASSPPVQS | | | |
| | | | TTOOAAO (SEQ | | | PPAPAPEVATPPAVSTPPAAAPVAAPVASET | | | |
| | | | ID NO: 1026) | | | TPAPAPSKGKVKGKKGKKHNASPAPSPDMMS | | | |
| | | | AOASSOOVQSOOAOA | | | PPAPPSEAPGPSMDSDSAPSPSLNDESGAEK | | | |
| | | | OEVATOO | | | LKMLGSLVAGWAVMSWLLF | | | |
| | | | VST (SEQ | | | (SEQ ID NO: 1029) | | | |
| | | | ID NO: 1027) | | | | | | |
| | | | OOAAAXVAA (SEQ | | | | | | |
| | | | ID NO: 1028) | | | | | | |
| Lyco- | AGP | | SAYISOAOVASOO | | | | | | Zhao et |
| persicum | | | (SEQ ID NO: | | | | | | al. 2002 |
| esculentum | | | 1030) | | | | | | |
| Nicotiana | Classical | AG | LASOOAOOTADTOA | 21 | Asterids | >MKZR Locus_8106 | Core | Nico- | Du et al. |
| alata | AGP, | (Yariv) | (SEQ ID NO: | | Asterids | (Class 1, GPI-AGP) | eudicot | tiana | 1994 |
| | NaAGP1 | | 1031) | | | MAYSRMMFAFIFALVAGSAFAQAPGASPAAT | | sylves | |
| | | | FAOSGGVALPOS | | | PKASPVAPVASPPTAVVTPVSAPSQSPTTAA | | tris | |
| | | | (SEQ ID NO: | | | SPSESPLASPPAPPTADTPAFAPSGGVALPP | | | |
| | | | 1032) | | | SIGAAPAGSPTSSPNAASLNRVAVAGSAVVA | | | |
| | | | VSAOSQSOSTAA | | | IFAASLMF (SEQ ID NO: 1035) | | | |
| | | | (SEQ ID NO: | | | | | | |
| | | | 1033) | | | | | | |
| | | | IGSAOAGSOTSSPN | | | | | | |
| | | | (SEQ ID NO: | | | | | | |
| | | | 1034) | | | | | | |
| Pyrus | Classical | AGh | (Q)AOXAA* (N- | 623 | Rosids, | >QIEH_Locus_8866 | Core | Coton- | Chen et |
| communis | AGP. | (Yariv) | terminal) (SEQ | | Monocots, | (Class 1, GPI-AGP) | eudicot | easter | al. |
| | PcAGP1 | | ID NO: 1036) | | Asterids, Green | MKMGFAGFQVLMVLSLLATSCIAQAPGAAPT | | trans- | 1994 |
| | | | AKSOTATOOTATOOS | | Algae, | AS | | caucas- | |
| | | | AV (SEQ ID | | Monocots/ | PPTATPPTATPPTATPPSAVPVPSPSKTPTV | | icus | |
| | | | NO: 1037) | | Commelinids, | SPTPSPVTAPTPSASPPASTPASTPSAESPS | | | |
| | | | VTAOTOSASOOSSTO | | Basal Eudicots, | SPPAPSGPSPNSPPADALPPSGTSAISRVVI | | | |
| | | | ASTXA (SEQ | | Magnoliids, | AGTALAGVFFAVVLA | | | |
| | | | ID NO: 1038) | | Basalmost | (SEQ ID NO: 1039) | | | |
| | | | | | angiosperms, | | | | |
| | | | | | Lycophytes, Core | | | | |
| | | | | | Eudicots, | | | | |
| | | | | | Liverworts, | | | | |
| | | | | | Conifers, | | | | |
| | | | | | Leptosporangiate | | | | |
| | | | | | Monilophytes, | | | | |
| | | | | | Chromista | | | | |
| | | | | | (Algae), | | | | |
| | | | | | Mosses, | | | | |
| | | | | | Glaucophyta | | | | |
| | | | | | (Algae) | | | | |
| Pyrus | Copurified | AGh | LSOKKSOTAOSOSST | 5 | Rosids | >EAVM_Locus_5196 | Core | Amelan- | Chen et |
| communis | with | (Yariv) | OOT (SEQ ID | | | (Class 1, GPI-AGP) | eudicot | chier | al. 1994 |
| | PcAGP1 | | NO: 1040) | | | MAQFSGVVMVLMASLLLASTGAQSPASSPAK | | can- | |
| | | | LSOKKSOTAOSOSST | | | SPALSPKKSPTAPSPSSTPPTAATPSPSSTS | | adensis | |
| | | | OOTT (SEQ ID | | | PSTSPSTSPVADSPPSPLSSSPSPSPSSVSR | | | |
| | | | NO: 1041) | | | SPSEAPAPANGAVLNR | | | |
| | | | [V/S][P/S]XOVQS | | | LSVSASVAAGIFAAVLVM | | | |
| | | | OASOOOTT (SEQ | | | (SEQ ID NO: 1044) | | | |
| | | | ID NO: 1042) | | | | | | |
| | | | XXOOAAOVXA[O/S] | | | | | | |
| | | | (SEQ ID NO: | | | | | | |
| | | | 1043) | | | | | | |
| Rosa Pauls | AGP | AGh | DAOAOSOV (SEQ | 1 | Rosids | >DZTK_Locus_20515 | Core | Batis | Komalavil |
| Scarlet | | (Yariv) | ID NO: 1045) | | | (Class 4, non-GPI-AGP) | eudicot | maritima | as et al. |
| | | | | | | MARFDFLLVATVLLLINFALAQDSPVSAPSP | | | 1991 |
| | | | | | | VQVPDFEPADSSPALVPASQPETAFSPPSPP | | | |
| | | | | | | PAEAPEISSFSPAKSPEISSPPAPAPVPTAP | | | |
| | | | | | | APPPSEPPVSPSEEPDAPAPSPVTDGEPSEI | | | |
| | | | | | | SDLSADEGSKTEKGMSGGKKFGVAFGVLAGA | | | |
| | | | | | | GLVGLGVFVYRKRQENIKRARYSYA | | | |
| | | | | | | AREFL (SEQ ID NO: 1046) | | | |
| | Extensin | | | | | | | | |
| Arabidopsis | AtExt3 | AG: | NYFYSSOOOOVKHYT | 374 | Asterids, | >IPWB_Locus_2214 | Core | Brassica | Cannon et |
| thaliana | | Ara:NG | OOVK [N- | | Mosses, | (Class 2, CL-EXT) | eudicot | nigra | al. 2008 |
| | | | term] (SEQ ID | 26 | Rosids, Core | MGSPMASLAATLLILALSLGFVSETTANYYY | | | |
| | | | NO: 1047) | | Eudicots, Green | SSPPPPVKHYTPPVYKSPPPPKKDYEYKSPP | | | |
| | | | SOOOOVK (SEQ ID | | Algae, | PPVKHYSPPPVYKSPPPPKKHYEYKSPPPPV | | | |
| | | | NO: 1048) | | Liverworts, | YKSPPPPVYHSPPPPKKHYEYKS | | | |
| | | | HYSOOOVY (SEQ | | Basal Eudicots, | (SEQ ID NO: 1050) | | | |
| | | | ID NO: 1049) | | Leptosporangiate | | | | |
| | | | | | Monilophytes, | | | | |
| | | | | | Commelinids, | | | | |
| | | | | | Monocots, | | | | |
| | | | | | Basalmost | | | | |
| | | | | | angiosperms, | | | | |
| | | | | | Cycadales, | | | | |
| | | | | | Hornworts, | | | | |
| | | | | | Lycophytes, | | | | |
| | | | | | Chromista, | | | | |
| | | | | | Magnoliids, | | | | |
| | | | | | Conifers | | | | |
| | | | | | Rosids, Core | | | | |
| | | | | | Eudicots, | | | | |
| | | | | | Chromista, | | | | |
| | | | | | Green Algae | | | | |
| Beta | Classical | Ara | SOOVHK (SEQ | 78 | Conifers, Core | >FVXD_Locus_2537 | Core | Beta | Li et al. |
| vulgaris | extensin, | | ID NO: 1051) | | Eudicots, | (Class 20, shared bias EXT | eudicot | maritima | 1990 |
| | P1-type | | YPOOTOVYK (SEQ | | Rosids, | (SPn&Y)) | | | |
| | | | ID NO: 1052) | 64 | Asterids, Basal | MGKLGGMASLVATLLVAFVSLSLPAQTIADY | | | |
| | | | SOOOTOSP (SEQ | | Eudicots, Green | TYSSPPPPVHHEMPPKGHYSPLPPTPVYKSP | | | |
| | | | ID NO: 1053) | 16 | Algae | PVHTYPPPSPIYKSPPVHEYPPPTPVYKSPP | | | |
| | | | SOOVHEYPOOT | 24 | Core Eudicots, | VHKYPPPTPVY | | | |
| | | | [X/O]VYK (SEQ | | Asterids | KSPP (SEQ ID NO: 1055) | | | |
| | | | ID NO: 1054) | | Green Algae, | | | | |
| | | | | | Commelinids, | | | | |
| | | | | | Conifers, | | | | |
| | | | | | Asterids, | | | | |
| | | | | | Rosids, | | | | |
| | | | | | Lycophytes | | | | |
| | | | | | Core Eudicots | | | | |
| Lyco- | Classical | Ara | SOOOO[X]TOVYK | 368 | Basal Eudicots, | >ZSNV_Locus_2413 | Basal | Papaver | Smith et |
| persicum | extensin, | | (SEQ ID NO: | | Asterids, Core | (Class 2, CL-EXT) | eudicot | bract- | al. 1984; |
| esculentum | P1-type | | 1056) | | Eudicots, | MANRGFSMASSITLLVVLISLSWPFFAEADG | | eatum | Smith et |
| | | | SOOOOTOVYK (SEQ | | Magnoliids, | YYSYKSPPPPTPVYKYASPPPPVYIYKSPPP | | | al. |
| | | | ID NO: 1057) | | Rosids, | PTPVYKSPPPPTPVYKYKSPPPPTPVYKYKS | | | 1986 |
| | | | SOOOOVKPYHPTOV | | Monocots, | PPPPTPVYK (SEQ ID NO: 1059) | | | |
| | | | YK (SEQ ID | | Conifers, | | | | |
| | | | NO: 1058) | | Commelinids, | | | | |
| | | | | | Basalmost | | | | |
| | | | | | angiosperms | | | | |
| Lyco- | Classical | Ara | SOOOOVYK (SEQ | 514 | Conifers, | >LQJY_Locus_2340 | Core | Solanum | Smith et |
| persicum | extensin, | | ID NO: 1060) | | Leptosporangiate | (Class 2, CL-EXT) | eudicot | xantho- | al. 1984; |
| esculentum | P2-type | | | | Monilophytes, | MAKIAYLLITLLVALVSLSFPSECKENYYYT | | carpum | Smith et |
| | | | | | Lycophytes, | SPPPPIPIYKYKSPPPPVYKYKSPPPPDYKY | | | al. |
| | | | | | Rosids, | KSPPPPVYKYKSPPPPPPVYKYKSPPPPVYK | | | 1986 |
| | | | | | Monocots, | YKSPPPPPPVYKYKSPPPPVYKYKSPPPPPP | | | |
| | | | | | Asterids, | VYKYKSPPPPVYKYKSP | | | |
| | | | | | Hornworts, | PPPPPVYKYKSPPP (SEQ ID NO: 1061) | | | |
| | | | | | Eusporangiate | | | | |
| | | | | | Monilophytes, | | | | |
| | | | | | Magnoliids, | | | | |
| | | | | | Basal Eudicots, | | | | |
| | | | | | Chloranthales, | | | | |
| | | | | | Core | | | | |
| | | | | | Eudicots, | | | | |
| | | | | | Basalmost | | | | |
| | | | | | angiosperms, | | | | |
| | | | | | Liverworts, | | | | |
| | | | | | Green Algae, | | | | |
| | | | | | Commelinids, | | | | |
| | | | | | Mosses, | | | | |
| | | | | | Cycadales | | | | |
| Lyco- | Classical | | SOOOOSOSOOOOYY | 412 | Monocots, Core | >CPOC_Locus_8084 | Core | Convo- | Lamport |
| persicum | extensin, | | YK (SEQ ID | | Eudicots/ | (Class 2, CL-EXT) | eudicot | lvulus | 1977 |
| esculentum | P3-type | | NO: 1062) | | Asterids, | MAAHGGDPGRGRLLPQILVALVVLAAASVVS | | arvensis | |
| | | | | | Conifers, Core | GDPYVYASPPPPTYEYKSPPPPSPSPPPPYE | | | |
| | | | | | Eudicots/Rosids, | YKSPPPPSPSPPPPYEYKSPPPPSPSPPPPY | | | |
| | | | | | Core Eudicots, | YYKSPPPPSPSPPPPYYYKSPPPPSPSPPPP | | | |
| | | | | | Basalmost | YYYKSPPPPSPSPPPPYYYHSPPPPKKSPPP | | | |
| | | | | | angiosperms, | PYYYTSPPPPVKSPPPLYYYQSPPPPKKSPP | | | |
| | | | | | Magnoliids, | PPYYYHSPPPPVKSPPPPYYYNSPPPPKKSP | | | |
| | | | | | Basal | PPPYYYHSPPPPYYYNSPPP (SEQ ID | | | |
| | | | | | Eudicots, | NO: 1063) | | | |
| | | | | | Lycophytes, | | | | |
| | | | | | Liverworts | | | | |
| Lyco- | Classical | Ara | NYQYSOOOOOOK | | | | | | Brown- |
| persicum | extensin | (TLC)i | (SEQ ID NO: | | | | | | leader |
| esculentum | | | 1064) | | | | | | and Dey |
| | | | | | | | | | 1993 |
| Medicago | Classical | Ara | [K/A]SOOOOAOVY | 48 | Lycophytes, | >NIGS Locus_899 | Core | Helio- | Frueauf |
| | | | | | Core Eudicots/ | (Class 2, CL-EXT) | eudicot | tropium | et |
| truncutula | extensin, | | (SEQ ID NO: | 368 | Asterids, | MASPLFAILVALVSLNLLPSQTTANYPYASP | | kar- | al. 2000 |
| | P1-type | | 1065) | | Conifers, Basal | HPPPYHYKSMPPIPKYPPHHHPIYKSPRPLP | | winskyi | |
| | | | K[A/S]OOOOTOVY | | Eudicots, Core | PIHKPPPHHHPVYKSPPPPPSVHKYPPPSPS | | | |
| | | | (SEQ ID NO: | | Eudicots, Core | HKKPYKKSPPPPILVYHSPPPPQVYKSPPLP | | | |
| | | | 1066) | | Eudicots/Rosids, | PPVYKSPPPKKPYKSPPPPTSVYHSPPPPPT | | | |
| | | | | | Magnoliids | KKPYKSPPPPPTPVYHSPPPPPTKKPYKSPP | | | |
| | | | | | Basal Eudicots, | PPTPVYHSPPPPPTKKPYKYKSPPPPTPVYK | | | |
| | | | | | Core | SPPPVYRSPPPPHKKPYKYKSPPPPAPVYHS | | | |
| | | | | | Eudicots, | PPPTPHHKKPYKYKSPPPPPPIYKSPPPPSH | | | |
| | | | | | Asterids, | HYYYSSPPHHY (SEQ ID NO: 1067) | | | |
| | | | | | Magnoliids, | | | | |
| | | | | | Rosids, | | | | |
| | | | | | Monocots, | | | | |
| | | | | | Conifers, | | | | |
| | | | | | Commelinids, | | | | |
| | | | | | Basalmost | | | | |
| | | | | | angiosperms | | | | |
| Medicago | Extensin- | Ara | KSOOOSS (SEQ | 73 | Core Eudicots, | >LQJY_Locus_34577 | Core | Solanum | Frueauf |
| truncutula | like | | ID NO: 1068) | | Commelinids, | (Class 19, shared bias AGP) | eudicot | xantho- | et al. |
| | | | | | Mosses, | MGFTSVKAIVLIQVLALVLDSSSKLSFGEVT | | carpum | 2000 |
| | | | | | Monocots, | EDWSLDNYQDNEVISNFKSSPGRSTPSTPKS | | | |
| | | | | | Conifers, Basal | SPSPSLPLSSTPPPTKSSPSSLLLAKSPPPS | | | |
| | | | | | Eudicots | SSPSPPPTKSPPSPSSSPPIYASSPLAKSPA | | | |
| | | | | | Rosids, | PSPLSPSSSPSPPTKSPPPSLAKSPPPIAKS | | | |
| | | | | | Asterids, | PPPTLAKSPSPSVKSPPSTSSSPLLPTKSPP | | | |
| | | | | | Lycophytes, | PSLVKSSPPTTNYPLPPPVKSS (SEQ ID | | | |
| | | | | | Green Algae, | NO: 1069) | | | |
| | | | | | Leptosporangiate | | | | |
| | | | | | Monilophytes, | | | | |
| | | | | | Magnoliids, | | | | |
| | | | | | Eusporangiate | | | | |
| | | | | | Monilophytes | | | | |
| Medicago | P3-type | Ara | K[A/S]OOOX[A/S] | 1016 | Monocots, | see >CPOC_Locus_8084 | | | Frueauf |
| truncutula | extensin | | OSOOOXY (SEQ | 27 | Leptosporangiate | | | | et al. |
| | | | ID NO: 1070) | | Monilophytes, | | | | 2000 |
| | | | KSOOOOSOSOOOO | | Asterids, | | | | |
| | | | [X/O]Y (SEQ | | Conifers, | | | | |
| | | | ID NO: 1071) | | Rosids, Basal | | | | |
| | | | | | Eudicots, Core | | | | |
| | | | | | Eudicots, | | | | |
| | | | | | Basalmost | | | | |
| | | | | | angiosperms, | | | | |
| | | | | | Magnoliids, | | | | |
| | | | | | Lycophytes, | | | | |
| | | | | | Eusporangiate | | | | |
| | | | | | Monilophytes, | | | | |
| | | | | | Commelinids, | | | | |
| | | | | | Green Algae, | | | | |
| | | | | | Cycadales, | | | | |
| | | | | | Chromista, | | | | |
| | | | | | Liverworts | | | | |
| | | | | | Conifers, | | | | |
| | | | | | Basalmost | | | | |
| | | | | | angiosperms, | | | | |
| | | | | | Rosids, | | | | |
| | | | | | Lycophytes, | | | | |
| | | | | | Monocots, | | | | |
| | | | | | Asterids, | | | | |
| | | | | | Basal Eudicots | | | | |
| Medicago | Extensin | Ara | [H/D]SOOOOVH | 126 | Rosids, Basal | >SXCE Locus_19111 (Class 20, | Core | Physo- | Frueauf |
| truncutula | | | (SEQ ID | | Eudicots, | shared bias EXT (SPn&Y)) | eudicot | carpus | et al. |
| | | | NO: 1072) | | Leptosporangiate | MGNSTALAFMEEKLFPCSLRLKVGLILLAIF | | opuli- | 2000 |
| | | | | | Monilophytes, | AGLTSEEETGLLCISDCTTCPIICSPPPPQP | | folius | |
| | | | | | Monocots, Core | ESSNPPPSPDSPPPPVHHSPAPQYYYTSPPP | | | |
| | | | | | Eudicots, | SPPPPSPPKKSPSPPSSSSTYSSKGSPPAPW | | | |
| | | | | | Asterids, | NYFYNLPPSGPDQVPPTTGVLQHNNSFPYYY | | | |
| | | | | | Green Algae, | FYASEASSLSVH (SEQ ID NO: 1073) | | | |
| | | | | | Magnoliids, | | | | |
| | | | | | Lycophytes, | | | | |
| | | | | | Mosses, | | | | |
| | | | | | Conifers | | | | |
| | PRP | | | | | | | | |
| Glycine max | SbPRP1 | nd | XYEKPOIYKPOVYT | 9 | Rosids, | >ZUQW Locus 37 | Core | Glycine | Lindstrom |
| | | | (SEQ ID NO: | | Asterids, | (Class 18, PRP bias Tyr) | eudicot | soja | and |
| | 35 kDa | | 1074) | | Leptosporangiate | MRNMASLSSSLVLLLAALILSPQVLADYEKP | | | Vodkin |
| | | | | | Monilophytes | PIYKPPVYTPPVYKPPIYKPPVYTPPVYKPP | | | 1991 |
| | | | | | | VEKPPVYKPPVYKPPVEKPPVYKPPVYKPPI | | | |
| | | | | | | YKPPVV (SEQ ID NO: 1075) | | | |
| Glycine max | SbPRP1 | ″not | NYENPOVYKPOTEKP | 6 | Rosids | >PFSA Locus_1393 | Core | Glycine | Kleis-San |
| | | highly | OVY (SEQ | | | (Class 18, PRP bias Tyr) | eudicot | soja | Francisco |
| | | glyco- | ID NO: 1076) | | | MASLSSLVLLLAALILSPQVLANYENPPVYK | | | and |
| | | sylated″ | | | | PPTEKPPVYKPPVEKPPVYKPPVENPPIYKP | | | Tierney |
| | | (Datta | | | | PVEKPPVYKPPVDTASYKPPIYKPPVYKPPV | | | 1990 |
| | | 1989) | | | | EKPPVYKPPV (SEQ ID NO: 1077) | | | |
| Glycine max | RPRP1 | ″not | POVYKPOVYK (SEQ | 59 | Leptosporangiate | see ZUQW_Locus_37 | | | Averyhart- |
| | | glyco- | ID NO: 1078) | | Monilophytes, | | | | Fullard et |
| | | sylated″ | | | Lycophytes, | | | | al. 1988; |
| | | | | | Rosids, | | | | Datta et |
| | | | | | Cycadales, | | | | al. 1989 |
| | | | | | Conifers, | | | | |
| | | | | | Basal Eudicots, | | | | |
| | | | | | Asterids | | | | |
| Glycine max | RPRP2 | ″not | POVYKPOVEK (SEQ | 95 | Leptosporangiate | see PFSA Locus 1393 | | | Averyhart- |
| | | glyco- | ID NO: 1079) | | Monilophytes, | | | | Fullard et |
| | | sylated″ | | | Rosids, Asterids | | | | al. 1988; |
| | | | | | | | | | Datta et |
| | | | | | | | | | al. 1989 |
| Medicago | PRP4-type | Ara | KAOVGKPOVY (SEQ | | | | | | Frueauf |
| truncutula | | | ID NO: 1080) | | | | | | et al. |
| | | | | | | | | | 2000 |
| | Other | | | | | | | | |
| Arabidopsis | AG-peptide | AG | [TVS][EDL]AOAO | | | | | | Schultz |
| thaliana | | (Yariv) | [SA]OTS-gpi | | | | | | et |
| | | | anchor (SEQ ID | | | | | | al. 2004 |
| | | | NO: 1081) | | | | | | |
| Arabidopsis | AG-peptide | AG | [TVS][EDL]AOAO | | | | | | Schultz |
| thaliana | | (Yariv) | [SA]OTSGS-gpi | | | | | | et |
| | | | anchor (SEQ ID | | | | | | al. 2004 |
| | | | NO: 1082) | | | | | | |
| Arabidopsis | AG-peptide, | AG | HEGHHHHAOAOAOGO | | | | | | Schultz |
| thaliana | AtAGP24 | (Yariv) | AS-gpi | | | | | | et |
| | | | anchor (SEQ ID | | | | | | al. 2004 |
| | | | NO: 1083) | | | | | | |
| Arabidopsis | Hybrid | AG:Arah | AOVSSOOAK[PO/OP] | | | | | | Hijazi et |
| thaliana | HRGP, | (Yariv) | VK[PO/OP]VY | | | | | | al. 2012 |
| | AtAGP31 | | [PO/OP]TK (SEQ | | | | | | |
| | (GaRSGP | | ID NO: 1084) | | | | | | |
| | orthologue) | | AOVK[PO/OP]TK | | | | | | |
| | | | [PO/OP]VK[PO/OP] | | | | | | |
| | | | VY[PO/OP]TK | | | | | | |
| | | | (SEQ ID NO: | | | | | | |
| | | | 1085) | | | | | | |
| | | | AOVK[PO/OP]VSOO | | | | | | |
| | | | TK[PO/OP]VT[PC/ | | | | | | |
| | | | OP]VYPPK (SEQ | | | | | | |
| | | | ID NO: 1086) | | | | | | |
| | | | AOVK[PO/OP]TK | | | | | | |
| | | | [PO/OP]VK[PO/OP] | | | | | | |
| | | | VSOOAK[PO/OP]VS | | | | | | |
| | | | OOAK[PO/OP] | | | | | | |
| | | | VK[PO/OP]VY | | | | | | |
| | | | [PO/OP]TK (SEQ | | | | | | |
| | | | ID NO: 1087) | | | | | | |
| Nicotiana | Hybrid | Gal: Ara | KHHDHLSOAQA | 2 | Asterids | >DLJZ Locus_8134 | Core | Solanum | Sommer- |
| | | | (SEQ ID NO: | | | (Class 19, shared bias AGP) | eudicot | pty- | Knudsen |
| alata | HRGP, | | 1088) | | | MAKALVLFQLSVLLLSSFTVVSHAHDHLSPA | | canthum | et al. |
| | GaRSGP | | HLSOAQAOK (SEQ | | | QAPKPHKGGHHHHHSPAPSPISYTPTKPPTK | | | 1996 |
| | | | ID NO: 1089) | | | APTKPPTKAPAYSPSKPPAKPPVKPPTPSPS | | | |
| | | | | | | PSPSPYYPTRKPVVVRGLVY (SEQ ID | | | |
| | | | | | | NO: 1090) | | | |
| Nicotiana | Chimeric | AG (Yariv) | aKSKFMIIP[A/O] | | | | | | Mau et |
| alata | AGP, | | SXTX[A/O] (SEQ | | | | | | al. |
| | NaAGP2 | | ID NO: 1091) | | | | | | 1995 |
| | | | [n-term cDNA R | | | | | | |
| | | | not A] | | | | | | |
| Nicotiana | Hybrid | AG: | LFFGKSOKKSOSSOT | 45 | Green Algae, | >YZVJ Locus_6899 | Core | Cepha- | Schultz |
| alata | HRGP, 120 | Ara: Galg | OVNKPS( SEQ | | Asterids, | (Class 19, shared bias AGP) | eudicot | lotus | et |
| | kDa | | ID NO: 1092) | | Rosids, Red | MGSKNCTAIFFMFMIFMLISLPPIYACGTC | | folli- | al. 1997 |
| | | | [A/P]XVVEPO | | Algae, Core | TQPHPPPKHRPGKPGHPKTPHPEPPTPKYPP | | cularis | |
| | | | (SEQ ID NO: | | Eudicots, | FHGGPPKVSPPSKNPPSPPVVEPPIIISPPP | | | |
| | | | 1093) | | Basalmost | VINPPVINPPVIVPP | | | |
| | | | KPOOXAYTQP (SEQ | | angiosperms, | (SEQ ID NO: 1098) | | | |
| | | | ID NO: 1094) | | Conifers, | | | | |
| | | | SSOOXQDAYD (SEQ | | Chromista, | | | | |
| | | | ID NO: 1095) | | Monocots, Mosses | | | | |
| | | | VNPGPG (SEQ ID | | | | | | |
| | | | NO: 1096) | | | | | | |
| | | | LPOOSIHPAG (SEQ | | | | | | |
| | | | ID NO: 1097) | | | | | | |
| Parthenium | Par h1 | | KVCEKPSKTWFGNCK | | | | | | Gupta |
| hystero- | | | DTEKCDKRCMEWEGA | | | | | | 2000 |
| phoruS | | | KHGACHQRESKYMCF | | | | | | |
| | | | CYFDCDPKKNPGOOO | | | | | | |
| | | | GAOGTOGTOAOOGEG | | | | | | |
| | | | EGCAOOGXXAEGOA | | | | | | |
| | | | (SEQ ID | | | | | | |
| | | | NO: 1099) | | | | | | |
| Pyrus | Chimeric | AG (Yariv) | AEAEAOTOALQVVAE | | | | | | Mau et |
| commnis | AGP, | | AOELVOT (SEQ ID | | | | | | al. 1995 |
| | PcAGP2 | | NO: 1100) | | | | | | |
| | | | OVOTOSY (SEQ | | | | | | |
| | | | ID NO: 1101) | | | | | | |
| | | | AEAEAOTOALQVVAE | | | | | | |
| | | | AOEL (SEQ | | | | | | |
| | | | ID NO: 1102) | | | | | | |
| | | | VVAEAOELVOTOVOT | | | | | | |
| | | | OS (SEQ ID | | | | | | |
| | | | NO: 1103) | | | | | | |
| | | | LVOTOVOTOSY | | | | | | |
| | | | (SEQ ID NO: | | | | | | |
| | | | 1104) | | | | | | |
| Senegalia | Hybrid | AG: | SOOO(OTS)LSOSOT | | | | | | Goodru |
| senegal | HRGP, | Ara:NG | OTOO(OL)GP | | | | | | et al. |
| | Gum arabic | 20:70:10 | H (SEQ ID NO: | | | | | | 2000 |
| | glycoprotein | | 1105) | | | | | | |
| Solanum | Chimeric | pentoses | RNRPYITOSOOEASO | | | | | | Pearce et |
| nigrum | HRGP, | | STKQ (SEQ ID | | | | | | al. 2009 |
| | systemin | | NO: 1106) | | | | | | |
| | | | GRHDHVLPOOSOKHE | | | | | | |
| | | | PIIGQ (SEQ | | | | | | |
| | | | ID NO: 1107) | | | | | | |
| | | | GRHDHVOAOOAOKPE | | | | | | |
| | | | DEQGQ | | | | | | |
| | | | (SEQ ID | | | | | | |
| | | | NO: 1108) | | | | | | |
| Solanum | Chimeric | nd | CKLPSOOOOOXOO | 1 | Leptosporangiate | >LHDP_Locus_13011 | Core | Oenothera | Kieliszews |
| tuberosum | HRGP, | | (SEQ ID NO: | | Monilophytes | (Class 19, shared bias AGP) | eudicot | gaura | ki et al. |
| | Lectin | | 1109) | | | MDSSISFFLIIVLQLLSGLAPIIADKSYISY | | | 1994 |
| | | | LPSOOOOOOXXSOOO | | | CALRQKGPCYPPPPPSPPPPPPPSPPSPPHL | | | |
| | | | XXO (SEQ | | | SPSPSPSPPSPPYLSPSPSPPPSPPPHPSQP | | | |
| | | | ID NO: 1110) | | | PPPPPSPPSPPSPSPPPSLLPSPPPPPPPSP | | | |
| | | | LPSOOOOOO(X/H) | | | PSPPSPPPSPTALPP | | | |
| | | | OSOOOOSO (SEQ | | | TNITQGNGNTGIYLHLLKQ | | | |
| | | | ID NO: 1111) | | | (SEQ ID NO: 1113) | | | |
| | | | OSOOOOSOOXO | | | | | | |
| | | | (SEQ ID NO: | | | | | | |
| | | | 1112) | | | | | | |
| Solanum | Chimeric | ″variable | XASTOSPPPLPYPQX | | | | | | Allen et |
| | | | GLKKPGG (SEQ | | | | | | al. 1996 |
| | | | ID NO: 1114) | | | | | | |
| tuberosum | HRGP, | glyco- | DASTOSPPPLPDP | | | | | | |
| | Lectin | sylation″ | (SEQ ID NO: | | | | | | |
| | | | 1115) | | | | | | |
| | | | MPYPEGRSGYQVYSK | | | | | | |
| | | | AQLETXL (SEQ ID | | | | | | |
| | | | NO: 1116) | | | | | | |
| | | | XQPXOSPOOOSOOXO | | | | | | |
| | | | LOOLO (SEQ | | | | | | |
| | | | ID NO: 1117) | | | | | | |
| Zinnia | Chimeric | AG | AHQTGAOAOAADC | | | | | | Motose et |
| elegans | AGP, | (Yariv) | (SEQ ID NO: | | | | | | al. 2004 |
| | ZeXyl1 | | 1118) | | | | | | |

### NUMBERED EMBODIMENTS OF THE DISCLOSURE

Notwithstanding the appended claims, the disclosure sets forth the following numbered embodiments

### Embodiment Set 1: Stably Transformed Plant Expressing a Fusion Protein Comprising Bovine Kappa-Casein and Bovine Beta-Lactoglobulin

1. A stably transformed plant, comprising in its genome: a recombinant DNA construct encoding a fusion protein, the fusion protein comprising: a) bovine kappa-casein; and b) bovine beta-lactoglobulin, wherein the fusion protein is stably expressed in the plant.

1.1 The stably transformed plant of embodiment 1, wherein at least one of the bovine kappa-casein or beta-lactoglobulin lack a bovine signal peptide.

1.2 The stably transformed plant of embodiment 1, wherein at least one of the bovine kappa-casein or beta-lactoglobulin is a truncated protein, lacking a bovine secretion signal.

2. The stably transformed plant of embodiment 1, wherein the fusion protein comprises, in order from N-terminus to C-terminus, the kappa-casein and the beta-lactoglobulin.

3. The stably transformed plant of embodiment 1, wherein the fusion protein comprises a protease cleavage site.

4. The stably transformed plant of embodiment 3, wherein the protease cleavage site is a chymosin cleavage site.

5. The stably transformed plant of embodiment 1, wherein the fusion protein comprises a signal peptide.

6. The stably transformed plant of embodiment 5, wherein the signal peptide is located at the N-terminus of the fusion protein.

7. The stably transformed plant of embodiment 1, wherein the plant is soybean.

8. The stably transformed plant of embodiment 1, wherein the recombinant DNA construct comprises codon-optimized nucleic acids for expression in the plant.

9. The stably transformed plant of embodiment 1, wherein the fusion protein has a molecular weight of 30 kDa to 50 kDa.

10. The stably transformed plant of embodiment 1, wherein the fusion protein is expressed at a level at least 2-fold higher than kappa-casein expressed individually in a plant.

11. The stably transformed plant of embodiment 1, wherein the fusion protein accumulates in the plant at least 2-fold higher than kappa-casein expressed without beta-lactoglobulin.

12. The stably transformed plant of embodiment 1, wherein the fusion protein is stably expressed in the plant in an amount of 1% or higher per total protein weight of soluble protein extractable from the plant.

13. A transgenic soybean plant, comprising: a recombinant DNA construct encoding a fusion protein, the fusion protein comprising: a) bovine kappa-casein; and b) bovine beta-lactoglobulin, wherein the fusion protein is expressed in the soybean plant.

14. The transgenic soybean plant of embodiment 13, wherein the fusion protein comprises, in order from N-terminus to C-terminus, the kappa-casein and the beta-lactoglobulin.

15. The transgenic soybean plant of embodiment 13, wherein the fusion protein comprises a protease cleavage site.

16. The transgenic soybean plant of embodiment 13, wherein the fusion protein comprises a chymosin cleavage site.

17. The transgenic soybean plant of embodiment 13, wherein the fusion protein has a molecular weight of 30 kDa to 50 kDa.

18. A transgenic soybean plant, comprising: a recombinant DNA construct encoding a fusion protein, the fusion protein comprising a bovine casein and bovine beta-lactoglobulin.

19. The transgenic soybean plant of embodiment 18, wherein the fusion protein comprises, in order from N-terminus to C-terminus, the bovine casein and the beta-lactoglobulin.

20. The transgenic soybean plant of embodiment 18, wherein the fusion protein comprises a protease cleavage site.

21. The transgenic soybean plant of embodiment 18, wherein the fusion protein comprises a chymosin cleavage site.

22. The transgenic soybean plant of embodiment 18, wherein the fusion protein has a molecular weight of 30 kDa to 50 kDa.

### Embodiment Set 2: Stably Transformed Plant Expressing a Fusion Protein Comprising Kappa-Casein or Para-Kappa-Casein and Beta-Lactoglobulin

1. A recombinant fusion protein, comprising: a) full-length kappa-casein or para-kappa-casein; and b) beta-lactoglobulin.

1.1 The recombinant fusion protein of embodiment 1, wherein at least one of the kappa-casein, para kappa-casein, or beta-lactoglobulin lack a bovine secretion signal peptide.

1.2 The recombinant fusion protein of embodiment 1, wherein at least one of the kappa-casein, para kappa-casein, or beta-lactoglobulin is a truncated milk protein, lacking a bovine secretion signal peptide.

2. The recombinant fusion protein of embodiment 1, wherein the fusion protein comprises, in order from N-terminus to C-terminus, the full-length kappa-casein or the para-kappa-casein and the beta-lactoglobulin.

3. The recombinant fusion protein of embodiment 1, further comprising a protease cleavage site.

4. The recombinant fusion protein of embodiment 3, wherein the protease cleavage site is a chymosin cleavage site.

5. The recombinant fusion protein of embodiment 1, further comprising a signal peptide.

6. The recombinant fusion protein of embodiment 5, wherein the signal peptide is located at the N-terminus of the fusion protein.

7. The recombinant fusion protein of embodiment 1, wherein the fusion protein comprises the full-length kappa-casein.

8. The recombinant fusion protein of embodiment 1, wherein the fusion protein comprises para-kappa-casein.

9. The recombinant fusion protein of embodiment 1, wherein the fusion protein has a molecular weight of 30 kDa to 50 kDa.

10. A plant transformed to express the recombinant fusion protein of embodiment 1, wherein the fusion protein is expressed in the plant in an amount of 1% or higher per total protein weight of soluble protein extractable from the plant.

11. A plant transformed to express the recombinant fusion protein of embodiment 1, wherein the fusion protein is expressed in the plant at a level at least 2-fold higher than kappa-casein expressed individually in a plant.

12. A plant transformed to express the recombinant fusion protein of any one of embodiments 1-1.2, wherein the fusion protein accumulates in the plant at least 2-fold higher than kappa-casein expressed without beta-lactoglobulin.

13. A fusion protein comprising kappa-casein and beta-lactoglobulin, wherein the kappa-casein is full-length kappa-casein comprising an amino acid sequence having at least 90% sequence identity to SEQ ID NO: 4 or the kappa-casein is para-kappa-casein comprising an amino acid sequence having at least 90% sequence identity to SEQ ID NO: 2 and wherein the beta-lactoglobulin is full-length beta-lactoglobulin comprising an amino acid sequence having at least 90% sequence identity to SEQ ID NO: 10.

14. The fusion protein of embodiment 13, wherein the kappa-casein is full-length kappa-casein comprising an amino acid sequence SEQ ID NO: 4.

15. The fusion protein of embodiment 13, wherein the kappa-casein is para-kappa-casein comprising an amino acid sequence SEQ ID NO: 2.

16. The fusion protein of embodiment 13, wherein the beta-lactoglobulin comprises the amino acid sequence SEQ ID NO: 10.

17. The fusion protein of embodiment 13, further comprising a protease cleavage site between the kappa-casein and beta-lactoglobulin.

18. The fusion protein of embodiment 17, wherein the protease cleavage site is a chymosin cleavage site.

19. The fusion protein of embodiment 13, further comprising a signal peptide.

20. A nucleic acid molecule encoding a fusion protein comprising kappa-casein and beta-lactoglobulin, wherein the kappa-casein is full-length kappa-casein comprising an amino acid sequence having at least 90% sequence identity to SEQ ID NO: 4 or the kappa-casein is para-kappa-casein comprising an amino acid sequence having at least 90% sequence identity to SEQ ID NO: 2 wherein the beta-lactoglobulin is full-length beta-lactoglobulin comprising an amino acid sequence having at least 90% sequence identity to SEQ ID NO: 10.

21. The nucleic acid molecule of embodiment 20, wherein the nucleic acid sequence is codon optimized for expression in a plant.

22. The nucleic acid molecule of embodiment 21, wherein the plant is soybean.

23. An expression vector comprising the nucleic acid molecule of embodiment 20.

24. A host cell comprising the expression vector of embodiment 23.

25. The host cell of embodiment 24, wherein the host cell is selected from the group consisting of plant cells, bacterial cells, fungal cells, and mammalian cells.

26. The host cell of embodiment 25, wherein the host cell is a plant cell.

27. A plant stably transformed with the nucleic acid molecule of embodiment 20.

28. The plant of embodiment 27, wherein the plant is a monocot selected from the group consisting of turf grass, maize, rice, oat, wheat, barley, sorghum, orchid, iris, lily, onion, palm, and duckweed.

29. The plant of embodiment 27, wherein the plant is a dicot selected from the group consisting of Arabidopsis, tobacco, tomato, potato, sweet potato, cassava, alfalfa, lima bean, pea, chick pea, soybean, carrot, strawberry, lettuce, oak, maple, walnut, rose, mint, squash, daisy, quinoa, buckwheat, mung bean, cow pea, lentil, lupin, peanut, fava bean, French beans, mustard, and cactus.

30. The plant of embodiment 29, wherein the plant is soybean.

31. The plant of embodiment 27, wherein the plant is a non-vascular plant selected from the group consisting of moss, liverwort, hornwort, and algae.

32. The plant of embodiment 27, wherein the plant is a vascular plant reproducing from spores.

33. A method for stably expressing a recombinant fusion protein comprising kappa-casein and beta-lactoglobulin in a plant, wherein the kappa-casein is full-length kappa-casein comprising an amino acid sequence having at least 90% sequence identity to SEQ ID NO: 4 or the kappa-casein is para-kappa-casein comprising an amino acid sequence having at least 90% sequence identity to SEQ ID NO: 2 and wherein the beta-lactoglobulin is full-length beta-lactoglobulin comprising an amino acid sequence having at least 90% sequence identity to SEQ ID NO: 10, the method comprising: (i) transforming a plant with a plant transformation vector comprising an expression cassette comprising a nucleic acid molecule encoding the fusion protein; and (ii) growing the transformed plant under conditions wherein the recombinant fusion protein is expressed.

34. The method of embodiment 33, wherein the fusion protein is expressed in an amount of 1% or higher per the total protein weight of the soluble protein extractable from the plant.

35. The method of embodiment 33, wherein the fusion protein is expressed in the plant at a level at least 2-fold higher than kappa-casein expressed individually in a plant.

36. The method of embodiment 33, wherein the fusion protein accumulates in the plant at least 2-fold higher than kappa-casein is expressed without beta-lactoglobulin.

37. A food composition comprising a fusion protein comprising kappa-casein and beta-lactoglobulin, wherein the kappa-casein is full-length kappa-casein comprising an amino acid sequence having at least 90% sequence identity to SEQ ID NO: 4 or the kappa-casein is para-kappa-casein comprising an amino acid sequence having at least 90% sequence identity to SEQ ID NO: 2 and wherein the beta-lactoglobulin is full-length beta-lactoglobulin comprising an amino acid sequence having at least 90% sequence identity to SEQ ID NO: 10.

38. The food composition of embodiment 37, wherein the food composition is selected from the group consisting of cheese and processed cheese products, yogurt and fermented dairy products, directly acidified counterparts of fermented dairy products, cottage cheese dressing, frozen dairy products, frozen desserts, desserts, baked goods, toppings, icings, fillings, low-fat spreads, dairy-based dry mixes, soups, sauces, salad dressing, geriatric nutrition, creams and creamers, analog dairy products, follow-up formula, baby formula, infant formula, milk, dairy beverages, acid dairy drinks, smoothies, milk tea, butter, margarine, butter alternatives, growing up milks, low-lactose products and beverages, medical and clinical nutrition products, protein/nutrition bar applications, sports beverages, confections, meat products, analog meat products, meal replacement beverages, weight management food and beverages, cultured buttermilk, sour cream, yogurt, skyr, leben, lassi, kefir, powder containing a milk protein, and low-lactose products.

### Embodiment Set 3: Recombinant Fusion Protein Comprising Beta-Casein and Beta-Lactoglobulin

1. A recombinant fusion protein, comprising: a) beta-casein; and b) beta-lactoglobulin.

1.1 The recombinant fusion protein of embodiment 1, wherein at least one of the beta-casein, or beta-lactoglobulin lack a bovine secretion signal peptide.

1.2 The recombinant fusion protein of embodiment 1, wherein at least one of the beta-casein, or beta-lactoglobulin is a truncated milk protein, lacking a bovine secretion signal peptide.

2. The recombinant fusion protein of embodiment 1, further comprising a protease cleavage site.

3. The recombinant fusion protein of embodiment 1, further comprising a chymosin cleavage site.

4. A fusion protein, comprising: beta-casein and beta-lactoglobulin, wherein the beta-casein comprises an amino acid sequence having at least 90% sequence identity to SEQ ID NO: 6 and wherein the beta-lactoglobulin comprises an amino acid sequence having at least 90% sequence identity to SEQ ID NO: 10.

5. The fusion protein of embodiment 4, further comprising a protease cleavage site.

6. The fusion protein of embodiment 4, further comprising a chymosin cleavage site.

7. A nucleic acid molecule encoding a fusion protein comprising beta-casein and beta-lactoglobulin, wherein the beta-casein comprises an amino acid sequence having at least 90% sequence identity to SEQ ID NO: 6 and wherein the beta-lactoglobulin comprises an amino acid sequence having at least 90% sequence identity to SEQ ID NO: 10.

8. The nucleic acid molecule of embodiment 7, wherein the nucleic acid sequence is codon optimized for expression in a plant.

9. The nucleic acid molecule of embodiment 8, wherein the plant is a soybean plant.

10. An expression vector comprising the nucleic acid molecule of embodiment 7.

11. A host cell comprising the expression vector of embodiment 10.

12. The host cell of embodiment 11, wherein the host cell is selected from the group consisting of plant cells, bacterial cells, fungal cells, and mammalian cells.

13. The host cell of embodiment 11, wherein the host cell is a plant cell.

14. A plant stably transformed with the nucleic acid molecule of embodiment 7.

14.1 A plant transformed to express the recombinant fusion protein of any one of embodiments 1-1.2, wherein the fusion protein accumulates in the plant at least 2-fold higher than beta-casein expressed without beta-lactoglobulin.

15. The plant of embodiment 14, wherein the plant is a monocot selected from the group consisting of turf grass, maize, rice, oat, wheat, barley, sorghum, orchid, iris, lily, onion, palm, and duckweed.

16. The plant of embodiment 14, wherein the plant is a dicot selected from the group consisting of Arabidopsis, tobacco, tomato, potato, sweet potato, cassava, alfalfa, lima bean, pea, chick pea, soybean, carrot, strawberry, lettuce, oak, maple, walnut, rose, mint squash, daisy, quinoa, buckwheat, mung bean, cow pea, lentil, lupin, peanut, fava bean, French beans, mustard, and cactus.

17. The plant of embodiment 14, wherein the plant is a soybean plant.

18. A food composition, comprising: a fusion protein comprising beta-casein and beta-lactoglobulin.

18.1 The food composition of embodiment 18, wherein at least one of the beta-casein, or beta-lactoglobulin lack a bovine secretion signal peptide.

18.2 The food composition of embodiment 18, wherein at least one of the beta-casein, or beta-lactoglobulin is a truncated milk protein, lacking a bovine secretion signal peptide.

19. The food composition of embodiment 18, wherein the food composition is a solid.

20. The food composition of embodiment 18, wherein the food composition is a liquid.

21. The food composition of embodiment 18, wherein the food composition is a powder.

22. The food composition of embodiment 18, wherein the food composition is selected from the group consisting of: cheese, processed cheese product, yogurt, fermented dairy product, directly acidified counterpart of fermented dairy product, cottage cheese, dressing, frozen dairy product, frozen dessert, dessert, baked good, topping, icing, filling, low-fat spread, dairy-based dry mix, soup, sauce, salad dressing, geriatric nutrition, cream, creamer, analog dairy product, follow-up formula, baby formula, infant formula, milk, dairy beverage, acid dairy drink, smoothie, milk tea, butter, margarine, butter alternative, growing up milk, low-lactose product, low-lactose beverage, medical and clinical nutrition product, protein bar, nutrition bar, sport beverage, confection, meat product, analog meat product, meal replacement beverage, weight management food and beverage, dairy product, cultured buttermilk, sour cream, yogurt, skyr, leben, lassi, kefir, powder containing a milk protein, and low-lactose product.

23. The food composition of embodiment 18, wherein the food composition is a dairy product.

24. The food composition of embodiment 18, wherein the food composition is an analog dairy product.

25. The food composition of embodiment 18, wherein the food composition is a low lactose product.

26. The food composition of embodiment 18, wherein the food composition is a milk.

27. The food composition of embodiment 18, wherein the food composition is a cheese.

28. The food composition of embodiment 18, wherein the food composition is fermented.

### Embodiment Set 4: Seed Processing Composition

1. A seed processing composition, comprising: a) a fusion protein, comprising i) a full-length kappa-casein or para-kappa-casein component; and ii) a beta-lactoglobulin component; and b) plant seed tissue.

1.1 The seed processing composition of embodiment 1, wherein at least one of the kappa-casein, para kappa-casein, or beta-lactoglobulin lack a bovine secretion signal peptide.

1.2 The seed processing composition of embodiment 1, wherein at least one of the kappa-casein, para kappa-casein, or beta-lactoglobulin is a truncated milk protein, lacking a bovine secretion signal peptide.

2. The seed processing composition of embodiment 1, wherein the plant seed tissue is ground.

3. The seed processing composition of embodiment 1, wherein the plant seed tissue is soybean.

4. The seed processing composition of embodiment 1, further comprising at least one member selected from the group consisting of: enzyme, protease, chymosin, extractant, solvent, phenol, buffer, additive, salt, protease inhibitor, peptidase inhibitor, osmolyte, and reducing agent.

5. A food composition comprising the seed processing composition of embodiment 1.

6. A protein concentrate composition, comprising a protein concentrate of a fusion protein comprising i) a full-length kappa-casein or para-kappa-casein component, and ii) a beta-lactoglobulin component.

6.1 The protein concentrate composition of embodiment 6, wherein at least one of the kappa-casein, para kappa-casein, or beta-lactoglobulin lack a bovine secretion signal peptide.

6.2 The protein concentrate composition of embodiment 6, wherein at least one of the kappa-casein, para kappa-casein, or beta-lactoglobulin is a truncated milk protein, lacking a bovine secretion signal peptide.

7. The protein concentrate composition of embodiment 6, wherein there is no plant seed tissue present.

8. The protein concentrate composition of embodiment 6, further comprising at least one member selected from the group consisting of: enzyme, protease, chymosin, extractant, solvent, phenol, buffer, additive, salt, protease inhibitor, peptidase inhibitor, osmolyte, and reducing agent.

9. The protein concentrate composition of embodiment 6, further comprising chymosin.

10. A food composition comprising the protein concentrate composition of embodiment 6.

11. A food composition, comprising: a fusion protein, comprising i) a full-length kappa-casein or para-kappa-casein component, and ii) a beta-lactoglobulin component.

12. The food composition of embodiment 11, wherein the food composition comprises the fusion protein comprising the full-length kappa-casein component and a beta-lactoglobulin component.

13. The food composition of embodiment 11, wherein the food composition comprises the fusion protein comprising the para-kappa-casein component and a beta-lactoglobulin component.

14. The food composition of embodiment 11, wherein the food composition is a solid.

15. The food composition of embodiment 11, wherein the food composition is a liquid.

16. The food composition of embodiment 11, wherein the food composition is a powder.

17. The food composition of embodiment 11, wherein the food composition is selected from the group consisting of: cheese, processed cheese product, fermented dairy product, directly acidified counterpart of fermented dairy product, cottage cheese, dressing, frozen dairy product, frozen dessert, dessert, baked good, topping, icing, filling, low-fat spread, dairy-based dry mix, soup, sauce, salad dressing, geriatric nutrition, cream, creamer, analog dairy product, follow-up formula, baby formula, infant formula, milk, dairy beverage, acid dairy drink, smoothie, milk tea, butter, margarine, butter alternative, growing up milk, low-lactose product, low-lactose beverage, medical and clinical nutrition product, protein bar, nutrition bar, sport beverage, confection, meat product, analog meat product, meal replacement beverage, weight management food and beverage, dairy product, cultured buttermilk, sour cream, yogurt, skyr, leben, lassi, kefir, powder containing a milk protein, and low-lactose product.

18. The food composition of embodiment 11, wherein the food composition is a dairy product.

19. The food composition of embodiment 11, wherein the food composition is an analog dairy product.

20. The food composition of embodiment 11, wherein the food composition is a low lactose product.

21. The food composition of embodiment 11, wherein the food composition is a milk.

22. The food composition of embodiment 11, wherein the food composition is a cheese.

23. The food composition of embodiment 11, wherein the food composition is fermented.

24. A method of making a food composition, comprising: combining a fusion protein, comprising i) a full-length kappa-casein or para-kappa-casein component, and ii) a beta-lactoglobulin component, into a food composition.

25. The method of embodiment 24, wherein the food composition is selected from the group consisting of: cheese, processed cheese product, yogurt, fermented dairy product, directly acidified counterpart of fermented dairy product, cottage cheese, dressing, frozen dairy product, frozen dessert, dessert, baked good, topping, icing, filling, low-fat spread, dairy-based dry mix, soup, sauce, salad dressing, geriatric nutrition, cream, creamer, analog dairy product, follow-up formula, baby formula, infant formula, milk, dairy beverage, acid dairy drink, smoothie, milk tea, butter, margarine, butter alternative, growing up milk, low-lactose product, low-lactose beverage, medical and clinical nutrition product, protein bar, nutrition bar, sport beverage, confection, meat product, analog meat product, meal replacement beverage, weight management food and beverage, dairy product, cultured buttermilk, sour cream, yogurt, skyr, leben, lassi, kefir, powder containing a milk protein, and low-lactose product.

26. The method of embodiment 24, wherein the food composition is a dairy product.

27. The method of embodiment 24, wherein the food composition is a cheese.

28. A food composition made by the method of embodiment 24.

29. A method for making a fusion protein, comprising: (a) transforming a host cell with a vector comprising an expression cassette comprising a nucleic acid molecule encoding the fusion protein, wherein the fusion protein comprises i) a full-length kappa-casein or para-kappa-casein component, and ii) a beta-lactoglobulin component, and (b) growing the transformed host cell under conditions wherein the fusion protein is expressed.

30. The method of embodiment 29, wherein the host cell is selected from the group consisting of plant cells, bacterial cells, fungal cells, and mammalian cells.

31. The method of embodiment 29, wherein the host cell is a plant cell.

32. A fusion protein made by the method of embodiment 29.

### Embodiment Set 5: Transgenic Plant Comprising a Recombinant DNA Encoding a Fusion Protein Comprising Bovine Casein and Bovine Beta-Lactoglobulin

1. A transgenic plant, comprising: a recombinant DNA construct encoding a fusion protein, the fusion protein comprising a bovine casein and bovine beta-lactoglobulin.

1.1 The transgenic plant of embodiment 1, wherein at least one of the beta-casein, or beta-lactoglobulin lack a bovine secretion signal peptide.

1.2 The transgenic plant of embodiment 1, wherein at least one of the beta-casein, or beta-lactoglobulin is a truncated milk protein, lacking a bovine secretion signal peptide.

2. The transgenic plant of embodiment 1, wherein the fusion protein comprises a protease cleavage site.

3. The transgenic plant of embodiment 1, wherein the fusion protein comprises a chymosin cleavage site.

4. The transgenic plant of embodiment 1, wherein the fusion protein has a molecular weight of 30 kDa to 50 kDa.

5. A method of making a food composition, comprising: a) extracting the bovine casein and bovine beta-lactoglobulin fusion protein from the transgenic plant of embodiment 1; b) optionally separating the bovine casein from the bovine beta-lactoglobulin; and c) combining the fusion protein or the bovine casein or the bovine beta-lactoglobulin into a food composition.

6. The method of embodiment 5, wherein the food composition is selected from the group consisting of: cheese, processed cheese product, fermented dairy product, directly acidified counterpart of fermented dairy product, cottage cheese, dressing, frozen dairy product, frozen dessert, dessert, baked good, topping, icing, filling, low-fat spread, dairy-based dry mix, soup, sauce, salad dressing, geriatric nutrition, cream, creamer, analog dairy product, follow-up formula, baby formula, infant formula, milk, dairy beverage, acid dairy drink, smoothie, milk tea, butter, margarine, butter alternative, growing up milk, low-lactose product, low-lactose beverage, medical and clinical nutrition product, protein bar, nutrition bar, sport beverage, confection, meat product, analog meat product, meal replacement beverage, weight management food and beverage, dairy product, cultured buttermilk, sour cream, yogurt, skyr, leben, lassi, kefir, powder containing a milk protein, and low-lactose product.

7. The method of embodiment 5, wherein the bovine casein is not separated from the bovine beta-lactoglobulin and the food composition comprises the fusion protein.

8. The method of embodiment 5, wherein the bovine casein is separated from the bovine beta-lactoglobulin and the food composition comprises the bovine casein.

9. The method of embodiment 5, wherein the bovine casein is separated from the bovine beta-lactoglobulin and the food composition comprises the bovine beta-lactoglobulin.

10. The method of embodiment 5, wherein the food composition is a solid.

11. The method of embodiment 5, wherein the food composition is a liquid.

12. The method of embodiment 5, wherein the food composition is a powder.

13. The method of embodiment 5, wherein the food composition is a dairy product.

14. The method of embodiment 5, wherein the food composition is an analog dairy product.

15. The method of embodiment 5, wherein the food composition is a low lactose product.

16. The method of embodiment 5, wherein the food composition is a milk.

17. The method of embodiment 5, wherein the food composition is a cheese.

### Embodiment Set 6: Recombinant Fusion Protein Comprising Casein and Beta-Lactoglobulin

1. A recombinant fusion protein, comprising: a) casein; and b) beta-lactoglobulin.

1.1 The recombinant fusion protein of embodiment 1, wherein at least one of the casein, or beta-lactoglobulin lack a bovine secretion signal peptide.

1.2 The recombinant fusion protein of embodiment 1, wherein at least one of the casein, or beta-lactoglobulin is a truncated milk protein, lacking a bovine secretion signal peptide.

2. The recombinant fusion protein of embodiment 1, further comprising a protease cleavage site.

3. The recombinant fusion protein of embodiment 1, further comprising a chymosin cleavage site.

4. The recombinant fusion protein of embodiment 1, wherein the casein is bovine.

5. The recombinant fusion protein of embodiment 1, wherein the β-lactoglobulin is bovine.

6. The recombinant fusion protein of embodiment 1, wherein the casein and (3-lactoglobulin are bovine.

7. A nucleic acid molecule encoding the recombinant fusion protein of embodiment 1.

8. The nucleic acid molecule of embodiment 7, wherein the nucleic acid sequence is codon optimized for expression in a plant.

9. The nucleic acid molecule of embodiment 8, wherein the plant is a soybean plant.

10. An expression vector comprising the nucleic acid molecule of embodiment 7.

11. A host cell comprising the expression vector of embodiment 10.

12. The host cell of embodiment 11, wherein the host cell is selected from the group consisting of plant cells, bacterial cells, fungal cells, and mammalian cells.

13. The host cell of embodiment 11, wherein the host cell is a plant cell.

14. A plant stably transformed with the nucleic acid molecule of embodiment 7.

15. The plant of embodiment 14, wherein the plant is a monocot selected from the group consisting of turf grass, maize, rice, oat, wheat, barley, sorghum, orchid, iris, lily, onion, palm, and duckweed.

16. The plant of embodiment 14, wherein the plant is a dicot selected from the group consisting of Arabidopsis, tobacco, tomato, potato, sweet potato, cassava, alfalfa, lima bean, pea, chick pea, soybean, carrot, strawberry, lettuce, oak, maple, walnut, rose, mint, squash, daisy, quinoa, buckwheat, mung bean, cow pea, lentil, lupin, peanut, fava bean, French beans, mustard, and cactus.

17. The plant of embodiment 14, wherein the plant is a soybean plant.

18. A food composition, comprising: a fusion protein comprising casein and (3-lactoglobulin.

19. The food composition of embodiment 18, wherein the food composition is a solid.

20. The food composition of embodiment 18, wherein the food composition is a liquid.

21. The food composition of embodiment 18, wherein the food composition is a powder.

22. The food composition of embodiment 18, wherein the food composition is selected from the group consisting of: cheese, processed cheese product, yogurt, fermented dairy product, directly acidified counterpart of fermented dairy product, cottage cheese, dressing, frozen dairy product, frozen dessert, dessert, baked good, topping, icing, filling, low-fat spread, dairy-based dry mix, soup, sauce, salad dressing, geriatric nutrition, cream, creamer, analog dairy product, follow-up formula, baby formula, infant formula, milk, dairy beverage, acid dairy drink, smoothie, milk tea, butter, margarine, butter alternative, growing up milk, low-lactose product, low-lactose beverage, medical and clinical nutrition product, protein bar, nutrition bar, sport beverage, confection, meat product, analog meat product, meal replacement beverage, weight management food and beverage, dairy product, cultured buttermilk, sour cream, skyr, leben, lassi, kefir, powder containing a milk protein, and low-lactose product.

23. The food composition of embodiment 18, wherein the food composition is a dairy product.

24. The food composition of embodiment 18, wherein the food composition is an analog dairy product.

25. The food composition of embodiment 18, wherein the food composition is a low lactose product.

26. The food composition of embodiment 18, wherein the food composition is a milk.

27. The food composition of embodiment 18, wherein the food composition is a cheese.

28. The food composition of embodiment 18, wherein the food composition is fermented.

### Embodiment Set 7: Food Composition Comprising at Least One Component of a Fusion Protein

1. A food composition, comprising: at least one component of a fusion protein, the fusion protein comprising i) a bovine casein component and ii) a bovine β-lactoglobulin component, wherein the component has been separated from the fusion protein.

1.1 The food composition of embodiment 1, wherein at least one of the casein component, or beta-lactoglobulin component lack a bovine secretion signal peptide.

1.2 The food composition of embodiment 1, wherein at least one of the casein component, or beta-lactoglobulin component is a truncated milk protein, lacking a bovine secretion signal peptide.

2. The food composition of embodiment 1, wherein the food composition comprises the bovine casein component.

3. The food composition of embodiment 1, wherein the food composition comprises the bovine β-lactoglobulin component.

4. The food composition of embodiment 1, wherein the food composition is selected from the group consisting of: cheese, processed cheese product, fermented dairy product, directly acidified counterpart of fermented dairy product, cottage cheese, dressing, frozen dairy product, frozen dessert, dessert, baked good, topping, icing, filling, low-fat spread, dairy-based dry mix, soup, sauce, salad dressing, geriatric nutrition, cream, creamer, analog dairy product, follow-up formula, baby formula, infant formula, milk, dairy beverage, acid dairy drink, smoothie, milk tea, butter, margarine, butter alternative, growing up milk, low-lactose product, low-lactose beverage, medical and clinical nutrition product, protein bar, nutrition bar, sport beverage, confection, meat product, analog meat product, meal replacement beverage, weight management food and beverage, dairy product, cultured buttermilk, sour cream, yogurt, skyr, leben, lassi, kefir, powder containing a milk protein, and low-lactose product.

5. The food composition of embodiment 1, wherein the food composition is a solid.

6. The food composition of embodiment 1, wherein the food composition is a liquid.

7. The food composition of embodiment 1, wherein the food composition is a powder.

8. The food composition of embodiment 1, wherein the food composition is a dairy product.

9. The food composition of embodiment 1, wherein the food composition is an analog dairy product.

10. The food composition of embodiment 1, wherein the food composition is a low lactose product.

11. The food composition of embodiment 1, wherein the food composition is a milk.

12. The food composition of embodiment 1, wherein the food composition is a cheese.

13. The food composition of embodiment 1, wherein the food composition is fermented.

14. A food composition, comprising: a fusion protein comprising bovine casein and bovine β-lactoglobulin.

15. The food composition of embodiment 14, wherein the fusion protein comprises a protease cleavage site.

16. The food composition of embodiment 14, wherein the fusion protein comprises a chymosin cleavage site.

17. The food composition of embodiment 14, wherein the fusion protein has a molecular weight of 30 kDa to 50 kDa.

18. The food composition of embodiment 14, wherein the food composition is selected from the group consisting of: cheese, processed cheese product, fermented dairy product, directly acidified counterpart of fermented dairy product, cottage cheese, dressing, frozen dairy product, frozen dessert, dessert, baked good, topping, icing, filling, low-fat spread, dairy-based dry mix, soup, sauce, salad dressing, geriatric nutrition, cream, creamer, analog dairy product, follow-up formula, baby formula, infant formula, milk, dairy beverage, acid dairy drink, smoothie, milk tea, butter, margarine, butter alternative, growing up milk, low-lactose product, low-lactose beverage, medical and clinical nutrition product, protein bar, nutrition bar, sport beverage, confection, meat product, analog meat product, meal replacement beverage, weight management food and beverage, dairy product, cultured buttermilk, sour cream, yogurt, skyr, leben, lassi, kefir, powder containing a milk protein, and low-lactose product.

19. The food composition of embodiment 14, wherein the food composition is a solid.

20. The food composition of embodiment 14, wherein the food composition is a liquid.

21. The food composition of embodiment 14, wherein the food composition is a powder.

22. The food composition of embodiment 14, wherein the food composition is a dairy product.

23. The food composition of embodiment 14, wherein the food composition is an analog dairy product.

24. The food composition of embodiment 14, wherein the food composition is a low lactose product.

25. The food composition of embodiment 14, wherein the food composition is a milk.

26. The food composition of embodiment 14, wherein the food composition is a cheese.

27. The food composition of embodiment 14, wherein the food composition is fermented.

28. The food composition of embodiment 14, wherein the fusion protein is a plant expressed fusion protein.

29. The food composition of embodiment 14, wherein the fusion protein is a soybean expressed fusion protein.

### Embodiment Set 8: Alternative Diary Food Composition

1. An alternative dairy food composition comprising: i) a recombinant beta-casein protein, and ii) at least one lipid, wherein the alternative dairy food composition does not comprise any other milk proteins; and wherein the recombinant beta-casein protein confers on the alternative dairy food composition one or more characteristics of a dairy food product selected from the group consisting of: taste, aroma, appearance, handling, mouthfeel, density, structure, texture, elasticity, springiness, coagulation, binding, leavening, aeration, foaming, creaminess and emulsification.

1.1 The alternative dairy food composition of embodiment 1, wherein the beta-casein lacks a bovine secretion signal peptide.

1.2 The alternative dairy food composition of embodiment 1, wherein the beta-casein is a truncated milk protein, lacking a bovine secretion signal peptide.

2. The alternative dairy food composition of embodiment 1, wherein the recombinant beta-casein is plant-expressed.

3. The alternative dairy food composition of embodiment 2, wherein the recombinant beta-casein is expressed in a soybean plant.

4. The alternative dairy food composition of embodiment 1, wherein the composition comprises a fusion protein comprising the recombinant beta-casein.

5. The alternative dairy food composition of embodiment 1, wherein the composition is a milk composition, a cream composition, a yogurt composition, an ice cream composition, a frozen custard composition, a frozen dessert composition, a crème fraiche composition, a curd composition, a cottage cheese composition, or a cream cheese composition.

6. The alternative dairy food composition of embodiment 1, wherein the composition comprises at least one salt.

7. The alternative dairy food composition of embodiment 1, wherein the composition comprises calcium.

8. The alternative dairy food composition of embodiment 1, wherein the composition comprises calcium at a concentration of about 0.01% to about 2% by weight.

9. The alternative dairy food composition of embodiment 1, wherein the composition has a pH of about 4 to about 8.

10. The alternative dairy food composition of embodiment 1, wherein the composition comprises a fusion protein comprising the recombinant beta-casein.

### Embodiment Set 9: Alternative Diary Food Composition

1. A cheese composition comprising recombinant casein protein; wherein about 32% to 100% by weight of the total casein protein in the cheese composition is beta-casein; and wherein the cheese composition has the ability to stretch to at least 3 cm in length without breaking, as determined by heating a 100 gram mass of the composition at a temperature of 225° C. for 4 minutes and cooling to about 90° C. and pulling with a fork placed beneath the mass.

1.1 The cheese composition of embodiment 1, wherein the beta-casein lacks a bovine secretion signal peptide.

1.2 The cheese composition of embodiment 1, wherein the beta-casein is a truncated milk protein, lacking a bovine secretion signal peptide.

2. The cheese composition of embodiment 1, wherein the composition does not comprise any casein proteins other than beta-casein.

3. The cheese composition of embodiment 1, wherein the composition comprises at least one additional casein protein.

4. The cheese composition of embodiment 3, wherein at least 80% by weight of the total casein protein in the composition is beta-casein.

5. The cheese composition of embodiment 3, wherein at least 90% by weight of the total casein protein in the composition is beta-casein.

6. The cheese composition of embodiment 3, wherein at least 95% by weight of the total casein protein in the composition is beta-casein.

7. The cheese composition of embodiment 3, wherein the at least one additional casein protein is selected from kappa-casein, para-kappa-casein, beta-casein, alpha-S1-casein and alpha-S2-casein.

8. The cheese composition of embodiment 3, wherein the at least one additional casein protein is kappa-casein.

9. The cheese composition of embodiment 3, wherein the at least one additional casein protein is para-kappa casein.

10. The cheese composition of embodiment 1, wherein the recombinant beta-casein is plant-expressed.

11. The cheese composition of embodiment 10, wherein the recombinant beta-casein is expressed in a soybean plant.

12. The cheese composition of embodiment 3, wherein all caseins in the composition are plant-expressed.

13. The cheese composition of embodiment 1, wherein the recombinant beta-casein protein is derived from a fusion protein.

14. The cheese composition of embodiment 1, wherein the composition does not contain an organoleptically functional amount of beta-lactoglobulin.

15. The cheese composition of embodiment 1, wherein the composition has the ability to stretch to at least 5 cm in length without breaking, as determined by heating a 100 gram mass of the composition at a temperature of 225° C. for 4 minutes and cooling to about 90° C. and pulling with a fork placed beneath the mass.

16. The cheese composition of embodiment 1, wherein the composition has the ability to stretch to at least 3 cm in length without breaking, as determined by heating a 100 gram mass of the composition at a temperature of 225° C. for 4 minutes and cooling to about 90° C. and pulling with a fork placed beneath the mass; and a firmness of at least 150 grams, as determined by compressing a cylindrical-shaped sample of the cheese composition having a height of 3 cm and a diameter of 3 cm to a height of 1.5 cm at 5° C.

17. The cheese composition of embodiment 1, wherein the composition comprises at least one lipid and at least one salt.

18. The cheese composition of embodiment 1, wherein the composition comprises calcium.

19. The cheese composition of embodiment 18, wherein the composition comprises calcium at a concentration of about 0.01% to about 2% by weight.

20. The cheese composition of embodiment 1, wherein the composition has a pH of about 5.2 to about 5.9.

21. The cheese composition of embodiment 1, wherein the composition comprises at least one organoleptic property similar to cheese produced from mammalian milk selected from the group consisting of taste, appearance, mouthfeel, structure, texture, density, elasticity, springiness, coagulation, binding, leavening, aeration, foaming, creaminess, and emulsification.

22. A method of making the cheese composition of embodiment 1, the method comprising expressing the recombinant beta-casein protein in a plant, extracting the beta-casein from the plant, and combining the beta-casein with at least one lipid and/or salt.

23. A cheese composition comprising a recombinant casein protein; wherein about 32% to 100% by weight of the total casein protein in the cheese composition is beta-casein; and wherein the cheese composition has ability to stretch to at least 5 cm in length without breaking, as determined by heating a 100 gram mass of the composition at a temperature of 225° C. for 4 minutes and cooling to about 90° C. and pulling with a fork placed beneath the mass.

24. The cheese composition of embodiment 23, wherein the composition does not comprise any additional casein proteins.

25. The cheese composition of embodiment 23, wherein the composition comprises at least one additional casein protein, and wherein at least 80% by weight of the total casein protein in the composition is beta-casein.

26. The cheese composition of embodiment 25, wherein the at least one additional casein protein is kappa-casein or para-kappa casein.

27. The cheese composition of embodiment 23, wherein the recombinant beta-casein is plant-expressed.

28. The cheese composition of embodiment 23, wherein the recombinant beta-casein protein is derived from a fusion protein.

29. The cheese composition of embodiment 23, wherein the composition has at least one of the following characteristics: i) a firmness of at least 150 grams, as determined by compressing a cylindrical-shaped sample of the cheese composition having a height of 3 cm and a diameter of 3 cm to a height of 1.5 cm at 5° C.; or ii) a melting point of about 35° C. to about 100° C.

30. A method of making the cheese composition of embodiment 23, the method comprising expressing the recombinant beta-casein protein in a plant, extracting the beta-casein from the plant, and combining the beta-casein with at least one lipid and/or salt.

### Embodiment Set 10: Fusion Protein Comprising First and Second Milk Proteins, and Transformed Plants Expressing the Same

1. A transformed plant comprising in its genome: a recombinant DNA construct encoding a fusion protein, the fusion protein comprising a first protein and a second protein, wherein the first protein and/or second protein is a milk protein, and wherein the fusion protein is expressed in the plant in an amount of 1% or higher per total protein weight of soluble protein extractable from the plant.

1.1 The transformed plant of embodiment 1, wherein the first milk protein and/or second milk protein lacks an animal secretion signal peptide.

1.2 The transformed plant of embodiment 1, wherein the first milk protein and/or second milk protein is a truncated milk protein, lacking an animal secretion signal peptide.

1.3 The transformed plant of embodiment 1, wherein the fusion protein is expressed in an amount of 2%, 3%, 4%, 5%, 6%, 7%, 8%, 9%, 10%, 11%, 12%, 13%, 14%, 15%, 16%, 17%, 18%, 19%, 20% or higher per total protein weight of soluble protein extractable from the plant.

1.4 The transformed plant of any one of embodiments 1-1.3, wherein the fusion protein is expressed at least 1, 2, 3, 4, 5, 6, 7, 8, 9, 10, 11, 12, 13, 14, 15, 16, 17, 18, 19, 20, 21, 22, 23, 24, 25, 50, 75, 100, 125, 150, 175, 200 or more fold higher than a control plant expressing only the first or second milk proteins individually.

2. The transformed plant of any one of embodiments 1-1.4, wherein the fusion protein comprises, from N-terminus to C-terminus, the first protein and the second protein.

3. The transformed plant of any one of embodiments 1-1.4, wherein the fusion protein comprises, from N-terminus to C-terminus, the second protein and the first protein.

4. The transformed plant of any one of embodiments 1-3, wherein the milk protein is α-S1 casein, α-S2 casein, β-casein, κ-casein, para-κ-casein, β-lactoglobulin, α-lactalbumin, lysozyme, lactoferrin, lactoperoxidase, or an immunoglobulin.

5. The transformed plant of any one of embodiments 1-3, wherein the milk protein is selected from the group consisting of: SEQ ID NO: 4, or a sequence at least 90% identical thereto; SEQ ID NO: 2, or a sequence at least 90% identical thereto; SEQ ID NO: 6, or a sequence at least 90% identical thereto; SEQ ID NO: 8, or a sequence at least 90% identical thereto; SEQ ID NO: 84, or a sequence at least 90% identical thereto; and SEQ ID NO: 10, or a sequence at least 90% identical thereto.

6. The transformed plant of any one of embodiments 1-5, wherein each of the first protein and the second protein are milk proteins.

7. The transformed plant of any one of embodiments 1-5, wherein the first protein is a milk protein and the second protein is a non-milk protein.

8. The transformed plant of embodiment 7, wherein the non-milk protein is albumin, hemoglobin, collagen, ovalbumin, ovotransferrin, GFP, or ovoglobulin.

9. The transformed plant of embodiment 6, wherein the first protein and the second protein are each casein proteins.

10. The transformed plant of any one of embodiments 1-9, wherein the plant is a dicot.

11. The transformed plant of embodiment 10, wherein the dicot is Arabidopsis, tobacco, tomato, potato, sweet potato, cassava, alfalfa, lima bean, pea, chick pea, soybean, carrot, strawberry, lettuce, oak, maple, walnut, rose, mint, squash, daisy, or cactus.

12. The transformed plant of any one of embodiments 1-9, wherein the plant is soybean.

13. The transformed plant of any one of embodiments 1-12, wherein the fusion protein is stably expressed.

14. The transformed plant of any one of embodiments 1-12, wherein the fusion protein is transiently expressed.

15. The transformed plant of any one of embodiments 1-14, wherein the recombinant DNA construct is codon-optimized for expression in the plant.

16. The transformed plant of any one of embodiments 1-15, wherein the fusion protein comprises a protease cleavage site.

17. The transformed plant of embodiment 16, wherein the protease cleavage site is a chymosin cleavage site.

18. The transformed plant of any one of embodiments 1-17, wherein the fusion protein is expressed at a level at least 2-fold higher than a casein protein expressed individually in a plant.

19. A recombinant fusion protein comprising a first protein and a second protein, wherein at least one of the first protein and the second protein is a milk protein.

19.1 The recombinant fusion protein of embodiment 19, wherein the first milk protein and/or second milk protein lacks an animal secretion signal peptide.

19.2 The recombinant fusion protein of embodiment 19, wherein the first milk protein and/or second milk protein is a truncated milk protein, lacking an animal secretion signal peptide.

20. The recombinant fusion protein of any one of embodiments 19-19.2, wherein the fusion protein comprises, from N-terminus to C-terminus, the first protein and the second protein.

21. The recombinant fusion protein of any one of embodiments 19-19.2, wherein the fusion protein comprises, from N-terminus to C-terminus, the second protein and the first protein.

22. The recombinant fusion protein of any one of embodiments 19-21, wherein the milk protein is α-S1 casein, (α-S2 casein, β-casein, κ-casein, para-κ-casein, β-lactoglobulin, α-lactalbumin, lysozyme, lactoferrin, lactoperoxidase, or an immunoglobulin.

23. The recombinant fusion protein of any one of embodiments 19-21, wherein the milk protein is selected from the group consisting of: SEQ ID NO: 4, or a sequence at least 90% identical thereto; SEQ ID NO: 2, or a sequence at least 90% identical thereto; SEQ ID NO: 6, or a sequence at least 90% identical thereto; SEQ ID NO: 8, or a sequence at least 90% identical thereto; SEQ ID NO: 84, or a sequence at least 90% identical thereto; and SEQ ID NO: 10, or a sequence at least 90% identical thereto.

24. The recombinant fusion protein of any one of embodiments 19-23, wherein the first protein and the second protein are milk proteins.

25. The recombinant fusion protein of any one of embodiments 19-23, wherein the first protein is a milk protein and the second protein is a non-milk protein.

26. The recombinant fusion protein of embodiment 25, wherein the non-milk protein is albumin, hemoglobin, collagen, ovalbumin, ovotransferrin, GFP, or ovoglobulin.

27. The recombinant fusion protein of embodiment 24, wherein the first protein and the second protein are each casein proteins.

28. The recombinant fusion protein of embodiment 27, wherein the first protein and the second protein are the same casein protein.

29. The recombinant fusion protein of embodiment 27, wherein the first protein and the second protein are both α-S1 casein, α-S2 casein, β-casein, κ-casein, or para-κ-casein.

30. The recombinant fusion protein of embodiment 24, wherein the first protein and the second protein are each casein proteins and are different from one another.

31. The recombinant fusion protein of embodiment 30, wherein the first protein and the second protein are each independently selected from α-S1 casein, α-S2 casein, β-casein, κ-casein, and para-κ-casein.

32. A recombinant fusion protein comprising a casein protein and lysozyme, wherein the casein protein is selected from the group consisting of α-S1 casein, α-S2 casein, (3-casein, κ-casein, and para-κ-casein.

33. A recombinant fusion protein comprising a casein protein and β-lactoglobulin, wherein the casein protein is selected from the group consisting of α-S1 casein, α-S2 casein, (3-casein, κ-casein, and para-κ-casein.

34. The recombinant fusion protein of any one of embodiments 19-33, wherein the fusion protein comprises a protease cleavage site.

35. The recombinant fusion protein of embodiment 34, wherein the protease cleavage site is a chymosin cleavage site.

36. A nucleic acid encoding the recombinant fusion protein of any one of embodiments 19-35.

37. The nucleic acid of embodiment 36, wherein the nucleic acid is codon optimized for expression in a plant species.

38. The nucleic of embodiment 36, wherein the nucleic acid is codon optimized for expression in soybean.

39. A vector comprising a nucleic acid encoding a recombinant fusion protein, wherein the recombinant fusion protein comprises a first protein and a second protein, wherein at least one of the first protein and the second protein is a milk protein.

40. The vector of embodiment 39, wherein the vector is a plasmid.

41. The vector of embodiment 40, wherein the vector is an Agrobacterium Ti plasmid.

42. The vector of any one of embodiments 39-41, wherein the nucleic acid comprises, in order from 5′ to 3′: a promoter; a 5′ untranslated region; a sequence encoding the fusion protein of any one of embodiments 19-35; and a terminator.

43. The vector of embodiment 42, wherein the promoter is a seed-specific promoter.

44. The vector of embodiment 43, wherein the seed-specific promoter is selected from the group consisting of PvPhas, BnNap, AtOle1, GmSeed2, GmSeed3, GmSeed5, GmSeed6, GmSeed7, GmSeed8, GmSeed10, GmSeed11, GmSeed12, pBCON, GmCEP1-L, GmTHIC, GmBg7S1, GmGRD, GmOLEA, GmOLER, Gm2S-1, and GmBBld-II.

45. The vector of embodiment 43, wherein the seed-specific promoter is PvPhas and comprises the sequence of SEQ ID NO: 18, or a sequence at least 90% identical thereto.

46. The vector of embodiment 43, wherein the seed-specific promoter is GmSeed2 and comprises the sequence of SEQ ID NO: 19, or a sequence at least 90% identical thereto.

47. The vector of embodiment 42, wherein the 5′ untranslated region is selected from the group consisting of Arc5′UTR and glnB1UTR.

48. The vector of embodiment 47, wherein the 5′ untranslated region is Arc5′UTR and comprises the sequence of SEQ ID NO: 20, or a sequence at least 90% identical thereto.

49. The vector of embodiment 42, wherein the expression cassette comprises a 3′ untranslated region.

50. The vector of embodiment 49, wherein the 3′ untranslated region is Arc5-1 and comprises SEQ ID NO: 21, or a sequence at least 90% identical thereto.

51. The vector of embodiment 42, wherein the terminator sequence is a terminator isolated or derived from a gene encoding Nopaline synthase, Arc5-1, an Extensin, Rb7 matrix attachment region, a Heat shock protein, Ubiquitin 10, Ubiquitin 3, and M6 matrix attachment region.

52. The vector of embodiment 42, wherein the terminator sequence is isolated or derived from a Nopaline synthase gene and comprises the sequence of SEQ ID NO: 22, or a sequence at least 90% identical thereto.

53. The vector of embodiment 42, wherein the terminator sequence is a dual terminator and is selected from the group consisting of: SEQ ID NO: 138, or a sequence at least 90% identical thereto; SEQ ID NO: 141, or a sequence at least 90% identical thereto; SEQ ID NO: 144, or a sequence at least 90% identical thereto; and SEQ ID NO: 146, or a sequence at least 90% identical thereto.

54. A plant-expressed recombinant fusion protein, comprising: κ-casein and (3-lactoglobulin.

55. The plant-expressed recombinant fusion protein of embodiment 54, wherein the fusion protein comprises, in order from N-terminus to C-terminus, the κ-casein and the (3-lactoglobulin.

56. The plant-expressed recombinant fusion protein of embodiment 54 or 55, wherein the fusion protein comprises a protease cleavage site.

57. The plant-expressed recombinant fusion protein of embodiment 56, wherein the protease cleavage site is a chymosin cleavage site.

58. The plant-expressed recombinant fusion protein of any one of embodiments 55-57, wherein the fusion protein comprises a signal peptide.

59. The plant-expressed recombinant fusion protein of embodiment 58, wherein the signal peptide is located at the N-terminus of the fusion protein.

60. The plant-expressed recombinant fusion protein of any one of embodiments 55-59, wherein the fusion protein is encoded by a nucleic acid that is codon optimized for expression in a plant.

61. The plant-expressed recombinant fusion protein of any one of embodiments 55-60, wherein the fusion protein is expressed in a soybean.

62. The plant-expressed recombinant fusion protein of any one of embodiments 55-61, wherein the fusion protein has a molecular weight of 30 kDa to 50 kDa.

63. The plant-expressed recombinant fusion protein of any one of embodiments 55-62, wherein the fusion protein is expressed in a plant in an amount of 1% or higher per total protein weight of soluble protein extractable from the plant.

64. The plant-expressed recombinant fusion protein of any one of embodiments 55-62, wherein the fusion protein is expressed in the plant at a level at least 2-fold higher than κ-casein expressed individually in a plant.

65. The plant-expressed recombinant fusion protein of any one of embodiments 55-62, wherein the fusion protein accumulates in the plant at least 2-fold higher than κ-casein expressed without β-lactoglobulin.

66. A stably transformed plant, comprising in its genome: a recombinant DNA construct encoding a fusion protein, the fusion protein comprising: κ-casein and 3-lactoglobulin; wherein the fusion protein is stably expressed in the plant in an amount of 1% or higher per total protein weight of soluble protein extractable from the plant.

66.1 The stably transformed plant of embodiment 66, wherein the κ-casein and/or β-lactoglobulin protein lacks an animal secretion signal peptide.

66.2 The stably transformed plant of embodiment 66, wherein the κ-casein and/or β-lactoglobulin protein is a truncated milk protein, lacking an animal secretion signal peptide.

66.3 The stably transformed plant of embodiment 66, wherein the fusion protein is expressed in an amount of 2%, 3%, 4%, 5%, 6%, 7%, 8%, 9%, 10%, 11%, 12%, 13%, 14%, 15%, 16%, 17%, 18%, 19%, 20% or higher per total protein weight of soluble protein extractable from the plant.

66.4 The stably transformed plant of any one of embodiments 66-66.3, wherein the fusion protein is expressed at least 1, 2, 3, 4, 5, 6, 7, 8, 9, 10, 11, 12, 13, 14, 15, 16, 17, 18, 19, 20, 21, 22, 23, 24, 25, 50, 75, 100, 125, 150, 175, 200 or more fold higher than a control plant expressing only the κ-casein or β-lactoglobulin protein individually.

67. The stably transformed plant of any one of embodiments 66-66.4, wherein the fusion protein comprises, in order from N-terminus to C-terminus, the κ-casein and the (3-lactoglobulin.

68. The stably transformed plant of any one of embodiments 66-67, wherein the fusion protein comprises a protease cleavage site.

69. The stably transformed plant of embodiment 68, wherein the protease cleavage site is a chymosin cleavage site.

70. The stably transformed plant of any one of embodiments 66-69, wherein the fusion protein comprises a signal peptide.

71. The stably transformed plant of embodiment 70, wherein the signal peptide is located at the N-terminus of the fusion protein.

72. The stably transformed plant of any one of embodiments 66-71, wherein the plant is soybean.

73. The stably transformed plant of any one of embodiments 66-72, wherein the recombinant DNA construct comprises codon-optimized nucleic acids for expression in the plant.

74. The stably transformed plant of any one of embodiments 66-73, wherein the fusion protein has a molecular weight of 30 kDa to 50 kDa.

75. The stably transformed plant of any one of embodiments 66-74, wherein the fusion protein is expressed at a level at least 2-fold higher than κ-casein expressed individually in a plant.

76. The stably transformed plant of any one of embodiments 66-74, wherein the fusion protein accumulates in the plant at least 2-fold higher than κ-casein expressed without β-lactoglobulin.

77. A plant-expressed recombinant fusion protein comprising: a casein protein and β-lactoglobulin.

78. The plant-expressed recombinant fusion protein of embodiment 77, wherein the casein protein is α-S1 casein, α-S2 casein, β-casein, or κ-casein.

79. A stably transformed plant, comprising in its genome: a recombinant DNA construct encoding a fusion protein, the fusion protein comprising: a casein protein and 0-lactoglobulin; wherein the fusion protein is stably expressed in the plant in an amount of 1% or higher per total protein weight of soluble protein extractable from the plant.

79.1 The stably transformed plant of embodiment 79, wherein the casein and/or (3-lactoglobulin protein lacks an animal secretion signal peptide.

79.2 The stably transformed plant of embodiment 79, wherein the casein and/or (3-lactoglobulin protein is a truncated milk protein, lacking an animal secretion signal peptide.

79.3 The stably transformed plant of embodiment 79, wherein the fusion protein is expressed in an amount of 2%, 3%, 4%, 5%, 6%, 7%, 8%, 9%, 10%, 11%, 12%, 13%, 14%, 15%, 16%, 17%, 18%, 19%, 20% or higher per total protein weight of soluble protein extractable from the plant.

79.4 The stably transformed plant of any one of embodiments 79-79.3, wherein the fusion protein is expressed at least 1, 2, 3, 4, 5, 6, 7, 8, 9, 10, 11, 12, 13, 14, 15, 16, 17, 18, 19, 20, 21, 22, 23, 24, 25, 50, 75, 100, 125, 150, 175, 200 or more fold higher than a control plant expressing only the casein or β-lactoglobulin protein individually.

80. The stably transformed plant of any one of embodiments 79-79.4, wherein the casein protein is α-S1 casein, α-S2 casein, β-casein, or κ-casein.

81. A method for stably expressing a recombinant fusion protein in a plant, the method comprising: (a) transforming a plant with a plant transformation vector comprising an expression cassette comprising: a sequence encoding a fusion protein, wherein the fusion protein comprises a first protein and a second protein, wherein at least one of the first protein and the second protein is a milk protein; and (b) growing the transformed plant under conditions wherein the recombinant fusion protein is expressed in an amount of 1% or higher per total protein weight of soluble protein extractable from the plant.

82. The method of embodiment 81, wherein the wherein the milk protein is α-S1 casein, α-S2 casein, β-casein, κ-casein, para-κ-casein, β-lactoglobulin, α-lactalbumin, lysozyme, lactoferrin, lactoperoxidase, or an immunoglobulin.

### Embodiment Set 11: Casein Multimers

1. A fusion protein comprising a first, second, third, and fourth protein, wherein the third protein is kappa-casein.

1.1 A fusion protein comprising a first, second, optionally third, and optionally fourth milk protein.

1.2 The fusion protein of embodiment 1 or 1.1, wherein at least one of the first, second, third or fourth proteins lacks an animal secretion signal peptide.

1.3 The fusion protein of embodiment 1 or 1.1, wherein at least one of the first, second, third or fourth proteins is a truncated milk protein, lacking an animal secretion signal peptide.

1.4 The fusion protein of any one of embodiments 1-1.3, wherein the first, second, third and fourth proteins are selected from the group consisting of α-S1 casein, α-S2 casein, β-casein, κ-casein, para-κ-casein, β-lactoglobulin, α-lactalbumin, lysozyme, lactoferrin, lactoperoxidase, and an immunoglobulin.

2. The fusion protein of embodiment 1 wherein: the first protein beta-casein; the second protein is beta-casein; and the fourth protein is beta-lactoglobulin.

3. The fusion protein of embodiment 1 or 2, wherein the kappa-casein comprises a chymosin cleavage site.

4. The fusion protein of any one of embodiments 1-1.4, wherein cleavage of the fusion protein with chymosin produces the following polypeptides: a first polypeptide comprising the first protein, the second protein, and para-kappa-casein; and a second polypeptide comprising a kappa-casein macropeptide and the fourth protein.

5. A nucleic acid encoding the fusion protein of any one of embodiments 1-4.

6. A transformed plant comprising the fusion protein of any one of embodiments 1-4 or the nucleic acid of embodiment 5.

7. A food composition comprising the fusion protein of any one of embodiments 1-6.

8. A food composition comprising a first, second, third or fourth protein, wherein the first, second, third, our fourth protein is derived from the fusion protein of any one of embodiments 1-7.

9. A method of making a food composition, the method comprising: (i) expressing a fusion protein in a transformed plant; (ii) preparing a food composition comprising the fusion protein and plant protein from the same transformed plant in which the fusion protein was produced.

10. The method of embodiment 9, wherein the transformed plant is soybean.

11. A food composition produced using the method of any one of embodiments 9-10.

### Embodiment Set No. 12: Fusion Protein Comprising Milk Protein and a Fusion Partner

1. A fusion protein comprising a first protein and a second protein, wherein the first protein is a milk protein, and the second protein comprises at least one of the following characteristics: a molecular weight of 15 kDa or higher; at least 30% hydrophobic amino acids; and/or less than about 2.5 disulfide bonds per 10 kDa molecular weight.

2. The fusion protein of embodiment 1, wherein the second protein comprises at least two of the characteristics (i), (ii) and (iii).

3. The fusion protein of embodiment 1, wherein the second protein comprises all three of the characteristics (i), (ii) and (iii).

4. The fusion protein of any one of embodiments 1-3, wherein the fusion protein comprises a protease cleavage site located between the first protein and the second protein.

5. The fusion protein of embodiment 4, wherein the protease cleavage site is a chymosin cleavage site.

6. The fusion protein of embodiment 4 or 5, wherein cleavage of the fusion protein with a protease separates the first protein from the second protein.

7. The fusion protein of embodiment 6, wherein after being separated from one another, the first protein and/or the second protein optionally comprise at their N-terminus or C-terminus one or more amino acids that do not occur in the native form of the first protein or the second protein and that are derived from the protease cleavage site.

8. A nucleic acid encoding the fusion protein of any one of embodiments 1-7.

9. A transformed plant comprising the fusion protein of any one of embodiments 1-7 or the nucleic acid of embodiment 8.

10. A food composition comprising the fusion protein of any one of embodiments 1-7.

### Embodiment Set 13: Co-Expression of a Milk Protein and a Protein Capable of Forming a Protein Body

1. A composition comprising a first vector and a second vector, wherein the first vector comprises a sequence that encodes a milk protein, and the second vector comprises a sequence that encodes a prolamin.

2. A composition comprising a vector, wherein the vector comprises: a first sequence that encodes a milk protein; and a second sequence that encodes a prolamin.

2.1 The composition of embodiment 1 or 2, wherein the milk protein lacks an animal secretion signal peptide.

2.2 The composition of embodiment 1 or 2, wherein the milk proteins is a truncated milk protein, lacking an animal secretion signal peptide.

3. The composition of any one of embodiments 1-2.2, wherein the milk protein is selected from the group consisting of: α-S1 casein, α-S2 casein, β-casein, κ-casein, para-κ-casein, β-lactoglobulin, α-lactalbumin, lysozyme, lactoferrin, lactoperoxidase, and an immunoglobulin.

4. The composition of embodiment 3, wherein the milk protein is the β-casein.

5. The composition of embodiment 3, wherein the milk protein is the (3-lactoglobulin.

6. The composition of any one of embodiments 1-5, wherein the prolamin is selected from the group consisting of: gliadin, a hordein, a secalin, a zein, a kafirin, and an avenin.

7. The composition of embodiment 6, wherein the prolamin is a zein.

8. A plant comprising the composition of any one of embodiments 1-7.

9. A method for stably expressing one or more recombinant proteins in a plant, the method comprising transforming a plant with the composition of any one of embodiments 1-7, thereby stably expressing one or more recombinant proteins in the plant.

10. The method of embodiment 9, wherein the method is effective in: (a) increasing expression of the one or more recombinant proteins in the plant, relative to expression of the milk protein alone, without co-expression of the prolamin; (b) increasing accumulation of the milk protein in the plant, relative to expression of the milk protein alone, without co-expression of the prolamin; or (c) (a) and (b).

11. The method of embodiment 9 or 10, comprising (a), wherein the method is effective in increasing expression of the milk protein by at least about 1-fold, 5-fold, 50-fold, or 100-fold.

12. The method of embodiment 9 or 10, comprising (b), wherein the method is effective in increasing accumulation of the milk protein in the plant by at least about 1-fold, 5-fold, 10-fold, or 50-fold.

13. A food composition that comprises a recombinant protein isolated from the plant of any one of embodiments 9-12.

14. A plant expressing a fusion protein comprising a milk protein and a prolamin.

14.1 The composition of embodiment 14, wherein the milk protein lacks an animal secretion signal peptide.

14.2 The composition of embodiment 1, wherein the milk proteins is a truncated milk protein, lacking an animal secretion signal peptide.

### Embodiment Set 14: Fusion Protein Comprising a Milk Protein and a Protein Capable of Forming a Protein Body

1. A recombinant fusion protein comprising a prolamin protein and a milk protein.

1.1 The recombinant fusion of embodiment 1, wherein the milk protein lacks an animal secretion signal peptide.

1.2 The recombinant fusion of embodiment 1, wherein the milk proteins is a truncated milk protein, lacking an animal secretion signal peptide.

2. The recombinant fusion protein of any one of embodiments 1-1.2, wherein the milk protein is a casein protein.

3. The recombinant fusion protein of embodiment 2, wherein the casein protein is α-S1 casein, (α-S2 casein, β-casein, κ-casein, or para-κ-casein.

4. The recombinant fusion protein of any one of embodiments 1-1.2, wherein the milk protein is β-lactoglobulin, α-lactalbumin, lysozyme, lactoferrin, lactoperoxidase, or an immunoglobulin.

5. The recombinant fusion protein of any one of embodiments 1-4, wherein the prolamin protein is a gliadin, a hordein, a secalin, a zein, a kafirin, or an avenin.

6. The recombinant fusion protein of embodiment 5, wherein the prolamin protein is a zein.

7. The recombinant fusion protein of embodiment 6, wherein the zein has the sequence of any one of SEQ ID NO: 800, 809 or 811, or a sequence at least 90% identical thereto.

8. The recombinant fusion protein of any one of embodiments 1-1.2, wherein the prolamin protein is a canein.

9. The recombinant fusion protein of embodiment 8, wherein the canein has the sequence of any one of SEQ ID NO: 800, 809 or 811, or a sequence at least 90% identical thereto.

10. The recombinant fusion protein of any one of embodiments 1-1.2, wherein the fusion protein has a sequence of SEQ ID NO: 803 or 807, or a sequence at least 90% identical thereto.

11. A nucleic acid encoding the recombinant fusion protein of any one of embodiments 1-10.

12. A transgenic plant comprising the recombinant fusion protein of any one of embodiments 1-10 or the nucleic acid of embodiment 11.

13. The transgenic plant of embodiment 12, wherein the plant is a dicot.

14. The transgenic plant of embodiment 13, wherein the dicot is arabidopsis, tobacco, tomato, potato, sweet potato, cassava, alfalfa, lima bean, pea, chick pea, soybean, carrot, strawberry, lettuce, oak, maple, walnut, rose, mint, squash, daisy, or cactus.

15. The transgenic plant of embodiment 13, wherein dicot is a soybean.

16. A food composition comprising the recombinant fusion protein of any one of embodiments 1-10, or a prolamin protein or milk protein derived therefrom.

17. A protein body comprising a recombinant fusion protein of any one of embodiments 1-10.

18. The protein body of embodiment 17, wherein a transgenic plant comprises the protein body.

19. The protein body of embodiment 18, wherein the transgenic plant is a dicot.

20. The protein body of embodiment 19, wherein the dicot is a soybean.

### Embodiment Set 15: Fusion Protein Comprising an Unstructured Milk Protein and a Structured Protein; Transgenic Plants Expressing the Same

1. A stably transformed plant comprising in its genome: a recombinant DNA construct encoding a fusion protein, the fusion protein comprising: (i) an unstructured milk protein, and (ii) a structured animal protein; wherein the fusion protein is stably expressed in the plant in an amount of 1% or higher per total protein weight of soluble protein extractable from the plant.

1.1 The stably transformed plant of embodiment 1, wherein the unstructured milk protein lacks an animal secretion signal peptide.

1.2 The stably transformed plant of embodiment 1, wherein the unstructured milk proteins is a truncated milk protein, lacking an animal secretion signal peptide.

1.3 The stably transformed plant of any one of embodiments 1-1.2, wherein the fusion protein is expressed in an amount of 2%, 3%, 4%, 5%, 6%, 7%, 8%, 9%, 10%, 11%, 12%, 13%, 14%, 15%, 16%, 17%, 18%, 19%, 20% or higher per total protein weight of soluble protein extractable from the plant.

1.4 The stably transformed plant of any one of embodiments 1-1.3, wherein the fusion protein is expressed at least 1, 2, 3, 4, 5, 6, 7, 8, 9, 10, 11, 12, 13, 14, 15, 16, 17, 18, 19, 20, 21, 22, 23, 24, 25, 50, 75, 100, 125, 150, 175, 200 or more fold higher than a control plant expressing only the unstructured milk protein individually.

2. The stably transformed plant of any one of embodiments 1-1.2, wherein the fusion protein comprises, from N-terminus to C-terminus, the unstructured milk protein and the animal protein.

3. The stably transformed plant of any one of embodiments 1-2, wherein the unstructured milk protein is α-S1 casein, α-S2 casein, β-casein, or κ-casein.

4. The stably transformed plant of embodiment 1, wherein the unstructured milk protein is κ-casein and comprises the sequence of SEQ ID NO: 4, or a sequence at least 90% identical thereto.

5. The stably transformed plant of any one of embodiments 1-1.4, wherein the unstructured milk protein is para-κ-casein and comprises the sequence of SEQ ID NO: 2, or a sequence at least 90% identical thereto.

6. The stably transformed plant of any one of embodiments 1-1.4, wherein the unstructured milk protein is β-casein and comprises the sequence of SEQ ID NO: 6, or a sequence at least 90% identical thereto.

7. The stably transformed plant of any one of embodiments 1-1.4, wherein the unstructured milk protein is α-S1 casein and comprises the sequence SEQ ID NO: 8, or a sequence at least 90% identical thereto.

8. The stably transformed plant of any one of embodiments 1-1.4, wherein the unstructured milk protein is α-S2 casein and comprises the sequence SEQ ID NO: 84, or a sequence at least 90% identical thereto.

9. The stably transformed plant of any one of embodiments 1-8, wherein the structured animal protein is a structured mammalian protein.

10. The stably transformed plant of embodiment 9, wherein the structured mammalian protein is β-lactoglobulin, α-lactalbumin, albumin, lysozyme, lactoferrin, lactoperoxidase, hemoglobin, collagen, or an immunoglobulin.

11. The stably transformed plant of embodiment 9, wherein the structured mammalian protein is β-lactoglobulin and comprises the sequence of SEQ ID NO: 10, or a sequence at least 90% identical thereto.

12. The stably transformed plant of any one of embodiments 1-8, wherein the structured animal protein is a structured avian protein.

13. The stably transformed plant embodiment 12, wherein the structured avian protein is ovalbumin, ovotransferrin, lysozyme or ovoglobulin.

14. The stably transformed plant of embodiment 9, wherein the milk protein is κ-casein and the structured mammalian protein is β-lactoglobulin.

15. The stably transformed plant of embodiment 9, wherein the milk protein is para-κ-casein and the structured mammalian protein is β-lactoglobulin.

16. The stably transformed plant of embodiment 9, wherein the milk protein is (3-casein and the structured mammalian protein is β-lactoglobulin.

17. The stably transformed plant of embodiment 9, wherein the milk protein is α-S1 casein or α-S2 casein and the structured mammalian protein is β-lactoglobulin.

18. The stably transformed plant of any one of embodiments 1-17, wherein the plant is a dicot.

19. The stably transformed plant of embodiment 18, wherein the dicot is Arabidopsis, tobacco, tomato, potato, sweet potato, cassava, alfalfa, lima bean, pea, chick pea, soybean, carrot, strawberry, lettuce, oak, maple, walnut, rose, mint, squash, daisy, Quinoa, buckwheat, mung bean, cow pea, lentil, lupin, peanut, fava bean, French beans (i.e., common beans), mustard, or cactus.

20. The stably transformed plant of any one of embodiments 1-19, wherein the plant is soybean.

21. The stably transformed plant of any one of embodiments 1-20, wherein the recombinant DNA construct is codon-optimized for expression in the plant.

22. The stably transformed plant of any one of embodiments 1-21, wherein the fusion protein comprises a protease cleavage site.

23. The stably transformed plant of embodiment 22, wherein the protease cleavage site is a chymosin cleavage site.

24. The stably transformed plant of any one of embodiments 1-23, wherein the fusion protein is expressed at a level at least 2-fold higher than an unstructured milk protein expressed individually in a plant.

25. The stably transformed plant of any one of embodiments 1-24, wherein the fusion protein accumulates in the plant at least 2-fold higher than an unstructured milk protein expressed without the structured animal protein.

26. A recombinant fusion protein comprising: (i) an unstructured milk protein, and (ii) a structured animal protein.

26.1 The recombinant fusion protein of embodiment 26, wherein the unstructured milk protein lacks an animal secretion signal peptide.

26.2 The recombinant fusion protein of embodiment 26, wherein the unstructured milk protein is a truncated milk protein, lacking an animal secretion signal peptide.

27. The recombinant fusion protein of any one of embodiments 26-26.2, wherein the fusion protein is expressed in a plant.

28. The recombinant fusion protein of any one of embodiments 26-27, wherein the unstructured milk protein is α-S1 casein, α-S2 casein, β-casein, or κ-casein.

29. The recombinant fusion protein of embodiment 28, wherein the milk protein is κ-casein and comprises the sequence of SEQ ID NO: 4, or a sequence at least 90% identical thereto.

30. The recombinant fusion protein of embodiment 28, wherein the milk protein is para-κ-casein and comprises the sequence of SEQ ID NO: 2, or a sequence at least 90% identical thereto.

31. The recombinant fusion protein of embodiment 28, wherein the milk protein is β-casein and comprises the sequence of SEQ ID NO: 6, or a sequence at least 90% identical thereto.

32. The recombinant fusion protein of embodiment 28, wherein the milk protein is α-S1 casein and comprises the sequence SEQ ID NO: 8, or a sequence at least 90% identical thereto.

33. The recombinant fusion protein of embodiment 28, wherein the milk protein is α-S2 casein and comprises the sequence SEQ ID NO: 84, or a sequence at least 90% identical thereto.

34. The recombinant fusion protein of any one of embodiments 26-33, wherein the structured animal protein is a structured mammalian protein.

35. The recombinant fusion protein of embodiment 34, wherein the structured mammalian protein is β-lactoglobulin, α-lactalbumin, albumin, lysozyme, lactoferrin, lactoperoxidase, hemoglobin, collagen, or an immunoglobulin.

36. The recombinant fusion protein of embodiment 34, wherein the structured mammalian protein is β-lactoglobulin and comprises the sequence of SEQ ID NO: 10, or a sequence at least 90% identical thereto.

37. The recombinant fusion protein of any one of embodiments 26-33, wherein the structured animal protein is a structured avian protein.

38. The recombinant fusion protein of embodiment 37, wherein the structured avian protein is ovalbumin, ovotransferrin, lysozyme or ovoglobulin.

39. The recombinant fusion protein embodiment 34, wherein the milk protein is κ-casein and the structured mammalian protein is β-lactoglobulin.

40. The recombinant fusion protein of embodiment 34, wherein the milk protein is para-κ-casein and the structured mammalian protein is β-lactoglobulin.

41. The recombinant fusion protein of embodiment 34, wherein the milk protein is β-casein and the structured mammalian protein is β-lactoglobulin.

42. The recombinant fusion protein of embodiment 34, wherein the milk protein is α-S1 casein or α-S2 casein and the structured mammalian protein is β-lactoglobulin.

43. The recombinant fusion protein of embodiment 34, wherein the fusion protein comprises a protease cleavage site.

44. The recombinant fusion protein of embodiment 34, wherein the protease cleavage site is a chymosin cleavage site.

45. A nucleic acid encoding the recombinant fusion protein of any one of embodiments 26 to 44.

46. The nucleic acid of embodiment 45, wherein the nucleic acid is codon optimized for expression in a plant species.

47. The nucleic of embodiment 45 or 46, wherein the nucleic acid is codon optimized for expression in soybean.

48. A vector comprising a nucleic acid encoding a recombinant fusion protein, wherein the recombinant fusion protein comprises: (i) an unstructured milk protein, and (ii) a structured animal protein.

49. The vector of embodiment 48, wherein the vector is a plasmid.

50. The vector of embodiment 49, wherein the vector is an Agrobacterium Ti plasmid.

51. The vector of any one of embodiments 48-50, wherein the nucleic acid comprises, in order from 5′ to 3′: a promoter; a 5′ untranslated region; a sequence encoding the fusion protein; and a terminator.

52. The vector of embodiment 51, wherein the promoter is a seed-specific promoter.

53. The vector of embodiment 52, wherein the seed-specific promoter is selected from the group consisting of PvPhas, BnNap, AtOle1, GmSeed2, GmSeed3, GmSeed5, GmSeed6, GmSeed7, GmSeed8, GmSeed10, GmSeed11, GmSeed12, pBCON, GmCEP1-L, GmTHIC, GmBg7S1, GmGRD, GmOLEA, GmOLER, Gm2S-1, and GmBBld-II.

54. The vector of embodiment 53, wherein the seed-specific promoter is PvPhas and comprises the sequence of SEQ ID NO: 18, or a sequence at least 90% identical thereto.

55. The vector of embodiment 53, wherein the seed-specific promoter is GmSeed2 and comprises the sequence of SEQ ID NO: 19, or a sequence at least 90% identical thereto.

56. The vector of any one of embodiments 51-55, wherein the 5′ untranslated region is selected from the group consisting of Arc5′UTR and glnB1UTR.

57. The vector of embodiment 56, wherein the 5′ untranslated region is Arc5′UTR and comprises the sequence of SEQ ID NO: 20, or a sequence at least 90% identical thereto.

58. The vector of any one of embodiments 51-57, wherein the expression cassette comprises a 3′ untranslated region.

59. The vector of embodiment 58, wherein the 3′ untranslated region is Arc5-1 and comprises SEQ ID NO: 21, or a sequence at least 90% identical thereto.

60. The vector of any one of embodiments 51-59, wherein the terminator sequence is a terminator isolated or derived from a gene encoding Nopaline synthase, Arc5-1, an Extensin, Rb7 matrix attachment region, a Heat shock protein, Ubiquitin 10, Ubiquitin 3, and M6 matrix attachment region.

61. The vector of embodiment 60, wherein the terminator sequence is isolated or derived from a Nopaline synthase gene and comprises the sequence of SEQ ID NO: 22, or a sequence at least 90% identical thereto.

62. A plant comprising the recombinant fusion protein of any one of embodiments 26-44 or the nucleic acid of any one of embodiments 45-47.

63. A method for stably expressing a recombinant fusion protein in a plant, the method comprising: a) transforming a plant with a plant transformation vector comprising an expression cassette comprising: a sequence encoding a fusion protein, wherein the fusion protein comprises an unstructured milk protein, and a structured animal protein; and b) growing the transformed plant under conditions wherein the recombinant fusion protein is expressed in an amount of 1% or higher per total protein weight of soluble protein extractable from the plant.

64. The method of embodiment 63, wherein the unstructured milk protein is κ-casein.

65. The method of embodiment 63 or 64, wherein the structured animal protein is β-lactoglobulin.

66. A food composition comprising the recombinant fusion protein of any one of embodiments 26-44.

67. A method for making a food composition, the method comprising: expressing the recombinant fusion protein of any one of embodiments 26-44 in a plant; extracting the recombinant fusion protein from the plant; optionally, separating the milk protein from the structured animal protein or the structured plant protein; and creating a food composition using the milk protein or the fusion protein.

68. The method of embodiment 67, wherein the plant stably expresses the recombinant fusion protein.

69. The method of embodiment 68, wherein the plant expresses the recombinant fusion protein in an amount of 1% or higher per total protein weight of soluble protein extractable from the plant.

70. The method of any one of embodiments 67-69, wherein the plant is soybean.

71. The method of any one of embodiments 67-70, wherein the food composition comprises the structured animal or plant protein.

72. The method of any one of embodiments 67-71, wherein the milk protein and the structured animal or plant protein are separated from one another in the plant cell, prior to extraction.

73. The method of any one of embodiments 67-71, wherein the milk protein is separated from the structured animal or plant protein after extraction, by contacting the fusion protein with an enzyme that cleaves the fusion protein.

74. A food composition produced using the method of any one of embodiments 67-73.

### Embodiment Set Number 16: Modulation of Post-Translational Modifications by Modifying the Amino Acid Sequence of a Milk Protein

1. A recombinant milk protein, wherein the amino acid sequence of the milk protein is modified to promote addition of one or more post-translational modifications in a plant cell.

2. The recombinant milk protein of embodiment 1, wherein the milk protein is expressed in a plant, and wherein the milk protein comprises one or more post-translational modifications that are not present in a non-modified milk protein expressed in the same type of plant.

3. The recombinant milk protein of embodiment 1, wherein the milk protein is expressed in a plant in an amount of 1% or higher per total protein weight of soluble protein extractable from the plant.

4. The recombinant milk protein of any one of embodiments 1-3, wherein the milk protein is a casein protein selected from α-S1 casein, α-S2 casein, β-casein, κ-casein, and para-κ-casein.

5. The recombinant milk protein of any one of embodiments 1-4, wherein the milk protein is κ-casein or para-k-casein.

6. The recombinant milk protein of any one of embodiments 1-4, wherein the milk protein is β-casein.

7. The recombinant milk protein of any one of embodiments 1-4, wherein the milk protein is β-lactoglobulin.

8. The recombinant milk protein of any one of embodiments 1-7, wherein the one or more post-translational modifications are selected from glycosylation, phosphorylation, lipidation, ubiquitylation, nitrosylation, methylation, acetylation, amidation, prenylation, alkylation, gamma-carboxylation, biotinylation, oxidation, and sulfation.

9. A nucleic acid encoding the recombinant milk protein of any one of embodiments 1-8.

### Embodiment Set Number 17: Modulation of Post-Translational Modifications (PTMs) by Expressing One or More Enzymes which Add/Remove PTMs

1. A method for stably expressing a milk protein in a plant, the method comprising: transforming the plant with a sequence encoding the milk protein and a sequence encoding a kinase.

1.1 The method of embodiment 1, wherein the milk protein lacks an animal secretion signal peptide.

1.2 The method of embodiment 1, wherein the milk protein is a truncated milk protein, lacking an animal secretion signal peptide.

2. The method of any one of embodiments 1-1.2, wherein the milk protein is a casein protein selected from the group consisting of: α-S1 casein, α-S2 casein, β-casein, κ-casein, para-κ-casein.

3. The method of any one of embodiments 1-2, wherein the milk protein is fused to a second protein.

4. The method of any one of embodiments 1-3, wherein the kinase is a kinase in the 20 C family.

4.1 The method of any one of embodiments 1-3, wherein the kinase is a kinase in the Casein Kinase 1 (CK1).

4.2 The method of any one of embodiments 1-3, wherein the kinase is a kinase in the Casein Kinase 2 (CK2).

4.3 The method of any one of embodiments 1-3, wherein the kinase is a kinase in the FAMK-1.

4.4 The method of any one of embodiments 1-3, wherein the kinase is a kinase in the Early Flowering1 (EL1)-like proteins.

5. The method of any one of embodiments 1-3, wherein the kinase that phosphorylates Ser-X-Glu/pSer motifs.

5.1 The method of any one of embodiments 1-3, wherein the kinase that phosphorylates pS/pT-X-X-S/T, or (E,D)n-X-X-S/T motifs.

5.2 The method of any one of embodiments 1-3, wherein the kinase that phosphorylates S/T-X-X-E/D/pS/pY motifs.

5.3 The method of any one of embodiments 1-3, wherein the kinase that phosphorylates S-X-S/pE motifs.

6. The method of any one of embodiments 1-3, wherein the kinase is a Fam20C kinase, or a fragment or variant thereof.

6.1 The method of any one of embodiments 1-3, wherein the kinase is a Casein Kinase 1 (CK1), or a fragment or variant thereof.

6.2 The method of any one of embodiments 1-3, wherein the kinase is a Casein Kinase 2 (CK2), or a fragment or variant thereof.

6.3 The method of any one of embodiments 1-3, wherein the kinase is a FAMK-1, or a fragment or variant thereof.

6.1 The method of any one of embodiments 1-3, wherein the kinase is a Early Flowering1 (EL1)-like proteins, or a fragment or variant thereof.

7. The method of any one of embodiments 1-3, wherein the kinase comprises SEQ ID NO: 821, or a sequence at least 90% or 95% identical thereto.

8. The method of any one of embodiments 1-3, wherein the kinase comprises amino acids 94-586 of SEQ ID NO: 821, or a sequence at least 90% or 95% identical thereto.

9. The method of any one of embodiments 1-8, wherein the sequence encoding the milk protein and the sequence encoding the kinase are in the same vector.

10. The method of embodiment 9, wherein the vector is a binary vector.

11. A stably transformed plant expressing a milk protein and a heterologous kinase.

11.1 The stably transformed plant of embodiment 11, wherein the milk protein lacks an animal secretion signal peptide.

11.2 The stably transformed plant of embodiment 11, wherein the milk protein is a truncated milk protein, lacking an animal secretion signal peptide.

12. The stably transformed plant of any one of embodiments 11-11.2, wherein the milk protein is a casein protein selected from the group consisting of: α-S1 casein, α-S2 casein, β-casein, κ-casein, para-κ-casein.

13. The stably transformed plant of any one of embodiments 11-12, wherein the milk protein is fused to a second protein.

14. The stably transformed plant of any one of embodiments 11-13, wherein the kinase is a kinase in the 20C family.

14.1 The stably transformed plant of any one of embodiments 11-13, wherein the kinase is a kinase in the Casein Kinase 1 (CK1).

14.2 The stably transformed plant of any one of embodiments 11-13, wherein the kinase is a kinase in the Casein Kinase 2 (CK2).

14.3 The stably transformed plant of any one of embodiments 11-13, wherein the kinase is a kinase in the FAMK-1.

14.4 The stably transformed plant of any one of embodiments 11-13, wherein the kinase is a kinase in the Early Flowering1 (EL1)-like proteins.

15. The stably transformed plant of any one of embodiments 11-13, wherein the kinase that phosphorylates Ser-X-Glu/pSer motifs.

15.1 The stably transformed plant of any one of embodiments 11-13, wherein the kinase that phosphorylates pS/pT-X-X-S/T, or (E,D)n-X-X-S/T motifs.

15.2 The stably transformed plant of any one of embodiments 11-13, wherein the kinase that phosphorylates S/T-X-X-E/D/pS/pY motifs.

15.3 The stably transformed plant of any one of embodiments 11-13, wherein the kinase that phosphorylates S—X-S/pE motifs.

16. The stably transformed plant of any one of embodiments 11-13, wherein the kinase is a Fam20C kinase, or a fragment or variant thereof.

16.1 The stably transformed plant of any one of embodiments 11-13, wherein the kinase is a Casein Kinase 1 (CK1), or a fragment or variant thereof.

16.2 The stably transformed plant of any one of embodiments 11-13, wherein the kinase is a Casein Kinase 2 (CK2), or a fragment or variant thereof.

16.3 The stably transformed plant of embodiments 11-13, wherein the kinase is a FAMK-1, or a fragment or variant thereof.

16.4 The stably transformed plant of any one of embodiments 11-13, wherein the kinase is an Early Flowering1 (EL1)-like proteins, or a fragment or variant thereof.

17. The stably transformed plant of any one of embodiments 11-13, wherein the kinase comprises SEQ ID NO: 821, or a sequence at least 90% or 95% identical thereto.

18. The stably transformed plant of any one of any one of embodiments 11-13, wherein the kinase comprises amino acids 94-586 of SEQ ID NO: 821, or a sequence at least 90% or 95% identical thereto.

19. The stably transformed plant of any one of embodiments 11-18, wherein the milk protein is expressed in an amount of %, 3%, 4%, 5%, 6%, 7%, 8%, 9%, 10%, 11%, 12%, 13%, 14%, 15%, 16%, 17%, 18%, 19%, 20% or higher per total protein weight of soluble protein extractable from the plant.

20. The stably transformed plant of any one of embodiments 11-19, wherein the milk protein is expressed at least 1, 2, 3, 4, 5, 6, 7, 8, 9, 10, 11, 12, 13, 14, 15, 16, 17, 18, 19, 20, 21, 22, 23, 24, 25, 50, 75, 100, 125, 150, 175, 200 or more fold higher than a control plant expressing only the milk protein without the heterologous kinase.

### Embodiment Set Number 18: Fusion of a Milk Protein to a Glycoprotein Tag

1. A method for stably expressing a milk protein in a plant, the method comprising: transforming the plant with a sequence encoding a milk protein fused to a glycoprotein tag.

1.1 The method of embodiment 1, wherein the milk protein lacks an animal secretion signal peptide.

1.2 The method of embodiment 1, wherein the milk protein is a truncated milk protein, lacking an animal secretion signal peptide.

2. The method of any one of embodiments 1-1.2, wherein the milk protein is a casein protein selected from the group consisting of: α-S1 casein, α-S2 casein, β-casein, κ-casein, para-κ-casein.

3. The method of any one of embodiments 1-2, wherein the milk protein is fused to a second protein.

4. The method of any one of any one of embodiments 1-3, wherein the glycoprotein tag is isolated or derived from a hydroxyproline (Hyp)-rich glycoprotein (GRGP).

5. The method of any one of embodiments 1-3, wherein the glycoprotein tag comprises the M domain of CD45.

6. The method of any one of embodiments 1-3, wherein the glycoprotein tag is an (SP)11 tag.

6.1 The method of any one of embodiments 1-3, wherein the glycoprotein tag is an (SP)11 tag.

6.2 The method of any one of embodiments 1-3, wherein the glycoprotein tag is an (SP)10 tag.

6.3 The method of any one of embodiments 1-3, wherein the glycoprotein tag is an (SP)20 tag.

6.4 The method of any one of embodiments 1-3, wherein the glycoprotein tag is an (SP)32 tag.

6.5 The method of any one of embodiments 1-3, wherein the glycoprotein tag is an (SP)18 tag.

6.6 The method of any one of embodiments 1-3, wherein the glycoprotein tag is a rabies glycoprotein (rgp) tag.

6.7 The method of any one of embodiments 1-3, wherein the glycoprotein tag is an ANITVNITV tag.

6.8 The method of any one of embodiments 1-3, wherein the glycoprotein tag is a CD45 tag.

7. The method of any one of embodiments 1-3, wherein the glycoprotein tag comprises SEQ ID NO: 825, or a sequence at least 90% or 95% identical thereto.

8. The method of any one of embodiments 1-3, wherein the glycoprotein tag comprises SEQ ID NO: 827, or a sequence at least 90% or 95% identical thereto.

9. The method of any one of embodiments 1-8, wherein the sequence encoding the milk protein and the sequence encoding the kinase are in the same vector.

10. The method of embodiment 9, wherein the vector is a binary vector.

11. A stably transformed plant comprising: a milk protein fused to a glycoprotein tag.

11.1 The stably transformed plant of embodiment 11, wherein the milk protein lacks an animal secretion signal peptide.

11.2 The stably transformed plant of embodiment 11, wherein the milk protein is a truncated milk protein, lacking an animal secretion signal peptide.

12. The stably transformed plant of embodiments 11-11.2, wherein the milk protein is a casein protein selected from the group consisting of: α-S1 casein, α-S2 casein, β-casein, κ-casein, para-κ-casein.

13. The stably transformed plant of embodiments 11-12, wherein the milk protein is fused to a second protein.

14. The stably transformed plant of any one of embodiments 11-13, wherein the glycoprotein tag is isolated or derived from a hydroxyproline (Hyp)-rich glycoprotein (GRGP).

15. The stably transformed plant of embodiments 11-13, wherein the glycoprotein tag comprises the M domain of CD45.

16. The stably transformed plant of embodiments 11-13, wherein the glycoprotein tag is an (SP)11 tag.

16.1 The stably transformed plant of embodiments 11-13, wherein the glycoprotein tag is an (SP)11 tag.

16.2 The stably transformed plant of embodiments 11-13, wherein the glycoprotein tag is an (SP)10 tag.

16.3 The stably transformed plant of embodiments 11-13, wherein the glycoprotein tag is an (SP)20 tag.

16.4 The stably transformed plant of embodiments 11-13, wherein the glycoprotein tag is an (SP)32 tag.

16.5 The stably transformed plant of any one of embodiments 11-13, wherein the glycoprotein tag is an (SP)18 tag.

16.6 The stably transformed plant of any one of embodiments 11-13, wherein the glycoprotein tag is a rabies glycoprotein (rgp) tag.

16.7 The stably transformed plant of any one of embodiments 11-13, wherein the glycoprotein tag is an ANITVNITV tag.

16.7 The stably transformed plant of any one of embodiments 11-13, wherein the glycoprotein tag is a CD45 tag.

17. The stably transformed plant of any one of embodiments 11-13, wherein the glycoprotein tag comprises SEQ ID NO: 825, or a sequence at least 90% or 95% identical thereto.

18. The stably transformed plant of any one of embodiments 11-13, wherein the glycoprotein tag comprises SEQ ID NO: 827, or a sequence at least 90% or 95% identical thereto.

19. The stably transformed plant of any one of embodiments 11-18, wherein the milk protein is expressed in an amount of %, 3%, 4%, 5%, 6%, 7%, 8%, 9%, 10%, 11%, 12%, 13%, 14%, 15%, 16%, 17%, 18%, 19%, 20% or higher per total protein weight of soluble protein extractable from the plant.

20. The stably transformed plant of any one of embodiments 11-19, wherein the milk protein is expressed at least 1, 2, 3, 4, 5, 6, 7, 8, 9, 10, 11, 12, 13, 14, 15, 16, 17, 18, 19, 20, 21, 22, 23, 24, 25, 50, 75, 100, 125, 150, 175, 200 or more fold higher than a control plant expressing only the milk protein without the glycoprotein tag.

### Embodiment Set Number 19: Reducing the Expression or Activity of One or More Proteases in a Plant Cell

1. A plant cell for expressing recombinant milk proteins, wherein expression of one or more proteases is knocked down or knocked out, or has reduced activity in the cell.

2. The plant cell of embodiment 1, wherein expression of the one or more proteases is knocked down or knocked out using a gene editing technology or base editing technology.

2.1 The plant cell of embodiment 1, wherein expression of the one or more proteases is knocked down or knocked out by deleting the whole or a portion of the gene encoding the protease.

3. The plant cell of embodiment 1, wherein expression of the one or more proteases is knocked down or knocked out using RNA interference.

3.1 The plant cell of embodiment 1, wherein the protease activity is reduced by: (i) partial or complete deletion of the gene coding for the protease, or (ii) expression of a non-functional protease gene, or (iii) inhibition or reduction of the activity of the expressed protease protein by insertion, substitution or point mutation in the gene coding for the protease.

4. The plant cell of any one of embodiments 1-3.1, wherein the one or more proteases is a cysteine protease, a serine protease, or an aspartyl protease.

4.1 The plant cell of any one of embodiments 1-4, wherein the plant cell expresses a milk protein.

4.2 The plant cell of any one of embodiments 1-4, wherein the plant cell expresses a milk protein selected from the group consisting of: α-S1 casein, α-S2 casein, β-casein, κ-casein, para-κ-casein, β-lactoglobulin, α-lactalbumin, lysozyme, lactoferrin, lactoperoxidase, and an immunoglobulin.

4.3 The plant cell of any one of embodiments 1-4.2, wherein the milk protein lacks an animal secretion signal peptide.

4.4 The plant cell of any one of embodiments 1-4.2, wherein the milk protein is a truncated milk protein, lacking an animal secretion signal peptide.

4.5 The plant cell of any one of embodiments 1-4.4, wherein the protease is selected from among the proteases listed in Table 60 “Exemplary proteases and inhibitors of the same.”

4.6 The plant cell of any one of embodiments 1-4.4, wherein the protease comprises a sequence selected from the protease sequences listed in Table 60 “Exemplary proteases and inhibitors of the same.”.

4.7 The plant cell of any one of embodiments 1-4.4, wherein the protease comprises a sequence selected from the group consisting of SEQ ID No: 919, 920, 922, 924, 926, 928, 930, 932, and 934.

4.8 The plant cell of any one of embodiments 1-4.8, wherein the milk protein is expressed within a fusion protein.

5. A transgenic plant comprising the plant cell of any one of embodiments 1-4.8.

5.1 The transgenic plant of embodiment 5, wherein the milk protein is expressed in an amount of 2%, 3%, 4%, 5%, 6%, 7%, 8%, 9%, 10%, 11%, 12%, 13%, 14%, 15%, 16%, 17%, 18%, 19%, 20% or higher per total protein weight of soluble protein extractable from the plant.

5.2 The transgenic plant of any one of embodiments 5-5.1, wherein the milk protein is expressed at least 1, 2, 3, 4, 5, 6, 7, 8, 9, 10, 11, 12, 13, 14, 15, 16, 17, 18, 19, 20, 21, 22, 23, 24, 25, 50, 75, 100, 125, 150, 175, 200 or more fold higher than a control plant expressing only the unstructured milk protein without the protease knocked down or knocked out, or with reduced activity.

6. A method for stably expressing a recombinant milk protein in a plant, the method comprising: (i) reducing expression of one or more proteases in the plant, (ii) transforming the plant with a plant transformation vector comprising an expression cassette encoding a recombinant milk protein or the fusion protein comprising the recombinant milk protein, (iii) growing the transformed plant under conditions wherein the recombinant milk protein is expressed in an amount of 1% or higher per total weight of soluble protein extractable from the plant.

7. A plant cell expressing a milk protein and a heterologous protease inhibitor.

8. The plant cell of embodiment 7, wherein the plant cell expresses a milk protein selected from the group consisting of: α-S1 casein, α-S2 casein, β-casein, κ-casein, para-κ-casein, β-lactoglobulin, α-lactalbumin, lysozyme, lactoferrin, lactoperoxidase, and an immunoglobulin.

9 The plant cell of any one of embodiments 7-8, wherein the milk protein lacks an animal secretion signal peptide.

10. The plant cell of any one of embodiments 7-9, wherein the milk protein is a truncated milk protein, lacking an animal secretion signal peptide.

11. The plant cell of any one of embodiments 7-10, wherein the milk protein is expressed within a fusion protein.

12. The plant cell of any one of embodiments 7-11, wherein the protease inhibitor is selected from the group consisting of a cysteine protease inhibitor, an aspartic protease inhibitor, a Trypsin-chymotrypsin inhibitor, and a trypsin inhibitor.

13. The plant cell of any one of embodiments 7-12, wherein the protease inhibitor is selected from the group consisting of SICYS8, CID, BB, and API5, and KTi3.

14 The plant cell of any one of embodiments 7-13, wherein the protease inhibitor comprises a sequence selected from the group consisting of SEQ ID No: 936, 938, 940, 942, 944.

15. A transgenic plant comprising the plant cell of any one of embodiments 7-14.

15.1 A transgenic plant comprising expressing the milk protein and the protease knockout or protease inhibitor expression of Table 61.

16. The transgenic plant of embodiment 15 or 15.1, wherein the milk protein is expressed in an amount of 2%, 3%, 4%, 5%, 6%, 7%, 8%, 9%, 10%, 11%, 12%, 13%, 14%, 15%, 16%, 17%, 18%, 19%, 20% or higher per total protein weight of soluble protein extractable from the plant.

17. The transgenic plant of any one of embodiments 15-16, wherein the milk protein is expressed at least 1, 2, 3, 4, 5, 6, 7, 8, 9, 10, 11, 12, 13, 14, 15, 16, 17, 18, 19, 20, 21, 22, 23, 24, 25, 50, 75, 100, 125, 150, 175, 200 or more fold higher than a control plant expressing only the unstructured milk protein without the heterologous protease inhibitor.

### Embodiment Set Number 20: Food Composition Comprising a Milk Protein Derived from a Fusion Protein

1. A food composition comprising the recombinant milk protein derived from a milk protein (e.g., fusion protein) of any one of the embodiment sets above (i.e., embodiment sets 1-20).

2. A method for making a food composition, the method comprising: expressing the milk protein (e.g. recombinant fusion protein) of any one of the embodiment sets above (i.e., embodiment sets 1-20); extracting the recombinant fusion protein from the plant; optionally, separating the first protein from the second protein; and creating a food composition using the separated milk protein or the fusion protein.

3. The method of embodiment 2, wherein the plant stably expresses the recombinant fusion protein.

4. The method of embodiment 2 or 3, wherein the plant expresses the recombinant fusion protein in an amount of 1% or higher per total protein weight of soluble protein extractable from the plant.

5. The method of any one of embodiments 2-4, wherein the plant is soybean.

6. The method of any one of embodiments 2-5, wherein the food composition comprises the first protein and the second protein.

7. The method of embodiment 6, wherein the first protein and the second protein are separated from one another in the plant cell, prior to extraction.

8. The method of embodiment 6, wherein the first protein and the second protein are separated after extraction, by contacting the fusion protein with an enzyme that cleaves the fusion protein.

9. A food composition produced using the method of any one of embodiments 2-8.

10. A food composition comprising a first or second protein, wherein the first or second protein is derived from the fusion protein of any one of the embodiment sets above.

### Embodiment Set Number 21: A Solid-Phase Protein Stabilized-Emulsion Comprising a Recombinant Casein Protein

1. A solid phase, protein-stabilized emulsion comprising at least one recombinant casein protein selected from kappa-casein, para-kappa-casein, beta-casein, alpha-S1-casein and alpha-S2-casein; wherein the emulsion has at least one of the following characteristics: a firmness of at least 150 grams, as determined by compressing a cylindrical-shaped sample of the emulsion having a height of 3 cm and a diameter of 3 cm to a height of 1.5 cm at 5° C.; a melting point of about 35° C. to about 100° C.; or ability to stretch to at least 3 cm in length without breaking, as determined by heating a 100 gram mass of the emulsion at a temperature of about 225° C. for 4 minutes and cooling to about 90° C. and pulling with a fork placed beneath the mass.

2. The solid phase, protein-stabilized emulsion of embodiment 1, wherein the recombinant casein protein is plant-expressed.

3. The solid phase, protein stabilized emulsion of embodiment 1, wherein the recombinant casein protein is yeast-expressed or bacterial-expressed.

4. The solid phase, protein stabilized emulsion of any one of embodiments 1-3, wherein the recombinant casein protein is derived from a fusion protein.

5. The solid phase, protein stabilized emulsion of embodiment 4, wherein the fusion protein comprises a first and a second protein.

6. The solid phase, protein stabilized emulsion of embodiment 5, wherein the first protein comprises β-Casein and the second protein comprises a milk protein.

7. The solid phase, protein stabilized emulsion of embodiment 5, wherein the first protein comprises β-Casein and the second protein comprises a non-milk protein.

8. The solid phase, protein stabilized emulsion of embodiment 6, wherein the milk protein is selected from the group consisting of β-lactoglobulin, casein, α-lactalbumin, lysozyme, lactoferrin, lactoperoxidase, and immunoglobulin.

9. The solid phase, protein stabilized emulsion of embodiment 8, wherein the milk protein is β-lactoglobulin.

10. The solid phase, protein stabilized emulsion of embodiment 8, wherein the milk protein is casein, and wherein the casein is selected from the group consisting of: α-S1 Casein, α-S2 Casein, β-Casein, κ-Casein, and para-κ-Casein.

11. The solid phase, protein stabilized emulsion of embodiment 10, wherein the milk protein is β-Casein.

12. The solid phase, protein-stabilized emulsion of any one of embodiments 1-11, wherein the emulsion comprises at least one lipid and at least one salt.

13. The solid phase, protein-stabilized emulsion of any one of embodiments 1-5, wherein the emulsion comprises at least two plant-expressed casein proteins each selected from kappa-casein, para-kappa-casein, beta-casein, alpha-S1-casein and alpha-S2-casein.

14. The solid phase, protein-stabilized emulsion of any one of embodiments 1-5, wherein the emulsion comprises at least three plant-expressed casein proteins each selected from kappa-casein, para-kappa-casein, beta-casein, alpha-S1-casein and alpha-S2-casein.

15. The solid phase, protein-stabilized emulsion of any one of embodiments 1-14, wherein the emulsion comprises at least one additional mammalian or plant protein that is not a casein protein.

16. The solid phase, protein-stabilized emulsion of embodiment 2, wherein the plant-expressed casein protein is expressed in a soybean plant.

17. The solid phase, protein-stabilized emulsion of any one of embodiments 1-16, wherein the emulsion has a pH of about 5.2 to about 5.9.

18. The solid phase, protein-stabilized emulsion of any one of embodiments 1-17, wherein the emulsion does not contain an organoleptically functional amount of beta-lactoglobulin.

19. A solid phase, protein-stabilized emulsion comprising one plant-expressed casein protein selected from kappa-casein, para-kappa-casein, beta-casein, alpha-S1-casein, and alpha-S2-casein; wherein the emulsion does not contain any additional casein proteins; wherein the emulsion has at least one of the following characteristics: a firmness of at least 150 grams, as determined by compressing a cylindrical-shaped sample of the emulsion having a height of 3 cm and a diameter of 3 cm to a height of 1.5 cm at 5° C.; a melting point of about 35° C. to about 100° C.; or ability to stretch to at least 3 cm in length without breaking, as determined by heating a 100 gram mass of the emulsion at a temperature of about 225° C. for 4 minutes and cooling to about 90° C. and pulling with a fork placed beneath the mass.

20. The solid phase, protein-stabilized emulsion of embodiment 19, wherein the emulsion further comprises at least one lipid and at least one salt.

21. The solid phase, protein-stabilized emulsion of embodiment 19 or 20, wherein the plant-expressed casein protein is expressed in soybean plant.

22. The solid phase, protein-stabilized emulsion of any one of embodiments 19-21, wherein the plant-expressed casein protein is derived from a fusion protein.

23. The solid phase, protein stabilized-emulsion of any one of embodiments 19-22, wherein the emulsion has a pH of about 5.2 to about 5.9.

24. The solid phase, protein-stabilized emulsion of any one of embodiments 19-23, wherein the emulsion does not contain an organoleptically functional amount of beta-lactoglobulin.

25. A solid phase, protein-stabilized emulsion comprising: a plant-expressed casein protein selected from kappa-casein, para-kappa-casein, beta-casein, alpha-S1-casein, and alpha-S2-casein; and plant-expressed beta-lactoglobulin; wherein the ratio of the casein protein to the beta-lactoglobulin is about 8:1 to about 1:2.

26. The solid phase, protein-stabilized emulsion of embodiment 25, wherein the emulsion has at least one of the following characteristics: a firmness of at least 150 grams, as determined by compressing a cylindrical-shaped sample of the emulsion having a height of 3 cm and a diameter of 3 cm to a height of 1.5 cm at 5° C.; a melting point of about 35° C. to about 100° C.; or ability to stretch to at least 3 cm in length without breaking, as determined by heating a 100 gram mass of the emulsion at a temperature of about 225° C. for 4 minutes and cooling to about 90° C. and pulling with a fork placed beneath the mass.

27. The solid phase, protein-stabilized emulsion of embodiment 25 or 26, wherein the emulsion comprises at least at least one additional mammalian or plant protein that is not a casein protein.

28. The solid phase, protein-stabilized emulsion of any one of embodiments 25-27, wherein the ratio of the casein protein to the beta-lactoglobulin is about 2:1.

29. The solid phase, protein-stabilized emulsion of any one of embodiments 25-28, wherein the emulsion has a pH of about 5.2 to about 5.9.

30. The solid phase, protein-stabilized emulsion of any one of embodiments 25-29, wherein the plant-expressed casein protein is derived from a fusion protein.

31. A solid-phase protein-stabilized emulsion comprising about 8% (w/v) to about 25% (w/v) total protein, one or more lipids, and one or more salts; wherein at least 4% of the total protein comprises casein proteins selected from kappa-casein, para-kappa-casein, beta-casein, alpha-S1-casein, and alpha-S2-casein; wherein at least 20% to 100% of the casein protein is kappa casein; wherein the emulsion has at least one of the following characteristics: a firmness of at least 150 grams, as determined by compressing a cylindrical-shaped sample of the emulsion having a height of 3 cm and a diameter of 3 cm to a height of 1.5 cm at 5° C.; a melting point of about 35° C. to about 100° C.; or ability to stretch to at least 3 cm in length without breaking, as determined by heating a 100 gram mass of the emulsion at a temperature of about 225° C. for 4 minutes and cooling to about 90° C. and pulling with a fork placed beneath the mass.

32. The solid-phase protein-stabilized emulsion of embodiment 31, wherein the kappa casein is expressed in a plant.

33. The solid phase, protein-stabilized emulsion of any one of embodiments 31-32, wherein the kappa casein is derived from a fusion protein.

34. The solid phase, protein-stabilized emulsion of any one of embodiments 31-33, wherein the emulsion has a pH of about 5.2 to about 5.9.

35. The solid phase, protein-stabilized emulsion of any one of embodiments 31-34, wherein the composition comprises only one, only two, only three, or only four casein proteins selected from kappa-casein, para-kappa-casein, beta-casein, alpha-S1-casein, and alpha-S2-casein.

36. The solid phase, protein-stabilized emulsion of any one of embodiments 31-35, wherein the emulsion does not contain an organoleptically functional amount of beta-lactoglobulin.

37. A solid-phase protein-stabilized emulsion comprising about 8% to about 25% total protein, one or more lipids, and one or more salts; wherein at least 4% of the total protein comprises casein proteins selected from kappa-casein, para-kappa-casein, beta-casein, alpha-S1-casein, and alpha-S2-casein; wherein at least 20% to 100% of the casein protein is para-kappa casein; wherein the emulsion has at least one of the following characteristics: a firmness of at least 150 grams, as determined by compressing a cylindrical-shaped sample of the emulsion having a height of 3 cm and a diameter of 3 cm to a height of 1.5 cm at 5° C.; a melting point of about 35° C. to about 100° C.; or ability to stretch to at least 3 cm in length without breaking, as determined by heating a 100 gram mass of the emulsion at a temperature of about 225° C. for 4 minutes and cooling to about 90° C. and pulling with a fork placed beneath the mass.

38. The solid-phase protein-stabilized emulsion of embodiment 37, wherein the para-kappa casein is expressed in a plant.

39. The solid phase, protein-stabilized emulsion of embodiment 37 or 38, wherein the para-kappa casein is derived from a fusion protein.

40. The solid-phase protein-stabilized emulsion of any one of embodiments 37-39 wherein the para-kappa casein is produced without the use of any enzyme that cleaves kappa-casein to para-kappa casein.

41. The solid phase, protein-stabilized emulsion of any one of embodiments 37-40, wherein the emulsion has a pH of about 5.2 to about 5.9.

42. The solid phase, protein-stabilized emulsion of any one of embodiments 37-41, wherein the composition comprises only one, only two, only three, or only four casein proteins selected from kappa-casein, para-kappa-casein, beta-casein, alpha-S1-casein, and alpha-S2-casein.

43. The solid phase, protein-stabilized emulsion of any one of embodiments 37-42, wherein the emulsion does not contain an organoleptically functional amount of beta-lactoglobulin.

44. A solid-phase protein-stabilized emulsion comprising about 8% to about 25% total protein, one or more lipids, and one or more salts; wherein at least 4% of the total protein comprises casein proteins selected from kappa-casein, para-kappa-casein, beta-casein, alpha-ST-casein, and alpha-S2-casein; wherein at least 50% to 100% of the casein protein is beta-casein; wherein the emulsion has at least one of the following characteristics: a firmness of at least 150 grams, as determined by compressing a cylindrical-shaped sample of the emulsion having a height of 3 cm and a diameter of 3 cm to a height of 1.5 cm at 5° C.; a melting point of about 35° C. to about 100° C.; or ability to stretch to at least 3 cm in length without breaking, as determined by heating a 100 gram mass of the emulsion at a temperature of about 225° C. for 4 minutes and cooling to about 90° C. and pulling with a fork placed beneath the mass.

45. The solid-phase protein-stabilized emulsion of embodiment 44, wherein the beta-casein is expressed in a plant.

46. The solid phase, protein-stabilized emulsion of any one of embodiments 44-45, wherein the plant-expressed casein protein is derived from a fusion protein.

47. The solid phase, protein-stabilized emulsion of any one of embodiments 44-46, wherein the emulsion has a pH of about 5.2 to about 5.9.

48. The solid phase, protein-stabilized emulsion of any one of embodiments 44-47, wherein the composition comprises only one, only two, only three, or only four casein proteins selected from kappa-casein, para-kappa-casein, beta-casein, alpha-S1-casein, and alpha-S2-casein.

49. The solid phase, protein-stabilized emulsion of any one of embodiments 44-48, wherein the emulsion does not contain an organoleptically functional amount of beta-lactoglobulin.

50. A solid-phase protein-stabilized emulsion comprising about 8% to about 25% total protein, one or more lipids, and one or more salts; wherein at least 4% of the total protein comprises casein proteins selected from kappa-casein, para-kappa-casein, beta-casein, alpha-S1-casein, and alpha-S2-casein; wherein at least 50% to 100% of the casein protein is alpha-S1-casein; wherein the emulsion has at least one of the following characteristics: a firmness of at least 150 grams, as determined by compressing a cylindrical-shaped sample of the emulsion having a height of 3 cm and a diameter of 3 cm to a height of 1.5 cm at 5° C.; a melting point of about 35° C. to about 100° C.; or ability to stretch to at least 3 cm in length without breaking, as determined by heating a 100 gram mass of the emulsion at a temperature of about 225° C. for 4 minutes and cooling to about 90° C. and pulling with a fork placed beneath the mass.

51. The solid-phase protein-stabilized emulsion of embodiment 50, wherein the alpha-S1-casein is expressed in a plant.

52. The solid phase, protein-stabilized emulsion of any one of embodiments 50-51, wherein the alpha-S1-casein is derived from a fusion protein.

53. The solid phase, protein-stabilized emulsion of any one of embodiments 50-52, wherein the emulsion has a pH of about 5.2 to about 5.9.

54. The solid phase, protein-stabilized emulsion of any one of embodiments 50-53, wherein the composition comprises only one, only two, only three, or only four casein proteins selected from kappa-casein, para-kappa-casein, beta-casein, alpha-S1-casein, and alpha-S2-casein.

55. The solid phase, protein-stabilized emulsion of any one of embodiments 50-54, wherein the emulsion does not contain an organoleptically functional amount of beta-lactoglobulin.

56. A solid-phase protein-stabilized emulsion comprising about 8% to about 25% total protein, one or more lipids, and one or more salts; wherein at least 4% of the total protein comprises casein proteins selected from kappa-casein, para-kappa-casein, beta-casein, alpha-S1-casein, and alpha-S2-casein; wherein at least 20% to 100% of the casein protein is alpha-S2-casein; wherein the emulsion has at least one of the following characteristics: a firmness of at least 150 grams, as determined by compressing a cylindrical-shaped sample of the emulsion having a height of 3 cm and a diameter of 3 cm to a height of 1.5 cm at 5° C.; a melting point of about 35° C. to about 100° C.; or ability to stretch to at least 3 cm in length without breaking, as determined by heating a 100 gram mass of the emulsion at a temperature of about 225° C. for 4 minutes and cooling to about 90° C. and pulling with a fork placed beneath the mass.

57. The solid-phase protein-stabilized emulsion of embodiment 56, wherein the alpha-S2-casein is expressed in a plant.

58. The solid phase, protein-stabilized emulsion of any one of embodiments 56-57, wherein the plant-expressed casein protein is derived from a fusion protein.

59. The solid phase, protein-stabilized emulsion of any one of embodiments 56-58, wherein the emulsion has a pH of about 5.2 to about 5.9.

60. The solid phase, protein-stabilized emulsion of any one of embodiments 56-59, wherein the composition comprises only one, only two, only three, or only four casein proteins selected from kappa-casein, para-kappa-casein, beta-casein, alpha-S1-casein, and alpha-S2-casein.

61. The solid phase, protein-stabilized emulsion of any one of embodiments 56-60, wherein the emulsion does not contain an organoleptically functional amount of beta-lactoglobulin.

### Embodiment Set Number 22: Alternative Dairy Compositions Comprising One or More Recombinant Casein Proteins

1. An alternative dairy composition comprising one or more recombinant casein proteins selected from kappa-casein, para-kappa-casein, beta-casein, alpha-S1-casein, and alpha-S2-casein; wherein the alternative dairy composition has at least one of the following characteristics: a firmness of at least 150 grams, as determined by compressing a cylindrical-shaped sample of the alternative dairy composition having a height of 3 cm and a diameter of 3 cm to a height of 1.5 cm at 5° C.; a melting point of about 35° C. to about 100° C.; or ability to stretch to at least 3 cm in length without breaking, as determined by heating a 100 gram mass of the alternative diary composition at a temperature of about 225° C. for 4 minutes and cooling to about 90° C. and pulling with a fork placed beneath the mass.

2. The alternative dairy composition of embodiment 1, wherein the composition further comprises at least one lipid and at least one salt.

3. The alternative dairy composition of embodiment any one of embodiments 1-2, wherein the composition further comprises at least one additional mammalian or plant protein that is not a casein protein.

4. The alternative dairy composition of any one of embodiments 1-3, wherein the one or more recombinant casein proteins are expressed in a plant.

5. The alternative dairy composition of embodiment 4, wherein the one or more recombinant casein proteins are expressed in a soybean plant.

6. The alternative diary composition of any one of embodiments any one of embodiments 1-5, wherein the one or more recombinant casein proteins are derived from one or more fusion proteins.

7. The alternative diary composition of embodiment 6, wherein one of the one or more fusion proteins comprises a first and a second protein.

8. The alternative diary composition of embodiment 7, wherein the first protein comprises β-Casein and the second protein comprises a milk protein.

9. The alternative diary composition of embodiment 7, wherein the first protein comprises β-Casein and the second protein comprises a non-milk protein.

10. The alternative diary composition of embodiment 8, wherein the milk protein is selected from the group consisting of β-lactoglobulin, casein, α-lactalbumin, lysozyme, lactoferrin, lactoperoxidase, and immunoglobulin.

11. The alternative diary composition of embodiment 8, wherein the milk protein is β-lactoglobulin.

12. The alternative diary composition of embodiment 8, wherein the milk protein is casein, and wherein the casein is selected from the group consisting of: α-S1 Casein, α-S2 Casein, β-Casein, κ-Casein, and para-κ-Casein.

13. The alternative diary composition of embodiment 8, wherein the milk protein is β-Casein.

14. The alternative dairy composition of any one of embodiments 1-13, wherein the composition has a pH of about 5.2 to about 5.9.

15. The alternative dairy composition of any one of embodiments 1-9, wherein the composition does not contain an organoleptically functional amount of beta-lactoglobulin.

16. An alternative dairy composition comprising one or more recombinant casein proteins, one or more lipids; and one or more salts; wherein the alternative dairy composition does not contain an organoleptically functional amount of beta-lactoglobulin; wherein the alternative dairy composition has at least one of the following characteristics: a firmness of at least 150 grams, as determined by compressing a cylindrical-shaped sample of the alternative dairy composition having a height of 3 cm and a diameter of 3 cm to a height of 1.5 cm at 5° C.; a melting point of about 35° C. to about 100° C.; or ability to stretch to at least 3 cm in length without breaking, as determined by heating a 100 gram mass of the alternative diary composition at a temperature of about 225° C. for 4 minutes and cooling to about 90° C. and pulling with a fork placed beneath the mass.

17. The alternative dairy composition of embodiment 16, wherein the composition comprises at least one additional mammalian or plant protein that is not a casein protein.

18. The alternative dairy composition of embodiment 16 or 17, wherein the one or more recombinant casein proteins are expressed in a plant.

19. The alternative dairy composition of claim 18, wherein the one or more recombinant casein proteins are expressed in a soybean plant.

20. The alternative diary composition of any one of embodiments 16-19, wherein the one or more recombinant casein proteins are derived from one or more fusion proteins.

21. The alternative dairy composition of any one of embodiments 16-20, wherein the composition has a pH of about 5.2 to about 5.9.

22. An alternative dairy composition comprising: a recombinant casein protein selected from kappa-casein, para-kappa-casein, beta-casein, alpha-S1-casein, and alpha-S2-casein; and a recombinant beta-lactoglobulin; wherein the ratio of the casein protein to the beta-lactoglobulin is about 8:1 to about 1:2; wherein the alternative dairy composition has at least one of the following characteristics: a firmness of at least 150 grams, as determined by compressing a cylindrical-shaped sample of the alternative dairy composition having a height of 3 cm and a diameter of 3 cm to a height of 1.5 cm at 5° C.; a melting point of about 35° C. to about 100° C.; or ability to stretch to at least 3 cm in length without breaking, as determined by heating a 100 gram mass of the alternative dairy composition at a temperature of about 225° C. for 4 minutes and cooling to about 90° C. and pulling with a fork placed beneath the mass.

23. The alternative dairy composition of embodiment 22, wherein the composition comprises at least one additional mammalian or plant protein that is not a casein protein.

24. The alternative dairy composition of embodiment 22 or 23, wherein recombinant casein protein is expressed in a plant.

25. The alternative dairy composition of claim 24, wherein recombinant casein protein is expressed in a soybean plant.

26. The alternative dairy composition of any one of embodiments 22-25, wherein the recombinant casein protein is derived from a fusion protein.

27. The alternative dairy composition of any one of embodiments 22-26, wherein the composition has a pH of about 5.2 to about 5.9.

28. An alternative dairy composition comprising kappa-casein and essentially no para-kappa casein, wherein the alternative dairy composition has at least one of the following characteristics: a firmness of at least 150 grams, as determined by compressing a cylindrical-shaped sample of the alternative dairy composition having a height of 3 cm and a diameter of 3 cm to a height of 1.5 cm at 5° C.; a melting point of about 35° C. to about 100° C.; or ability to stretch to at least 3 cm in length without breaking, as determined by heating a 100 gram mass of the alternative diary composition at a temperature of about 225° C. for 4 minutes and cooling to about 90° C. and pulling with a fork placed beneath the mass.

29. The alternative dairy composition of embodiment 28, wherein the composition comprises at least one additional mammalian or plant protein that is not a casein protein.

30. The alternative dairy composition of embodiment 28 or 29, wherein the kappa casein is recombinant.

31. The alternative dairy composition of any one of embodiments 28-30, wherein the kappa casein is expressed in a plant.

32. The alternative dairy composition of embodiment 31, wherein the kappa casein is expressed in a soybean plant.

33. The alternative diary composition of any one of embodiments 28-32, wherein the kappa casein is derived from a fusion protein.

34. The alternative dairy composition of any one of embodiments 28-33, wherein the composition has a pH of about 5.2 to about 5.9.

35. The alternative dairy composition of any one of embodiments 28-33, wherein the composition does not contain an organoleptically functional amount of beta-lactoglobulin.

36. An alternative dairy composition comprising one to four of the milk proteins selected from kappa-casein, para-kappa-casein, beta-casein, alpha-S1-casein, and alpha-S2-casein; wherein the alternative dairy composition has at least one of the following characteristics: a firmness of at least 150 grams, as determined by compressing a cylindrical-shaped sample of the alternative dairy composition having a height of 3 cm and a diameter of 3 cm to a height of 1.5 cm at 5° C.; a melting point of about 35° C. to about 100° C.; or ability to stretch to at least 3 cm in length without breaking, as determined by heating a 100 gram mass of the alternative dairy composition at a temperature of about 225° C. for 4 minutes and cooling to about 90° C. and pulling with a fork placed beneath the mass.

37. The alternative dairy composition of embodiment 36, wherein at least one milk protein is recombinant.

38. The alternative dairy composition of embodiment 36, wherein the at least one milk protein is plant-expressed.

39. The alternative dairy composition of embodiment 38, wherein the at least one milk protein is expressed in a soybean plant.

40. The alternative dairy composition of embodiment 37, wherein the at least one milk protein is yeast- or bacterial-expressed.

41. The alternative diary composition of any one of embodiments 36-40, wherein at least one milk protein is derived from a fusion protein.

42. The alternative dairy composition of any one of embodiments 36-41, wherein the alternative dairy composition comprises one of the milk proteins selected from kappa-casein, para-kappa-casein, beta-casein, alpha-S1-casein, and alpha-S2-casein.

43. The alternative dairy composition of any one of embodiments 36-41, wherein the alternative dairy composition comprises two of the milk proteins selected from kappa-casein, para-kappa-casein, beta-casein, alpha-S1-casein, and alpha-S2-casein.

44. The alternative dairy composition of any one of embodiments 36-41, wherein the alternative dairy composition comprises three of the milk proteins selected from kappa-casein, para-kappa-casein, beta-casein, alpha-S1-casein, and alpha-S2-casein.

45. The alternative dairy composition of any one of embodiments 36-41, wherein the alternative dairy composition comprises four of the milk proteins selected from kappa-casein, para-kappa-casein, beta-casein, alpha-S1-casein, and alpha-S2-casein.

46. The alternative dairy composition of any one of embodiments 36-45, wherein the composition has a pH of about 5.2 to about 5.9.

47. The alternative dairy composition of any one of embodiments 36-46, wherein the composition does not contain an organoleptically functional amount of beta-lactoglobulin.

48. An alternative dairy composition comprising 2 to 4 casein proteins; wherein the alternative dairy composition has at least one of the following characteristics: a firmness of at least 150 grams, as determined by compressing a cylindrical-shaped sample of the alternative dairy composition having a height of 3 cm and a diameter of 3 cm to a height of 1.5 cm at 5° C.; a melting point of about 35° C. to about 100° C.; or ability to stretch to at least 3 cm in length without breaking, as determined by heating a 100 gram mass of the alternative dairy composition at a temperature of about 225° C. for 4 minutes and cooling to about 90° C. and pulling with a fork placed beneath the mass.

49. The alternative dairy composition of embodiment 48, wherein the alternative dairy composition does not contain an organoleptically functional amount of beta-lactoglobulin.

50. The alternative dairy composition of embodiment 48 or 49, wherein the casein proteins are selected from kappa-casein, para-kappa-casein, beta-casein, alpha-S1-casein, and alpha-S2-casein.

51. The alternative dairy composition of any one of embodiments 48-50, wherein the composition comprises at least one lipid and at least one salt.

52. The alternative dairy composition of any one of embodiments 48-51, wherein the composition has a pH of about 5.2 to about 5.9.

53. The alternative diary composition of any one of embodiments 48-52, wherein at least one of the casein proteins is derived from a fusion protein.

54. An alternative dairy composition comprising one to four plant-expressed recombinant milk proteins, wherein the alternative dairy composition comprises three or more organoleptic properties similar to a dairy composition selected from the group consisting of taste, appearance, mouthfeel, structure, texture, density, elasticity, springiness, coagulation, binding, leavening, aeration, foaming, creaminess, and emulsification.

55. The alternative dairy composition of embodiment 54, wherein the plant-expressed milk proteins are selected from beta lactoglobulin, kappa-casein, para-kappa-casein, beta-casein, alpha-S1-casein, and alpha-S2-casein.

56. The alternative dairy composition of embodiment 54 or 55, wherein the composition is a milk composition.

57. The alternative dairy composition of embodiment 54 or 55, wherein the composition is a cream composition.

58. The alternative dairy composition of embodiment 54 or 55, wherein the composition is a yogurt composition.

59. The alternative dairy composition of embodiment 54 or 55, wherein the composition is an ice cream composition.

60. The alternative dairy composition of embodiment 54 or 55, wherein the composition is a frozen custard composition.

61. The alternative dairy composition of embodiment 54 or 55, wherein the composition is a frozen desert composition.

62. The alternative dairy composition of embodiment 54 or 55, wherein the composition is a crème fraiche composition.

63. The alternative dairy composition of embodiment 54 or 55, wherein the composition is a curd composition.

64. The alternative dairy composition of embodiment 54 or 55, wherein the composition is a cottage cheese composition.

65. The alternative dairy composition of embodiment 54 or 55, wherein the composition is a cream cheese composition.

66. The alternative dairy composition of any one of embodiments 54-65, wherein at least one of the plant-expressed recombinant milk proteins is derived from a fusion protein.

67. An alternative dairy food composition comprising: a recombinant beta-casein protein, and least one lipid, wherein the alternative dairy food composition does not comprise an organoleptically functional amount of beta-lactoglobulin.

68. The alternative dairy food composition of embodiment 67, wherein the recombinant beta-casein protein confers on the alternative dairy food composition one or more characteristics of a dairy food product selected from the group consisting of: taste, aroma, appearance, handling, mouthfeel, density, structure, texture, elasticity, springiness, coagulation, binding, leavening, aeration, foaming, creaminess and emulsification.

69. The alternative dairy food composition of embodiment 67 or 68, wherein the composition does not comprise any additional casein proteins.

70. The alternative dairy food composition of embodiment 67 or 68, wherein the composition comprises at least one additional casein protein.

71. The alternative dairy food composition of embodiment 70, wherein at least 50% by weight of the total casein protein in the composition is beta-casein.

72. The alternative dairy food composition of embodiment 70, wherein at least 75% by weight of the total casein protein in the composition is beta-casein.

73. The alternative dairy food composition of embodiment 70, wherein at least 90% by weight of the total casein protein in the composition is beta-casein.

74. The alternative dairy food composition of any one of embodiments 70-73, wherein the at least one additional casein protein is selected from kappa-casein, para-kappa-casein, beta-casein, alpha-S1-casein and alpha-S2-casein.

75. The alternative dairy food composition of any one embodiments 70-73, wherein the at least one additional casein protein is kappa-casein or para-kappa casein.

76. The alternative dairy food composition of any one of embodiments 67-75, wherein the recombinant beta-casein is plant-expressed.

77. The alternative dairy food composition of embodiment 76, wherein the recombinant beta-casein is expressed in a soybean.

78. The alternative dairy food composition of any one of embodiments 70-77, wherein all caseins in the composition are plant-expressed.

79. The alternative dairy food composition of any one of embodiments 67-78, wherein the composition comprises a fusion protein comprising the recombinant beta-casein.

80. The alternative dairy food composition any one of embodiments 67-79, wherein the recombinant beta-casein protein confers on the alternative dairy food composition two or more characteristics of a dairy food product selected from the group consisting of: taste, aroma, appearance, handling, mouthfeel, density, structure, texture, elasticity, springiness, coagulation, binding, leavening, aeration, foaming, creaminess and emulsification.

81. The alternative dairy food composition of any one of embodiments 67-80, wherein the composition is a milk composition, a cream composition, a yogurt composition, an ice cream composition, a frozen custard composition, a frozen dessert composition, a crème fraiche composition, a curd composition, a cottage cheese composition, or a cream cheese composition.

82. The alternative dairy food composition of any one of embodiments 67-81, wherein the composition comprises at least one lipid and at least one salt.

83. The alternative dairy food composition any one of embodiments 67-82, wherein the composition comprises calcium.

84. The alternative dairy food composition of embodiment 83, wherein the composition comprises calcium at a concentration of about 0.1% to about 2% by weight.

85. The alternative dairy food composition any one of embodiments 67-84, wherein the composition has a pH of about 4 to about 8.

### Embodiment Set Number 23: Colloidal Suspensions Comprising One or More Recombinant Casein Proteins

1. A colloidal suspension comprising: one to four plant-expressed recombinant milk proteins, wherein the recombinant milk proteins comprise between 0.5% (w/v) to 15% (w/v) of the composition; and ash; wherein the colloidal suspension has at least one, at least two, or at least three characteristics that are substantially similar to bovine milk selected from taste, appearance, mouthfeel, structure, texture, density, elasticity, springiness, coagulation, binding, leavening, aeration, foaming, creaminess, and emulsification.

2. The colloidal suspension of embodiment 1, wherein the plant-expressed milk proteins are selected from beta-lactoglobulin, kappa-casein, para-kappa-casein, beta-casein, alpha-S1-casein, and alpha-S2-casein.

3. The colloidal suspension of any one of embodiments 1-2, wherein at least one of the plant-expressed recombinant milk proteins is derived from a fusion protein.

4. A colloidal suspension comprising: one casein protein, wherein the casein protein comprises between 0.5% (w/v) to 15% (w/v); and ash; wherein the colloidal suspension has at least one, at least two, or at least three characteristics that are substantially similar to bovine milk selected from taste, appearance, mouthfeel, structure, texture, density, elasticity, springiness, coagulation, binding, leavening, aeration, foaming, creaminess, and emulsification.

5. The colloidal suspension of embodiment 4, wherein the casein protein is selected from beta lactoglobulin, kappa-casein, para-kappa-casein, beta-casein, alpha-S1-casein, and alpha-S2-casein.

6. The colloidal suspension of embodiment 4 or 5, wherein the casein protein is beta-casein.

7. The colloidal suspension of any one of embodiments 4-6, wherein the casein protein is plant-expressed.

8. The colloidal suspension of any one of embodiments 4-7, wherein the casein protein is derived from a fusion protein.

9. A method of making an alternative dairy composition comprising processing the colloidal suspension of any one of embodiments 1-8.

10. An alternative dairy composition produced from the method of embodiment 9.

11. The alternative dairy composition of embodiment 10, wherein the alternative dairy composition is a cream composition, a yogurt composition, a cheese composition, an ice cream composition, a frozen custard composition, a frozen desert composition, a crème fraiche composition, a curd composition, a cottage cheese composition, or a cream cheese composition.

12. A colloidal suspension comprising: recombinant beta-casein protein, and at least one lipid; wherein the suspension does not contain an organoleptically functional amount of beta-lactoglobulin.

13. The colloidal suspension of embodiment 12, wherein the suspension is a non-Newtonian fluid.

14. The colloidal suspension of embodiment 12 or 13, which is characterized as a shear thinning fluid with an apparent viscosity greater than 10 centipoise, at a shear rate of 1 sec⁻¹.

15. The colloidal suspension of any one of embodiments 12-14, wherein the suspension is an aqueous suspension.

16. The colloidal suspension of any one of embodiments 12-15, wherein the suspension does not comprise any additional casein proteins.

17. The colloidal suspension of any one of embodiments 12-15, wherein the composition comprises at least one additional casein protein.

18. The colloidal suspension of embodiment 17, wherein at least 80% by weight of the total casein protein in the composition is beta-casein.

19. The colloidal suspension of embodiment 17 or 18, wherein the at least one additional casein protein is selected from kappa-casein, para-kappa-casein, beta-casein, alpha-S1-casein and alpha-S2-casein.

20. The colloidal suspension of embodiment 17 or 18, wherein the at least one additional casein protein is kappa-casein or para-kappa casein.

21. The colloidal suspension of any one of embodiments 12-20, wherein the recombinant beta-casein is plant-expressed.

22. The colloidal suspension of any one of embodiments 12-21, wherein the composition comprises a fusion protein comprising the recombinant beta-casein.

### Embodiment Set Number 24: Cheese Compositions

1. A cheese composition comprising para-kappa-casein produced without the use of any enzyme that cleaves kappa-casein to para-kappa casein.

2. A substantially transparent plant-based cheese composition.

3. A cheese composition comprising a recombinant beta-casein protein; wherein the cheese composition has at least one of the following characteristics: a firmness of at least 150 grams, as determined by compressing a cylindrical-shaped sample of the cheese composition having a height of 3 cm and a diameter of 3 cm to a height of 1.5 cm at 5° C.; a melting point of about 35° C. to about 100° C.; or ability to stretch to at least 3 cm in length without breaking, as determined by heating a 100 gram mass of the cheese composition at a temperature of about 225° C. for 4 minutes and cooling to about 90° C. and pulling with a fork placed beneath the mass.

4. The cheese composition of embodiment 141, wherein the composition does not comprise any additional casein proteins.

5. The cheese composition of embodiment 141, wherein the composition comprises at least one additional casein protein.

6. The cheese composition of embodiment 143, wherein at least 80% by weight of the total casein protein in the composition is beta-casein.

7. The cheese composition of embodiment 143, wherein at least 90% by weight of the total casein protein in the composition is beta-casein.

8. The cheese composition of embodiment 143, wherein at least 95% by weight of the total casein protein in the composition is beta-casein.

9. The cheese composition of embodiment 143, wherein the at least one additional casein protein is selected from kappa-casein, para-kappa-casein, beta-casein, alpha-S1-casein and alpha-S2-casein.

10. The cheese composition of embodiment 143, wherein the at least one additional casein protein is kappa-casein.

11. The cheese composition of embodiment 143, wherein the at least one additional casein protein is para-kappa casein.

12. The cheese composition of any one of embodiments 141-149, wherein the recombinant beta-casein is plant-expressed.

13. The cheese composition of embodiment 150, wherein the recombinant beta-casein is expressed in a soybean.

14. The cheese composition of any one of embodiments 143-149, wherein all caseins in the composition are plant-expressed.

15. The cheese composition of any one of embodiments 141-152, wherein the recombinant casein protein is derived from a fusion protein.

16. The cheese composition of any one of embodiments 141-153, wherein the composition does not contain an organoleptically functional amount of beta-lactoglobulin.

17. The cheese composition of any one of embodiments 141-154, wherein the composition has the ability to stretch to at least 3 cm in length without breaking, as determined by heating a 100 gram mass of the composition at a temperature of 225° C. for 4 minutes and cooling to about 90° C. and pulling with a fork placed beneath the mass.

18. The cheese composition of any one of embodiments 141-155, wherein the composition has the ability to stretch to at least 3 cm in length without breaking, as determined by heating a 100 gram mass of the composition at a temperature of 225° C. for 4 minutes and cooling to about 90° C. and pulling with a fork placed beneath the mass; and a firmness of at least 150 grams, as determined by compressing a cylindrical-shaped sample of the cheese composition having a height of 3 cm and a diameter of 3 cm to a height of 1.5 cm at 5° C.

19. The cheese composition of any one of embodiments 141-156, wherein the composition comprises at least one lipid and at least one salt.

20. The cheese composition of any one of embodiments 141-157, wherein the composition comprises calcium.

21. The cheese composition of embodiment 158, wherein the composition comprises calcium at a concentration of about 0.01% to about 2% by weight.

22. The cheese composition of any one of embodiments 141-159, wherein the composition has a pH of about 5.2 to about 5.9.

23. The cheese composition of any one of embodiments 141-160, wherein the composition comprises at least one organoleptic properties similar to cheese selected from the group consisting of taste, appearance, mouthfeel, structure, texture, density, elasticity, springiness, coagulation, binding, leavening, aeration, foaming, creaminess, and emulsification.

24. A method of making the cheese composition of embodiments 141-161, the method comprising expressing the recombinant beta-casein protein in a plant, extracting the beta-casein from the plant, and combining the beta-casein with at least one lipid and/or salt.

25. A cheese composition comprising a recombinant beta-casein protein; wherein the cheese composition has ability to stretch to at least 3 cm in length without breaking, as determined by heating a 100 gram mass of the composition at a temperature of 225° C. for 4 minutes and cooling to about 90° C. and pulling with a fork placed beneath the mass.

26. The cheese composition of embodiment 163, wherein the composition does not comprise any additional casein proteins.

27. The cheese composition of embodiment 163, wherein the composition comprises at least one additional casein protein, and wherein at least 80% by weight of the total casein protein in the composition is beta-casein.

28. The cheese composition of embodiment 165, wherein the at least one additional casein protein is kappa-casein or para-kappa casein.

29. The cheese composition of any one of embodiments 163-166, wherein the recombinant beta-casein is plant-expressed.

30. The cheese composition of any one of embodiments 165-167, wherein the recombinant casein protein is derived from a fusion protein.

31. The cheese composition of any one of embodiments 163-168, wherein the composition has at least one of the following characteristics: a firmness of at least 150 grams, as determined by compressing a cylindrical-shaped sample of the cheese composition having a height of 3 cm and a diameter of 3 cm to a height of 1.5 cm at 5° C.; or a melting point of about 35° C. to about 100° C.

32. A method of making the cheese composition of any one of embodiments 163-169, the method comprising expressing the recombinant beta-casein protein in a plant, extracting the beta-casein from the plant, and combining the beta-casein with at least one lipid and/or salt.

### Embodiment Set Number 25: Fusion Proteins

1. A recombinant fusion protein comprising: (i) a first milk protein; and (ii) a second milk protein.

1.1 The recombinant fusion protein of embodiment 1, wherein the first milk protein and/or the second milk protein lacks an animal secretion signal peptide.

1.2 The recombinant fusion protein of embodiment 1, wherein the first milk protein and/or the second milk protein is a truncated milk protein, lacking an animal secretion signal peptide.

2. The recombinant fusion protein of any one embodiment 1-1.2, wherein at least one of the first milk protein and the second milk protein is α-S1 casein, α-S2 casein, β-casein, κ-casein, para-κ-casein, β-lactoglobulin, α-lactalbumin, lysozyme, lactoferrin, lactoperoxidase, serum albumin, or an immunoglobulin.

3. The recombinant fusion protein of any one of embodiments 1-1.2, wherein at least one of the first milk protein and the second milk protein is β-lactoglobulin.

4. The recombinant fusion protein of any one of embodiments 1-1.2, wherein at least one of the first milk protein and the second milk protein is α-S1 casein, (t-S2 casein, (3-casein, κ-casein, or para-κ-casein.

5. The recombinant fusion protein of any one of embodiments 1-1.2, wherein: i) the first milk protein is α-S1 casein, α-S2 casein, β-casein, κ-casein, or para-κ-casein; and ii) the second milk protein is α-S1 casein, α-S2 casein, β-casein, κ-casein, or para-κ-casein.

6. The recombinant fusion protein of any one of embodiments 1-1.2, wherein at least one of the first milk protein and the second milk protein is κ-casein and comprises the sequence of SEQ ID NO: 4, or a sequence at least 90% identical thereto.

7. The recombinant fusion protein of any one of embodiments 1-1.2, wherein at least one of the first milk protein and the second milk protein is para-κ-casein and comprises the sequence of SEQ ID NO: 2, or a sequence at least 90% identical thereto.

8. The recombinant fusion protein of any one of embodiments 1-1.2, wherein at least one of the first milk protein and the second milk protein is β-casein and comprises the sequence of SEQ ID NO: 6, or a sequence at least 90% identical thereto.

9. The recombinant fusion protein of any one of embodiments 1-1.2, wherein at least one of the first milk protein and the second milk protein is α-S1 casein and comprises the sequence SEQ ID NO: 8, or a sequence at least 90% identical thereto.

10. The recombinant fusion protein of any one of embodiments 1-1.2, wherein at least one of the first milk protein and the second milk protein is α-S2 casein and comprises the sequence SEQ ID NO: 84, or a sequence at least 90% identical thereto.

11. The recombinant fusion protein of any one of embodiments 1-1.2, wherein the first milk protein and the second milk protein are different proteins.

12. The recombinant fusion protein of any one of embodiments 1-1.2, wherein the first milk protein and the second milk protein are the same proteins.

13. The recombinant fusion protein of any one of embodiments 1-12, which is plant-expressed.

14. The recombinant fusion protein of embodiment 13, which is expressed in a soybean plant.

15. The recombinant fusion protein of any one of embodiments 1-14, which comprises a protease cleavage site.

16. The recombinant fusion protein of embodiment 15, wherein the protease cleavage site is a chymosin cleavage site.

17. A nucleic acid encoding the recombinant fusion protein of any one of embodiments 1-16.

18. The nucleic acid of embodiment 17, which is codon-optimized for expression in a plant.

19. The nucleic acid of embodiment 18, which is codon-optimized for expression in a soybean.

20. An expression vector comprising the nucleic acid molecule of any one of embodiments 17-19.

21. A host cell comprising the nucleic acid of any one of claims 17-19 or the expression vector of embodiment 20.

22. The host cell of embodiment 21, wherein the cell is a plant cell, bacterial cell, fungal cell, or mammalian cell.

23. The host cell of embodiment 22, wherein the plant cell is a soybean cell.

24. A plant stably transformed with the nucleic acid of any one of embodiments 17-19 or the expression vector of embodiment 20.

25. The plant of embodiment 24, wherein the fusion protein is expressed in an amount of 1% or higher per total protein weight of soluble protein extractable from the plant.

26. A method for making a fusion protein, the method comprising:
(a) transforming a host cell with the nucleic acid of any one of embodiments 17-19 or the expression vector of claim 20; and
(b) growing the transformed host cell under conditions wherein the fusion protein is expressed.

27. The method of embodiment 26, which comprises co-expressing in the host cell a protein capable of forming a protein body.

28. The method of embodiment 27, wherein the protein capable of forming a protein body is a prolamin selected from a gliadin, a hordein, a secalin, a zein, a kafirin, or an avenin.

29. The method of embodiment 26, which comprises expressing a kinase in the host cell.

30. The method of embodiment 26, wherein expression of one or more proteases is knocked down or knocked out in the cell.

31. The method of any one of embodiments 26-30, wherein the fusion protein is expressed in an amount of 1% or higher per total protein weight of soluble protein extractable from the plant.

31.1 The method of any one of embodiments 26-30, wherein the fusion protein is expressed in an amount of 2%, 3%, 4%, 5%, 6%, 7%, 8%, 9%, 10%, 11%, 12%, 13%, 14%, 15%, 16%, 17%, 18%, 19%, 20% or higher per total protein weight of soluble protein extractable from the plant.

31.2 The method of any one of embodiments 26-31.1, wherein the fusion protein is expressed at least 1, 2, 3, 4, 5, 6, 7, 8, 9, 10, 11, 12, 13, 14, 15, 16, 17, 18, 19, 20, 21, 22, 23, 24, 25, 50, 75, 100, 125, 150, 175, 200 or more fold higher than a control plant expressing only the first or second milk protein individually.

32. A transgenic plant comprising the recombinant fusion protein of any one of embodiments 1-16, the nucleic acid of any one of embodiments 17-20, or the expression vector of embodiment 20.

33. The transgenic plant of embodiment 32, which is a soybean plant.

34. A method for stably expressing the recombinant fusion protein of any one of embodiments 1-16 in a plant, the method comprising: (i) transforming a plant with a plant transformation vector comprising an expression cassette comprising a nucleic acid molecule encoding the fusion protein; and (ii) growing the transformed plant under conditions wherein the recombinant fusion protein is expressed.

35. The method of embodiment 34, wherein the fusion protein is expressed in an amount of 1% or higher per total protein weight of soluble protein extractable from the plant.

36. A seed processing composition, comprising the fusion protein of any one of embodiments 1-16.

37. A food composition comprising the fusion protein of any one of embodiments 1-16.

38. The food composition of embodiment 37, which is selected from the group consisting of cheese and processed cheese products, yogurt and fermented dairy products, directly acidified counterparts of fermented dairy products, cottage cheese dressing, frozen dairy products, frozen desserts, desserts, baked goods, toppings, icings, fillings, low-fat spreads, dairy-based dry mixes, soups, sauces, salad dressing, geriatric nutrition, creams and creamers, analog dairy products, follow-up formula, baby formula, infant formula, milk, dairy beverages, acid dairy drinks, smoothies, milk tea, butter, margarine, butter alternatives, growing up milks, low-lactose products and beverages, medical and clinical nutrition products, protein/nutrition bar applications, sports beverages, confections, meat products, analog meat products, meal replacement beverages, weight management food and beverages, cultured buttermilk, sour cream, yogurt, skyr, leben, lassi, kefir, powder containing a milk protein, and low-lactose products.

39. The food composition of embodiment 37, which is a cheese composition.

40. The food composition of embodiment 39, wherein the cheese composition has the ability to stretch to at least 3 cm in length without breaking, as determined by heating a 100 gram mass of the composition at a temperature of 225° C. for 4 minutes and cooling to about 90° C. and pulling with a fork placed beneath the mass.

41. The food composition of embodiment 39 or 40, comprising a total amount of casein protein; wherein about 32% to 100% by weight of the total amount of casein protein in the food composition is beta-casein.

42. A method of making a food composition, comprising combining the fusion protein of any one of embodiments 1-16 into a food composition.

43. An alternative dairy food composition comprising: i) the recombinant fusion protein of any one of embodiments 1-16; and ii) at least one lipid, wherein the recombinant fusion protein confers on the alternative dairy food composition one or more characteristics of a dairy food product selected from the group consisting of: taste, aroma, appearance, handling, mouthfeel, density, structure, texture, elasticity, springiness, coagulation, binding, leavening, aeration, foaming, creaminess and emulsification.

44. The alternative dairy food composition of embodiment 43, wherein the alternative dairy food composition does not comprise any other milk proteins.

45. The alternative dairy food composition of embodiment 43 or 44, which comprises calcium at a concentration of about 0.01 to about 2% by weight.

46. The alternative diary food composition of any one of embodiments 43-45, comprising a total amount of casein protein; wherein about 32% to 100% by weight of the total amount of casein protein in the food composition is beta-casein.

47. The alternative diary food composition of any one of embodiments 43-46, wherein the composition has a pH of about 5.2 to about 5.9.

48. The alternative diary food composition of any one of embodiments 43-47, which is selected from the group consisting of cheese and processed cheese products, yogurt and fermented dairy products, directly acidified counterparts of fermented dairy products, cottage cheese dressing, frozen dairy products, frozen desserts, desserts, baked goods, toppings, icings, fillings, low-fat spreads, dairy-based dry mixes, soups, sauces, salad dressing, geriatric nutrition, creams and creamers, analog dairy products, follow-up formula, baby formula, infant formula, milk, dairy beverages, acid dairy drinks, smoothies, milk tea, butter, margarine, butter alternatives, growing up milks, low-lactose products and beverages, medical and clinical nutrition products, protein/nutrition bar applications, sports beverages, confections, meat products, analog meat products, meal replacement beverages, weight management food and beverages, cultured buttermilk, sour cream, yogurt, skyr, leben, lassi, kefir, powder containing a milk protein, and low-lactose products.

49. The alternative diary food composition of any one of embodiments 43-47, which is a cheese composition.

50. A solid phase, protein-stabilized emulsion comprising the fusion protein of any one of embodiments 1-16, wherein the emulsion has the ability to stretch to at least 3 cm in length without breaking, as determined by heating a 100 gram mass of the emulsion to a temperature of about 225° C. for 4 minutes and cooling to about 90° C. and pulling with a fork placed beneath the mass.

51. A colloidal suspension comprising the fusion protein of any one of embodiments 1-16; wherein the colloidal suspension has at least one, at least two, or at least three characteristics that are substantially similar to bovine milk selected from taste, appearance, mouthfeel, structure, texture, density, elasticity, springiness, coagulation, binding, leavening, aeration, foaming, creaminess, and emulsification.

### Embodiment Set Number 26: Agronomic Yield and Testa Color

1. A method for increasing yield of recombinant milk protein production per acre in soybean, comprising: providing to a locus a plurality of transgenic soybean seed, wherein the transgenic soybean seed comprise a recombinant DNA construct encoding a fusion protein comprising at least one milk protein, and wherein the plurality of transgenic soybean seed produce in the aggregate at least 2, 4, 6, 6.3, 8, 10, 12, 14, 16, 18, 20, 22, 24, 26, 28, 30, 32, 34, 36, 38, 40, 42, 44, 46, 48, or 50, pounds of recombinant milk protein per acre.

2. The method of embodiment 1, wherein the plurality of transgenic soybean seed produce in the aggregate at least 60, 70, 80, 90, 100, 150, 200, 250, 300, 350, 400, 450, 500, 550, 600, 650, 700, 750, 800, or 840 pounds of recombinant milk protein per acre

3. A method for increasing yield of recombinant milk protein production per bushel in soybean, comprising: providing to a locus a plurality of transgenic soybean seed, wherein the transgenic soybean seed comprise a recombinant DNA construct encoding a fusion protein comprising at least one milk protein, and wherein the plurality of transgenic soybean seed produce in the aggregate at least 0.21 pounds of recombinant milk protein per bushel.

4. The method of embodiment 3, wherein the plurality of transgenic soybean seed produce in the aggregate at least 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, or 10.5 pounds of recombinant milk protein per bushel.

5. A method for increasing the amount of recombinant milk protein produced per soybean seed, comprising: providing to a locus a transgenic soybean seed, wherein the transgenic soybean seed comprises a recombinant DNA construct encoding a fusion protein comprising at least one milk protein, and wherein the transgenic soybean seed produces at least 0.5 mg of recombinant milk protein per soybean seed.

6. The method of embodiment 5, wherein the transgenic soybean seed produces at least 0.7, 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, 25.0, 26.0, or 26.5 mg of recombinant milk protein per soybean seed.

7. A method for increasing yield of recombinant milk protein production per acre in soybean, comprising:
a. providing to a locus a plurality of transgenic soybean seed at a density of at least about 50,000 seeds per acre; and
b. growing the plurality of transgenic soybean seed until plant maturity and fruit pod formation,
wherein each transgenic soybean seed in the mature fruit pod produces at least 0.5 mg of recombinant milk protein per soybean seed.

7.1 The method of any one of embodiments 1-7, wherein the at least one milk protein in the fusion protein lacks an animal secretion signal peptide.

7.2 The method of any one of embodiments 1-7, wherein the at least one milk protein in the fusion protein is a truncated milk protein, lacking an animal secretion signal peptide.

8. A method for increasing yield of recombinant milk protein production per acre in soybean, comprising: providing to a locus a plurality of genetically modified soybean seed, wherein the plurality of genetically modified soybean seed produce in the aggregate at least 2 or 6.3 pounds of recombinant milk protein per acre.

9. The method of embodiment 8, wherein the plurality of genetically modified soybean seed produce in the aggregate at least 6.3, 10, 20, 30, 40, 50, 60, 70, 80, 90, 100, 150, 200, 250, 300, 350, 400, 450, 500, 550, 600, 650, 700, 750, 800, or 840 pounds of recombinant milk protein per acre

10. A method for increasing yield of recombinant milk protein production per bushel in soybean, comprising: providing to a locus a plurality of genetically modified soybean seed, wherein the plurality of genetically modified soybean seed produce in the aggregate at least 0.21 pounds of recombinant milk protein per bushel.

11. The method of embodiment 10, wherein the plurality of genetically modified soybean seed produce in the aggregate at least 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, or 10.5 pounds of recombinant milk protein per bushel.

12. A method for increasing the amount of recombinant milk protein produced per soybean seed, comprising: providing to a locus a genetically modified soybean seed, wherein the genetically modified soybean seed produces at least 0.5 mg of recombinant milk protein per soybean seed.

13. The method of embodiment 12, wherein the genetically modified soybean seed produces at least 0.7, 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, 25.0, 26.0, or 26.5 mg of recombinant milk protein per soybean seed.

14. A method for increasing yield of recombinant milk protein production per acre in soybean, comprising:
a. providing to a locus a plurality of genetically modified soybean seed at a density of at least about 50,000 seeds per acre; and
b. growing the plurality of genetically modified soybean seed until plant maturity and fruit pod formation,
   wherein each genetically modified soybean seed in the mature fruit pod produces at least 0.5 mg of recombinant milk protein per soybean seed.

15. The method of any one of embodiments 8 to 14, wherein the genetically modified soybean seed comprises at least one of the following genetic modifications:
a. a recombinant DNA construct encoding a fusion protein comprising at least one milk protein;
b. a recombinant DNA construct encoding a protein capable of forming a protein body;
c. a recombinant DNA construct encoding a prolamin;
d. a first recombinant DNA construct encoding a milk protein and a second recombinant DNA construct encoding a prolamin;
e. a recombinant DNA construct encoding a milk protein that has been modified to have an amino acid sequence different from the native animal expressed milk protein;
f. a recombinant DNA construct encoding a milk protein that has been modified to promote addition of a post-translational modification;
g. a recombinant DNA construct encoding a milk protein that has been modified to prevent addition of a post-translational modification;
h. a recombinant DNA construct encoding an enzyme that alters post-translational modification of protein;
i. a recombinant DNA construct encoding an enzyme capable of modifying a protein;
j. a recombinant DNA construct encoding a kinase; or
k. a genetic modification that modulates the expression of a plant protease.

16. A method for increasing yield of recombinant milk protein production per acre in a plant, comprising: providing to a locus a plurality of transgenic plant seed, wherein the transgenic plant seed comprise a recombinant DNA construct encoding a fusion protein comprising at least one milk protein, and wherein the plurality of transgenic plant seed produce in the aggregate at least 2 or 6.3 pounds of recombinant milk protein per acre.

17. The method of embodiment 16, wherein the plurality of transgenic plant seed produce in the aggregate at least 6.3, 10, 20, 30, 40, 50, 60, 70, 80, 90, 100, 150, 200, 250, 300, 350, 400, 450, 500, 550, 600, 650, 700, 750, 800, or 840 pounds of recombinant milk protein per acre

18. A method for increasing yield of recombinant milk protein production per acre in a plant, comprising: providing to a locus a plurality of genetically modified plant seed, wherein the plurality of genetically modified plant seed produce in the aggregate at least 2 pounds of recombinant milk protein per acre.

19. The method of embodiment 18, wherein the plurality of genetically modified plant seed produce in the aggregate at least 6.3, 10, 20, 30, 40, 50, 60, 70, 80, 90, 100, 150, 200, 250, 300, 350, 400, 450, 500, 550, 600, 650, 700, 750, 800, or 840 pounds of recombinant milk protein per acre

20. The method of any one of embodiments 16 to 19, wherein the plant seed comprises at least one of the following genetic modifications:
a. a recombinant DNA construct encoding a fusion protein comprising at least one milk protein;
b. a recombinant DNA construct encoding a protein capable of forming a protein body;
c. a recombinant DNA construct encoding a prolamin;
d. a first recombinant DNA construct encoding a milk protein and a second recombinant DNA construct encoding a prolamin;
e. a recombinant DNA construct encoding a milk protein that has been modified to have an amino acid sequence different from the native animal expressed milk protein;
f. a recombinant DNA construct encoding a milk protein that has been modified to promote addition of a post-translational modification;
g. a recombinant DNA construct encoding a milk protein that has been modified to prevent addition of a post-translational modification;
h. a recombinant DNA construct encoding an enzyme that alters post-translational modification of protein;
i. a recombinant DNA construct encoding an enzyme capable of modifying a protein;
j. a recombinant DNA construct encoding a kinase; or
k. a genetic modification that modulates the expression of a plant protease.

21. The method of any one of embodiments 16 to 19, wherein the plant is a soybean.

22. A plant seed comprising:
a) a recombinant mammalian milk protein selected from the group consisting of: α-S1 casein, α-S2 casein, β-casein, κ-casein, para-κ-casein, and β-lactoglobulin.

22.1 The plant seed/method of any one of embodiments 1-22, wherein the plant seed comprises a testa with an exogenously-induced color.

22.1.1 The plant seed of any one of embodiments 22-22.1, wherein the recombinant milk protein lacks an animal secretion signal peptide.

22.1.2 The plant seed of any one of embodiments 22-22.1, wherein the recombinant milk protein is a truncated milk protein, lacking an animal secretion signal peptide.

22.2 The plant seed of embodiment 22.1, wherein the exogenously-induced color is not present in the seed endosperm.

22.3 The plant seed of any one of embodiments 22.1-22.2, wherein the exogenously-induced color is only present in the testa.

22.3.1 The plant seed of any one of embodiments 22.1-22.3, wherein the exogenously-induced color is encoded in a genetic modification.

22.32 The plant seed of any one of embodiments 22.1 to 22.3, wherein the exogenously-induced color occurs due to a genetic modification in a metabolic pathway selected from the group consisting of flavonoids, carotenoids, and alkaloids.

22.4 The plant seed of any one of embodiments 22.1 to 22.3, wherein the exogenously-induced color occurs due to a genetic modification in the anthocyanin metabolic pathway.

22.5 The plant seed of any one of embodiments 22.1 to 22.4, wherein the genetic modification comprises overexpression of an R2R3 or bHLH transcription factor.

22.5.1 The plant seed of embodiment 22.5, wherein the R2R3 or bHLH transcription factor is selected from the group consisting of: AtMybA, MoroMybA, VvMybA, PamMybA.1, PamMybA.5, and GmMYBA2, and GmTT8a.

22.5.2 The plant seed of embodiment 22.5, wherein the R2R3 transcription factor is selected from the group consisting of: AT1G56650, KT992776, JX470201, KT992773, KT992775, Glyma02g16670, and Glyma09g36983.

22.6 The plant seed of any one of embodiments 22.1-22.5.2, wherein the genetic modification comprises over-expression of a Chalcone Synthase (CHS) protein.

22.7 The plant seed of embodiment 22.6, wherein a re-coded native CHS protein is expressed.

22.7.1 The plant seed of embodiment 22.7, wherein the re-coded native CHS protein is encoded by a sequence selected from the group consisting of: SEQ ID NO: 887, SEQ ID NO: 888, and SEQ ID NO: 889.

22.8 The plant seed of embodiment 22.6 wherein an exogenous CHS protein is expressed.

22.8.1 The plant seed of embodiment 22.8, wherein the exogenous CHS protein is encoded by a sequence selected from the group consisting of AT5G13930, AT5G13930, AT5G13930, AB030004.

22.8.2 The plant seed of embodiment 22.8, wherein the exogenous CHS protein is encoded by a sequence selected from the group consisting of: SEQ ID NO: 890, SEQ ID NO: 891, SEQ ID NO: 892, SEQ ID NO: 893, SEQ ID NO: 894, and SEQ ID NO: 895.

22.8.3 The plant seed of embodiment 22.8, wherein the exogenous CHS protein is recoded.

22.9 The plant seed of embodiment 22.6 or 22.7, wherein the promoter operably linked to the gene encoding for the CHS protein comprises is expressed with a testa specific promoter.

22.10 The plant seed of any one of embodiments 22.5-22.7, wherein the R2R3 or bHLH transcription factor is overexpressed with a testa specific promoter.

22.11 The plant seed of embodiment 22.9 or 22.10, wherein the testa promoter is selected from the promoters of any one of Glyma11g03010, Glyma01g42350, Glyma03g40310, Glyma01g26230, Glyma14g07940, Glyma06g00680, Glyma07g28940, Glyma08g19580, Glyma06g02500, Glyma14g34480, Glyma09g30910, or Glyma06g42780.

22.11.1 The plant seed of any one of embodiments 22.3.1-22.11 wherein the genetic modification comprises expression of an R2R3 transcription factor and a bHLH transcription factor.

22.11.2 The plant seed of any one of embodiments 22.3.1-22.11 wherein the genetic modification comprises expression of an R2R3 transcription factor and a recoded native or exogenous CHS.

22.11.3 The plant seed of any one of embodiments 22.3.1-22.11 wherein the genetic modification comprises expression of an R2R3 transcription factor and a bHLH transcription factor and expression of a recoded native or exogenous CHS.

22.12. The plant seed of any one of embodiments 22-22.11.3, wherein the recombinant mammalian milk protein is expressed in a fusion with a fusion partner.

22.13. The plant seed of any one of embodiments 22.12, wherein the fusion protein is a milk protein.

22.14. The plant seed of embodiment 22.13, wherein the fusion partner is selected from the group consisting of: α-S1 casein, α-S2 casein, β-casein, κ-casein, para-κ-casein, and β-lactoglobulin.

22.15 The plant seed of any one of embodiments 21-22.14, wherein the mammalian milk protein is encoded by an exogenous nucleic acid.

22.15.1 The plant seed of embodiment 22.15, wherein the exogenous nucleic acid and the genetic modification are within the same integrated T-DNA.

22.15.2 The plant seed of embodiment 22.15, wherein the exogenous nucleic acid and the genetic modification are on different integrated T-DNAs.

22.16 The plant seed of embodiment 22.15, wherein the exogenous nucleic acid and the genetic modification are located within 10, 9, 8, 7, 6, 5, 4, 3, 2, 1, 0.5, or 0.1 centimorgans of each other in the genome of the plant.

22.17 The plant seed of embodiment 22.15, wherein the exogenous nucleic acid and the genetic modification are located within 3 kb, 2 kb, 1 kb, 500 bp, 250 bp or 100 bp of each other in the genome of the plant.

22.18 The plant seed of any one of embodiments 21-22.17, wherein the plant seed comprises a balancing genetic modification, that reduces the plant seed's use of one or more amino acids present in the recombinant mammalian milk protein.

22.19 The plant seed of embodiment 22.18, wherein the balancing genetic modification is a downregulation of a protein selected from the group consisting of 0-conglycinin α, β-conglycinin α′, β-conglycinin R, Cysteine Protease, Glycinin 1, Glycinin 2, Glycinin 3, Glycinin 4, Glycinin 5, Glycinin 7, Kunitz-type Trypsin inhibitor, and lectin.

22.19.1 The plant seed of embodiment 22.18, wherein the balancing genetic modification comprises a genomic modification of a promoter sequence of a gene selected from the group consisting of β-conglycinin α, β-conglycinin α′, β-conglycinin R, 4 Beta-Conglycinin, Cysteine Protease, Glycinin 1, Glycinin 2, Glycinin 3, Glycinin 4, Glycinin 5, Glycinin 6, Glycinin 7, Kunitz Trypsin inhibitor 3, 3 Lipoxygenase, and lectin.

22.19.2 The plant seed of embodiment 22.19.1, wherein the promoter is selected from the group consisting of: Glycinin1, Glycinin 4, Beta-Conglycinin α′ subunit, and Kunitz Tripsin Inhibitor 3.

22.20 The plant seed of embodiment 22.18 or 22.19, wherein the balancing genetic modification, comprises a partial deletion, sequence insertion, or sequence replacement in the gene encoding for a protein selected from the group consisting of β-conglycinin α, 0-conglycinin α′, β-conglycinin R, Cysteine Protease, Glycinin 1, Glycinin 2, Glycinin 3, Glycinin 4, Glycinin 5, Glycinin 7, Kunitz-type Trypsin inhibitor, and lectin.

22.20 The plant seed of embodiment 22.18 or 22.19, wherein the balancing genetic modification, comprises a partial deletion, sequence insertion, or sequence replacement in the promoter ofthe gene encoding for a protein selected from the group consisting of β-conglycinin α, β-conglycinin α′, β-conglycinin R, Cysteine Protease, Glycinin 1, Glycinin 2, Glycinin 3, Glycinin 4, Glycinin 5, Glycinin 7, Kunitz-type Trypsin inhibitor, and lectin.

22.21 The plant seed of any one of embodiments 21-22.20 wherein at least one of the genetic modifications is made using a gene-editing technology.

22.22 The plant seed of embodiment 22.21, wherein the gene-editing technology is a CRISPR/Cas system.

22.23 The plant seed of embodiment 22.22, wherein the CRISPR system comprises a nucleic acid molecule and an enzymatic protein, wherein the nucleic acid molecule is a guide RNA (gRNA) molecule and the enzymatic protein is a Cas protein or Cas ortholog.

22.24 A guide nucleic acid comprising a spacer sequence that is complementary to a sequence of a promoter of a gene selected from the group consisting of Glycinin1, Glycinin 4, Beta-Conglycinin α′ subunit, and Kunitz Tripsin Inhibitor 3.

22.25 A guide nucleic acid comprising a spacer sequence that is complementary to a sequence of a gene selected from the group consisting of: β-conglycinin α, β-conglycinin α′, β-conglycinin R, 4 Beta-Conglycinin, Cysteine Protease, Glycinin 1, Glycinin 2, Glycinin 3, Glycinin 4, Glycinin 5, Glycinin 6, Glycinin 7, Kunitz Trypsin inhibitor 3, 3 Lipoxygenase, and lectin.

### Embodiment Set Number 27: Testa Color

1. A plant seed, wherein the plant seed comprises a testa with an exogenously-induced color.

2. The plant seed of embodiment 1, wherein the exogenously-induced color is not present in the seed endosperm.

3. The plant seed of any one of embodiments 1-2, wherein the exogenously-induced color is only present in the testa.

4. The plant seed of any one of embodiments 1-3, wherein the exogenously-induced color is encoded in a genetic modification.

5. The plant seed of any one of embodiments 1-3, wherein the exogenously-induced color occurs due to a genetic modification in a metabolic pathway selected from the group consisting of flavonoids, carotenoids, and alkaloids.

6. The plant seed of any one of embodiments 1-3, wherein the exogenously-induced color occurs due to a genetic modification in the anthocyanin metabolic pathway.

7. The plant seed of any one of embodiments 1-6, wherein the genetic modification comprises overexpression of an R2R3 or bHLH transcription factor.

8. The plant seed of embodiment 7, wherein the R2R3 or bHLH transcription factor is selected from the group consisting of: AtMybA, MoroMybA, VvMybA, PamMybA.1, PamMybA.5, and GmMYBA2, and GmTT8a.

9. The plant seed of embodiment 7, wherein the R2R3 transcription factor is selected from the group consisting of: AT1G56650, KT992776, JX470201, KT992773, KT992775, Glyma02g16670, and Glyma09g36983.

10. The plant seed of any one of embodiments 1-9, wherein the genetic modification comprises over-expression of a Chalcone Synthase (CHS) protein.

11. The plant seed of embodiment 10, wherein a re-coded native CHS protein is expressed.

12. The plant seed of embodiment 11, wherein the re-coded native CHS protein is encoded by a sequence selected from the group consisting of: SEQ ID NO: 887, SEQ ID NO: 888, and SEQ ID NO: 889.

13. The plant seed of embodiment 10, wherein an exogenous CHS protein is expressed.

14. The plant seed of embodiment 13, wherein the exogenous CHS protein is encoded by a sequence selected from the group consisting of AT5G13930, AT5G13930, AT5G13930, AB030004.

15. The plant seed of embodiment 13, wherein the exogenous CHS protein is encoded by a sequence selected from the group consisting of: SEQ ID NO: 890, SEQ ID NO: 891, SEQ ID NO: 892, SEQ ID NO: 893, SEQ ID NO: 894, and SEQ ID NO: 895.

16. The plant seed of embodiment 13, wherein the exogenous CHS protein is recoded.

17. The plant seed of any one of embodiments 10-16, wherein the promoter operably linked to the gene encoding for the CHS protein comprises comprises a testa specific promoter.

18. The plant seed of any one of embodiments 7-17, wherein the R2R3 or bHLH transcription factor is overexpressed with a testa specific promoter.

19. The plant seed of embodiment 17 or 18, wherein the testa promoter is selected from the promoters of any one of Glymal1g03010, Glyma01g42350, Glyma03g40310, Glyma01g26230, Glyma14g07940, Glyma06g00680, Glyma07g28940, Glyma08g19580, Glyma06g02500, Glyma14g34480, Glyma09g30910, or Glyma06g42780.

20. The plant seed of any one of embodiments 4-19, wherein the genetic modification comprises expression of an R2R3 transcription factor and a bHLH transcription factor.

21. The plant seed of any one of embodiments 4-19, wherein the genetic modification comprises expression of an R2R3 transcription factor and a recoded native or exogenous CHS.

22. The plant seed of any one of embodiments 4-19, wherein the genetic modification comprises expression of an R2R3 transcription factor and a bHLH transcription factor and expression of a recoded native or exogenous CHS.

### Embodiment Set Number 28: Compositions of Interest (Ratio)

23. A composition comprising:
a) a recombinant mammalian milk protein;
b) a second protein selected from the group consisting of 7S globulin glycinin, 11S globulin, Lipoxygenase, and Kunitz Trypsin Inhibitor;

wherein the milk protein and second protein are present at a w/w ratio of at least 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, or 1:1.23.1 The composition of embodiment 23, wherein the recombinant mammalian milk protein lacks an animal secretion signal peptide.

23.2 The composition of embodiment 23, wherein the recombinant mammalian milk protein is a truncated milk protein, lacking an animal secretion signal peptide.

24. The composition of any one of embodiments 23-23.2, wherein the milk protein is selected from the group consisting of α-S1 casein, α-S2 casein, β-casein, κ-casein, para-κ-casein, and β-lactoglobulin.

24.1 The composition of any one of embodiments 23-23.2, wherein the milk protein is selected from the group consisting of: α-S1 casein, α-S2 casein, β-casein, κ-casein, para-κ-casein, β-lactoglobulin, α-lactalbumin, lysozyme, lactoferrin, lactoperoxidase, serum albumin, and an immunoglobulin

25. The composition of any one of embodiments 23-24.1, wherein the composition does not have lactose.

25.1 The composition of any one of embodiments 23-25, wherein said composition does not have one or more proteins present in cow milk.

25.2 The composition of embodiment 25.1, wherein the one or more proteins present in cow milk are selected from the group consisting of α-S1 casein, α-S2 casein, (3-casein, κ-casein, para-κ-casein, β-lactoglobulin, α-lactalbumin, lysozyme, lactoferrin, lactoperoxidase, serum albumin, and an immunoglobulin.

25.3 The composition of embodiment 25.1, wherein the one or more proteins present in cow milk are selected from the group consisting of α-lactalbumin, lysozyme, lactoferrin, lactoperoxidase, serum albumin, and an immunoglobulin.

26. The composition of any one of embodiments 23-25.3, further comprising: c) chlorophyll.

27. The composition of any one of embodiments 23-26, wherein the recombinant milk protein is expressed in a fusion protein with a fusion partner

28. The composition of embodiment 27, wherein the fusion partner is a milk protein.

29. The composition of embodiment 28, wherein the fusion partner is selected from the group consisting of: α-S1 casein, α-S2 casein, β-casein, κ-casein, para-κ-casein, and β-lactoglobulin.

29.1 The composition of embodiment 28, wherein the fusion partner is selected from the group consisting of: α-S1 casein, α-S2 casein, β-casein, κ-casein, para-κ-casein, β-lactoglobulin, α-lactalbumin, lysozyme, lactoferrin, lactoperoxidase, serum albumin, and an immunoglobulin

30. The composition of embodiment 27, wherein the fusion protein is selected from the group consisting of:
a) Beta-casein-AlphaS1-casein-AlphaS1-casein-Beta-casein;
b) Beta-casein-Beta-casein-Kappa-casein-Beta-lactoglobulin;
c) Beta-casein-Beta-casein-Beta-casein-Beta-casein; and
d) Gamma-Zein-Beta-casein.

31. The composition of any one of any one of embodiments 23-29.1, wherein the composition is a coffee creamer, a cheese, a substitute milk product.

31. The composition of any one of embodiments 23-29.1, further comprising: a nucleic acid encoding glycinin.

32. The composition of any one of embodiment 23, further comprising: a nucleic acid encoding lipoxygenase.

33. The composition of embodiment 23, further comprising: a nucleic acid encoding kunitz trypsin inhibitor.

33.5 The composition of embodiment 23, wherein the recombinant mammalian milk protein comprises reduced phosphorylation as compared to a corresponding mammalian milk protein in a bovine as determined by mass spectrometry.

34. The composition of any one of embodiments 23-33, wherein the composition does not have a fatty acid profile comprising:

| | |
|---|---|
| palmitic acid (16:0) | about 10% |
| stearic acid (18:0) | about 4% |
| oleic acid (18:1) | about 18% |
| linoleic acid (18:2) | about 55% |
| linolenic acid (18:3) | about 13% |

wherein the fatty acid profile represents (wt/wt) percent over total fatty acids.

35. The composition of any one of embodiments 23-33, wherein the composition has a fatty acid profile comprising: palmitic acid (16:0) about 23%-32%

| | |
|---|---|
| palmitic acid (16:0) | about 23%-32% |

wherein the fatty acid profile represents (wt/wt) percent over total fatty acids.

35.1 The composition of any one of embodiments 23-33, wherein the composition has a fatty acid profile comprising: palmitic acid (16:0) about 13%-42%

| | |
|---|---|
| palmitic acid (16:0) | about 13%-42% |

wherein the fatty acid profile represents (wt/wt) percent over total fatty acids.

36. The composition of any one of embodiments 23-35, wherein the composition has a fatty acid profile comprising:

| | |
|---|---|
| stearic acid (18:0) | about 21%-26% |

wherein the fatty acid profile represents (wt/wt) percent over total fatty acids.

36.1 The composition of any one of embodiments 23-35, wherein the composition has a fatty acid profile comprising:

| | |
|---|---|
| stearic acid (18:0) | about 11%-36% |

wherein the fatty acid profile represents (wt/wt) percent over total fatty acids.

37. The composition of any one of embodiments 23-36, wherein the composition has a fatty acid profile comprising:

| | |
|---|---|
| oleic acid (18:1) | about 17%-27% |

wherein the fatty acid profile represents (wt/wt) percent over total fatty acids.

37.1 The composition of any one of embodiments 23-36, wherein the composition has a fatty acid profile comprising:

| | |
|---|---|
| oleic acid (18:1) | about 7%-37% |

wherein the fatty acid profile represents (wt/wt) percent over total fatty acids.

38. The composition of any one of embodiments 23-37, wherein the composition has a fatty acid profile comprising:

| | |
|---|---|
| linoleic acid (18:2) | about 0.5%-3.1% |

wherein the fatty acid profile represents (wt/wt) percent over total fatty acids.

38.1 The composition of any one of embodiments 23-37, wherein the composition has a fatty acid profile comprising:

| | |
|---|---|
| linoleic acid (18:2) | about 0.01%-13.1% |

wherein the fatty acid profile represents (wt/wt) percent over total fatty acids.

39. The composition of any one of embodiments 23-33, wherein the composition has a fatty acid profile comprising any one or more of:

| | |
|---|---|
| a) palmitic acid (16:0) | about 23%-32% |
| b) stearic acid (18:0) | about 21%-26% |
| c) oleic acid (18:1) | about 17%-27% |
| d) linoleic acid (18:2) | about 0.5%-3.1% |

wherein the fatty acid profile represents (wt/wt) percent over total fatty acids.

40. The composition of any one of embodiments 23-33, wherein the composition has a fatty acid profile comprising:

| | |
|---|---|
| palmitic acid (16:0) | about 23%-32% |
| stearic acid (18:0) | about 21%-26% |
| oleic acid (18:1) | about 17%-27% |
| linoleic acid (18:2) | about 0.5%-3.1% |

wherein the fatty acid profile represents (wt/wt) percent over total fatty acids.

40.1 The composition of any one of embodiments 23-33, wherein the composition has a fatty acid profile comprising:

| | |
|---|---|
| palmitic acid (16:0) | about 13%-42% |
| stearic acid (18:0) | about 11%-36% |
| oleic acid (18:1) | about 7%-37% |
| linoleic acid (18:2) | about 0.01%-13.1% |

wherein the fatty acid profile represents (wt/wt) percent over total fatty acids.

41. The composition of any one of embodiments 23-33, wherein the composition has a fatty acid profile comprising:

| | |
|---|---|
| palmitic acid (16:0), | about 28% |
| stearic acid (18:0), | about 11% |
| oleic acid (18:1), | about 22% |
| linoleic acid (18:2) | about 2.5% |

wherein the fatty acid profile represents (wt/wt) percent over total fatty acids.

42. The composition of any one of embodiments 23-33, wherein the composition has a fatty acid profile comprising any one or more of:

| | |
|---|---|
| a) C4:0 fatty acids | about 1%-7% |
| b) C6:0 fatty acids | about 0.5-6% |
| c) C8:0 fatty acids | about 0.5%-4.5% |
| d) C10:0 fatty acids | about 0.4%-6% |
| e) C12:0 fatty acids | about 0.4%-6.5% |
| f) C14:0 fatty acids | about 7%-13% |
| g) C16:0 fatty acids | about 26%-32% |
| h) C16:1 fatty acids | about 0.2%-4.2% |
| i) C18:0 fatty acids | about 14%-21% |
| j) C18:1 fatty acids | about 18%-26% |
| k) C18:2 fatty acids | about 0.5%-7% |

wherein the fatty acid profile represents (wt/wt) percent over total fatty acids.

42.1 The composition of any one of embodiments 23-33, wherein the composition has a fatty acid profile comprising any one or more of:

| | |
|---|---|
| a) C4:0 fatty acids | about .01%-17% |
| b) C6:0 fatty acids | about 0.01-16% |
| c) C8:0 fatty acids | about 0.01%-14.5% |
| d) C10:0 fatty acids | about 0.01%-16% |
| e) C12:0 fatty acids | about 0.01%-16.5% |
| f) C14:0 fatty acids | about 0.1%-23% |
| g) C16:0 fatty acids | about 16%-42% |
| h) C16:1 fatty acids | about 0.01%-14.2% |
| i) C18:0 fatty acids | about 4%-31% |
| j) C18:1 fatty acids | about 8%-36% |
| k) C18:2 fatty acids | about 0.01%-17% |

wherein the fatty acid profile represents (wt/wt) percent over total fatty acids.

43. The composition of any one of embodiments 23-33, wherein the composition has a fatty acid profile comprising:

| | |
|---|---|
| C4:0 fatty acids | about 1%-7% |
| C6:0 fatty acids | about 0.5-6% |
| C8:0 fatty acids | about 0.5%-4.5% |
| C10:0 fatty acids | about 0.4%-6% |
| C12:0 fatty acids | about 0.4%-6.5% |
| C14:0 fatty acids | about 7%-13% |
| C16:0 fatty acids | about 26%-32% |
| C16:1 fatty acids | about 0.2%-4.2% |
| C18:0 fatty acids | about 14%-21% |
| C18:1 fatty acids | about 18%-26% |
| C18:2 fatty acids | about 0.5%-7% |

wherein the fatty acid profile represents (wt/wt) percent over total fatty acids.

43.1 The composition of any one of embodiments 23-33, wherein the composition has a fatty acid profile comprising:

| | |
|---|---|
| C4:0 fatty acids | about .01%-17% |
| C6:0 fatty acids | about 0.01-16% |
| C8:0 fatty acids | about 0.01%-14.5% |
| C10:0 fatty acids | about 0.01%-16% |
| C12:0 fatty acids | about 0.01%-16.5% |
| C14:0 fatty acids | about 0.1%-23% |
| C16:0 fatty acids | about 16%-42% |
| C16:1 fatty acids | about 0.01%-14.2% |
| C18:0 fatty acids | about 4%-31% |
| C18:1 fatty acids | about 8%-36% |
| C18:2 fatty acids | about 0.01%-17% |

wherein the fatty acid profile represents (wt/wt) percent over total fatty acids.

44. The composition of any one of embodiments 23-33, wherein the composition has a fatty acid profile comprising:

| | |
|---|---|
| C4:0 fatty acids | about 4.34% |
| C6:0 fatty acids | about 2.70% |
| C8:0 fatty acids | about 1.60% |
| C10:0 fatty acids | about 3.40% |
| C12:0 fatty acids | about 3.48% |
| C14:0 fatty acids | about 10.00% |
| C16:0 fatty acids | about 29.20% |
| C16:1 fatty acids | about 1.29% |
| C18:0 fatty acids | about 17.47% |
| C18:1 fatty acids | about 22.84% |
| C18:2 fatty acids | about 3.67% |

wherein the fatty acid profile represents (wt/wt) percent over total fatty acids.

45. The composition of any one of embodiments 23-44, wherein the composition comprises added short chain fatty acids.

46. The composition of embodiment 45, wherein the added short chain fatty acids are added in the form of a plant oil.

47. The composition of embodiment 46, wherein the plant oil is selected from the group consisting of soybean oil, palm oil, coconut oil, almond oil, avocado oil, cocoa butter oil, corn oil, cottonseed oil, flax seed oil, grapeseed oil, hemp oil, olive oil, palm kernel oil, peanut oil, pumpkin seed oil, rice bran oil, safflower seed oil, sesame seed oil, sunflower seed oil, walnut oil, and combinations thereof.

96. A method of expressing a transgene in a plant cell, the method comprising: (i) providing a plant cell lacking at least one endogenous promoter; (ii) transforming the plant cell with a nucleic acid comprising a promoter and a transgene, wherein the promoter has the same sequence as the endogenous promoter; and (ii) maintaining the plant cell under conditions wherein the transgene is expressed.

97. The method of embodiment 96, wherein the plant cell is a dicot cell from soybean, lima bean, Arabidopsis, tobacco, rice, maize, barley, sorghum, wheat or oat.

98. The method of embodiment 96, wherein the plant cell is a monocot cell from turf grass, maize, rice, oat, wheat, barley, sorghum, orchid, iris, lily, onion, palm, or duckweed.

99. The method of embodiment 96, wherein the promoter is the Glycinin 1 or Seed 2 promoter.

100. The method of embodiment 96, wherein the transgene is a mammalian, avian, or plant gene.

101. The method of embodiment 100, wherein the transgene encodes a milk protein selected from α-S1 casein, α-S2 casein, β-casein, κ-casein, para-κ-casein, β-lactoglobulin, α-lactalbumin, lysozyme, lactoferrin, lactoperoxidase, serum albumin, or an immunoglobulin.

102. The method of embodiment 100, wherein transgene encodes an avian protein selected from ovalbumin, ovotransferrin, ovoglobulin, and lysozyme.

103. The method of embodiment 100, wherein the transgene encodes a plant protein selected from oleosins, leghemoglobin, extension-like protein family, prolamin, glutenin, gamma-kafirin preprotein, α-globulin, basic 7S globulin precursor, 2S albumin, (3-conglycinins, glycinins, canein, zein, patatin, kunitz-trypsin inhibitor, bowman-birk inhibitor, and cystatine.

104. A plant cell lacking at least one endogenous promoter; wherein the plant cell comprises a nucleic acid comprising a promoter and a transgene; wherein the promoter has the same sequence as the endogenous promoter.

### Embodiment Set Number 29: Compositions of Interest (Percent by Weight or Soluble Protein)

1. A composition comprising:
a) a recombinant mammalian milk protein;
b) soy protein;
wherein the milk protein comprises at least 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, or 35% by weight of the composition.

1.1 A composition comprising:
a) a recombinant mammalian milk protein;
b) soy protein; wherein the milk protein comprises at least 0.2%, 0.3%, 0.4%, 0.5%, 0.6%, 0.7%, 0.80%, 0.9%, 10%, 2%, 3%, 4%, 5%, 6%, 7%, 8%, 9%, 10%, 110%, 12%, 130%, 14%, 15%, 16%, 17%, 18%, 19%, 20%, 21%, 22%, 23%, 24%, 25%, 26%, 27%, 28%, 29%, 30%, 31%, 32%, 33%, 34%, or 35% by dry weight of the composition.

1.2 A composition comprising:
a) a recombinant mammalian milk protein;
b) soy protein;
wherein the milk protein comprises at least 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, or 35% of the total protein content of the composition.

2. The composition of any one of embodiment 1-1.3, comprising c) a fat or lipid.

3. The composition of any one of embodiments 1-2, wherein the milk protein comprises at least 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, or 35% of the total soluble protein content of the composition.

4. The composition of any one of embodiments 1-3, wherein the recombinant mammalian milk protein lacks an animal secretion signal peptide.

5. The composition of any one of embodiments 1-3, wherein the recombinant mammalian milk protein is a truncated milk protein, lacking an animal secretion signal peptide.

6. The composition of any one of embodiments 1-5, wherein the recombinant mammalian milk protein was expressed from a dicotyledenous plant.

7. The composition of any one of embodiments 1-5, wherein the recombinant mammalian milk protein was expressed in a soybean.

8. The composition of any one of embodiments 1-5, wherein the recombinant mammalian milk protein and the soybean protein were expressed together in soybean.

9. The composition of any one of embodiments 1-8, wherein the milk protein is selected from the group consisting of: α-S1 casein, α-S2 casein, β-casein, κ-casein, para-κ-casein, and β-lactoglobulin.

10. The composition of any one of embodiments 1-8, wherein the milk protein is selected from the group consisting of: α-S1 casein, α-S2 casein, β-casein, κ-casein, para-κ-casein, β-lactoglobulin, α-lactalbumin, lysozyme, lactoferrin, lactoperoxidase, serum albumin, and an immunoglobulin

11. The composition of any one of embodiments 1-10, wherein the composition does not have lactose.

12. The composition of any one of embodiments 1-11, wherein said composition does not have one or more proteins present in cow milk.

13. The composition of any one of embodiments 1-11, wherein said composition does not have the full complement of casein proteins present in cow milk.

14. The composition of embodiment 12, wherein the one or more proteins present in cow milk are selected from the group consisting of α-S1 casein, α-S2 casein, β-casein, κ-casein, para-κ-casein, β-lactoglobulin, α-lactalbumin, lysozyme, lactoferrin, lactoperoxidase, serum albumin, and an immunoglobulin.

15. The composition of embodiment 13, wherein the full complement of caseins present in cow milk consists essentially of α-S1 casein, α-S2 casein, β-casein, and κ-casein.

16. The composition of embodiment 12, wherein the one or more proteins present in cow milk are selected from the group consisting of α-lactalbumin, lysozyme, lactoferrin, lactoperoxidase, serum albumin, and an immunoglobulin.

17. The composition of any one of embodiments 1-16, further comprising: c) chlorophyll.

18. The composition of any one of embodiments 1-17, wherein the recombinant milk protein is expressed in a fusion protein with a fusion partner

19. The composition of embodiment 18, wherein the fusion partner is a milk protein.

20. The composition of embodiment 19, wherein the fusion partner is selected from the group consisting of: α-S1 casein, α-S2 casein, β-casein, κ-casein, para-κ-casein, and 0-lactoglobulin.

21. The composition of embodiment 19, wherein the fusion partner is selected from the group consisting of: α-S1 casein, α-S2 casein, β-casein, κ-casein, para-κ-casein, 0-lactoglobulin, α-lactalbumin, lysozyme, lactoferrin, lactoperoxidase, serum albumin, and an immunoglobulin

22. The composition of embodiment 18, wherein the fusion protein is selected from the group consisting of
a) Beta-casein-AlphaS1-casein-AlphaS1-casein-Beta-casein;
b) Beta-casein-Beta-casein-Kappa-casein-Beta-lactoglobulin;
c) Beta-casein-Beta-casein-Beta-casein-Beta-casein; and
d) Gamma-Zein-Beta-casein.

23. The composition of any one of embodiments 1-22, wherein the composition is a coffee creamer, a cheese, or a substitute milk product.

23. The composition of any one of embodiments 1-22, wherein the composition is capable of producing a food product with more dairy-like properties than a control food product lacking the recombinant milk protein.

24. The composition of any one of embodiments 1-23, wherein the composition is capable of producing a food product with greater stretch and melt properties than a control food product lacking the recombinant milk protein.

25. The composition of any one of embodiments 1-24, further comprising: a nucleic acid encoding glycinin.

26. The composition of any one of embodiments 1-25, further comprising: a nucleic acid encoding lipoxygenase.

27. The composition of any one of embodiments 1-26, further comprising: a nucleic acid encoding kunitz trypsin inhibitor.

28 The composition of any one of embodiments 1-27, wherein the recombinant mammalian milk protein comprises reduced phosphorylation as compared to a corresponding mammalian milk protein in a bovine as determined by mass spectrometry.

### Embodiment Set Number 30 (Universal Vector)

1. A vector for expressing a gene of interest, said vector comprising in order from 5′ to 3′: a) a seed-specific promoter; b) a signal peptide; and c) a double terminator.

2. The vector of embodiment 1, wherein the seed-specific promoter is selected from the group consisting of PvPhas, BnNap, AtOle1, GmSeed2, GmSeed3, GmSeed5, GmSeed6, GmSeed7, GmSeed8, GmSeed10, GmSeed11, GmSeed12, pBCON, GmCEP1-L, GmTHIC, GmBg7S1, GmGRD, GmOLEA, GmOLER, Gm2S-1, and GmBBld-II.

3. The vector of embodiment 1, wherein the seed-specific promoter is GmSeed2.

4. The vector of embodiment 1, wherein the seed-specific promoter is GmSeed2 and comprises the sequence of SEQ ID NO: 19, or a sequence at least 90% identical thereto.

4.1 The vector of any one of embodiments 1-4, wherein the signal peptide is a plant signal peptide.

4.2 The vector of embodiment 4.1, wherein the signal peptide directs translationally fused proteins to the secretory pathway.

5. The vector of any one of embodiments 1-4.2, wherein the signal peptide is selected from the group consisting of GmSCB1, StPat21, 2Sss, Sig2, Sig12, Sig8, Sig10, Sig11, and Coixss.

6. The vector of any one of embodiments 1-5, wherein the signal peptide is Sig2.

7. The vector of any one of embodiments 1-6, wherein the signal peptide is Sig2 and comprises the sequence of SEQ ID NO: 814, or a sequence at least 90% identical thereto.

8. The vector of any one of embodiments 1-7, wherein the vector comprises a terminator isolated or derived from a gene encoding Nopaline synthase, Arc5-1, an Extensin, Rb7 matrix attachment region, a Heat shock protein, Ubiquitin 10, Ubiquitin 3, and M6 matrix attachment region.

9. The vector of any one of embodiments 1-8, wherein the vector comprises an Rb7 matrix attachment region.

10. The vector of any one of embodiments 1-9, wherein the vector comprises an M6 matrix attachment region.

11. The vector of any one of embodiments 1-10, wherein the dual terminator is AtHSP:AtUbi10.

12. The vector of any one of embodiments 1-11, wherein the dual terminator is AtHSP:AtUbi10 and comprises the sequence of SEQ ID NO: 141, or a sequence at least 90% at least 95%, at least 96%, at least 97%, at least 98%, or at least 99% identical thereto.

13. The vector of any one of embodiments 1-12, wherein the dual terminator is EU:TM6.

14. The vector of any one of embodiments 1-13, wherein the dual terminator is EU:TM6 and comprises the sequence of SEQ ID NO: 146, or a sequence at least 90% at least 95%, at least 96%, at least 97%, at least 98%, or at least 99% identical thereto.

15. The vector of any one of embodiments 1-14, wherein the vector encodes for an endoplasmic retention signal.

16. The vector of embodiment 15, wherein the endoplasmic retention signal is a KDEL sequence.

17. The vector of embodiment 16, wherein the KDEL sequence comprises SEQ ID NO: 23.

18. The vector of any one of embodiments 1-17, wherein the gene of interest encodes for a mammalian milk protein.

19. The vector of any one of embodiments 1-17, wherein the gene of interest encodes a fusion protein comprising a first and a second mammalian milk protein.

20. The vector of any one of embodiments 18-19, wherein the mammalian milk protein is selected from the group consisting of α-S1 casein, α-S2 casein, β-casein, κ-casein, para-κ-casein, β-lactoglobulin, α-lactalbumin, lysozyme, lactoferrin, lactoperoxidase, serum albumin, and an immunoglobulin.

22. A vector for expressing a gene of interest, said vector comprising in order from 5′ to 3′: a) a GmSeed2 seed-specific promoter that comprises the sequence of SEQ ID NO: 19, or a sequence at least 90% identical thereto; b) a sequence encoding for a Sig2 signal peptide that comprises the sequence of SEQ ID NO: 814, or a sequence at least 90% identical thereto; and c) a AtHSP:AtUbi10 double terminator that comprises the sequence of SEQ ID NO: 141, or a sequence at least 90% at least 95%, at least 96%, at least 97%, at least 98%, or at least 99% identical thereto.

23. The vector of embodiment 22, wherein the endoplasmic retention signal is a KDEL sequence.

24. The vector of embodiment 23, wherein the KDEL sequence comprises SEQ ID NO: 23.

25. The vector of any one of embodiments 22-24, wherein the vector comprises an Rb7 matrix attachment region.

26. The vector of claim 22, wherein the vector comprises an M6 matrix attachment region.

27. A method of expressing a transgene in a plant cell, the method comprising: (i) providing a plant cell lacking at least one endogenous promoter; (ii) transforming the plant cell with a nucleic acid comprising a promoter and a transgene, wherein the promoter has the same sequence as the endogenous promoter; and (ii) maintaining the plant cell under conditions wherein the transgene is expressed.

97. The method of embodiment 96, wherein the plant cell is a dicot cell from soybean, lima bean, Arabidopsis, tobacco, rice, maize, barley, sorghum, wheat or oat.

98. The method of embodiment 96, wherein the plant cell is a monocot cell from turf grass, maize, rice, oat, wheat, barley, sorghum, orchid, iris, lily, onion, palm, or duckweed.

99. The method of embodiment 96, wherein the promoter is the Glycinin 1 or Seed 2 promoter.

100. The method of embodiment 96, wherein the transgene is a mammalian, avian, or plant gene.

101. The method of embodiment 100, wherein the transgene encodes a milk protein selected from α-S1 casein, α-S2 casein, β-casein, κ-casein, para-κ-casein, β-lactoglobulin, α-lactalbumin, lysozyme, lactoferrin, lactoperoxidase, serum albumin, or an immunoglobulin.

28. The method of embodiment 27, wherein transgene encodes an avian protein selected from ovalbumin, ovotransferrin, ovoglobulin, and lysozyme.

29. The method of embodiment 27, wherein the transgene encodes a plant protein selected from oleosins, leghemoglobin, extension-like protein family, prolamin, glutenin, gamma-kafirin preprotein, α-globulin, basic 7S globulin precursor, 2S albumin, 0-conglycinins, glycinins, canein, zein, patatin, kunitz-trypsin inhibitor, bowman-birk inhibitor, and cystatine.

30. A plant cell lacking at least one endogenous promoter; wherein the plant cell comprises a nucleic acid comprising a promoter and a transgene; wherein the promoter has the same sequence as the endogenous promoter.

### Embodiment Set 32 (Amino Acid Rebalancing)

1. A transgenic plant, plant part, plant cell, or plant tissue culture comprising a recombinant construct, said construct comprising (i) a tissue-specific promoter, (ii) a first nucleic acid sequence encoding a bovine milk protein, which is operably linked to said promoter, and (iii) a termination sequence; wherein tissue-specific promoter is selected from the group consisting of an nucleic acid having at least 95% sequence identity to SEQ ID NOs: 19-30; wherein said milk protein is selected from the group consisting of α-S1 casein, α-S2 casein, β-casein, κ-casein, α-lactalbumin, β-lactoglobulin, and lysozyme; and wherein said plant, plant part, plant cell, or plant tissue culture expresses said milk protein, and has enhanced milk protein production compared to untransformed control plants.

2. The transgenic plant, plant part, plant cell, or plant tissue culture of embodiment 1, wherein said construct further comprises an ER retention signal sequence.

3. The transgenic plant, plant part, plant cell, or plant tissue culture of embodiment 1, wherein said construct further comprises (i) at least one second nucleic acid sequence encoding a guide RNA and (ii) a third nucleic acid sequence encoding a Clustered regularly interspaced short palindromic repeats (CRISPR) endonuclease, wherein said guide RNA is capable of forming a complex with said CRISPR endonuclease, and wherein said complex is capable of binding to and creating a double strand break in a genomic target sequence of said plant genome.

4. The transgenic plant, plant part, plant cell, or plant tissue culture of embodiment 3, wherein the target sequence is a nucleic acid sequence encoding a seed storage protein.

5. The transgenic plant, plant part, plant cell, or plant tissue culture of embodiment 1, wherein the plant is selected from the group consisting of soybean, lima bean, common bean, green pea, chickpea, Pisum sativum, rapeseed, Arabidopsis thaliana, tobacco, duckweed, hybrid aspen, muskmelon, potato, tomato, canola, crambe, mustard, castor bean, sesame, linseed, maize, barley, peanut, alfalfa, wheat, rice, oat, sorghum, rye, tritordeum, millet, fescue, perennial ryegrass, sugarcane, cranberry, papaya, banana, safflower, oil palms, flax, muskmelon, apple, cucumber, dendrobium, gladiolus, chrysanthemum, liliacea, cotton, eucalyptus, sunflower, turfgrass, sugarbeet, coffee, and dioscorea.

6. The transgenic plant, plant part, plant cell, or plant tissue culture of embodiment 1, wherein the plant is soybean, lima bean, common bean, green pea, chickpea, Pisum sativum, or rapeseed.

7. The transgenic plant, plant part, plant cell, or plant tissue culture of embodiment 1, wherein the first nucleic acid is expressed in an tissue specific manner.

8. The transformed plant, plant part, plant cell, or plant tissue culture of embodiment 7, wherein the nucleic acid is expressed in a seed.

9. The transgenic plant, plant part, plant cell, or plant tissue culture of embodiment 1, wherein the first nucleic acid sequence encoding κ-casein is codon-optimized.

10. The transgenic plant, plant part, plant cell, or plant tissue culture of embodiment 1, wherein the first nucleic acid sequence encoding β-casein is codon-optimized.

11. The transgenic plant, plant part, plant cell, or plant tissue culture of embodiment 1, wherein the first nucleic acid sequence encoding α-S1 casein is codon-optimized.

12. The transgenic plant, plant part, plant cell, or plant tissue culture of embodiment 1, wherein the first nucleic acid sequence encoding α-S2 casein is codon-optimized.

13. The transgenic plant, plant part, plant cell, or plant tissue culture of embodiment 1, wherein the first nucleic acid sequence encoding α-lactalbumin is codon-optimized.

14. The transgenic plant, plant part, plant cell, or plant tissue culture of embodiment 1, wherein the first nucleic acid sequence encoding β-lactoglobulin is codon-optimized.

15. The transgenic plant, plant part, plant cell, or plant tissue culture of embodiment 1, wherein the first nucleic acid sequence encoding lysozyme is codon-optimized.

16. The transgenic plant, plant part, plant cell, or plant tissue culture of embodiment 1, wherein the first nucleic acid sequence encodes κ-casein having at least 95% sequence identity to SEQ ID No:10 or SEQ ID No:11.

17. The transgenic plant, plant part, plant cell, or plant tissue culture of embodiment 1, wherein the first nucleic acid sequence encoding β-casein having at least 95% sequence identity to SEQ ID No:12 or SEQ ID No:13.

18. The transgenic plant, plant part, plant cell, or plant tissue culture of embodiment 1, wherein the first nucleic acid sequence encoding α-S1 having at least 95% sequence identity to SEQ ID No:14.

19. The transgenic plant, plant part, plant cell, or plant tissue culture of embodiment 1, wherein the first nucleic acid sequence encoding α-S2 casein having at least 95% sequence identity to SEQ ID No:15.

20. The transgenic plant, plant part, plant cell, or plant tissue culture of embodiment 1, wherein the first nucleic acid sequence encoding α-lactalbumin having at least 95% sequence identity to SEQ ID No:16.

21. The transgenic plant, plant part, plant cell, or plant tissue culture of embodiment 1, wherein the first nucleic acid sequence encoding β-lactoglobulin having at least 95% sequence identity to SEQ ID No: 17.

22. The transgenic plant, plant part, plant cell, or plant tissue culture of embodiment 1, wherein the first nucleic acid sequence encoding lysozyme having at least 95% sequence identity to SEQ ID No:18.

23. The transgenic plant, plant part, plant cell, or plant tissue culture of embodiment 1, wherein the termination sequence is a nopaline synthase (NOS) terminator.

24. The transgenic plant, plant part, plant cell, or plant tissue culture of embodiment 3, wherein the transgenic plant produces a seed with a rebalanced storage protein level when a gene encoding the storage protein is mutated by a gene-editing technique.

25. The transgenic plant, plant part, plant cell, or plant tissue culture of embodiment 3, wherein the bovine milk protein level increases in the seed with the rebalanced storage protein level, when compared to a seed produced from a non-rebalanced transgenic plant; wherein the non-rebalanced transgenic plant has the recombinant DNA construct without mutation in the gene encoding the storage protein.

26. A method of producing said transgenic plant of embodiment 1, said method comprising the steps of: (a) introducing at least one expression cassette capable of expressing a milk protein into a plant, a part thereof, or a cell thereof; (b) obtaining the transgenic plant, the part thereof, or the cell thereof, which stably expresses the milk protein; (c) cultivating the transgenic plant, the part thereof, or the cell thereof; and (d) harvesting the transgenic plant, the part thereof, or the cell thereof.

27. A seed produced by the method of embodiment 26.

28. A method of producing a hybrid transgenic plant seed, wherein the method comprises crossing the plant of embodiment 1 with a different plant and harvesting the resultant hybrid plant seed.

29. A hybrid transgenic plant seed produced by method of embodiment 28.

30. A method of producing a bovine milk protein from a seed of said transgenic plant of embodiment 1, said method comprising the steps of: (a) extracting the bovine milk protein from the seed; and (b) purifying the bovine milk protein from the seed, wherein the bovine milk protein comprises α-S1 casein, α-S2 casein, β-casein, κ-casein, α-lactalbumin, β-lactoglobulin, and lysozyme; and wherein the bovine milk protein further comprises a proteolytic product of α-S1 casein, α-S2 casein, β-casein, κ-casein, α-lactalbumin, 0-lactoglobulin, and lysozyme.

31. A method of breeding transgenic plants to produce plants with enhanced milk protein production comprising: (a) making a cross between a first transgenic plant of embodiment 1 with a second plant to produce a F1 plant; (b) backcrossing the F1 plant to the second plant; and (c) repeating the backcrossing step one or more times to generate a near isogenic or isogenic line, wherein the expression cassette of embodiment 26 is integrated into the genome of the second plant and the near isogenic or isogenic line derived from the second plant with the nucleic acid sequences encoding the milk protein has enhanced milk protein production compared to untransformed control plants.

32. A food comprising a transgenic plant containing the nucleic acid of embodiment 1 or 3.

33. A food prepared from a transgenic plant containing the nucleic acid of embodiment 1 or 3.

34. A product prepared from a transgenic plant containing the nucleic acid of embodiment 1 or 3.

### Embodiment Set 33

1. A method for increasing yield of recombinant milk protein production per acre in soybean, comprising: providing to a locus a plurality of transgenic soybean seed, wherein the transgenic soybean seed comprise a recombinant DNA construct encoding a fusion protein comprising at least one milk protein, and wherein the plurality of transgenic soybean seed produce in the aggregate at least 2 pounds of recombinant milk protein per acre.

2. The method of embodiment 1, wherein the plurality of transgenic soybean seed produce in the aggregate at least 5, 10, 20, 30, 40, 50, 60, 70, 80, 90, 100, 150, 200, 250, 300, 350, or 400 pounds of recombinant milk protein per acre

3. A method for increasing yield of recombinant milk protein production per bushel in soybean, comprising: providing to a locus a plurality of transgenic soybean seed, wherein the transgenic soybean seed comprise a recombinant DNA construct encoding a fusion protein comprising at least one milk protein, and wherein the plurality of transgenic soybean seed produce in the aggregate at least 0.2 pounds of recombinant milk protein per bushel.

4. The method of embodiment 3, wherein the plurality of transgenic soybean seed produce in the aggregate at least 0.5, 1.0, 1.5, 2.0, 2.5, 3.0, 3.5, or 4.0 pounds of recombinant milk protein per bushel.

5. A method for increasing the amount of recombinant milk protein produced per soybean seed, comprising: providing to a locus a transgenic soybean seed, wherein the transgenic soybean seed comprises a recombinant DNA construct encoding a fusion protein comprising at least one milk protein, and wherein the transgenic soybean seed produces at least 0.35 mg of recombinant milk protein per soybean seed.

6. The method of embodiment 5, wherein the transgenic soybean seed produces at least 0.5, 1.0, 2.0, 3.0, 4.0, 5.0, 6.0, 7.0, or 8.0 mg of recombinant milk protein per soybean seed.

7. A method for increasing yield of recombinant milk protein production per acre in soybean, comprising: providing to a locus a plurality of transgenic soybean seed at a density of at least about 50,000 seeds per acre; and growing the plurality of transgenic soybean seed until plant maturity and fruit pod formation, wherein each transgenic soybean seed in the mature fruit pod produces at least 0.35 mg of recombinant milk protein per soybean seed.

8. A method for increasing yield of recombinant milk protein production per acre in soybean, comprising: providing to a locus a plurality of genetically modified soybean seed, wherein the plurality of genetically modified soybean seed produce in the aggregate at least 2 pounds of recombinant milk protein per acre.

9. The method of embodiment 8, wherein the plurality of genetically modified soybean seed produce in the aggregate at least 5, 10, 20, 30, 40, 50, 60, 70, 80, 90, 100, 150, 200, 250, 300, 350, or 400 pounds of recombinant milk protein per acre

10. A method for increasing yield of recombinant milk protein production per bushel in soybean, comprising: providing to a locus a plurality of genetically modified soybean seed, wherein the plurality of genetically modified soybean seed produce in the aggregate at least 0.2 pounds of recombinant milk protein per bushel.

11. The method of embodiment 10, wherein the plurality of genetically modified soybean seed produce in the aggregate at least 0.5, 1.0, 1.5, 2.0, 2.5, 3.0, 3.5, or 4.0 pounds of recombinant milk protein per bushel.

12. A method for increasing the amount of recombinant milk protein produced per soybean seed, comprising: providing to a locus a genetically modified soybean seed, wherein the genetically modified soybean seed produces at least 0.35 mg of recombinant milk protein per soybean seed.

13. The method of embodiment 12, wherein the genetically modified soybean seed produces at least 0.5, 1.0, 2.0, 3.0, 4.0, 5.0, 6.0, 7.0, or 8.0 mg of recombinant milk protein per soybean seed.

14. A method for increasing yield of recombinant milk protein production per acre in soybean, comprising: providing to a locus a plurality of genetically modified soybean seed at a density of at least about 50,000 seeds per acre; and growing the plurality of genetically modified soybean seed until plant maturity and fruit pod formation, wherein each genetically modified soybean seed in the mature fruit pod produces at least 0.35 mg of recombinant milk protein per soybean seed.

15. The method of any one of embodiments 8 to 14, wherein the genetically modified soybean seed comprises at least one of the following genetic modifications: a recombinant DNA construct encoding a fusion protein comprising at least one milk protein; a recombinant DNA construct encoding a protein capable of forming a protein body; a recombinant DNA construct encoding a prolamin; a first recombinant DNA construct encoding a milk protein and a second recombinant DNA construct encoding a prolamin; a recombinant DNA construct encoding a milk protein that has been modified to have an amino acid sequence different from the native animal expressed milk protein; a recombinant DNA construct encoding a milk protein that has been modified to promote addition of a post-translational modification; a recombinant DNA construct encoding a milk protein that has been modified to prevent addition of a post-translational modification; a recombinant DNA construct encoding an enzyme that alters post-translational modification of protein; a recombinant DNA construct encoding an enzyme capable of modifying a protein; a recombinant DNA construct encoding a kinase; or a genetic modification that modulates the expression of a plant protease.

16. A method for increasing yield of recombinant milk protein production per acre in a plant, comprising: providing to a locus a plurality of transgenic plant seed, wherein the transgenic plant seed comprise a recombinant DNA construct encoding a fusion protein comprising at least one milk protein, and wherein the plurality of transgenic plant seed produce in the aggregate at least 2 pounds of recombinant milk protein per acre.

17. The method of embodiment 16, wherein the plurality of transgenic plant seed produce in the aggregate at least 5, 10, 20, 30, 40, 50, 60, 70, 80, 90, 100, 150, 200, 250, 300, 350, or 400 pounds of recombinant milk protein per acre

18. A method for increasing yield of recombinant milk protein production per acre in a plant, comprising: providing to a locus a plurality of genetically modified plant seed, wherein the plurality of genetically modified plant seed produce in the aggregate at least 2 pounds of recombinant milk protein per acre.

19. The method of embodiment 18, wherein the plurality of genetically modified plant seed produce in the aggregate at least 5, 10, 20, 30, 40, 50, 60, 70, 80, 90, 100, 150, 200, 250, 300, 350, or 400 pounds of recombinant milk protein per acre

20. The method of any one of embodiments 16 to 19, wherein the plant seed comprises at least one of the following genetic modifications: a recombinant DNA construct encoding a fusion protein comprising at least one milk protein; a recombinant DNA construct encoding a protein capable of forming a protein body; a recombinant DNA construct encoding a prolamin; a first recombinant DNA construct encoding a milk protein and a second recombinant DNA construct encoding a prolamin; a recombinant DNA construct encoding a milk protein that has been modified to have an amino acid sequence different from the native animal expressed milk protein; a recombinant DNA construct encoding a milk protein that has been modified to promote addition of a post-translational modification; a recombinant DNA construct encoding a milk protein that has been modified to prevent addition of a post-translational modification; a recombinant DNA construct encoding an enzyme that alters post-translational modification of protein; a recombinant DNA construct encoding an enzyme capable of modifying a protein; a recombinant DNA construct encoding a kinase; or a genetic modification that modulates the expression of a plant protease.

21. The method of any one of embodiments 16 to 19, wherein the plant is a soybean.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world. Further, PCT International Publication No. WO 2018/187754 A1; U.S. Pat. Pub. No. US 2021/0222186 A1; U.S. Pat. Pub. No. US 2021/0010017 A1; U.S. Pat. Nos. 10,894,812; 11,304,743; 10,988,521; 10,947,552; and 11,072,797 are hereby incorporated by reference.

What is claimed is:

1. A food composition comprising:
a) a recombinant mammalian milk protein; and
b) a modified soy protein component comprising:
i) β-conglycinin (7S globulin); and
ii) 11S globulin (11s globulin);
wherein the modified soy protein component comprises a lower amount of 7S globulin than the amount present in a wild type soybean seed.

2. The food composition of claim 1, wherein the modified soy protein component comprises less than about 85% of the amount of 7S globulin than the amount present in soy protein from wild type soybean seed.

3. The food composition of claim 1, wherein the modified soy protein component comprises less than about 75% of the amount of 7S globulin than the amount present in soy protein from wild type soybean seed.

4. The food composition of claim 1, wherein the modified soy protein component comprises less than about 50% of the amount of 7S globulin than the amount present in soy protein from wild type soybean seed.

5. The food composition of claim 1, wherein the (w/w) ratio of recombinant mammalian milk protein to 11S globulin is at least 1:2.

6. The food composition of claim 1, wherein the (w/w) ratio of recombinant mammalian milk protein to 11S globulin is at least 1:1.

7. The food composition of claim 1, wherein the recombinant mammalian milk protein lacks an animal secretion signal peptide.

8. The food composition of claim 1, wherein the recombinant mammalian milk protein is selected from the group consisting of: α-S1 casein, α-S2 casein, β-casein, κ-casein, para-κ-casein, and β-lactoglobulin.

9. The food composition of claim 1, wherein the recombinant mammalian milk protein comprises a casein.

10. The food composition of claim 1, wherein the recombinant mammalian milk protein is selected from the group consisting of: α-S1 casein, α-S2 casein, β-casein, and κ-casein.

11. The food composition of claim 1, wherein the composition does not have lactose.

12. The food composition of claim 1, comprising:
c) chlorophyll.

13. The food composition of claim 1, wherein the recombinant mammalian milk protein is expressed in a fusion protein with a fusion partner.

14. The food composition of claim 13, wherein the fusion partner is a milk protein.

15. The food composition of claim 13, wherein the fusion partner is selected from the group consisting of: α-S1 casein, α-S2 casein, β-casein, κ-casein, para-κ-casein, zein, and 0-lactoglobulin.

16. The food composition of claim 13, wherein the fusion protein is selected from the group consisting of:
i) β-casein-α-S1-casein-α-S1-casein-β-casein;
ii) β-casein-β-casein-κ-casein-β-lactoglobulin;
iii) β-casein-β-casein-β-casein-β-casein; and
iv) Gamma-Zein-β-casein.

17. The food composition of claim 1, wherein the composition is a coffee creamer, a cheese, a substitute milk product.

18. The food composition of claim 1, further comprising: a nucleic acid encoding a glycinin.

19. The food composition of claim 1, wherein the recombinant mammalian milk protein exhibits reduced phosphorylation as compared to a corresponding mammalian milk protein in a bovine as determined by mass spectrometry.

20. A method for producing a plant-based dairy substitute, the method comprising the steps of:
a) providing soy protein comprising: and
i) a recombinant mammalian milk protein; and
ii) a soy protein component comprising:
1) β-conglycinin (7S globulin); and
2) 11S globulin (11s);
b) reducing 7S globulin content within the soy protein component.
